(19) United States
(12) Patent Application Publication
(10) Pub. No.: US 2010/0184032 A1
(43) Pub. Date: Jul. 22, 2010

(54) Stem-Progenitor Cell Specific Micro-Ribonucleic Acids and Uses Thereof

(75) Inventors: Robert Georgantas, Towson, MD (US); Curt I. Civin, Towson, MD (US); George Adrian Calin, Columbus, OH (US); Carlo Maria Croce, Columbus, OH (US)

Correspondence Address: Foley Hoag, LLP (w/JHV), World Trade Center West, 155 Seaport Blvd, Boston, MA, 02210-2600, US

(73) Assignee: THE JOHNS HOPKINS UNIVERSITY, BALTIMORE, MD (US)

(21) Appl. No.: 12/478,586

(22) Filed: Jun. 4, 2009

Related U.S. Application Data
(63) Continuation of application No. PCT/US2007/024845, filed on Dec. 4, 2007.
(60) Provisional application No. 60/872,764, filed on Dec. 4, 2006.

(51) Int. Cl. C12N 5/0789; C12Q 1/68

(52) U.S. Cl. 435/6; 435/377; 435/325

(57) ABSTRACT

Provided herein are methods and compositions for modulating the differentiation of incompletely differentiated cells, such as stem-progenitor cells, e.g., hematopoietic stem-progenitor cells. A method may comprise contacting a cell with an agononist or an antagonist of a miRNA comprising a nucleotide sequence from SEQ ID NO: 1-34.

### CROSS-REFERENCE TO RELATED APPLICATIONS

This application claims the benefit of U.S. Provisional Patent Application No. 60/872,764, filed on Dec. 4, 2006, the content of which is specifically incorporated by reference herein in its entirety.

### GOVERNMENTAL SUPPORT

This invention was made with governmental support under grant numbers CA70970 and CA60441, awarded by the U.S. National Institutes of Health. The government has certain rights in this invention.

### BACKGROUND

MicroRNAs (miRNAs) consist of a class of epigenetic elements that inhibit translation of mRNA to protein and also may result in gene silencing through chromatin remodeling (1). miRNAs have been identified in many organisms, including plants, Drosophila, rats, mice, and humans, where they have been shown to control major cellular processes, including metabolism, differentiation, and development (2). miRNAs have been implicated in the differentiation of mammalian blood cell lineages. miRNA-181 seems to bias mouse lymphoid progenitors toward B lymphoid development, and miRNA-146 and -223 bias toward T lymphopoiesis (3). miRNA-221 and -222 are critically involved in the negative control of human erythropoiesis (4) and miRNA-223 in the down-regulation of mouse granulopoiesis (5). Given that miRNAs regulate normal cellular functions, it is not surprising that miRNAs also are involved in carcinogenesis. miRNAs have been identified at deleted or amplified genomic regions or translocation breakpoints in human cancers (6); e.g., miRNA-15 and -16 are the critical genetic elements deleted from 13q14 chromosomal region in a subset of chronic lymphocytic leukemia cases (7).

### SUMMARY OF THE INVENTION

Provided herein are methods of modulating the differentiation or proliferation of an incompletely differentiated cell, e.g., a stem-progenitor cell, such as a hematopoietic cell. A method may comprise contacting a cell with a miRNA of one or more of SEQ ID NOS: 1 to 34 (Table 1). Contacting may comprise providing an miRNA to said cell, or providing an expression construct encoding said miRNA to said cell. The cell may be located in an animal subject, such as a human, or the cell may be contacted in vitro, wherein the method may comprise further culturing of said cell with or without reintroduction into an animal.

In yet another embodiment, there is provided a method of modulating the proliferation or differentiation of a cell, e.g., a hematopoietic cell, comprising contacting said cell with an agent that is an antagonist of the function or expression of the miRNAs of SEQ ID NOS: 1-34 (Tables 1 and 6). The cell may be contacted in vitro, wherein the method may comprise further culturing of said cell, with or without reintroduction into an animal. The agent may be a peptide, protein, DNA, RNA, antisense DNA, antisense RNA or small molecule.

In a further embodiment, there is provided a method of modulating differentiation of a cell, e.g., a hematopoietic cell, comprising inhibiting the function of one or more of the miRNAs of SEQ ID NOS: 1-34 (Tables 1 and 6). Inhibiting the function may comprise contacting the cell with one or more modified or unmodified antisense constructs directed to one or more of the miRNAs of SEQ ID NOS: 1-34. The cell may be located in an animal subject, such as a human, or the cell may be contacted in vitro, followed by culturing said cell in vitro, with or without reintroduction into an animal.

In still a further embodiment, there is provided a method of modulating differentiation of a cell, e.g., a hematopoietic cell, comprising providing to said cell an agonist of one or more of the miRNAs of SEQ ID NOS: 1-34 (Tables 1 and 6). The agonist may be one or more of the miRNAs of SEQ ID NOS: 1-34. The agonist may also be an expression cassette encoding one or more of the miRNAs of SEQ ID NOS: 1-34. The agonist may be a peptide, protein, nucleic acid, or small molecule that stimulates the expression of one or more of the miRNAs of SEQ ID NOS: 1-34. The cell may be located in an animal subject, such as a human, or the cell may be contacted in vitro, and may be followed by culturing said cell in vitro, with or without reintroduction into the animal.

Provided herein are method for modulating the differentiation of a cell, e.g., a stem-progenitor cell (SPC). A method may comprise contacting a stem-progenitor cell with one or more nucleic acids, wherein each nucleic acid comprises a nucleotide sequence that is at least about 90% identical to one or more of SEQ ID NOs: 1-34 or the complement thereof. Modulating the differentiation of a stem-progenitor cell may be inhibiting the differentiation of a stem-progenitor cell, in which case, the stem-progenitor cell is contacted with one or more nucleic acids, wherein each nucleic acid comprises a nucleotide sequence that is at least about 90% identical to one or more of SEQ ID NOs: 1-34. Modulating the differentiation of a stem-progenitor cell may be stimulating the differentiation of a stem-progenitor cell, in which case, the stem-progenitor cell is contacted with one or more nucleic acids, wherein each nucleic acid comprises a nucleotide sequence that is at least about 90% identical to one or more of the complements of SEQ ID NOs: 1-34. In one embodiment, the stem-progenitor cell is a hematopoietic stem-progenitor cell and the SEQ ID NOs are selected from the group consisting of the sequences of mir-128a, mir-181a, mir-146, mir-155, mir-24a, mir-17, mir-16, mir-103, mir-107, mir-223, mir-221 and mir-222 or the complement thereof The hematopoietic stem-progenitor cell may be a CD34+ cell.

In one embodiment, the differentiation of a hematopoietic stem-progenitor cell beyond the stage of multipotent progenitor cell (MPP) and/or before the stage of common lymphoid progenitor (CLP) or common myeloid progenitor (CMP) is inhibited, and the SEQ ID NOs are selected from the group consisting of mir-128a, mir-181a, mir-146, mir-155, mir-24a, and mir-17. In one embodiment, the differentiation of a hematopoietic stem-progenitor cell beyond the stage of MPP andor before the stage of CLP is inhibited, and the SEQ ID NO is that of mir-146. In one embodiment, the differentiation of a hematopoietic stem-progenitor cell beyond the stage of MPP and/or before the stage of CMP is inhibited, and the SEQ ID NOs are selected from the group consisting of mir-155, mir-24a or mir-17. In one embodiment, the differentiation of a hematopoietic stem-progenitor cell beyond the stage of CMP and/or before the stage of B/Mac bi-potential is inhibited, and the SEQ ID NOs are those of mir-103 or mir-107. In one embodiment, the differentiation of the hematopoietic stem-progenitor cell beyond the stage of CMP and/or before the stage of megakaryocyte-erythrocyte progenitor (MEP) is inhibited, and the SEQ ID NO is that of mir-16. In one embodiment, the differentiation of a hematopoietic stem-progenitor cell beyond the stage of granulocyte-macrophage progenitor cell (GMP) and/or before the stage of granulocytes is inhibited, and the SEQ ID NO is that of mir-223. In one embodiment, the differentiation of a hematopoietic stem-progenitor cell beyond the stage of MEP and/or before the stage of erythrocyte is inhibited, and the SEQ ID NO is that of mir-221 and/or mir-222.

In one embodiment, the differentiation of a hematopoietic stem-progenitor cell towards the stage of CLP and CMP is stimulated, and the SEQ ID NOs are selected from the group consisting of mir-128a, mir-181a, mir-146, mir-155, mir-24a, and mir-17. In one embodiment, the differentiation of a hematopoietic stem-progenitor cell towards the stage of CLP is stimulated, and the SEQ ID NO is that of mir-146. In one embodiment, the differentiation of a hematopoietic stem-progenitor cell towards the stage of CMP is stimulated, and the SEQ ID NOs are selected from the group consisting of mir-155, mir-24a or mir-17. In one embodiment, the differentiation of a hematopoietic stem-progenitor cell towards the stage of B/Mac bi-potential and GMP is stimulated, and the SEQ ID NOs are those of mir-103 or mir-107. In one embodiment, the differentiation of a hematopoietic stem-progenitor cell towards the stage of CMP and/or before the stage of MEP is stimulated, and the SEQ ID NO is that of mir-16. In one embodiment, the differentiation of a hematopoietic stem-progenitor cell towards the stage of granulocyte is stimulated, and the SEQ ID NO is that of mir-223. In one embodiment, the differentiation of a hematopoietic stem-progenitor cell towards the stage of erythrocyte is stimulated, and the SEQ ID NO is that of mir-221 and/or mir-222.

In one embodiment, a stem-progenitor cell is a human embryonic stem cell (ESC), a neural stem cell or a mesenchymal stem cell, and the SEQ ID NOs are selected from the group consisting of the sequences of let-7b, mir-17, mir-92-1, mir-92-2-p, mir-130a, mir-193, mir-197, mir-212 and mir-222 or the complement thereof.

Also provided herein are methods for stimulating the differentiation of a human embryonic stem cell into an embryonic type (primitive) hematopoietic stem cell. A method may comprise contacting a human embryonic stem cell with a nucleic acid comprising a nucleotide sequence that is at least about 90% identical to that of mir-155. Also provided herein are methods for preventing the differentiation of a human embryonic stem cell into an embryonic type (primitive) hematopoietic stem cell. A method may comprise contacting a human embryonic stem cell with a nucleic acid that is at least about 90% identical to that of the complement of mir-155.

In any of the methods described herein, a stem-progenitor cell may be in vitro, in vivo, ex vivo, isolated from a subject, isolated from the peripheral blood or from the bone marrow. When a cell is in vivo, a method for modulating the differentiation of a stem-progenitor cell that is in a subject may comprise administering to a subject or to a stem-progenitor cell that is in a subject in need thereof one or more nucleic acids, wherein each nucleic acid comprises a nucleotide sequence that is at least about 90% identical to one or more of SEQ ID NOs: 1-34 or the complement thereof. The subject may have a disease that is associated with excessive cell differentiation of a stem-progenitor cell and the method may comprise administering to the subject or to the stem-progenitor cell one or more nucleic acids, wherein each nucleic acid comprises a nucleotide sequence that is at least about 90% identical to one or more of SEQ ID NOs: 1-34. SEQ ID NOs may be selected from the group consisting of those of mir-128a, mir-181a, mir-146, mir-155, mir-24a, mir-17, mir-16, mir-103, mir-107, mir-223, mir-221 and mir-222 or the complement thereof. The subject may have a disease that-is associated with an excessive number of insufficiently differentiated cells and the method may comprise administering to the stem-progenitor cell one or more nucleic acids, wherein each nucleic acid comprises a nucleotide sequence that is at least about 90% identical to one or more of the complement of SEQ ID NOs: 1-34. The disease may be cancer, such as a solid tumor cancer or a blood cancer; the stem-progenitor cell may be a cancer stem cell and the SEQ ID NOs may be selected from the group consisting of the complements of the sequences of mir-128a, mir-181a, mir-146, mir-155, mir-24a, mir-17, mir-16, mir-103, mir-107, mir-223, mir-221 and mir-222 or the complement thereof. A subject in need of treatment may also have low levels of differentiated hematopoietic cells and the method may comprise administering to the stem-progenitor cell one or more nucleic acids, wherein each nucleic acid comprises a nucleotide sequence that is at least about 90% identical to one or more of the complement of SEQ ID NOs: 1-34. The subject may be having or having received chemotherapy or radiation therapy or other therapy destroying one or more types of differentiated cells or the subject may have a disease selected from the group consisting of thrombocytopenia, an immunodeficiency disease, an anemia, leukopenia, granulocytosis or neutropenia.

Also provided herein are methods for increasing the number of red blood cells in a subject, comprising administering to a subject in need thereof, an effective amount a nucleic acid comprising the complement of the nucleotide sequence of mir-16. Other methods provided herein include aA method for treating or preventing leukopenia in a subject, comprising administering to a subject in need thereof, an effective amount of a nucleic acid comprising the complement of mi-146. A subject in need of more differentiated cells may also be a subject infected with human immunodeficiency virus.

Further provided are methods for repopulating a hematopoietic cell population in a subject. A method may comprise obtaining a hematopoietic stem-progenitor cell from a subject, contacting ex vivo the stem-progenitor cell with one or more nucleic acids, wherein each nucleic acid comprises a nucleotide sequence that is at least about 90% identical to one or more of the complements of SEQ ID NOs: 1-33 to thereby obtain differentiated hematopoietic cells; and administering all or a portion of the differentiated hematopoietic cells to the subject. A hematopoietic stem-progenitor cells may be contacted with a nucleic acid comprising the complement of the nucleotide sequence of mir-16.

Also provided are methods for expanding a population of stem-progenitor cells. A method may comprise contacting the stem-progenitor cell with one or more nucleic acids, wherein each nucleic acid comprises a nucleotide sequence that is at least about 90% identical to one or more of SEQ ID NOs: 1-34.

Other embodiments include methods for modulating the expression of a gene that is a target of a miRNA set forth herein, e.g., a gene selected from the group consisting of CXCR4, FOS, FOXP1, GATA3, MYB, RUNX-1, RUNX-3, Spi-8, MED, PU.1, CBPβ, CREBBP, HoxA5, HoxB7, Jun, Meis-1, WWP2, ETS, Evi-1, MYB, AGTR1, AGTR2, GATA2, c-kit, MYB, ETS or PPRyin a cell. A method may comprise administering to the cell one or more nucleic acids, wherein each nucleic acid comprises a nucleotide sequence that is at least about 90% identical to one or more of the SEQ ID NOs: 1-33 or the complement thereof that interacts with the gene, as set forth in Table 1.

Isolated cells and populations of cells are also described herein. In one embodiment, an isolated stem-progenitor cell comprises a heterologous (or recombinant) nucleic acid that is at least about 90% identical to one or more of the SEQ ID NOs: 1-34 or the complement thereof. The stem-progenitor cell may be a stem cell. The isolated stem-progenitor cell may be a hematopoietic cell.

Also provided are compositions and kits, e.g., therapeutic or diagnostic compositions and kits. A composition may comprise one or more nucleic acids, wherein each nucleic acid comprises a nucleotide sequence that is at least about 90% identical to one or more of the SEQ ID NOs: 1-34 or the complement thereof and wherein the nucleic acid is linked to a promoter.

Also provided are methods for determining whether a cell is a stem-progenitor cell. A method may comprise determining the level of one or more miRNAs having one of SEQ ID NOs: 1-34, wherein a level of one or more of these miRNAs that is statistically significantly similar to that of Table 1 or Table 6 indicates that the cell is a stem-progenitor cell. A method for detecting a stem-progenitor cell in a population of cells, may comprise determining the level of one or more miRNAs having one of SEQ ID NOs: 1-34 in a cell in the population of cells, wherein a level of one or more of these miRNAs that is statistically significantly similar to that of Table 1 or 6 indicates the presence of a stem-progenitor cell in the population of cells. A method for determining the proportion of stem-progenitor cells in a population of cells, may comprise determining in a small portion of the population of cells the level of one or more miRNAs having one of SEQ ID NOs: 1-34, and comparing the proportion of cells having one or more of these miRNAs to the total number of cells of the small portion of the population, to thereby derive the proportion of stem-progenitor cells in the population of cells. A method for determining the type of stem-progenitor cell of a hematopoietic stem-progenitor cell may comprise determining the presence of one or more miRNAs that are associated with a particular stage of differentiation, wherein the presence of mir-128a and/or mir-181a indicates that the stem-progenitor cell is a LT-HSC, ST-HSC or an MPP; the presence of mir-146 indicates that the stem-progenitor cell is an MPP that will differentiate into a CLP; the presence of mir-155, mir-24a or mir-17 indicates that the stem-progenitor cell is an MPP that will differentiate into a CMP; the presence of mir-16 indicates that the stem-progenitor cell is a CMP that will differentiate into a MEP; the presence of mir-103 or mir-107 indicates that the stem-progenitor cell with differentiate into a B/Mac or GMP; the presence of mir-223 indicates that the stem-progenitor cell is a GMP that will differentiate into a granulocyte; the presence of mir-221 or mir-222 indicates that the stem-progenitor cell is an MEP that will differentiate into an erythrocyte. A method may further comprise determining the presence of mRNA that is specific for a particular stage of differentiation. For example, the presence of mRNA of one or more of CXCR4, FOS, FOXP1, GATA3, MYB, RUNX-1, RUNX-3, Spi-8, MED or PU.1 indicates that the stem-progenitor cell is a lymphoid stem-progenitor cell; wherein the presence of mRNA of one or more of CBPβ, CREBBP, CXCR4, HoxA5, HoxB7, Jun, Meis-1, RUNX-1, RUNX-3, WWP2, ETS, Evi-1, MYB or PU.1 indicates that the stem-progenitor cell is a myeloid stem-progenitor cell; and wherein the presence of mRNA of one or more of AGTR1, AGTR2, CREBB, FOS, GATA2, GATA3, Jun, c-kit, MYB, RUNX-1, ETS or PPRy indicates that the stem-progenitor cell is an erythroid stem-progenitor cell. Also provided are methods for determining the stemness of a cell. A method may comprise determining the level of one or more miRNAs having a nucleotide sequence of one of let-7b, mir-17, mir-92-1, mir-92-2-p, mir-130a, mir-193, mir-197, mir-212, and mir-222, wherein a level of one or more of these miRNAs that is statistically significantly similar to that of Table 6 indicates that the cell is a stem or stem-progenitor cell.

In still yet a further embodiment, there is provided a method of screening a candidate substance for an effect on cell, e.g., hematopoietic cell, differentiation or proliferation comprising (a) providing a cell that expresses one or more of the miRNAs of SEQ ID NOS: 1-34; (b) contacting said cell with said candidate substance; and (c) assessing the effect of said candidate substance on the expression or stability of one or more of the miRNAs of SEQ ID NOS: 1-34, wherein a candidate substance that modulates the expression or stability of one or more of the miRNAs of SEQ ID NOS: 1-34 is a modulator of cell, e.g., hematopoietic cell, differentiation or proliferation. Assessing may comprise measuring the cellular level or turnover of one or more of the miRNAs of SEQ ID NOS: 1-34.

In another embodiment, there is provided a method of screening a candidate substance for an effect on miRNA-mediated reduction in mRNA or protein translation, comprising (a) providing a cell with a vector encoding a reporter gene fused to a 3′ untranslated region (UTR) sequence, wherein the 3′UTR sequence comprises a nucleotide sequence identical or complimentary to that of an miRNA; (b) contacting the cell with said candidate substance; and (c) assessing the effect of the candidate substance on the expression of the reporter gene. The candidate substance may be one ore more of the miRNAs of SEQ ID NOS: 1-34, or a peptide, protein, nucleic acid, or small molecule. The 3′ UTR may comprise a nucleotide sequence identical or complimentary to that of any miRNA. The 3′ UTR may also comprise a nucleotide sequence from the 3′ UTR of any mRNA. The reporter gene may comprise any known reporter, including luciferase, fluorescent proteins, beta galactosidase, or others.

A method for identifying a gene whose expression modulates the differentiation of a stem-progenitor cell may comprise identifying a gene having a sequence from SEQ ID NO: 1-34 in the 3′ or 5′ UTR, wherein the presence of such as sequence indicates that the gene modulates the differentiation of a stem-progenitor cell. A method for identifying the function of a miRNA may comprise comparing an expression profile of mRNA of a specific cell with that of miRNA of the same cell; comparing the sequences of mRNA and miRNA that are expressed in the cell, wherein the identification of a miRNA having the same as a sequence present in the gene encoding the mRNA indicates that the miRNA is likely to regulate the expression of the gene. A method of screening a candidate substance for an effect on cell differentiation or proliferation, e.g., hematopoietic cell differentiation or proliferation may comprise providing a cell, e.g., a hematopoietic cell, that expresses one or more nucleic acids of SEQ ID NOS: 1-34; contacting said cell with a candidate substance; and assessing the effect of the candidate substance on the expression or stability of one or more nucleic acids of SEQ ID NOS: 1-34. A method for determining whether a miRNA inhibits the expression of a gene may comprise contacting a cell comprising a reporter construct linked to the 3′ or 5′ UTR of the gene with the miRNA, and determining the level of expression of the reporter construct, wherein a lower level of reporter construct in the presence of the miRNA relative to its absence indicates that the miRNA inhibits the expression of the gene.

These and other embodiments of the invention will be better appreciated and understood when considered in conjunction with the following description, exemplification and accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The contents of all cited references (including literature references, issued patents, published patent applications, and GenBank accession numbers as cited throughout this application) are hereby expressly incorporated by reference. In case of conflict, the definitions herein dominate.

### BRIEF DESCRIPTION OF THE DRAWINGS

FIG. 1. A model for HE-miRNA-mediated control of human hematopoiesis. Determination and electronic annotation of the predicted target HSPC-expressed mRNAs for the HE-miRNAs yielded predictions of the differentiation stages where HE-miRNAs might block hematopoietic differentiation. mir-221, mir-222, and mir-223 are predicted to regulate hematopoiesis by the study and supported by other reports; the other miRNAs are new predictions of the present invention. The hierarchy of hematopoietic differentiation is that proposed by Kondo et al. (17). LT-HSC, long-term-repopulating HSC; ST-SHC, short-term-repopulating HSC; MPP, multipotent progenitor cell; CLP, common lymphoid progenitor; CMP, common myeloid progenitor; GMP, granulocyte-macrophage progenitor; MEP, megakaryocyte-erythrocyte progenitor.

FIG. 2. Translation of 16 of 18 tested hematopoietic differentiation-associated mRNAs is controlled by HE-miRNAs. Chimeric mRNAs, consisting of a luc reporter followed by the 3′ UTR from the mRNAs shown, were expressed in K562 cells from a CMV-driven plasmid construct. All values are the percent of luc expression as compared with the control plasmid construct containing luc only. Results are combined from four separate replicate experiments.

FIG. 3. Deletion of specific predicted miRNA-binding sites restored translation of hematopoietic differentiation-associated mRNAs. Individual miRNA-binding sites were deleted from the luc-CXCR4 and luc-KLF2 chimeric mRNA constructs and then transfected into K562 cells. Labels show the mRNA name followed by the miRNA-binding site deleted from that 3′ UTR. All values are the percent of luc expression as compared with the plasmid construct containing luc only. Results are combined from four separate replicate experiments.

FIG. 4. mir-155 inhibited generation of myeloid and erythroid colonies by normal primary human CD34+ cells. Normal PBSC CD34+ cells were transduced with FUGW (empty) or mir-155 lentivector. (A and B) Myeloid (A) and erythroid (B) colonies per 1,000 cells plated. (C) Representative colonies generated by control and mir-155-transduced cells. Results are representative of three separate experiments.

FIG. 5. Production of Luc-3′ UTR reporter constructs and assay for miRNA mediated reduction in mRNA. (A) The luciferase (luc) reporter construct consists of a strong CMV promoter that drives luc expression. 3′ UTR sequences, produced by PCR, were cloned into the 3′ MCS after the luc coding sequence to produce chimeric genes, where luc is under the control of the 3′ UTR from the mRNA predicted to be controlled by a miRNA. (B) If no miRNA binds to the 3′ UTR, luciferase protein is expressed normally. (C) If a miRNA(s), represented as small bars, bind to the 3′ UTR sequence and also inhibit translation, translation will be inhibited resulting in decreased amounts of luciferase protein production.

FIG. 6. Base plasmids used for construction of Luciferase(luc)-3′ UTR Reporter Plasmids. (A) PSP-luc+NF fusion vector. (B) pcDNA-3.1.

FIG. 7. PBSC and BM miRNA expression data. Listed are the probe names for the microRNA chips and the intensity of signal for both PBSC and BM CD34+ cell samples after normalization and merging in GeneSpring.

FIG. 8. miRNAs expressed by PBSC and BM CD34+ cells. Listed are the miRNAs whose expression is shared between both PBSC and BM CD34+ cells, expressed only by PBSC, and expressed only by BM.

FIG. 9. MiR-16 expression changed dramatically during hemin-induced erythroid differentiation of K562 cells, but not during chemically-induced HL60 cell differentiation. (A) MiR levels during hemin-induced erythroid differentiation of K562 cells. (B) HL60 cells were induced to differentiate toward monocytes (1×10−8M 1,25 vitamin D), macrophages (50 ng/ml TPA), eosinophils (5×10−7M butyric acid), or granulocytes (1×10−7M all-trans retinoic acid). Results are a composite of 2 separate experiments containing both biologic and technical duplicates of each sample. (C) Erythroid proteins expressed in control or hemin-induced K562 cells, with our without mir-16 transduction, as measured by SILAC proteomics.

FIG. 10. Enforced miR-16 expression suppressed erythroid differentiation of primary human CD34+ HSPCs. Two days post-transduction of normal CD34+ cells with miR-16-containing or control (FUGW) lentivirus, GFP+ cells were isolated by FACS and assayed by (A) CFC assay or (B) suspension culture. Results in A are means (+/− standard error) of three experiments, and in B are representative plots from three experiments.

FIG. 11 provides the nucleotide sequences of the miRNAs described herein and depicts that some of the miRNAs are encoded by more than one gene.

FIG. 12. miRNA to mRNA compiled interaction chart. This table is a matrix representation of the data outlined in Table 3. Each mRNA transcript is shown in the column direction with each miRNA in the row direction. The mRNA name has been placed under each miRNA that has been predicted to control its translation.

FIG. 13. Cell line miRNA expression data Listed are the probe names for the microRNA chips and the intensity of signal for HAL-01, HL60, K562, KG-1, MV-4-1. REH, TF-1, and TF-1a cell line samples after normalization and merging in GeneSpring.

### DETAILED DESCRIPTION

In certain embodiments, the present invention relates to the role of miRNAs in normal or pathological human cell differentiation, e.g., hematopoiesis. The present inventors have determined the miRNA expression profile of human hematopoietic stem-progenitor cells (HSPCs). The inventors then bioinformaticly combined (i) these miRNA expression data, (ii) mRNA expression data obtained for human CD34+ cells from a previous study by the inventors (8), and (iii) the predicted mRNA targets of all known miRNAs (9-11). Combining these five data sets into one database enabled the in silico examination of the interactions between HSPC-expressed miRNAs (HE-miRNAs) and mRNAs. Based on pairing HE-miRNAs with their putative HSPC-expressed mRNA targets, along with annotation implicating certain of these targets as associated with hematopoietic differentiation, the inventors have preicted which HE-miRNAs control hematopoietic differentiation.

The inventors found that 33 miRNAs (Table 1) are expressed in CD34+ HSPCs obtained from both bone marrow (BM) and mobilized blood peripheral blood stem cell (PBSC) harvests, and demonstrated that translation of several mRNAs associated with hematopoietic differentiation is controlled by these HE-miRNAs. Each of these HE-miRNAs targets multiple hematopoietic differentiation-associated mRNAs, suggesting that a relatively small subset of HE-miRNAs negatively regulates hematopoiesis. Taken together, these observations lea to a proposed model in which many of the protein-coding genes that specify hematopoietic differentiation are expressed at an early time point by undifferentiated HSPCs; yet, these factors appear to be held in check by HE-miRNA-mediated repression of protein translation.

TABLE 1
Levels of the HE-miRNAs expressed in
CD34+ cells from PBSC and BM

| miRNA* | PBSC RFU | BM, RFU |
|---|---|---|
| hsa-let-7b (SEQ ID NO: 1) | 163,441 | 202,826 |
| hsa-mir-10a (SEQ ID NO: 2) | 123,147 | 104,124 |
| hsa-mir-16a (SEQ ID NO: 3) | 123,486 | 86,828 |
| hsa-mir-17 (SEQ ID NO: 4) | 169,601 | 839,361 |
| hsa-mir-20 (SEQ ID NO: 5) | 168,040 | 657,934 |
| hsa-mir-23a (SEQ ID NO: 6) | 445,017 | 896,002 |
| hsa-mir-23b (SEQ ID NO: 7) | 450,110 | 947,757 |
| hsa-mir-24-1 (SEQ ID NO: 8) | 96,912 | 105,926 |
| hsa-mir-25 (SEQ ID NO: 9) | 211,423 | 3,574,408 |
| hsa-mir-26a (SEQ ID NO: 10) | 364,607 | 867,620 |
| hsa-mir-26b (SEQ ID NO: 11) | 368,293 | 2,851,706 |
| hsa-mir-30b (SEQ ID NO: 12) | 136,572 | 91,862 |
| hsa-mir-30d (SEQ ID NO: 13) | 225,309 | 241,004 |
| hsa-mir-92-1 (SEQ ID NO: 14) | 4,115,379 | 5,793,765 |
| hsa-mir-92-2-p (SEQ ID NO: 15) | 3,477,367 | 4,760,875 |
| hsa-mir-93-1 (SEQ ID NO: 16) | 127,149 | 483,003 |
| hsa-mir-103-2 (SEQ ID NO: 17) | 103,209 | 686,012 |
| hsa-mir-103-1 (SEQ ID NO: 18) | 158,154 | 885,352 |
| hsa-mir-106a (SEQ ID NO: 19) | 160,635 | 729,776 |
| hsa-mir-107 (SEQ ID NO: 20) | 136,376 | 832,992 |
| hsa-mir-128a (SEQ ID NO: 21) | 319,848 | 183,657 |
| hsa-mir-130a (SEQ ID NO: 22) | 186,958 | 420,271 |
| hsa-mir-146 (SEQ ID NO: 23) | 626,547 | 444,647 |
| hsa-mir-155 (SEQ ID NO: 24) | 355,623 | 639,819 |
| hsa-mir-181a (SEQ ID NO: 25) | 110,744 | 598,830 |
| hsa-mir-191 (SEQ ID NO: 26) | 247,692 | 3,563,677 |
| hsa-mir-193 (SEQ ID NO: 27) | 104,803 | 481,103 |
| hsa-mir-197 (SEQ ID NO: 28) | 182,695 | 231,027 |
| hsa-mir-213 (SEQ ID NO: 29) | 272,646 | 1,088,714 |
| hsa-mir-221 (SEQ ID NO: 30) | 354,047 | 549,559 |
| hsa-mir-222 (SEQ ID NO: 31) | 354,343 | 200,347 |
| hsa-mir-222-p (SEQ ID NO: 32) | 186,393 | 138,190 |
| hsa-mir-223 (SEQ ID NO: 33) | 2,038,358 | 504,773 |

RFU, relative florescence units.
*All miRNAs listed are the mature form, except those suffixed with “-p” which are the precursor forms (RFU values shown in italics).

The inventors also identified a set of 9 miRNAs that are specifically expressed in any early stem-progenitor cell, e.g., human embryonic stem cells, neurological stem cells and mesenchymal stem cells (see Table 6).

Other examples are further described in the Examplary section.

Nucleic acids are provided related to miRNAs, precursors thereto, and targets thereof. Such nucleic acids may be useful for diagnostic, therapeutic and prognostic purposes, and also for modifying target gene expression and stem cell differentiation. Also provided are methods and compositions that may be useful, among other things, for diagnostic, therapeutic and prognostic purposes. Other aspects of the invention will become apparent to the skilled artisan by the following description of the invention.

### Definitions

Before the present compounds, products and compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

“Animal” as used herein may mean fish, amphibians, reptiles, birds, and mammals, such as mice, rats, rabbits, goats, cats, dogs, cows, apes and humans.

“Biological sample” as used herein may mean a sample of biological tissue or fluid that comprises nucleic acids. Such samples include, but are not limited to, tissue or fluid isolated from animals. Biological samples may also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from animal or patient tissues. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

“Cell” used herein may be a naturally occurring cell or a transformed cell that may contain a vector and may support replication of the vector. Cells may be cultured cells, explants, cells in vivo, and the like. Cells may be prokaryotic cells such as E. coli, or eukaryotic cells such as yeast, insect, amphibian, or mammalian cells, such as CHO and HeLa.

In certain embodiments, a host cell is a hematopoietic cell, for examle, a hematopoietic stem-progenitor cell, a long-term repopulating hematopoietic stem cell, a short-term repopulating hematopoietic stem cell, a multipotent progenitor cell, a common lymphoid progenitor cell, a pro-T cell, a T cell, an NK progenitor cell, an NK cell, a pro-B cell, a B cell, a common myeloid progenitor cell, a B-cell/macrophage bi-potential cell, a granulocyte-macrophage progenitor cell, a megakaryocyte-ertythrocyte progenitor cell, a macrophage, a granulocyte, a megakaryocyte progenitor cell, a megakaryocyte, or an erythrocyte. It is contemplated that the miRNAs described here may play a role in the differentiation pathways of other cells. The cell may also be a bacterial, fungal, plant, insect or other type of animal cell.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand, unless the nucleic acid is a single stranded nucleic acid. Many variants of a nucleic acid may be used for the same purpose as a given nucleic acid. A single strand provides a probe that may hybridize to a target sequence under stringent hybridization conditions. Thus, a nucleic acid may also be a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

“Operably linked” used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Selectable marker” used herein may mean any gene which confers a phenotype on a host cell in which it is expressed to facilitate the identification and/or selection of cells which are transfected or transformed with a genetic construct. Representative examples of selectable markers include the ampicillin-resistance gene (Amp^{r}), tetracycline-resistance gene (Tc^{r}), bacterial kanamycin-resistance gene (Kan^{r}), zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene.

“Stringent hybridization conditions” used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30.degree. C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5.times.SSC, and 1% SDS, incubating at 42° C., or, 5×SSC, 1% SDS, incubating at 65° C., with wash in 0.2×SSC, and 0.1% SDS at 65° C.

“Substantially complementary” used herein may mean that a first sequence is at least about 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least about 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Variant” used herein to refer to a nucleic acid may mean (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Vector” is used to refer to a carrier nucleic acid molecule into which a nucleic acid sequence can be inserted for introduction into a cell where it can be replicated. In accordance with the present invention, it may be desirable to express the miRNAs of the present invention in a vector. The term “exogenous,” means that the vector or entity referred to is foreign to the cell into which it is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., BACs, YACs). One of skill in the art would be well equipped to construct a vector through standard recombinant techniques (see, for example, Maniatis et al., 1988 and Ausubel et al., 1994, both incorporated herein by reference).

“Expression vector” refers to any type of genetic construct comprising a nucleic acid coding for an RNA capable of being transcribed. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A “promoter” may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the beta-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

In certain embodiments, it may be useful to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. (2001), incorporated herein by reference). The promoters employed may be constitutive, tissue-specific, inducible, and/or useful under the appropriate conditions to direct high level expression of the introduced DNA segment. The promoter may be heterologous or endogenous.

Additionally any promoter/enhancer combination (as per, for example, the Eukaryotic Promoter Data Base EPDB, www.epd.isb-sib.ch) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Surface markers of hematopoietic cells are as follows: HSCs (CD34+/CD38−/CD90+/Lin−), CMPs (CD34+/CD38+/CD123+/CD45RA−), and MEPs (CD34+/CD38+/CD123−/CD45RA−). HSPCs may be CD34+; HSC-enriched cells (CD34+/[CD38/Lin]lo); HPC-enriched cells (CD34+/[CD38/Lin]hi); Erythroid progenitor-enriched cells (CD34+/[CD71/CD235A]+); and (e) Erythroid precursor/mature cells (CD34−/[CD71/CD235A]+).

### MicroRNA

While not being bound by theory, a gene coding for an miRNA may be transcribed leading to production of an miRNA precursor known as the pri-miRNA. The pri-miRNA may be part of a polycistronic RNA comprising multiple pri-miRNAs. The pri-miRNA may form a hairpin with a stem and loop. The stem may comprise mismatched bases.

The hairpin structure of the pri-miRNA may be recognized by Drosha, which is an RNase III endonuclease. Drosha may recognize terminal loops in the pri-miRNA and cleave approximately two helical turns into the stem to produce a 60-70 nt precursor known as the pre-miRNA. Drosha may cleave the pri-miRNA with a staggered cut typical of RNase III endonucleases yielding a pre-miRNA stem loop with a 5′ phosphate and about a 2 nucleotide 3′ overhang. Approximately one helical turn of the stem (about 10 nucleotides) extending beyond the Drosha cleavage site may be essential for efficient processing. The pre-miRNA may then be actively transported from the nucleus to the cytoplasm by Ran-GTP and the export receptor Ex-portin-5.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and about a 2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specifity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-8 of the miRNA. A case has been reported in animals where the interaction between the miRNA and its target was along the entire length of the miRNA. This was shown for mir-196 and Hox B8 and it was further shown that mir-196 mediates the cleavage of the Hox B8 mRNA (Yekta et al., 2004, Science 304-594). Such interactions are also known only in plants (Bartel & Bartel, 2003, Plant Physiol 132-709).

A number of studies have examined the base-pairing requirement between miRNA and its mRNA target for achieving efficient inhibition of translation (reviewed by Bartel, 2004, Cell, 116-281). In mammalian cells, the first 8 nucleotides of the miRNA may be important (Doench & Sharp, 2004, GenesDev, 2004-504). However, other parts of the miRNA may also participate in mRNA binding. Moreover, sufficient base pairing at the 3′ can compensate for insufficient pairing at the 5′ (Brennecke et al., 2005, PLoS 3-e85). Computational studies analyzing miRNA binding on whole genomes have suggested a specific role for bases 2-7 at the 5′ of the miRNA in target binding but a role for the first nucleotide, usually “A”, was also recognized (Lewis et al., 2005, Cell, 120-15). Similarly, nucleotides 1-7 or 2-8 were used to identify and validate targets by Krek et al. (2005, Nat Genet, 37-495).

The target sites in the mRNA may be in the 5′ UTR, the 3′ UTR or within the coding region. Multiple miRNAs may regulate the same mRNA target by recognizing the same or multiple sites. The presence of multiple miRNA binding sites in most genetically identified targets may indicate that the cooperative action of multiple RISCs provides the most efficient translational inhibition. Similarly, a single miRNA may regulate multiple mRNA targets by recognizing the same or similar sites on the different targets.

Without being bound by theory, miRNAs may direct the RISC to downregulate gene expression by several mechanisms: mRNA cleavage, translational repression, or chromatin remodeling. The miRNA may specify cleavage of the mRNA if the miRNA has a certain degree of complementarity to the mRNA. When an miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

### Methods for Modulating Differentiation of Stem-Progenitor Cells

Provided herein are methods for modulating the differentiation of an incompletely differentiated cell, i.e., a cell that is capable of further differentiation, e.g., a stem-progenitor cell (SPC). A “stem-progenitor cell” is any cell that is capable of further differentiation and/or is capable of proliferation, i.e., undergoing cell division (i.e. renew itself). An SPC may be a eukaryotic cell, e.g., a mammalian cell, a vertebrate cell, a human cell or a non-human cell, e.g., a non-human animal cell. A SPC may be an embryonic SPC, e.g., a human embryonic stem cell, an adult stem cell, as well as a cancer stem cell. It may be also be an adult SPC, and may be of any cell lineage, e.g., a cell lineage specific stem cell, e.g., a neural, mesenchymal, hematopoietic, muscle, stromal cells (e.g., bone marrow stromal cells), skin, liver, brain, and blood vessel specific stem cells.

The potency of a cell is sometimes used to specify the differentiation potential (the potential to differentiate into different cell types) of a stem or stem-progenitor cell. Totipotent stem cells are sometimes referred to as cells that are produced from the fusion of an egg and sperm cell. Cells produced by the first few divisions of the fertilized egg may also be totipotent. These cells can differentiate into embryonic and extraembryonic cell types. Pluripotent stem are sometimes referred to as cells that are the descendants of totipotent cells and can differentiate into cells derived from any of the three germ layers. Multipotent stem cells are cells that can produce only cells of a closely related family of cells (e.g. hematopoietic stem cells differentiate into red blood cells, white blood cells, platelets, etc.). Unipotent cells can produce only one cell type, but have the property of self-renewal which distinguishes them from non-stem cells (e.g. muscle stem cells).

Hematopoietic stem cells may give rise to all the types of blood cells: red blood cells, B lymphocytes, T lymphocytes, natural killer cells, neutrophils, basophils, eosinophils, monocytes, macrophages, and platelets. Bone marrow stromal cells (mesenchymal stem cells) may give rise to a variety of cell types: bone cells (osteocytes), cartilage cells (chondrocytes), fat cells (adipocytes), and other kinds of connective tissue cells such as those in tendons. Neural stem cells in the brain may give rise to its three major cell types: nerve cells (neurons) and two categories of non-neuronal cells—astrocytes and oligodendrocytes. Epithelial stem cells in the lining of the digestive tract occur in deep crypts and give rise to several cell types: absorptive cells, goblet cells, Paneth cells, and enteroendocrine cells. Skin stem cells occur in the basal layer of the epidermis and at the base of hair follicles. The epidermal stem cells amy give rise to keratinocytes, which migrate to the surface of the skin and form a protective layer. The follicular stem cells can give rise to both the hair follicle and to the epidermis (see, e.g., stemcells.nih.gov.).

Certain adult stem cell types are pluripotent. This ability to differentiate into multiple cell types is called plasticity or transdifferentiation. The following list offers examples of adult stem cell plasticity. Hematopoietic stem cells may differentiate into: three major types of brain cells (neurons, oligodendrocytes, and astrocytes); skeletal muscle cells; cardiac muscle cells; and liver cells. Bone marrow stromal cells may differentiate into: cardiac muscle cells and skeletal muscle cells. Brain stem cells may differentiate into: blood cells and skeletal muscle cells (see, e.g., stemcells.nih.gov.).

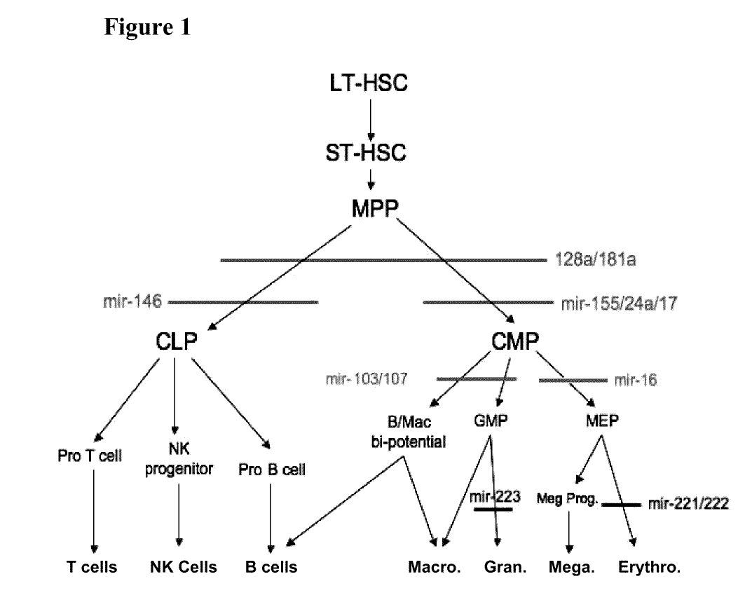

In one embodiment, a stem-progenitor cell is a hematopoietic stem-progenitor cell (HSPC). An HSPC is any cell that is not fully differentiated, i.e., that is capable of further differentiation and/or is capable of further proliferation (or cell division). An HSPC may be any cell that is set forth in FIG. 1, provided that it is not a fully differentiated cell.

A method may comprise contacting a hematopoietic stem-progenitor cell with one or more agents that modulate the level or activity of exactly or about 1, 2, 3, 5, 10, 15, 20, 25, 30, 32, or 33 miRNAs having one of SEQ ID NOs: 1-33 (see Table 1). In one embodiment, an agent that modulates the level or activity of a miRNA is a nucleic acid, e.g., a natural, synthetic or recombinant nucleic acid, comprising a nucleotide sequence that is at least about 50%, 60%, 70%, 80%, 85%, 90%, 95%, 96%, 97%, 98%, 99% or 100% to that of the miRNA, pre-miRNA or pri-miRNA or the complement thereof. When referring to “comprising a sequence” herein, it is understood that in certain embodiments, “consisting essentially of” or “consisting of” are also included. For example, in certain embodiments, a nucleic acid comprises, consists essentially of or consists of a nucleotide sequence that is at least about 50%, 60%, 70%, 80%, 85%, 90%, 95%, 96%, 97%, 98%, 99% or 100% to that of the miRNA, pre-miRNA or pri-miRNA or the complement thereof. In certain embodiments, a nucleic acid comprises a nucleotide sequence that differs (by substitution, addition or deletion) from that of a miRNA, pre-miRNA or pri-miRNA in about 1, 2, 3, 4, 5, 6, 7, 8, 9, 10 or more nucleotides. In embodiments in which it is desired to inhibit the differentiation of a stem-progenitor cell, a nucleic acid comprising a nucleotide sequence that is identical to or homologous to that of all or a portion of the miRNA, pre-miRNA or pri-miRNA may be used. In embodiments in which it is desired to stimulate the differentiation of a stem-progenitor cell, a nucleic acid comprising a nucleotide sequence that is identical to or homologous to that of the complement of all or a portion of the miRNA, pre-miRNA or pri-miRNA may be used. Generally, when referring to targeting, modulating, inhibiting or stimulating a “miRNA,” it is understood that this may include affecting related or derivative molecules of the miRNA, such as pre- and pri-miRNAs. A difference in nucleotide sequence is permitted provided that i) if the nucleic acid is an antagonist of the miRNA, the nucleic acid binds (or hybridizes) sufficiently strongly and/or specifically to the miRNA, pre-miRNA or pri-miRNA to thereby inhibit or reduce its activity and ii) if the nucleic acid is an agonist of the miRNA, the nucleic acid mimics or has the biological activity or a significant portion thereof, of the wild-type miRNA.

Nucleic acids for modulating the level or activity of miRNAs may, in addition to a sequence that is identical or homologous.to that of the miRNA or complement thereof, also comprise additional, unrelated nucleotides, provided that they do not interfere with the mimicking or inhibition, respectively, of the miRNA. They may comprise, e.g., about 1, 2, 3, 4, 5, 6, 7, 8, 9, 10 or more nucleotides that are located either 5′ and/or 3′ (or internal) to the sequence for modulating the level or activity of the miRNA.

An agonist of a miRNA (understood herein to also include agonists of the related molecules, e.g., pre- or pri-miRNA) is any agent, e.g., a nucleic acid comprising a nucleotide sequence that is identical or homologous to that of the miRNA, that mimics the action of the miRNA. An antagonist of a miRNA (understood herein to also include agonists of the related molecules, e.g., pre- or pri-miRNA) is any agent, e.g., a nucleic acid comprising a nucleotide sequence that is identical or homologous to that of the complement of the miRNA, that inhibits the action of the miRNA.

In certain embodiments, a nucleic acid comprises at least about 2, 3, 5, 10, 15, 20, 25, 30, or more nucleotide sequences that are identical or homologous to that of a miRNA or the complement thereof. The nucleotide sequences may be mimicking or inhibiting the level or activity of one miRNA or alternatively of at least about 2, 3, 5, 10, 15, 20, 25, 30, 32, 33, or more different miRNAs, e.g., those having SEQ ID NOs: 1-33.

Exemplary steps of hematopoiesis that may be modulated by specific miRNAs are set forth in FIG. 1. Generally, agonists of specific miRNAs will inhibit (or block) the differentiation of a hematopoietic stem-progenitor cell past a certain stage of differentiation and/or maintain the cell at a certain stage of differentiation, while antagonists of specific miRNAs will stimulate the differentiation of the cell towards or past (or beyond) a specific stage of differentiation. Thus, the methods described herein allow one to block a cell early in differentiation or later in differentiation, depending on which miRNA agonist is used. The methods described herein also allow one to stimulate the differentiation of an early progenitor or stem cell or a cell at a later stage of diffentiation.

In certain embodiments, a hematopoietic stem-progenitor cell is a pluripotent stem-progenitor cell, a totipotent stem-progenitor cell, a long-term repopulating hematopoietic stem-progenitor cell (HSPC), a short term repopulating HSPC, or a multipotent stem-progenitor cell and its differentiation is modulated with one or more nucleic acids comprising one or more SEQ ID NOs selected from the group consisting of those of mir-128a, mir-181a, mir-146, mir-155, mir-24a, and mir-17 or the complement thereof. In certain embodiments, the differentiation of a hematopoietic stem-progenitor cell into a common lymphoid stem-progenitor is modulated with one or more nucleic acids comprising one or more SEQ ID NOs of mir-146 or the complement thereof. In certain embodiments, the differentiation of a hematopoietic stem-progenitor cell into a common myeloid stem-progenitor is modulated with one or more nucleic acids comprising one or more SEQ ID NOs selected from the group consisting of those of mir-155, mir-24a or mir-17 or the complement thereof. In certain embodiments, the differentiation of a hematopoietic stem-progenitor cell that is a common myeloid stem-progenitor cell into macrophages and granulocytes is modulated with one or more nucleic acids comprising one or more SEQ ID NOs of those of mir-103 or mir-107 or the complement thereof In certain embodiments, the differentiation of a hematopoietic stem-progenitor cell that is a common myeloid stem-progenitor cell into megakaryocyte-erythrocyte stem-progenitor is modulated with one or more nucleic acids comprising one or more SEQ ID NOs of mir-16 or the complement thereof. In certain embodiments, the differentiation of a hematopoietic stem-progenitor cell that is a granulocyte-macrophage stem-progenitor into a granulocyte is modulated with one or more nucleic acids comprising one or more of the SEQ ID NO of mir-223 or the complement thereof. In certain embodiments, the differentiation of a hematopoietic stem-progenitor cell that is a megakaryocyte-erythrocyte stem-progenitor cell into erythrocytes is modulated with one or more nucleic acids comprising one or more of the SEQ ID NOs of mir-221 and/or mir-222 or the complement thereof.

Also provided are methods for stimulating the differentiation of human embryonic stem cells into embryonic type (primitive) hematopoietic stem cells. A method may comprise contacting the human embryonic stem cells with an agonist of mir-155, e.g., a nucleic acid comprising a nucleotide sequence that is at least about 90% identical to that of mir-155. Another method prevents the differentiation of human embryonic stem cells into embryonic type (primitive) hematopoietic stem cells, e.g., by contacting the human embryonic stem cells with an antagonist of mir-155, e.g., a nucleic acid that is at least about 90% identical to that of the complement of mir-155.

In certain embodiments, the differentiation of a HSPC is modulated with the use of more than one agent. For example, the differentiation of a HSPC may be modulated with the use of one or more agents that target the level or activity of at least about 2, 3, 4, 5, 10, 15, 20, 25, 30, 31, 32 or 33 different miRNAs having one of SEQ ID NOs: 1-33. In one embodiment, the differentiation of a HSPC is modulated by contacting or introducing into the HSPC an agonist or antagonist of any one of the miRNAs having one of SEQ ID NOs: 1-33. In one embodiment, the differentiation of a HSPC is stimulated by contacting or introducing into the HSPC an agonist or antagonist of any one of the miRNAs set forth in FIG. 1, i.e., mir-128a, mir-181a, mir-146, mir-155, mir-24a, mir-17, mir-16, mir-103, mir-107, mir-223, mir-221 and mir-222. In situations in which one may wish to differentiate an HSPC into a cell at a certain stage of differentiation (or lineage), one may include a combination of agonists of certain miRNAs and antagonists of other miRNAs. For example, to obtain a population of cells enriched in CMPs, one may contact or administer into a HSPC an agonist of mir-103 and/or mir-107, an agonist of mir-16, an agonist of mir-146 and an antagonist of mir-128a, mir-181a, mir-155, mir-24a and/or mir-17. Other combinations of agonists and antagonists will be evident from FIG. 1.

Other methods provided herein modulate the differentiation of non-hematopoietic stem-progenitor cells, e.g., neuronal, mesenchymal and embryonic stem cell (see Table 6). Modulating the differentiation of such cells may be made with agonists and/or antagonists of miRNAs of Table 6, as described in the context of hematopoietic stem cells.

In certain embodiments (which may relate to methods of differentiation or other methods described herein, miRNAs are not specific miRNAs. For example, in certain embodiments, a miRNA is not mi-146, 181, 221, 222 or 223.

### Methods for Enriching or Expanding a Population of Stem-Progenitor Cells

Methods of modulating the differentiation of SPCs may be used to enrich populations of cells in one or more specific types of cells or cells of particular lineages or for expanding populations of cells. For example, a method may be used to expand a population of undifferentiated cells, e.g., stem or progenitor or stem-progenitor cells, such as by blocking differentiation, e.g., by using agonists of miRNAs described herein. In one embodiment, the initial population of cell is a population of cells comprising undifferentiated cells and the method comprises contacting the initial population of cells with one or more agonist of a miRNA set forth in Table 1. In one embodiment, the initial population of cells comprises hematopoietic cells, e.g., from the peripheral blood or bone marrow, and the initial population of cells is contacted with one or more agonists having a sequence from SEQ ID NO: 1-33, such as to enrich the population of cells in hematopoietic stem-progenitor cells that are undifferentiated or at an early stage of differentiation. In other embodiments, a population of cells may be enriched in specific types of hematopoietic cells by using agonists, antagonists of miRNAs of Table 1, e.g., having one or SEQ ID NO: 33 or a combination of agonists and antagonists. A method may be used for enriching a population of cells into differentiated cells, partially differentiated cells or undifferentiated cells.

Enriching or expanding a population of cells may be conducted in vitro, ex vivo or in vivo. When expanding a population of cells in vitro, the initial population of cells may be from a cell line, including embryonic or cancer stem cell lines, or from a subject. An initial population of cells may comprise multiple cell types, or alternatively, it may consist essentially of only one cell type. Where an initial population of cells comprises different types of cells, it may first be enriched in certain types of cells by using techniques known in the art, by immunoaffinity. Accordingly, an initial population of cells may be population of cells that is enriched in certain types of cells. In one example, an initial population of cells are cells obtained from the peripheral blood or bone marrow of a subject and the population of cells has been enriched in CD34+ cells.

In one embodiment, an initial population of cells is obtained from a subject; the initial population of cells is enriched or expanded into one or more particular cell types to obtain an enriched or expanded population of cells; and the enriched or expanded population of cells is administered to a subject, which may be the same subject from whom the initial population of cells was obtained or a different subject. The cells may also be additionally purified, enriched or expanded by other methods, e.g., based on the presence of certain surface markers or by FACS.

In one embodiment, a population of cells is enriched or expanded in vivo. A method may comprise administering to a subject in need thereof a therapeutically effective amount of one or more agonists or antagonists of one or more miRNAs set forth in Table 1, e.g., those having a sequence of SEQ ID NOs: 1-33, to thereby enrich or expand a population of cells in the subject. An agent may be administered locally of systemically. In one embodiment, for expanding or enriching hematopoietic cells in certain types of cells, an agent is administered into the bone marrow. For enriching or expanding a population of cells in the blood, an agent may be administered directly into the blood. An agent may comprise or be comprised in a delivery vehicle that allows targeting to particular cell types. Such targeting may be based on cell surface molecules of the target population of cells.

As it has been shown herein, mir-155 prevents further differentiation of a very early hematopoietic stem cell (HSC), e.g., when differentiated from an embryonic stem cell. In one embodiment, a method for expanding a population of HSC or produce HSC from embryonic stem cells, comprises contacting the cells with an agonist of mir-155. Also provided are methods for stimulating the differentiation of embryonic stem cells past HSCs, comprising contacting the cells with an antagonist of mir-155.

Based at least on the identification of 9 miRNAs that are expressed in several types of stem cells, also encompassed are methods for expanding a population of stem cells, e.g., embryonic stem cells, e.g., human embryonic stem cells. A method may comprise contacting a stem cell with one or more agonists of one or more miRNAs selected from the group consisting of let-7b, mir-17, mir-92-1, mir-92-2p, mir-130a, mir-193, mir-197, mir-212 and mir-222 (“stem cell miRNAs”; Table 6), to thereby prevent the differentiation of the stem cells. Also provided are methods for stimulating the differentiation of a population of stem cells. A method may comprise contacting a stem cell with one or more antagonists of one or more miRNAs selected from the group consisting of let-7b, mir-17, mir-92-1, mir-92-2p, mir-130a, mir-193, mir-197, mir-212 and mir-222, to thereby stimulate the differentiation of the stem cells.

Also provided herein are isolated, purified, expanded and/or enriched populations of cells that are obtained using one or more of the methods described herein for modulating the differentiation of cells. A population of cells may at least about 50%, 60%, 70%, 80%, 90%, 95%, 97%, 98%, 99% or 100% enriched in one or more type or lineage of cells. Populations of cells may also comprise cells comprising one or more heterologous nucleic acid encoding or identical to one or more miRNAs, such as those further described herein. Populations of cells may also be comprised in a pharmaceutical composition, e.g., for administration of the cells to a subject, e.g., in stem cell therapy. Populations of cells and compositions comprising such may also be present in a delivery vehicle, such as a liposome, a viral particle or a nanoparticle, e.g., as further described herein.

Expanded populations of early stem progenitor cells may be used for the production of cells oe one or more lineage, which may then be transplanted into a subject. Alternatively, expanded populations of cells, e.g., undifferentiated cells, are administered to a subject, e.g., for stem cell therapy.

### Nucleic Acids Controlling Stem-Progenitor Cell Differentiation

Nucleic acids controlling stem progenitor cell, e.g., hematopoietic stem-progenitor, cell differentiation are provided herein. The nucleic acid may comprise the sequence of SEQ ID NOS: 1-34 or variants thereof. The variant may be a complement of the referenced nucleotide sequence. The variant may also be a nucleotide sequence that is substantially identical to the referenced nucleotide sequence or the complement thereof. The variant may also be a nucleotide sequence that hybridizes under stringent conditions to the referenced nucleotide sequence, complements thereof, or nucleotide sequences substantially identical thereto.

The nucleic acid may have a length of from 10 to 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single stranded molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference. In certain embodiments, it may be useful to incorporate the nucleic acids into a vector, as described supra.

### Pri-miRNA

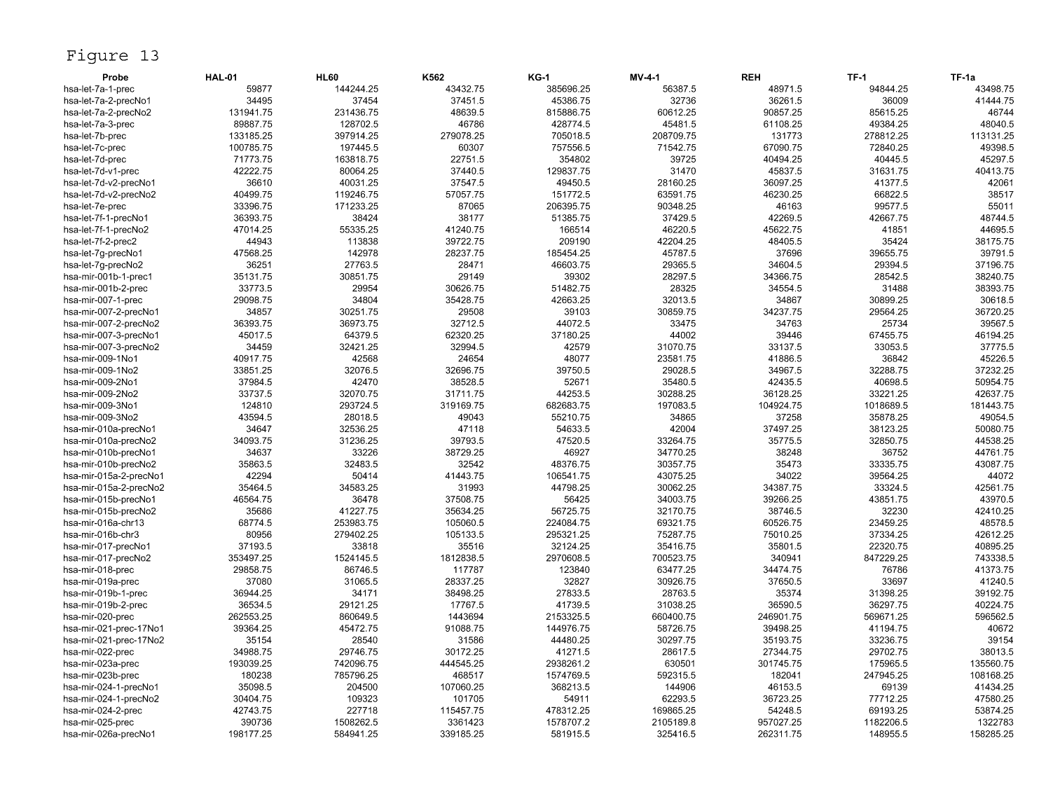

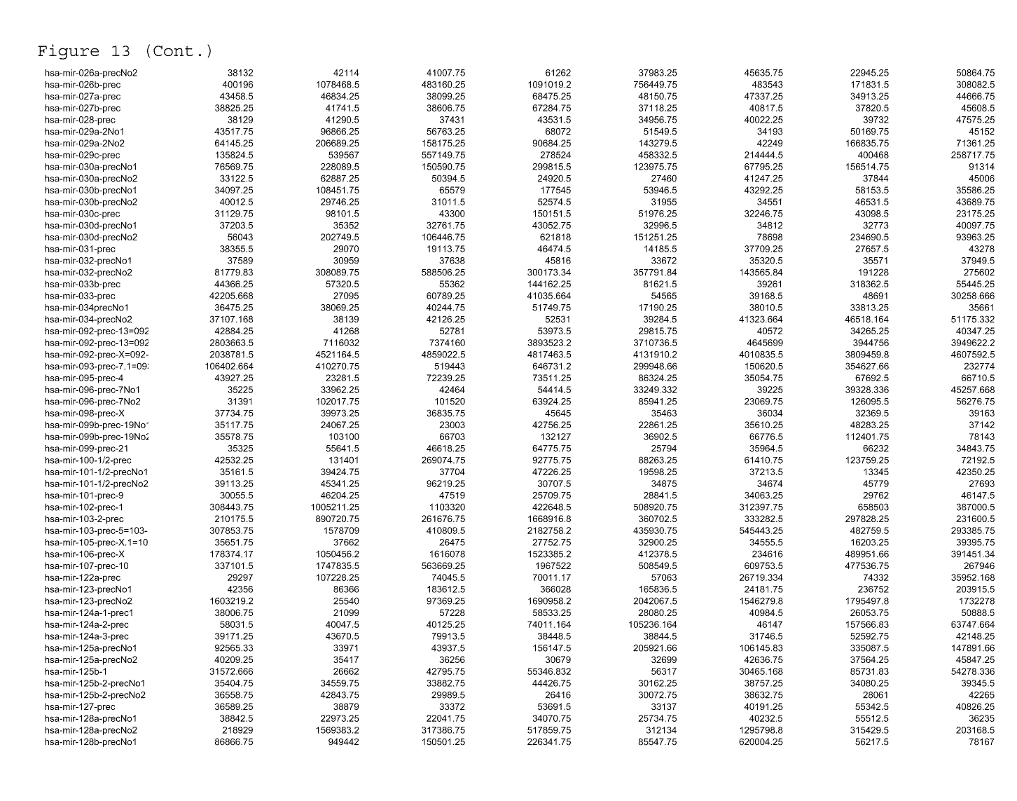

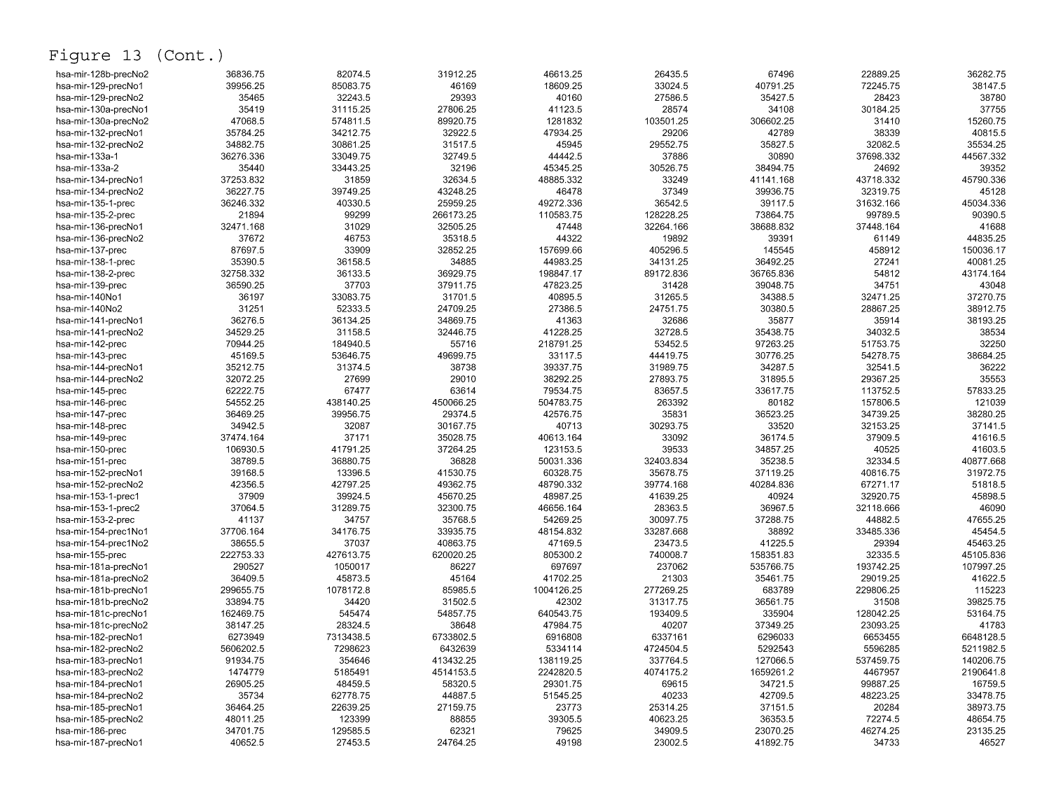

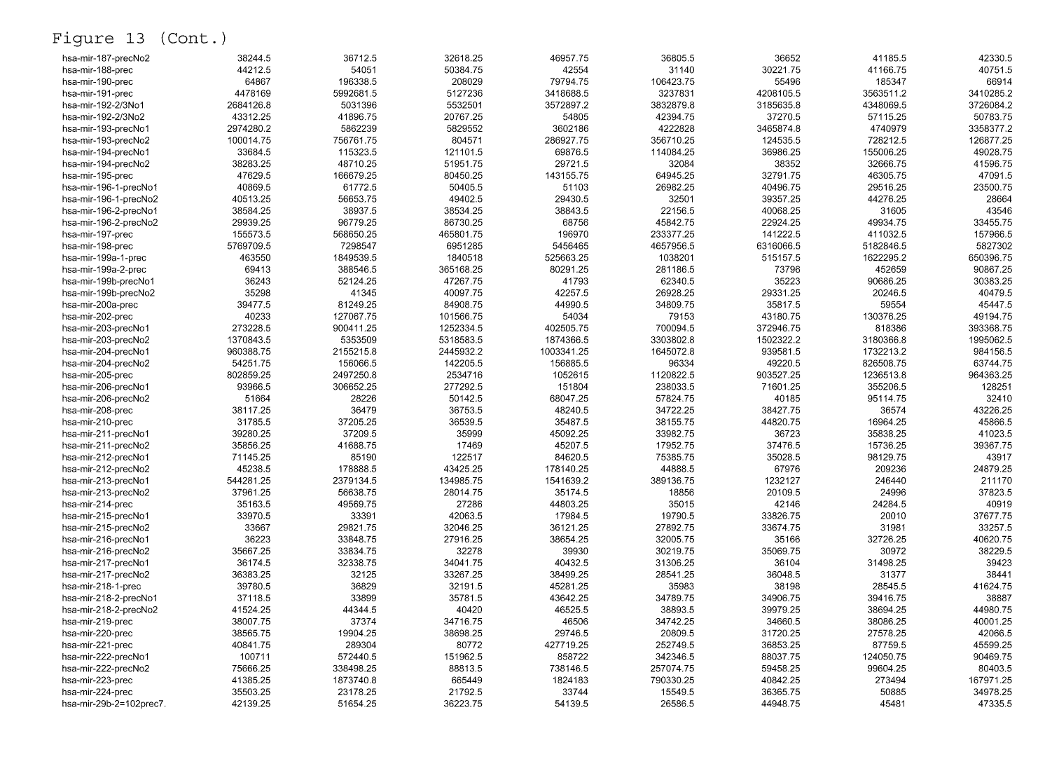

The nucleic acid may comprise a sequence of a pri-miRNA or a variant thereof. The pri-miRNA sequence may comprise from 45-250, 55-200, 70-150 or 80-100 nucleotides. The sequence of the pri-miRNA may comprise a pre-miRNA, miRNA and miRNA*, as set forth herein, and variants thereof. The sequence of the pri-miRNA may comprise the sequence of SEQ ID NOS:1-34, the complement of an miRNA binding site referred to on an mRNA in FIG. 13, or variants thereof.

The pri-miRNA may form a hairpin structure. The hairpin may comprise a first and second nucleic acid sequence that are substantially complementary. The first and second nucleic acid sequence may be from 37-50 nucleotides. The first and second nucleic acid sequence may be separated by a third sequence of from 8-12 nucleotides. The hairpin structure may have a free energy less than −25 Kcal/mole as calculated by the Vienna algorithm with default parameters, as described in Hofacker et al., Monatshefte f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

### Pre-miRNA

The nucleic acid may also comprise a sequence of a pre-miRNA or a variant thereof. The pre-miRNA sequence may comprise from 45-90, 60-80 or 60-70 nucleotides. The sequence of the pre-miRNA may comprise a miRNA and a miRNA* as set forth herein, The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-34, the complement of an miRNA binding site on an mRNA referred to in FIG. 13, or variants thereof.

miRNA

The nucleic acid may also comprise a sequence of an miRNA (including miRNA*) or a variant thereof The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1-34, the complement of an miRNA binding site on an mRNA referred to in FIG. 13, or variants thereof.

### Anti-miRNA

The nucleic acid may also comprise a sequence of an anti-miRNA that is capable of blocking the activity of a miRNA or miRNA*, such as by binding to the pri-miRNA, pre-miRNA, miRNA or miRNA* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially complementary to the 5′ of a miRNA and at least 5-12 nucleotides that are substantially identical to the flanking regions of the target site from the 5′ end of the miRNA, for the purposes of binding to a miRNA and repressing its activity; or (b) at least 5-12 nucleotides that are substantially identical to the 3′ of a miRNA and at least 5 nucleotide that are substantially complementary to the flanking region of the target site from the 3′ end of the miRNA, for the purposes of inhibiting the ability of a miRNA to bind to its target. The sequence of the anti-miRNA may comprise the complement of SEQ ID NOS:1-34, the miRNA binding site on an mRNA referred to in FIG. 13, or variants thereof.

In some embodiments, morpholino-based oligonucleotides may be used to block the activity of an miRNA. Morpholinos comprise standard nucleic acid bases bound to morpholine rings, instead of deoxyribose rings, and linked through phosphorodiamidate groups, instead of phosphates (Summerton and Weller, 1997, Antisense & Nucleic Acid Drug Development, 7: 187-195). In other embodiments, “multi-blocking morpholinos,” which may inhibit the activity of a targeted miRNA by blocking several steps of its maturation, may be used.

### Vectors

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with beta-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

### Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

Adenoviral Vectors. A particular method for delivery of nucleic acids involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

AAV Vectors. The nucleic acid may be introduced into a cell using adenovirus-assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Retroviral Vectors. Retroviruses have promise as delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other Viral Vectors. Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

Delivery Using Modified Viruses. A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### Delivering Nucleic Acids to Cells

Suitable methods for nucleic acid delivery to cells for use with the current invention are believed to include virtually any method by which a nucleic acid (e.g., DNA), as known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al, 1987); by liposome-mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by desiccation/inhibition-mediated DNA uptake (Potrykus et al., 1985), and any combination of such methods.

Methods for tranfecting cells and tissues removed from an organism in an ex vivo setting are known to those of skill in the art. For example, cannine endothelial cells have been genetically altered by retrovial gene tranfer in vitro and transplanted into a canine (Wilson et al., 1989). In another example, yucatan minipig endothelial cells were tranfected by retrovirus in vitro and transplated into an artery using a double-ballonw catheter (Nabel et al., 1989). Thus, it is contemplated that cells or tissues may be removed and tranfected ex vivo using the nucleic acids of the present invention. In some embodiments, the transplanted cells or tissues may be placed into an organism. In some embodiments, a nucleic acid is expressed in the transplated cells or tissues.

Injection. In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of nucleic acid used may vary upon the nature of the antigen as well as the organelle, cell, tissue or organism used

Electroporation. In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

Calcium Phosphate. In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran. In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading. Additional embodiments of the present invention include the introduction of a nucleic acid by direct sonic loading. LTK.sup.-fibroblasts have been transfected with the thymidine kinase gene by sonication loading (Fechheimer et al., 1987).

Liposome-Mediated Transfection. In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor Mediated Transfection. Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

### Synthetic Gene

A synthetic gene is also provided comprising a nucleic acid described herein operably linked to a transcriptional and/or translational regulatory sequence. The synthetic gene may be capable of modifying the expression of a target gene with a binding site for a nucleic acid described herein. Expression of the target gene may be modified in a cell, tissue or organ. The synthetic gene may be synthesized or derived from naturally-occurring genes by standard recombinant techniques. The synthetic gene may also comprise terminators at the 3′-end of the transcriptional unit of the synthetic gene sequence. The synthetic gene may also comprise a selectable marker.

### Biochip

A biochip is also provided. In some embodiments, the biochip may be specific for the determination of the state of a hematopoietic cell. The biochip may comprise a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

Expression Analysis/detecion of miRNAs

Also provided herein a methods of detecting and/or quantifying stem-progenitor cells. As further described in the Examples, the inventors have identified a “molecular signature” of all essentially undifferentiated stem cells and a “molecular signature” of hematopoietic stem-progenitor cells. As understood in the art, a “molecular signature” refers to expression levels of a certain number of biological molecules, in the instant case, of miRNAs. Thus, the finding of such a signature will be evidence of the presence of stem-progenitor cells. Accordingly, a method may comprise determining the level of one or more stem cell of HSPC miRNAs and comparing that level to that of the levels set forth in Table 1 for HSPCs and in Table 6 for essentially undifferentiated stem cells. Such methods may be used to detect or quantify the presence or number of undifferentiated stem cells or HSPCs. In one embodiment, a sample of cells is obtained from a subject and the level of miRNAs is determined. A sample may be a sample of blood cells or of bone marrow. A method may also comprise detecting one or more mRNA that is expressed specifically in hematopoietic stem-progenitor cells, e.g., those described in US application publication No. 22070036765.

A method of detection may comprise determining the level of 1, 2, 3, 4, 5, 10, 15, 20, 25, 30, 31, 32 or 33 miRNAs of Table 1 or 1, 2, 3, 4, 5, 6, 7, 8 or 9 miRNAs of Table 6.

A detection method described herein may also be used to determine the degree of enrichment of a population of cells that is being enriched in particular cell types.

Comparing a molecular fingerprint or signature where an insignificant, e.g., statistically insignificant difference between a miRNA expression profile of a population of cells and that of the molecular signature or portion thereof set forth in Table 1 or 6 or elsewhere herein indicates that the population of cells are significantly the same. A fingerprint may be the level of expression of all the miRNA of a Table set forth herein, e.g., Table 1 or Table 6, or it can be that of a portion of the miRNAs, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 44 miRNAs.

A method may comprise obtaining a sample from a subject, isolating a population or subpopulation of cells therefrom; detecting the level of miRNAs of a table, e.g., Table 1 or 6; and comparing that or those levels of expression with the molecular signature or portion thereof set forth in the table. In one embodiment, blood cells, e.g., PBMCs or bone marrow cells are obtained from a subject. In one embodiment, CD34+ cells are isolated from the population of cells and the level of expression of one or more miRNAs set forth in Table 1 is determined and compared to that in Table 1. In one embodiment, the level of expression of the 33 mirNAs set forth in Table 1 is determined in CD34+ cells and their level is compared with the molecular signature set forth in Table 1. A statistically significant difference in the level of one or more miRNAs may be indicative of an abormality of CD34+ cells or hematopoietic stem-progenitor cells in the subject. Abnormal may be an abnormal number of CD34+ cells or hematopoietic stem-progenitor cells, which may be indicative of a disease or condition.

Compositions and kits comprising one or more agents for detecting one or more miRNAs are also provided. An exemplary composition may comprise 1 or more agents, such as probes, that detect, e.g., specifically detect 1, 2, 3, 5, 10, 15, 20, 25, 30, 31, 32 or 33 miRNAs selected from the group consisting of SEQ ID NOs: 1-33 (Table 1) or one or more agents that detect 1, 2, 3, 4, 5, 6, 7, 8 or 9 stem cell miRNAs (Table 6).

Detection may be performed by contacting the sample with a probe or biochip described herein and detecting the amount of hybridization. PCR may be used to amplify nucleic acids in the sample, which may provide higher sensitivity.

The level of the nucleic acid in the sample may also be compared to a control cell (e.g., a normal cell) to determine whether the nucleic acid is differentially expressed (e.g., overexpressed or underexpressed). The ability to identify miRNAs that are differentially expressed in pathological or differentiated cells compared to a control can provide high-resolution, high-sensitivity datasets which may be used in the areas of diagnostics, prognostics, therapeutics, drug development, pharmacogenetics, biosensor development, and other related areas. An expression profile generated by the current methods may be a “fingerprint” of the state of the sample with respect to a number of miRNAs. While two states may have any particular miRNA similarly expressed, the evaluation of a number of miRNAs simultaneously allows the generation of a gene expression profile that is characteristic of the state of the cell. That is, normal tissue may be distinguished from diseased tissue. By comparing expression profiles of tissue in known different disease states, information regarding which miRNAs are associated in each of these states may be obtained. Then, diagnosis may be performed or confirmed to determine whether a tissue sample has the expression profile of normal or disease tissue. This may provide for molecular diagnosis of related conditions.

The expression level of a disease- or differentiation-associated nucleic acid is information in a number of ways. For example, a differential expression of a disease- or differentiation-associated nucleic acid compared to a control may be used as a diagnostic that a patient suffers from the disease. Expression levels of a disease- or differentiation-associated nucleic acid may also be used to monitor the treatment and disease state of a patient. Furthermore, expression levels of a disease- or differentiation-associated miRNA may allow the screening of drug candidates for altering a particular expression profile or suppressing an expression profile associated with disease or state of differentiation.

A target nucleic acid may be detected and levels of the target nucleic acid measured by contacting a sample comprising the target nucleic acid with a biochip comprising an attached probe sufficiently complementary to the target nucleic acid and detecting hybridization to the probe above control levels.

The target nucleic acid may also be detected by immobilizing the nucleic acid to be examined on a solid support such as nylon membranes and hybridizing a labeled probe with the sample. Similarly, the target nucleic may also be detected by immobilizing the labeled probe to a solid support and hybridizing a sample comprising a labeled target nucleic acid. Following washing to remove the non-specific hybridization, the label may be detected.

The target nucleic acid may also be detected in situ by contacting permeabilized cells or tissue samples with a labeled probe to allow hybridization with the target nucleic acid. Following washing to remove the non-specifically bound probe, the label may be detected.

These assays can be direct hybridization assays or can comprise sandwich assays, which include the use of multiple probes, as is generally outlined in U.S. Pat. Nos. 5,681,702; 5,597,909; 5,545,730; 5,594,117; 5,591,584; 5,571,670; 5,580,731; 5,571,670; 5,591,584; 5,624,802; 5,635,352; 5,594,118; 5,359,100; 5,124,246; and 5,681,697, each of which is hereby incorporated by reference.

A variety of hybridization conditions may be used, including high, moderate and low stringency conditions as outlined above. The assays may be performed under stringency conditions which allow hybridization of the probe only to the target. Stringency can be controlled by altering a step parameter that is a thermodynamic variable, including, but not limited to, temperature, formamide concentration, salt concentration, chaotropic salt concentration pH, or organic solvent concentration.

Hybridization reactions may be accomplished in a variety of ways. Components of the reaction may be added simultaneously, or sequentially, in different orders. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors and anti-microbial agents may also be used as appropriate, depending on the sample preparation methods and purity of the target.

### Diagnostic

A method of diagnosis is also provided. The method comprises detecting a differential expression level of a disease- or differentiation-associated nucleic acid in a biological sample. The sample may be derived from a patient. Diagnosis of a disease or state of differentiation in a patient may allow for prognosis and selection of therapeutic strategy.

In one embodiment, a method permits the diagnostic or prognosis of diseases that are characterized by or associated with an abnormal number or location of incompletely differentiated cells, e.g., stem-progenitor cells, e.g. HSPCs. Exemplary diseases include cancer, leukemia, and other diseases described herein. For example, the presence of an unsually or abnormally high number of stem or stem-progenitor cells may be indicative of the presence or likelihood of development of a disease. Monitoring the presence and/or number of stem-progenitor cells may also be used to monitor the progression of a diseas and/or the response to treatment.

In situ hybridization of labeled probes to tissue arrays may be performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes. Such methods may be performed in vitro or in vivo.

In certain embodiments, the miRNAs presented herein are useful to determine the lineage of a cell and to classify a cell or pathological condition. The expression level of these miRNAs can also be correlated with clinical data.

### Screening Assays

The present invention also contemplates the screening of compounds, e.g., peptides, polypeptides, nucleic acids or small molecules, for various abilities to mimic, or interfere with the function of the miRNAs described herein. In the screening assays of the present invention, the candidate substance may first be screened for basic biochemical activity—e.g., binding to a target RNA sequence, inhibition of miRNA binding thereto, alteration in gene expression—and then further tested for function in at the cellular or whole animal level.

The present invention provides methods of screening for agents that alter the activity or expression of miRNAs. As used herein, the term “candidate substance” refers to any molecule that may potentially modulate the function of one or more of the miRNAs in SEQ ID NOS: 1-34. The candidate substance may be a protein, peptide, or a small molecule inhibitor, or even a nucleic acid molecule.

One may acquire, from various commercial sources, small molecule libraries that are believed to meet the basic criteria for useful drugs in an effort to identify useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be a polypeptide, polynucleotide, small molecule or any other compounds that may be developed through random discovery or rational drug design starting from known compounds that affect these miRNAs.

It will, of course, be understood that all the screening methods of the present invention are useful in themselves notwithstanding the fact that effective candidates may not be found. The invention provides methods for screening for such candidates, not solely methods of finding them.

Various cells naturally express one or more of the miRNAs in SEQ ID NOS: 1-34 and can be utilized for screening of candidate substances. In one embodiment, hematopoetic cells are used, or progenitors thereof Other cells may be engineered to express one or more of the miRNAs in SEQ ID NOS: 1-34, or may contain the control regions for these miRNAs linked to screenable marker genes, permitting one to assess the effects of a candidate substance on the expression of one or more of the miRNAs in SEQ ID NOS: 1-34.

Assays may be employed within the scope of the instant invention for determination of the relative efficiency of gene expression. Gene expression may be determined by measuring the production of miRNA in question, or by use of a reporter. The product may be isolated and/or detected by methods well known in the art. Following detection, one may compare the results seen in a given cell line or individual with a reference group of non-transformed control cells.

Significantly, RT-PCR (reverse transcription-polymerase chain reaction) is the most sensitive technique for mRNA quantitation currently available. Compared to the two other commonly used techniques for quantifying mRNA levels, Northern blot analysis and RNase protection assay, RT-PCR can be used to quantify mRNA levels from much smaller samples. In fact, this technique is sensitive enough to enable quantitation of RNA from a single cell.

Over the last several years, the development of novel chemistries and instrumentation platforms enabling detection of PCR products on a real-time basis has led to widespread adoption of real-time RT-PCR as the method of choice for quantitating changes in gene expression. At the start of any PCR reaction, the amplification proceeds at a constant, exponential rate, due to the excess of reagents. The reaction rate ceases to be exponential and enters a linear phase of amplification, after which the amplification rate drops to near zero (plateaus), and little more product is made. In order to accurately assess nucleic acid quantiteis, it is necessary to collect data at a point in which every sample is in the exponential phase of amplification, since it is only in this phase that amplification is extremely reproducible. Unfortunately, the point at which this transition takes place is highly variable. Real-time PCR automates this otherwise laborious process by quantitating reaction products for each sample in every cycle. The result is an amazingly broad 10⁷-fold dynamic range, with no user intervention or replicates required.

Another option for quantitating RNA species is relative quantitative RT-PCR, which uses primers for an internal control that are multiplexed in the same RT-PCR reaction with the gene specific primers. Internal control and gene-specific primers must be compatible, i.e., they must not produce additional bands or hybridize to each other. Common internal controls include .beta.-actin and GAPDH mRNAs and 18S rRNA. Unlike Northerns and nuclease protection assays, the selection and implementation of controls in relative quantitative RT-PCR requires substantial optimization.

Other assays provided herein allow for the identication of genes that are involved in stem cell differentation and proliferation or other characteristic of a stem or stem-progenitor cell. An assay may comprise identifying a gene that comprises, e.g., in an untranslated region, a nucleotide sequence that is signigicantly identical to or complementary to the sequence of a miRNA identified herein as involved in stem-progenitor cell differentiation, e.g., a miRNA set forth in Table 1 or 6.

### Gene Silencing

A method of reducing expression of a target gene in a cell, tissue or organ is also provided. Expression of the target gene may be reduced by expressing a nucleic acid described herein that comprises a sequence substantially complementary to one or more binding sites of the target mRNA. The nucleic acid may be a miRNA comprising SEQ ID NOS: 1-33 or a variant thereof. The nucleic acid may also be pri-miRNA, pre-miRNA, or a variant thereof, which may be processed to yield a miRNA. The expressed miRNA may hybridize to a substantially complementary binding site on the target mRNA. An example for a study employing over-expression of miRNA is Yekta et al 2004, Science 304-594, which is incorporated herein by reference. One of ordinary skill in the art will recognize that the nucleic acids described herein may also be used to inhibit expression of target genes or inhibit activity of miRNAs using antisense methods well known in the art, as well as RNAi methods described in U.S. Pat. Nos. 6,506,559 and 6,573,099, which are incorporated by reference.

The target of gene silencing may be a protein that causes the silencing of a second protein. By repressing expression of the target gene, expression of the second protein may be increased. Examples for efficient suppression of miRNA expression are the studies by Esau et al 2004 JBC 275-52361; and Cheng et al 2005 Nucleic Acids Res. 33-1290, which is incorporated herein by reference. Such methods may be performed in vitro or in vivo.

### Enhancement of Gene Expression

A method of increasing expression of a target gene in a cell, tissue or organ is also provided. Expression of the target gene may be increased by expressing a nucleic acid described herein that comprises a sequence substantially complementary to a pri-miRNA, pre-miRNA, miRNA or a variant thereof, e.g., a complement of SEQ ID NOS: 1-34. The nucleic acid may be an anti-miRNA. The anti-miRNA may hybridize with a pri-miRNA, pre-miRNA or miRNA, thereby reducing its gene repression activity. Expression of the target gene may also be increased by expressing a nucleic acid that is substantially complementary to a portion of the binding site in the target gene, such that binding of the nucleic acid to the binding site may prevent miRNA binding. Such methods may be performed in vitro or in vivo.

### Therapeutics

Modulating the differentiation of stem cells, e.g., hematopoietic stem cells, may be used for treating or preventing a disease or condition. For example, a subject may receive an agonist of one or more of the miRNAs having any one of SEQ ID NOs: 1-33 (“HSPC miRNAs) or of one of the stem cell miRNAs to block cell differentiation in situations in which an excessive cell differentiation is present and/or insufficient amounts of undifferentiated cells are present in the subject. Such conditions may include those in which a subject has been treated, e.g., by irradiation and has lost a considerable amount of undifferentiated cells or HSPCs. A subject may receive one or more agonists to thereby increase the number of stem progenitor cells.

In other embodiments, in which it is necessary to increase the number of differentiated cells in a subject, a treatment may comprise administering an agonist of one or more stem cell miRNA or HSPC miRNA. For example, a subject having cancer may be treated by receiving one or more antagonists of one or stem cell miRNA or HSPC miRNA, such as to stimulate the differentiation of an undifferentiated cancer cell to thereby reduce the number of cancer cells. Other conditions in which one may want to induce the differentiation of a stem-progenitor cell include those associated with an insufficient number of a differentiated cell, e.g., a differentiated or partially differentiated hematopoietic cell. Exemplary conditions or diseases that may be treated with antagonists include those associated with insufficient leukocytes (leukopenia, e.g., graynulocytosis, neutropenia such as drug or chemotherapy induced neutropenia, and congenital neutropenia); insufficient lymphocytes, insufficient red blood cells (e.g., anemias, such as aplastic anemia, nutritional deficiency anemia, hemolytic anemia); or insufficient platelets (thrombocytopenia). Other diseases include immunodeficiency diseases, e.g., inherited immunodeficiency diseases, drug or therapy induced immunodeficiency diseases, HIV-immunodeficiency diseases. Yet other diseases in which differentiation of cells may be beneficial include blood cancers in which undifferentiated or partially differentiated cells accumulate, e.g., leukemias and lymphomas (e.g., Hodgkin's and non Hodgkin's lymphomas, acute leukemias, e.g., lymphoblastic (ALL) and myelogenous (AML) and chronic leukemias).

Other conditions that may benefit from methods of modulating stem-progenitor cell differentiation include stem cell transplantation, graft versus host disease, and transplantation, e.g., bone marrow transplantation.

Methods described herein may also be used for stem cell treatments. Diseases that would benefit from stem cell treatments include Parkinson's disease, spinal cord injuries (as well as other neuromuscular or neurological degenerative diseases), cancer, muscle damage, bone marrow transplants, leukemias and lymphomas. Generally, disorders that can be treated by infusion of stem cells include but are not limited to five broad categories. First are diseases resulting from a failure or dysfunction of normal blood cell production and maturation (i.e., aplastic anemia and hypoproliferative stem cell disorders). The second group are neoplastic, malignant diseases in the hematopoietic organs (e.g., leukemias, lymphomas, myelomas):The third group of disorders comprises those of patients with a broad spectrum of malignant solid tumors of non-hematopoietic origin. Stem cell infusion in these patients serves as a bone marrow rescue procedure, which is provided to a patient following otherwise lethal chemotherapy or irradiation of the patient, designed to eliminate malignant tumor cells. The fourth group of diseases consists of autoimmune conditions, where the stem cells serve as a source of replacement of an abnormal immune system. The fifth group of diseases comprises a number of genetic disorders which can be corrected by infusion of hematopoietic stem cells, preferably syngeneic, which prior to transplantation have undergone gene therapy. Particular diseases and disorders which can be treated by hematopoietic reconstitution with substantially enriched population of hematopoietic stem cells include but are not limited to those listed here: Diseases resulting from a failure or dysfunction of normal blood (cell production and maturation, hyperproliferative stem cell disorders, aplastic anemia, pancytopenia, agranulocytosis, thrombocytopenia, red cell aplasia, Blackfan-Diamond syndrome, due to drugs, radiation, or infection, idiopathic); Hematopoietic malignancies (acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkins's lymphoma); Malignant, solid tumors (malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing's sarcoma, lymphoma); Autoimmune diseases (rheumatoid arthritis, diabetes type I, chronic hepatitis, multiple sclerosis, systemic lupus erythematosus); Genetic (congenital) disorders (anemias, familial aplastic, Fanconi's syndrome, Bloom's syndrome, pure red cell aplasia (PRCA), dyskeratosis congenita, Blackfan-Diamond syndrome, congenital dyserythropoietic syndromes I-IV, Schwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase) variants 1, 2, 3, pyruvate kinase deficiency, congenital erythropoietin sensitivity deficiency, sickle cell disease and trait, thalassemia alpha, beta, gamma, met-hemoglobinemia, congenital disorders of immunity, severe combined immunodeficiency disease (SCID), bare lymphocyte syndrome, ionophore-responsive combined immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes) and Others (osteopetrosis, myelosclerosis, acquired hemolytic anemias, acquired immunodeficiencies, infectious disorders causing primary or secondary, immunodeficiencies, bacterial infections (e.g., Brucellosis, Listerosis, tuberculosis, leprosy), parasitic infections (e.g., malaria, Leishmaniasis), fungal infections, disorders involving disproportions in lymphoid cell sets and, impaired immune functions due to aging, phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, alpha 1-antitrypsin deficiency).

Any of the disorders set forth in the previous paragraph may also be diagnosed as further described herein.

Compositions, e.g., pharmaceutical compositions are also provided herein. A composition may comprise 1, 2, 3, 4, 5, 6, 7, 8, 9,10, 15, 20, 25, 30, 31, 32, 33 or 34 agonists or antagonists or combinations thereof of miRNAs described herein.

In some embodiments, therapeutic compositions and methods may include the use of modified miRNAs or miRNA antagonists, including 2-O-methyl oligoribonucleotides (2-O-Me-RNAs). Cell permeable forms of these 2-O-Me-RNAs, known as antagomirs, are also contemplated. Antagomirs have been successfully used to down-regulate mi-RNAs in mouse tissue, in vivo, after intravenous injection (Krutzfeldt et al., 2005, Nature, 438: 685-689). Other modifications may include the introduction of phosphorothioate linkages, the addition of 2-O-methoxyethyl groups, and the addition of a cholesteryl or cholesterol moiety. Locked nucleic acid molecules, wherein the 2′-O-oxygen of a 2′-O-modified RNA is bridged to the 4′ position via a methylene linker, to form a rigid bicycle locked into a C3′-endo (RNA) sugar conformation are also contemplated.

The following publications are incorporated by reference in their entirety: Mack, 2007, Nature, 25: 631-638; Blenkiron, 2007, Human Molecular Genetics, 16: R106-R113; Hammond, 2006, Trends in Molecular Medicine, 12: 99-101; Krutzfeldt, 2006, Nature Genetics Supplment, 38: S14-S19; Wurdinger and Costa, 2007, The Pharmacogenomics Journal, 7: 297-304; Hunziker and Hall, 2006, Gene Therapy, 13: 496-502.

### Compositions

A pharmaceutical composition is also provided. The composition may comprise a nucleic acid described herein and optionally a pharmaceutically acceptable carrier and/or excipient. The compositions may be used for diagnostic or therapeutic applications. The pharmaceutical composition may be administered by known methods, including wherein a nucleic acid is introduced into a desired target cell in vitro or in vivo. Commonly used gene transfer techniques include calcium phosphate, DEAE-dextran, electroporation, microinjection, viral methods and cationic liposomes. In some embodiments, the compositions may include the use of synthetic miRNAs, which are more potent than naturally occurring miRNAs (Chang D Z, Clin Cancer Res, 2006, 12: 6557-6564).

### Kits

A kit is also provided comprising a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

### Method of Synthesis

A method of synthesizing the reverse-complement of a target nucleic acid is also provided. The reverse complement may be synthesized according to methods outlined in U.S. Pat. No. 11/384,049, the contents of which are incorporated herein by reference.

### Method of Detection

A method of detecting a target nucleic acid in a biological sample is also provided. The target nucleic acid may be detected according to methods outlined in U.S. Pat. No. 11/384,049, the contents of which are incorporated herein by reference.

### Examples

Abbreviations: BM, bone marrow; HE-miRNA, hematopoietic-expressed microRNA; HSPCs, hematopoietic stem-progenitor cells; miRNAs, microRNAs; PBSC, peripheral blood stem cell; RFU, relative fluorescence units.

### Example 1

### Abstract

MicroRNAs (miRNAs) are a recently identified class of epigenetic elements consisting of small noncoding RNAs that bind to the 3′ untranslated region of mRNAs and down-regulate their translation to protein. miRNAs play critical roles in many different cellular processes including metabolism, differentiation, and development. The present inventors have found 33 miRNAs expressed in CD34+ hematopoietic stem-progenitor cells (HSPCs) from normal human bone marrow and mobilized human peripheral blood stem cell harvests. The inventors then combined these data with human HSPC mRNA expression data and with miRNA-mRNA target predictions, into a previously undescribed miRNA:mRNA interaction database called the Transcriptome Interaction Database. The in silico predictions from the Transcriptome Interaction Database pointed to miRNA control of hematopoietic differentiation through translational control of mRNAs critical to hematopoiesis. From these predictions, the inventors formulated a model for miRNA control of stages of hematopoiesis in which many of the genes specifying hematopoietic differentiation are expressed by HSPCs, but are held in check by miRNAs until differentiation occurs. The inventors validated miRNA control of several of these target mRNAs by demonstrating that their translation in fact is decreased by miRNAs. Finally, the inventors chose miRNA-155 for functional characterization in hematopoiesis, because it was predicted that it would control both myelopoiesis and erythropoiesis. As predicted, miRNA-155 transduction greatly reduced both myeloid and erythroid colony formation of normal human HSPCs.

miRNA Expression in CD34+ Cells.

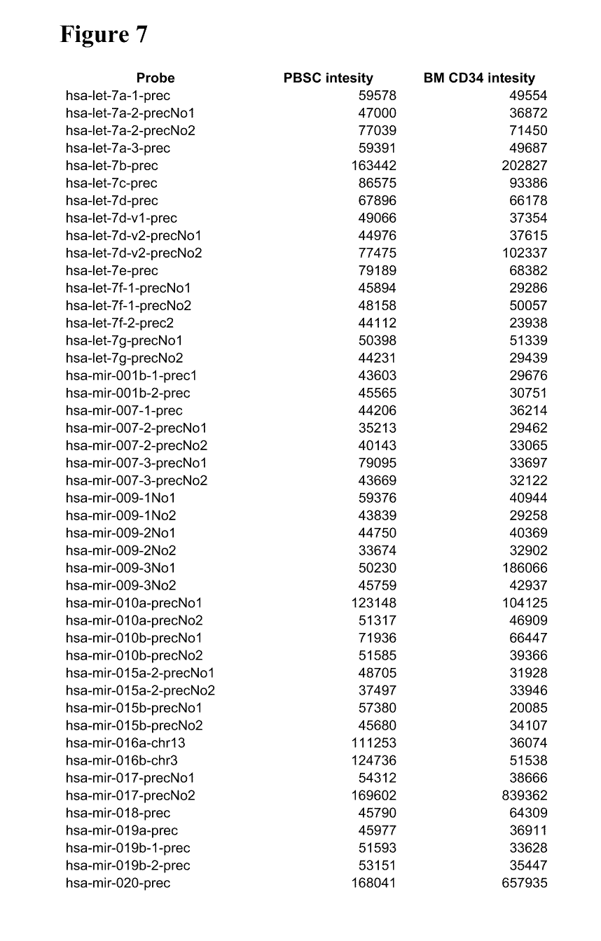

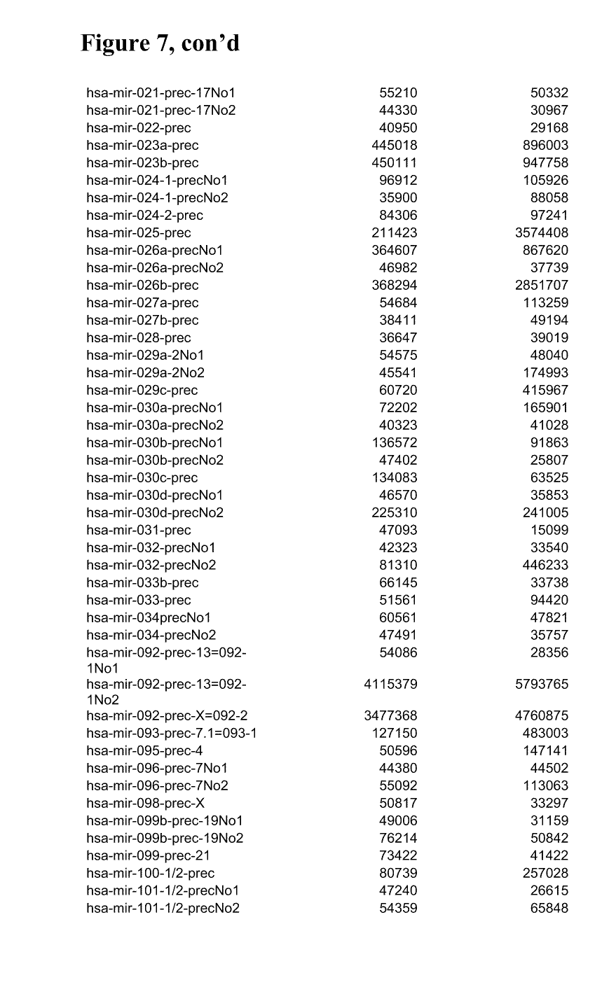

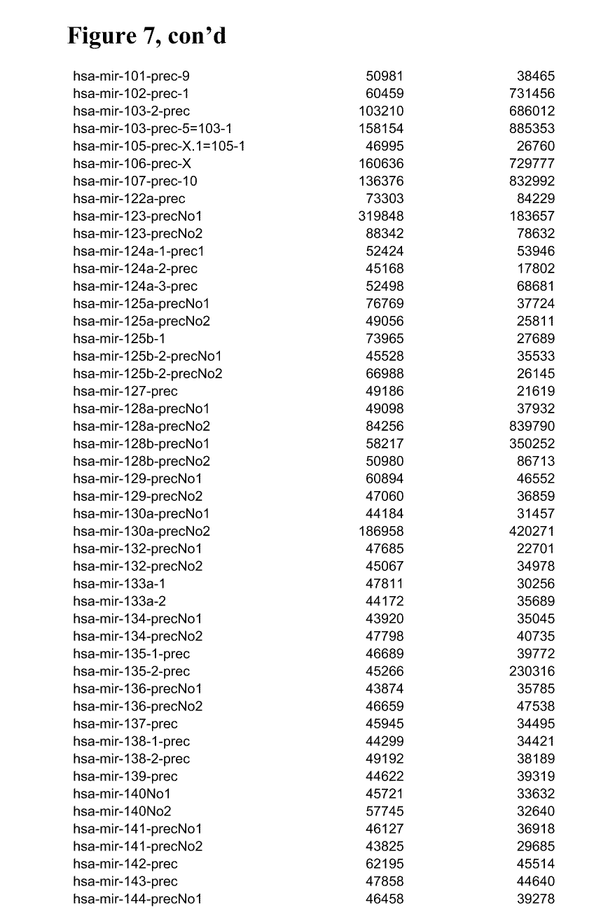

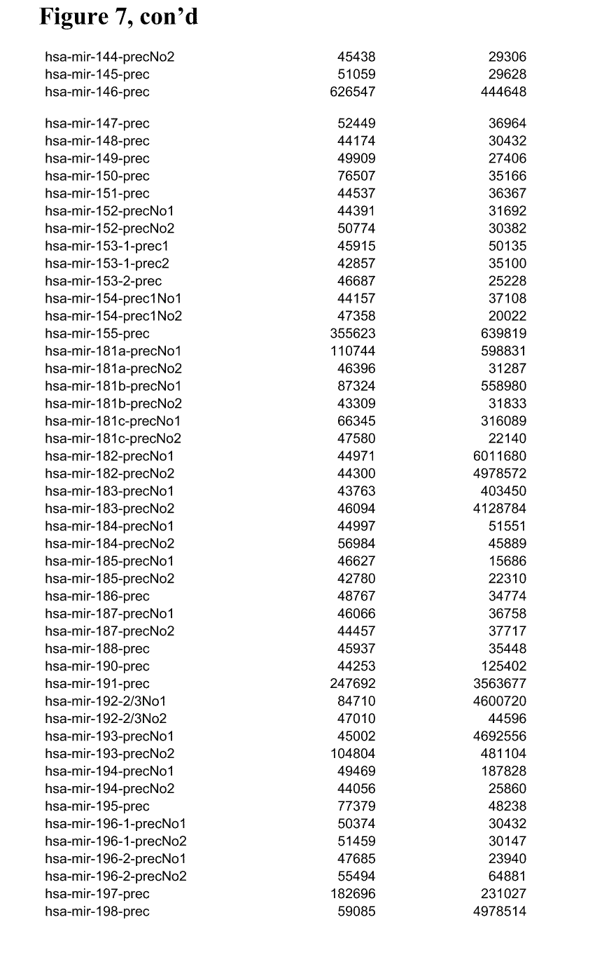

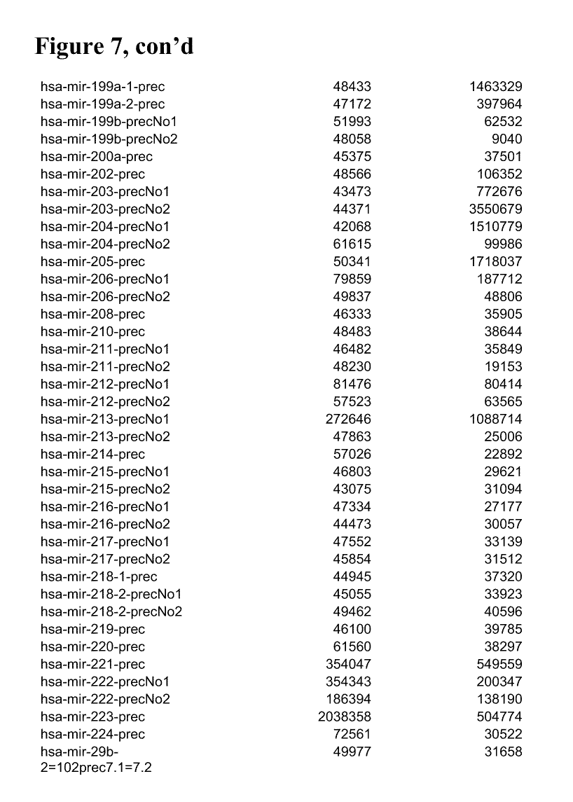

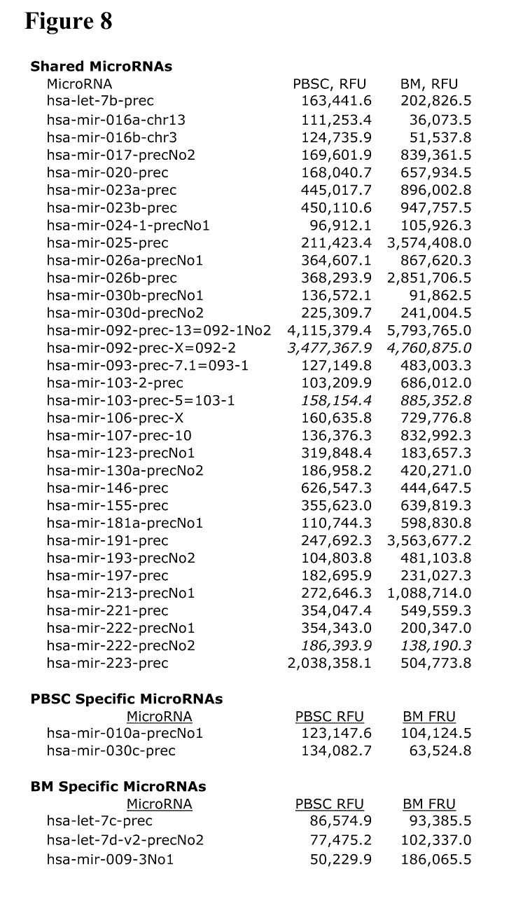

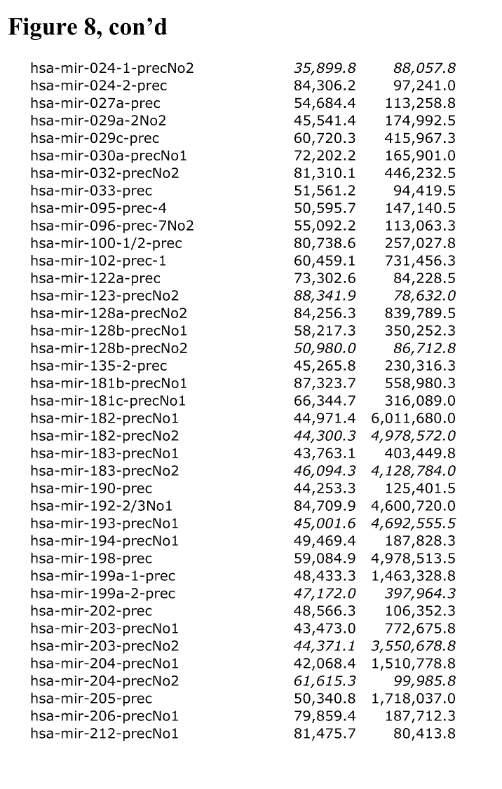

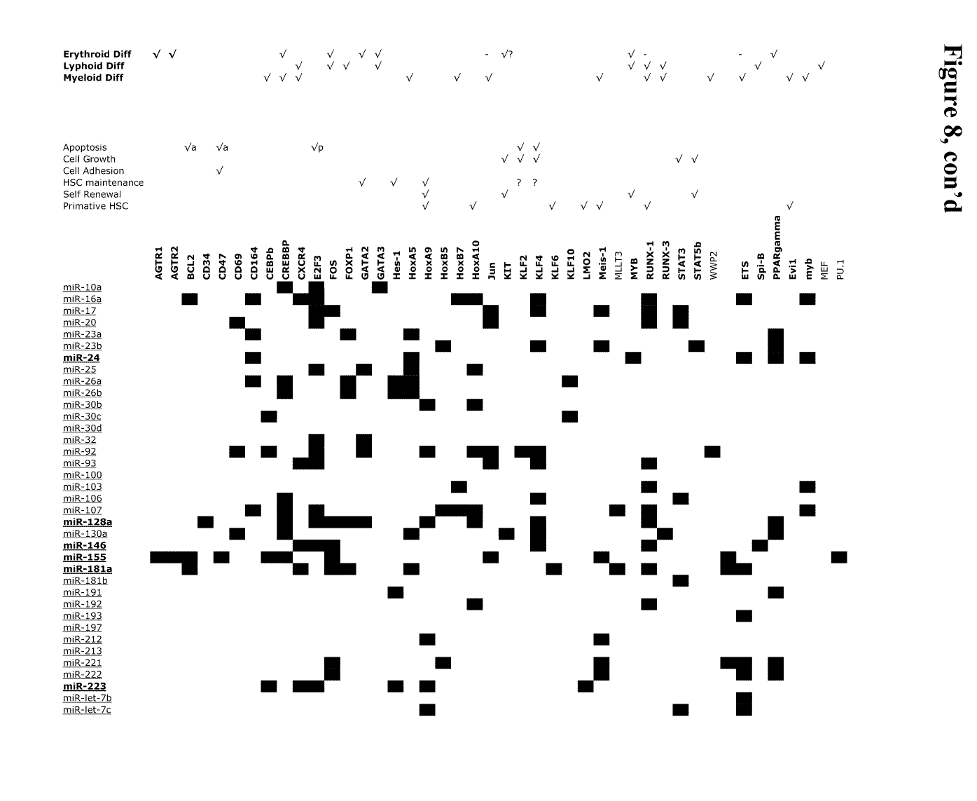

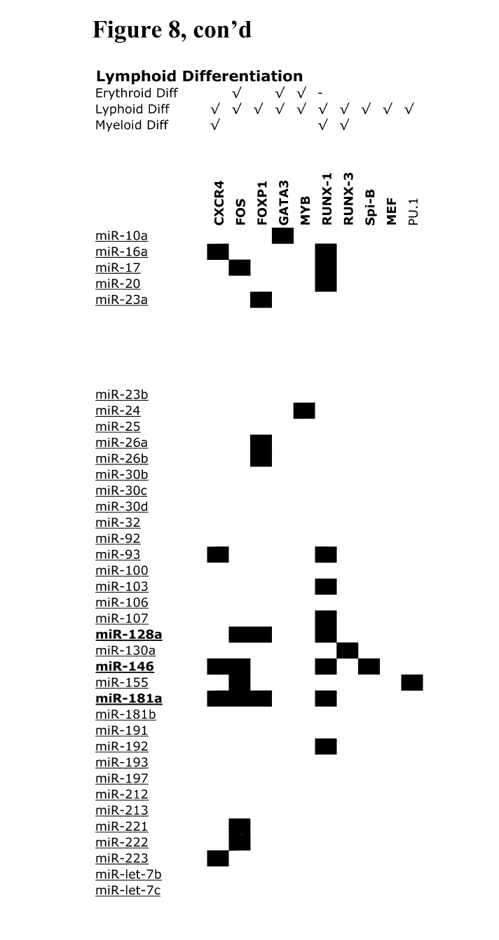

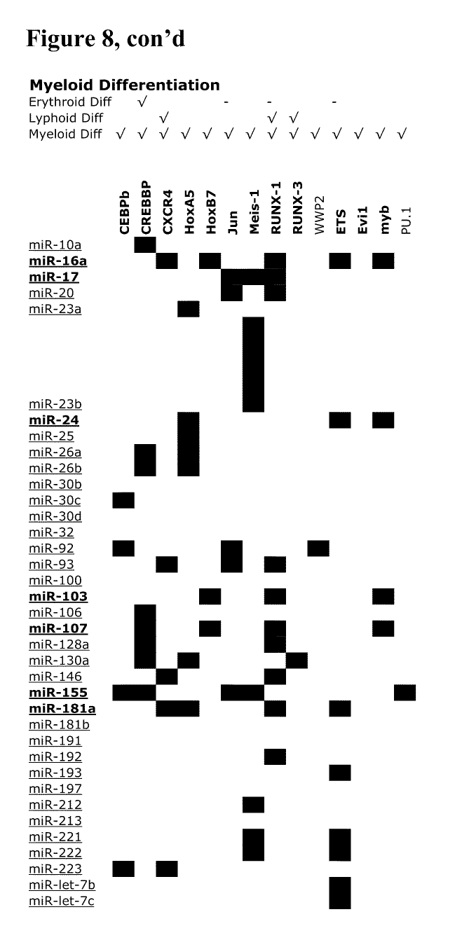

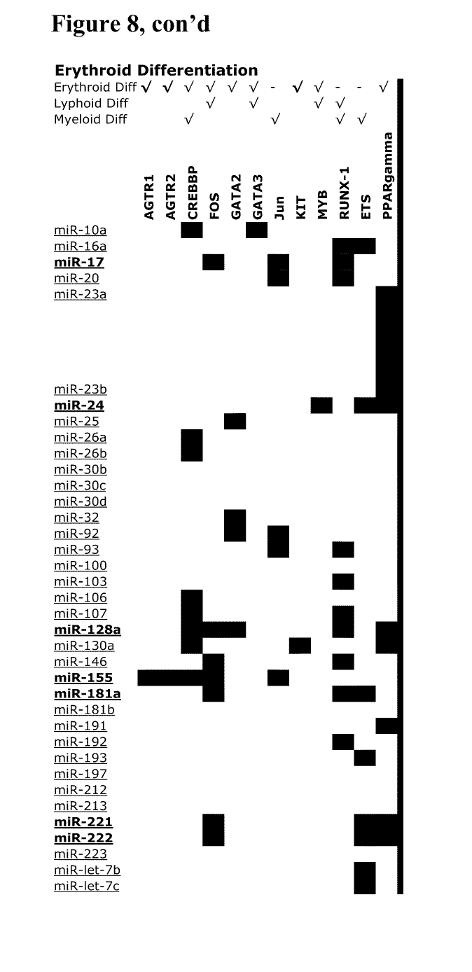

The expression of 228 human miRNAs in CD34+ HSPCs obtained from normal donor PBSC or BM harvests (FIG. 7) was measured. Mean background was 47,458 relative fluorescence units (RFU) for PBSC and 34,676 RFU for BM CD34+ cells. After normalization of the data with GeneSpring software, the minimum cutoff for an expressed miRNA was calculated to be 96,900 RFU for PBSC and 74,509 RFU for BM CD34+ cells. Filtering for expression, in combination with a Student's t test significance threshold of 0.05, yielded 35 miRNAs expressed in PBSC and 78 miRNAs in BM CD34+ cells (FIG. 8). Intersection analysis (8) of these data sets (Table 1) identified 31 mature miRNAs and 2 precursor miRNAs, 33 miRNAs in total, expressed by CD34+ cells from both PBSC and BM, which were designated hematopoietic-expressed-miRNAs.

HE-miRNAs are Predicted to Regulate Several mRNAs Expressed in CD34+ Cells.

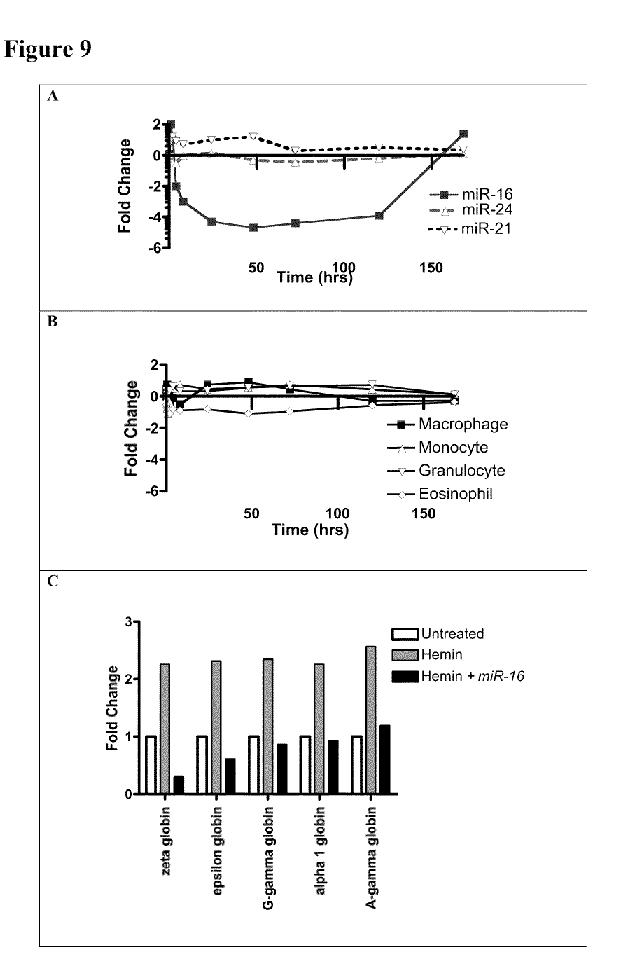

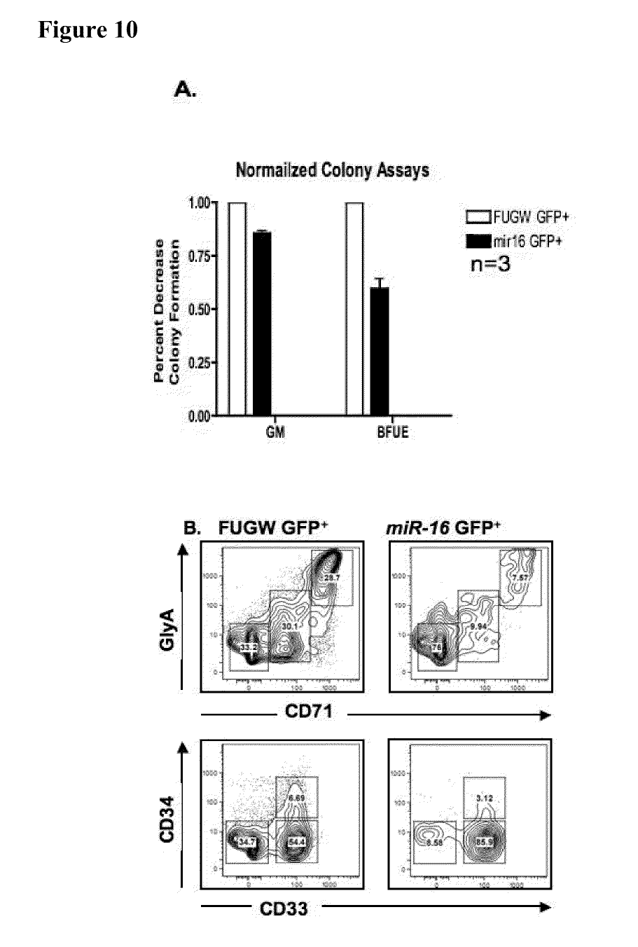

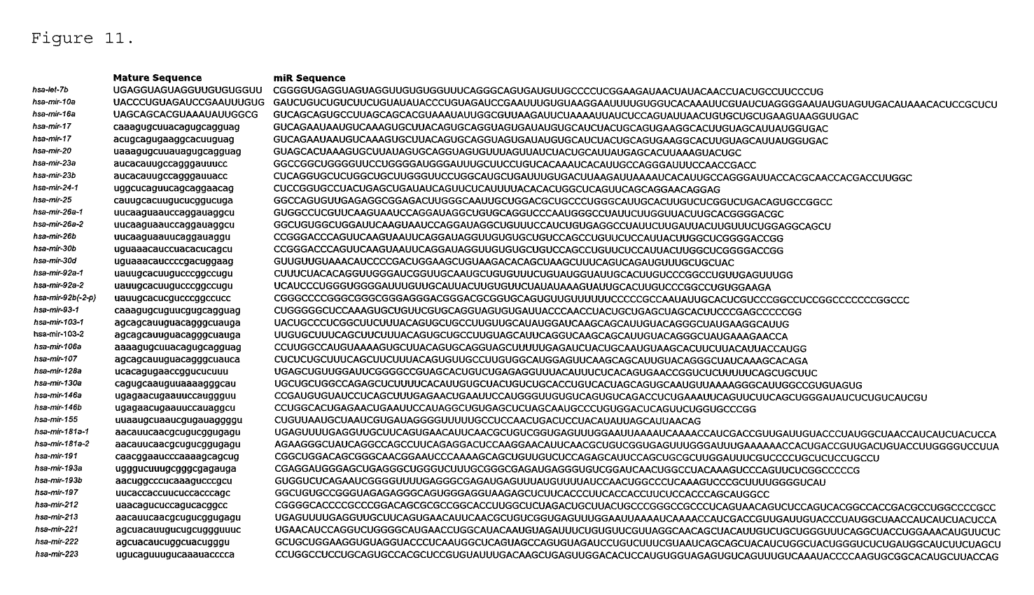

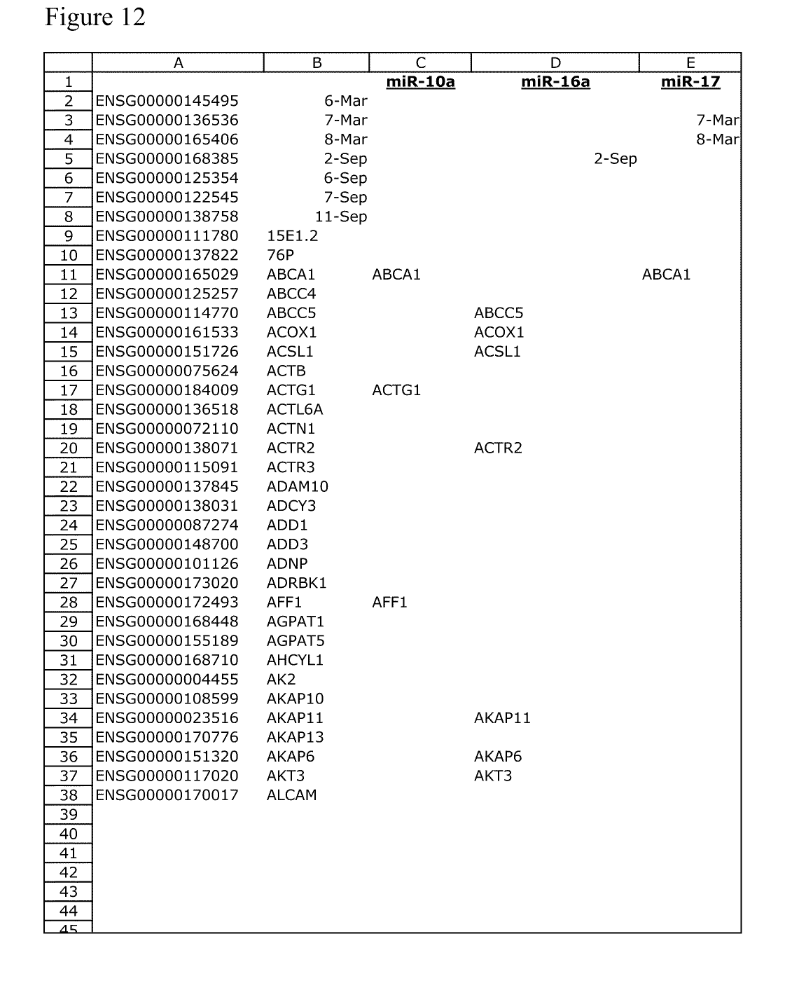

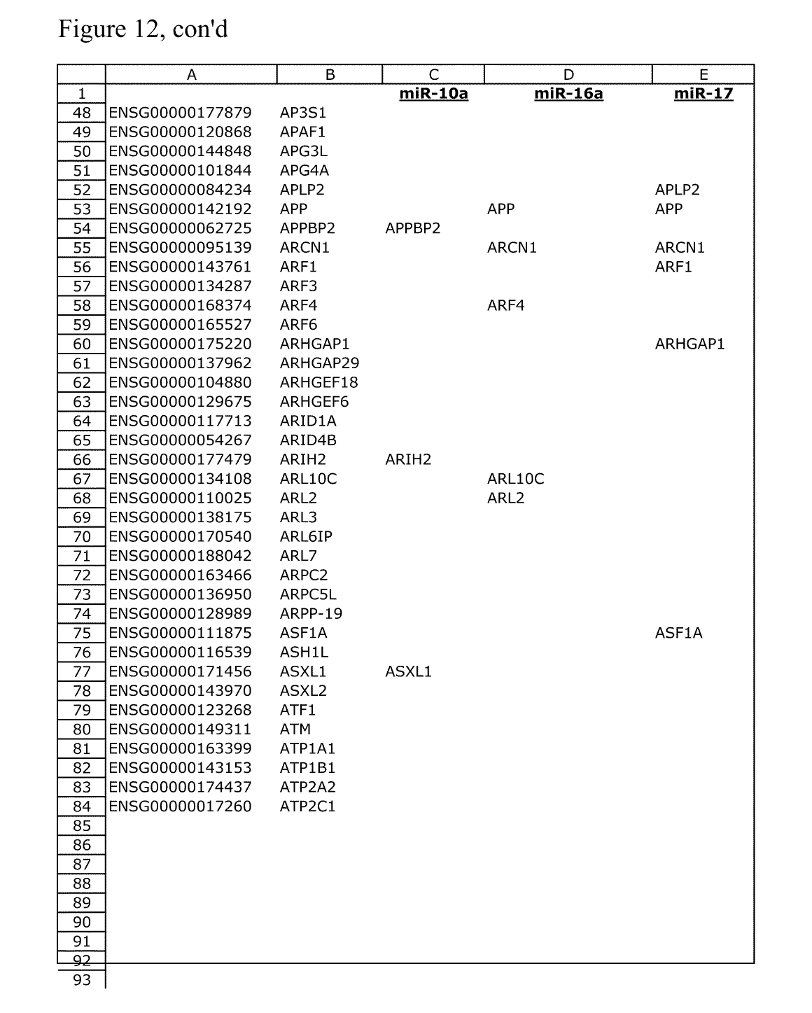

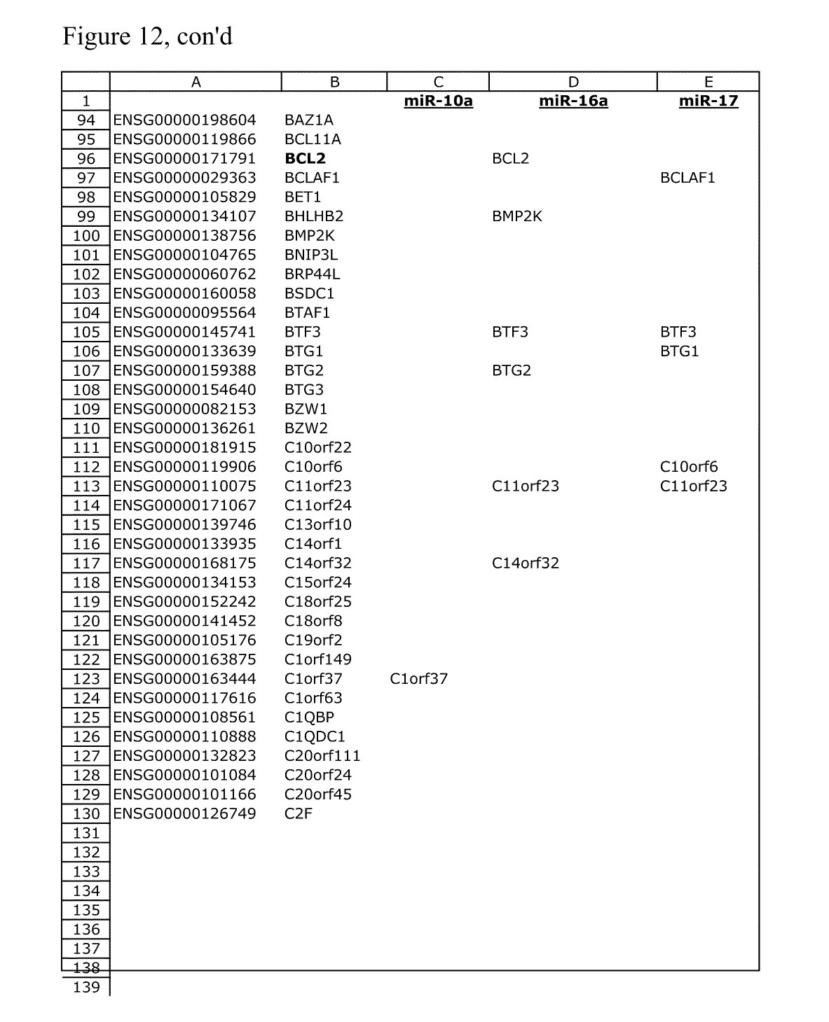

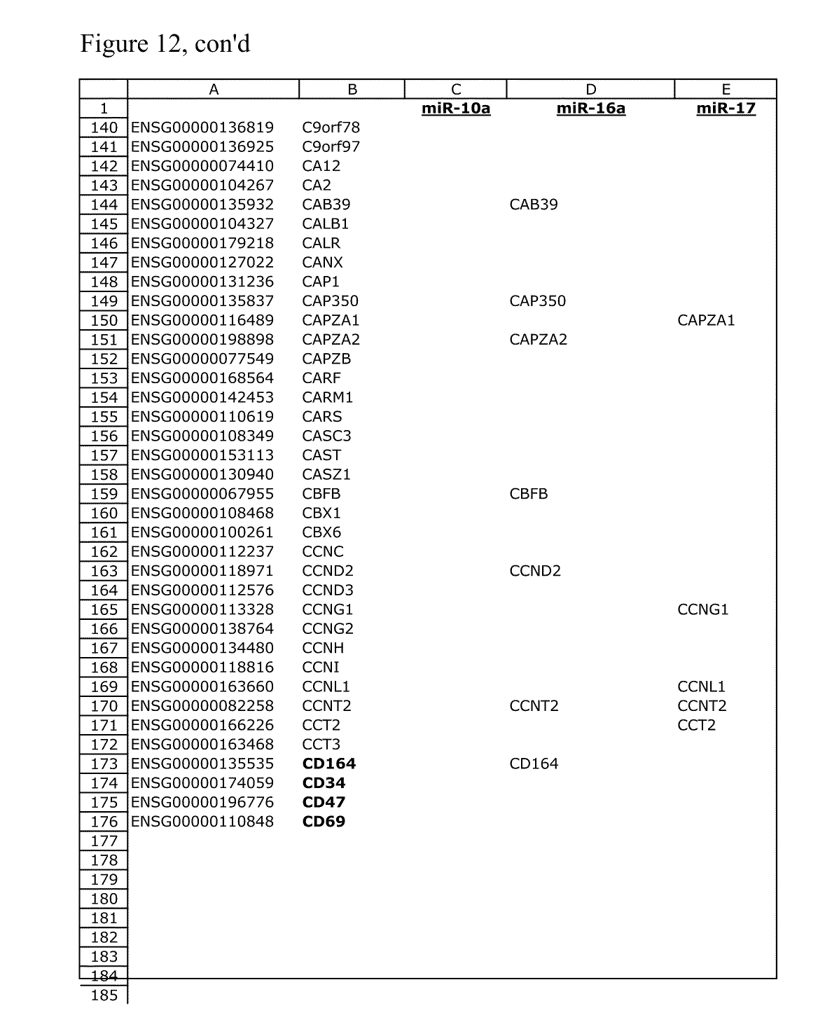

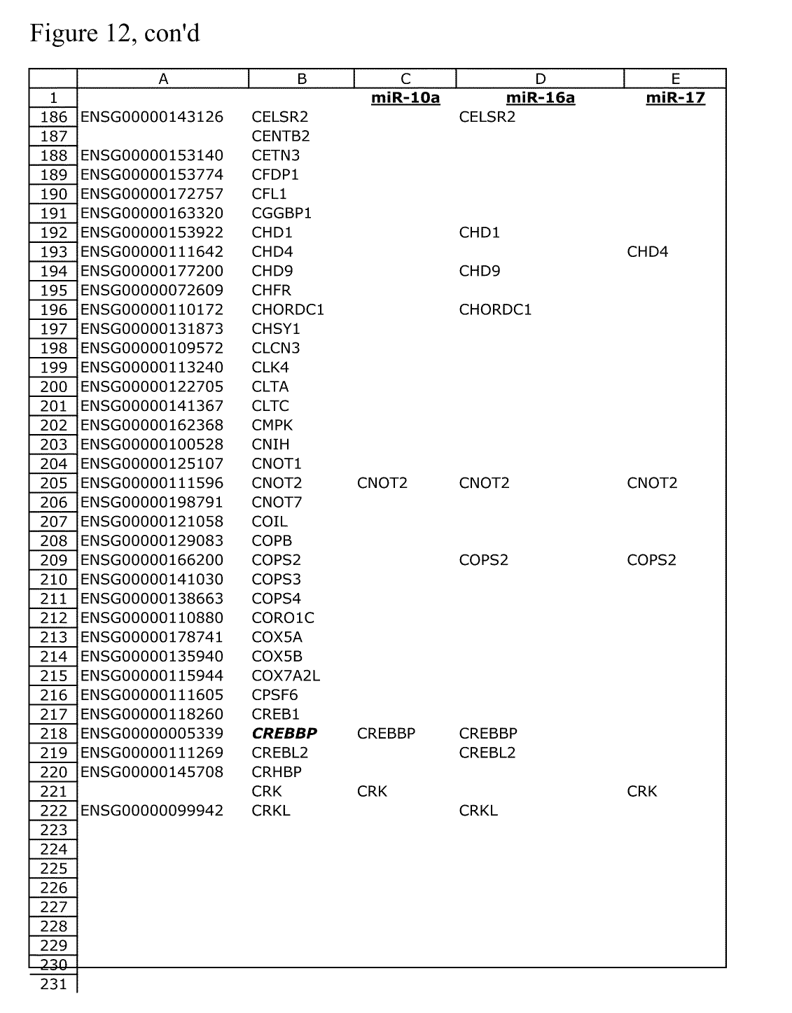

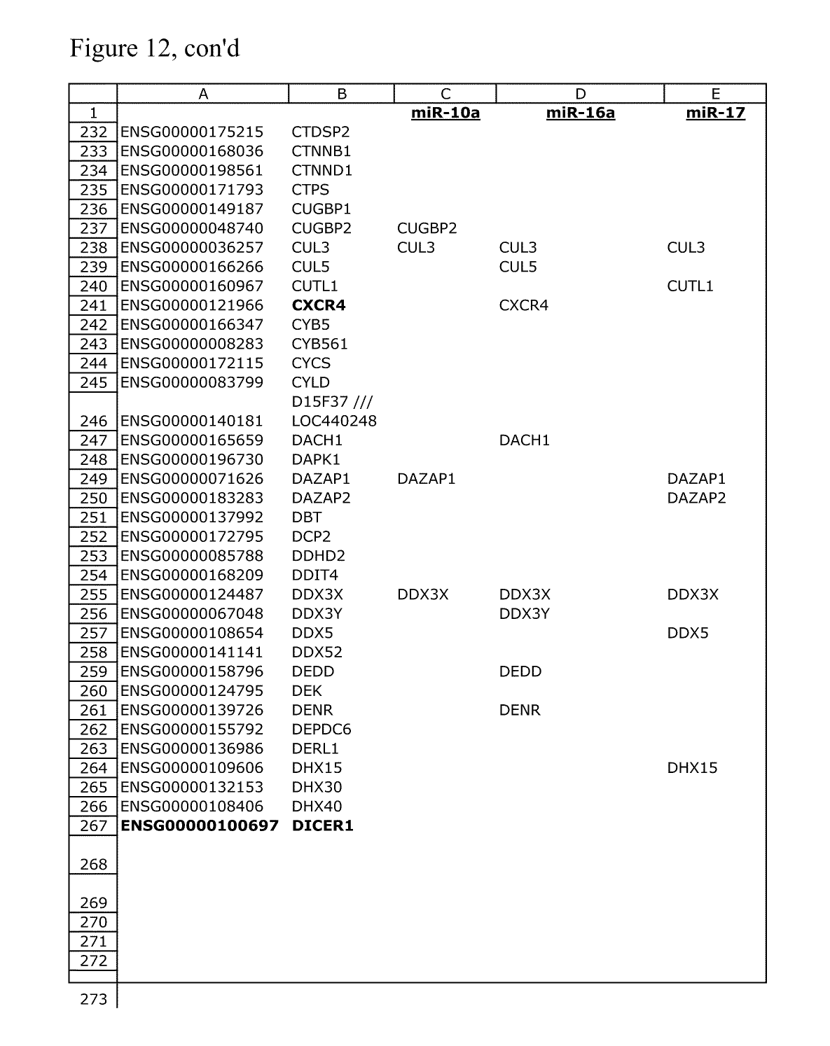

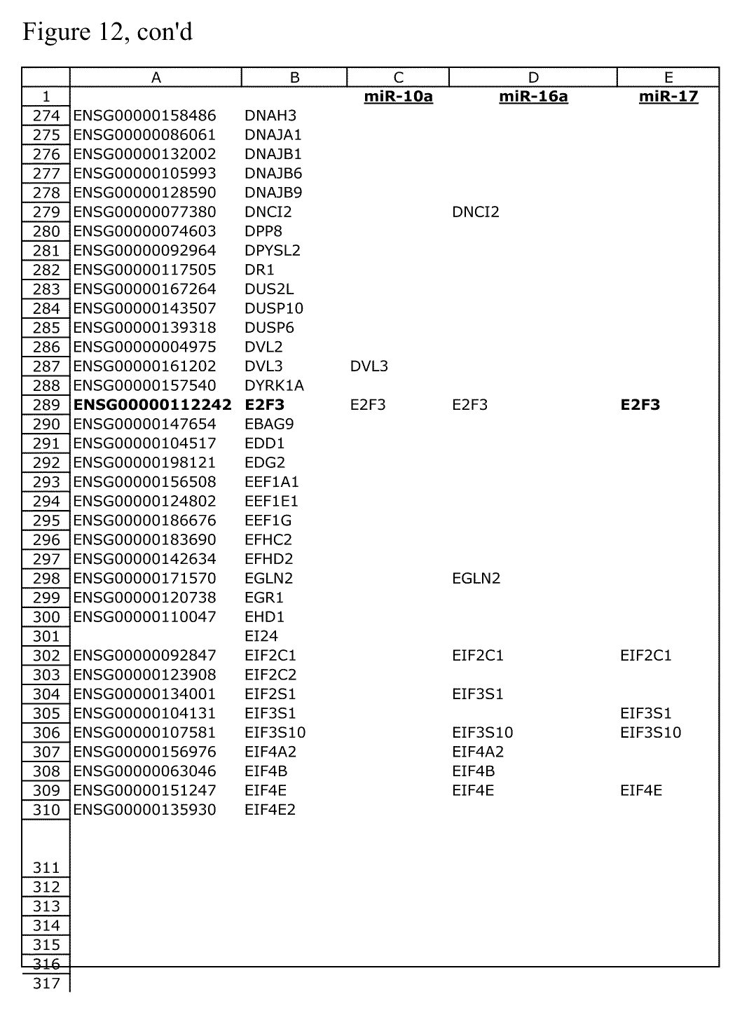

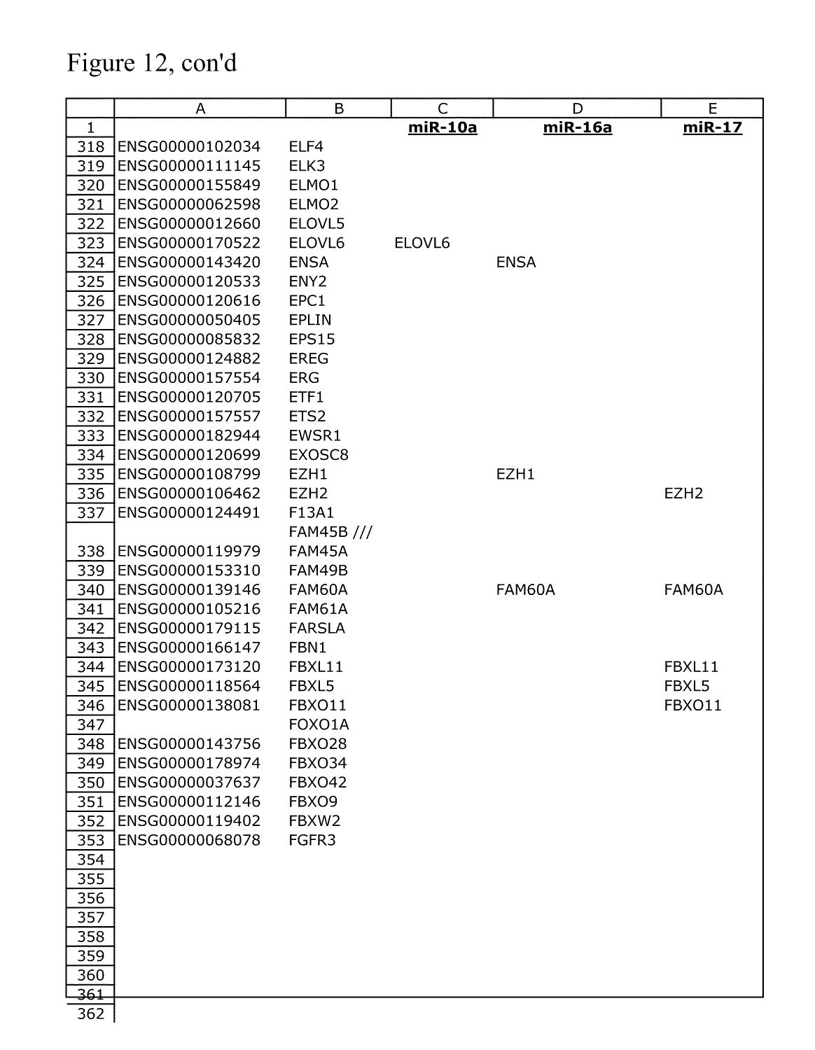

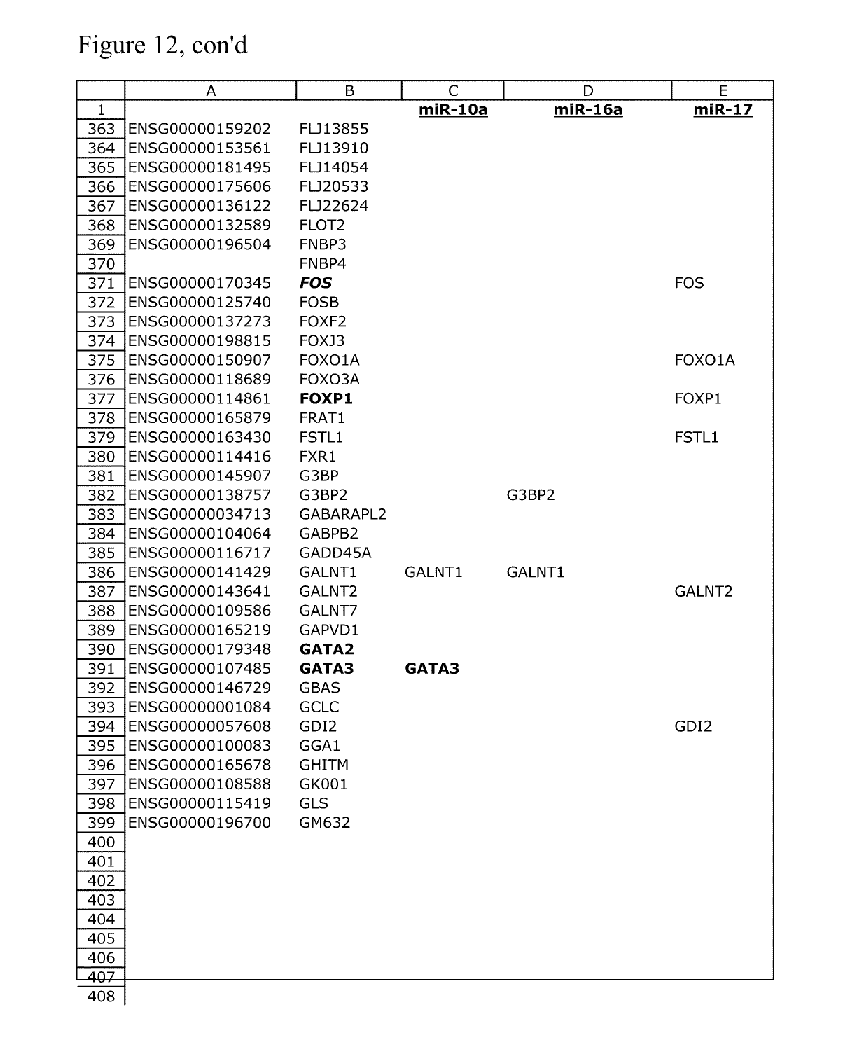

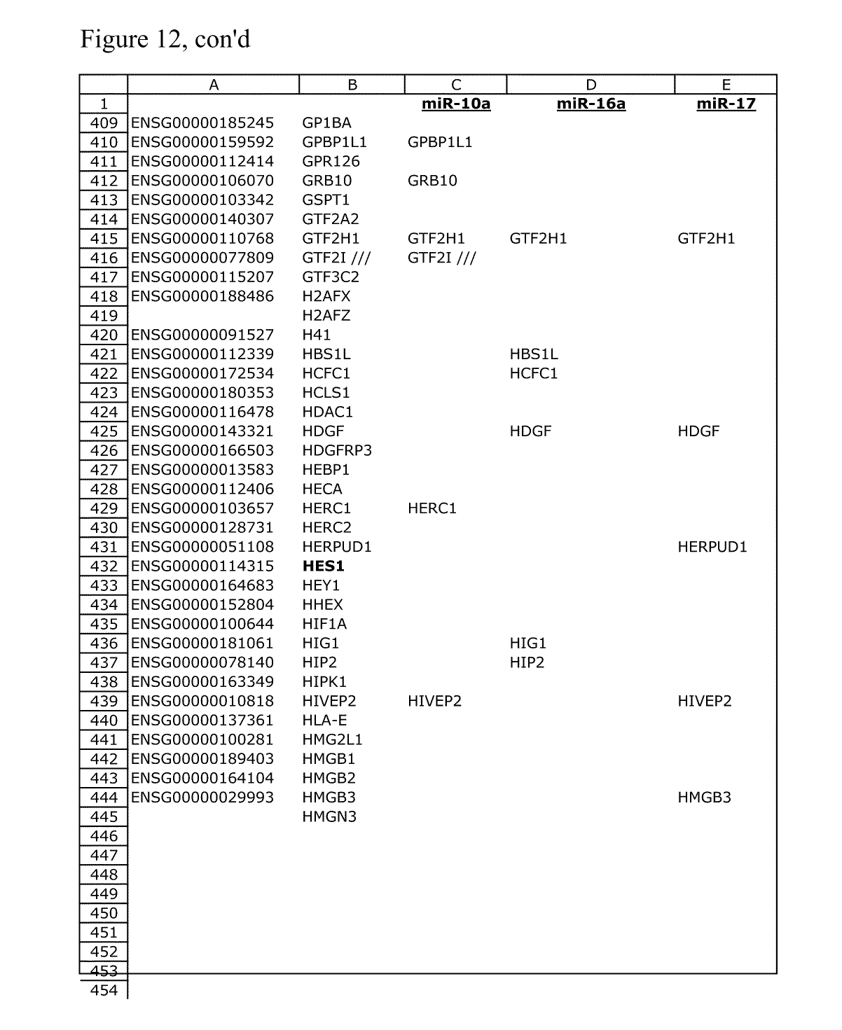

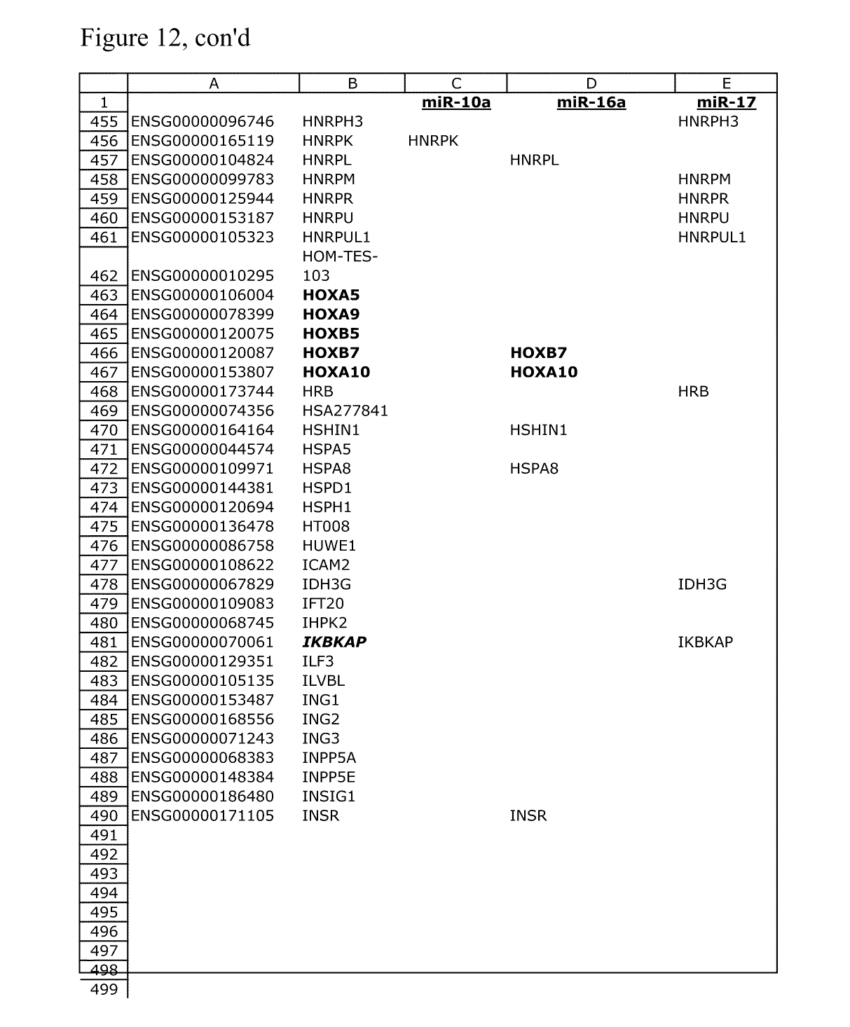

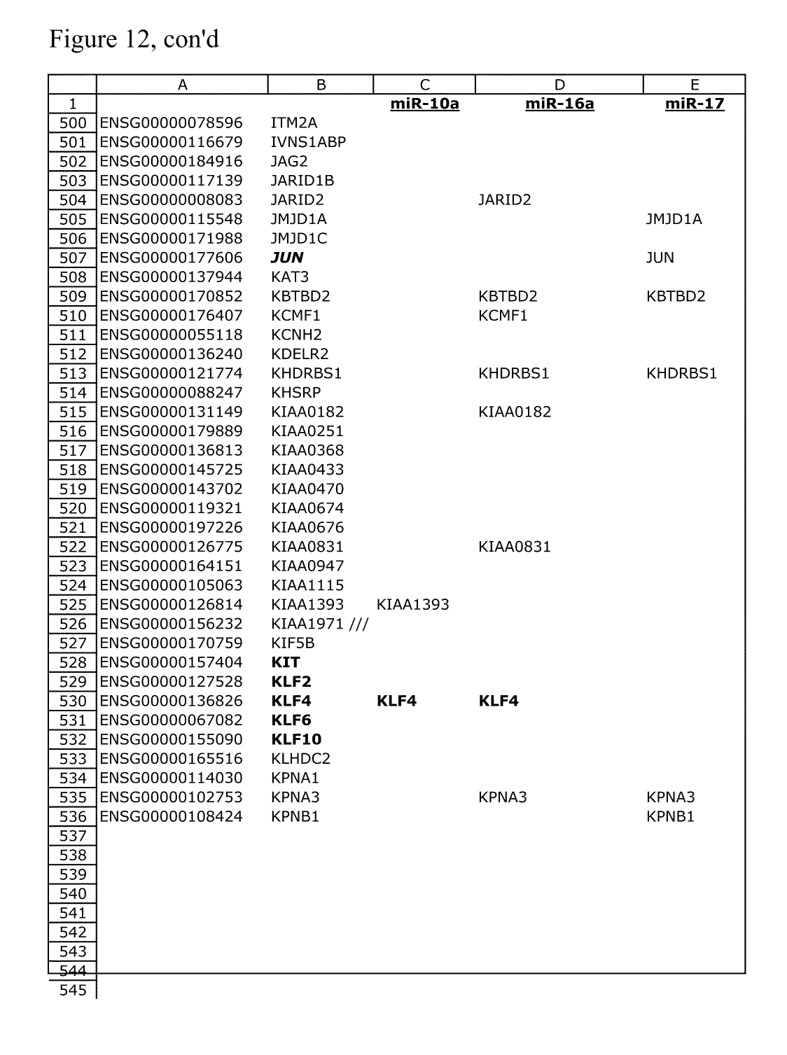

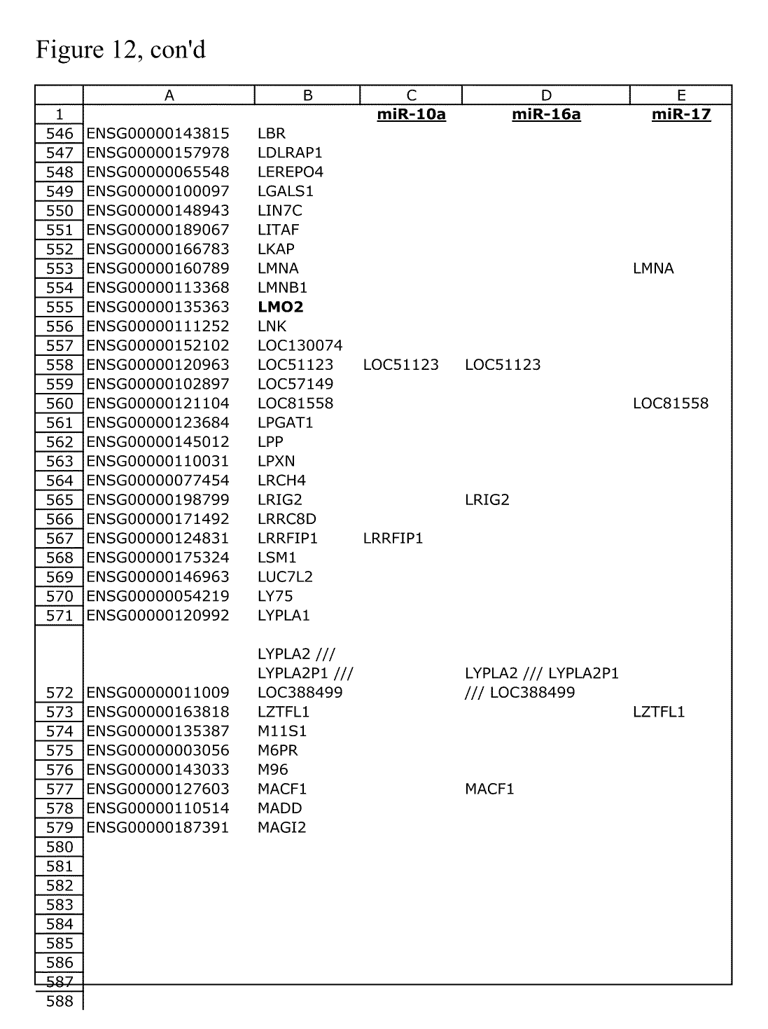

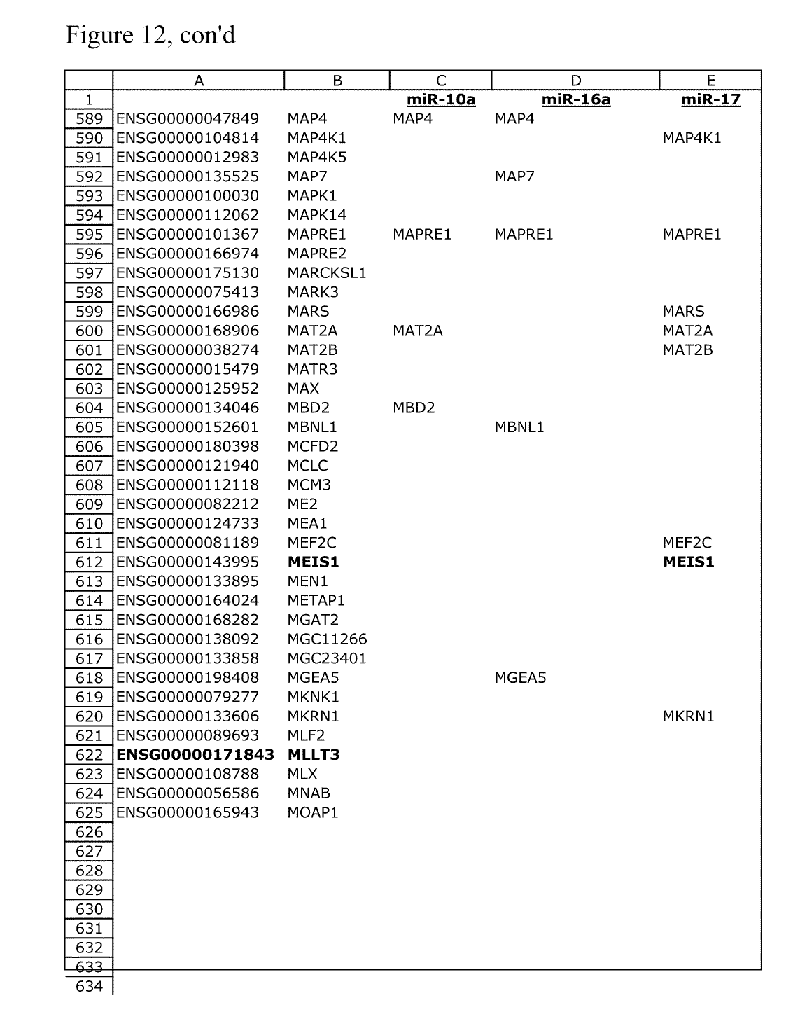

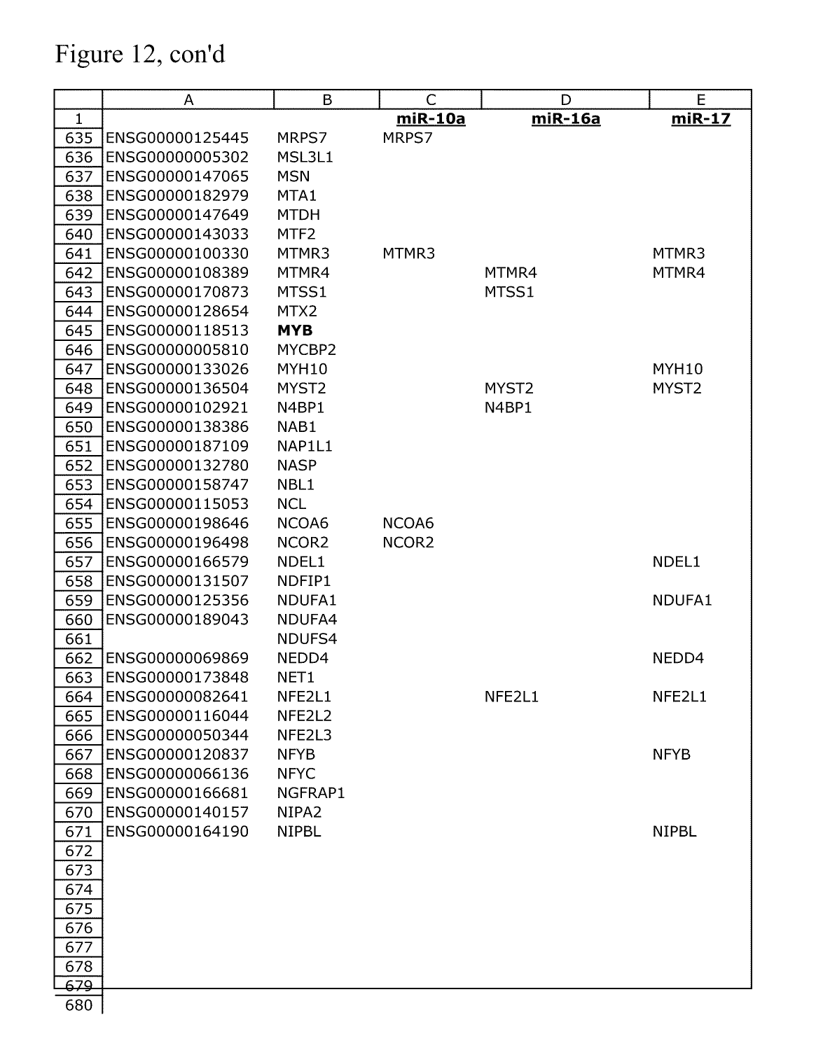

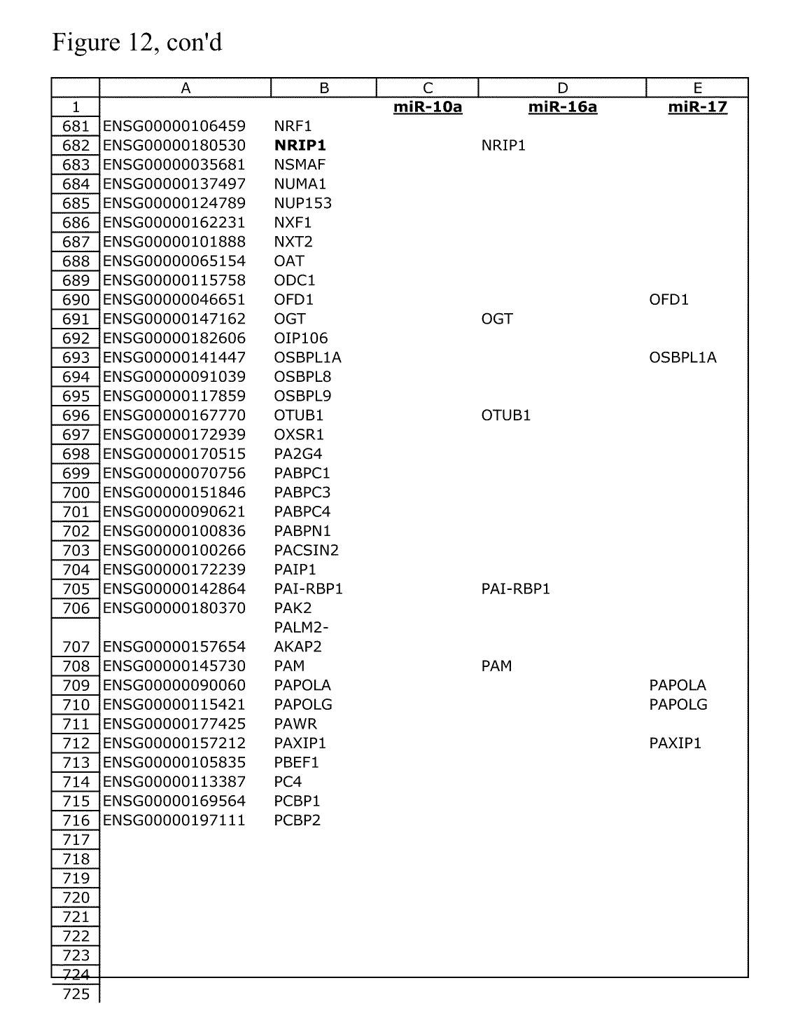

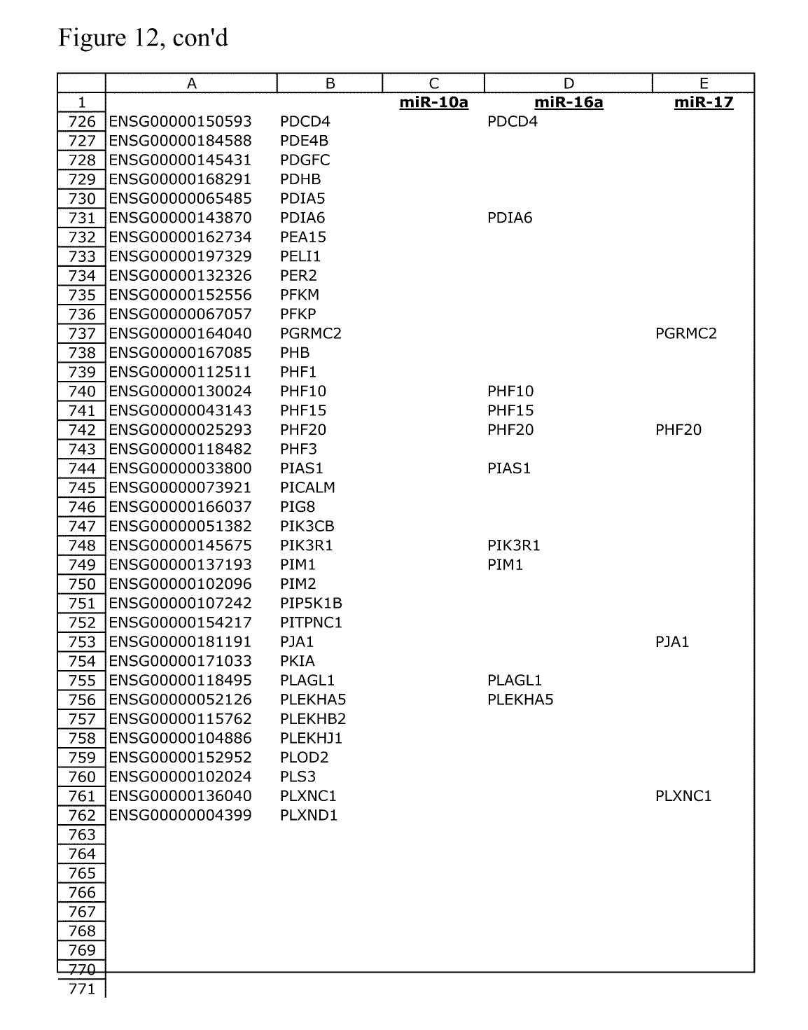

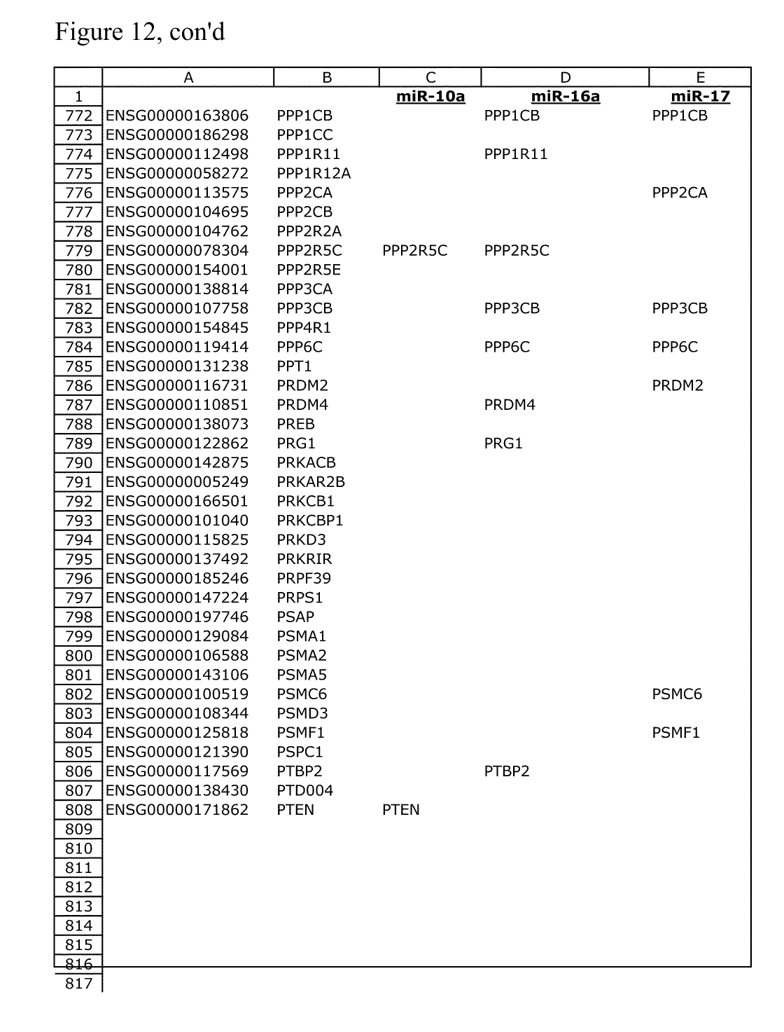

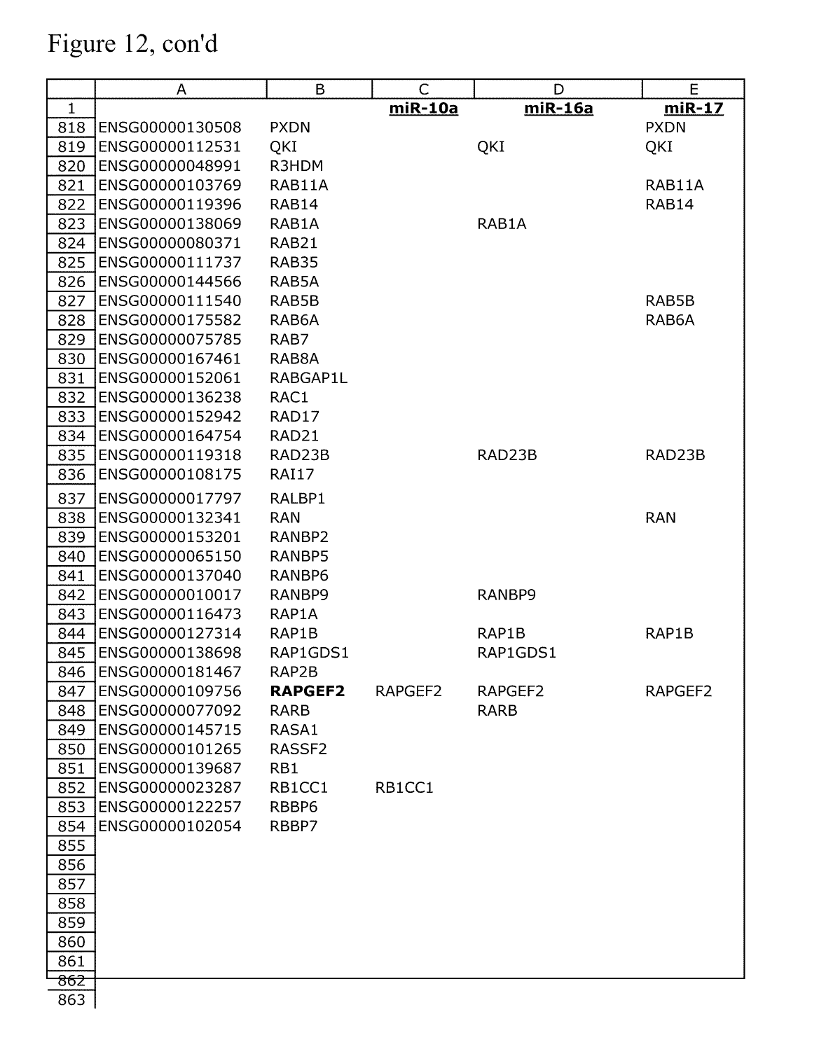

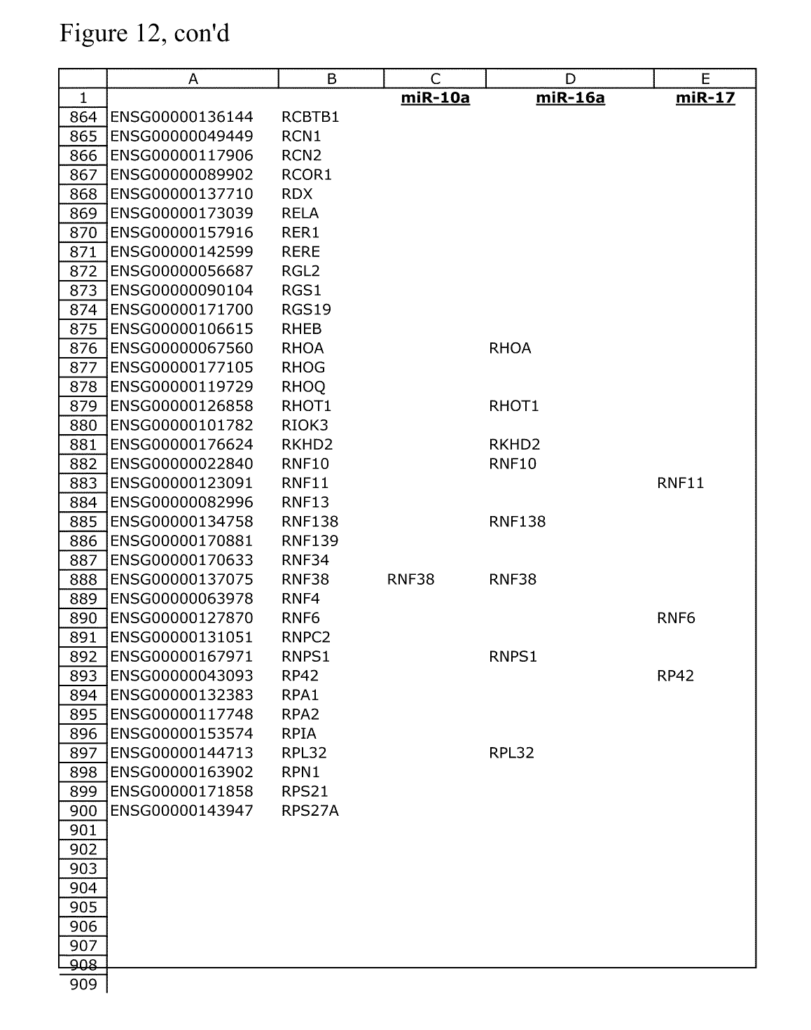

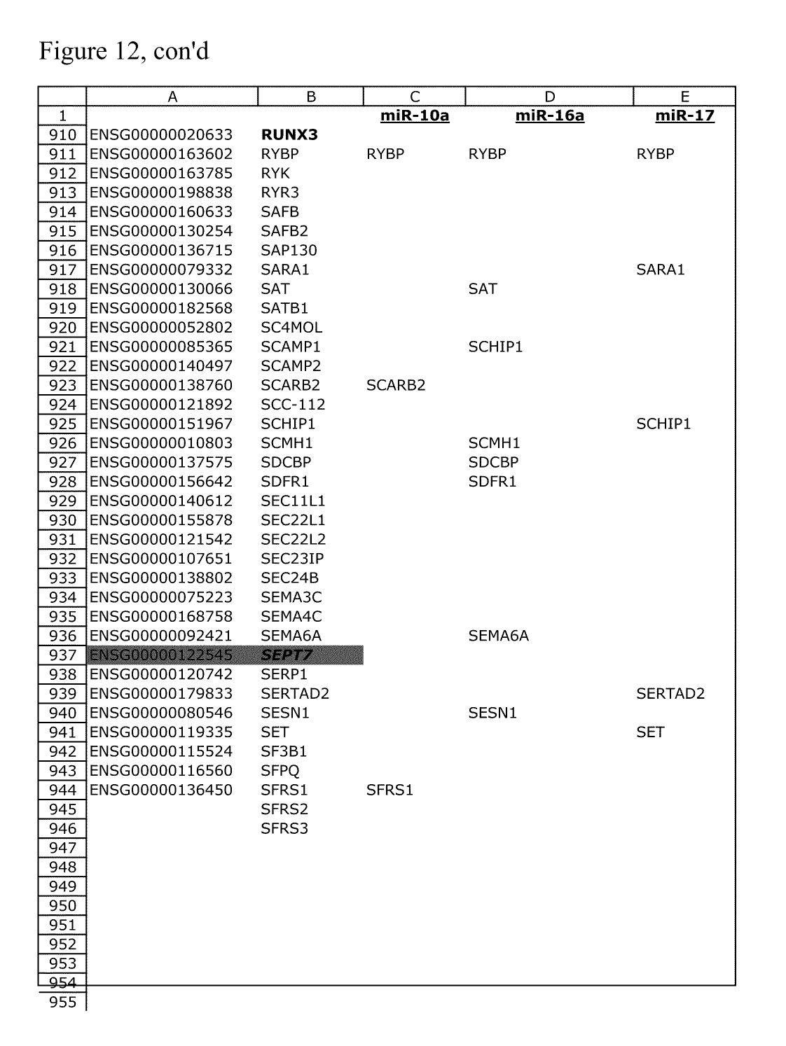

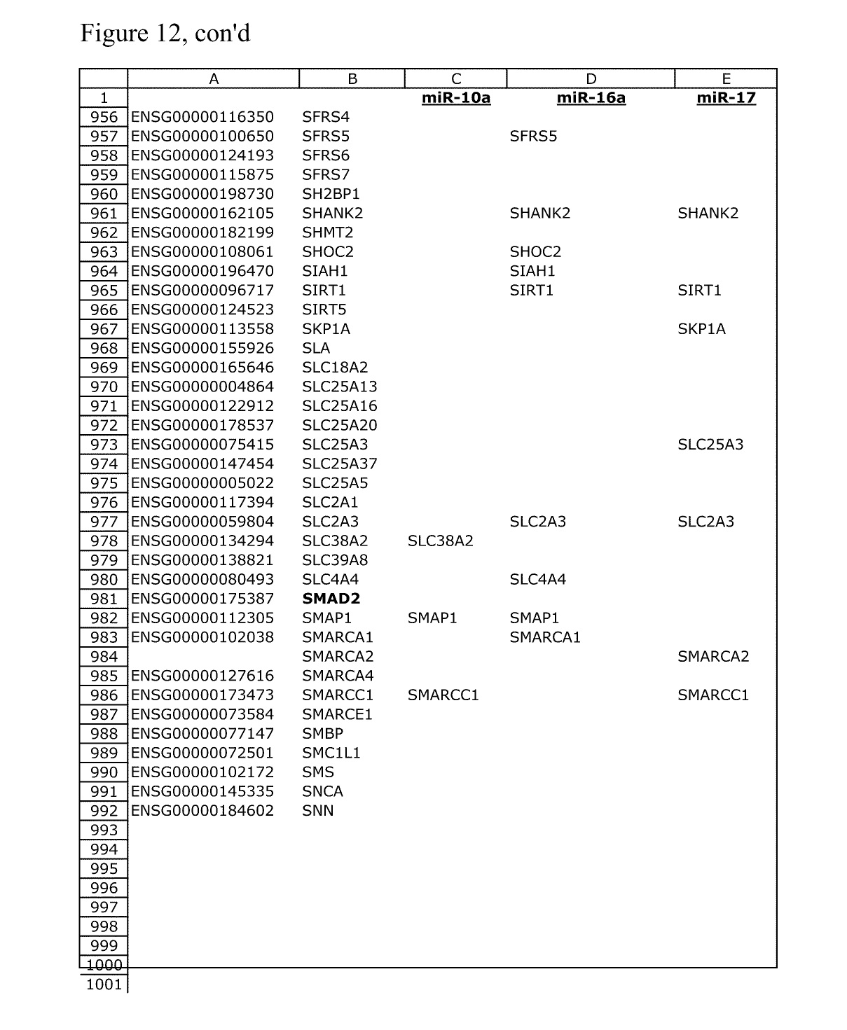

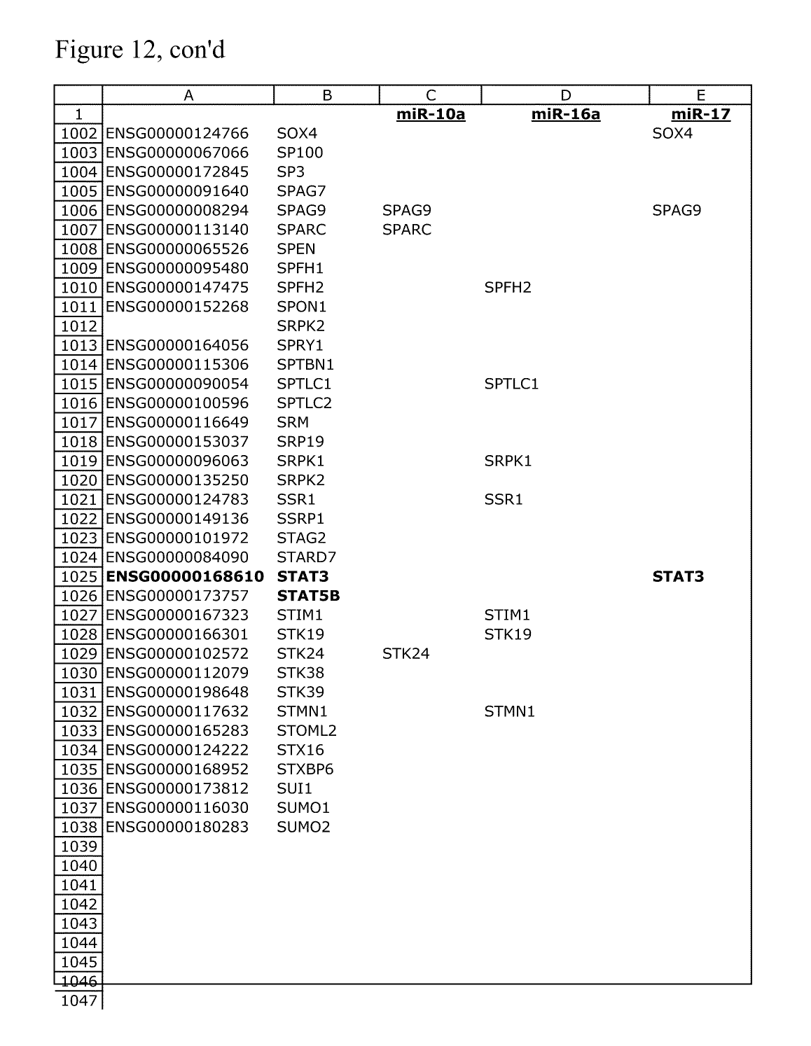

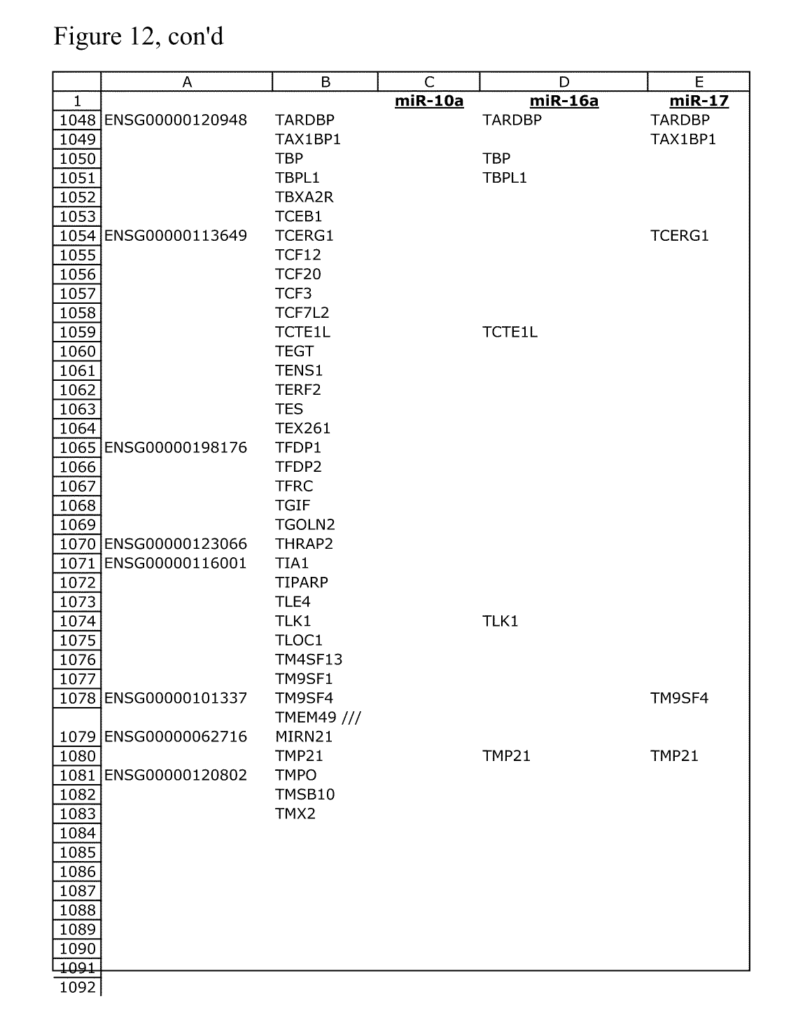

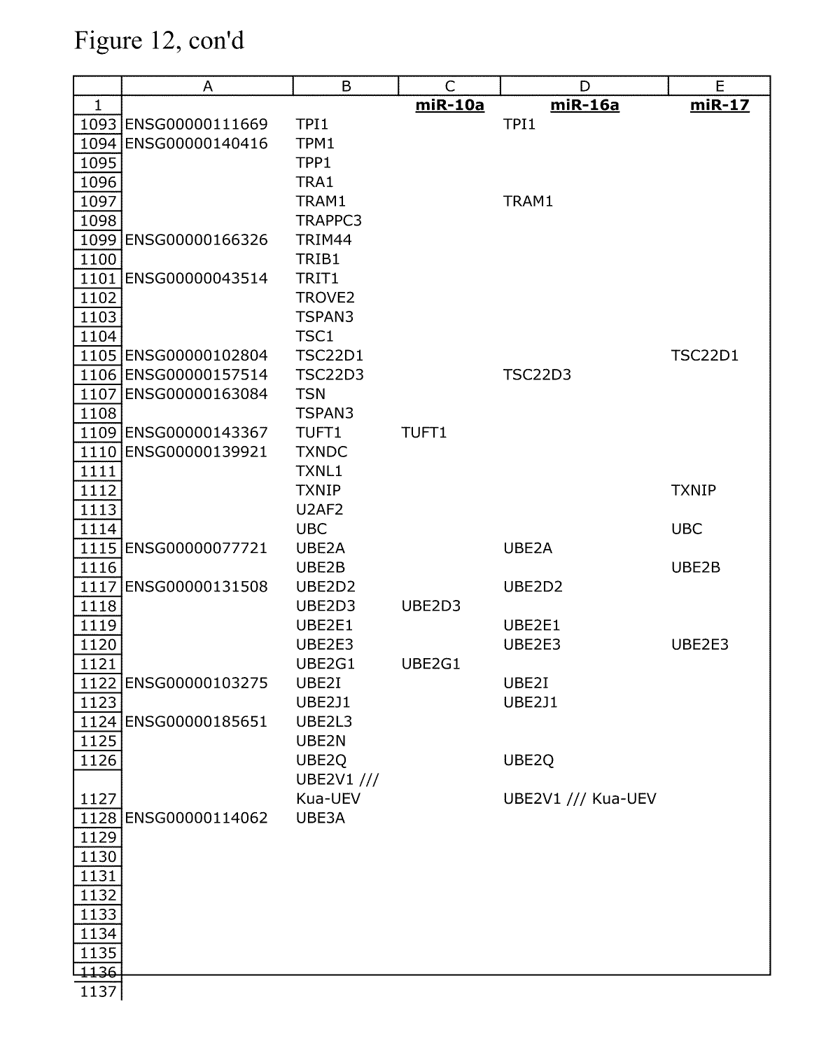

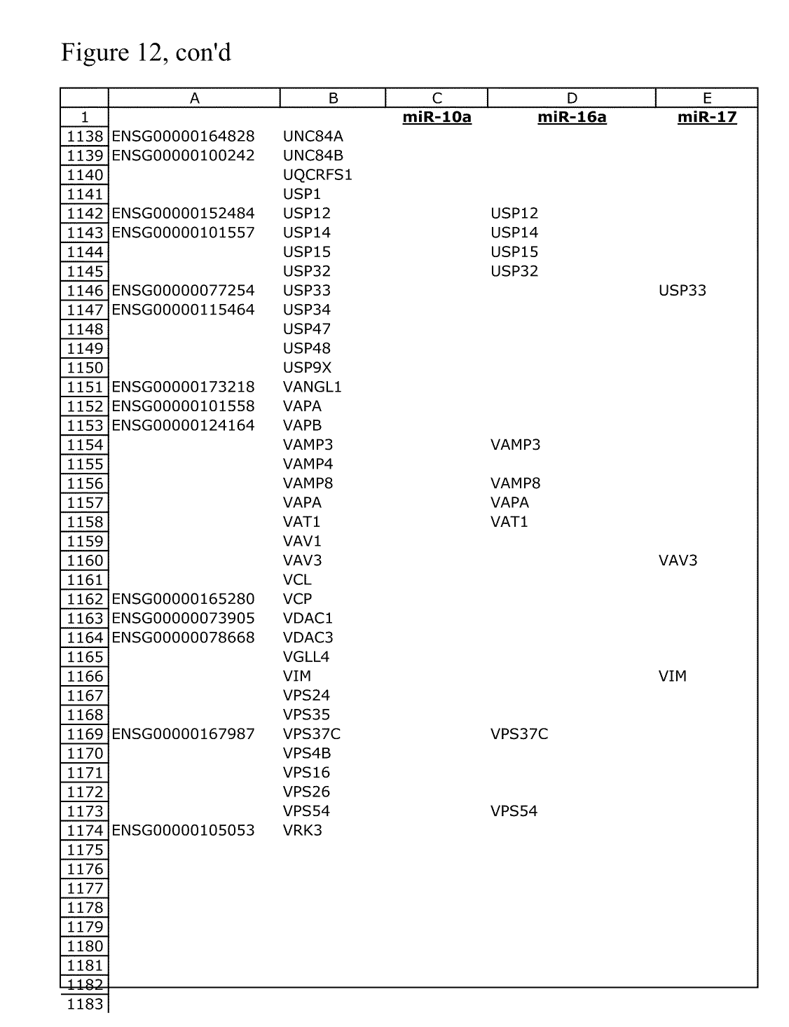

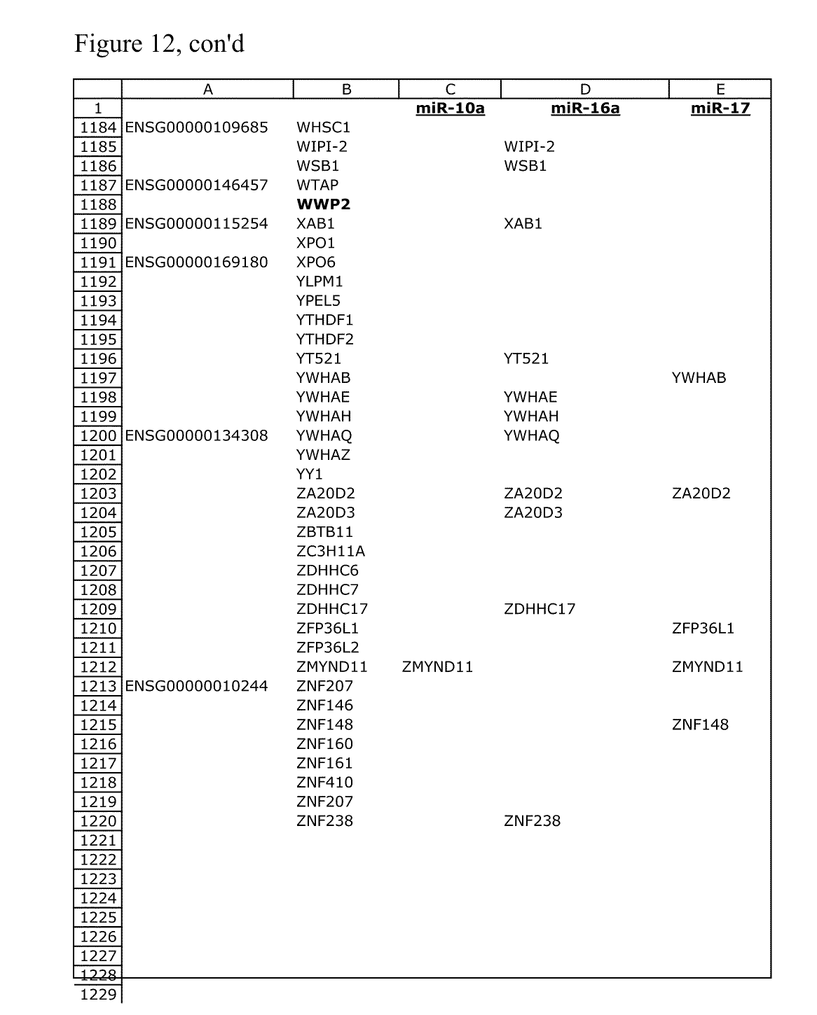

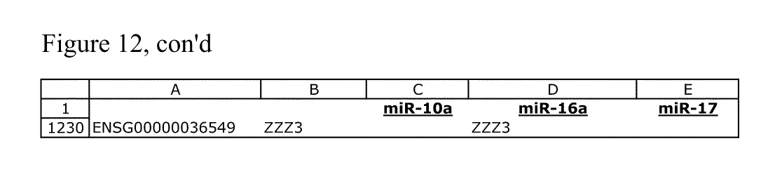

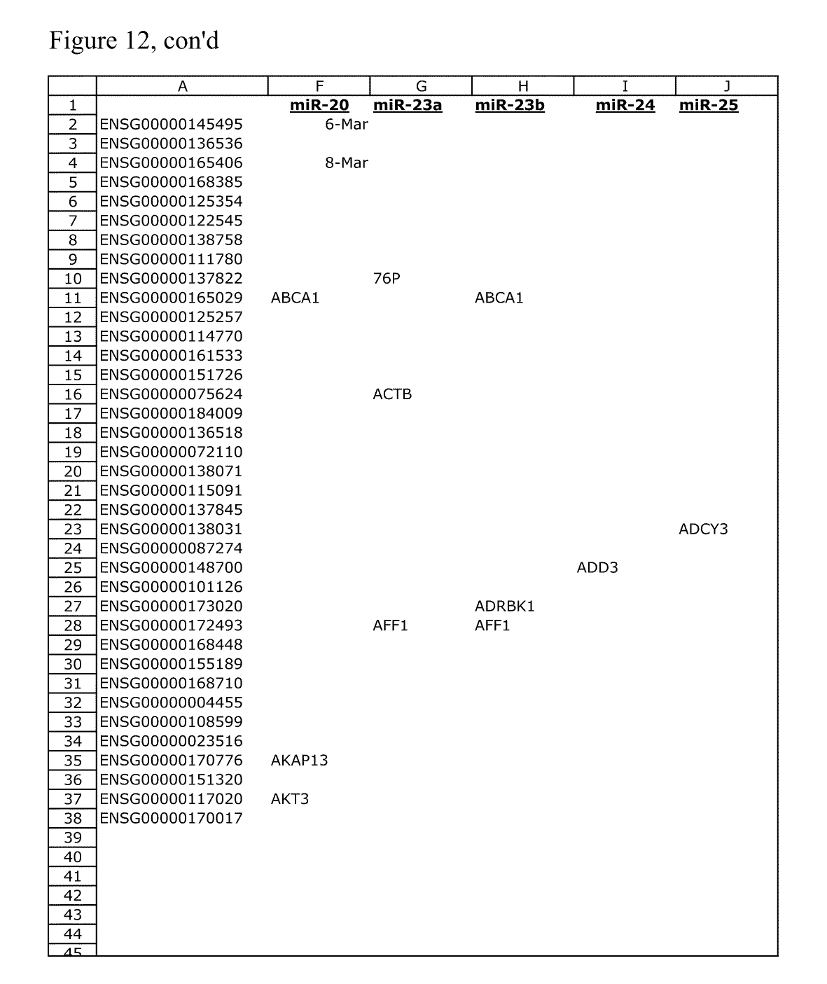

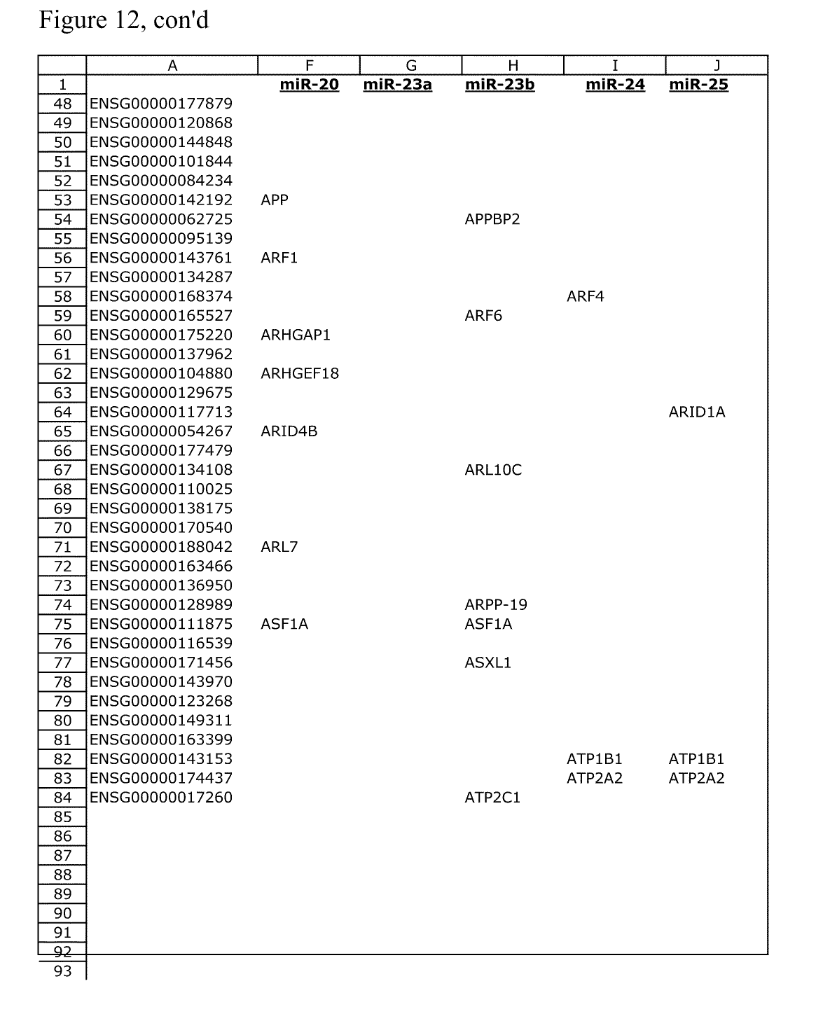

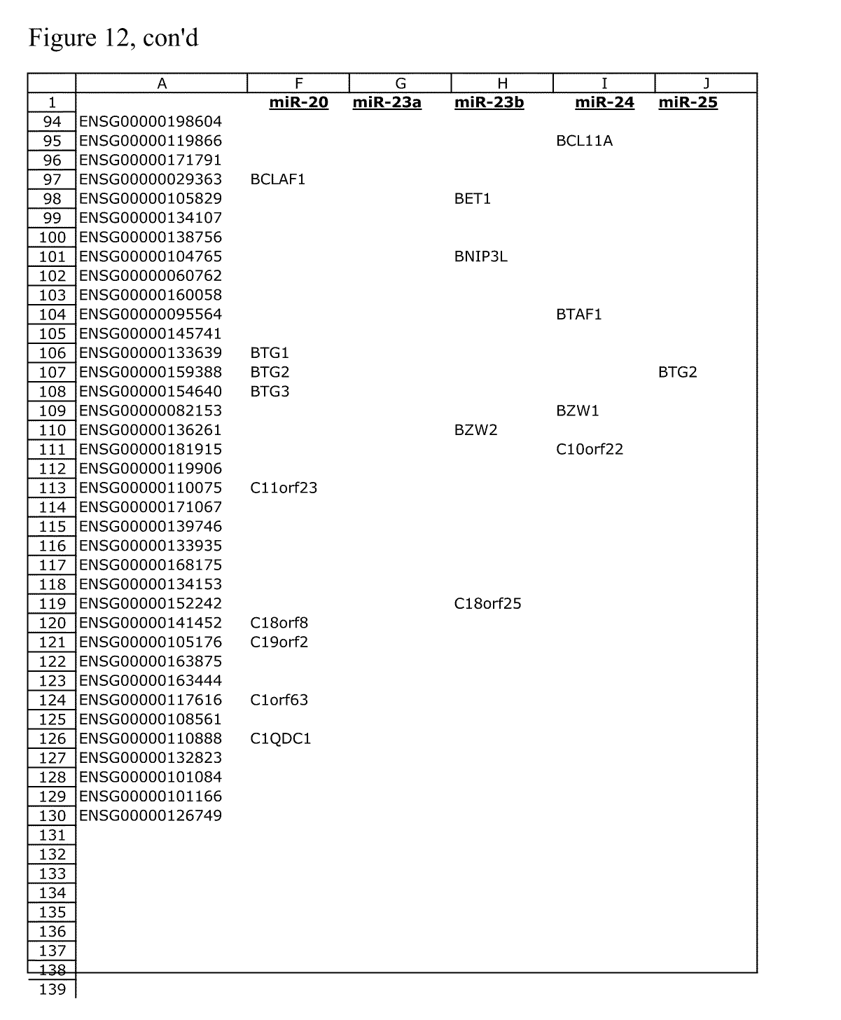

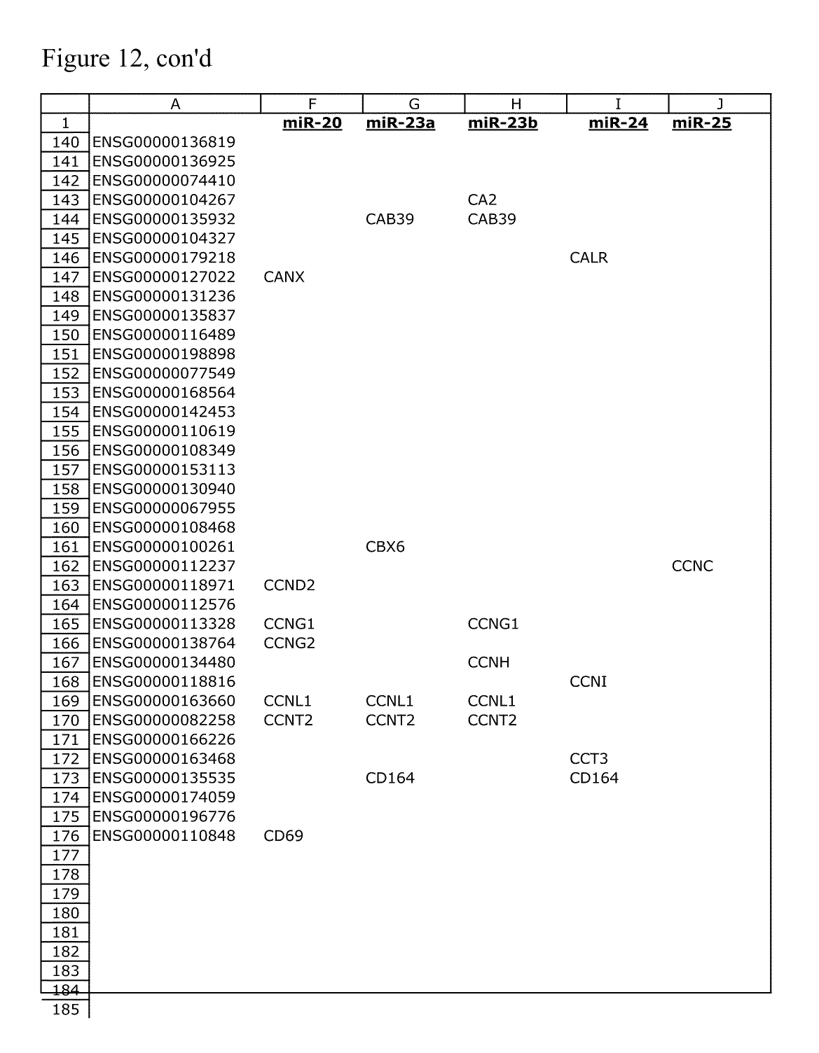

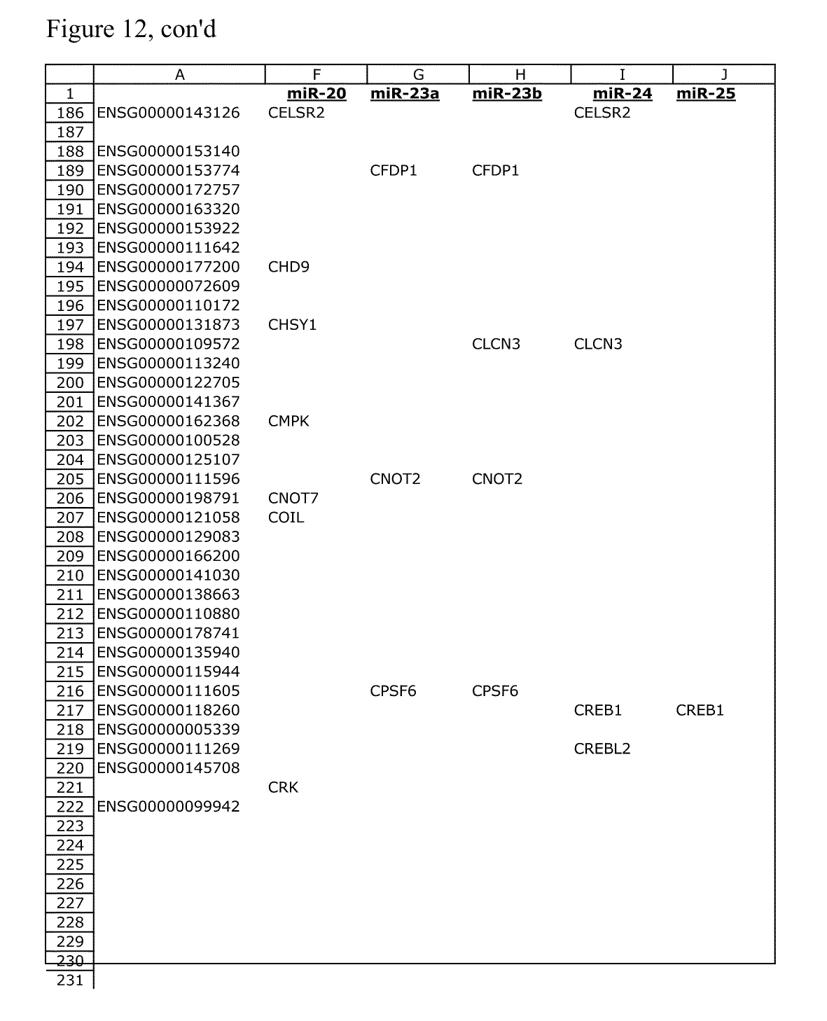

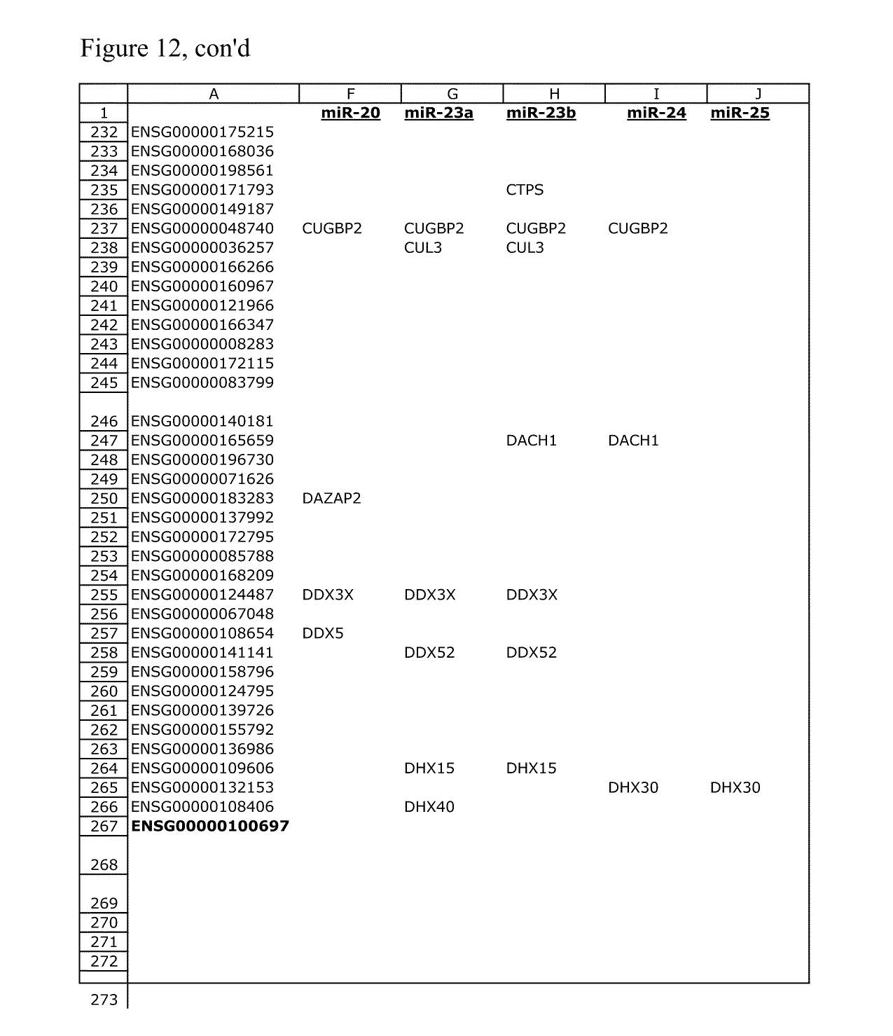

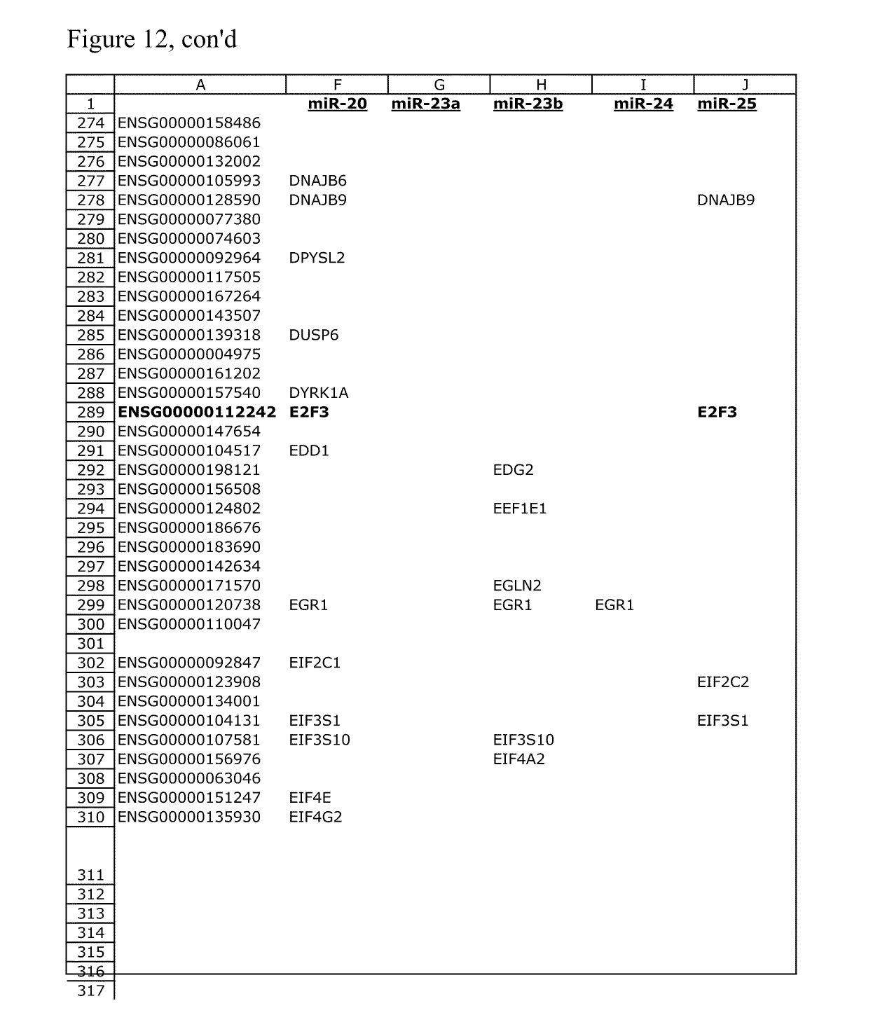

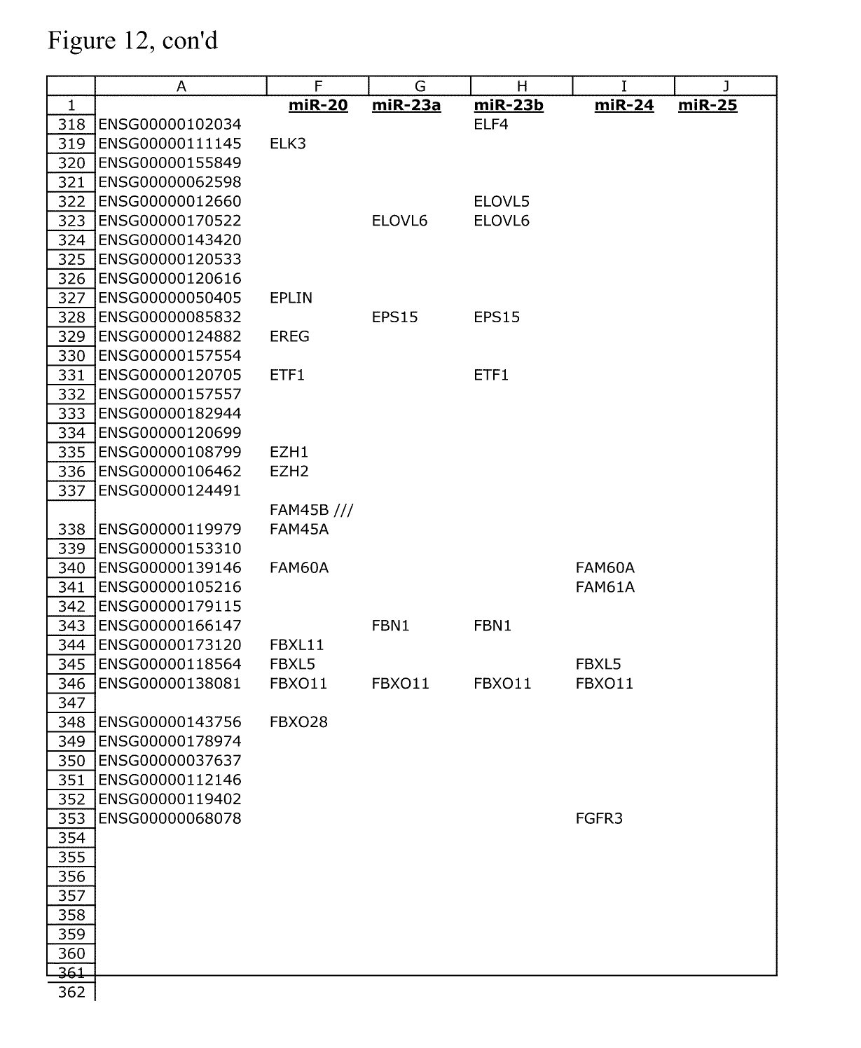

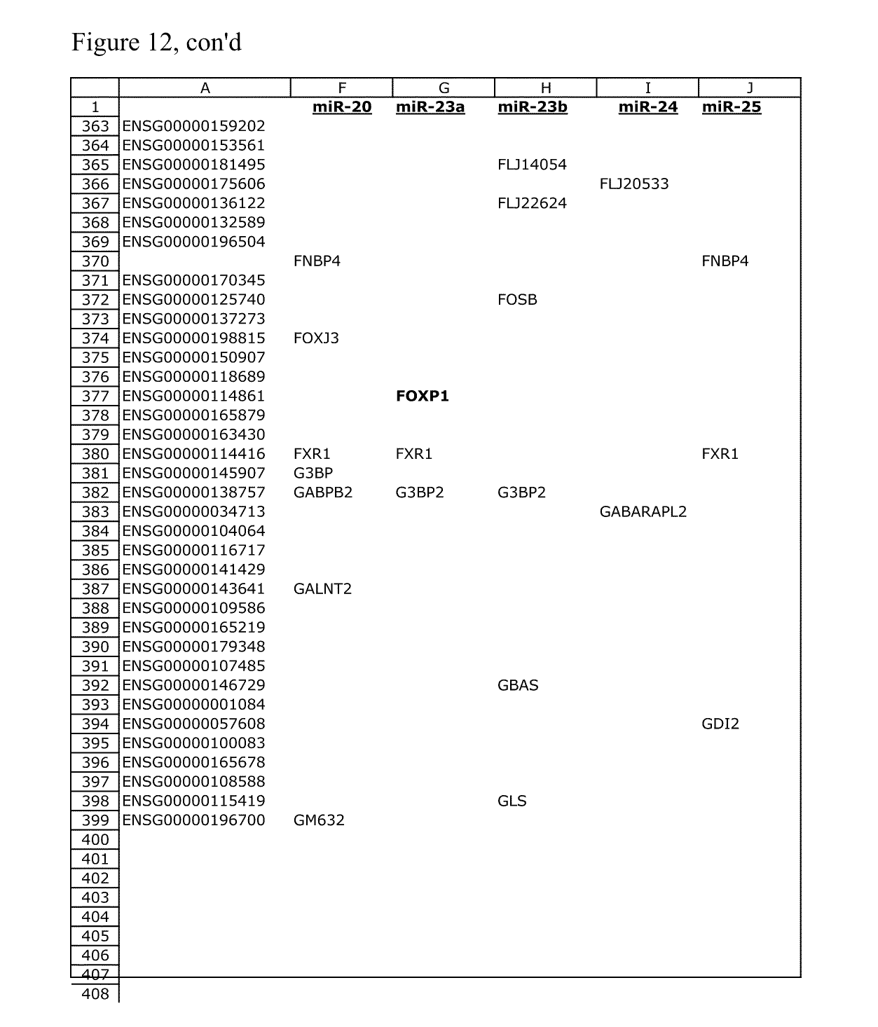

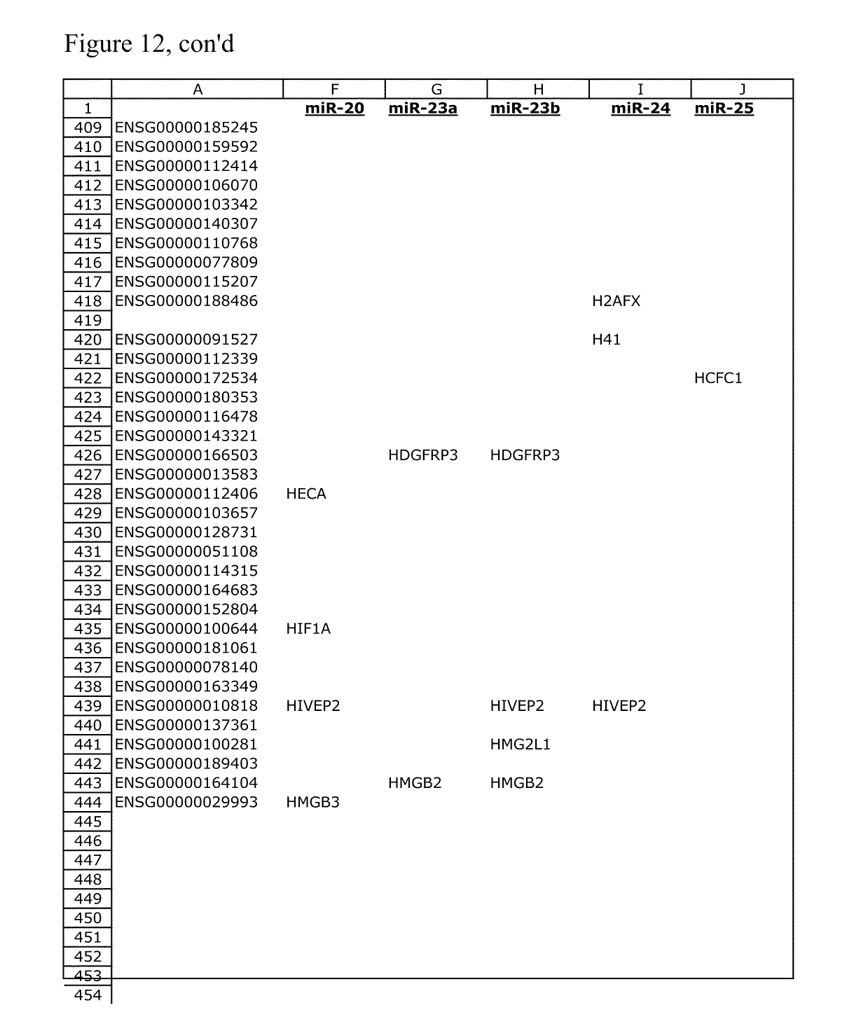

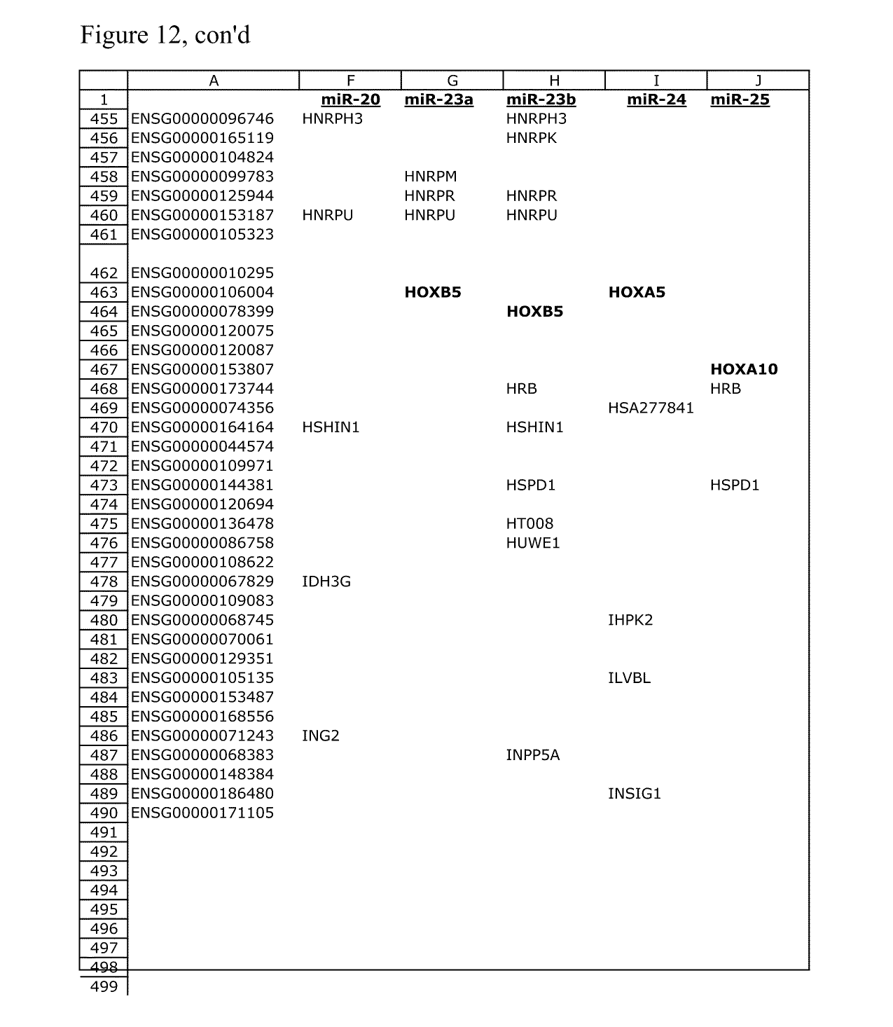

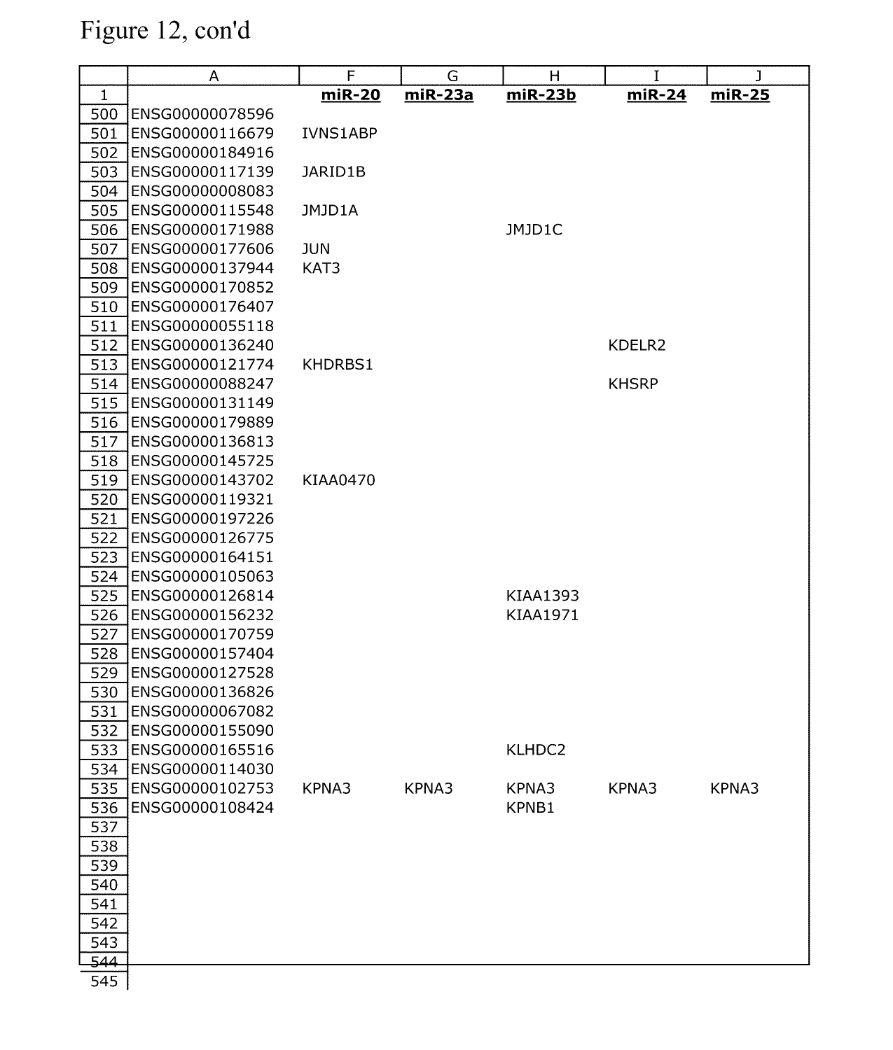

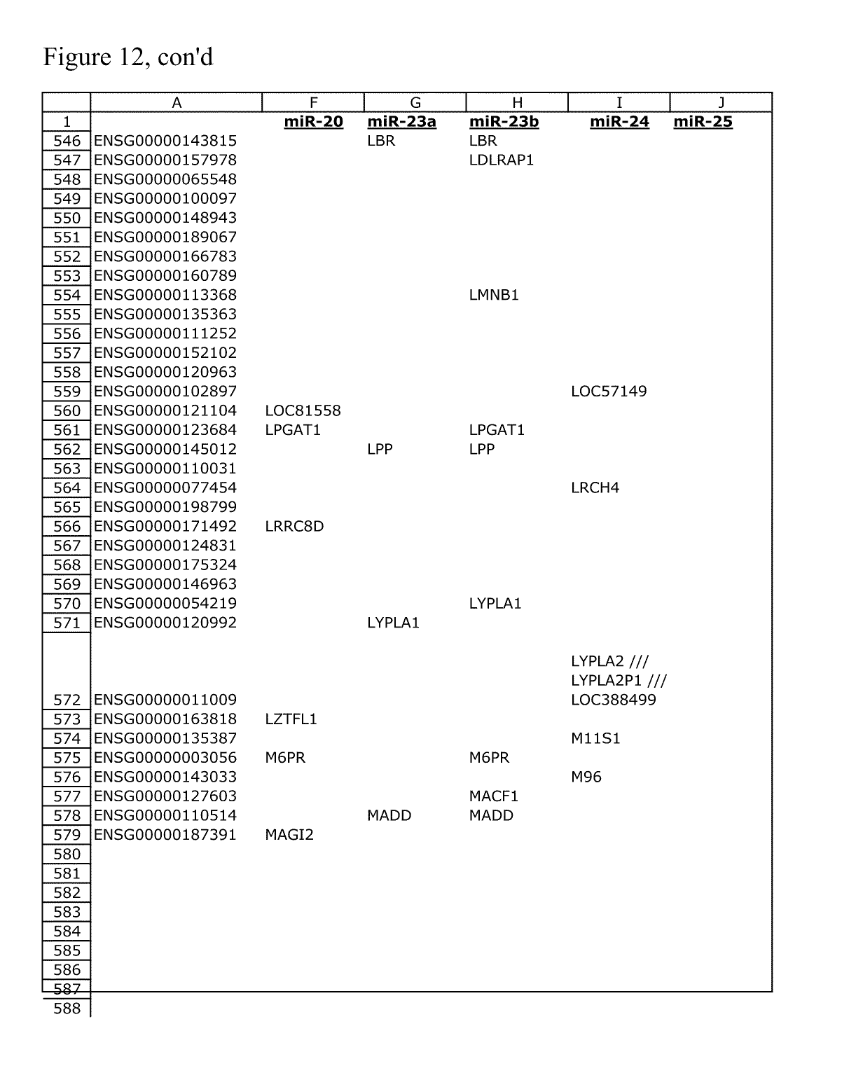

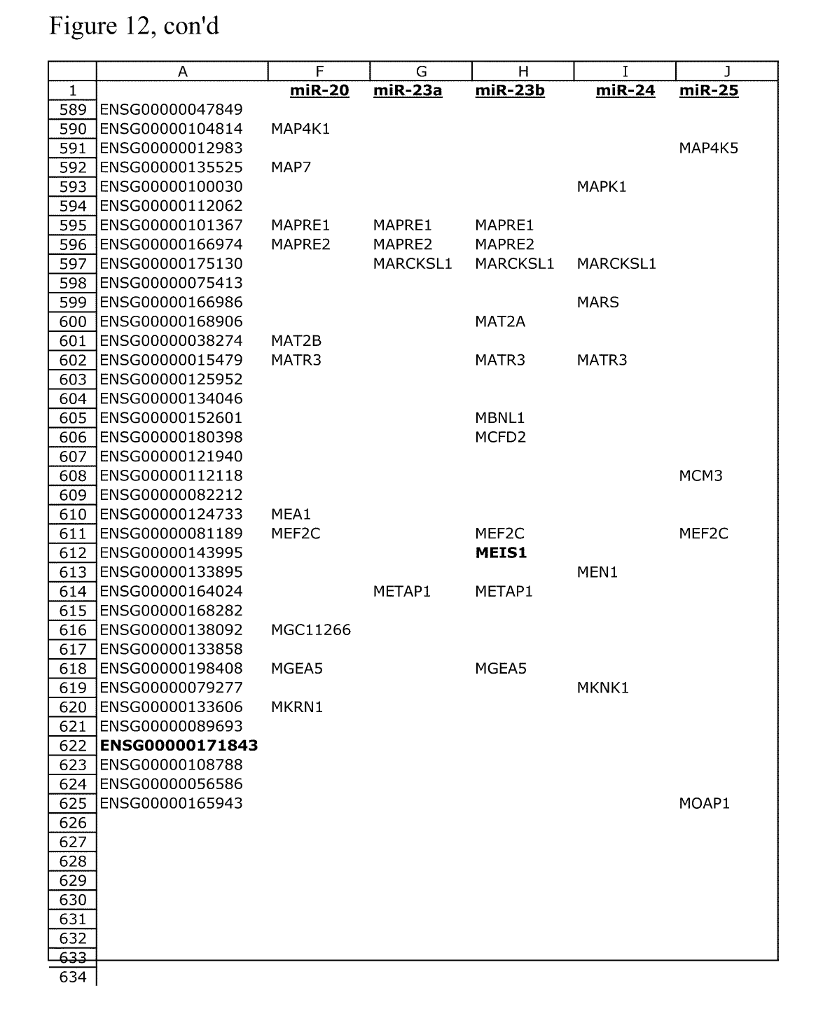

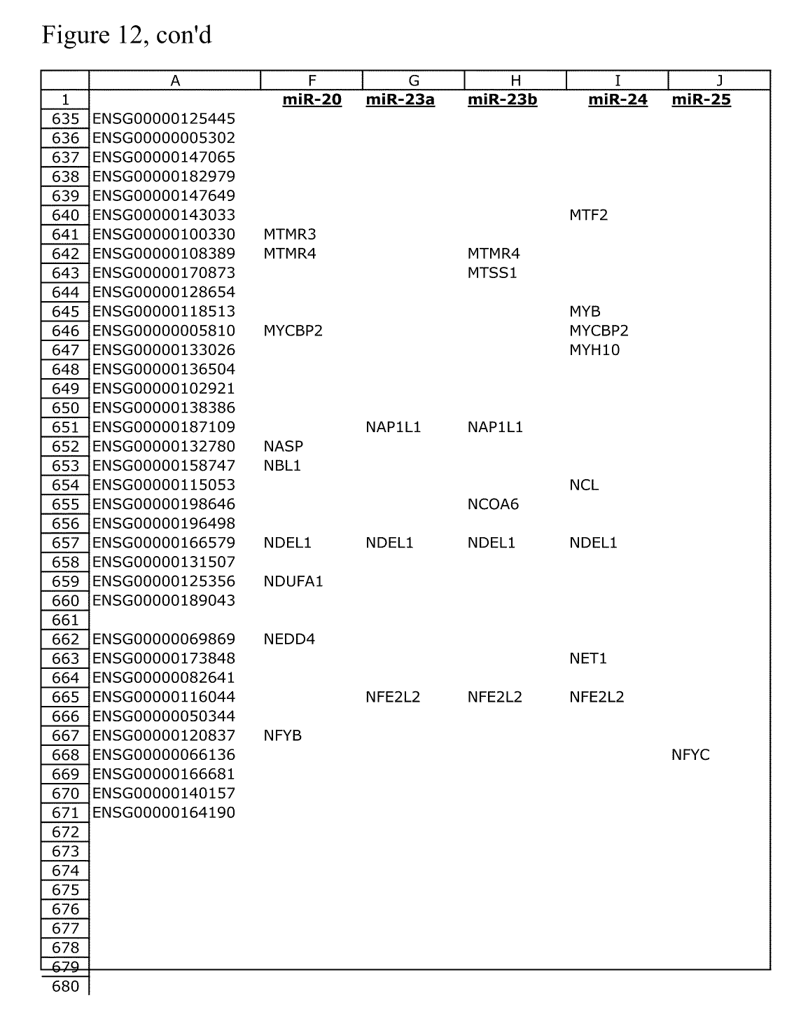

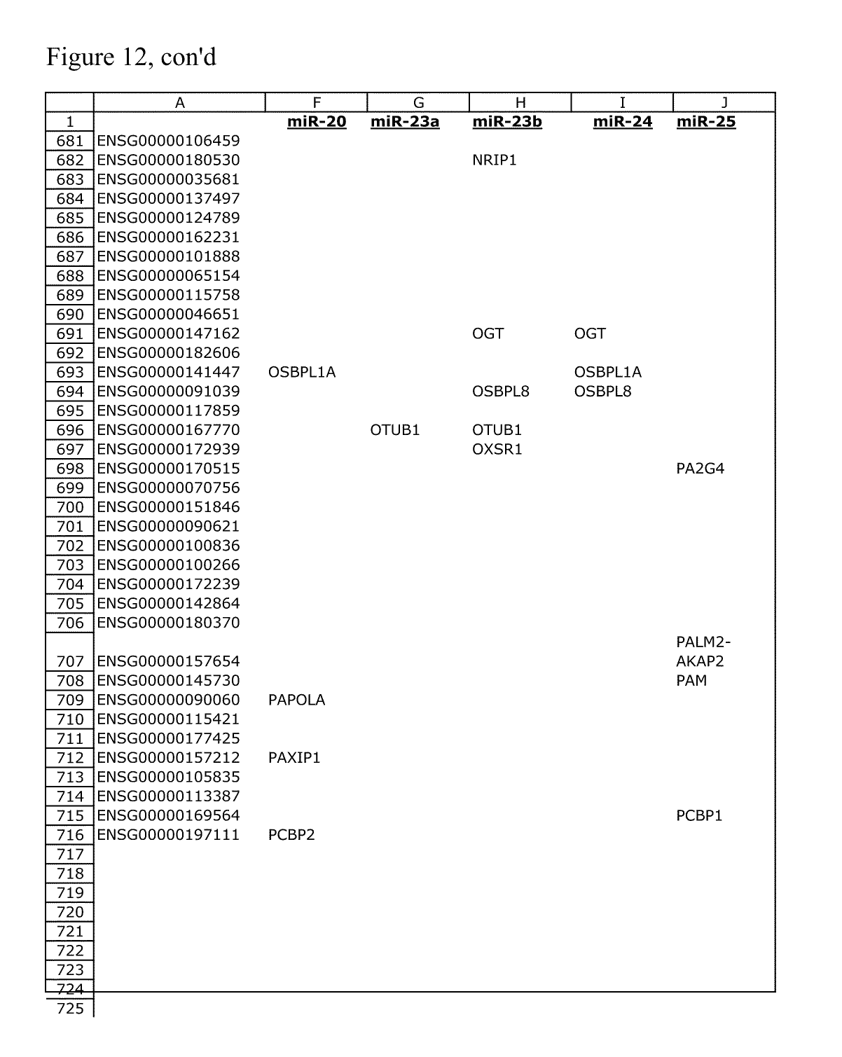

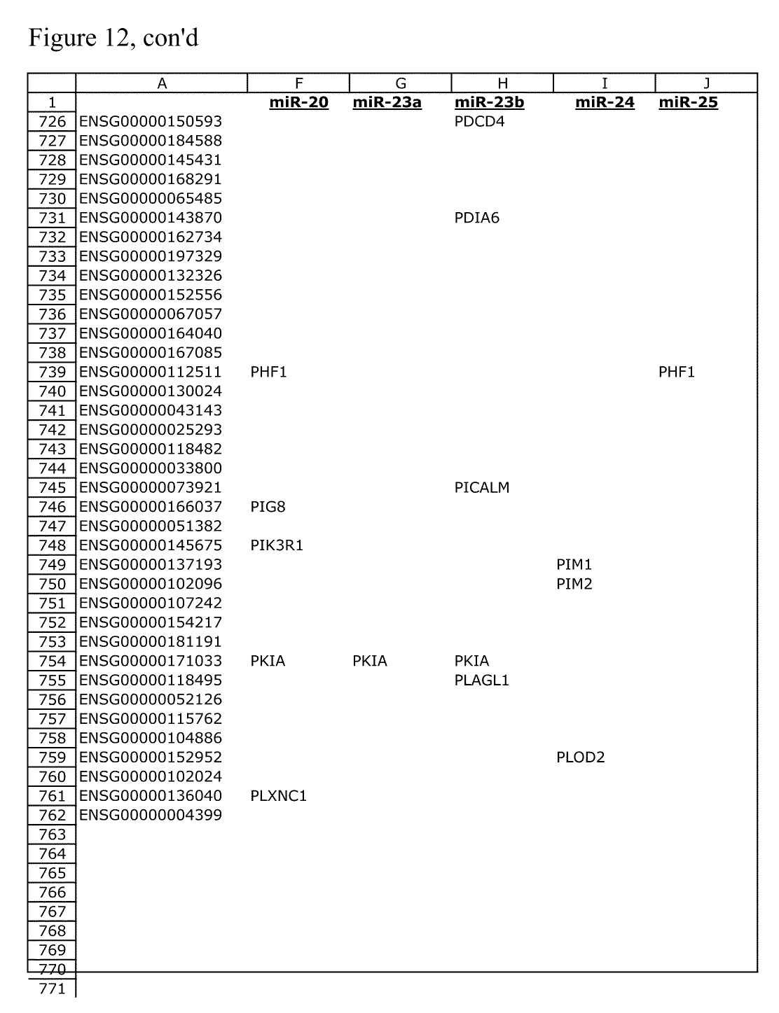

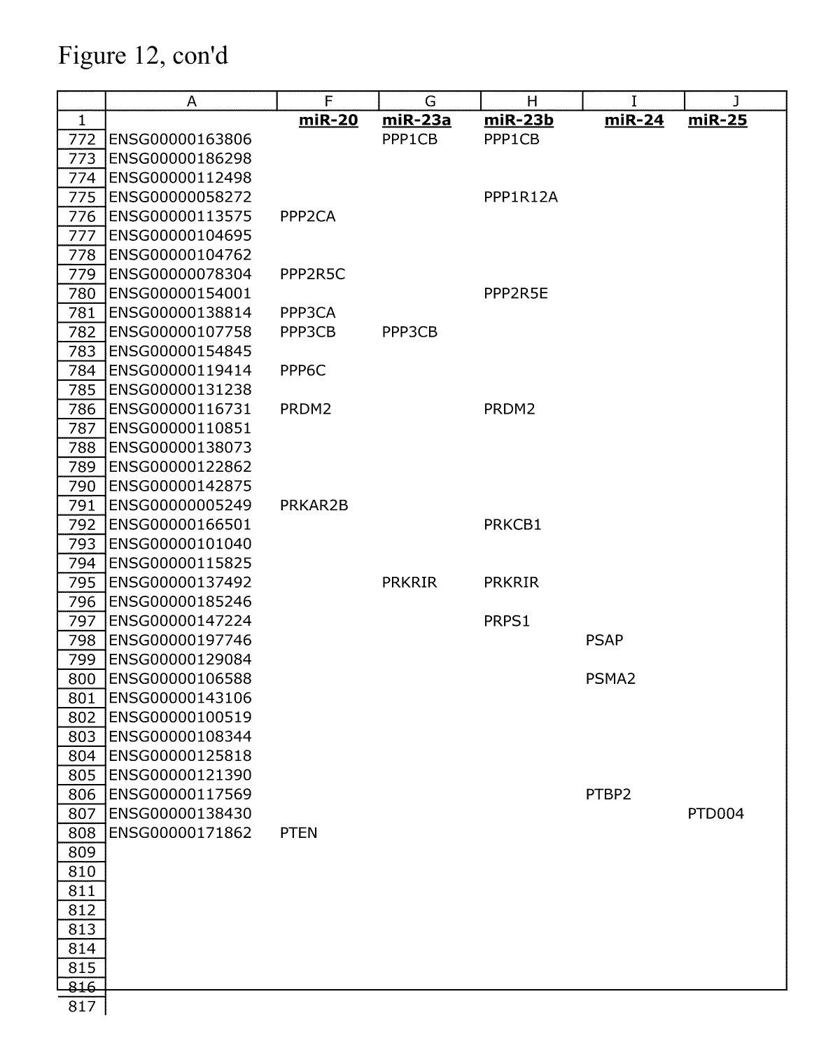

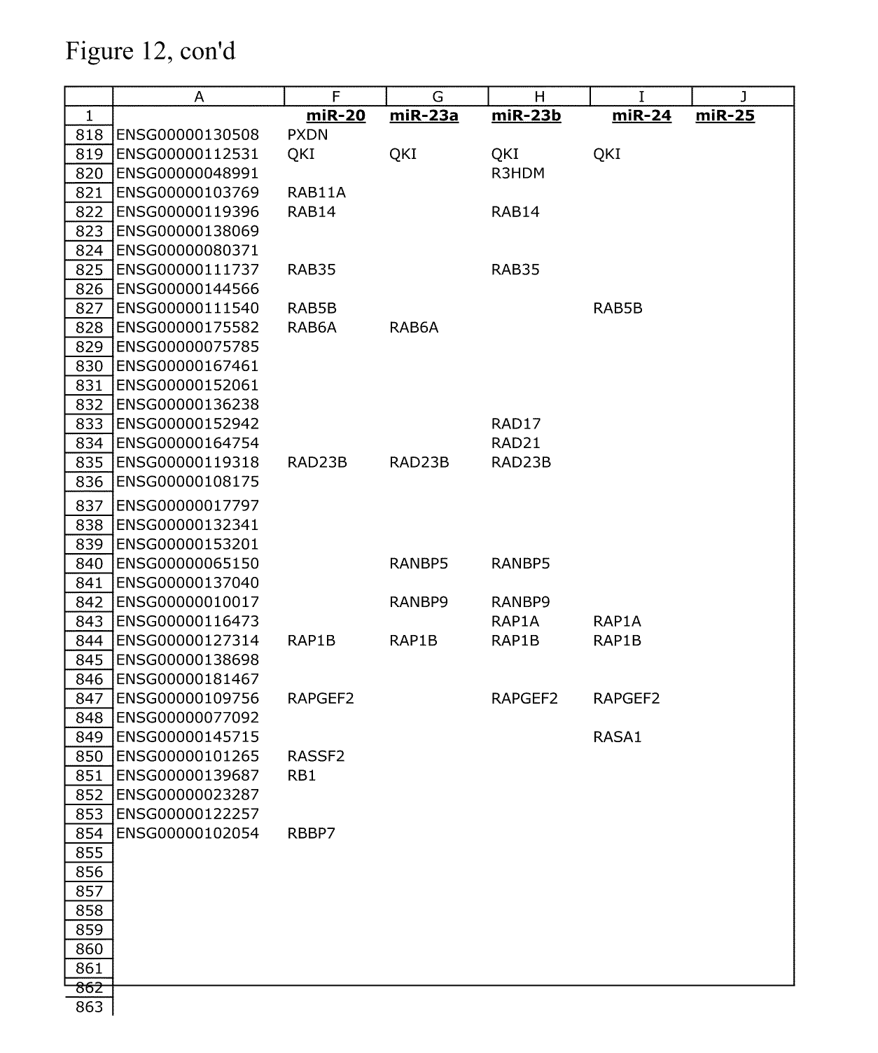

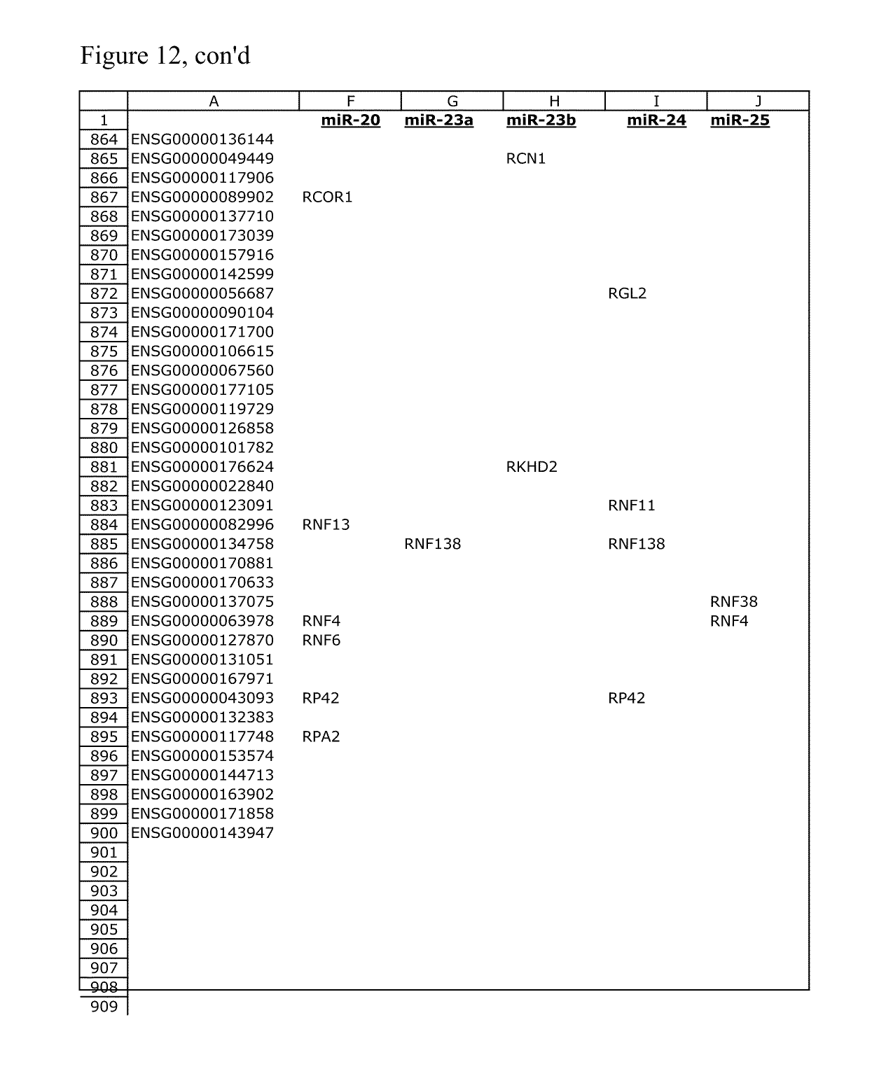

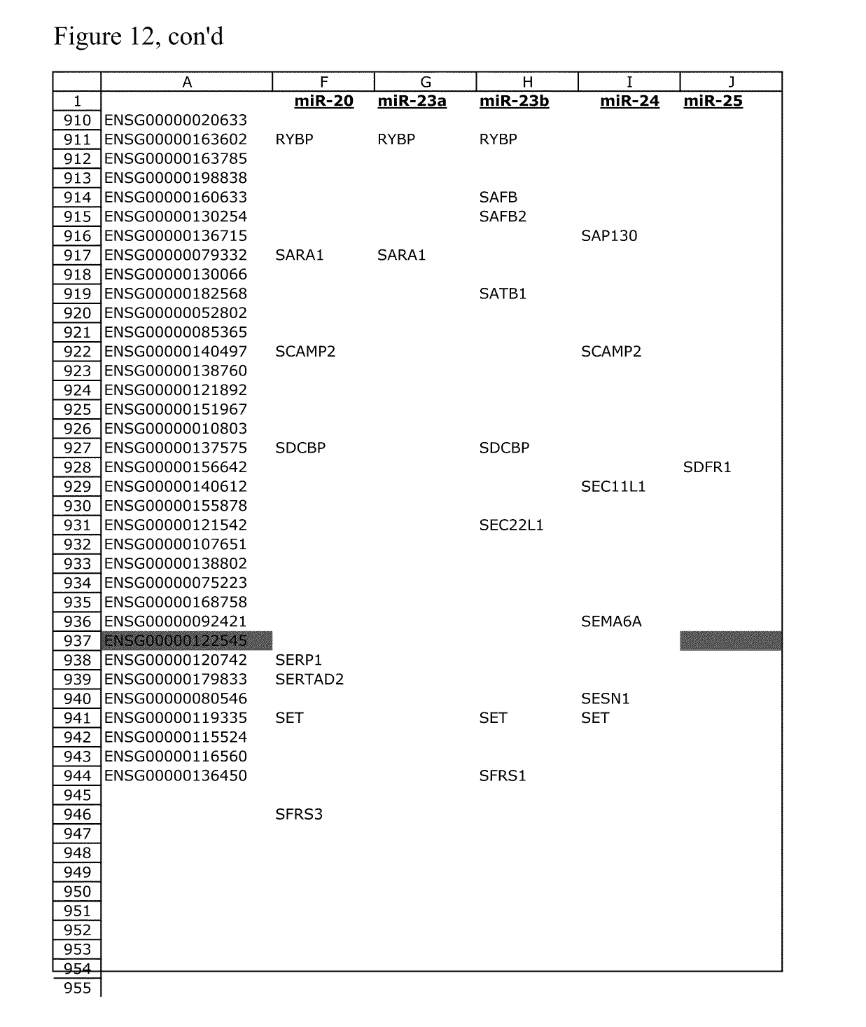

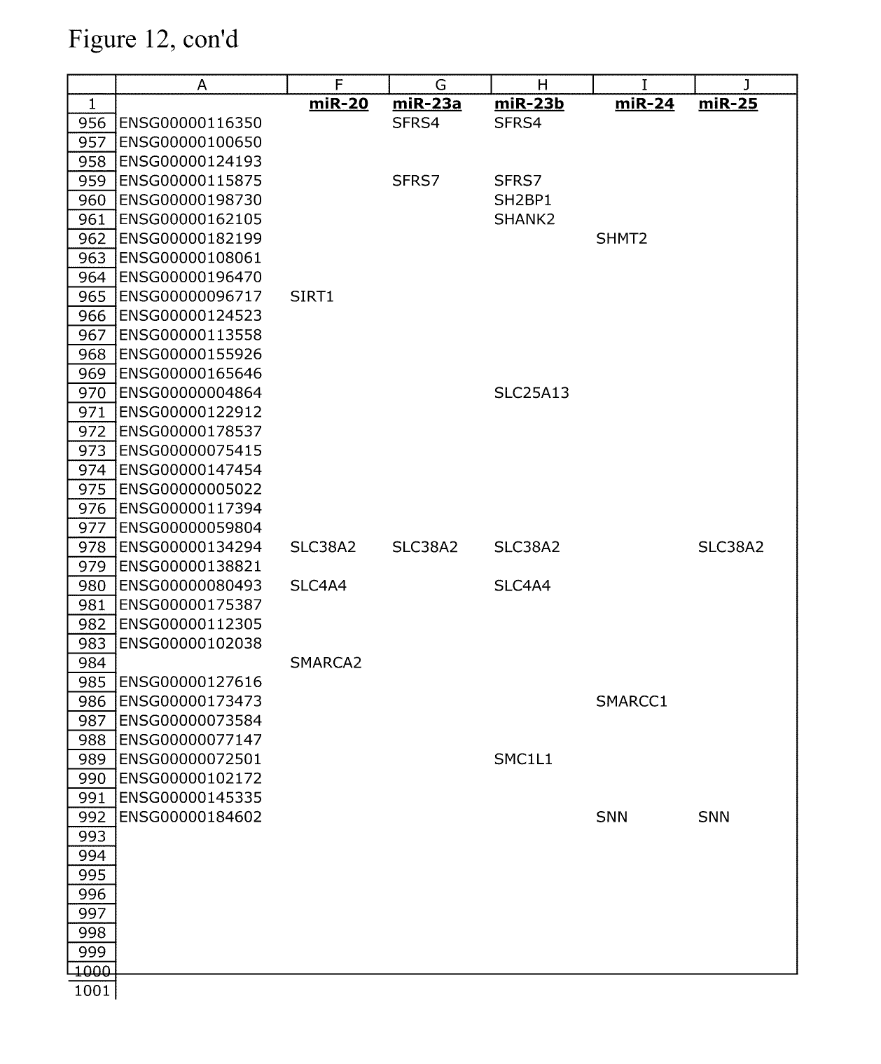

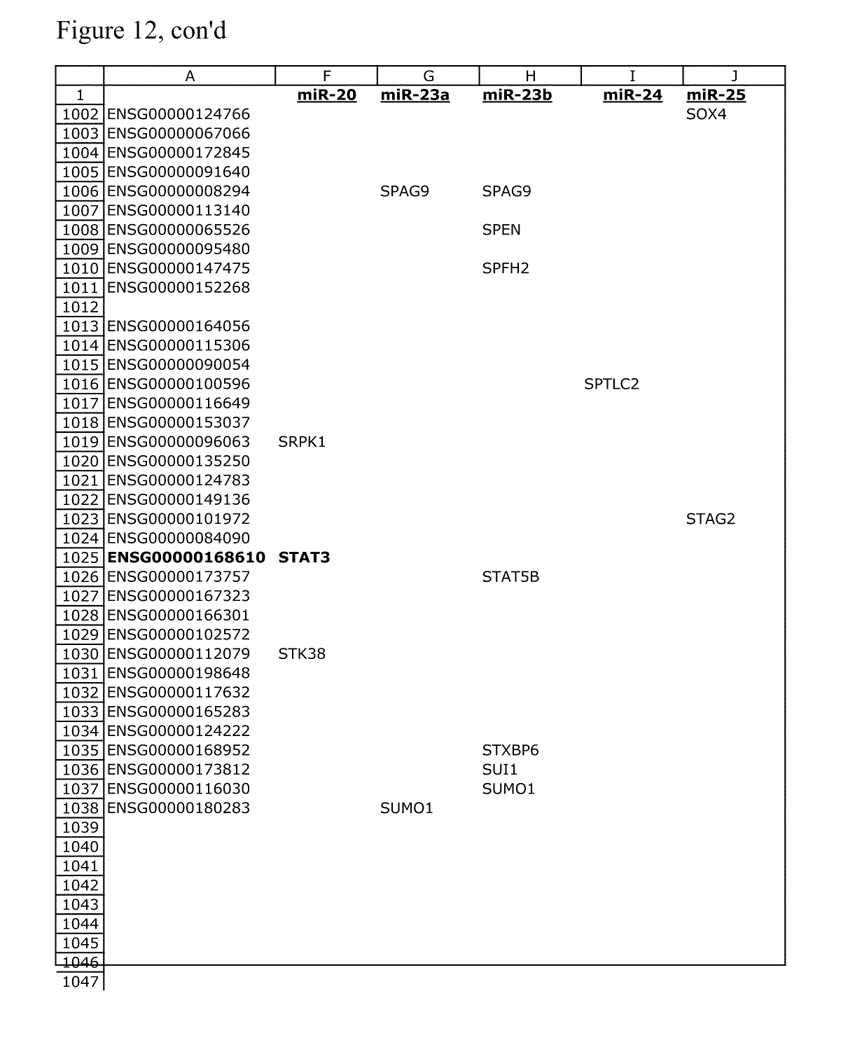

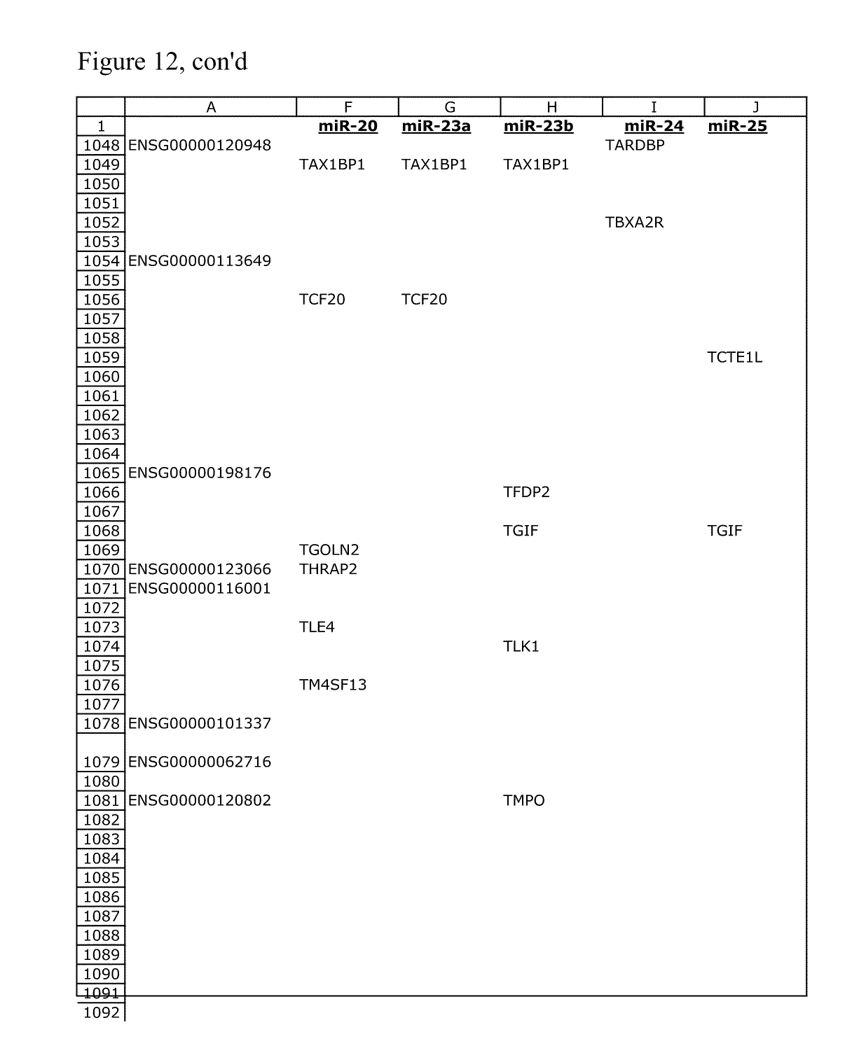

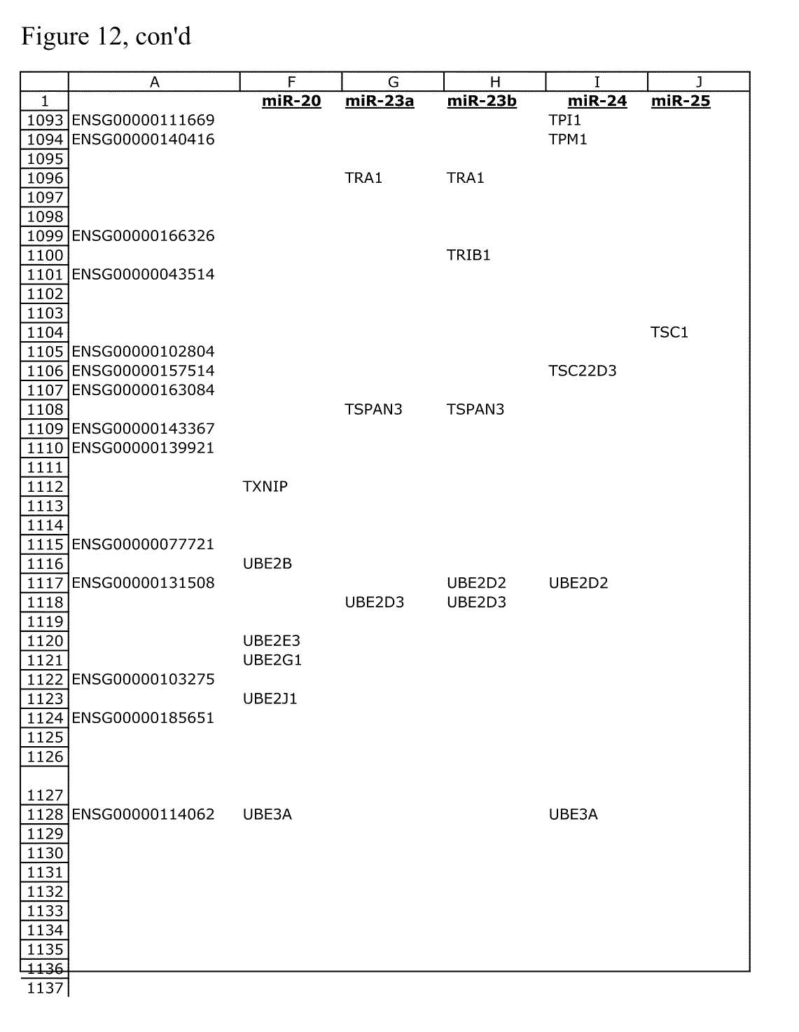

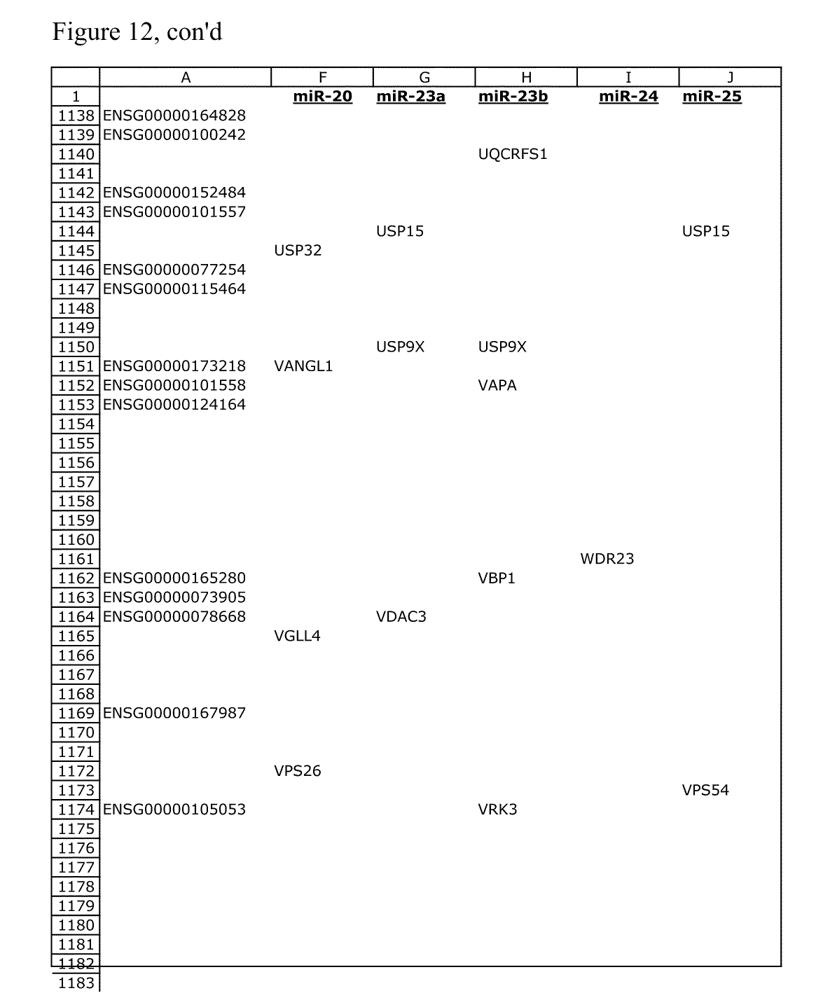

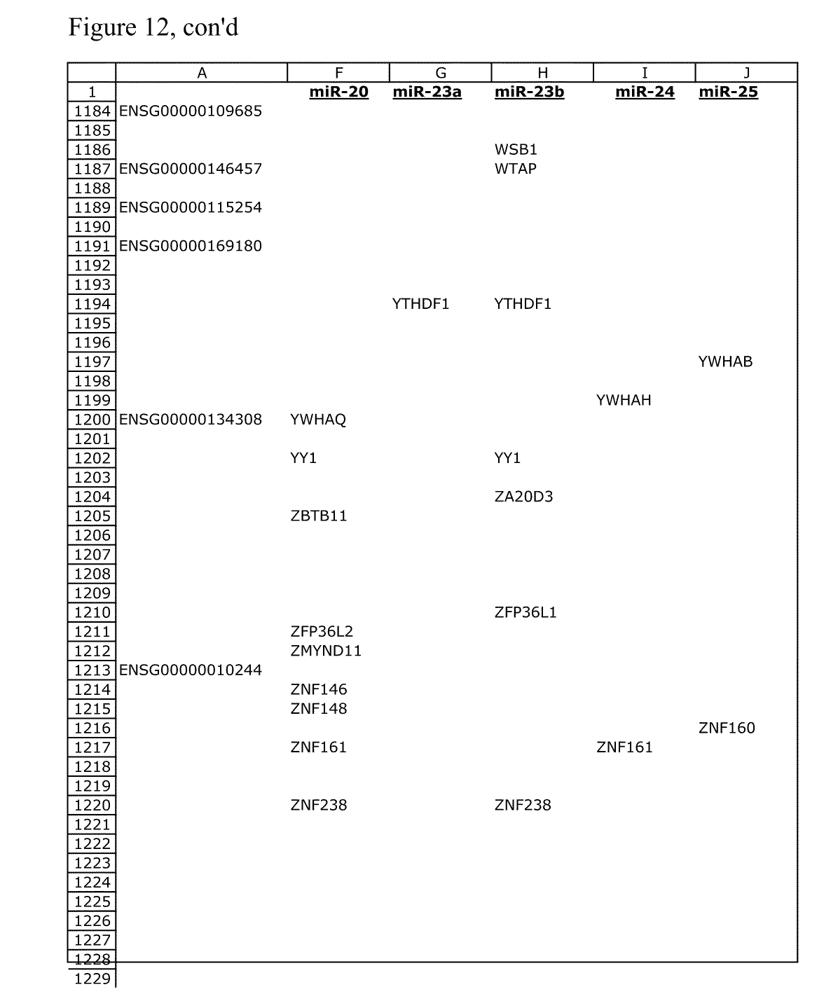

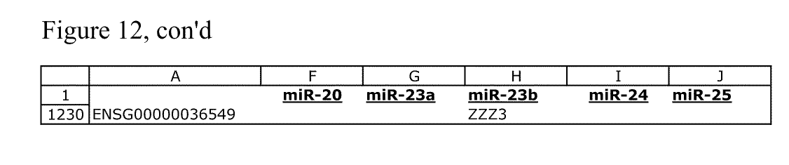

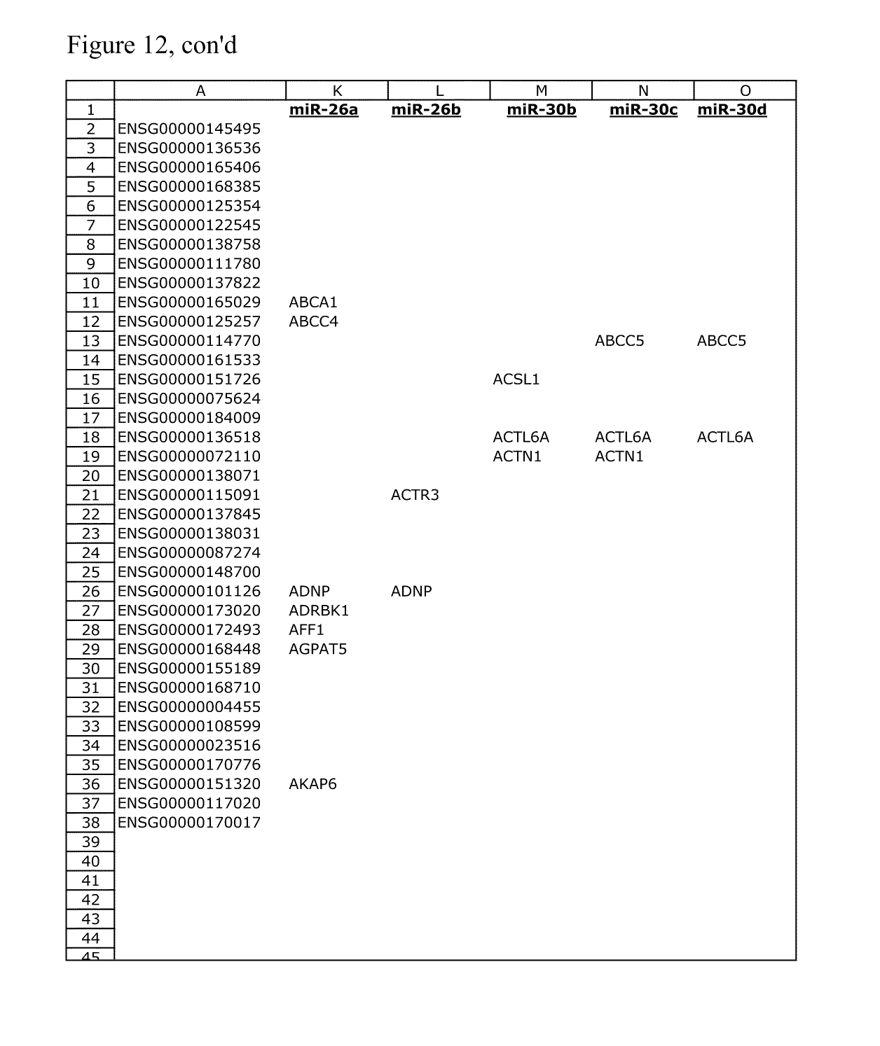

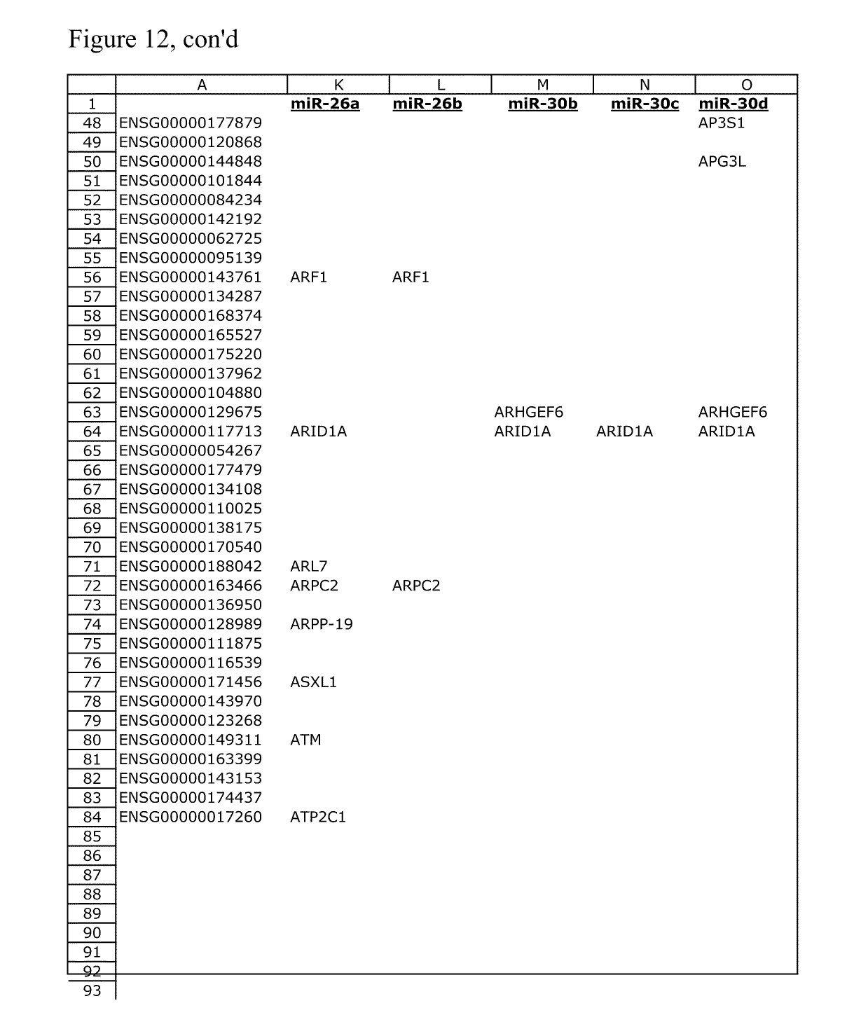

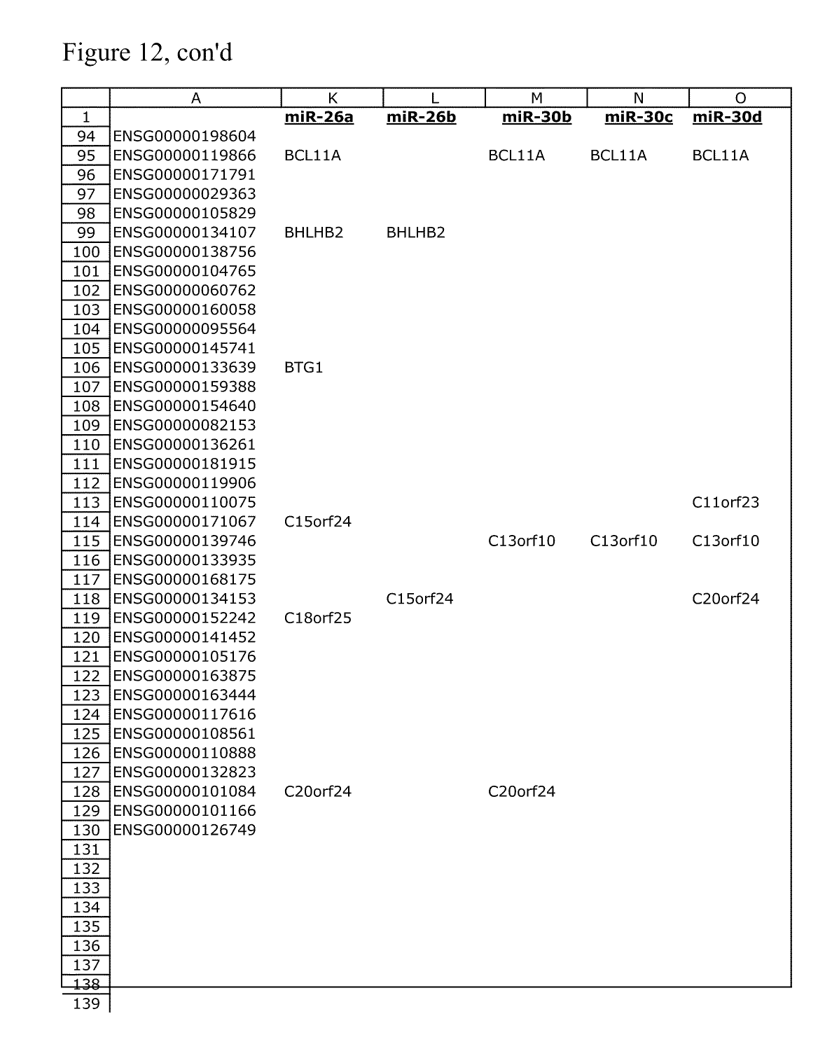

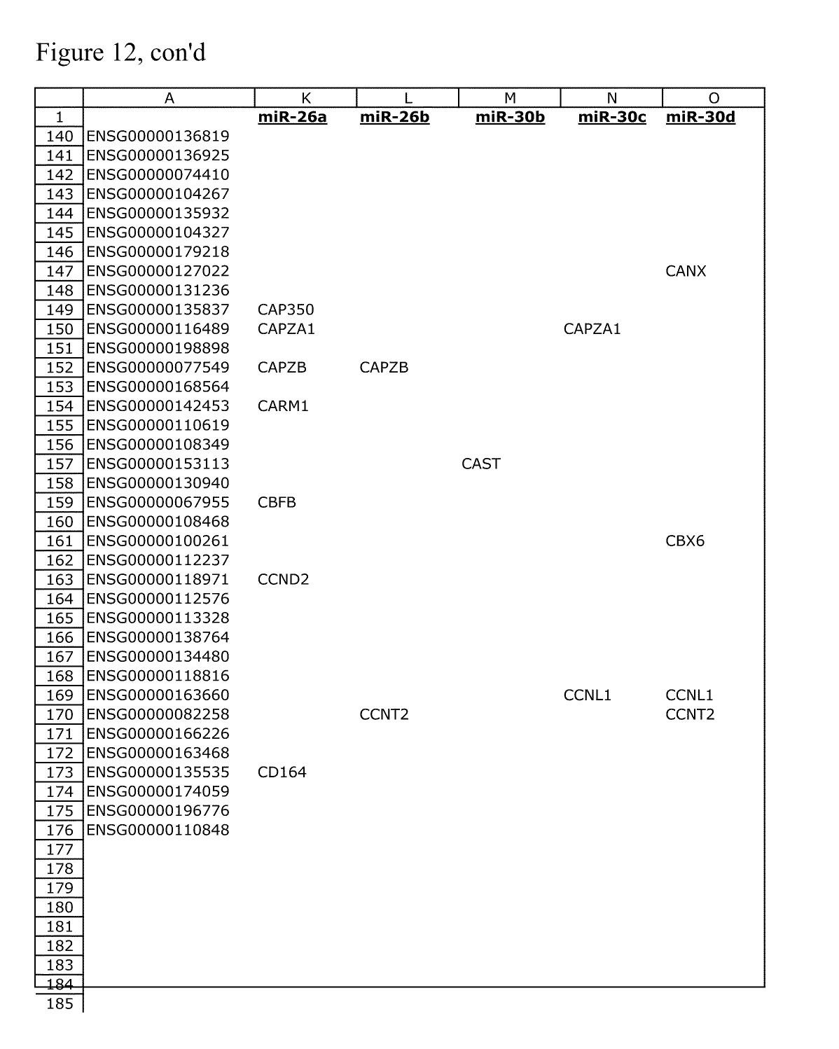

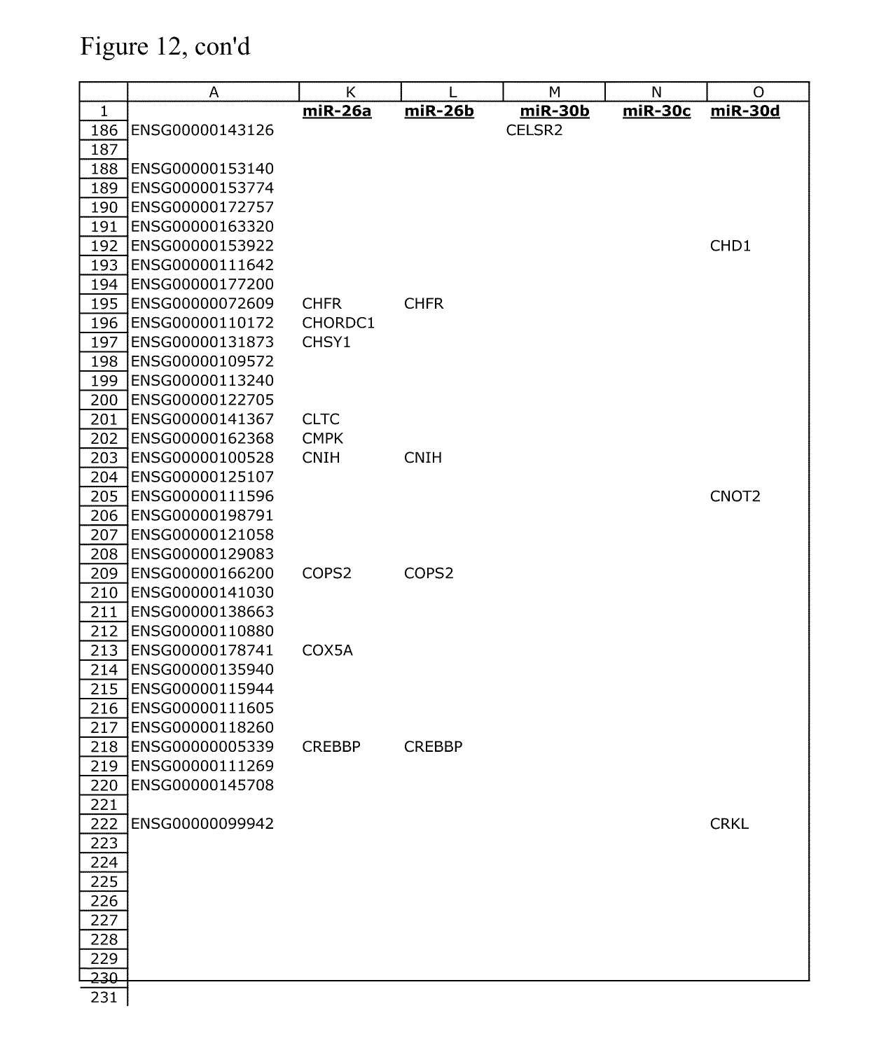

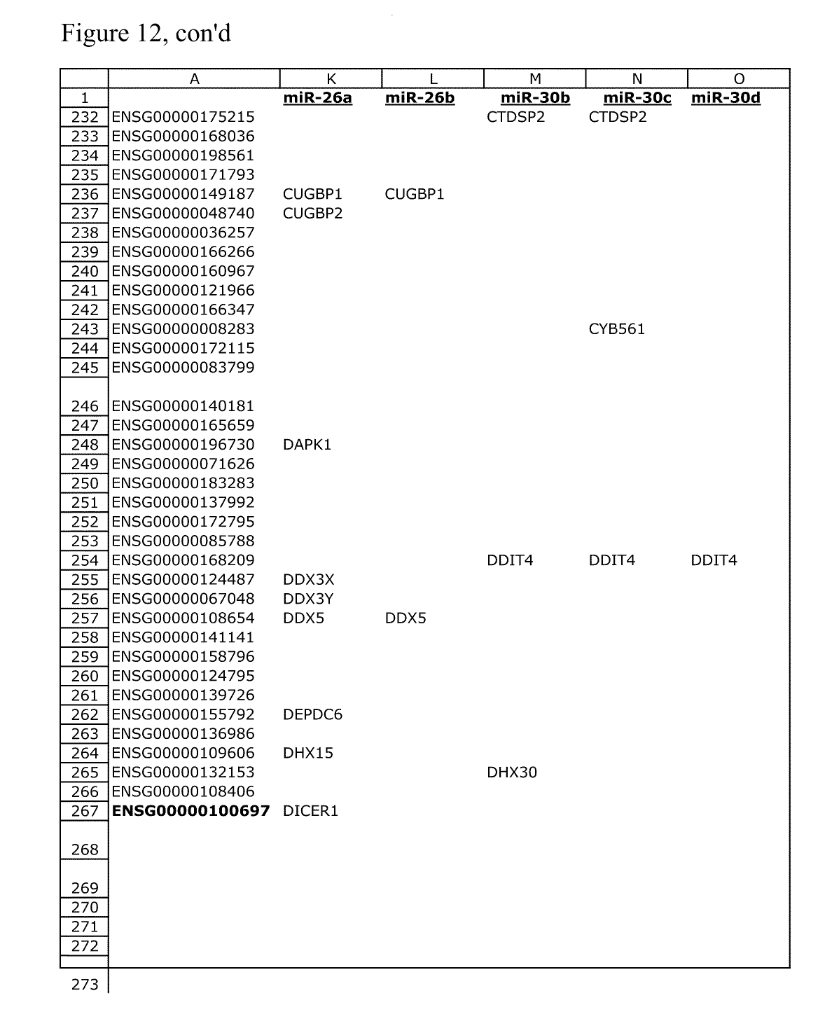

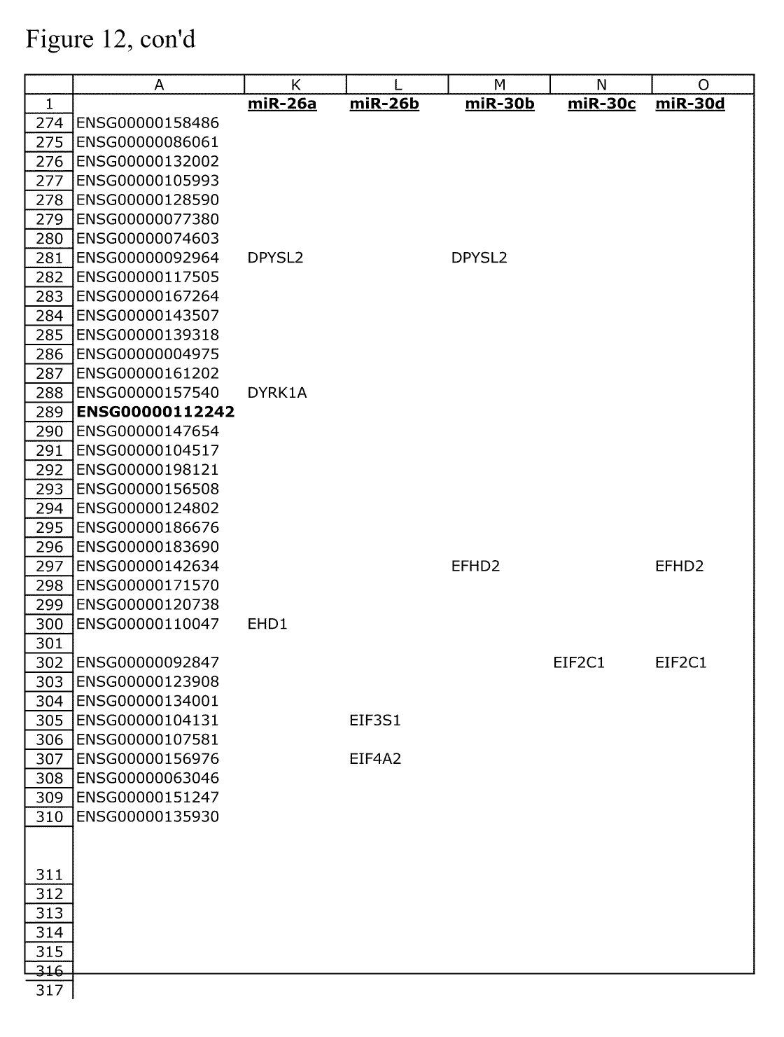

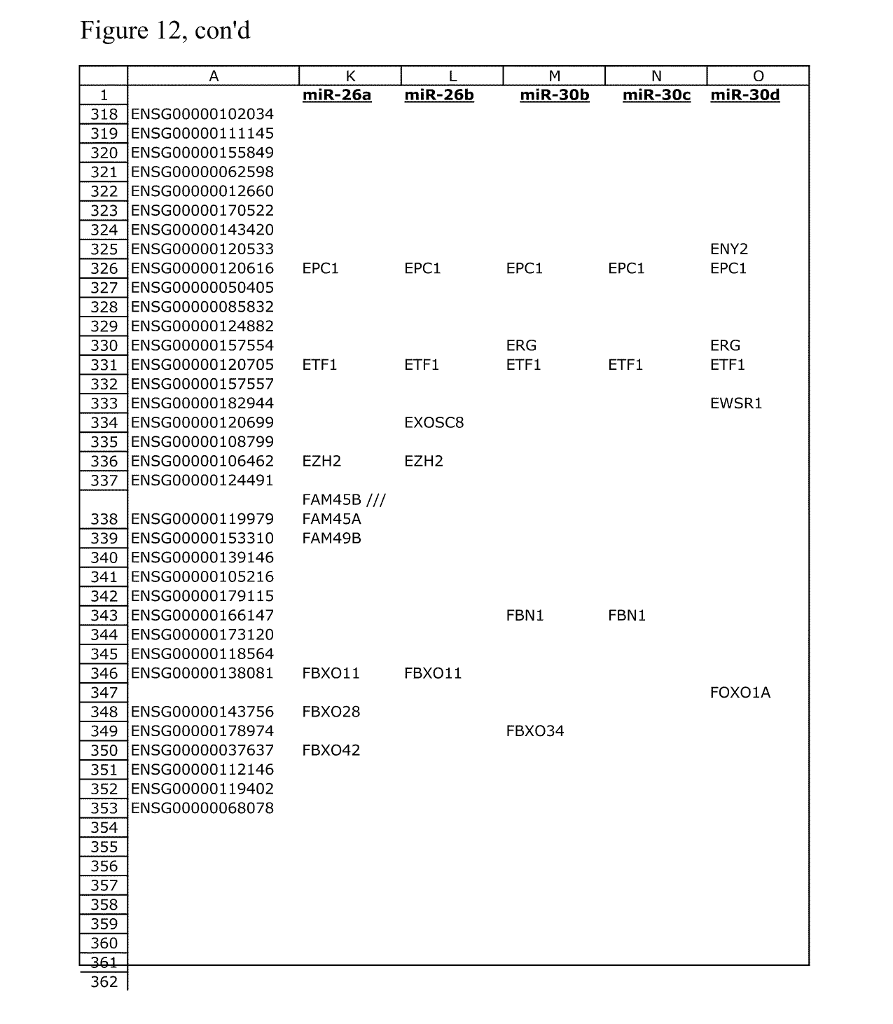

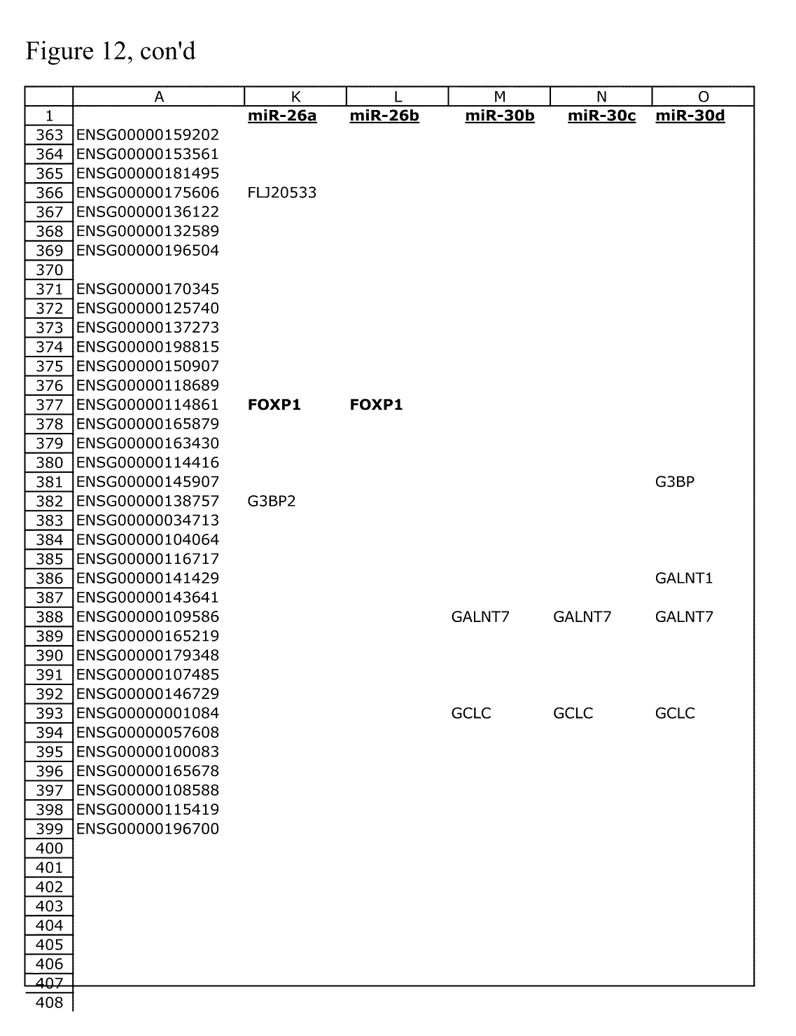

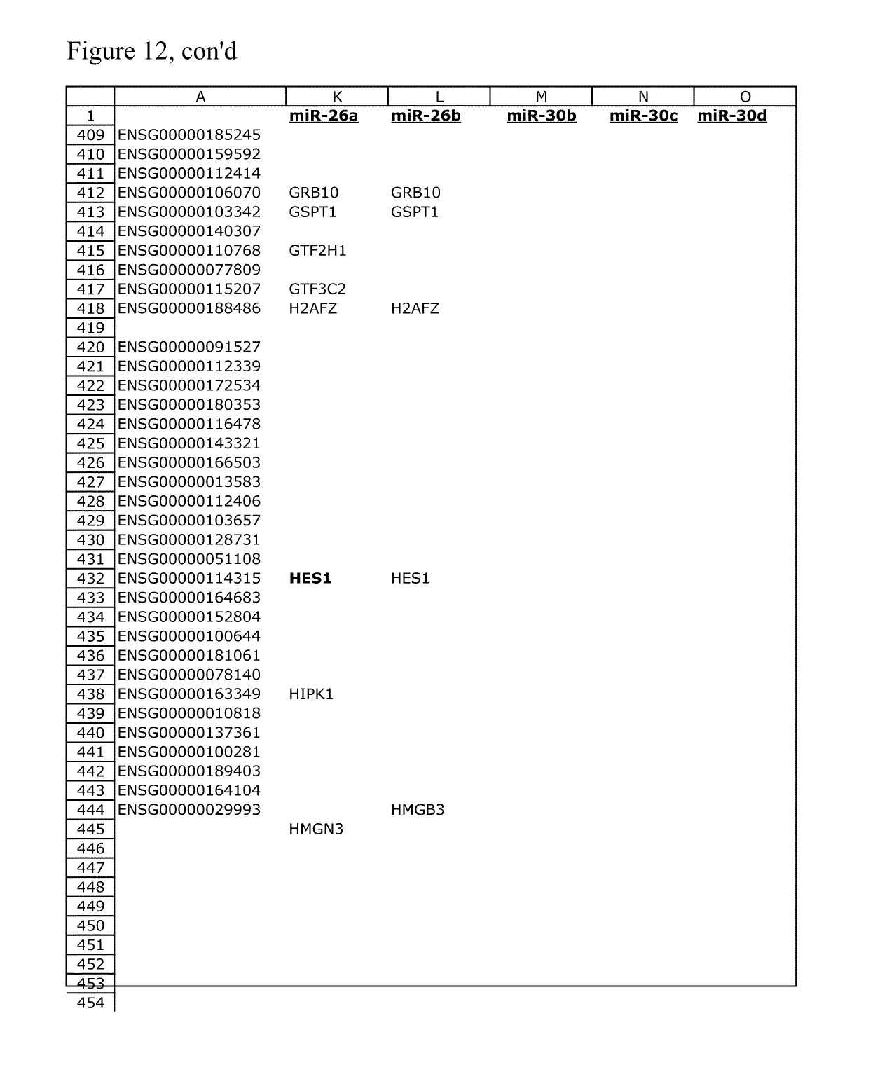

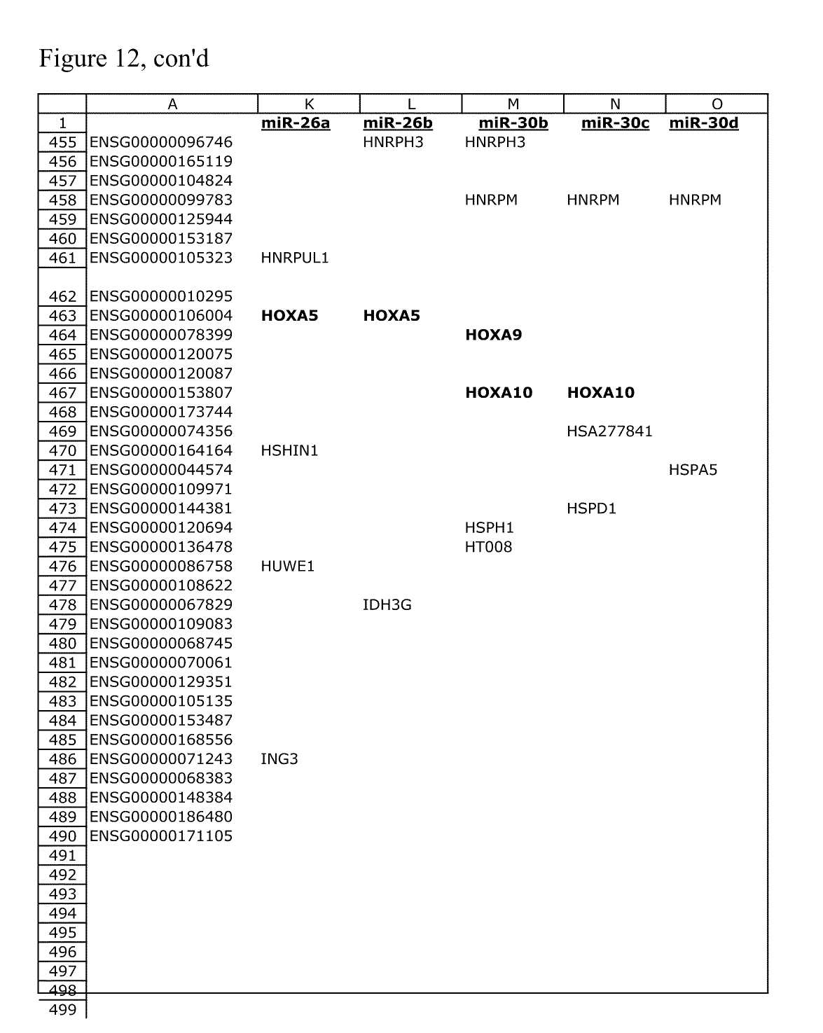

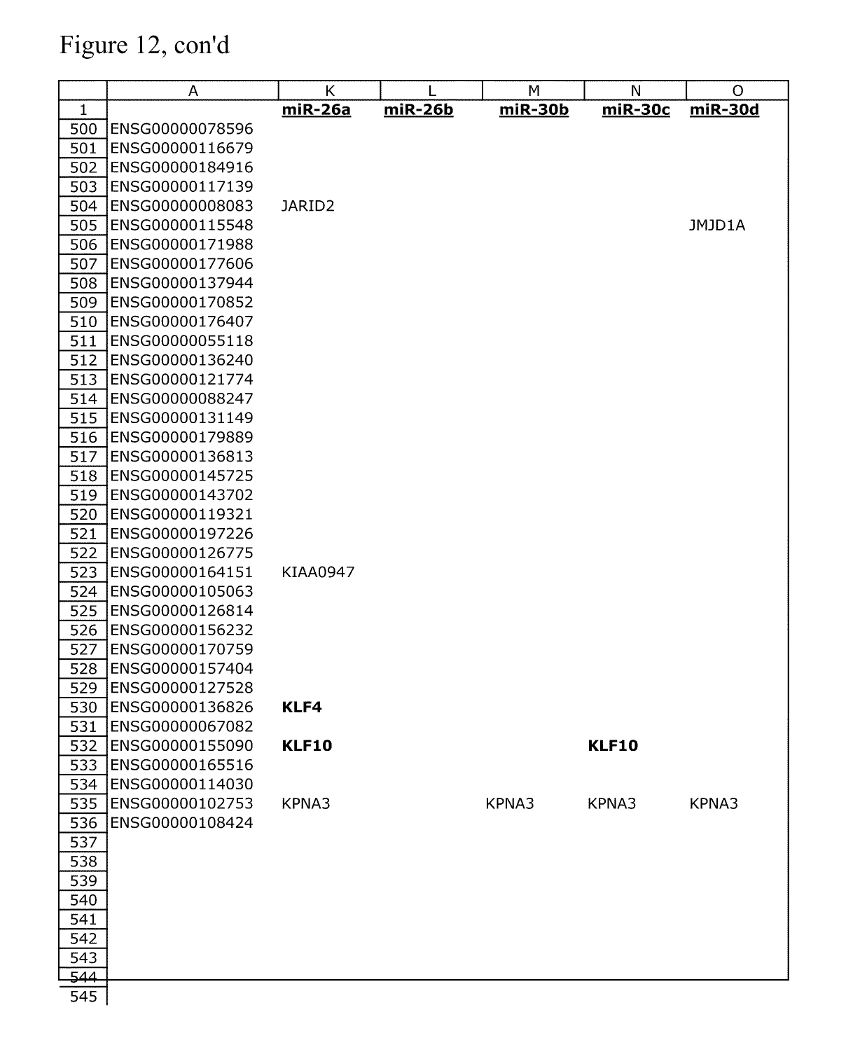

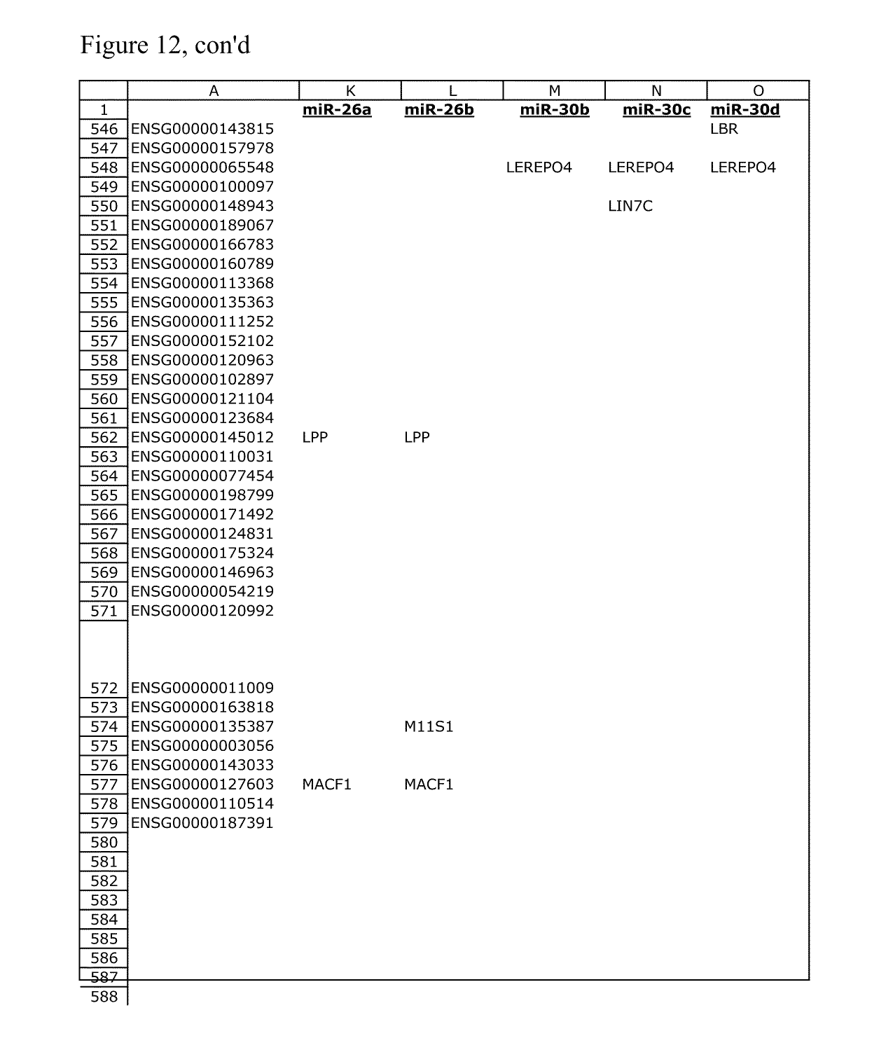

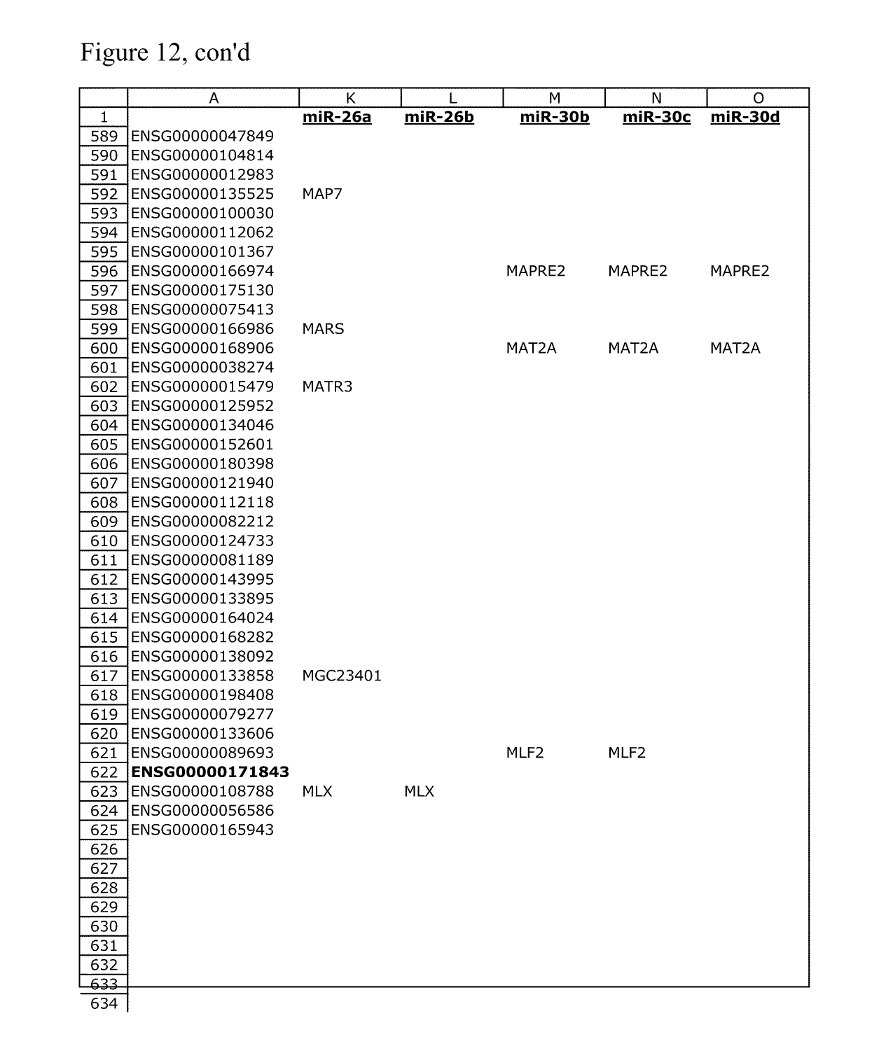

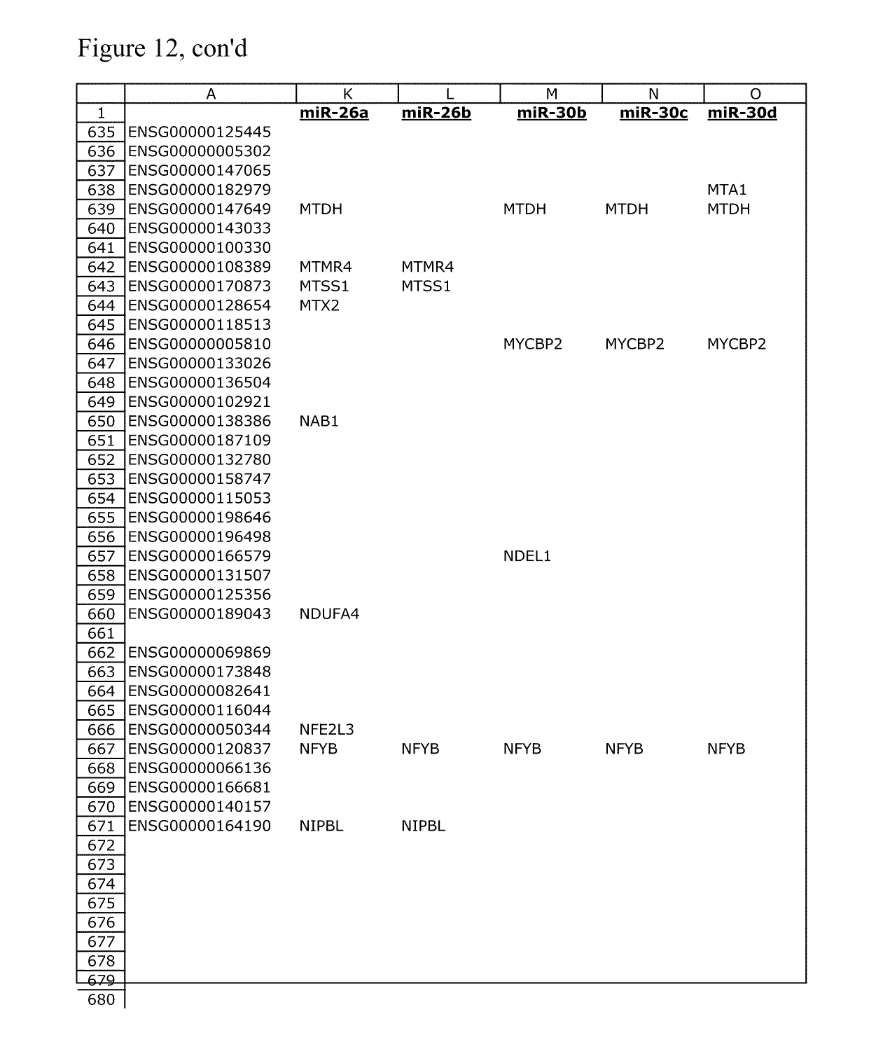

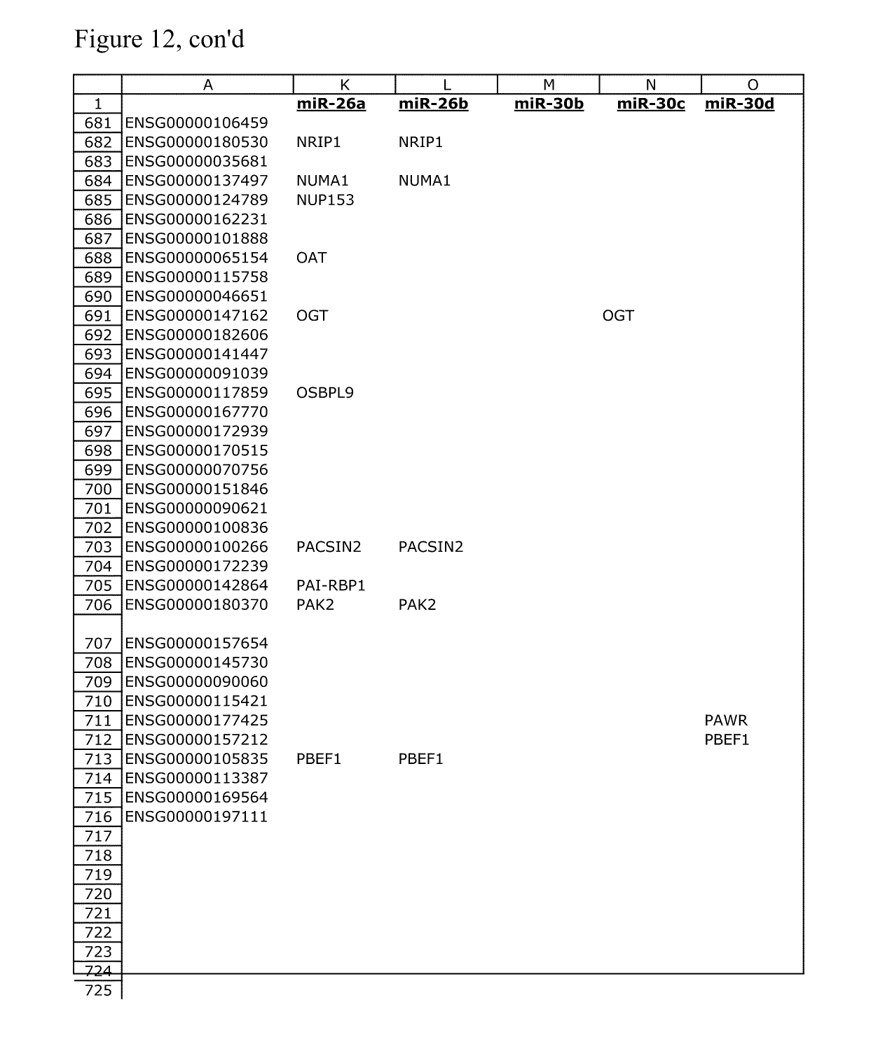

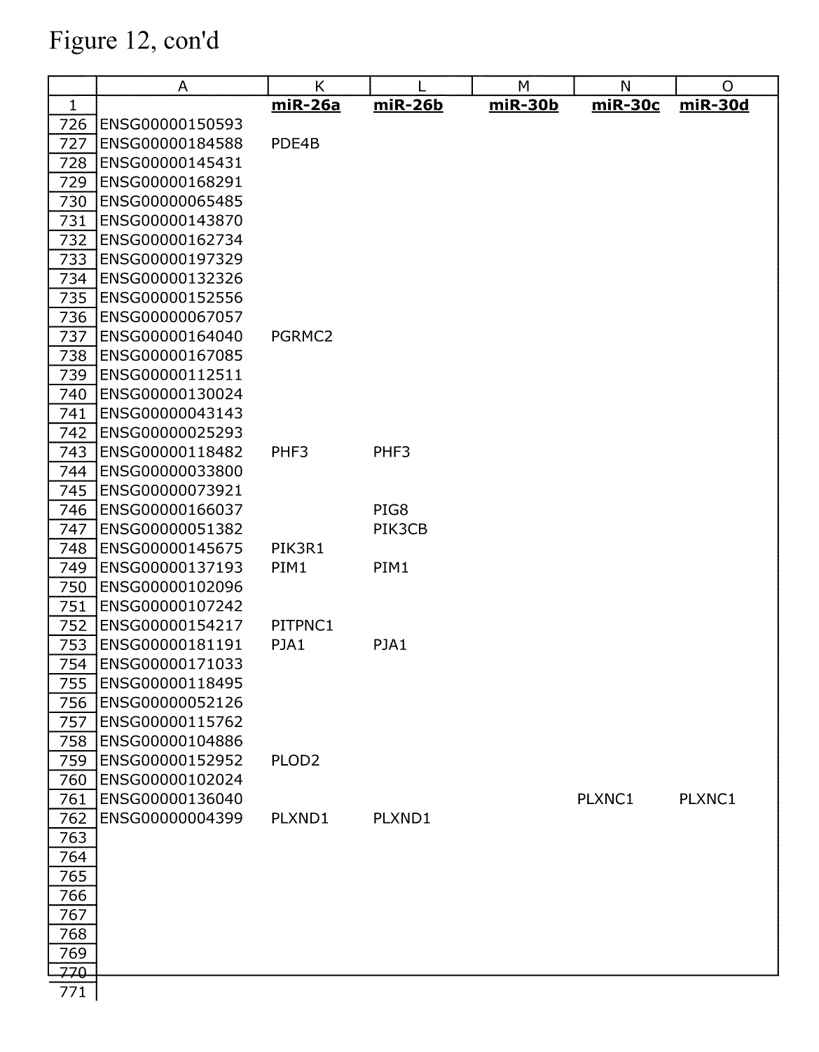

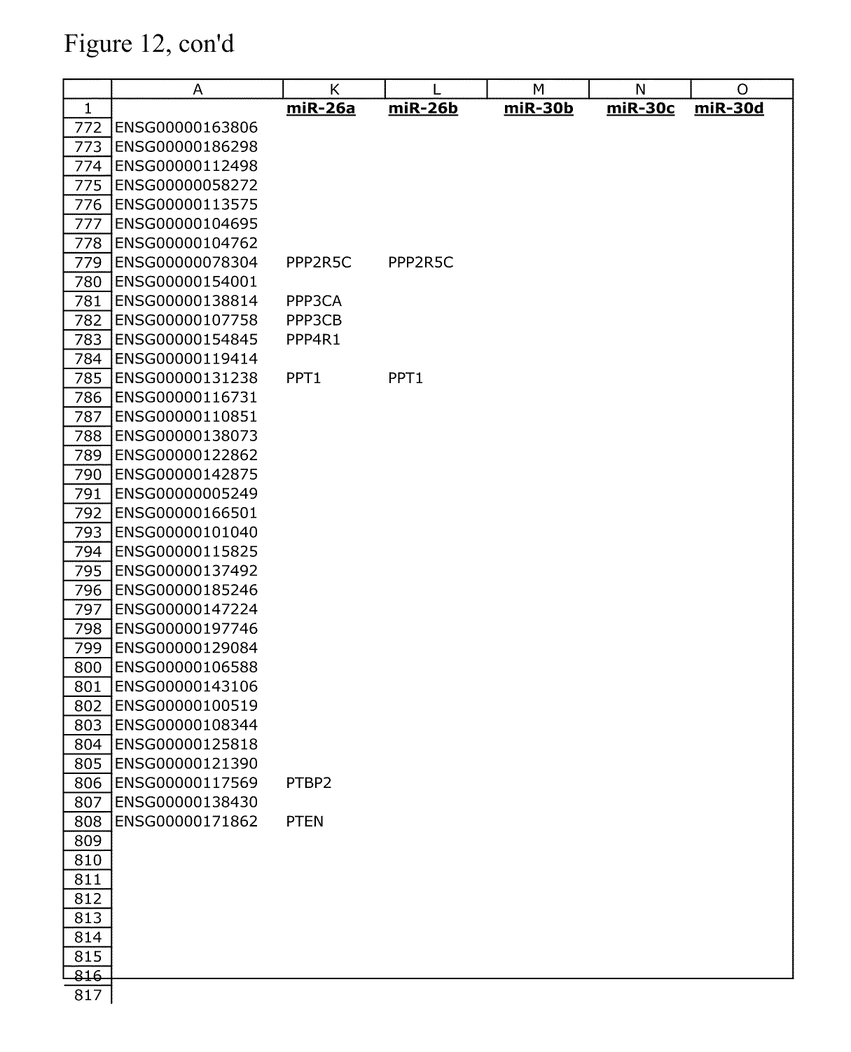

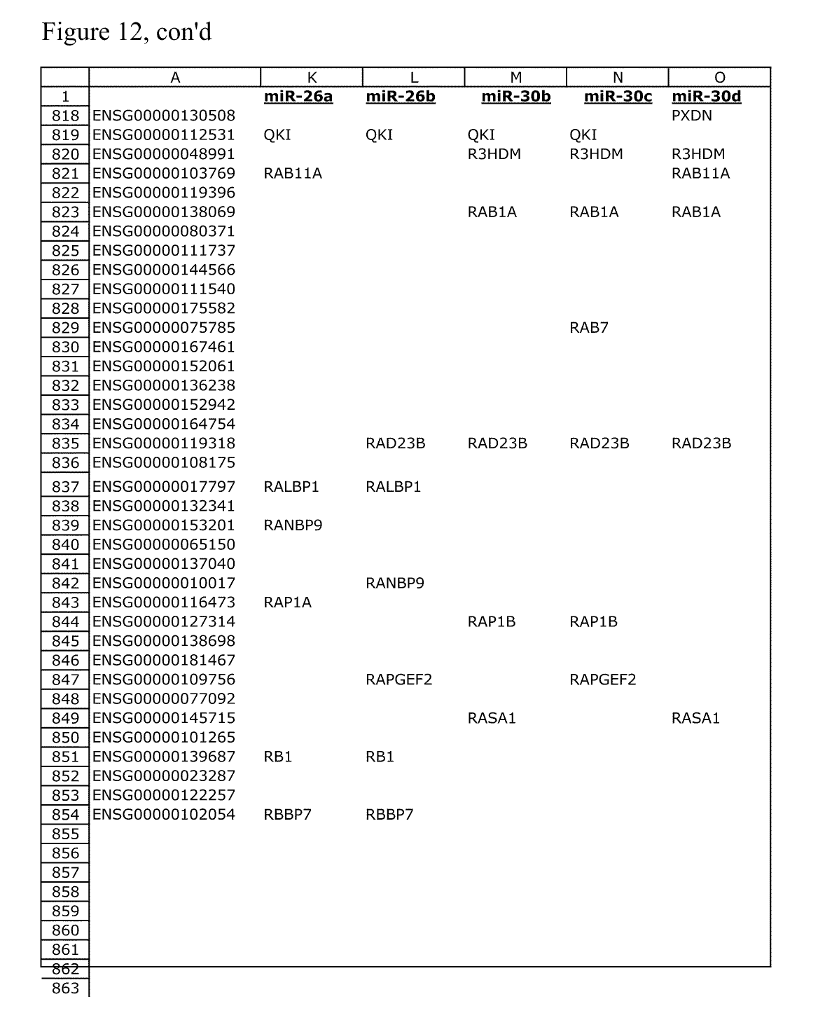

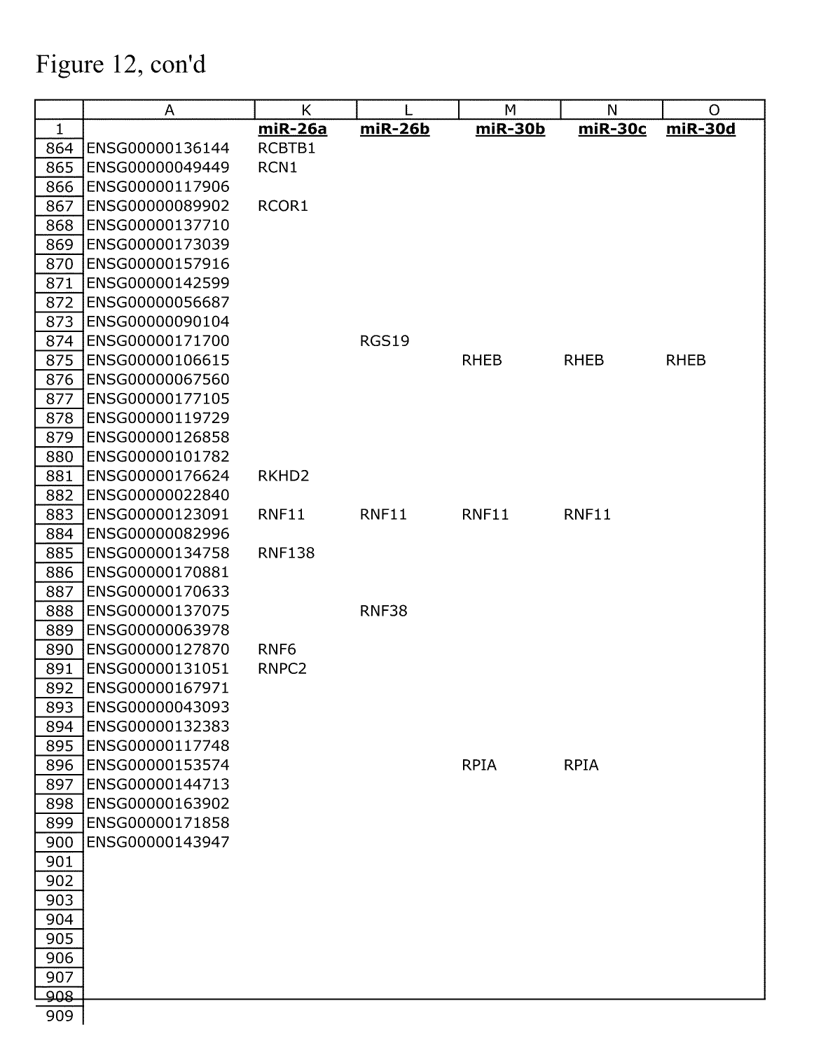

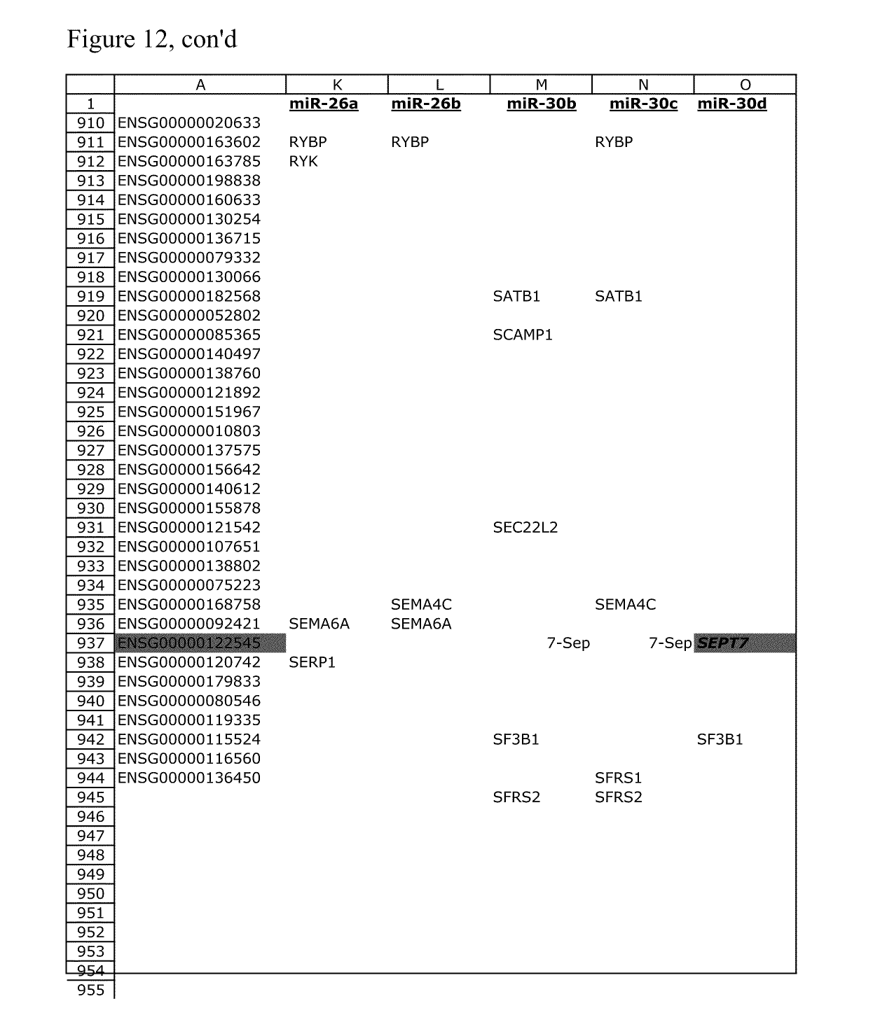

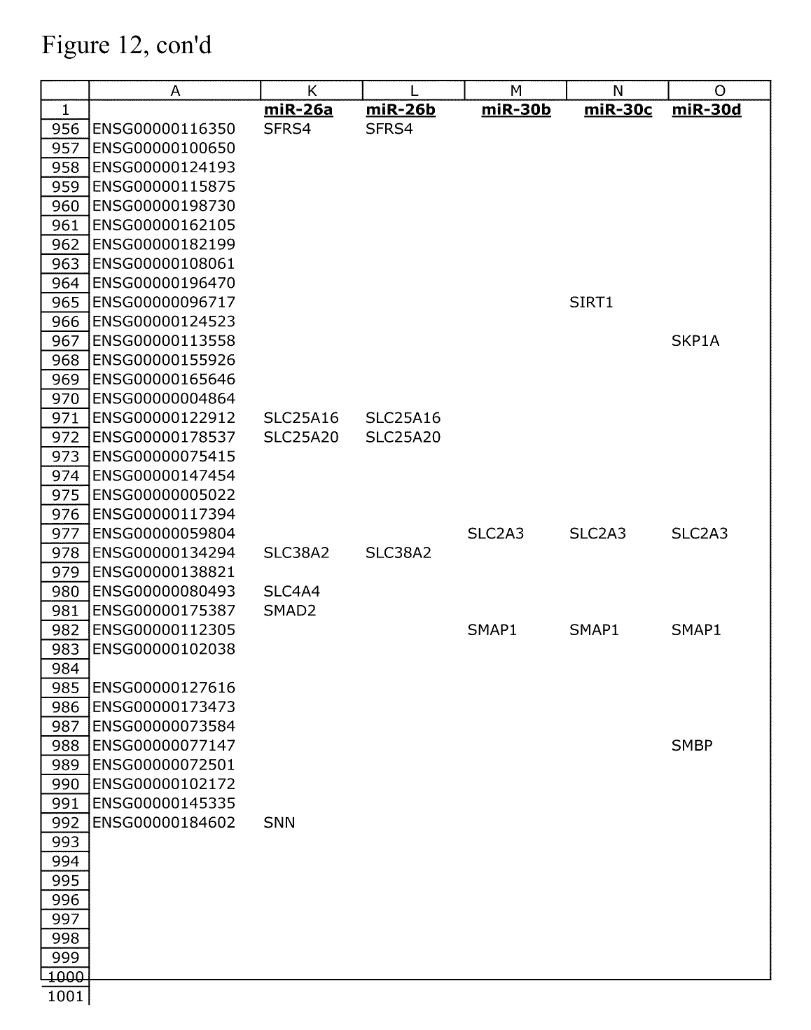

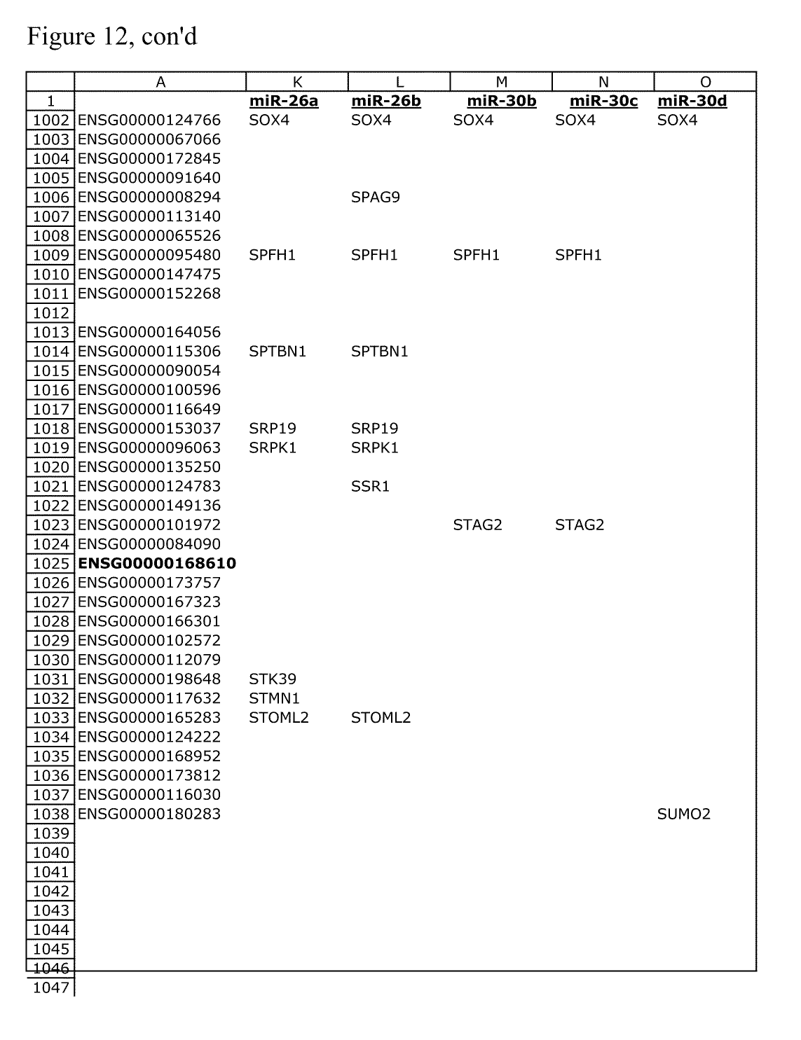

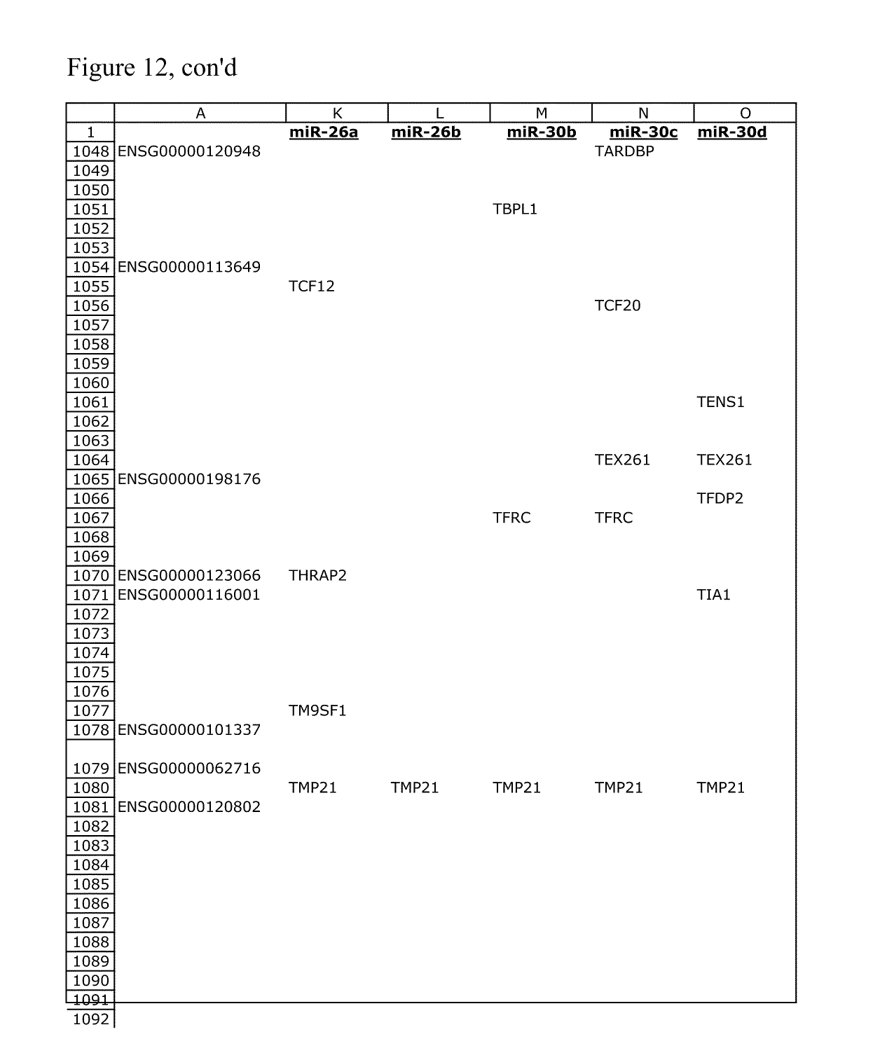

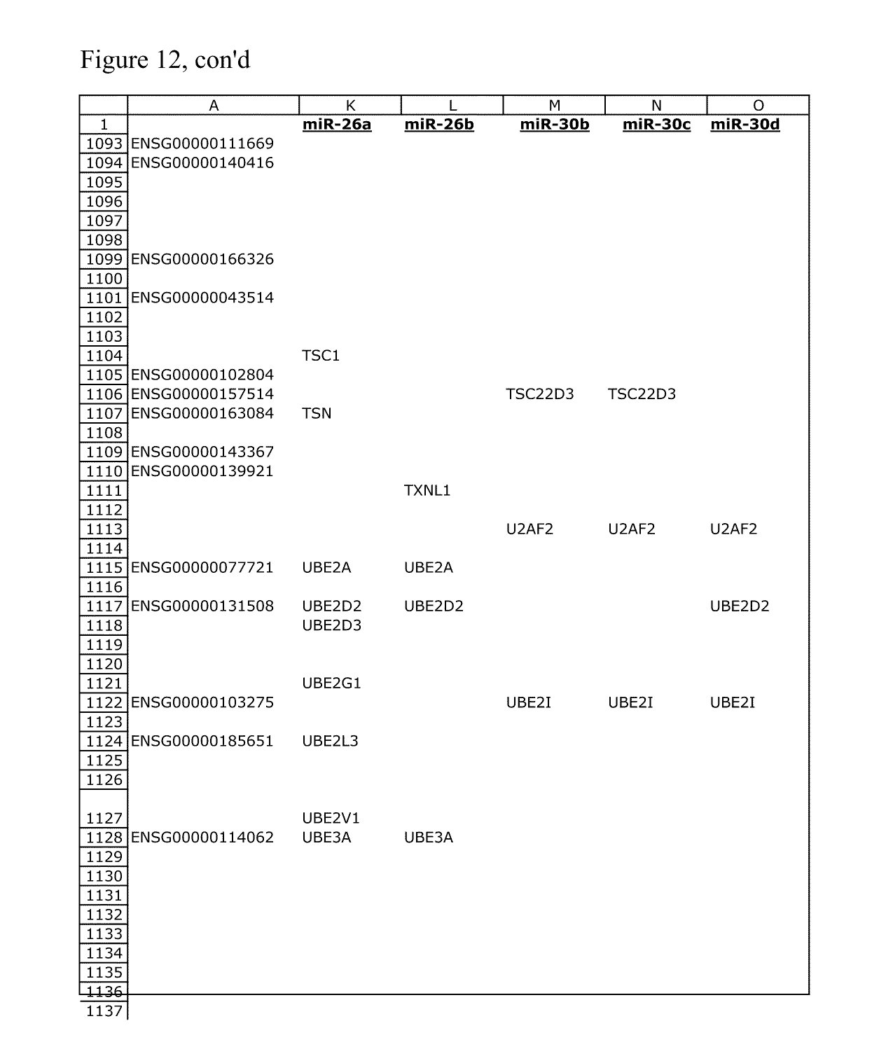

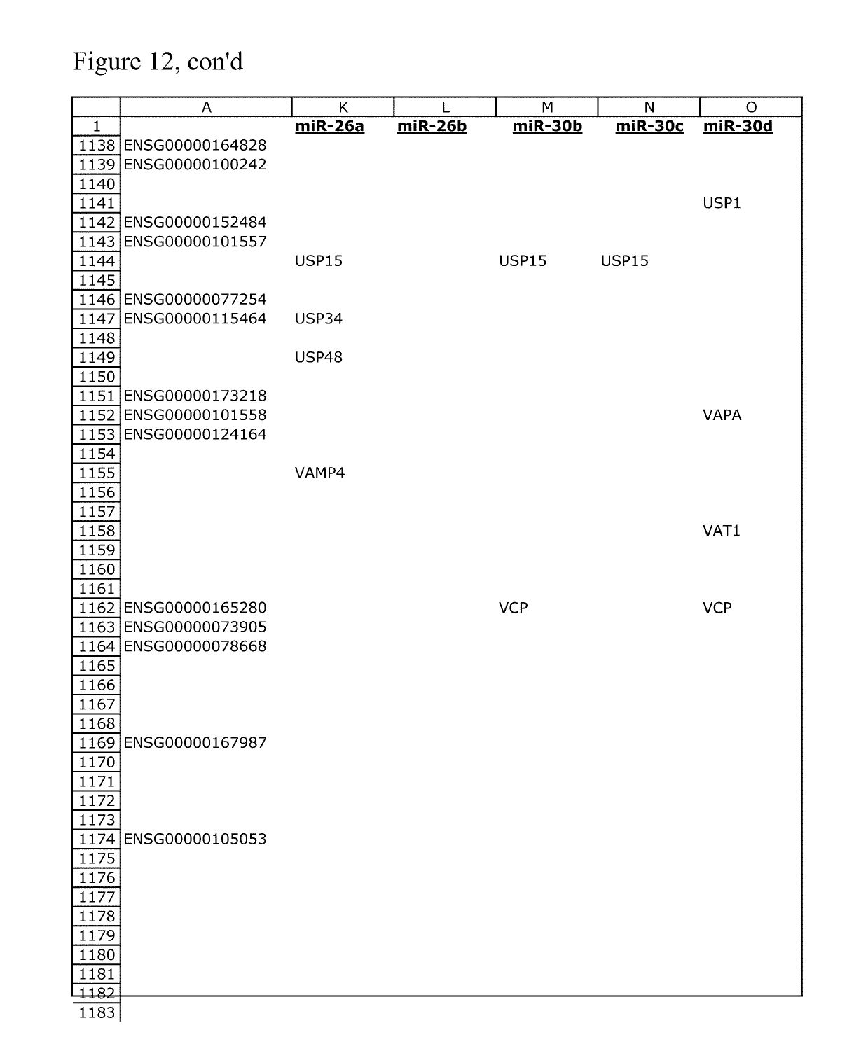

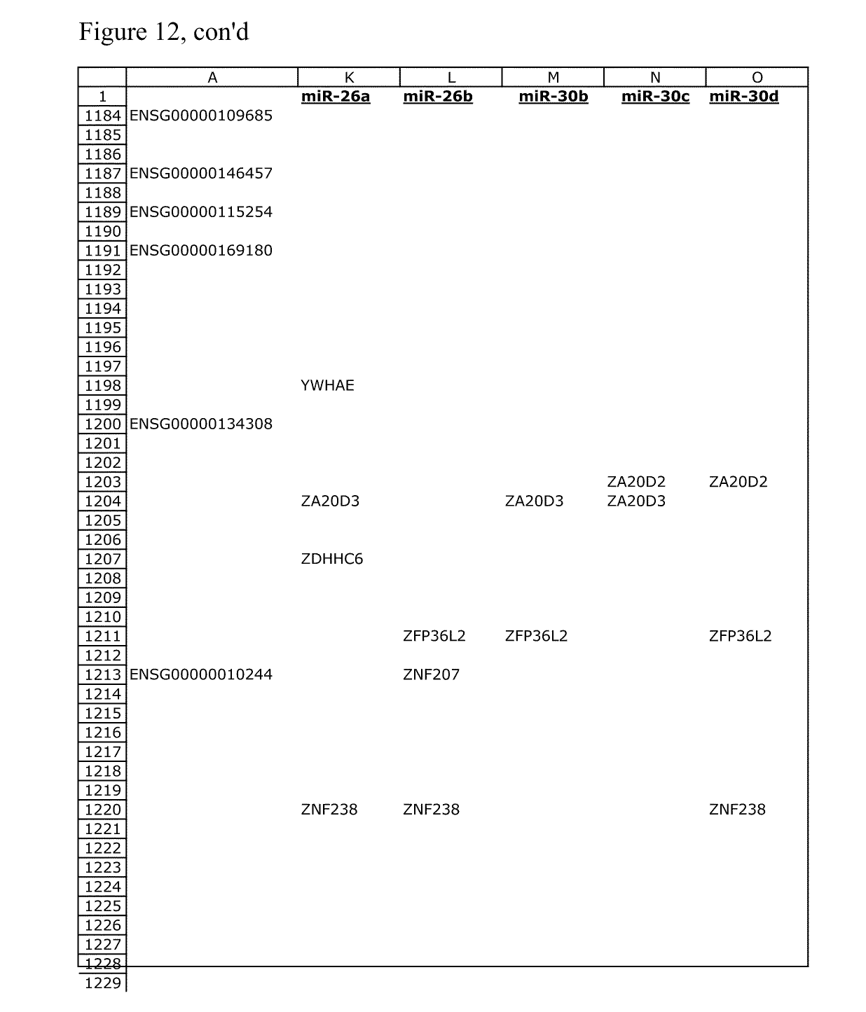

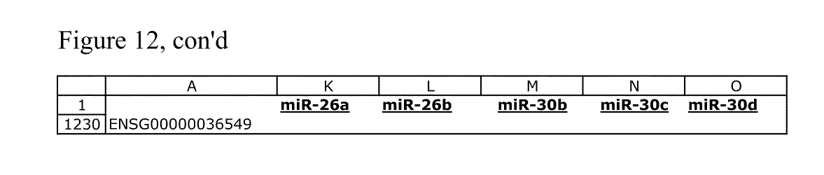

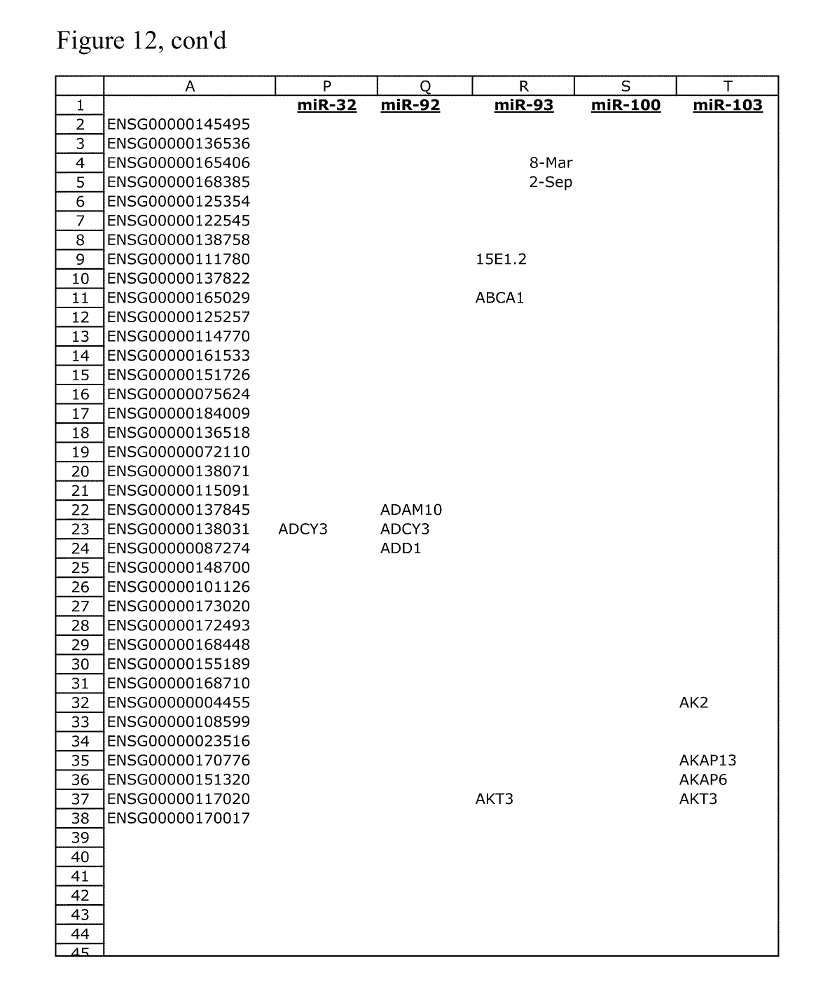

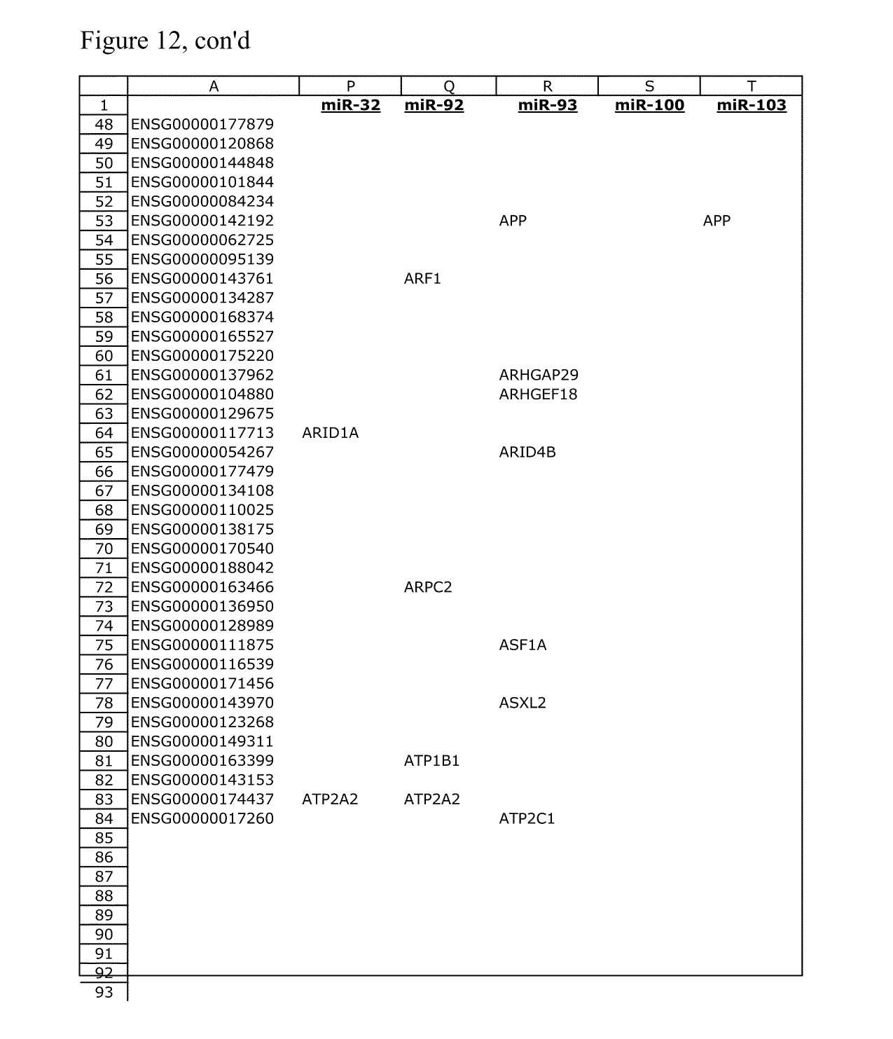

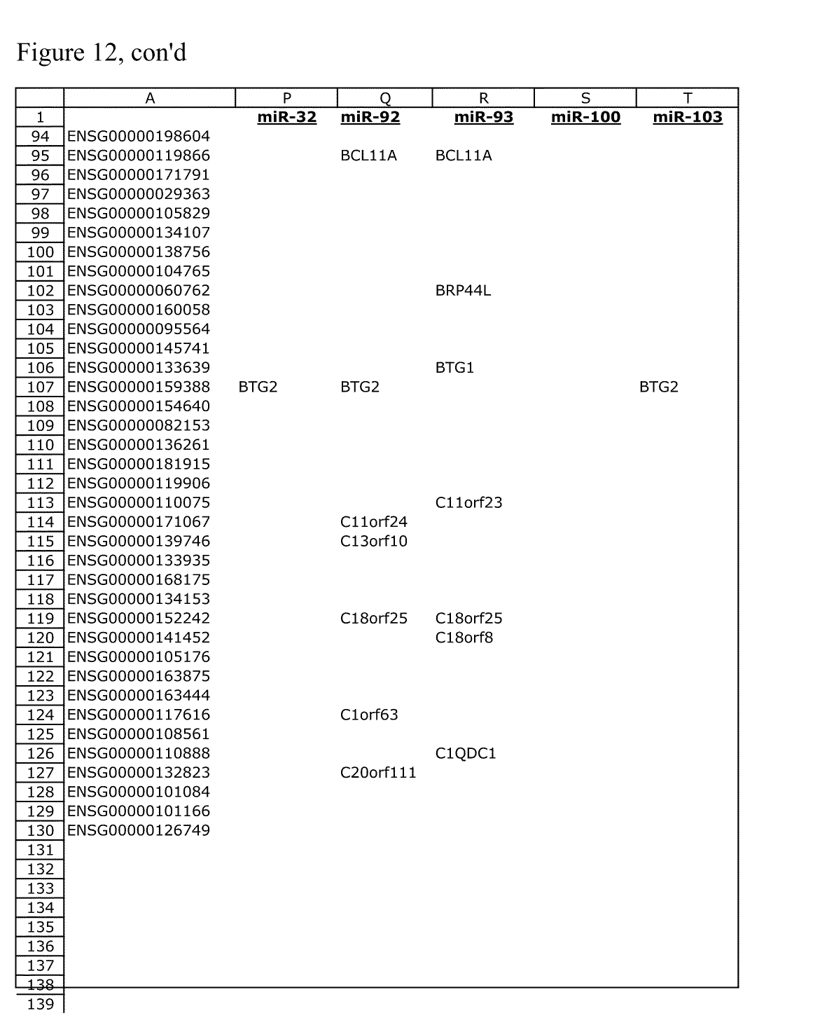

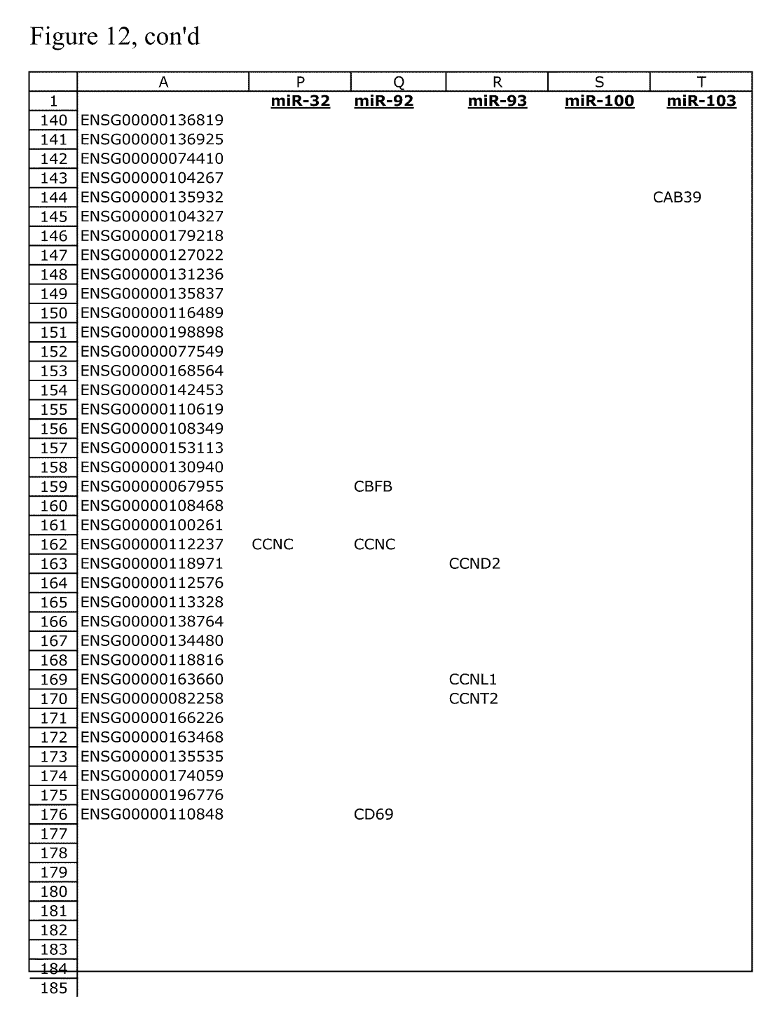

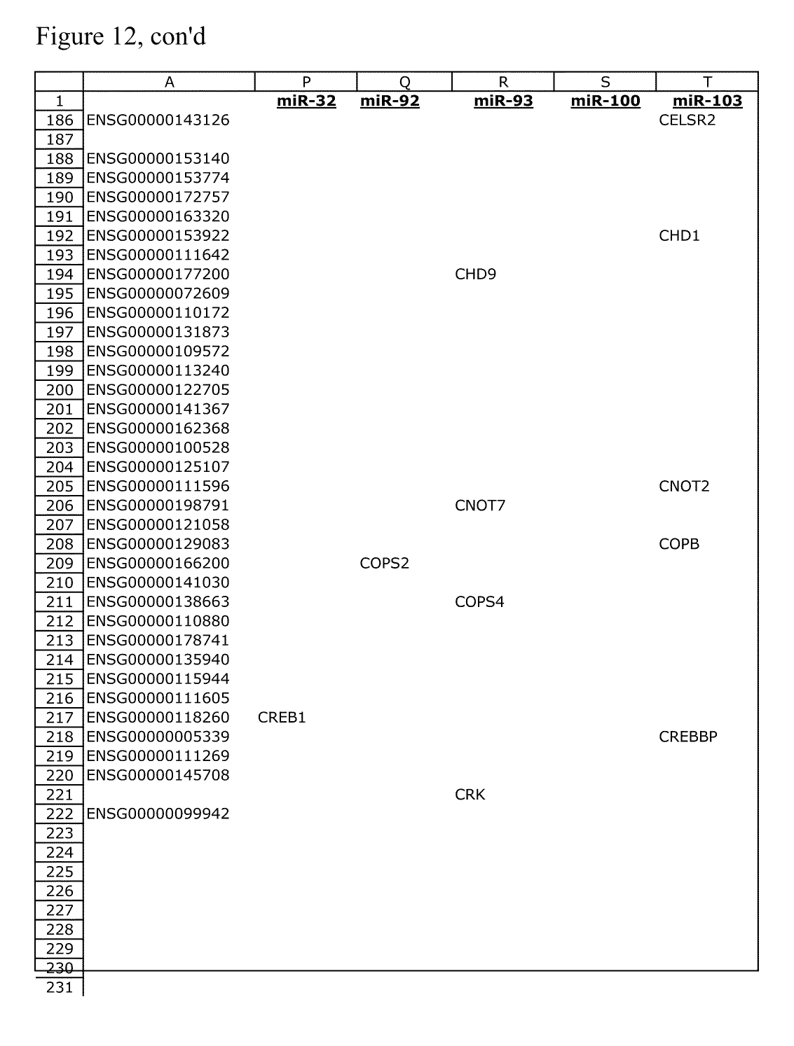

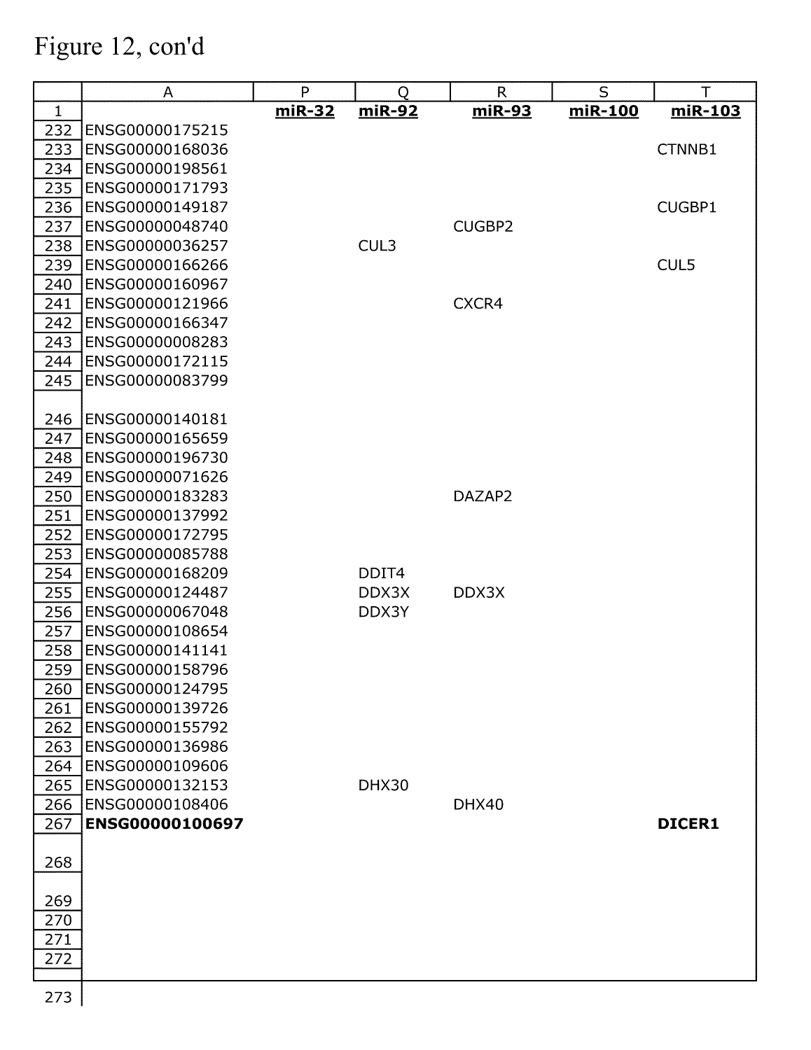

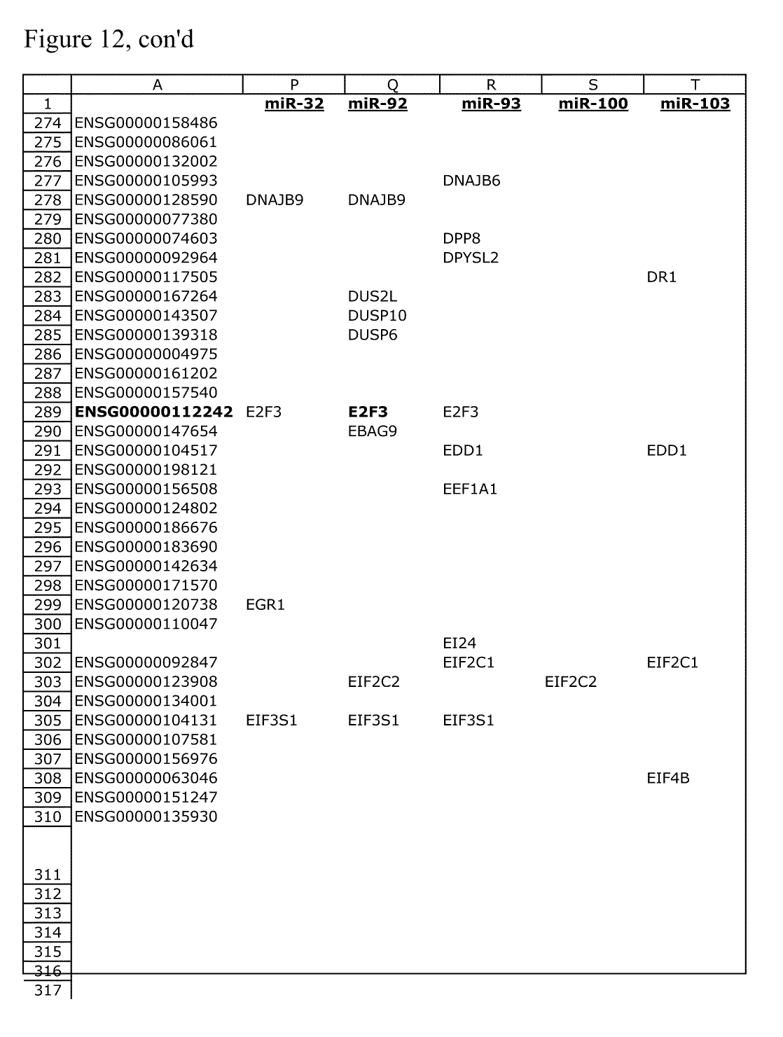

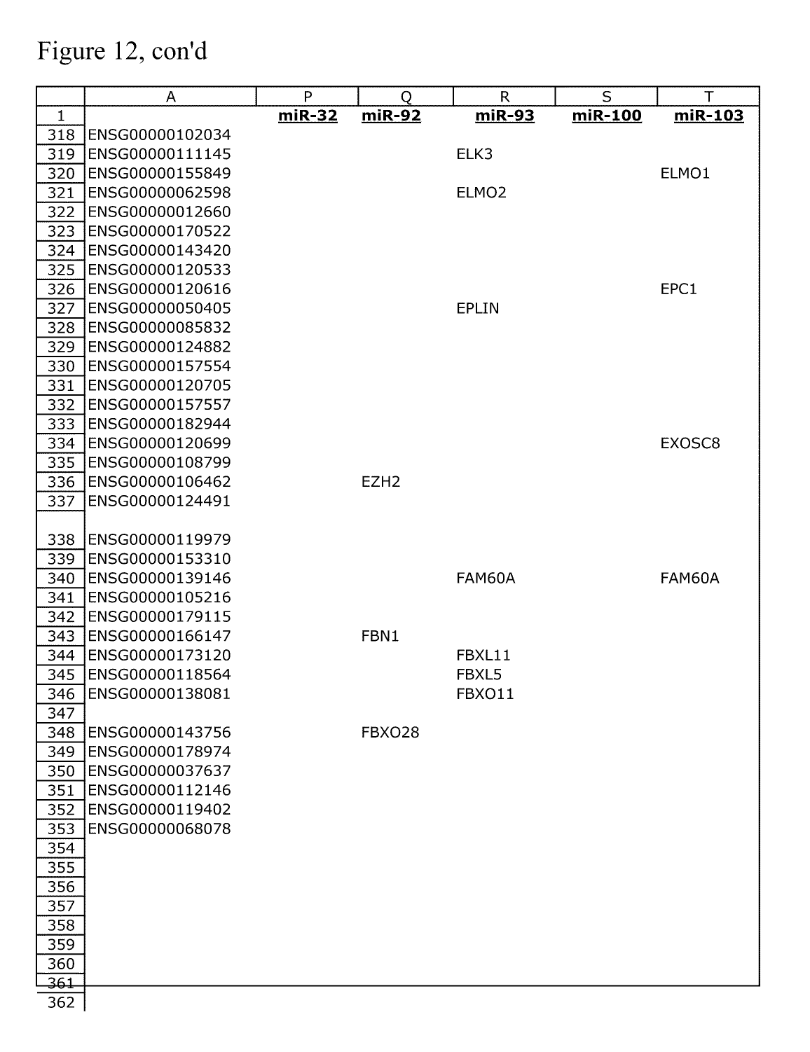

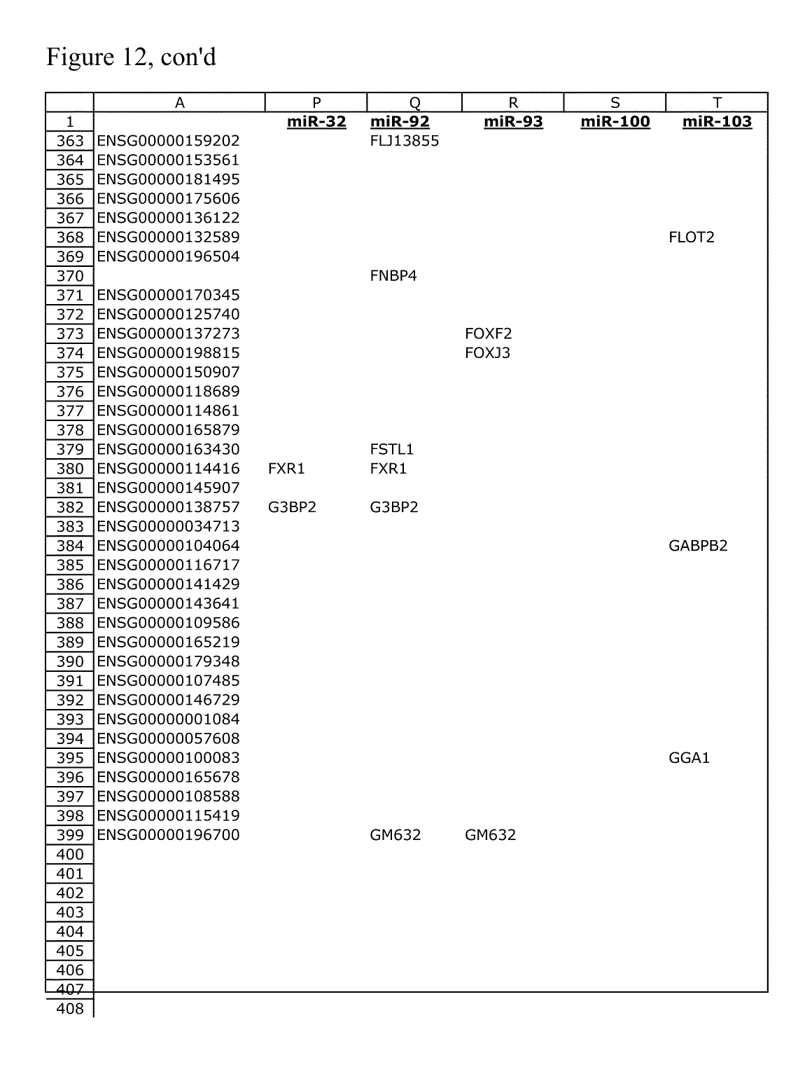

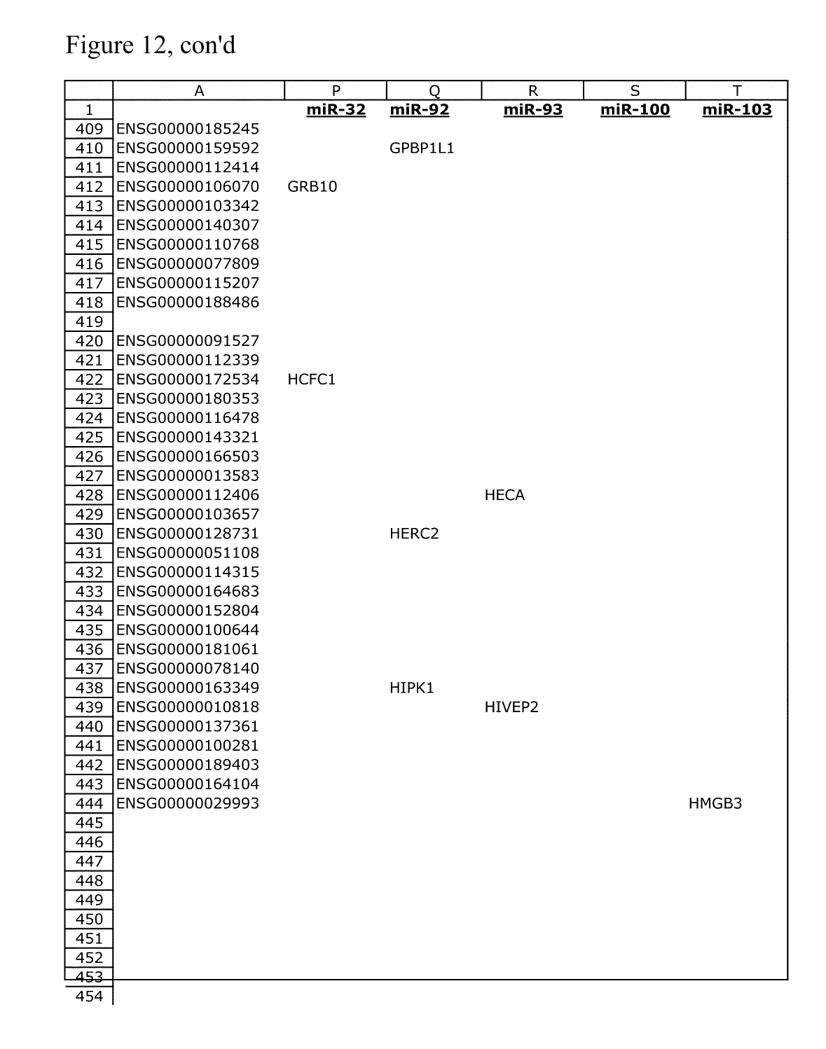

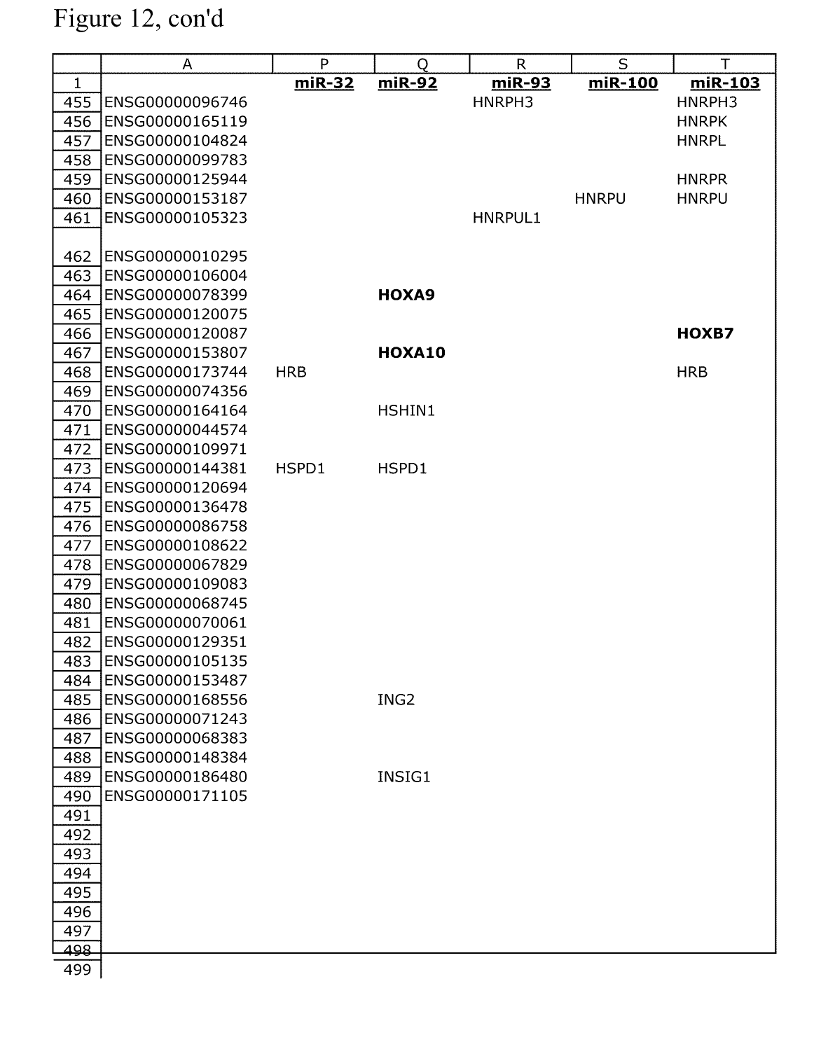

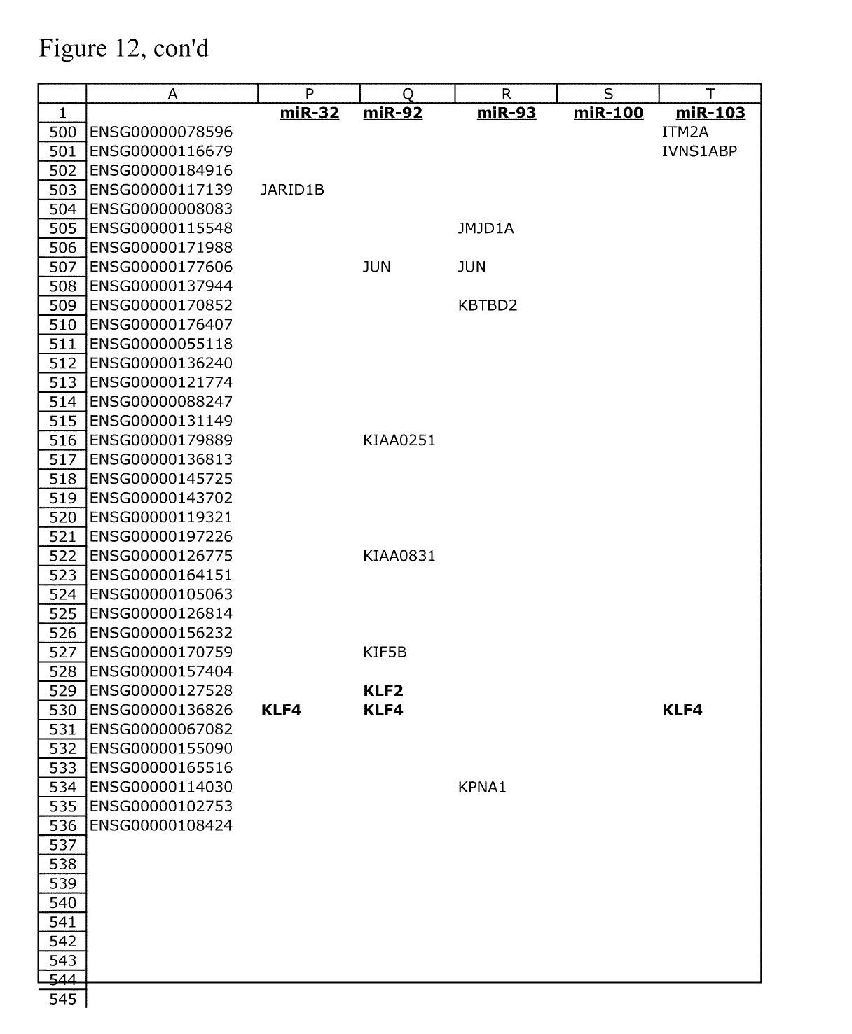

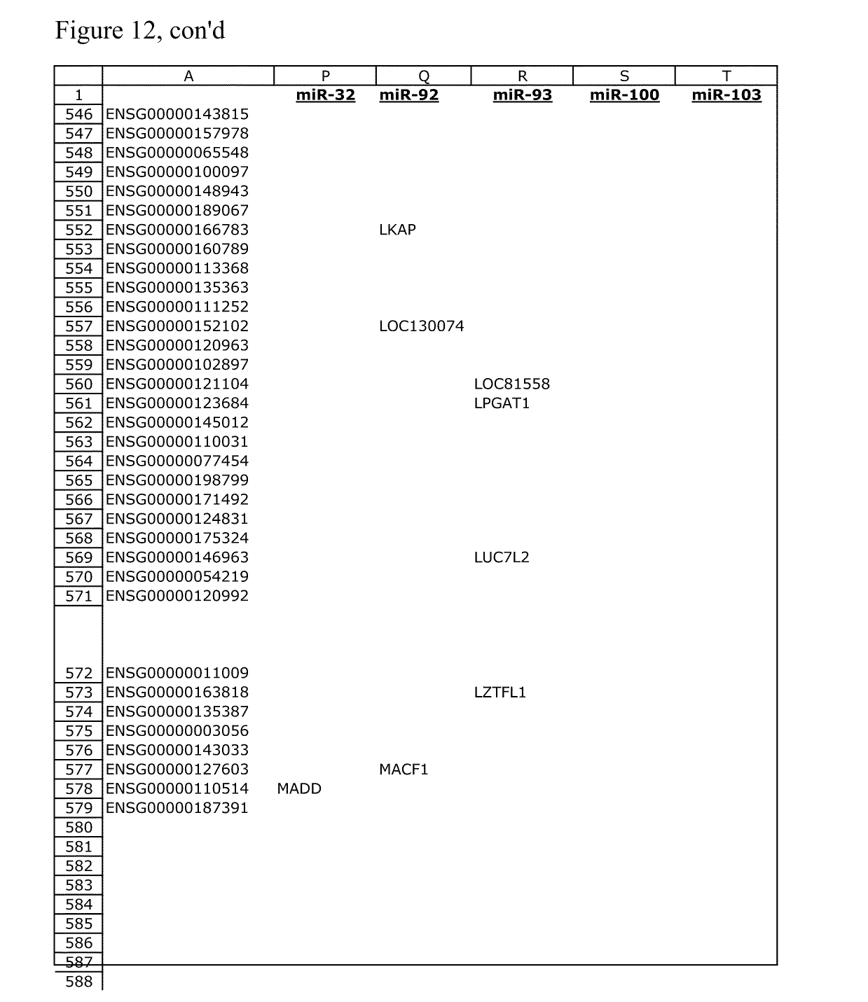

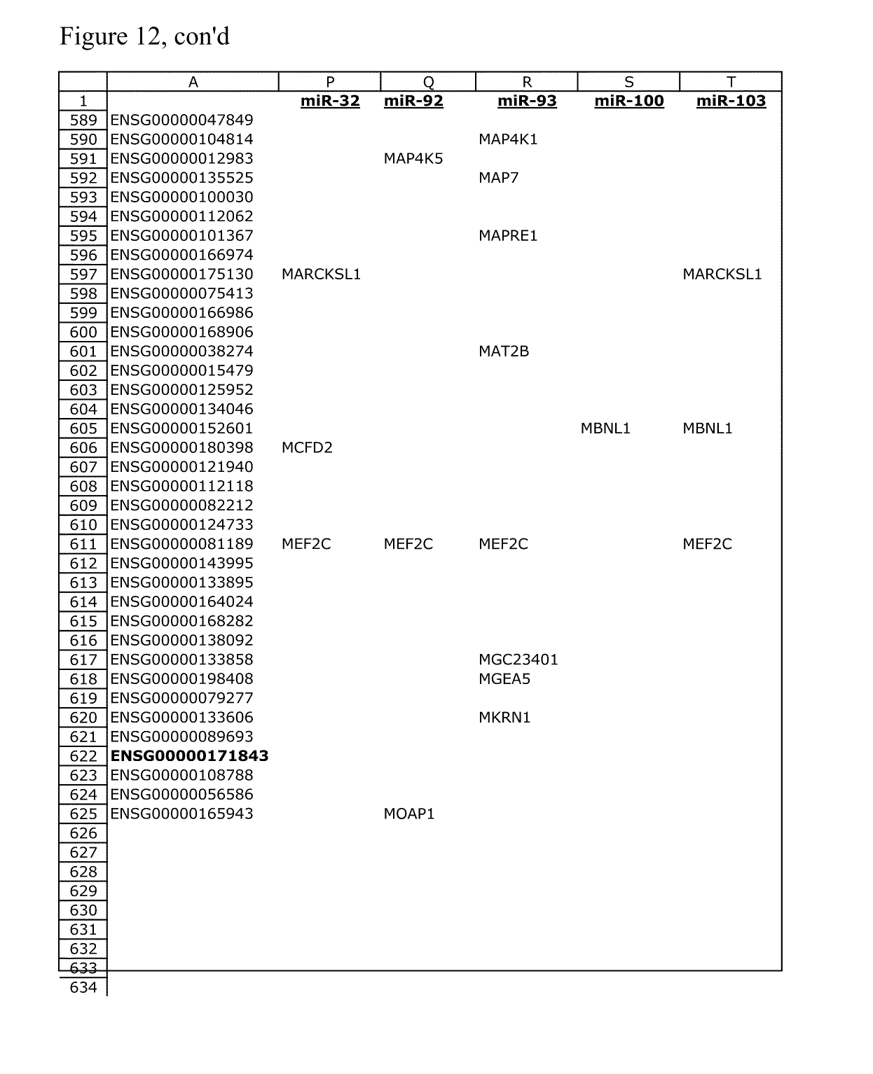

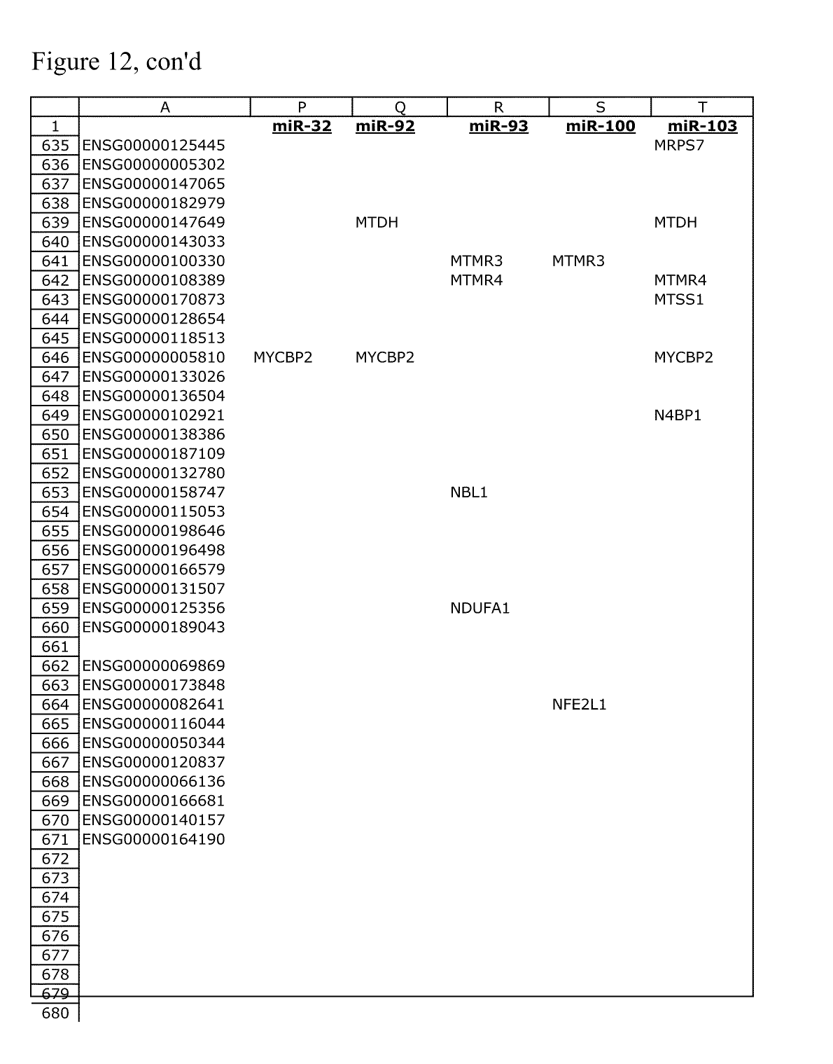

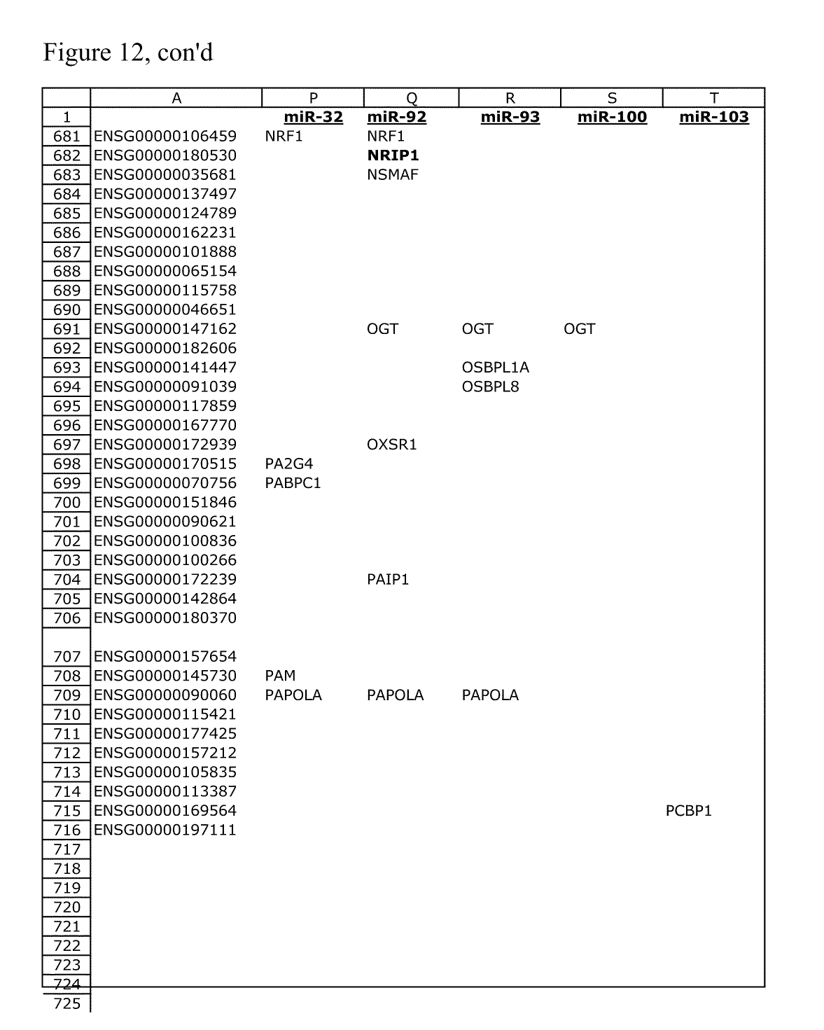

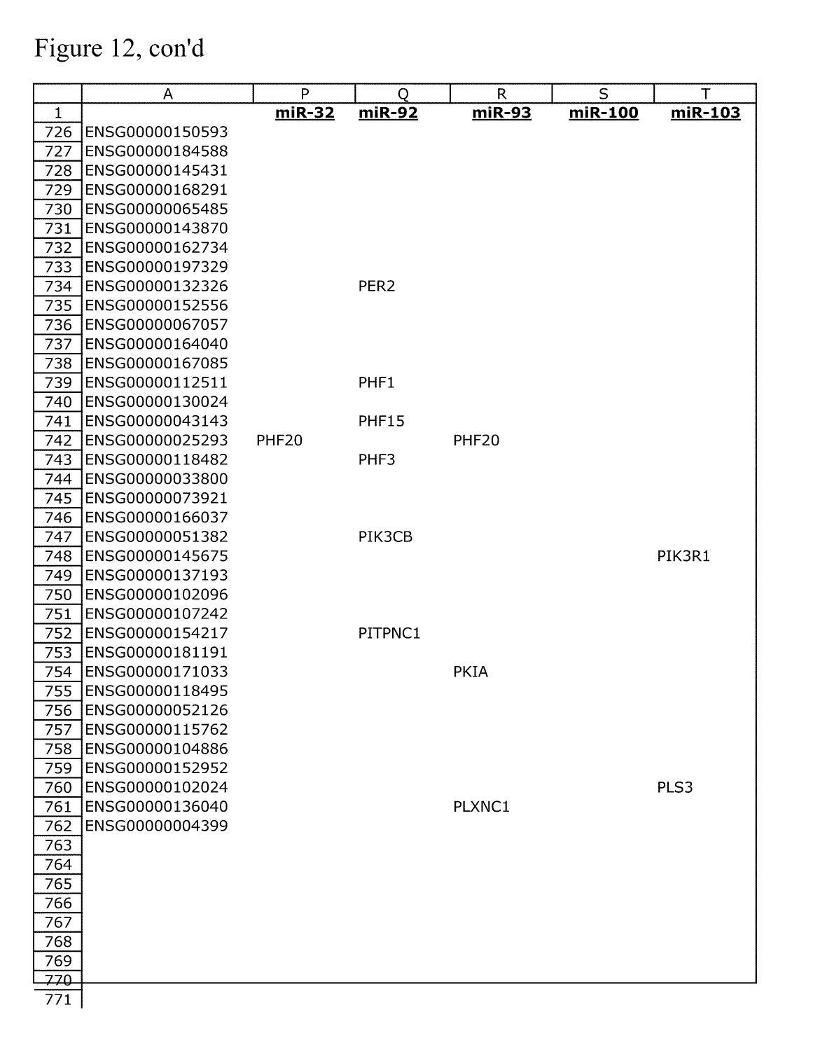

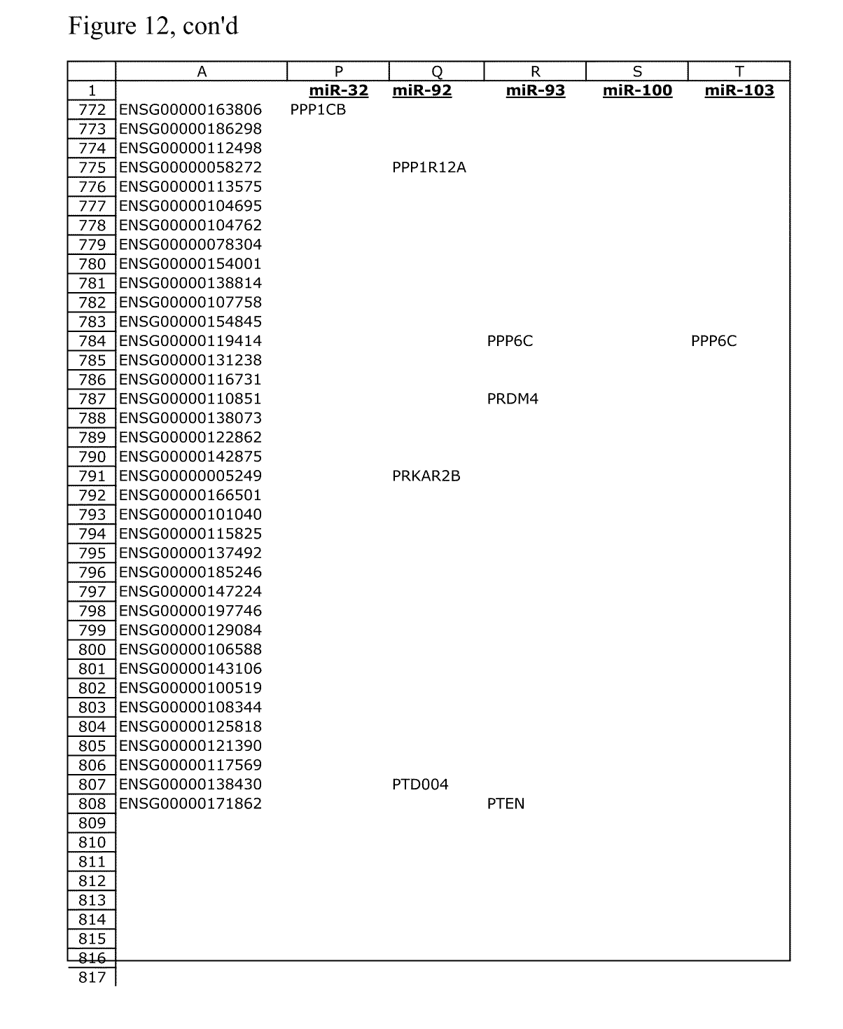

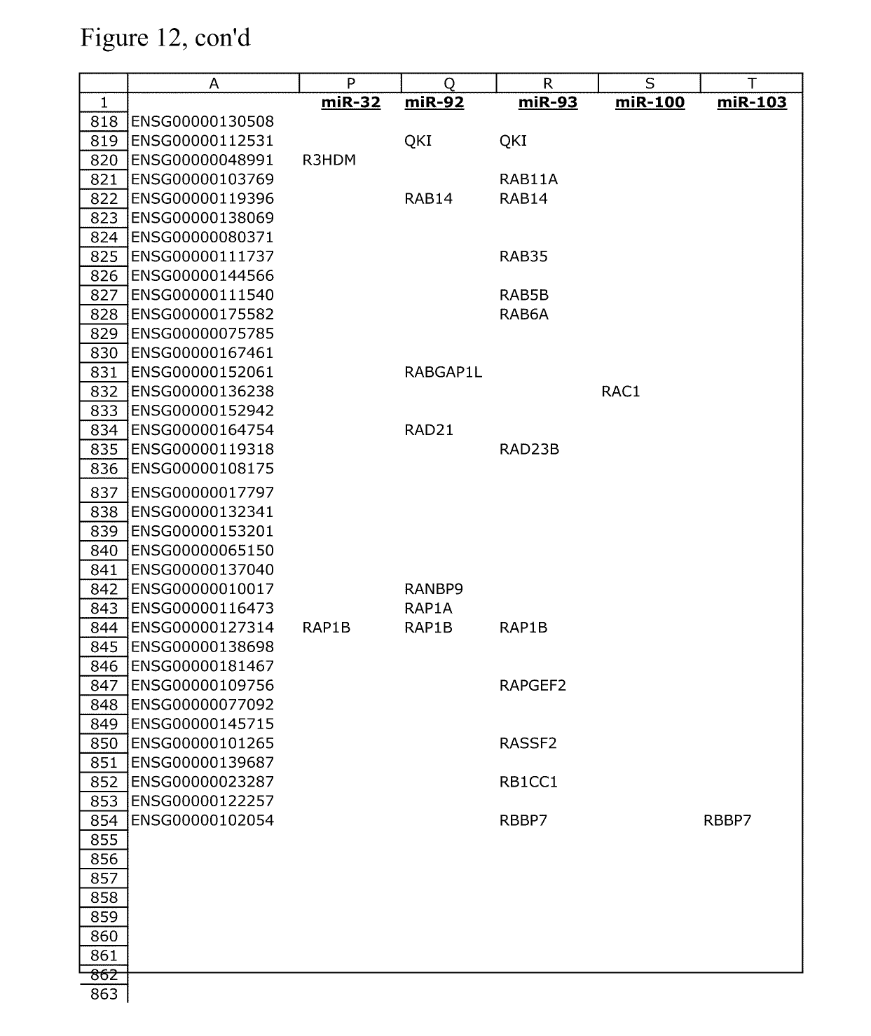

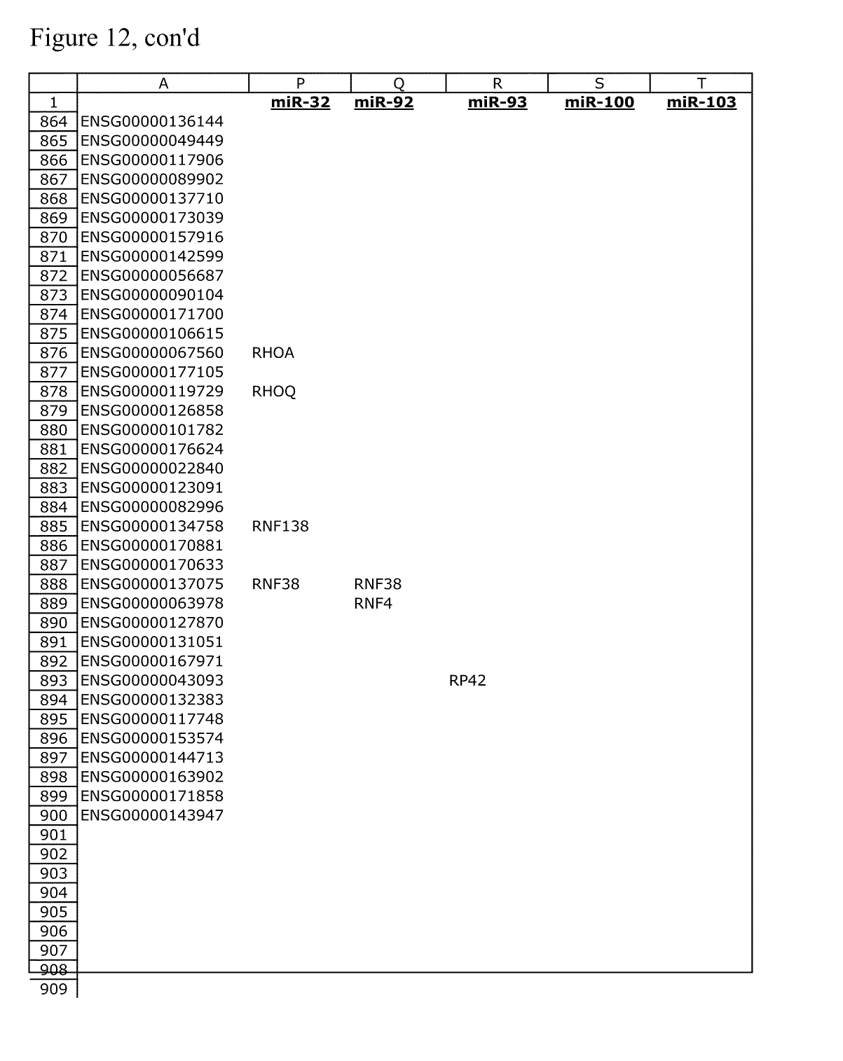

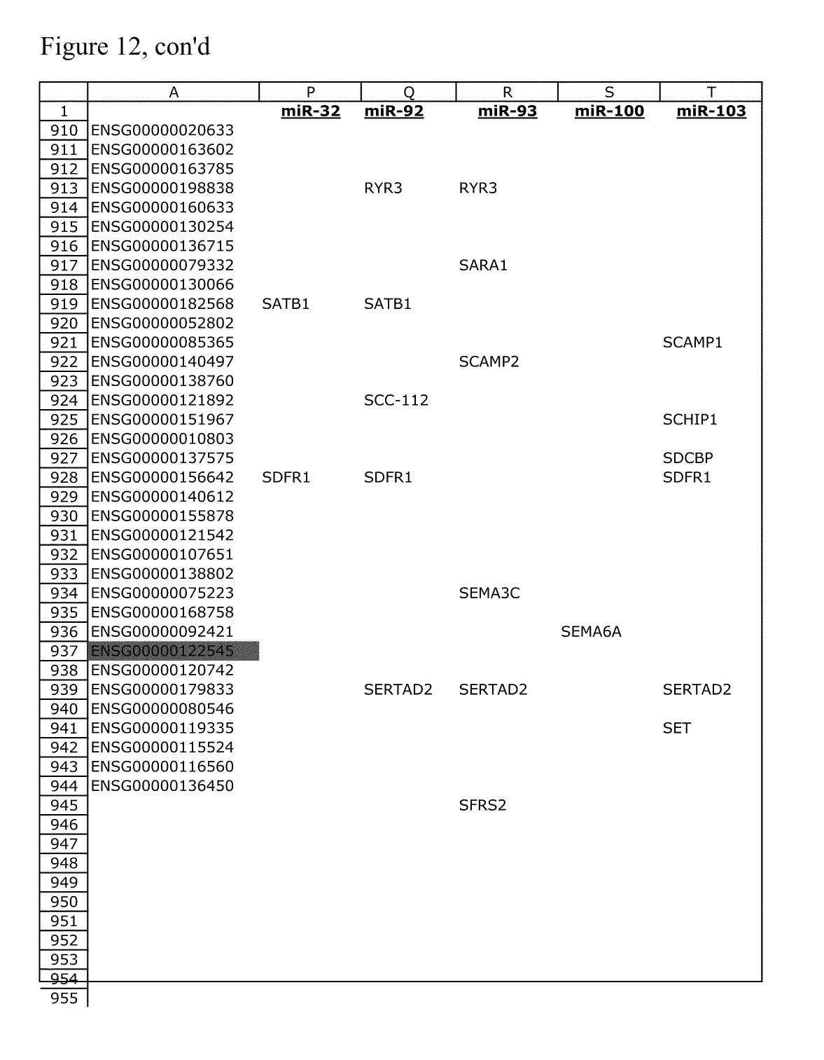

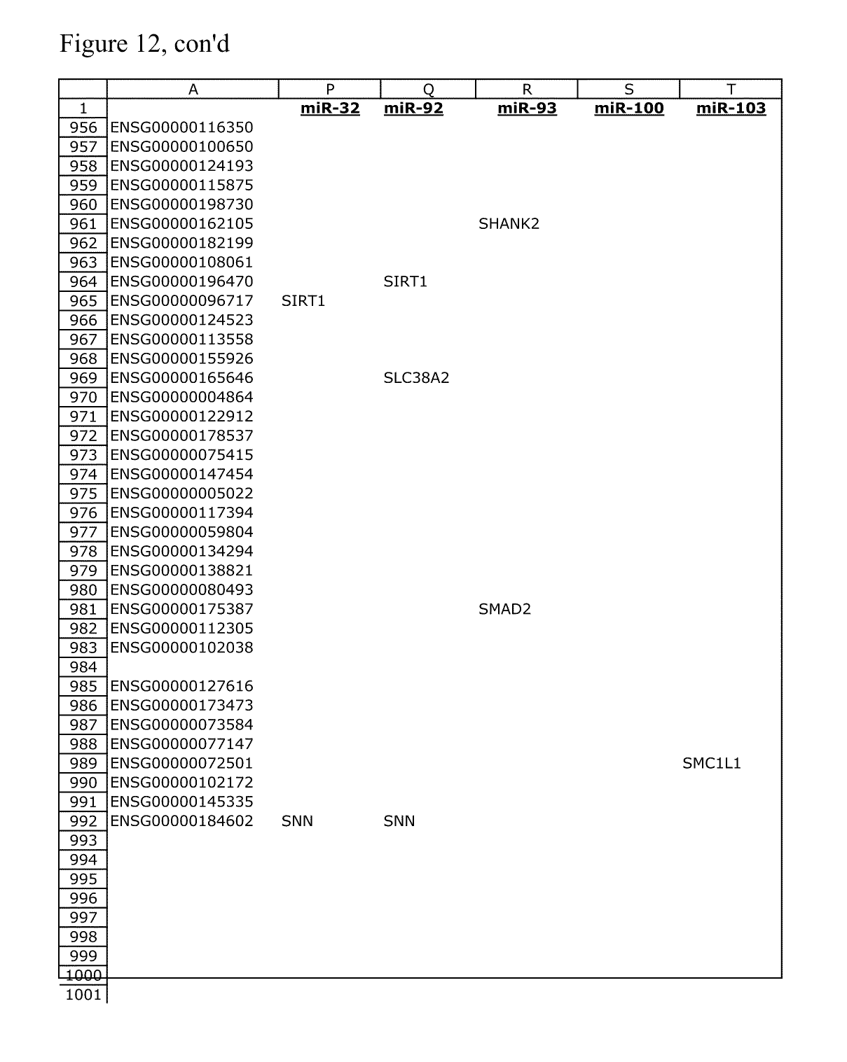

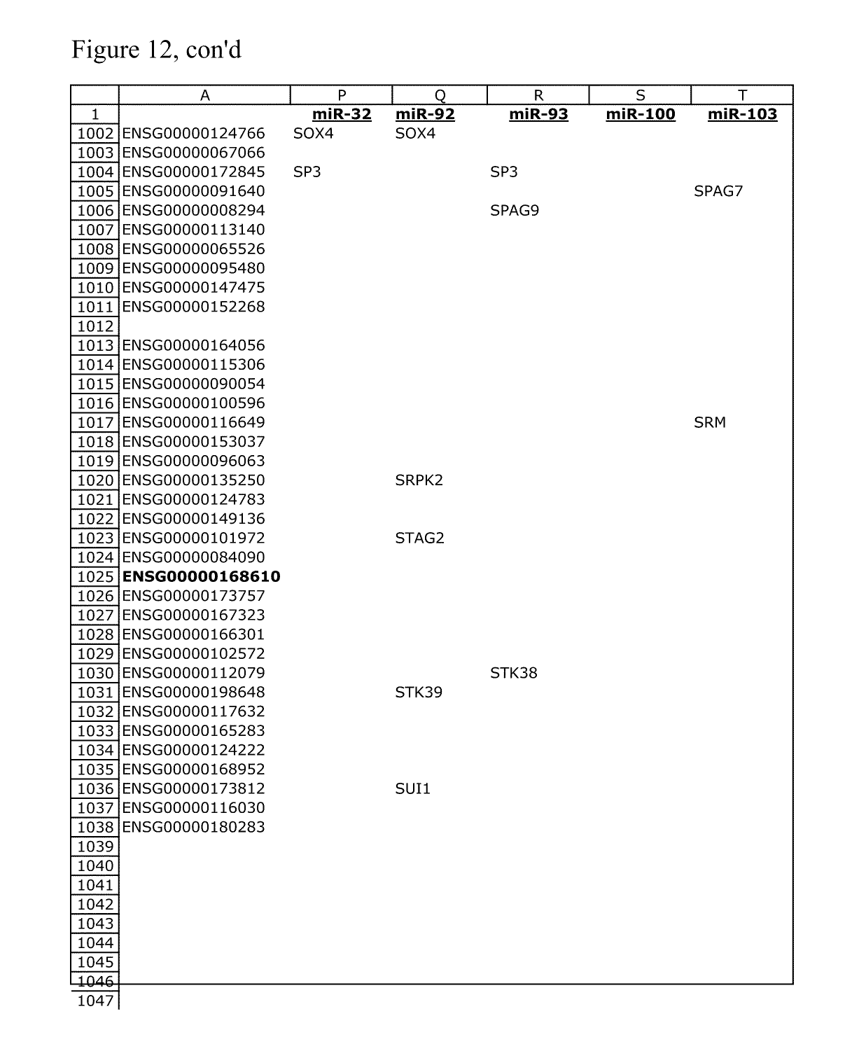

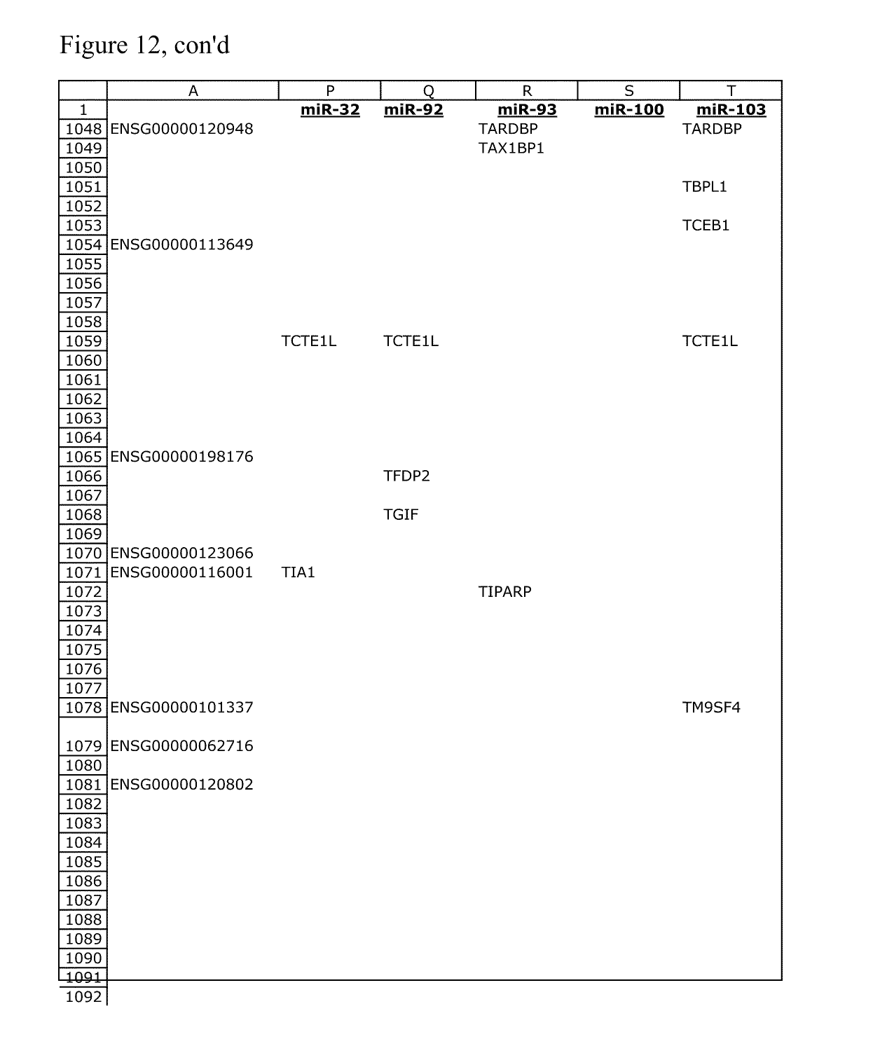

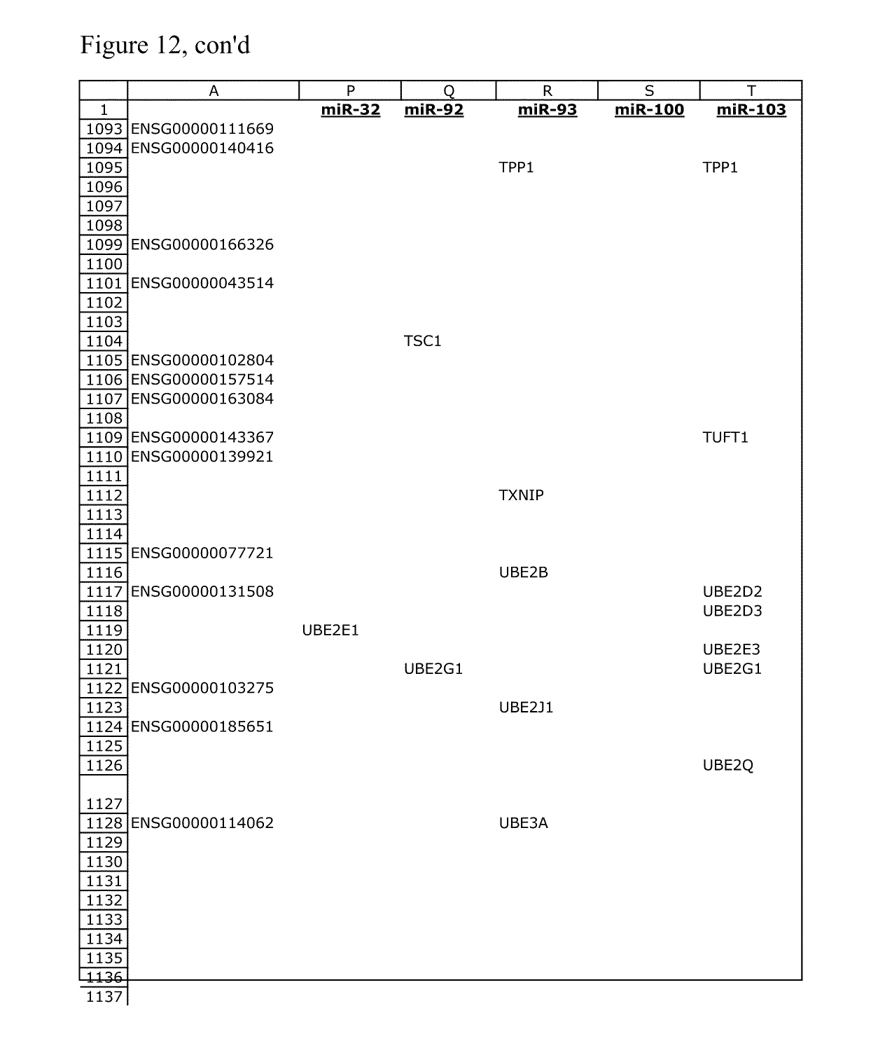

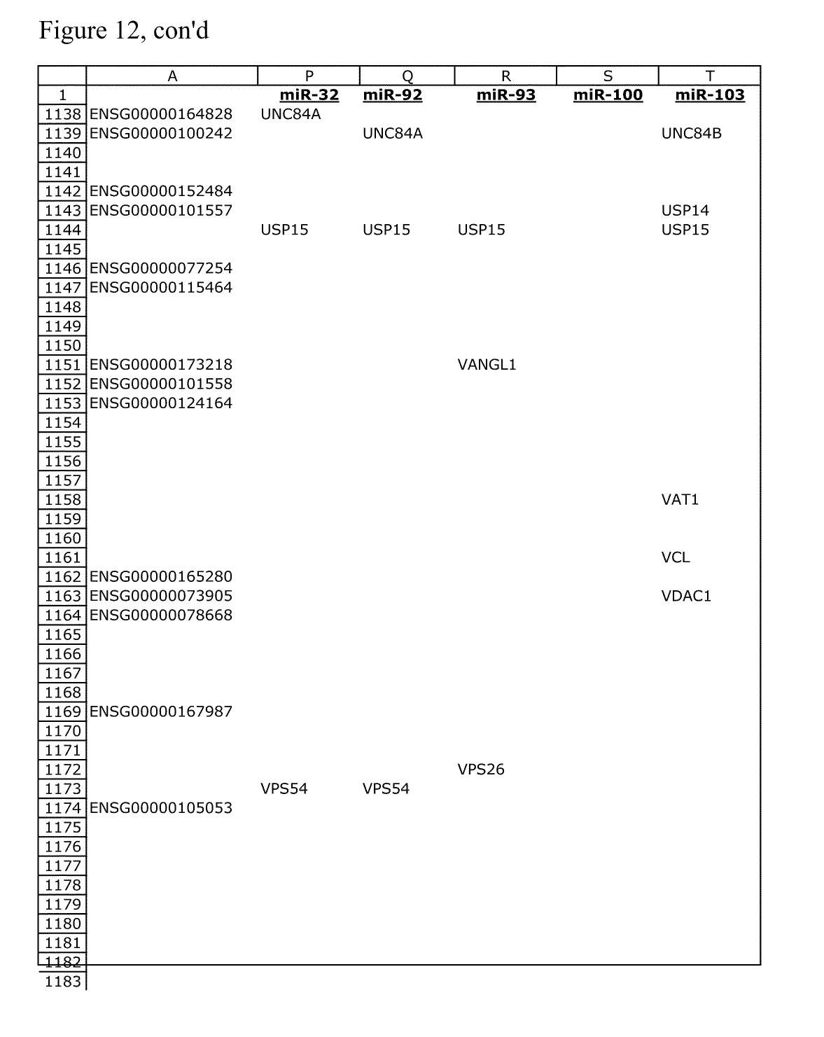

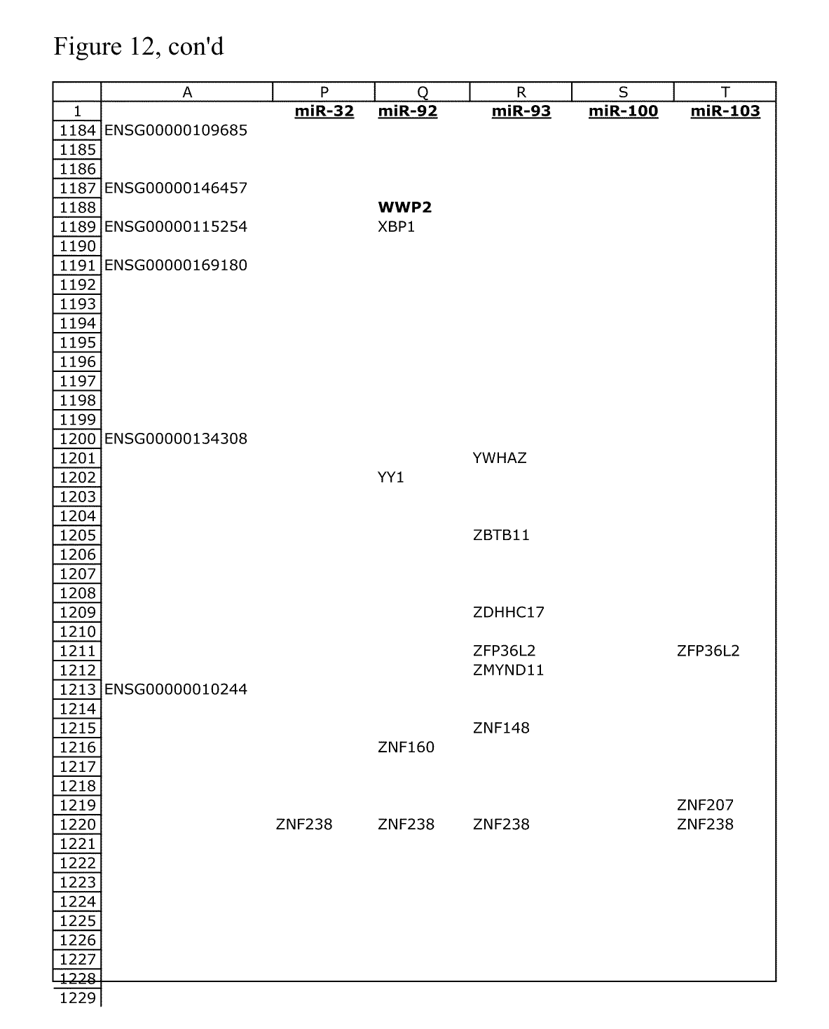

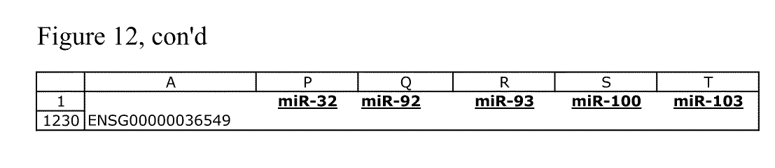

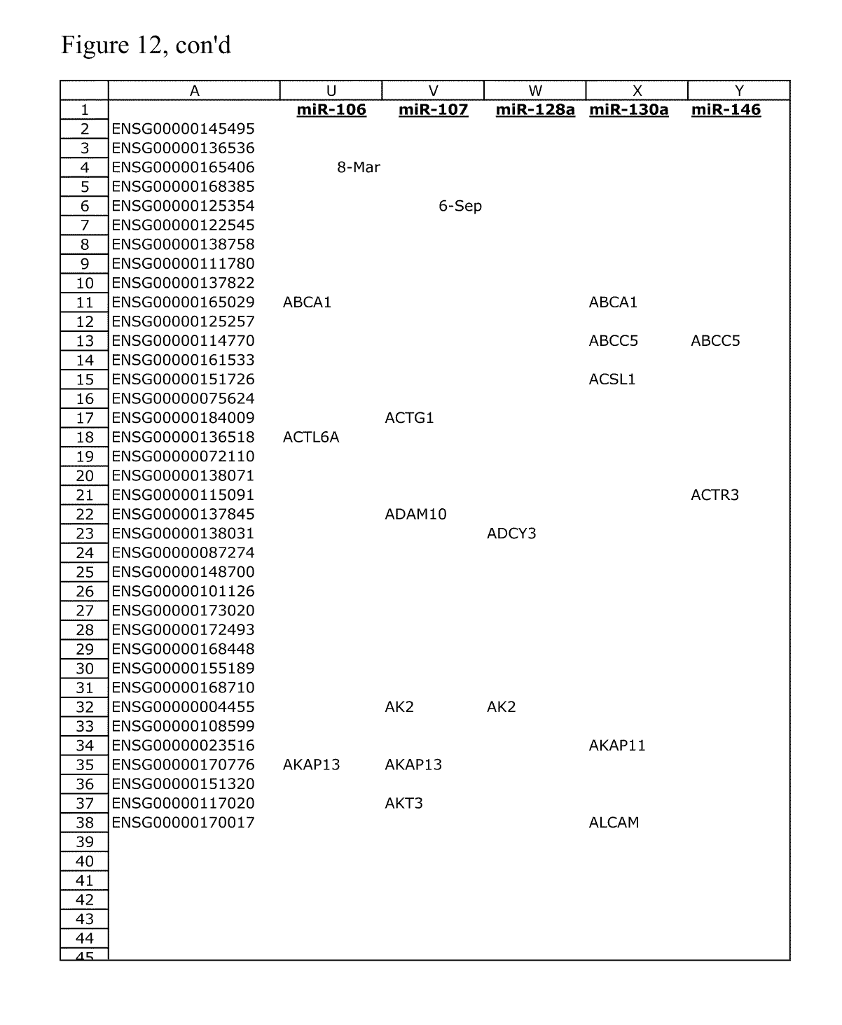

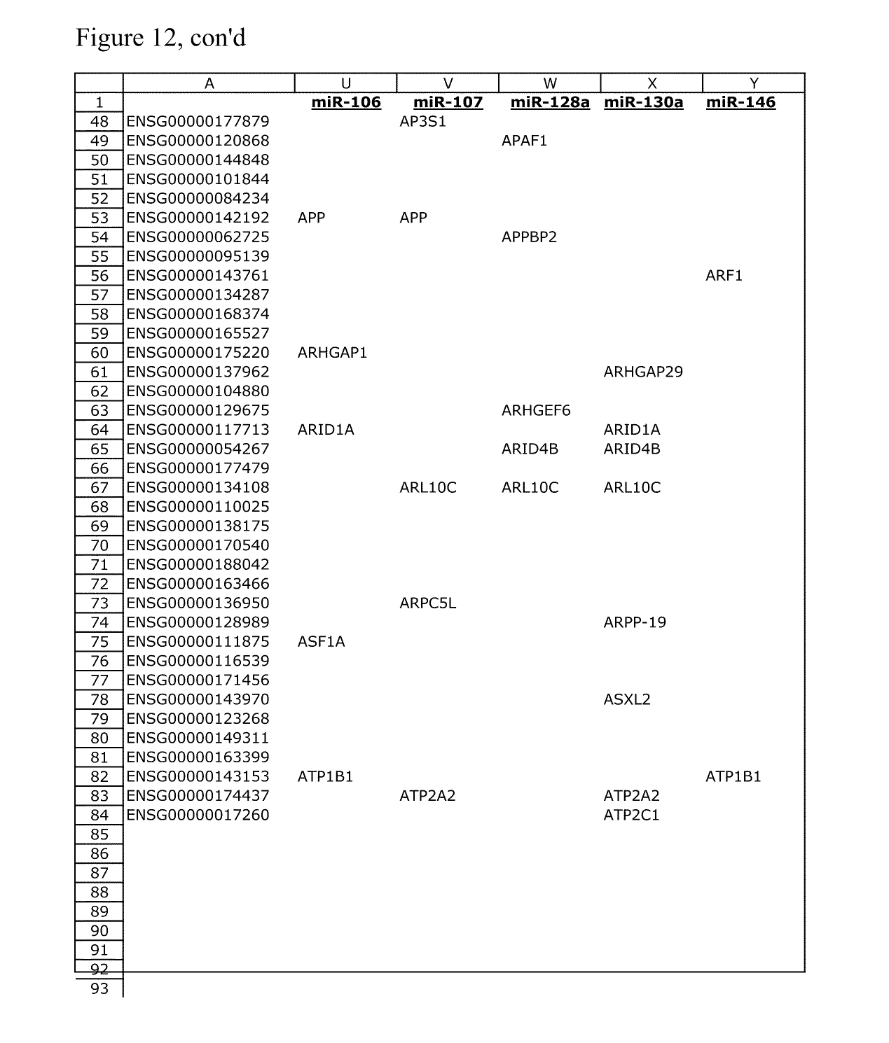

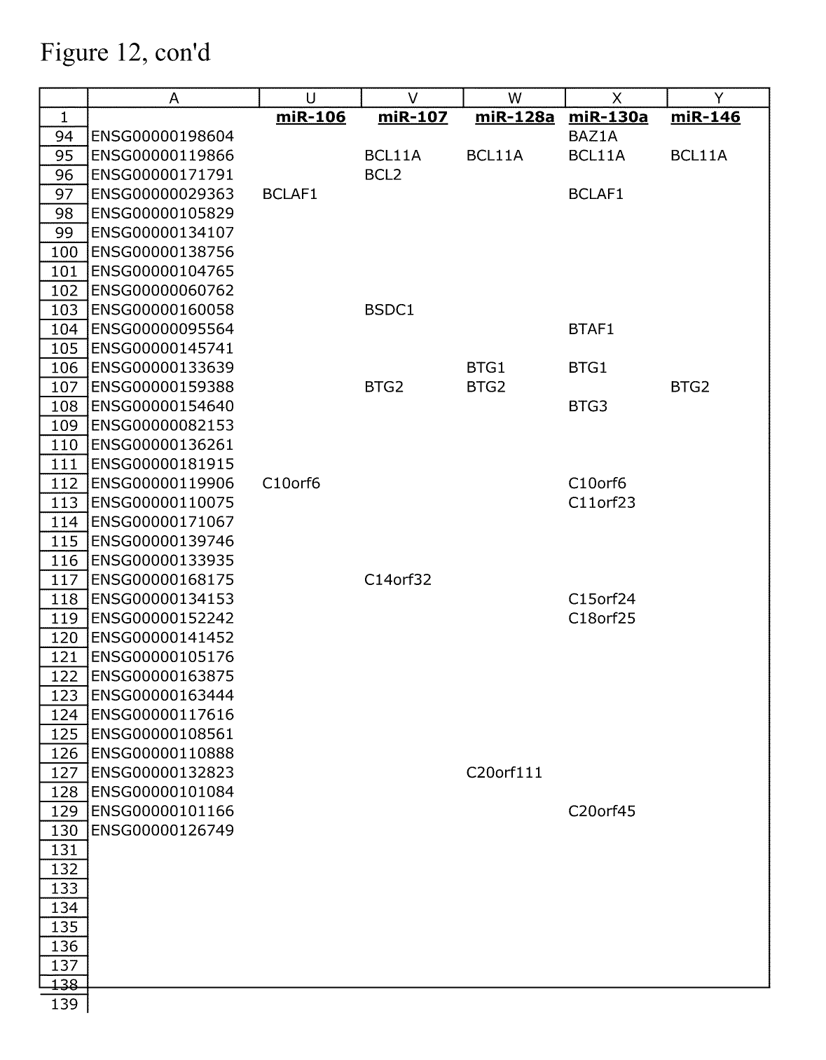

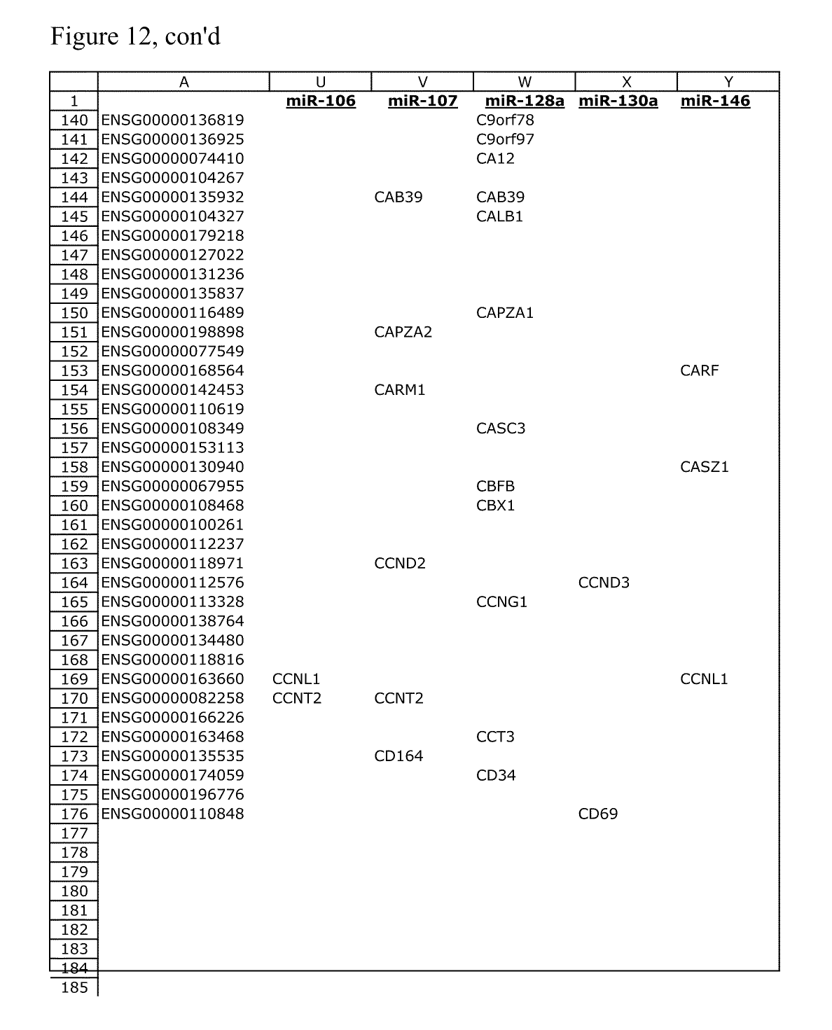

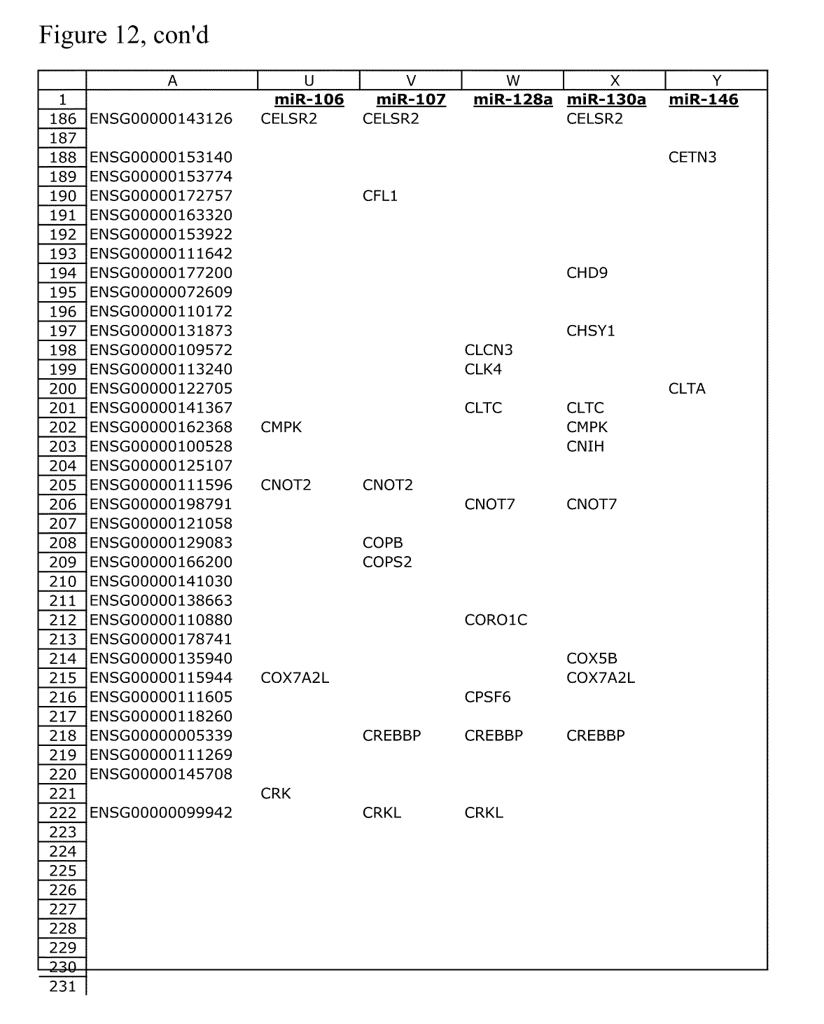

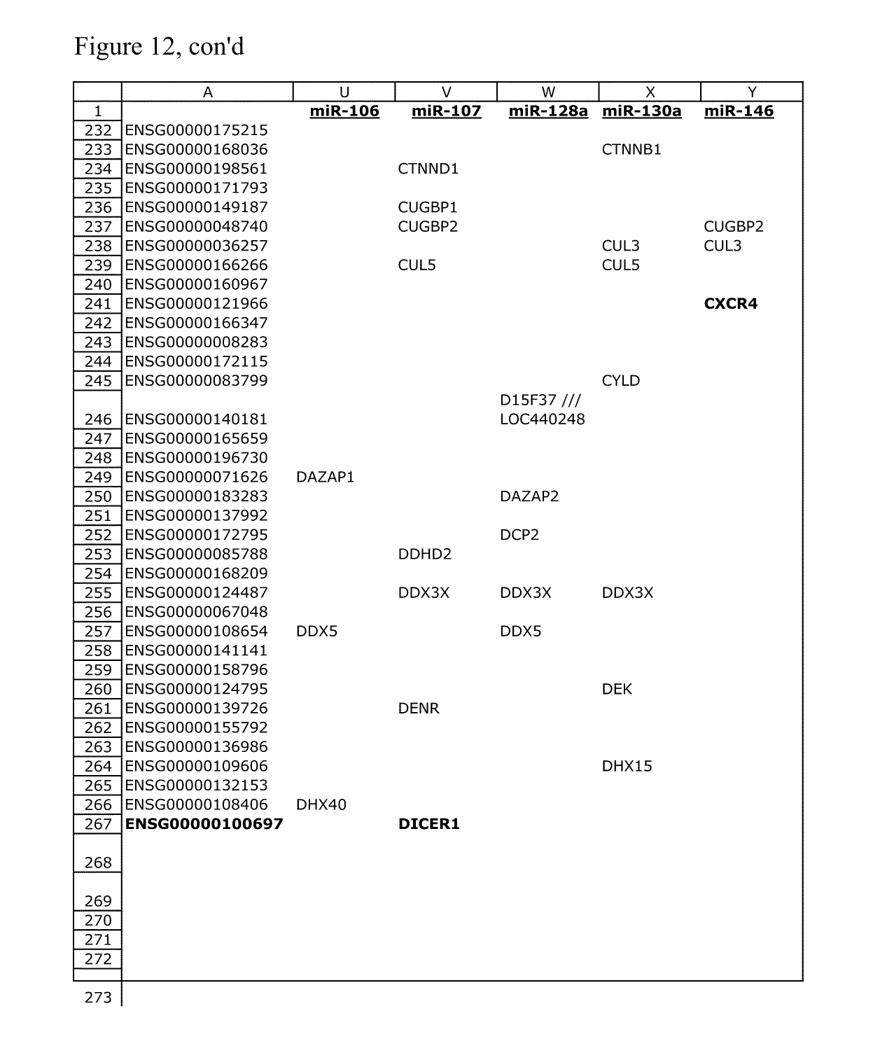

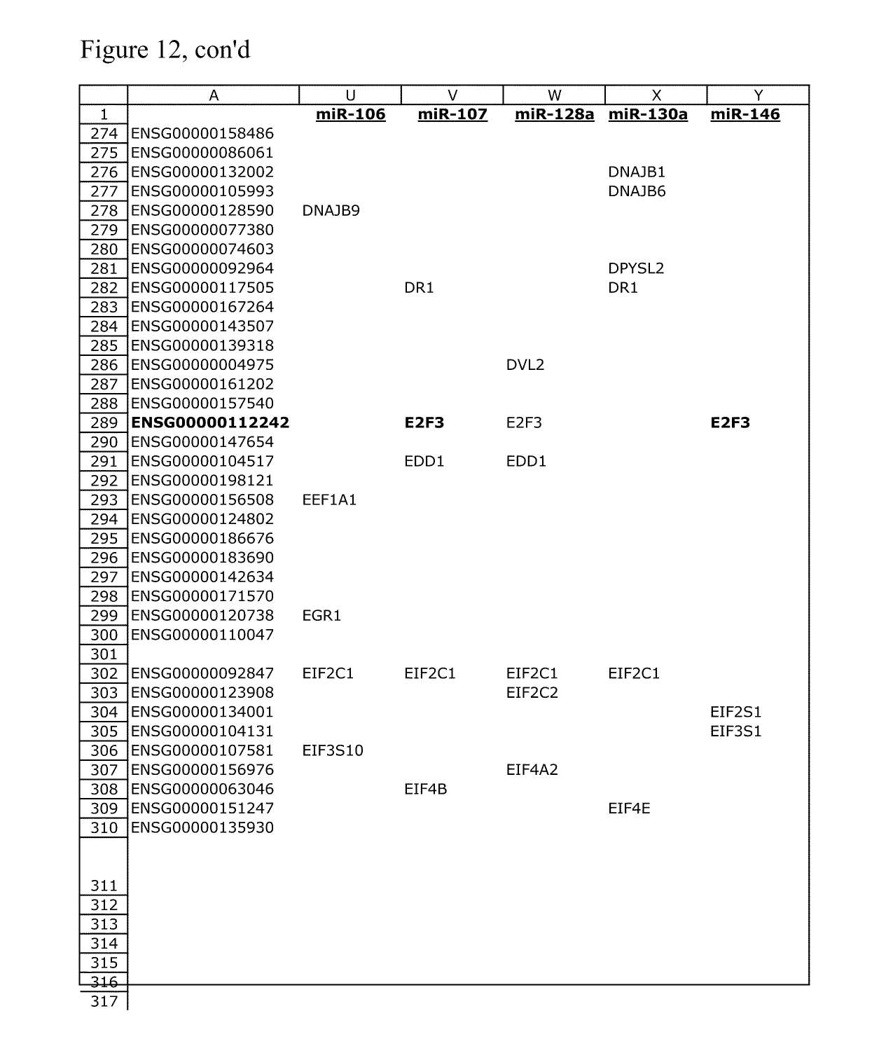

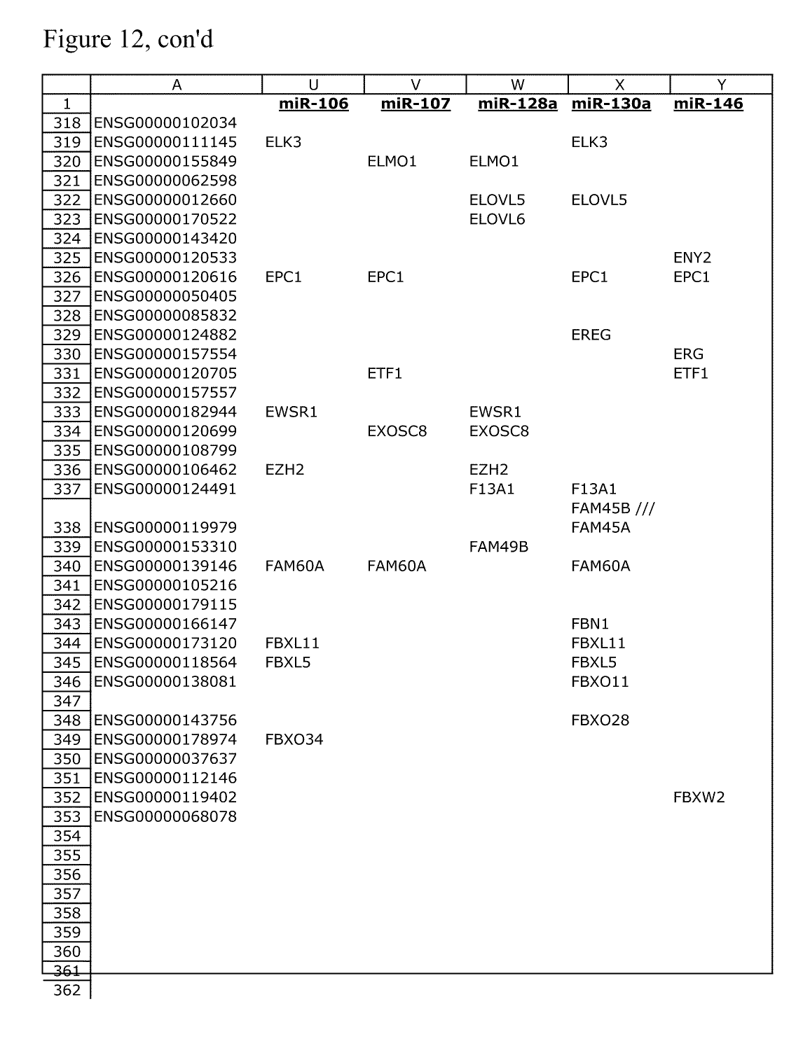

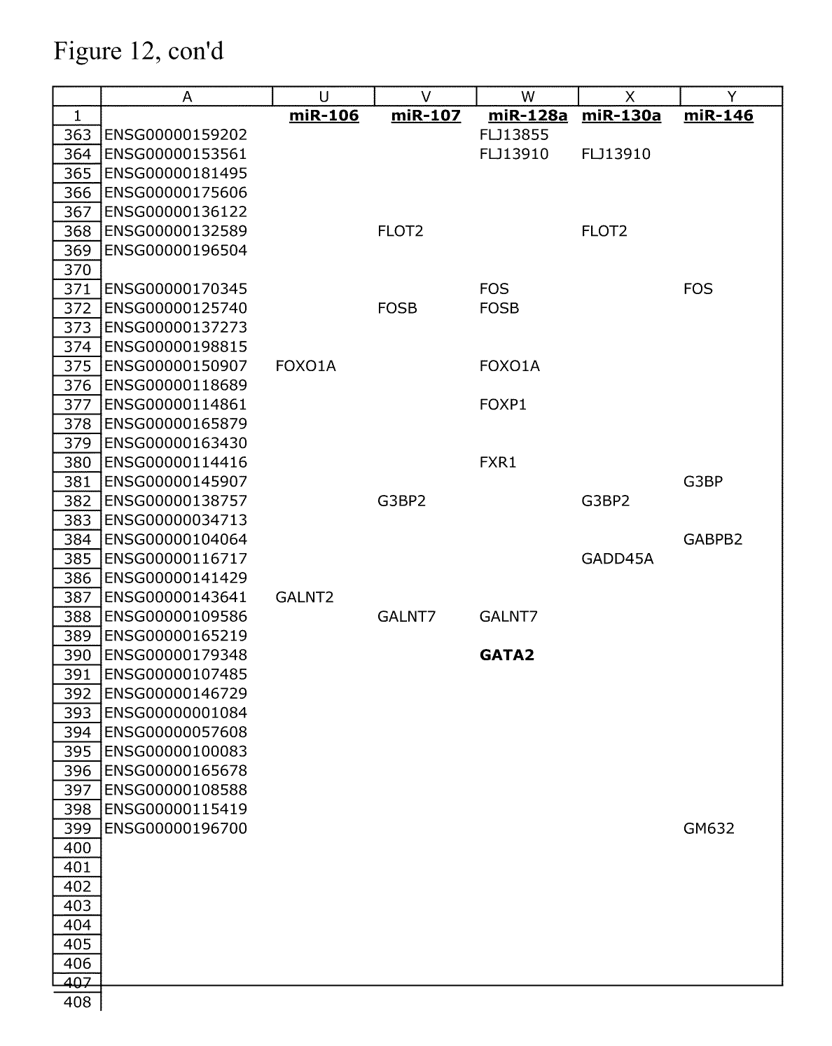

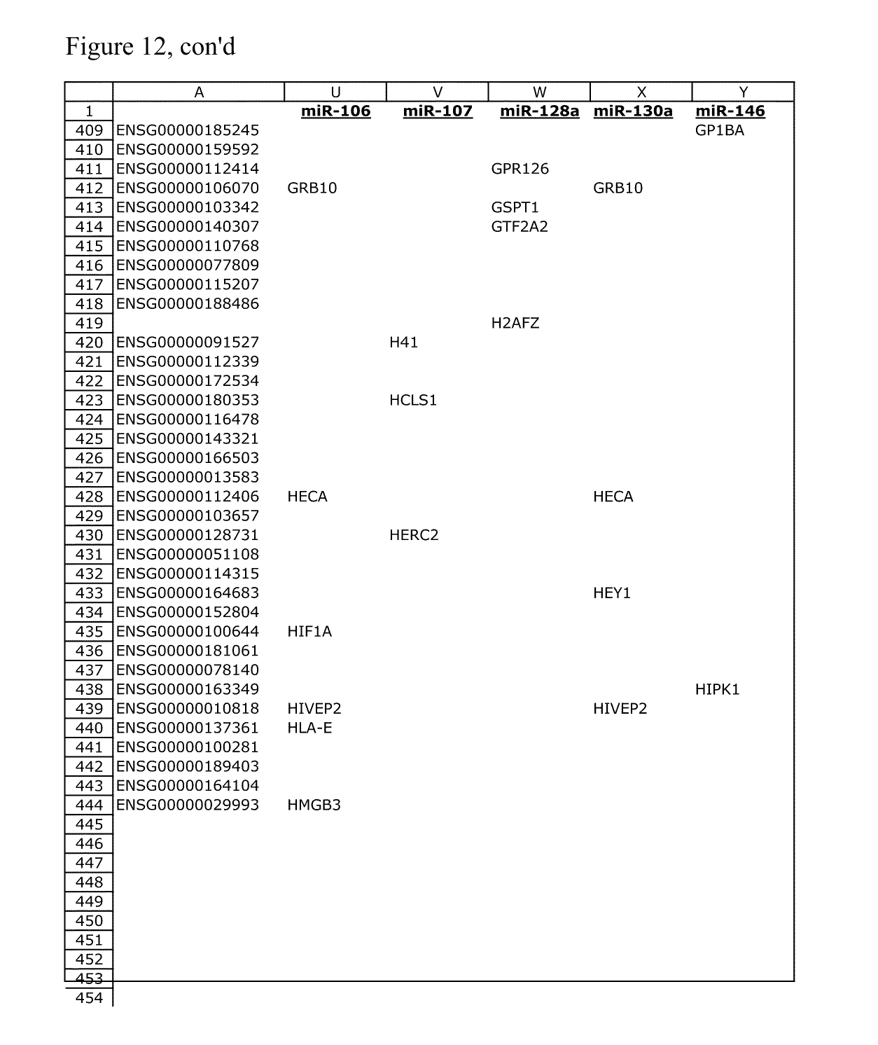

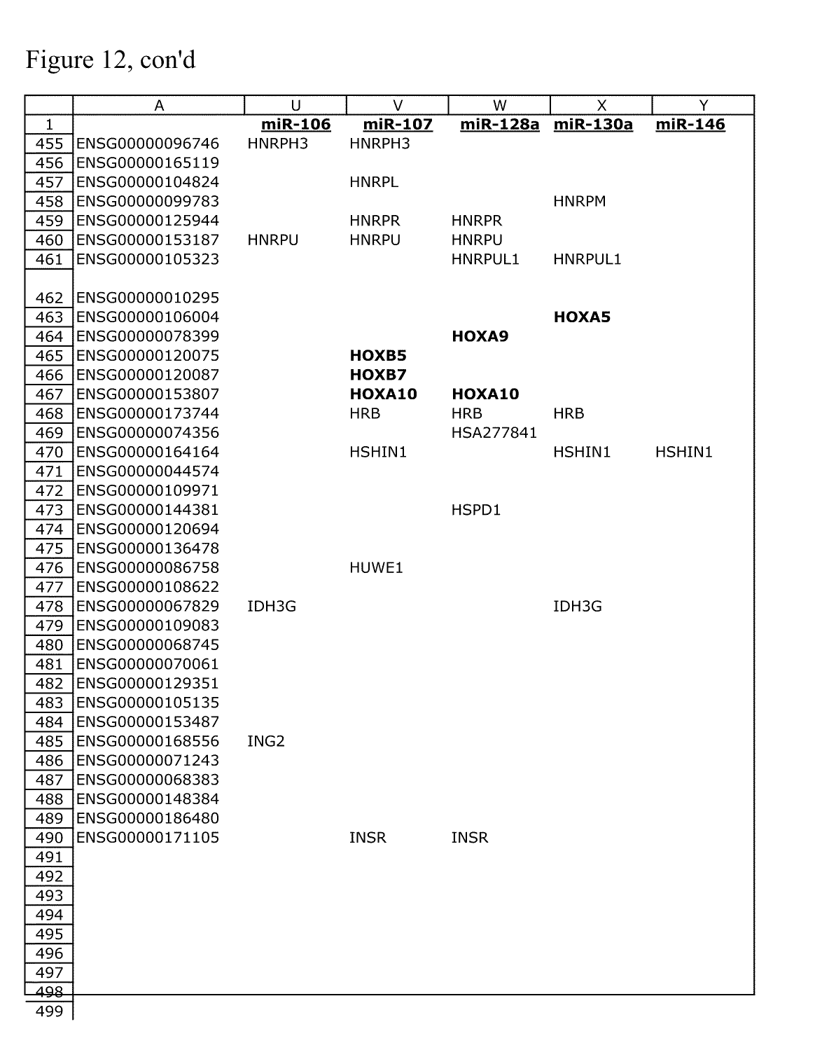

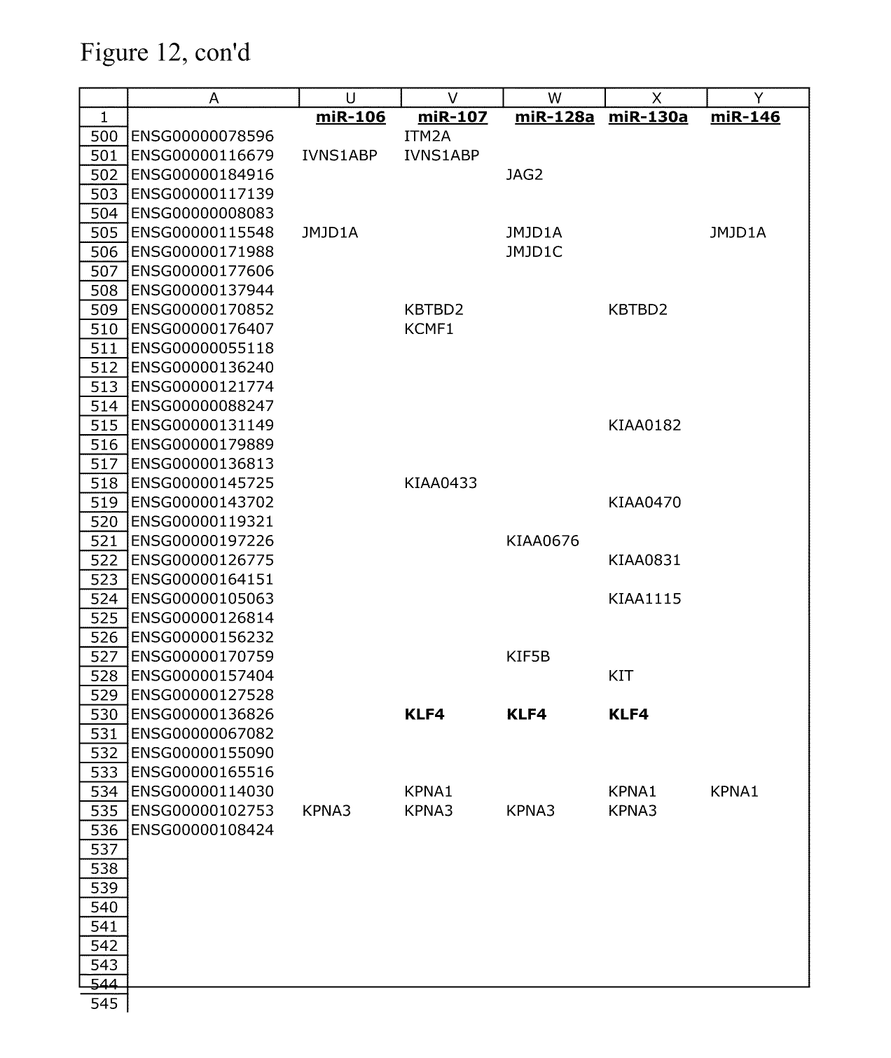

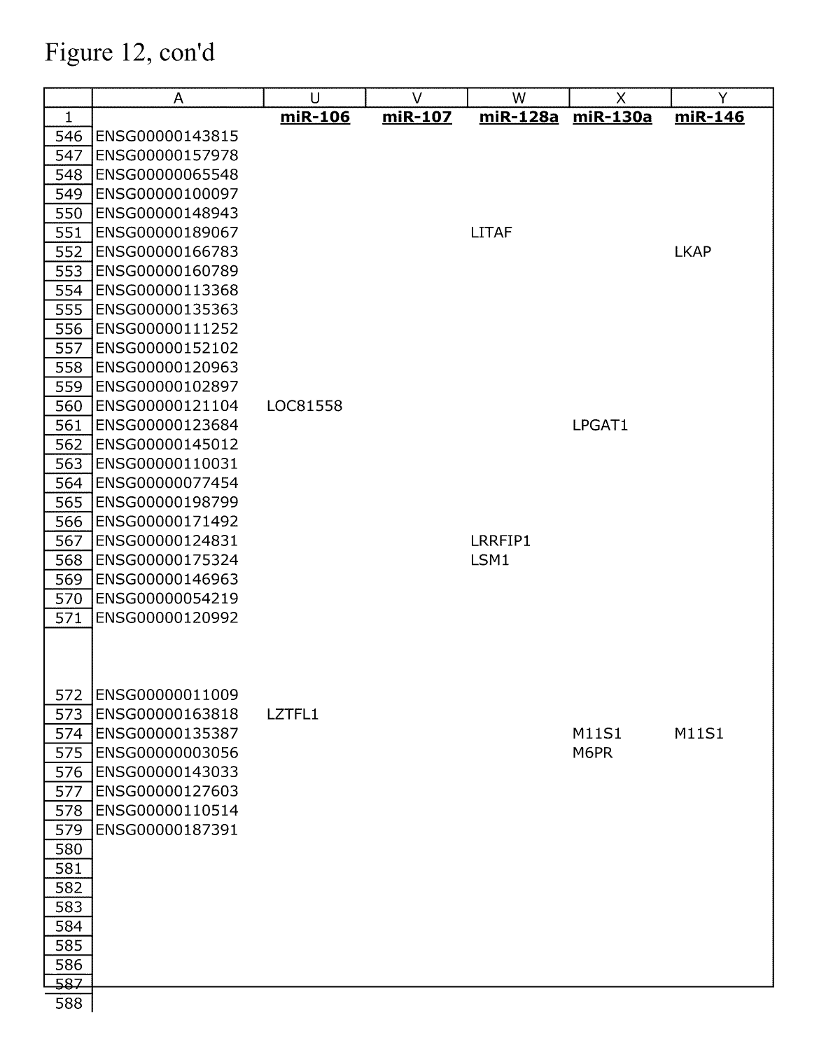

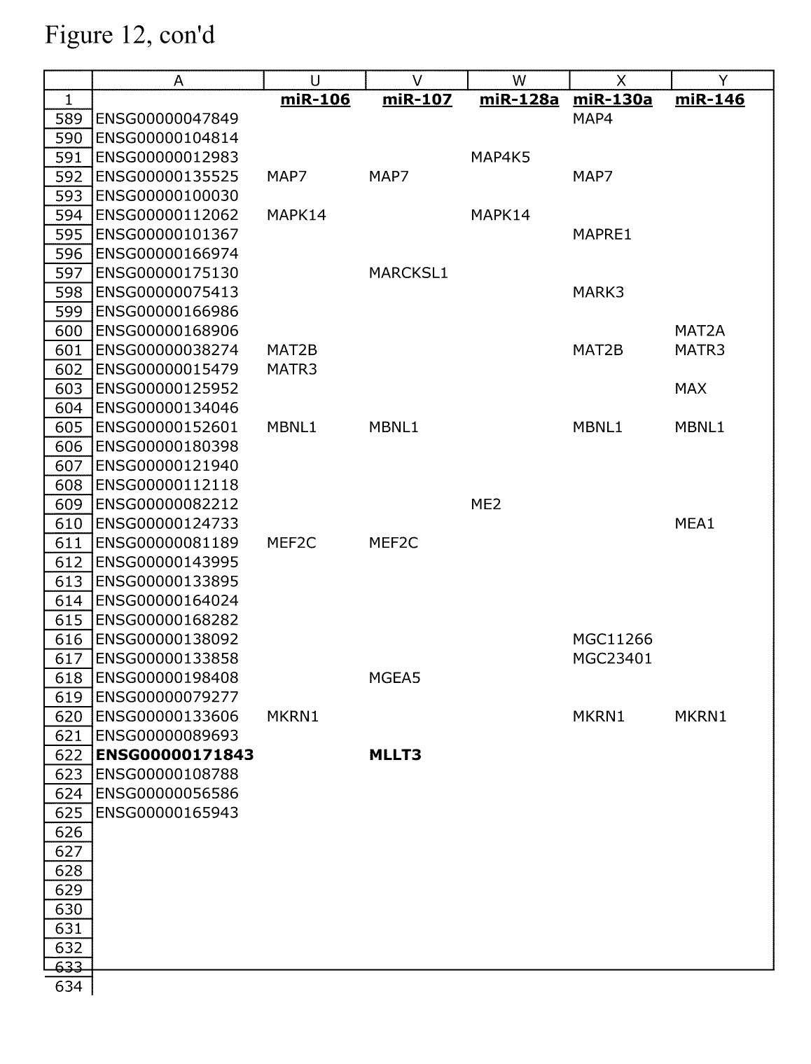

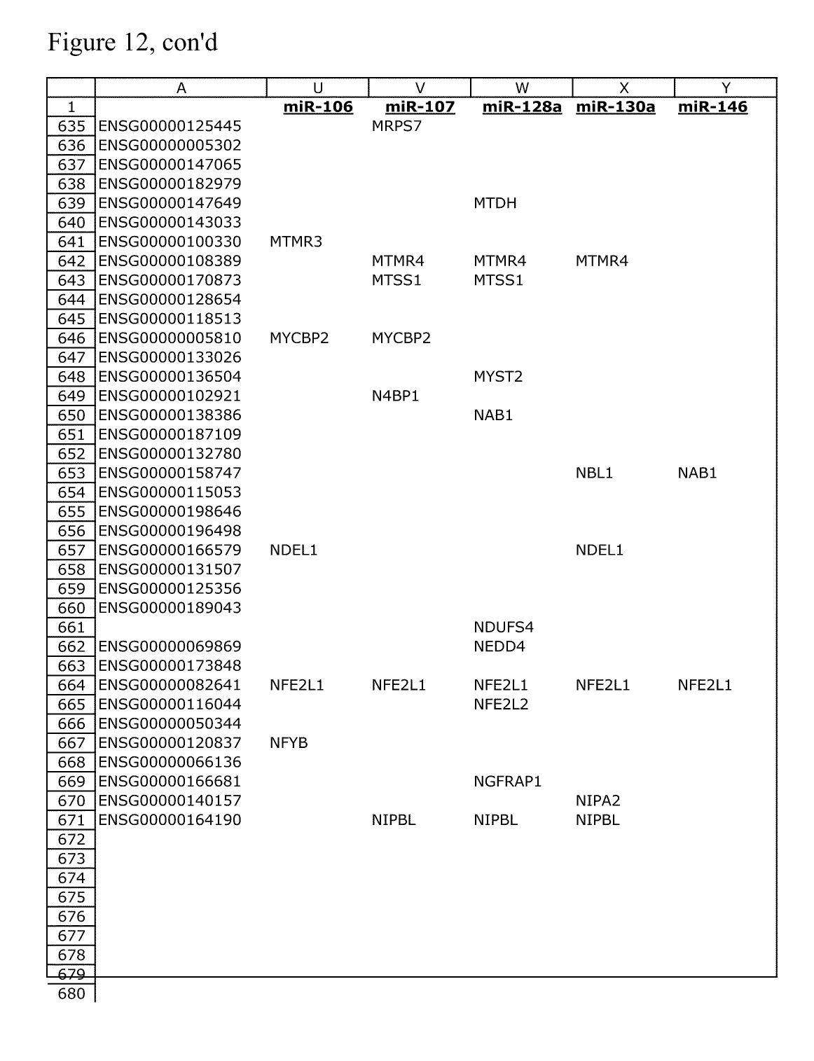

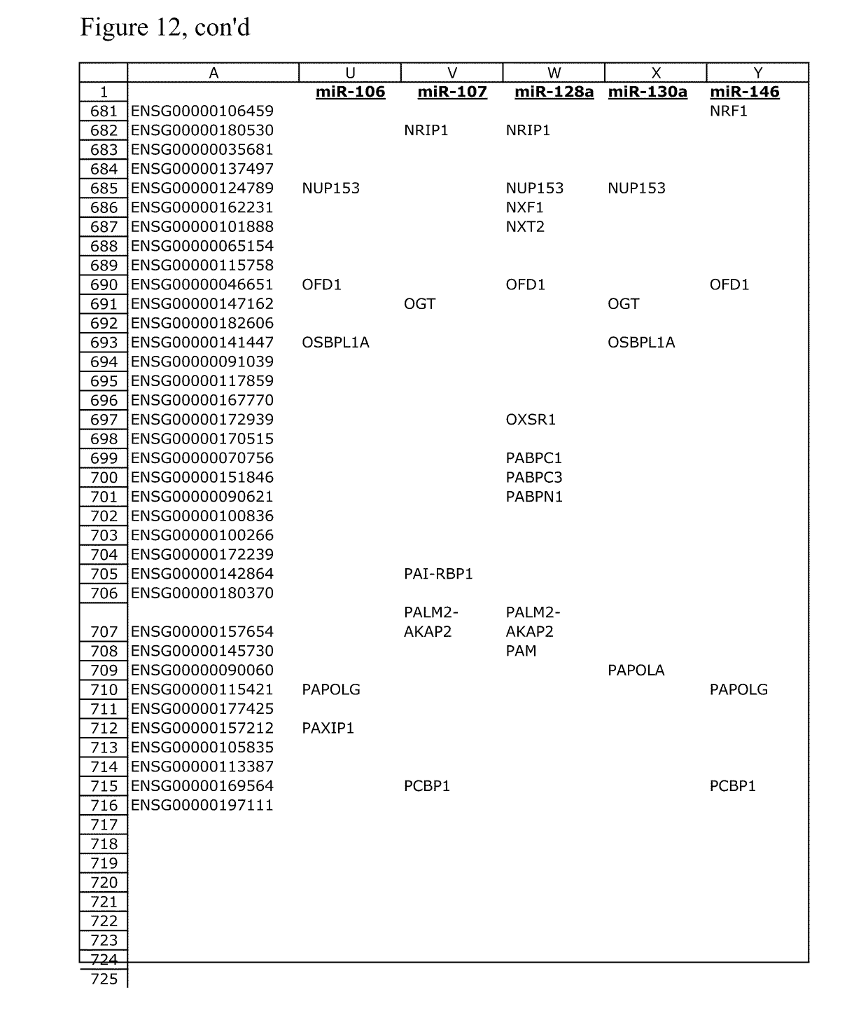

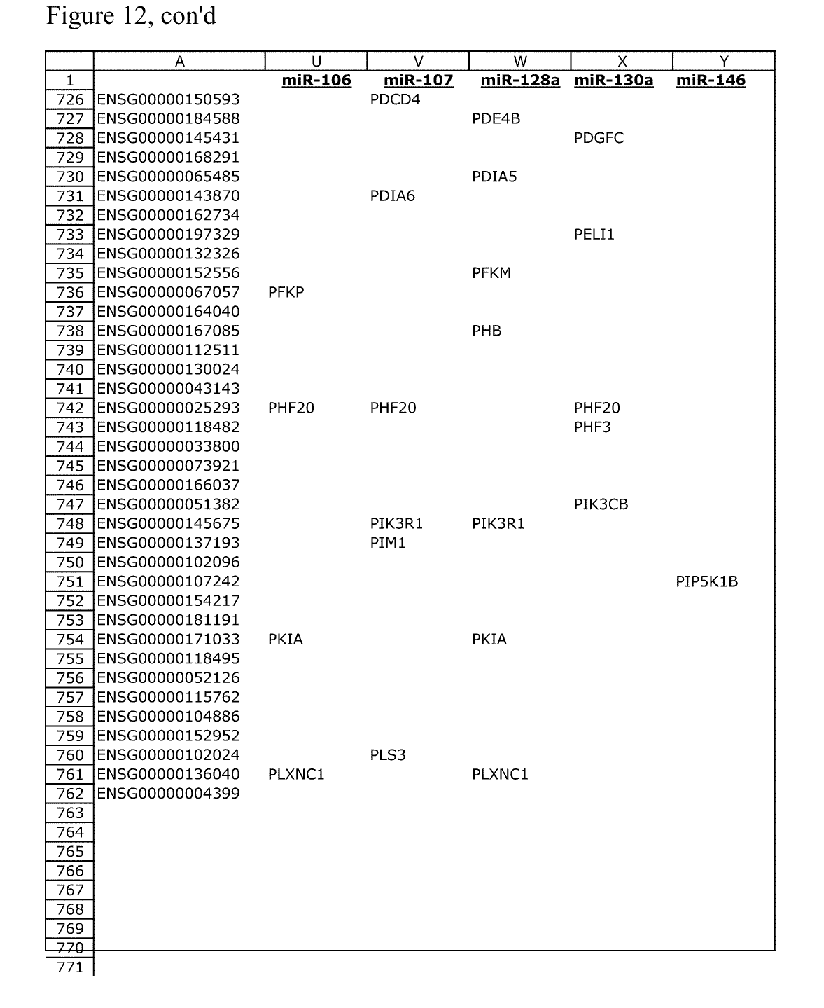

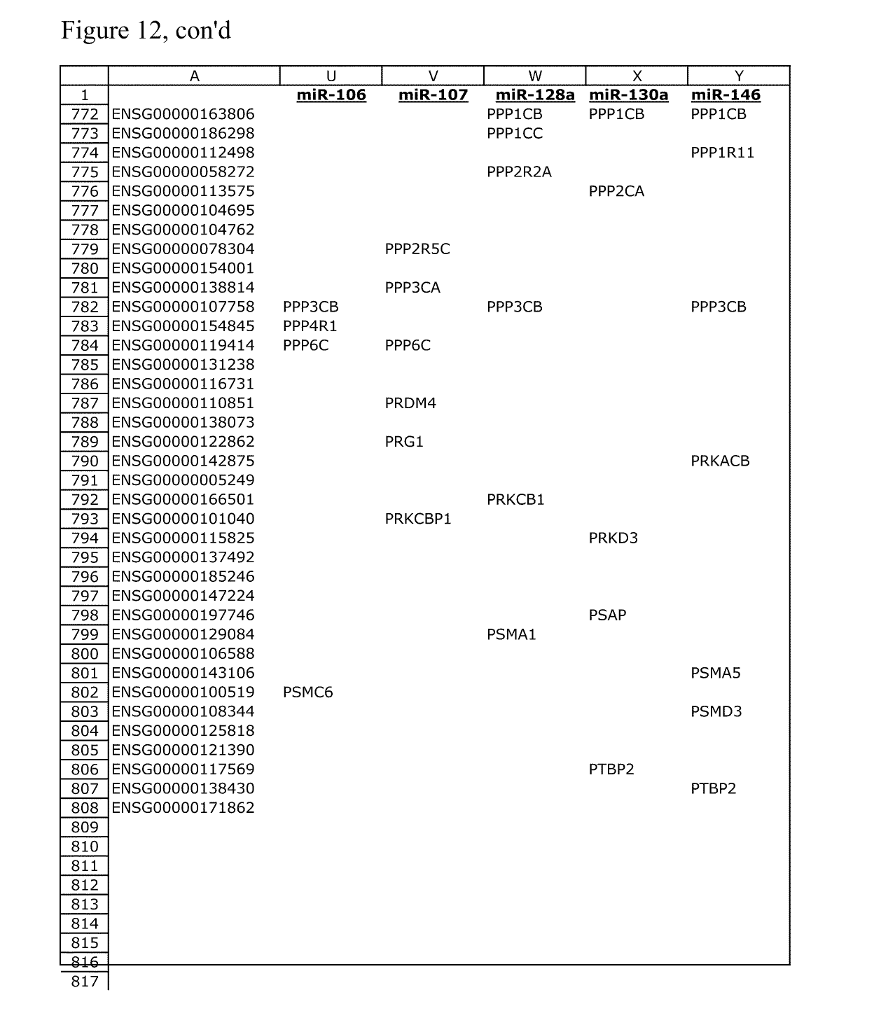

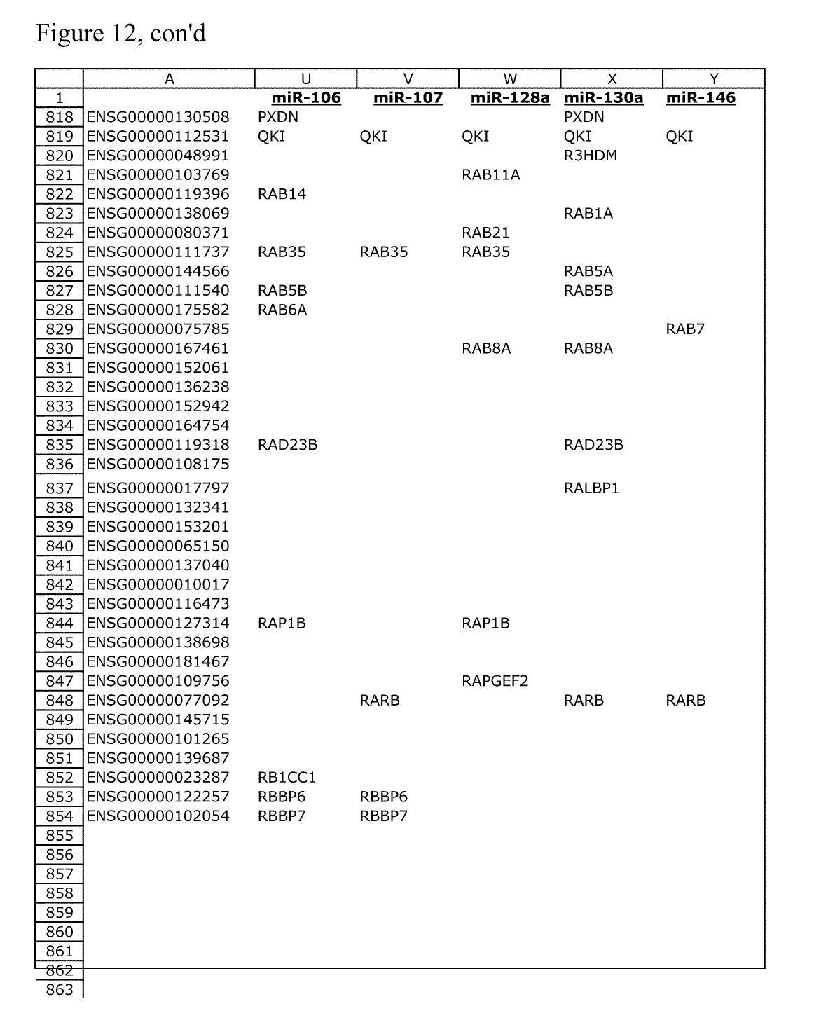

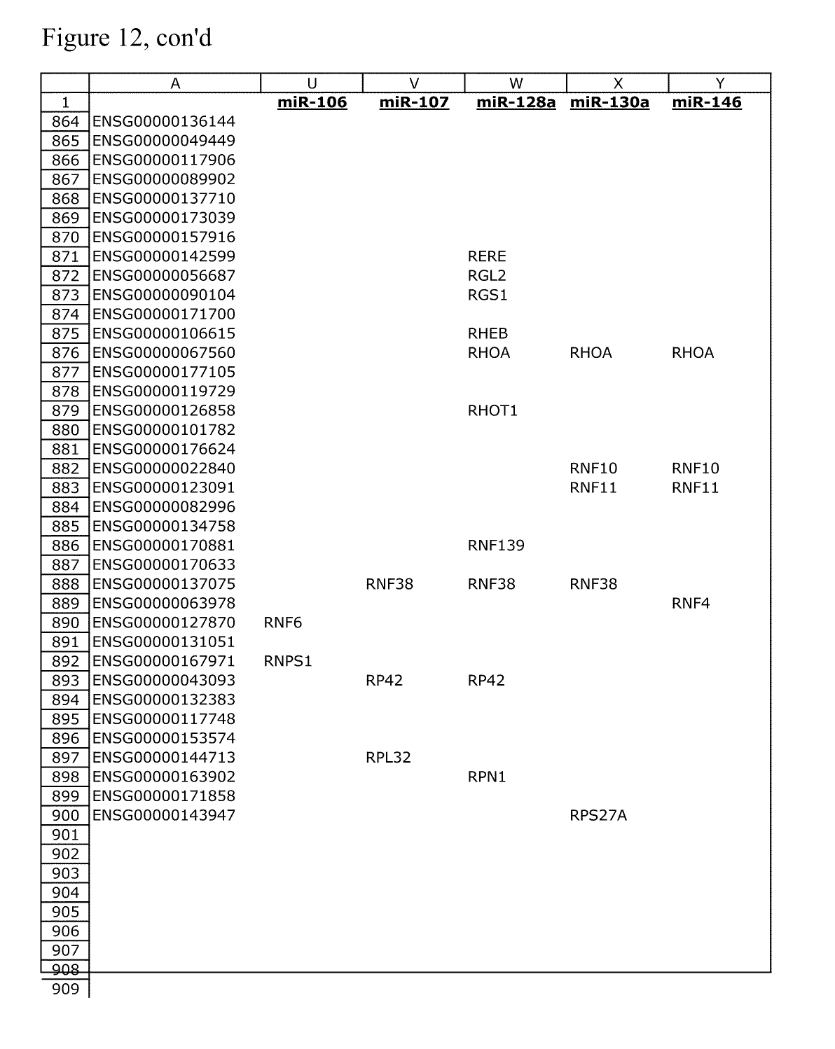

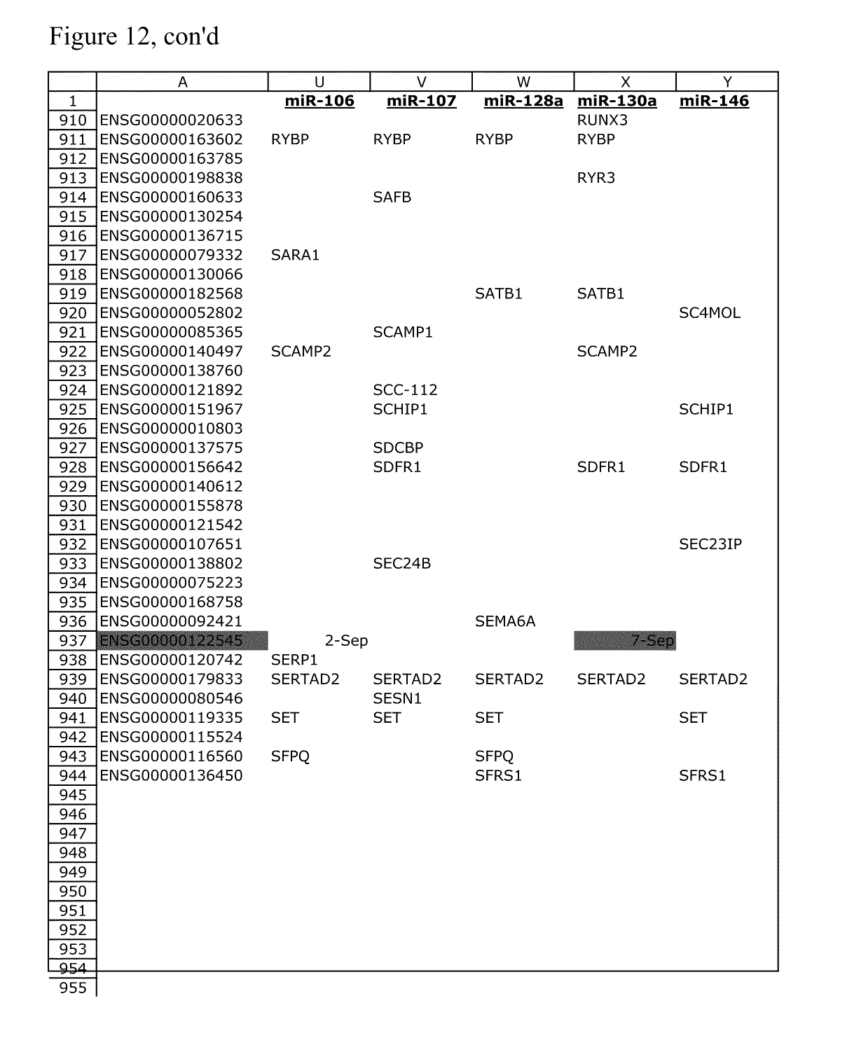

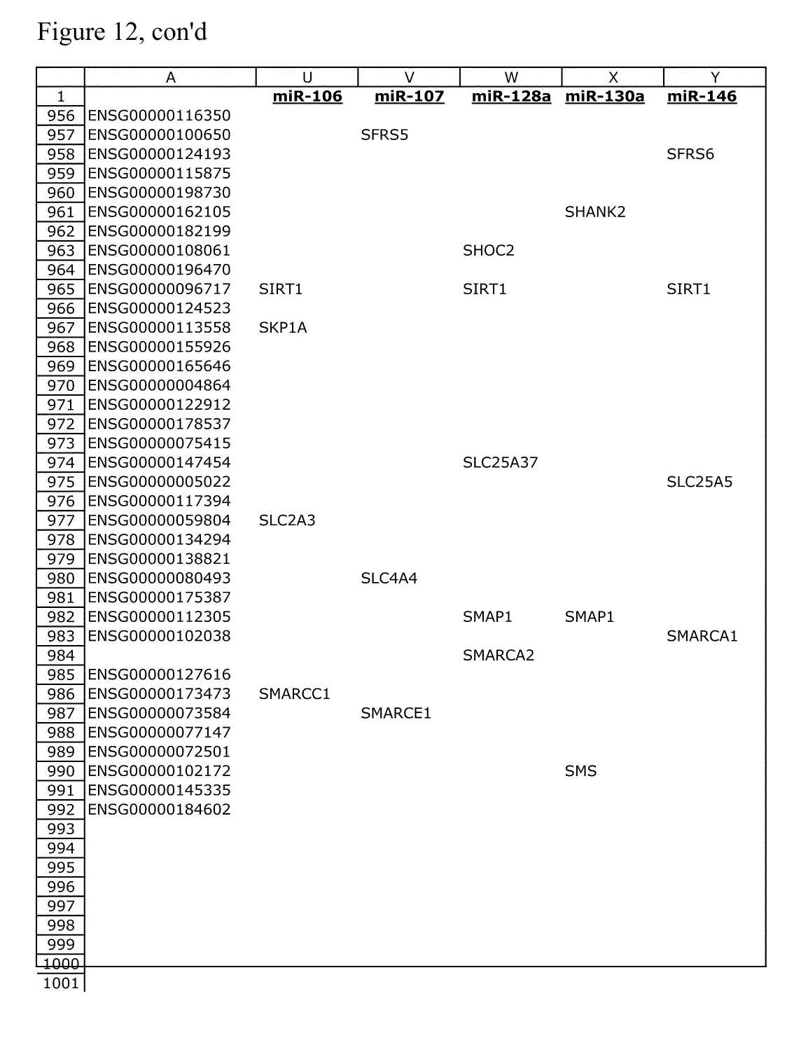

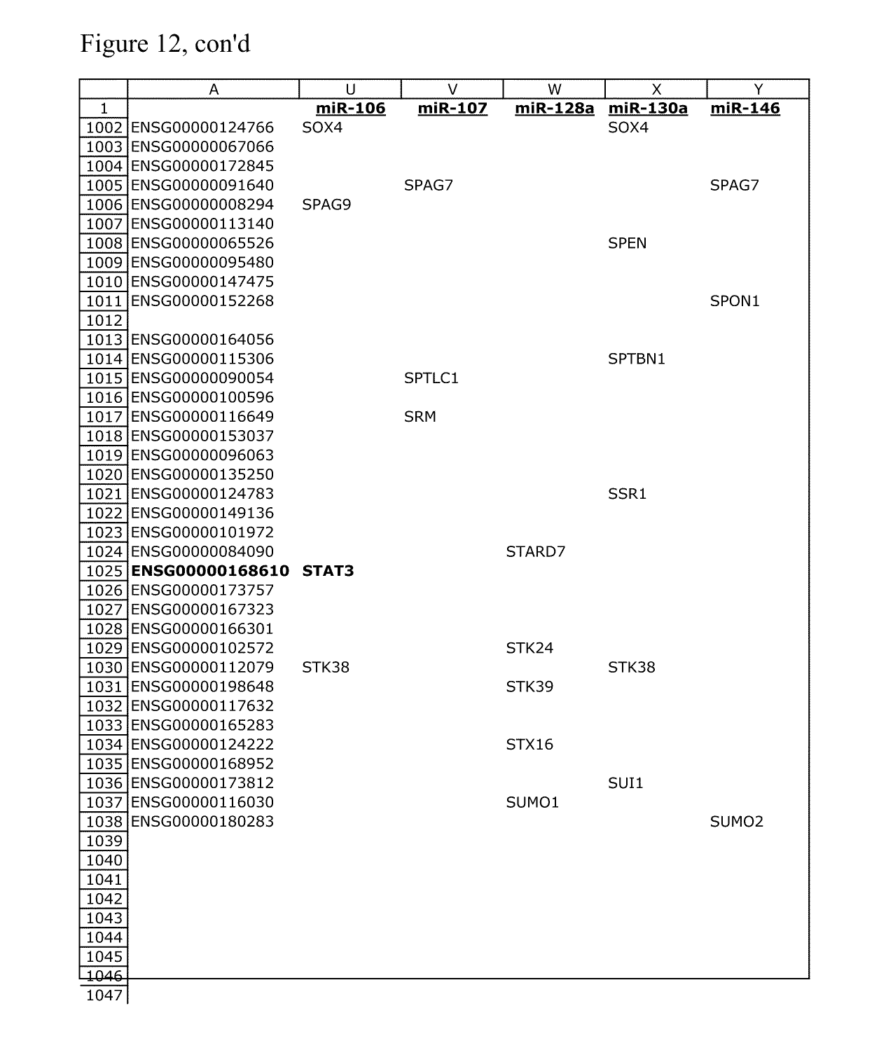

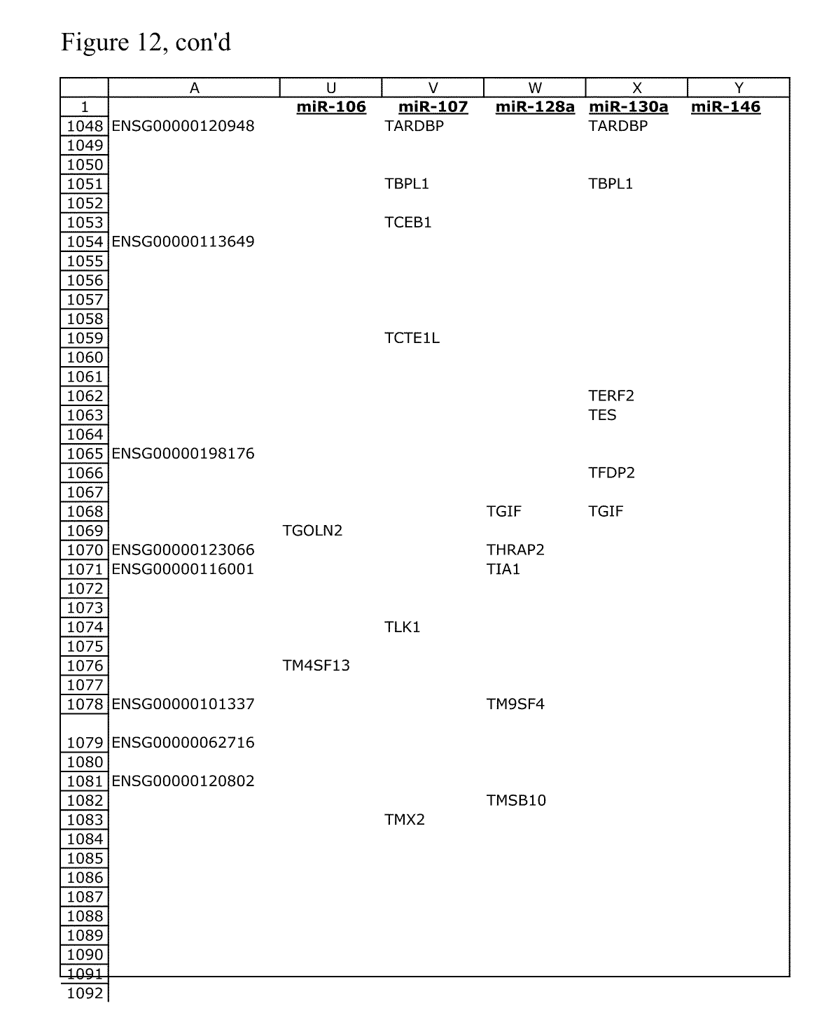

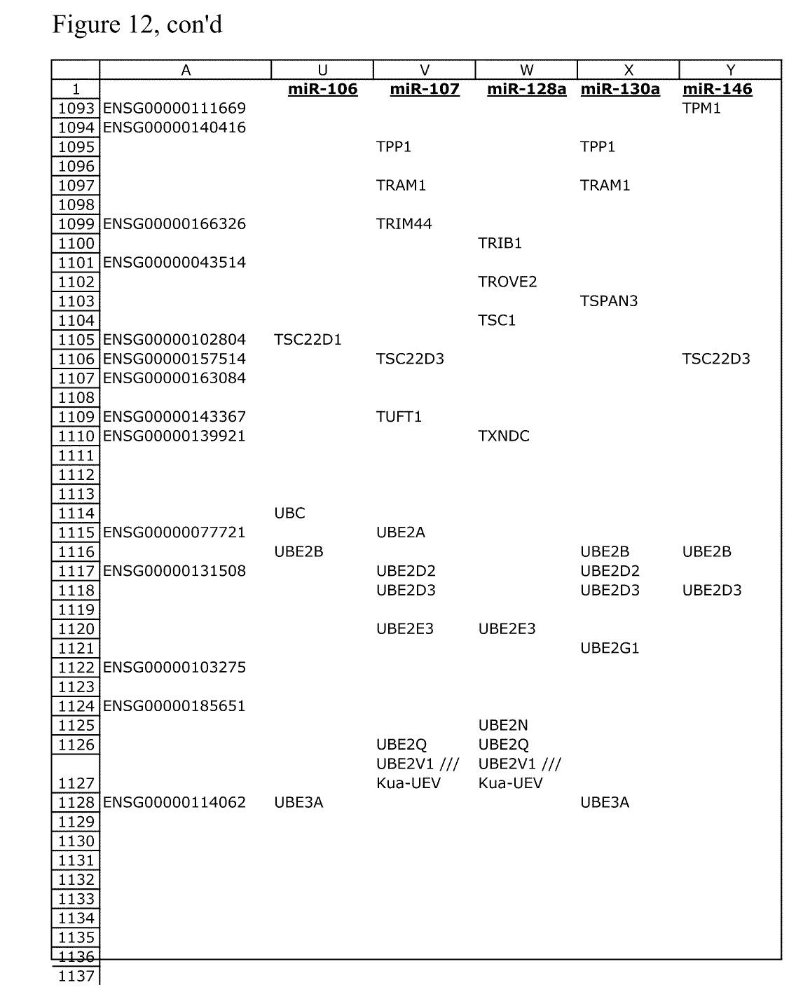

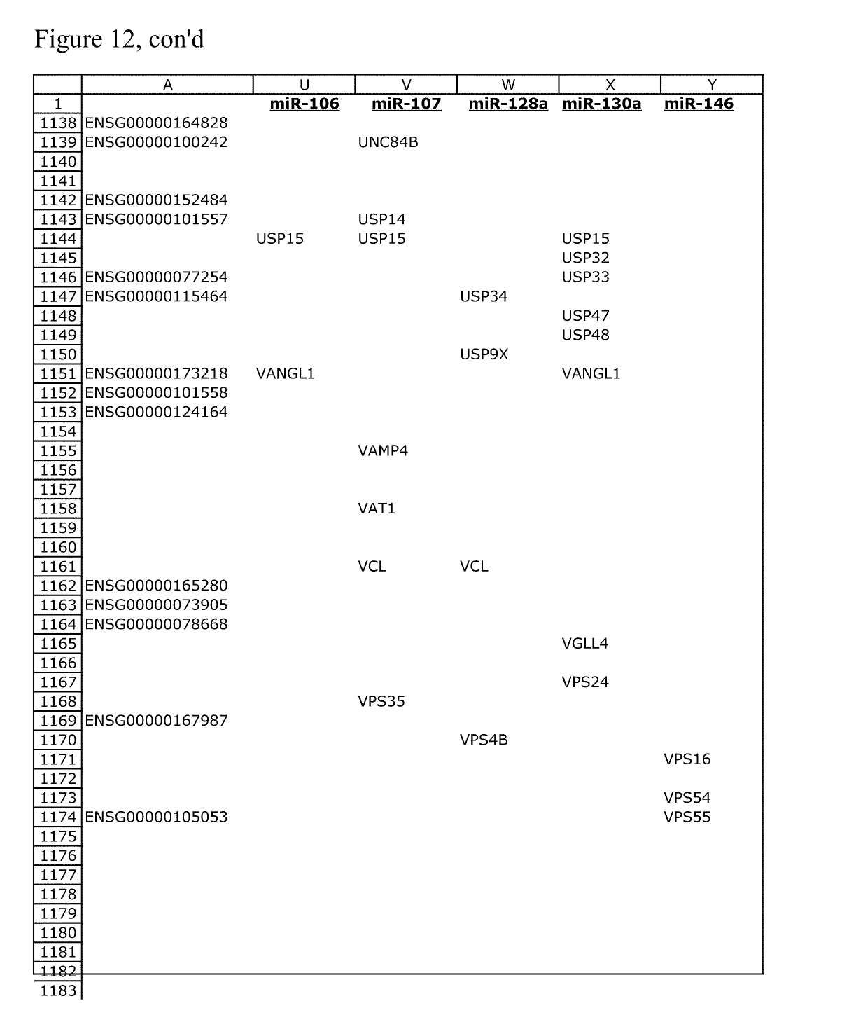

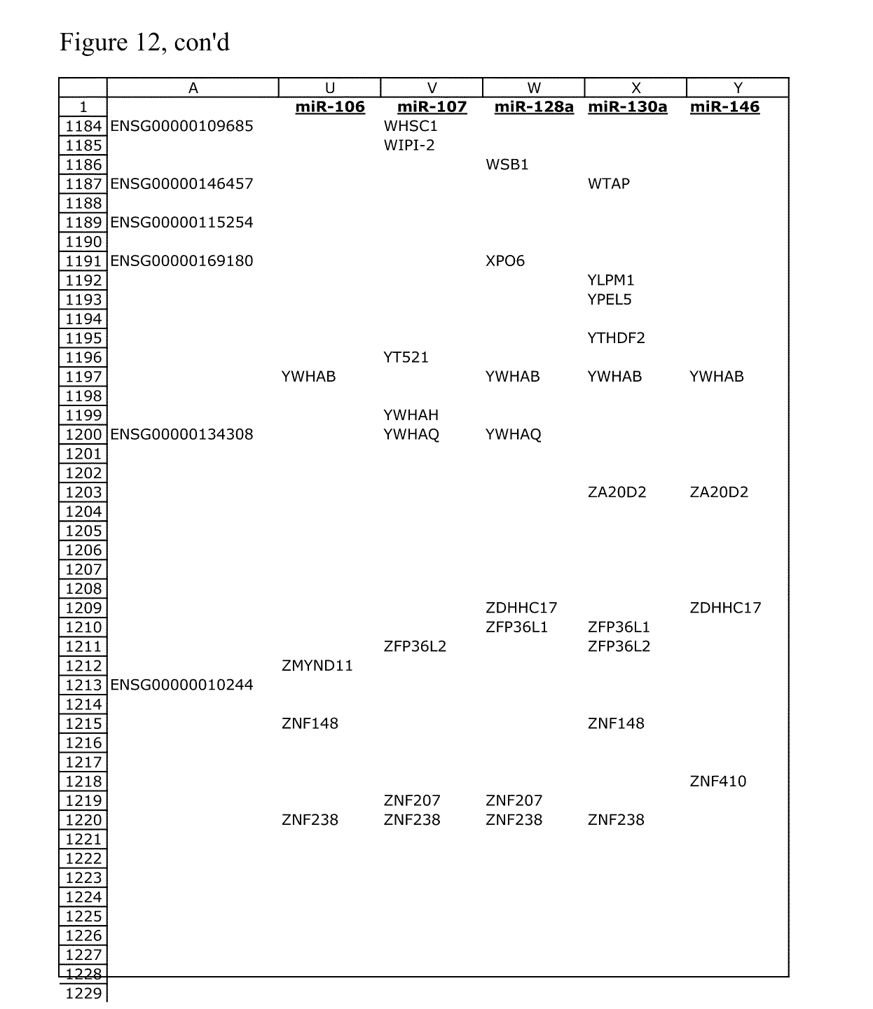

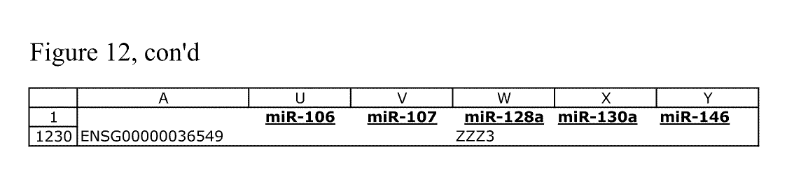

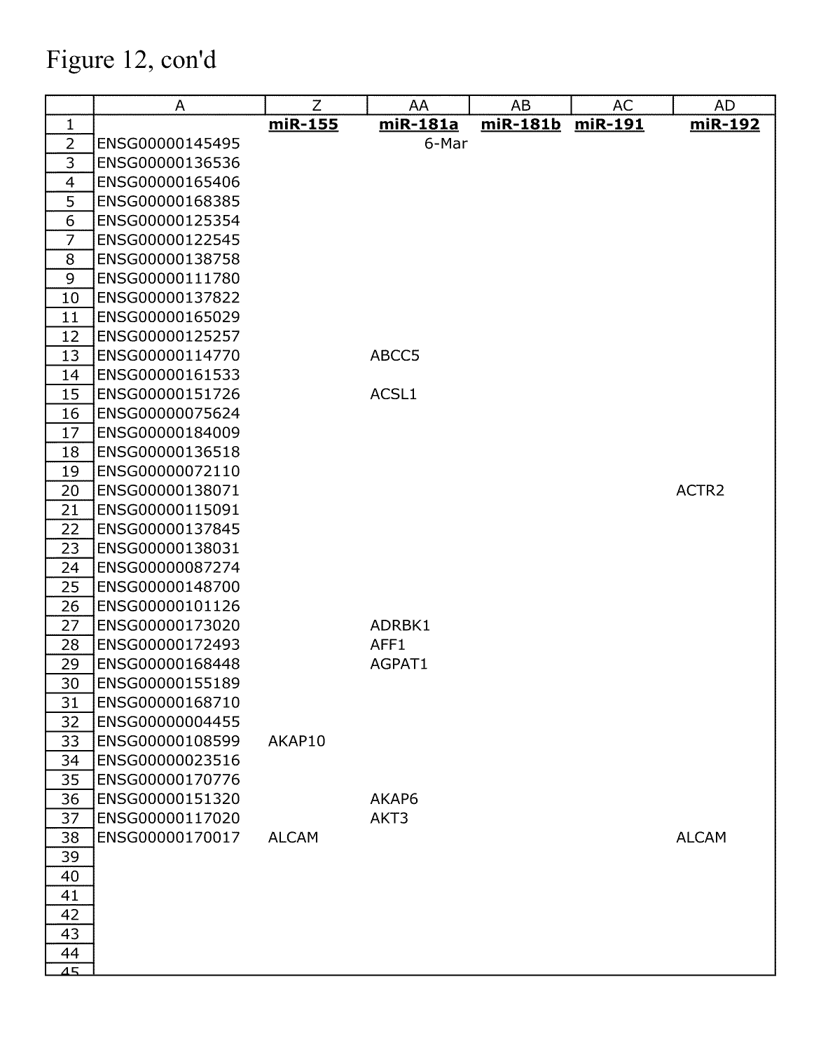

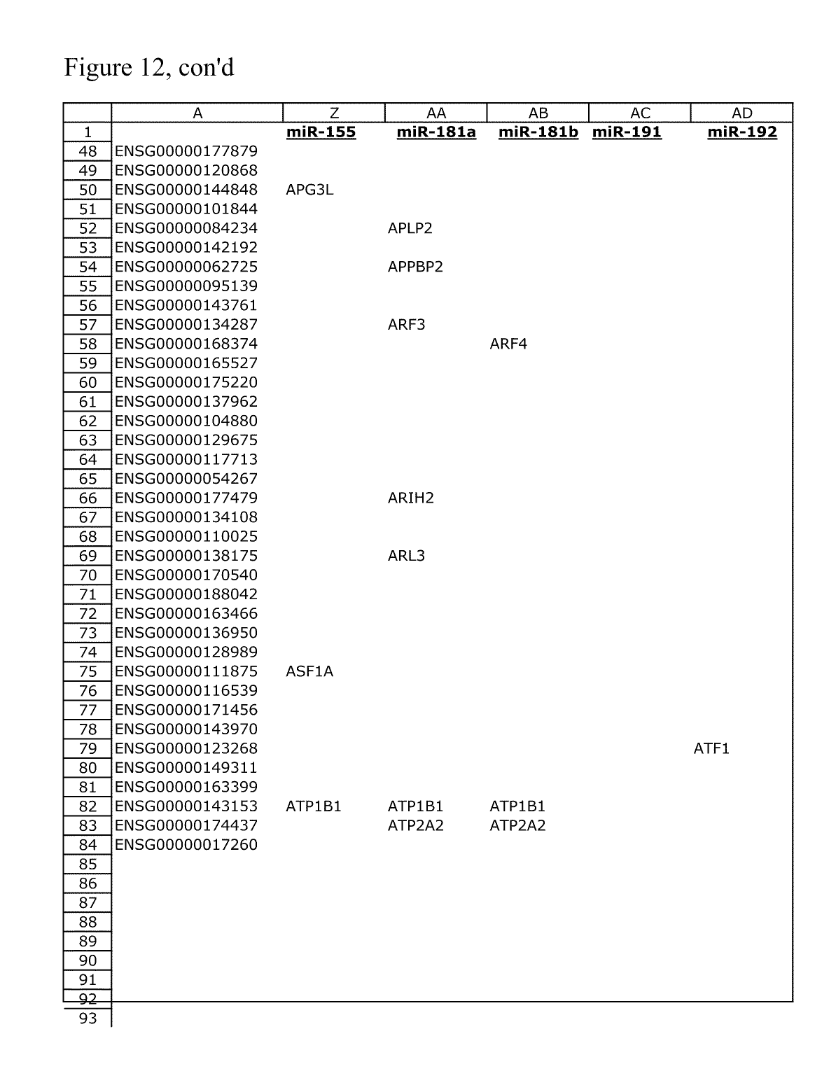

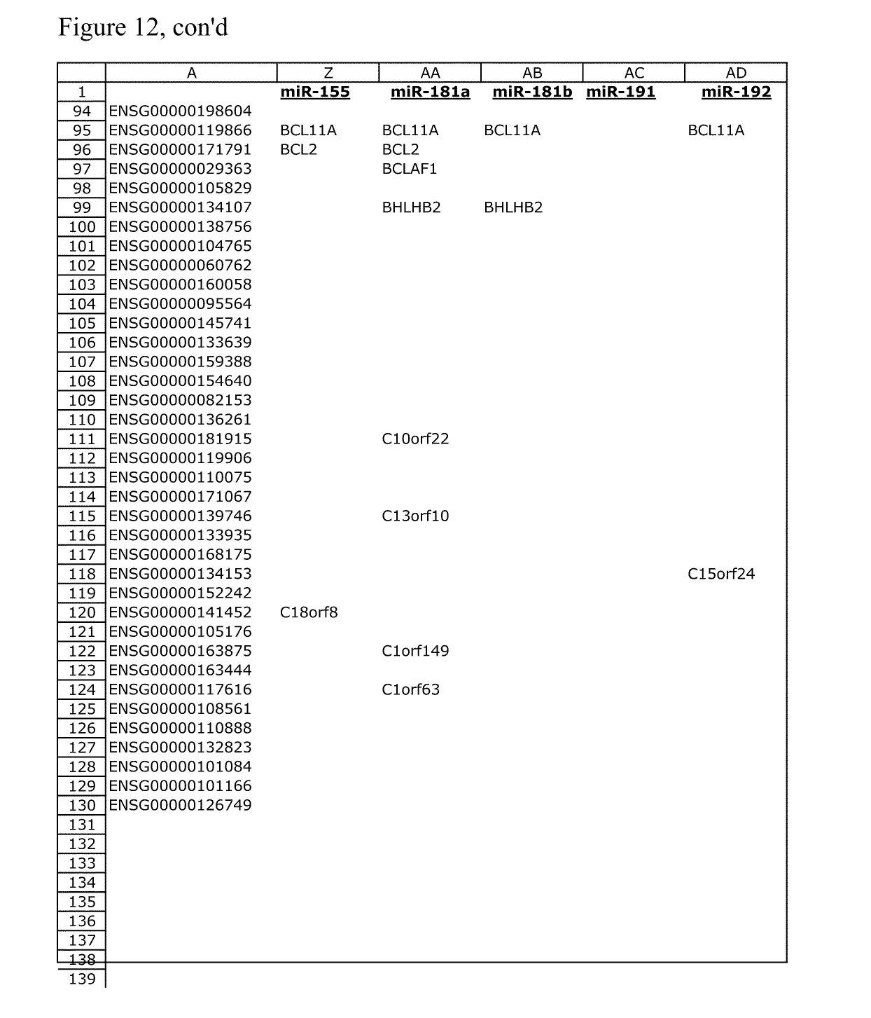

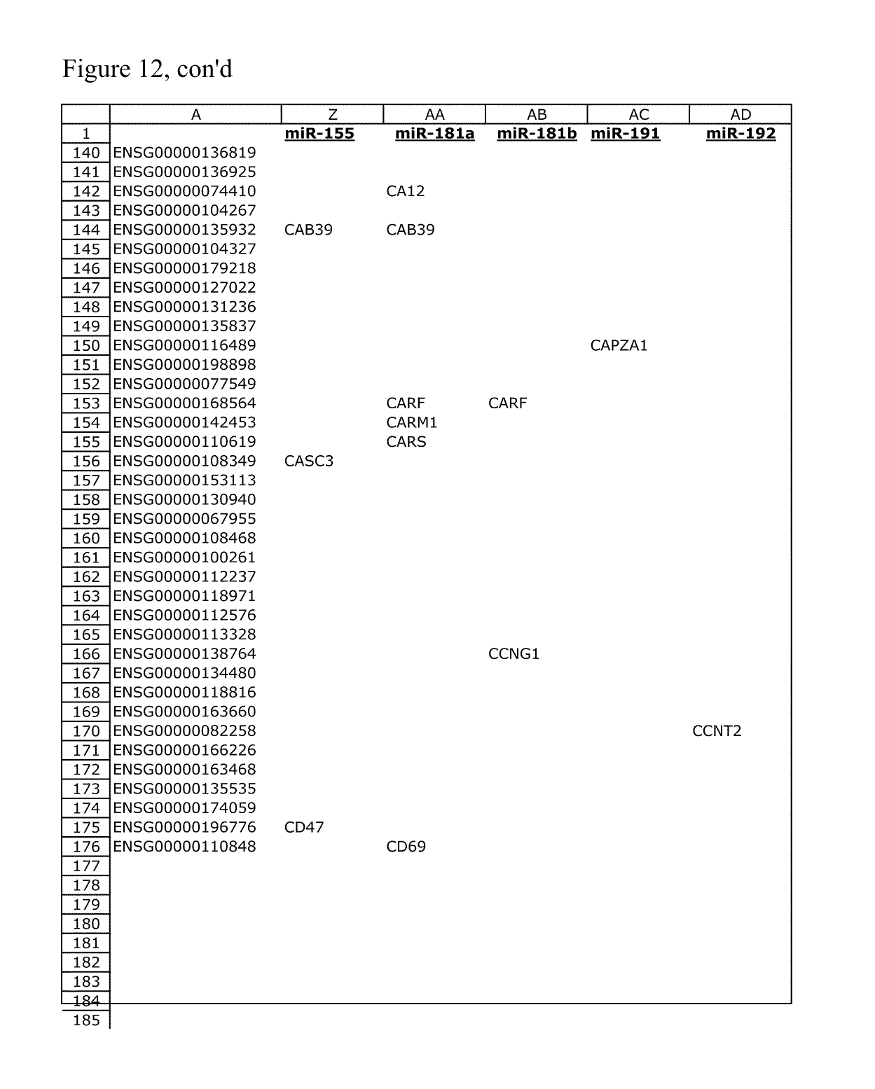

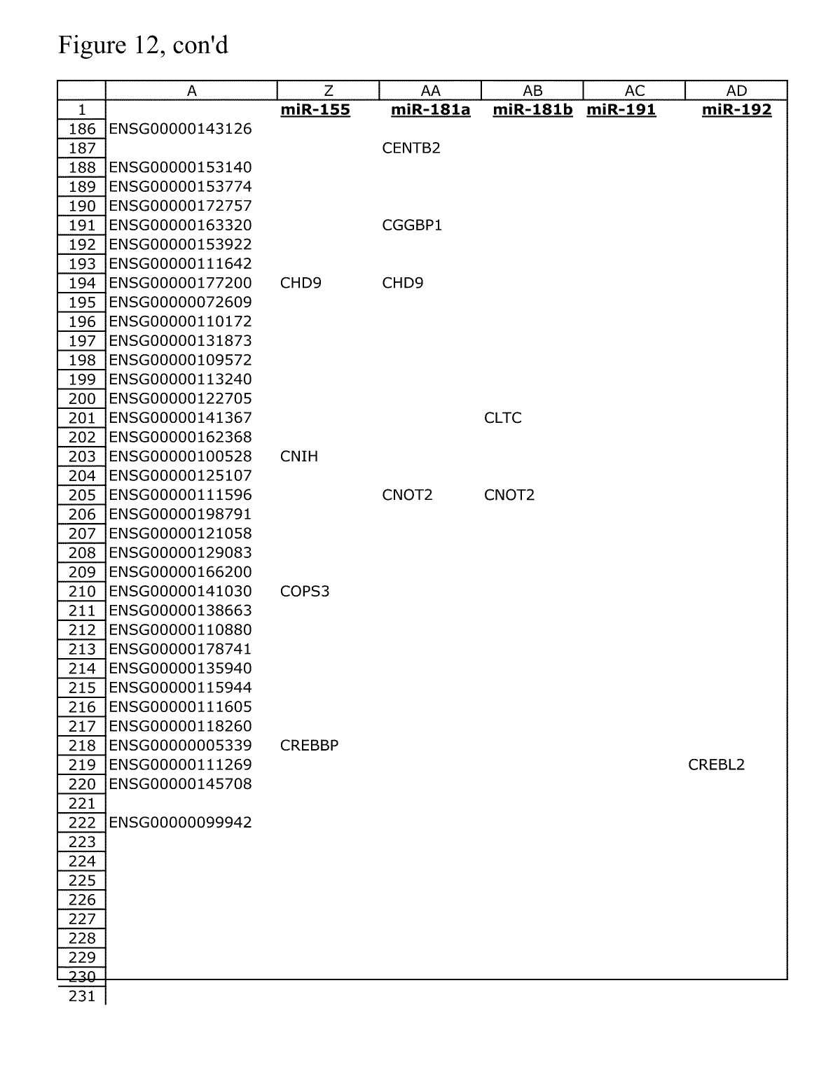

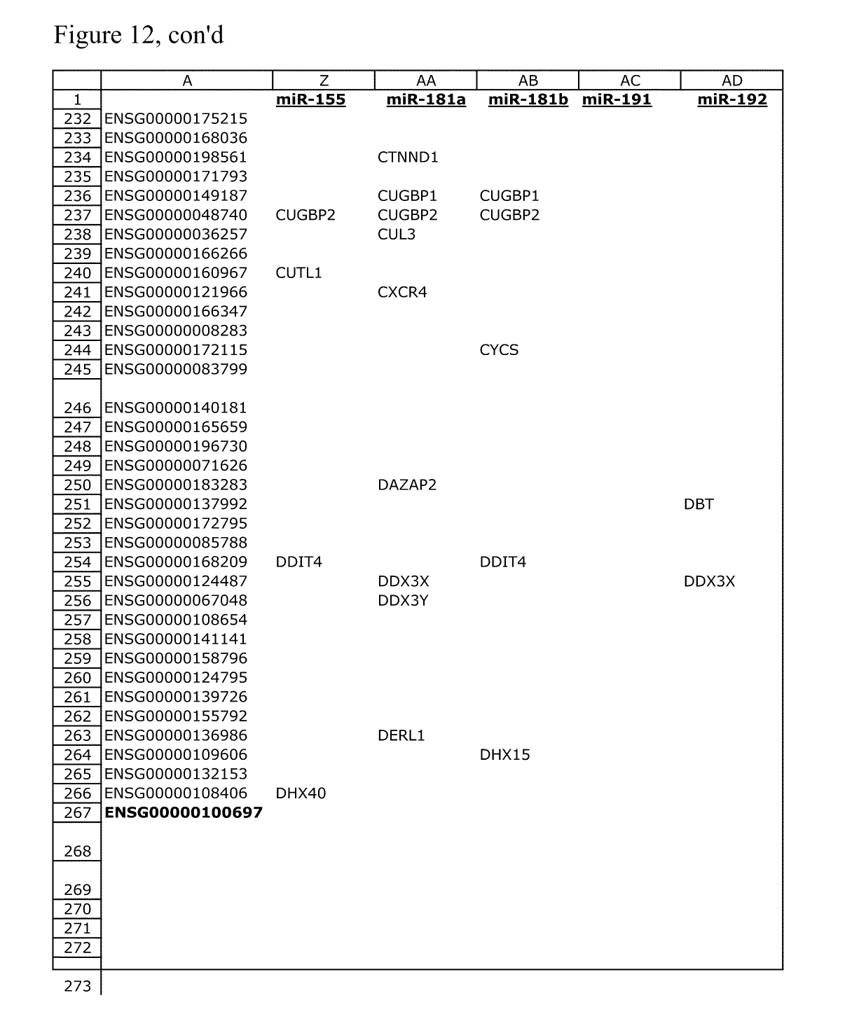

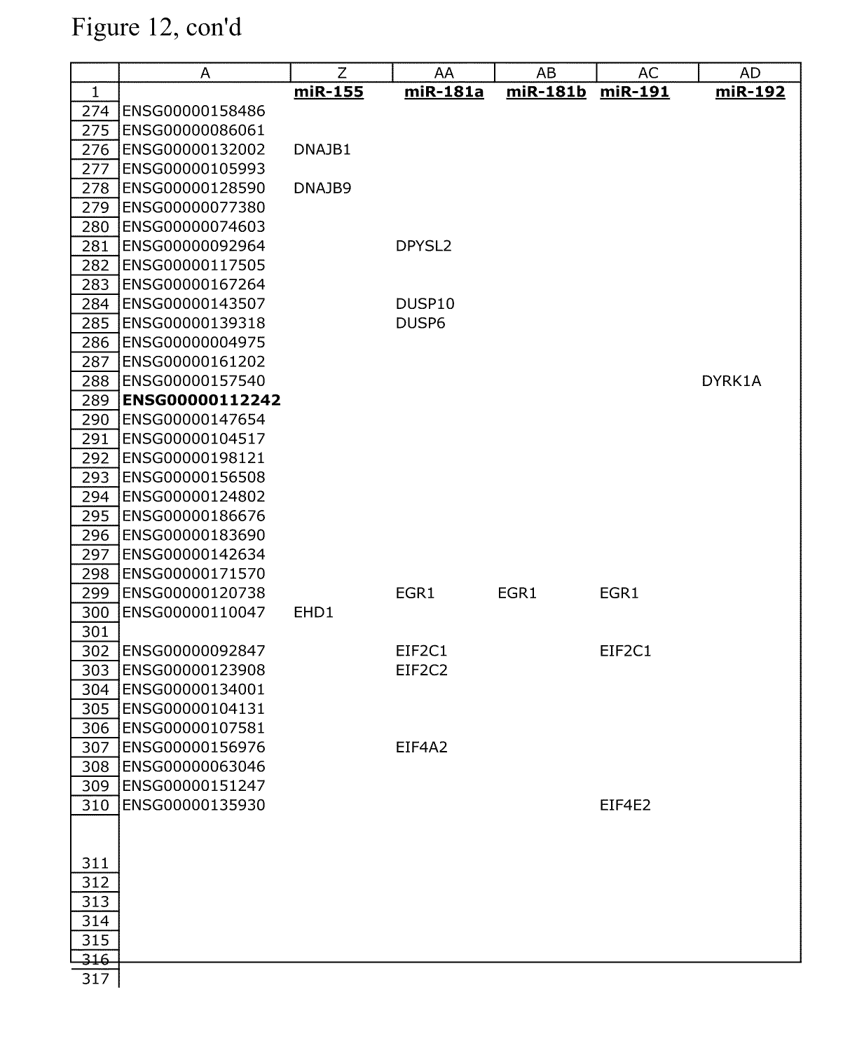

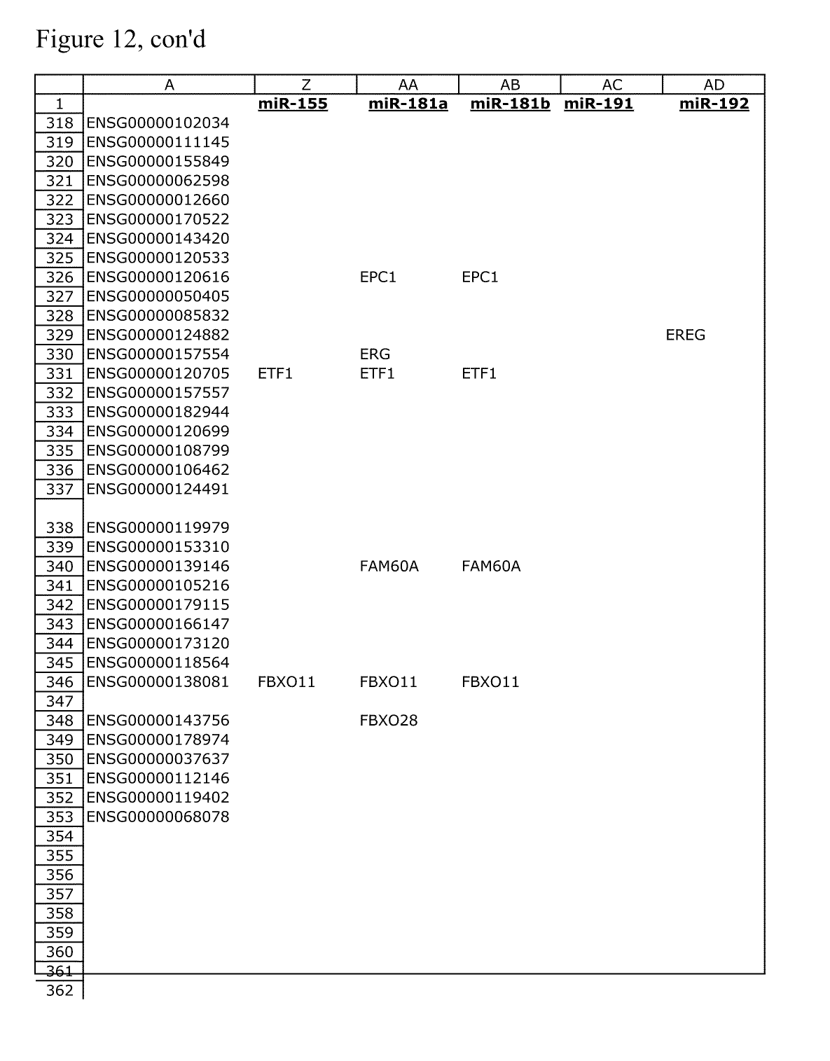

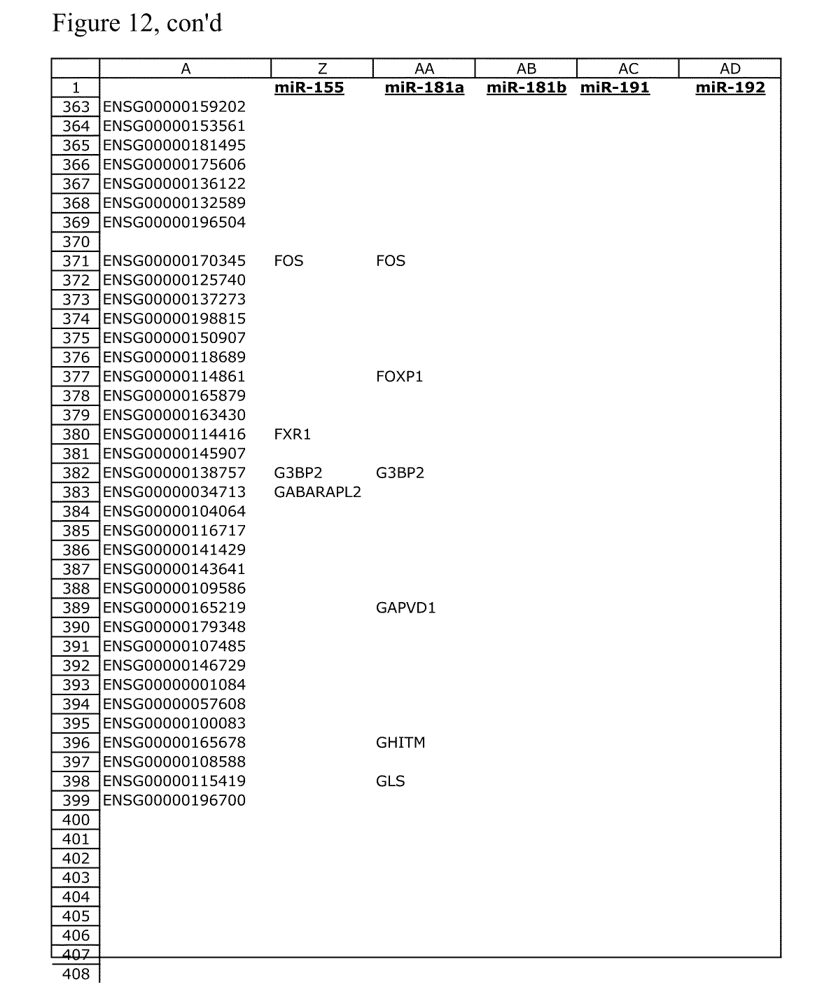

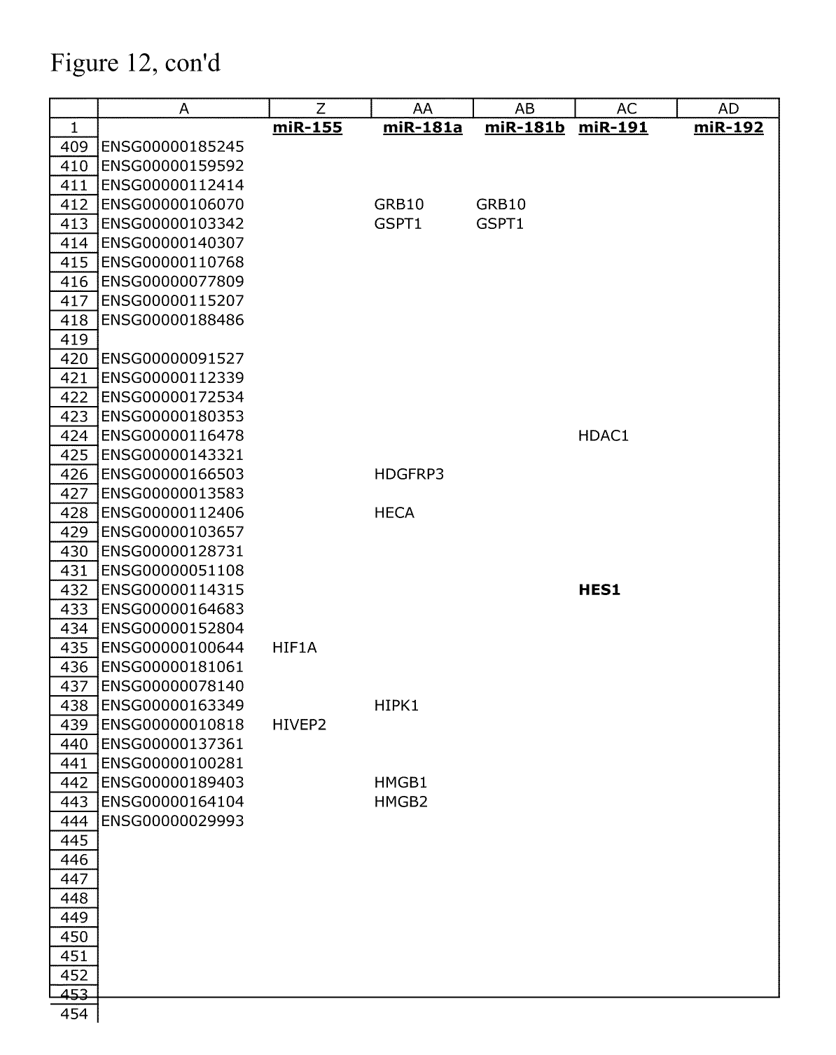

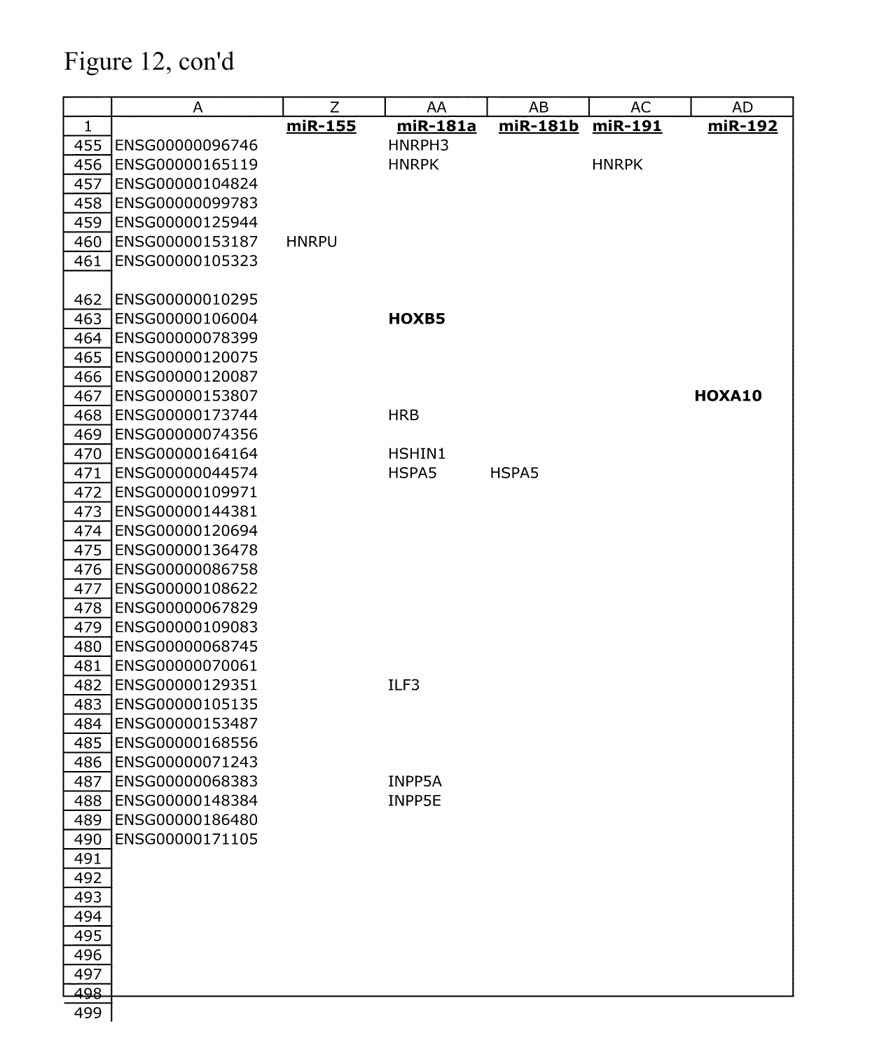

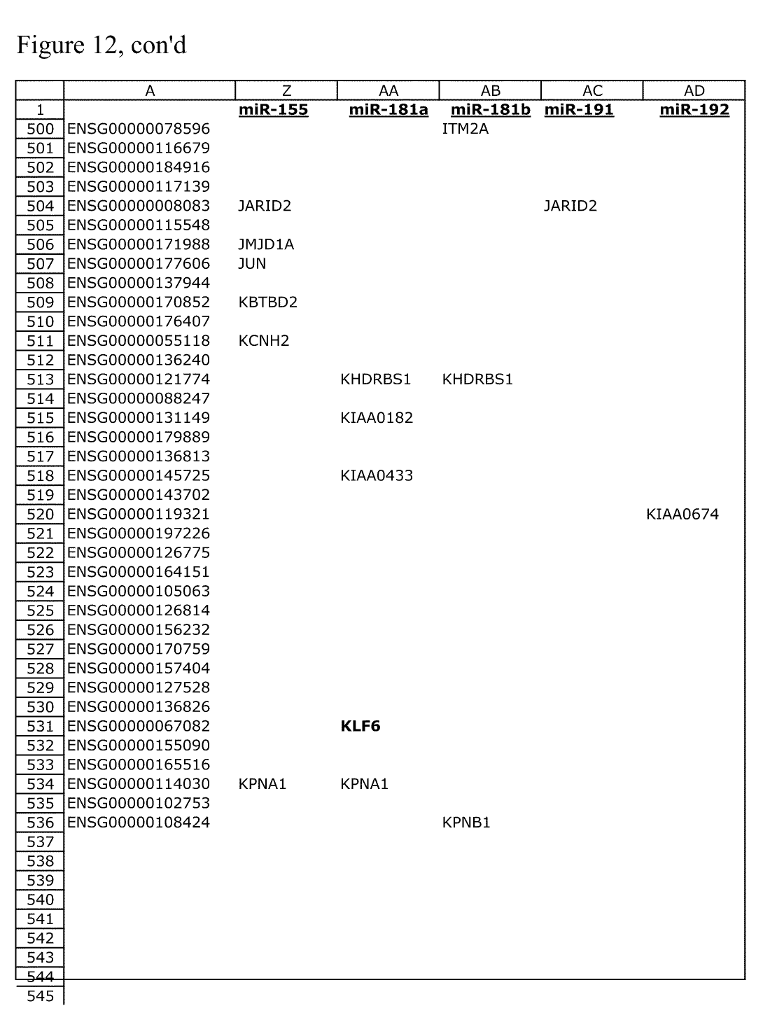

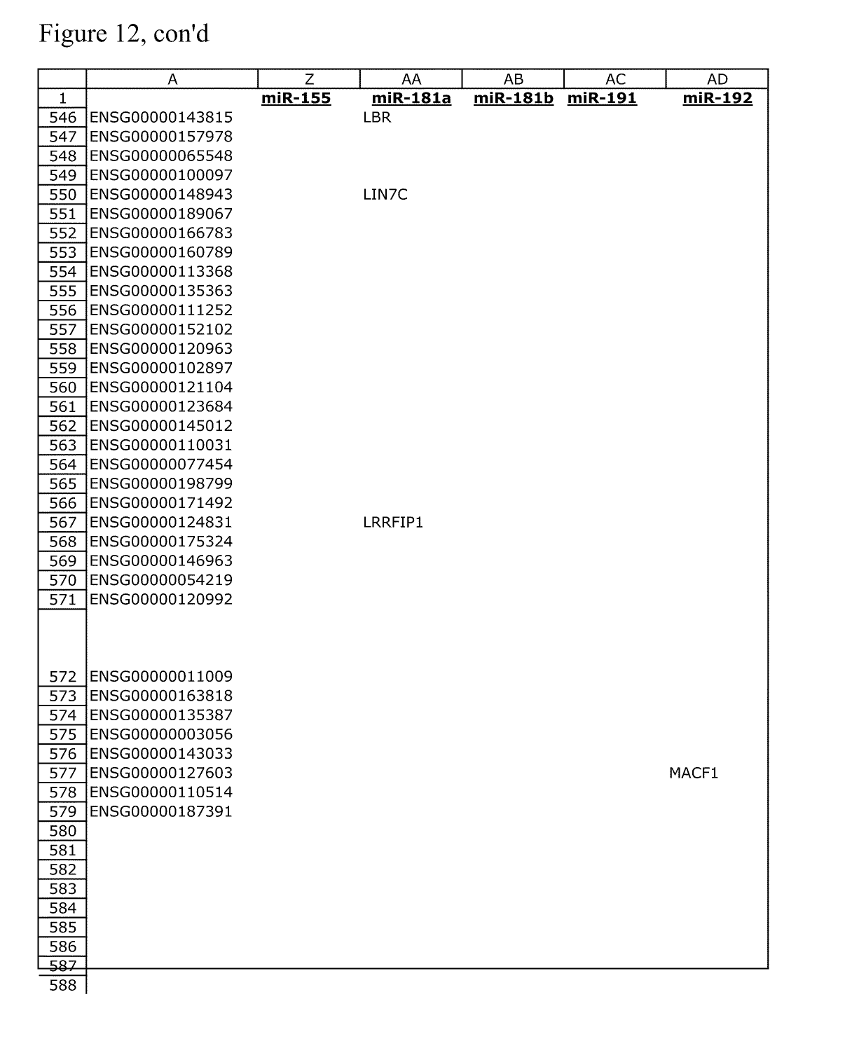

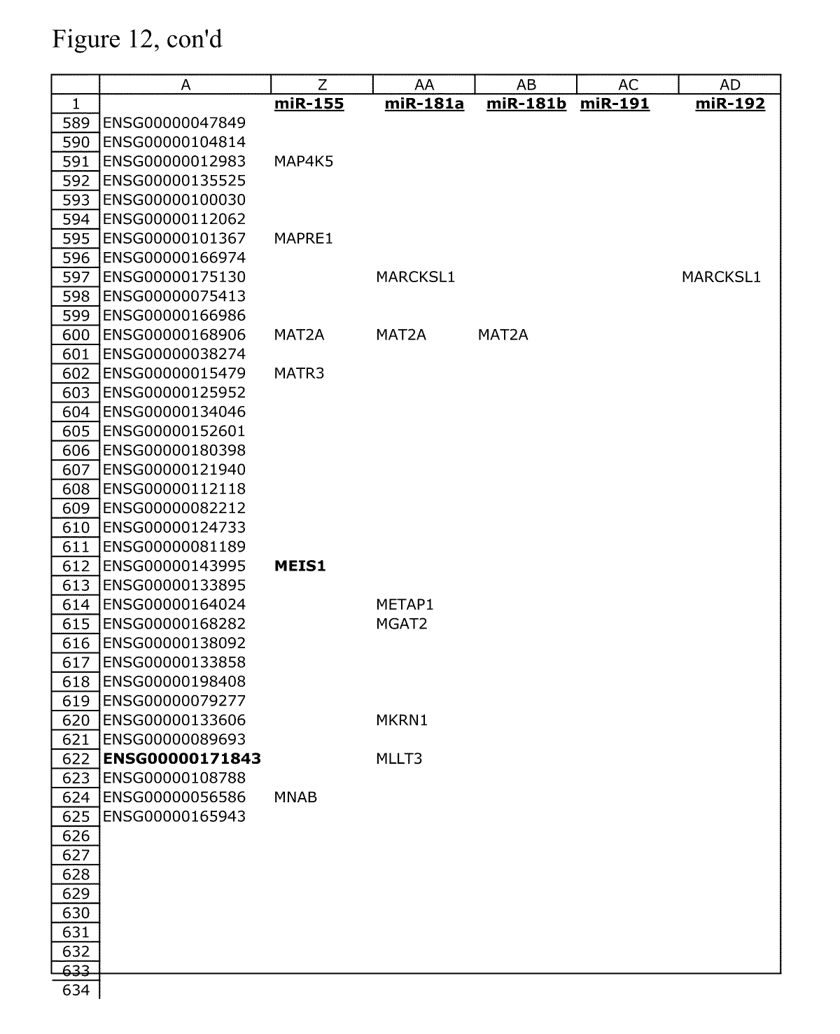

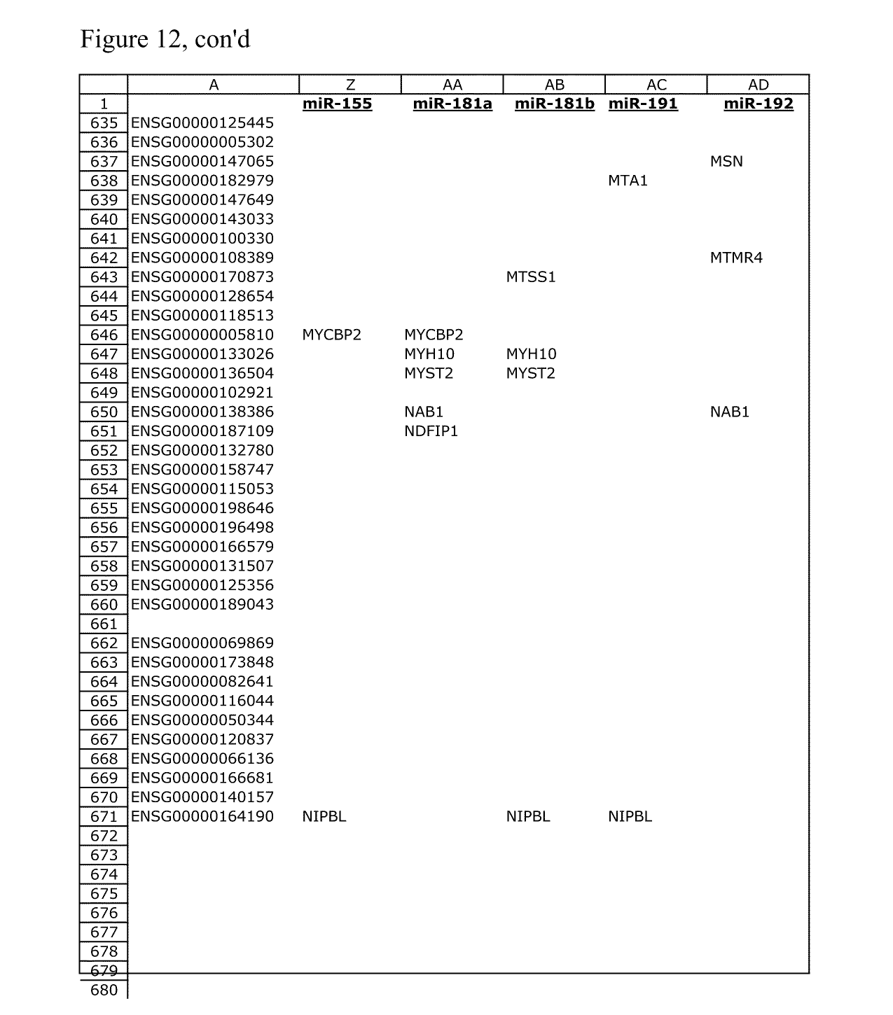

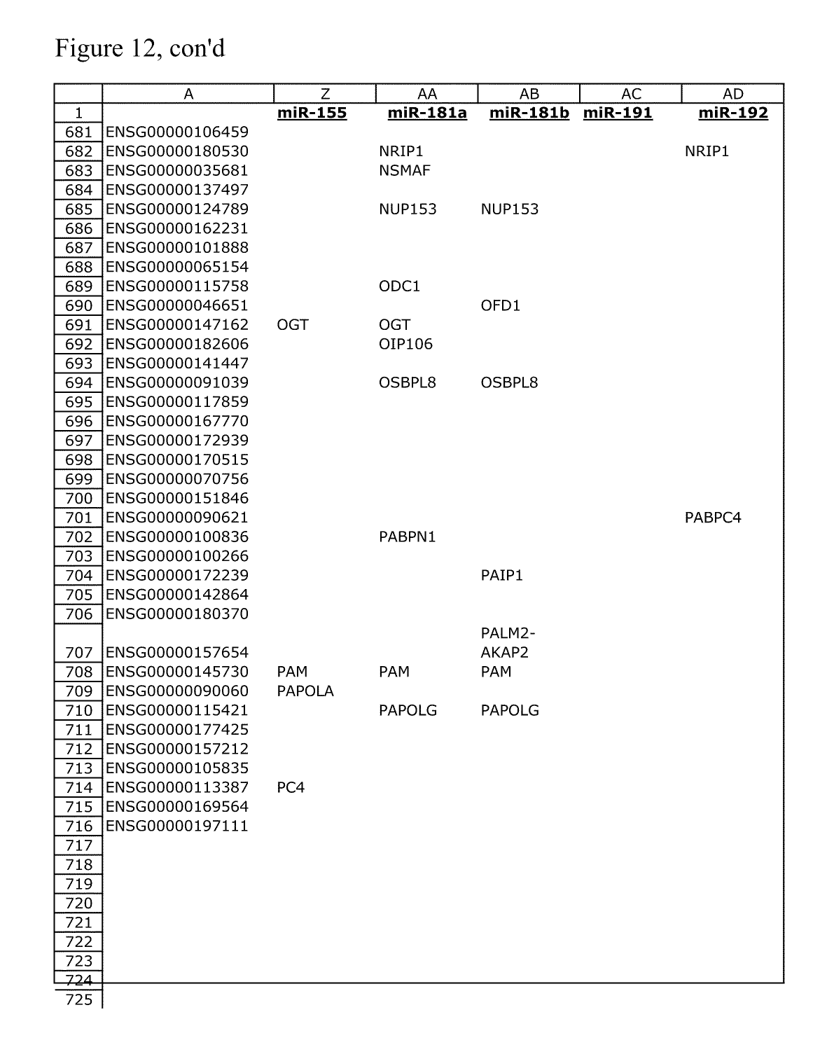

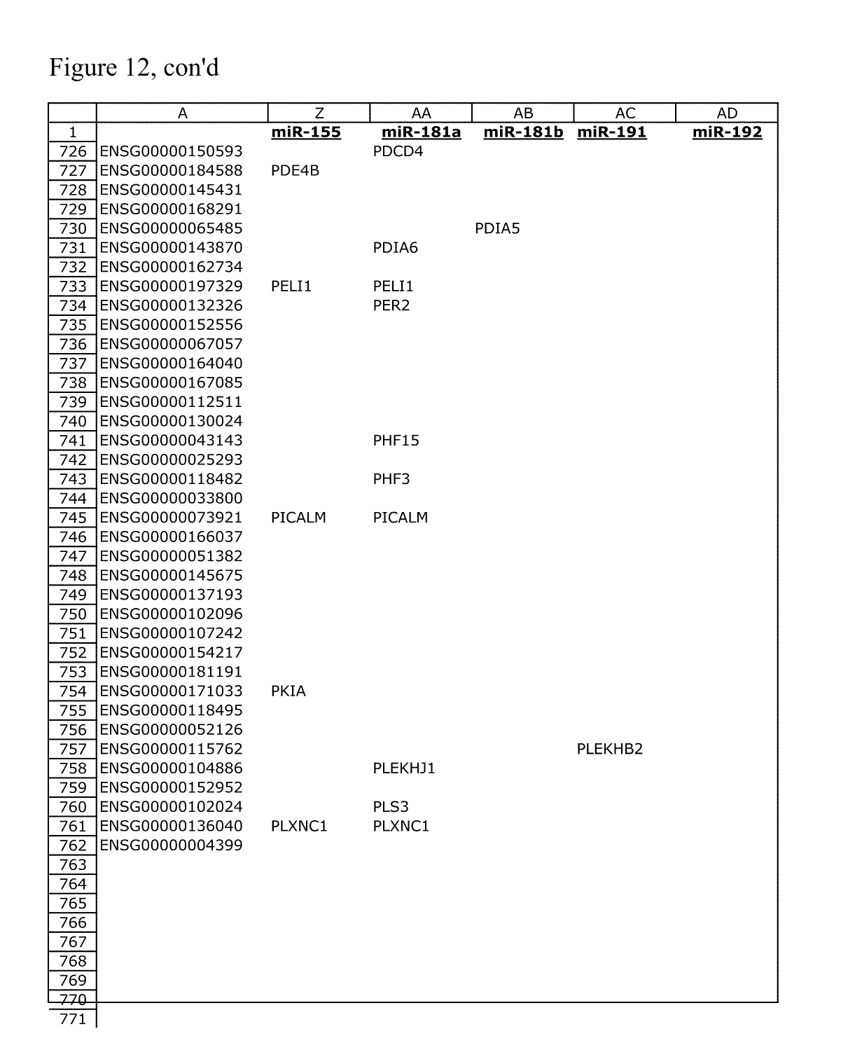

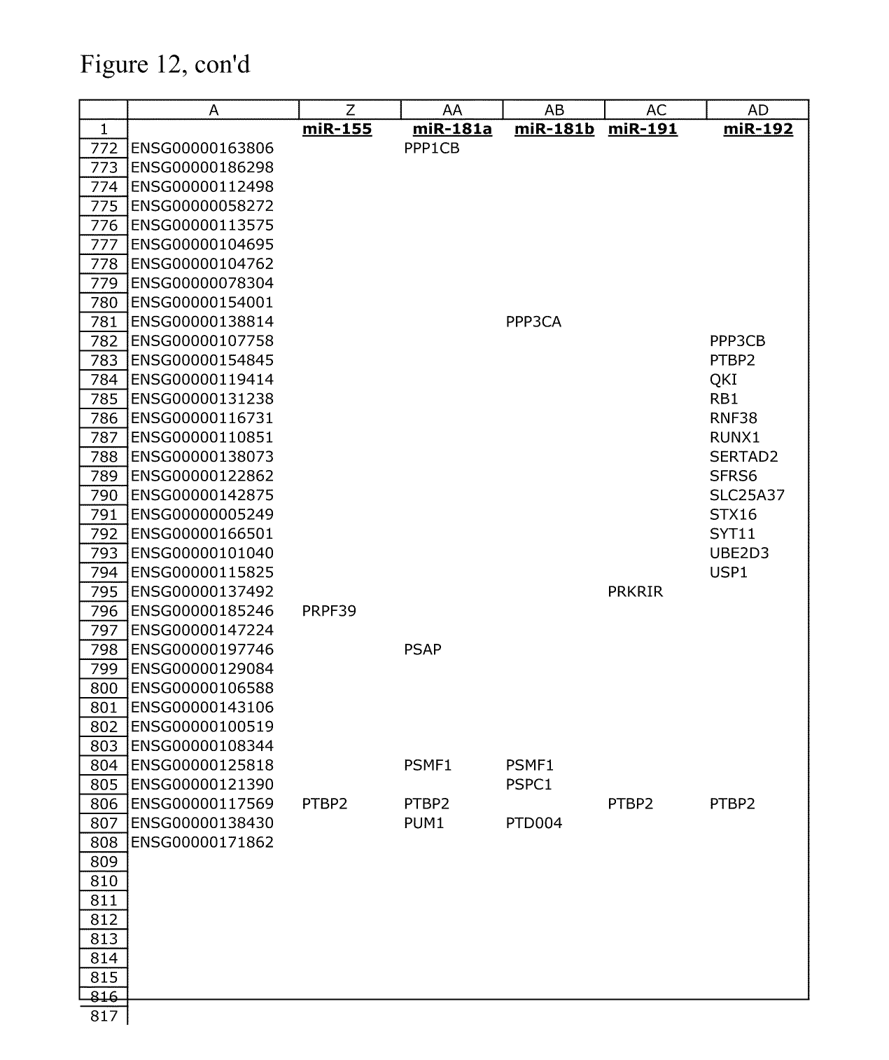

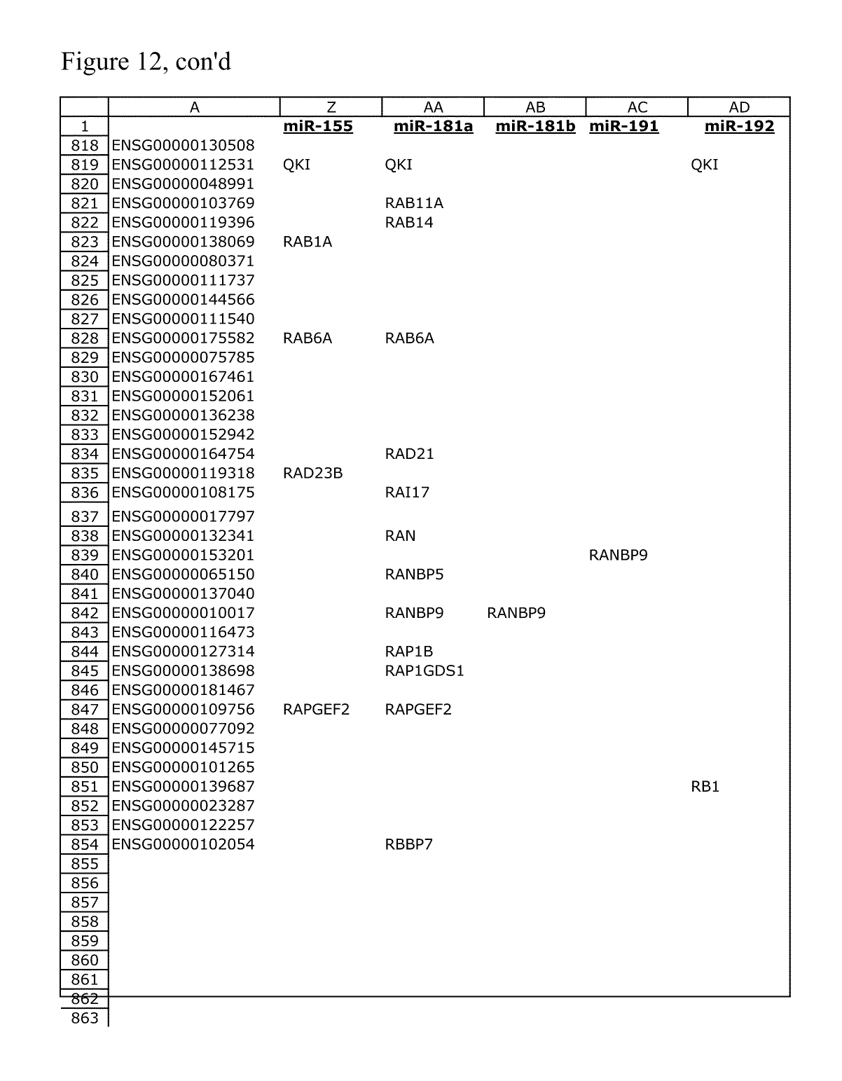

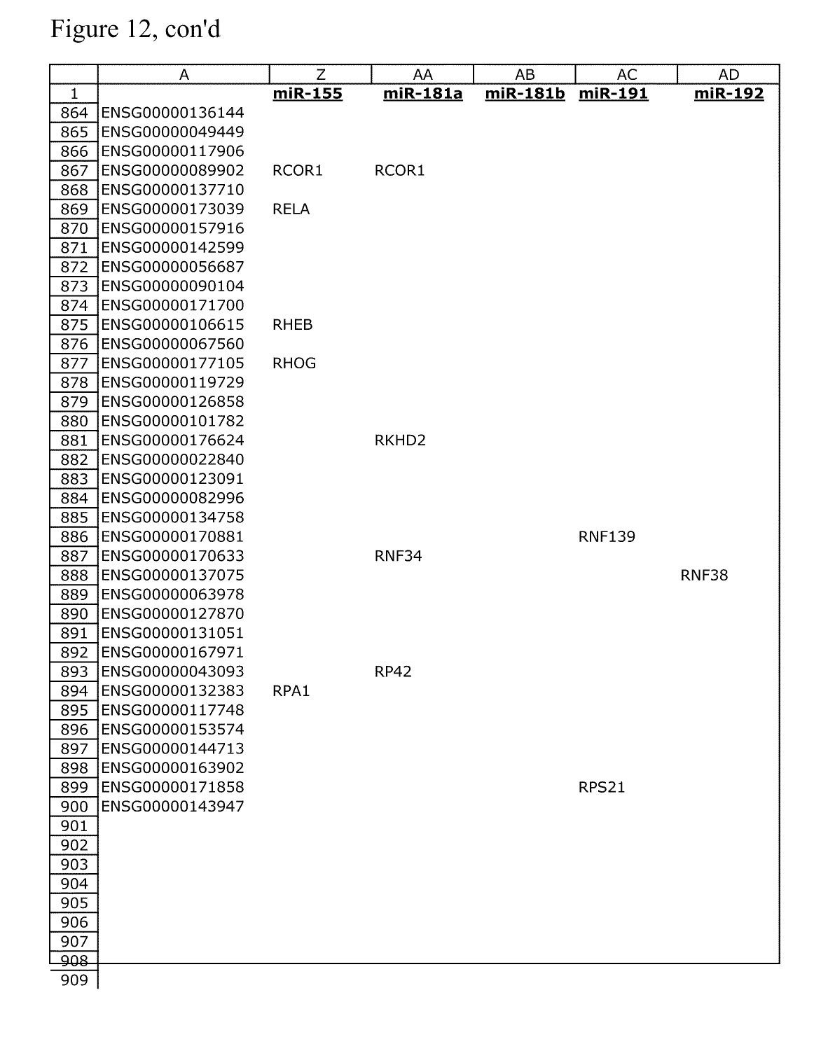

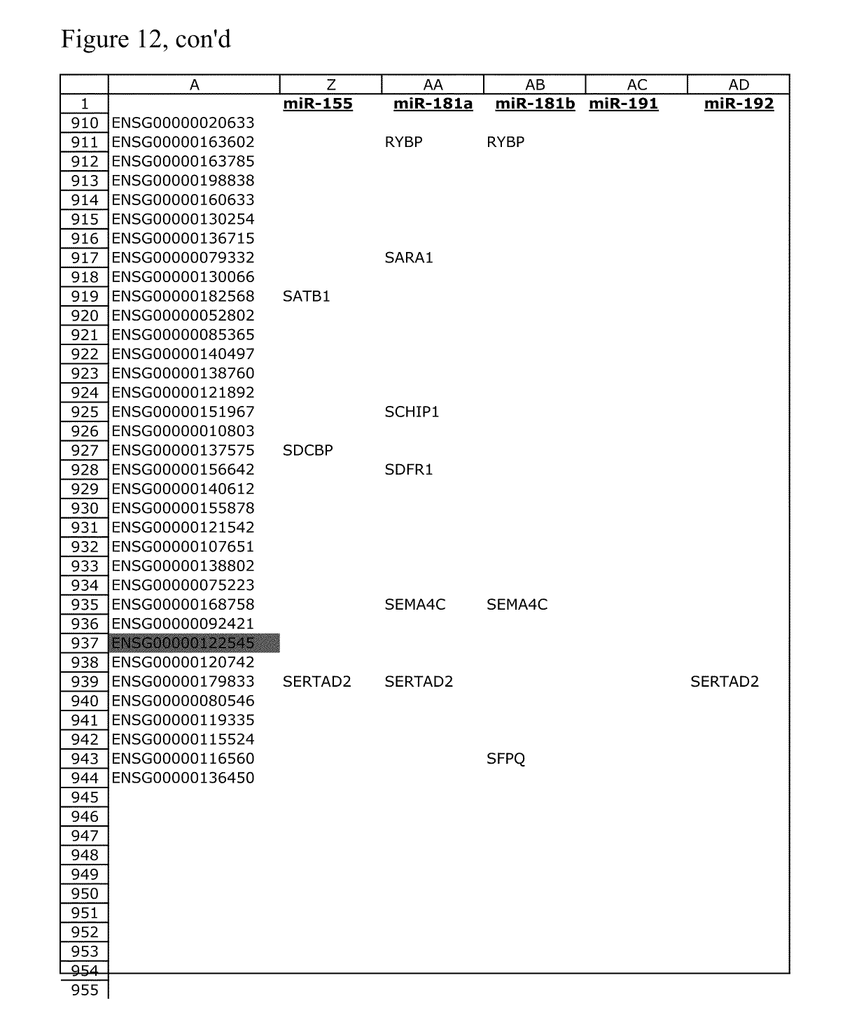

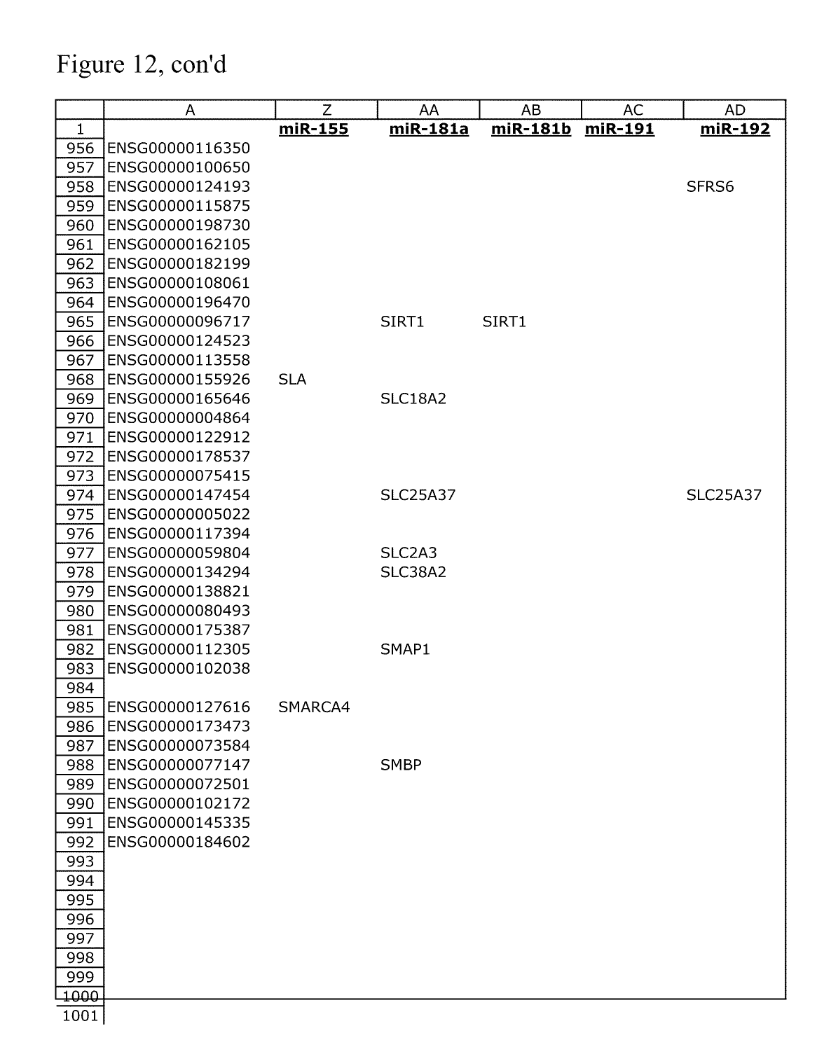

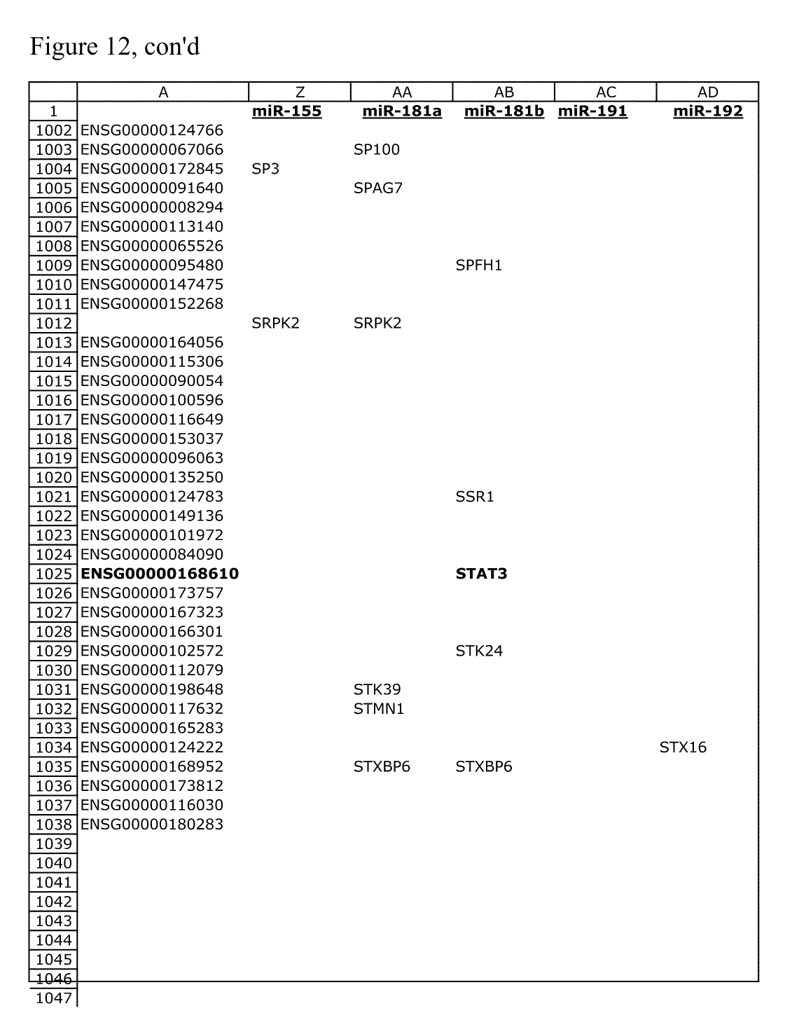

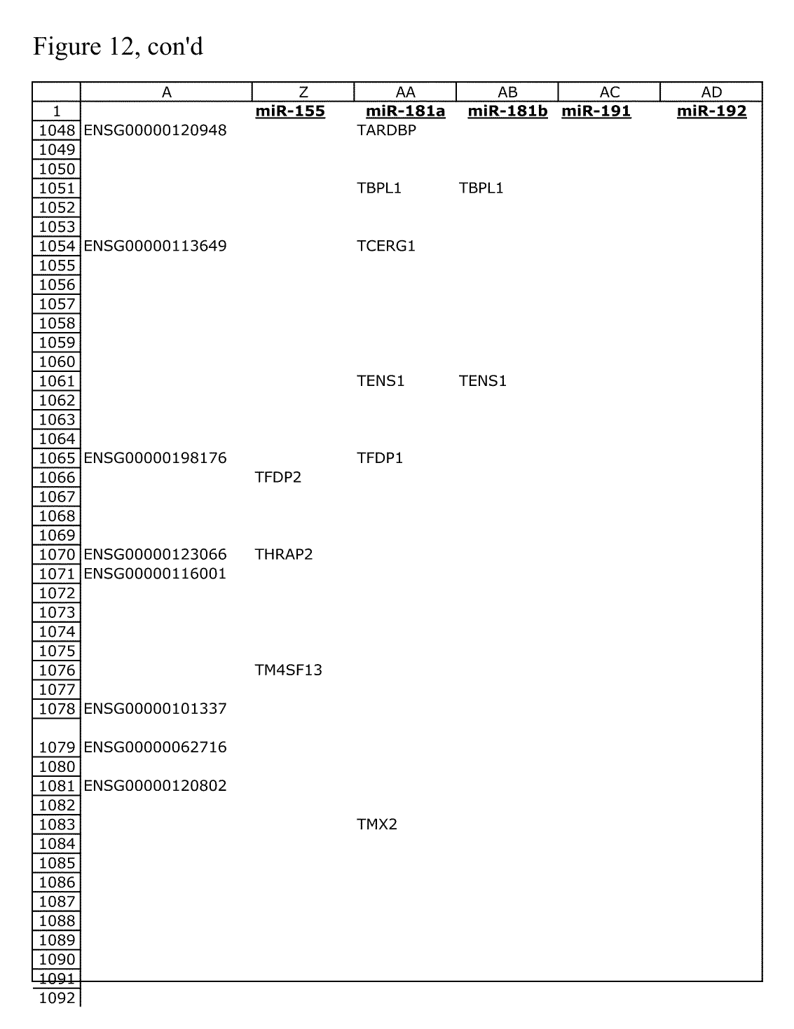

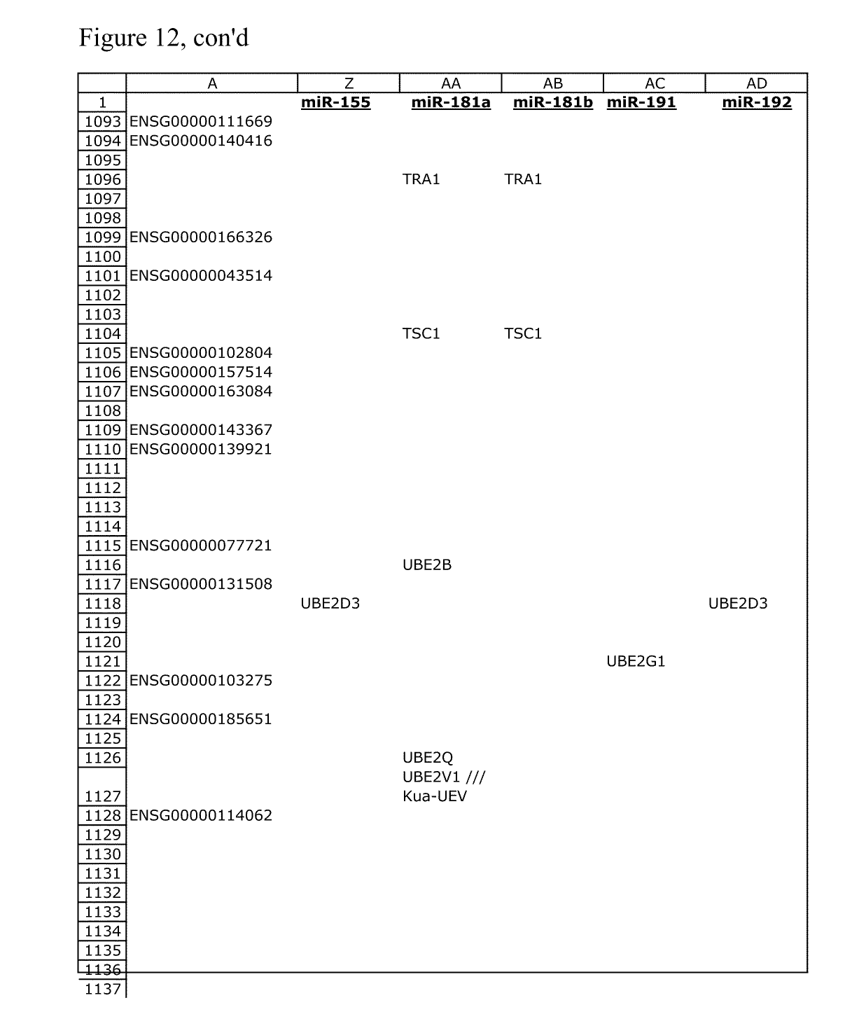

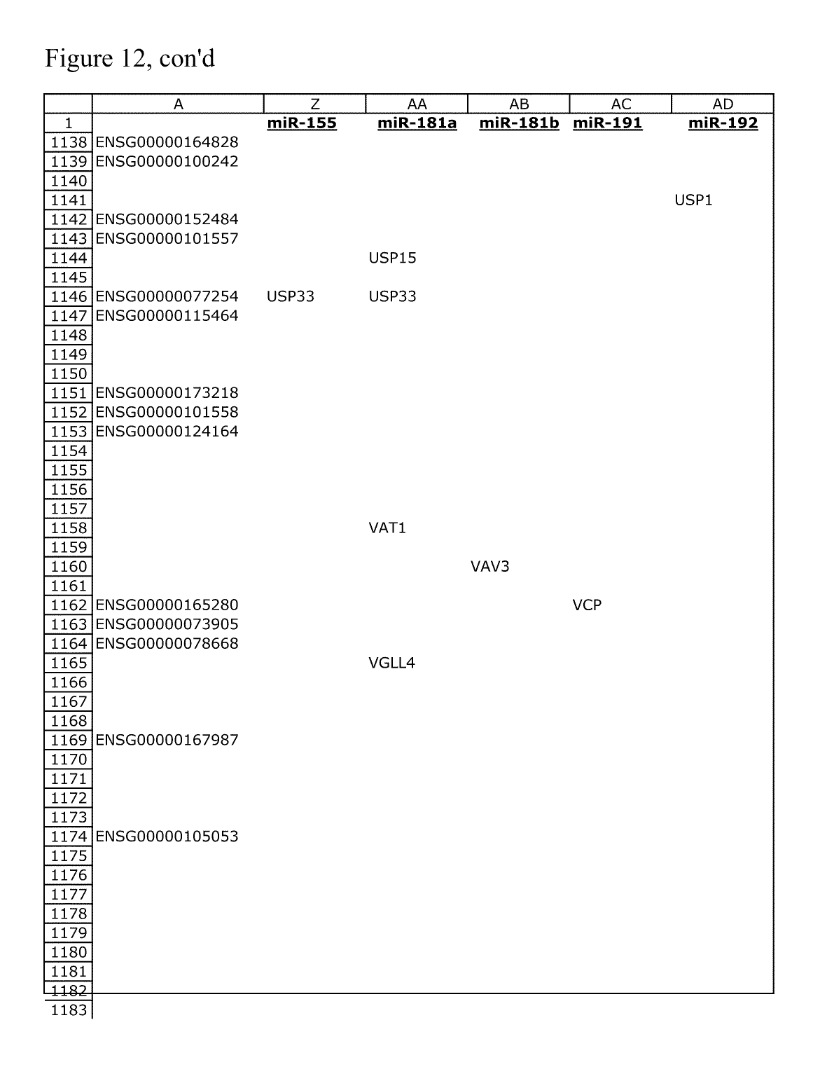

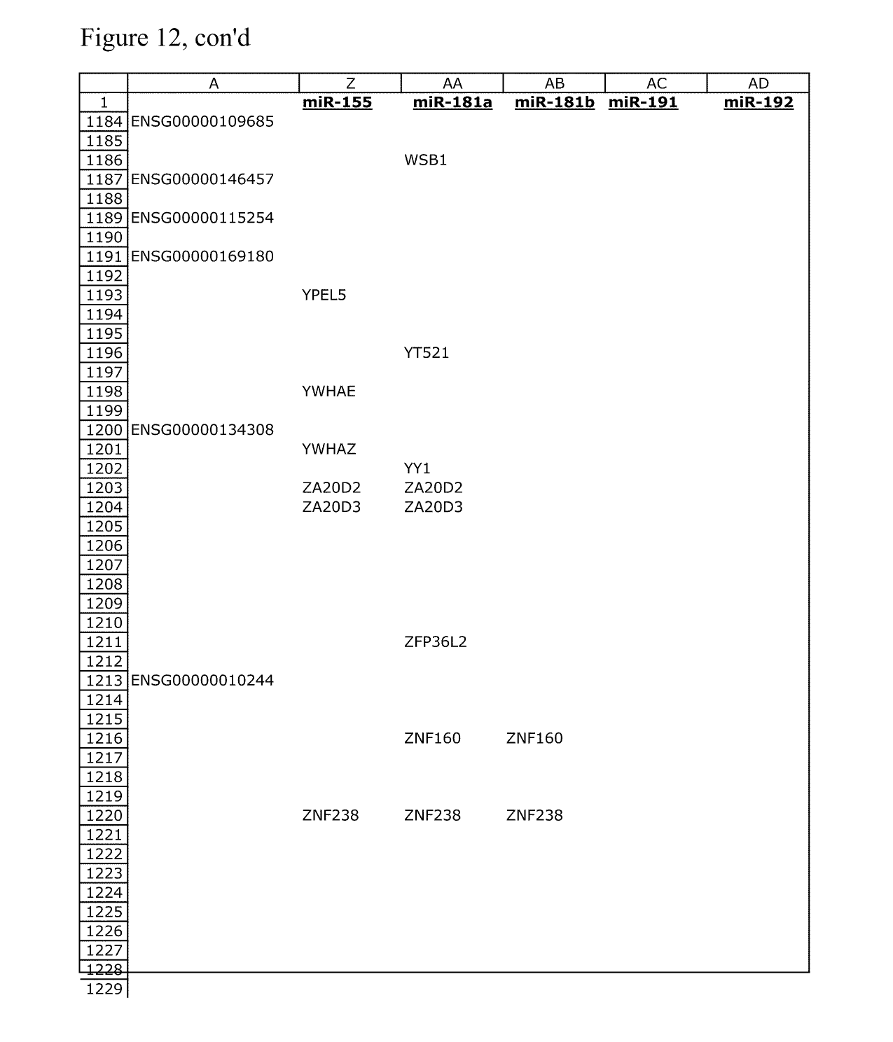

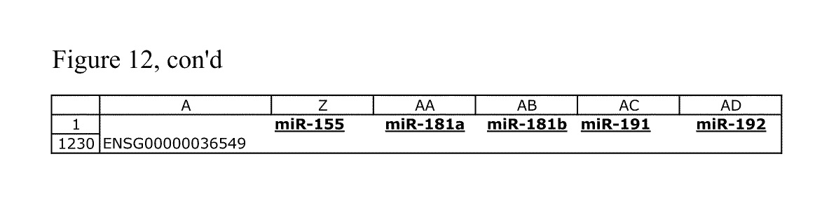

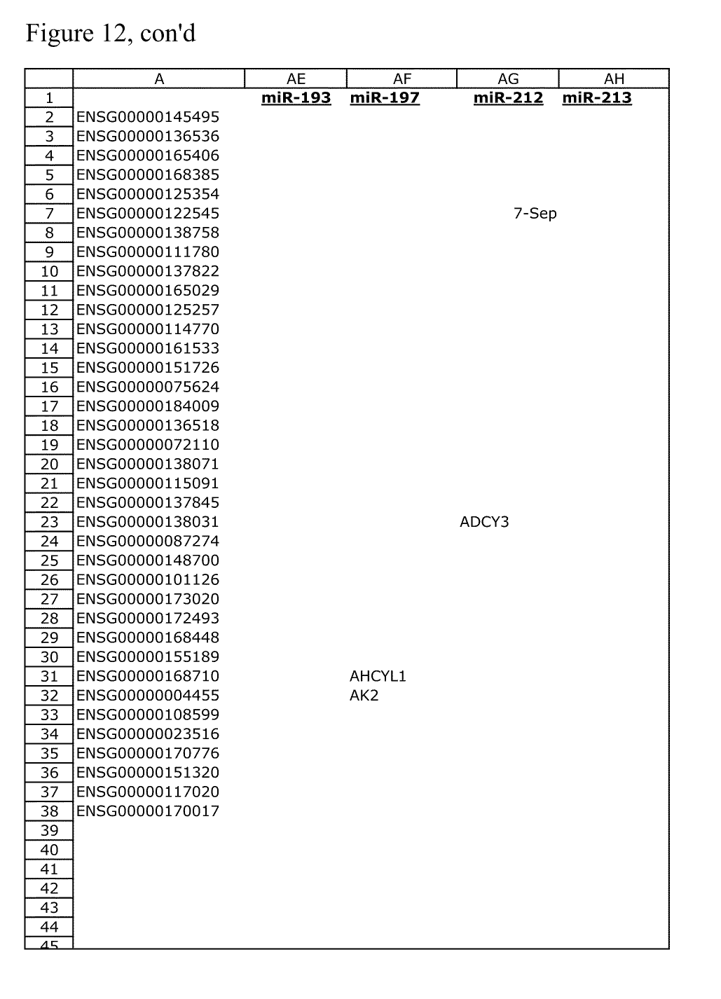

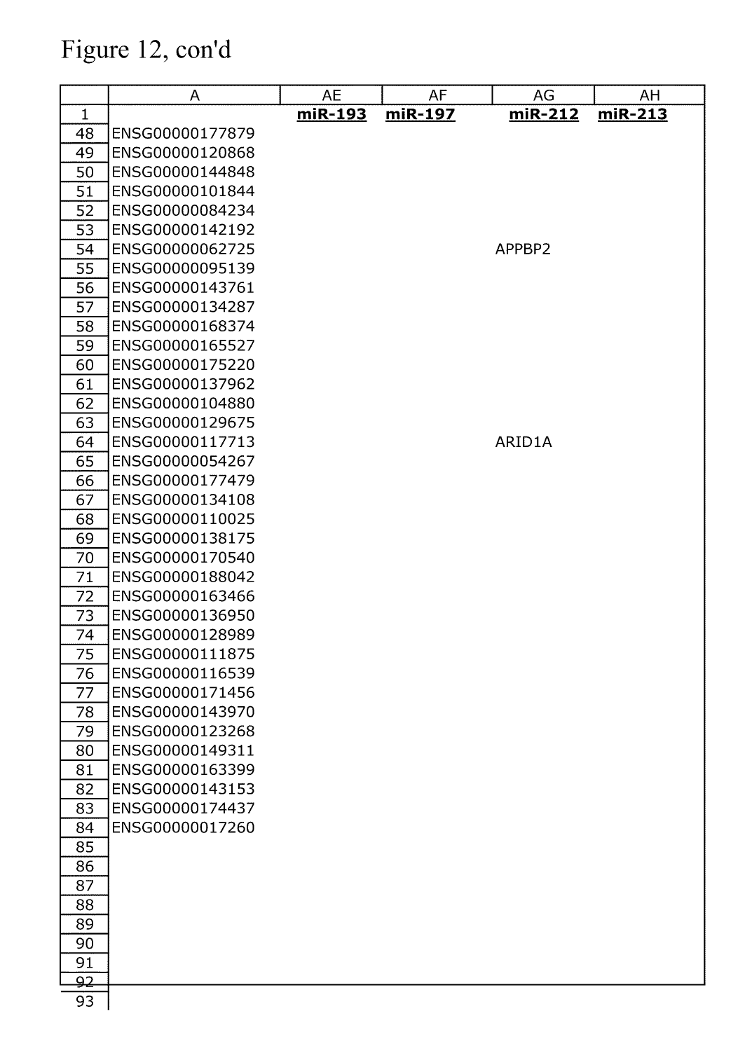

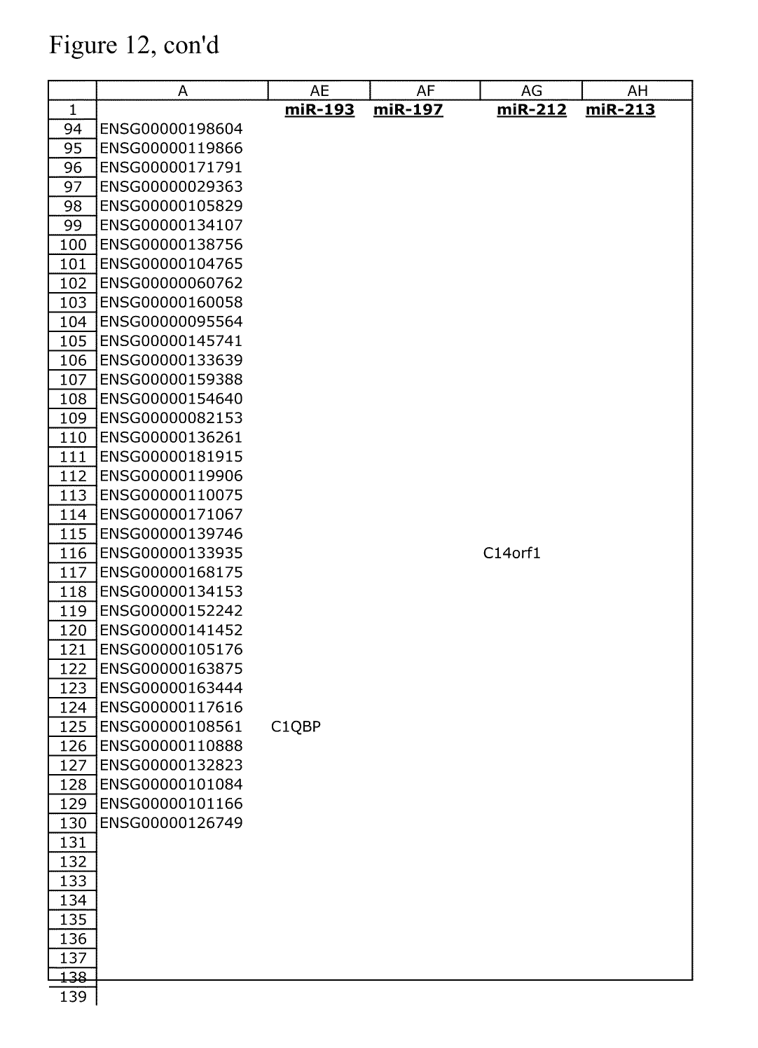

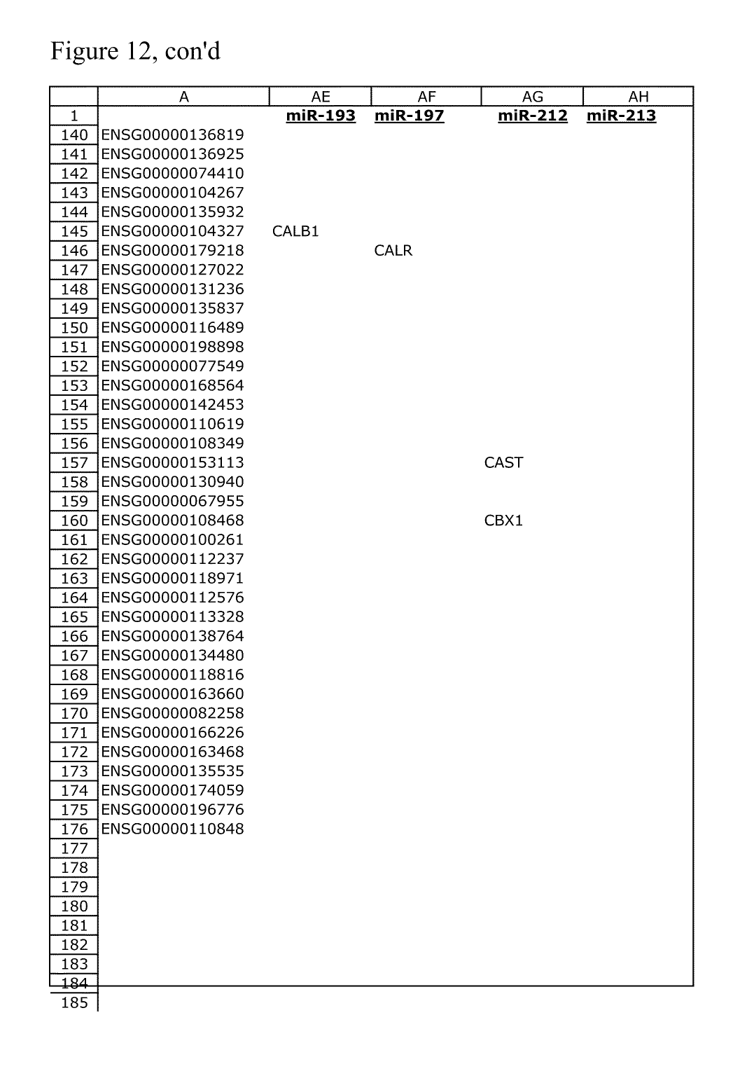

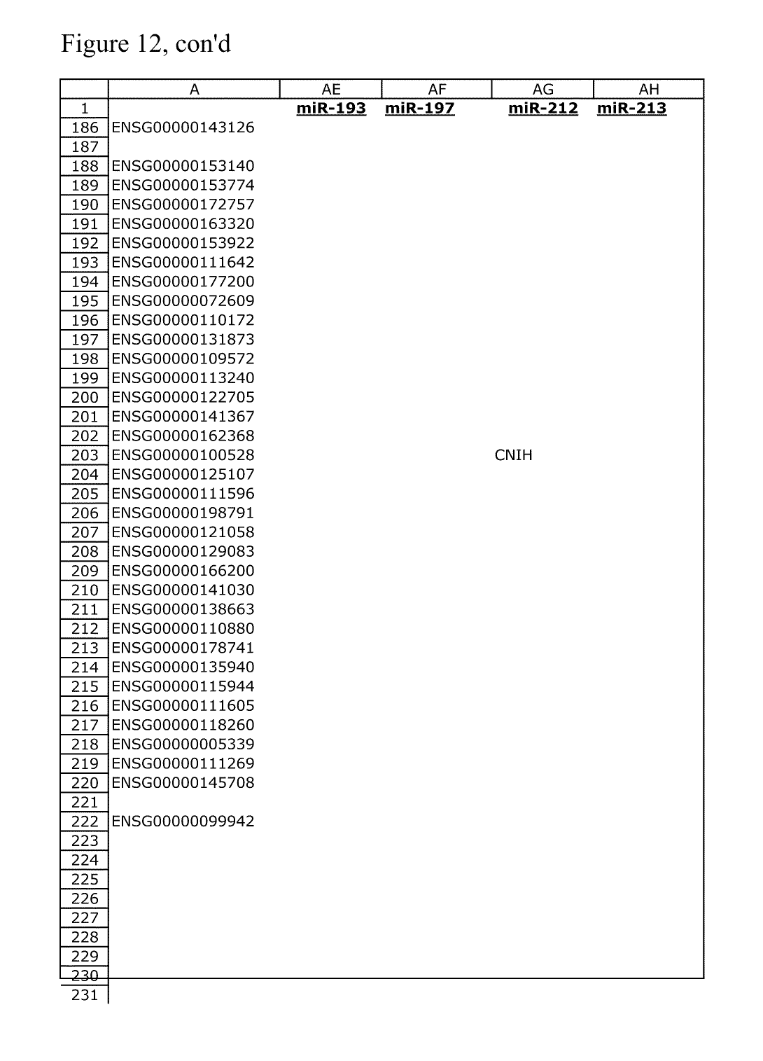

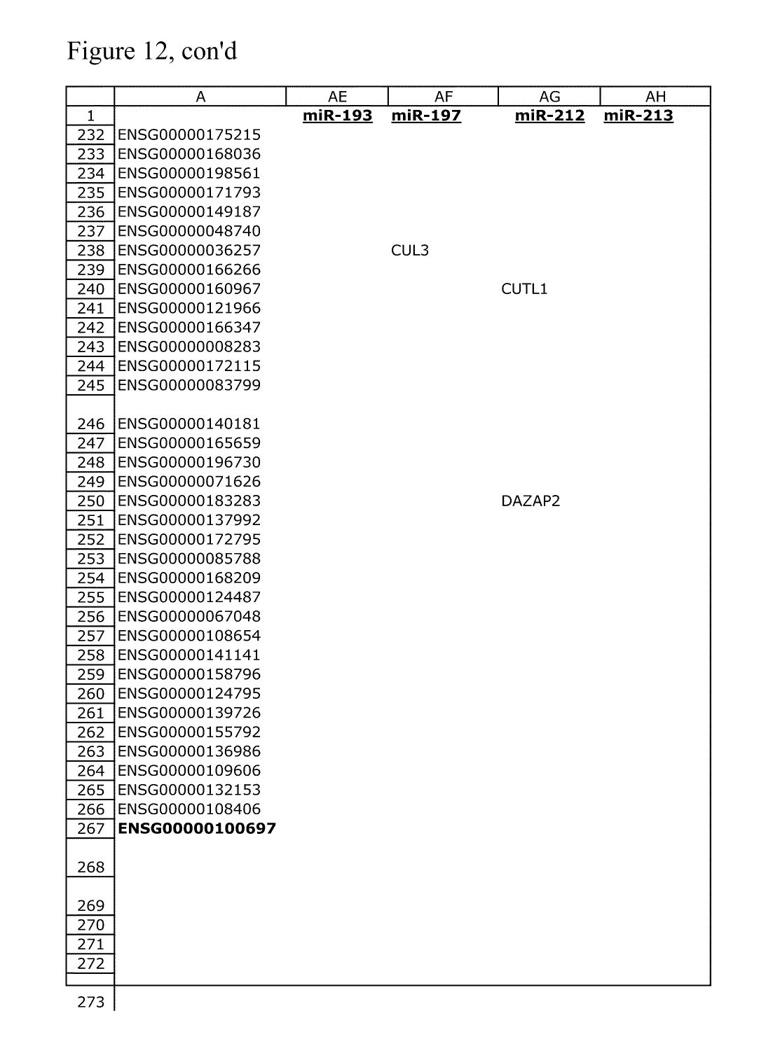

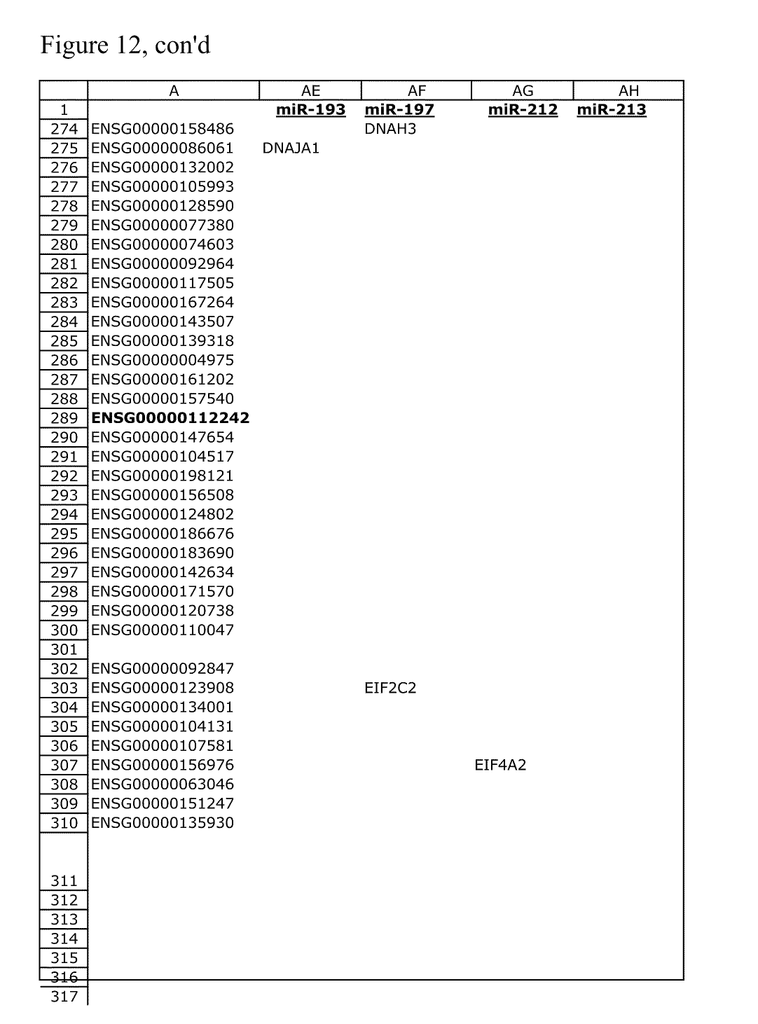

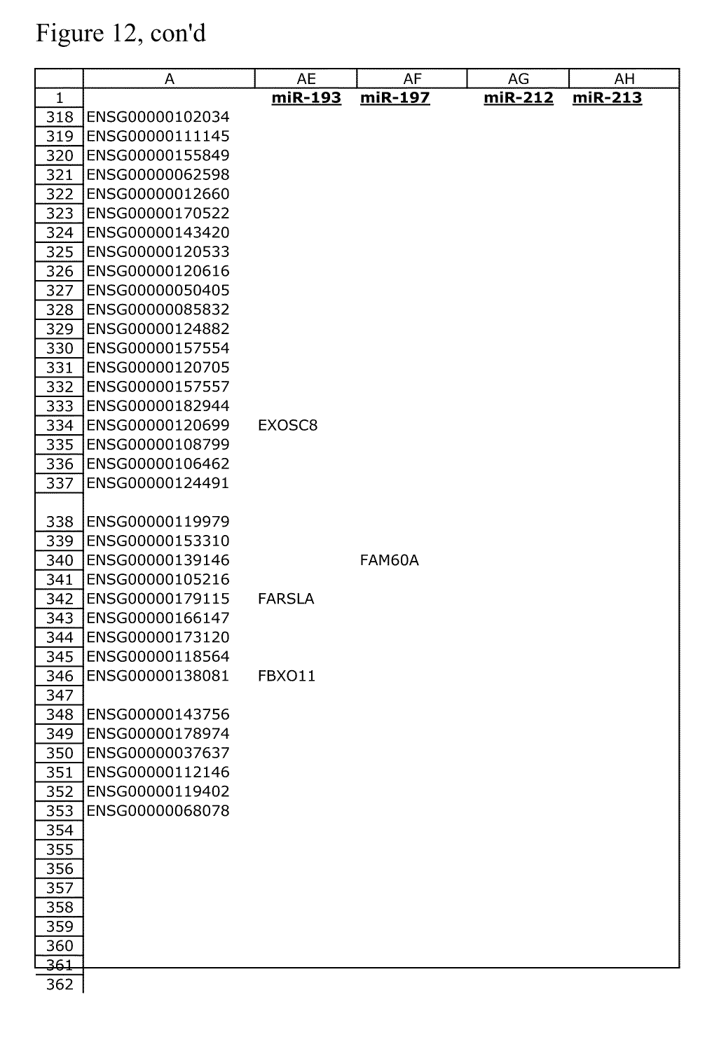

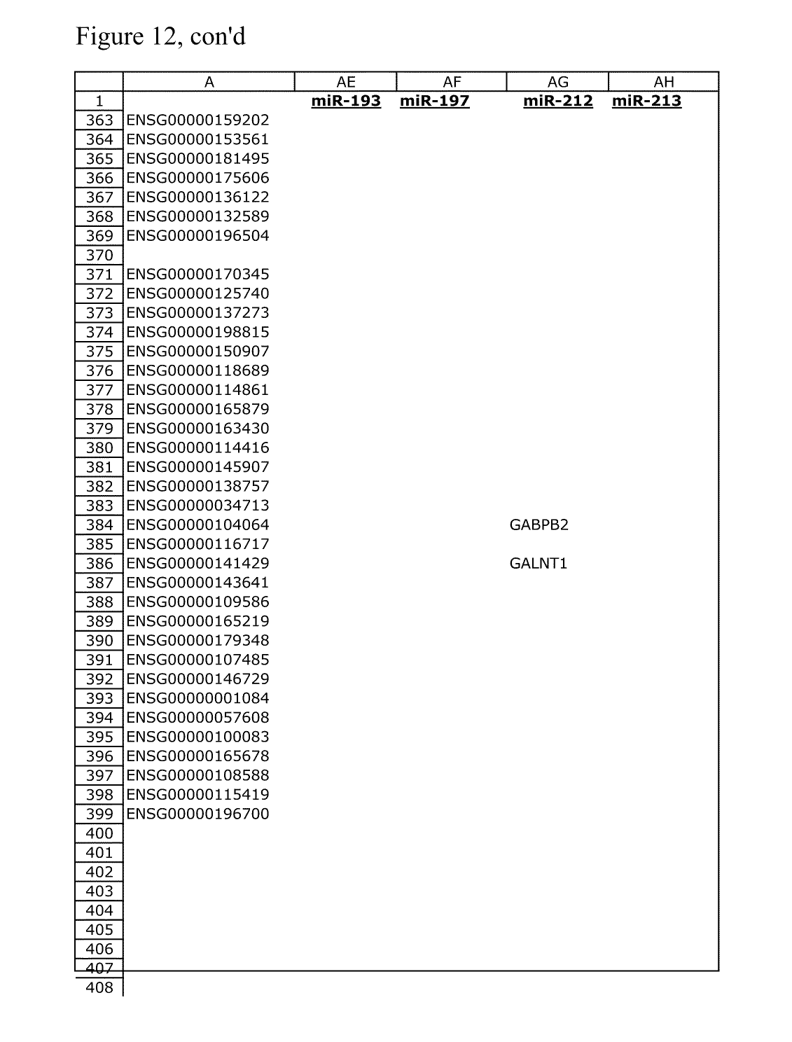

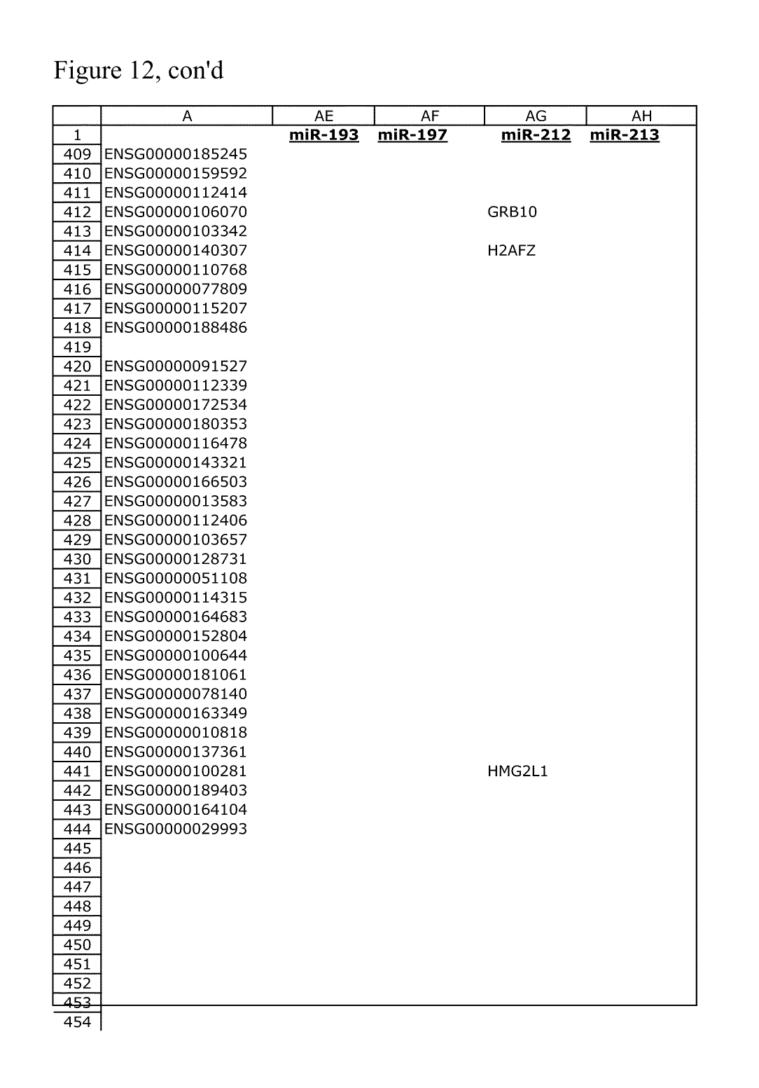

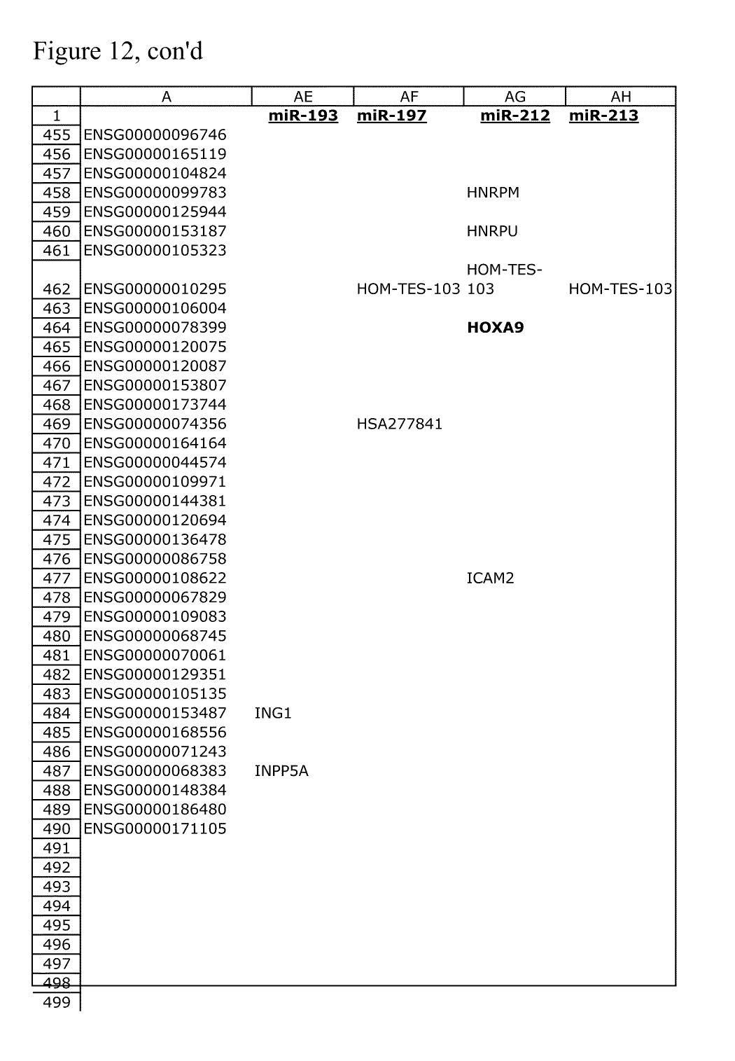

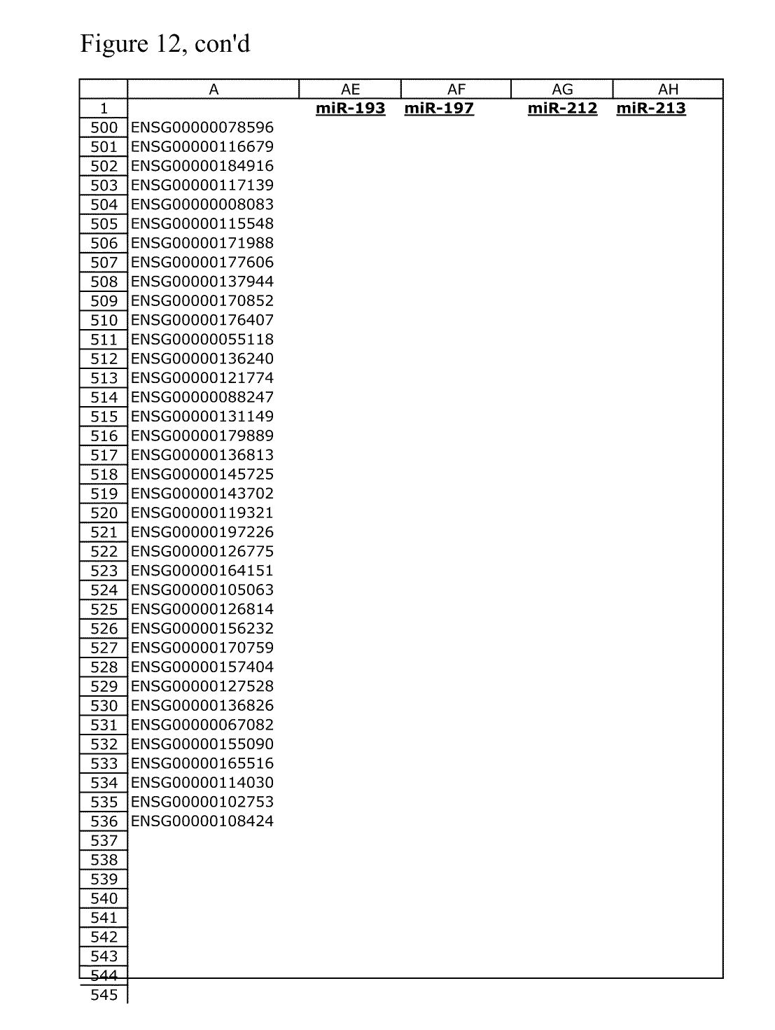

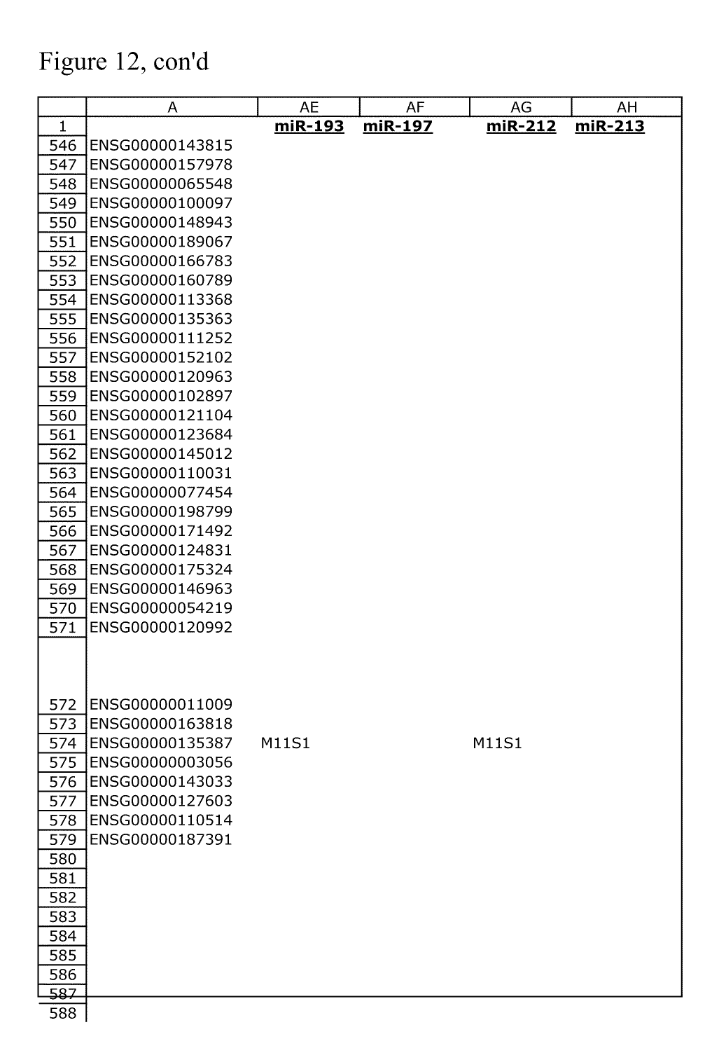

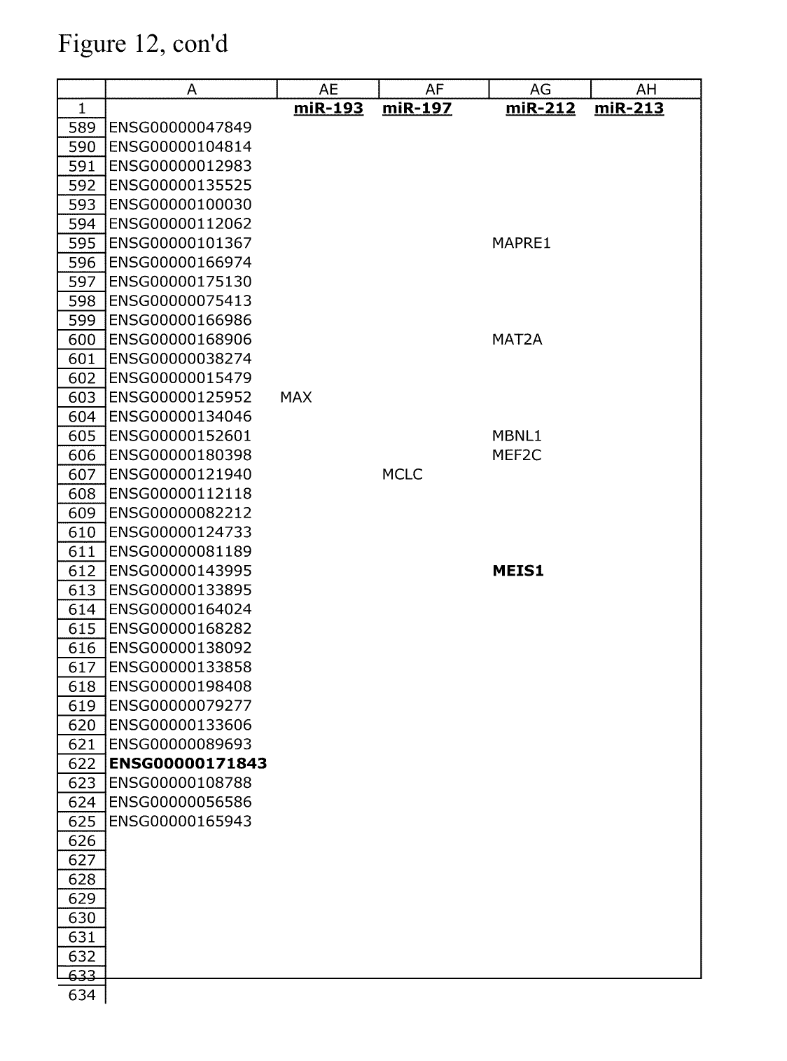

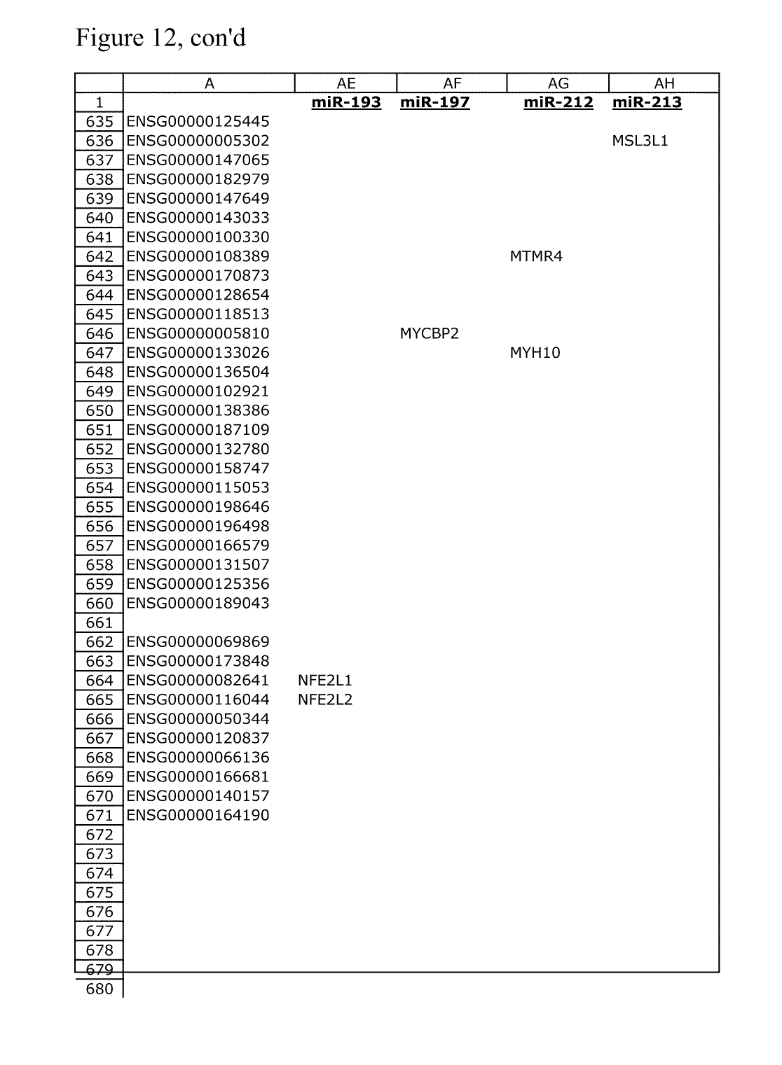

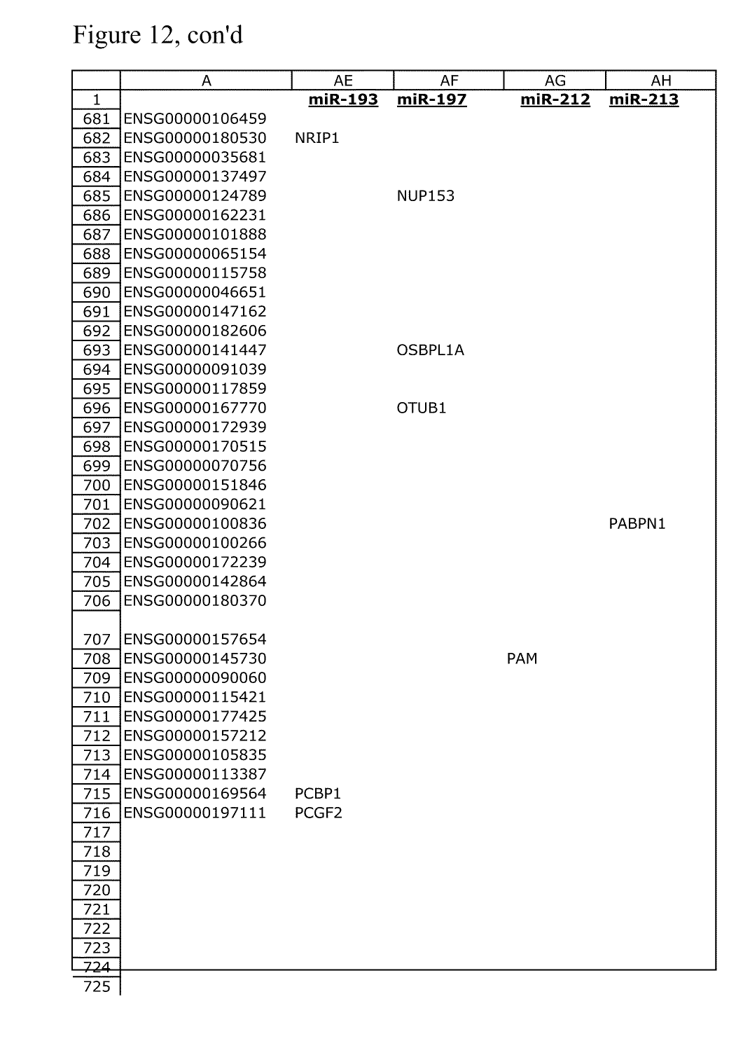

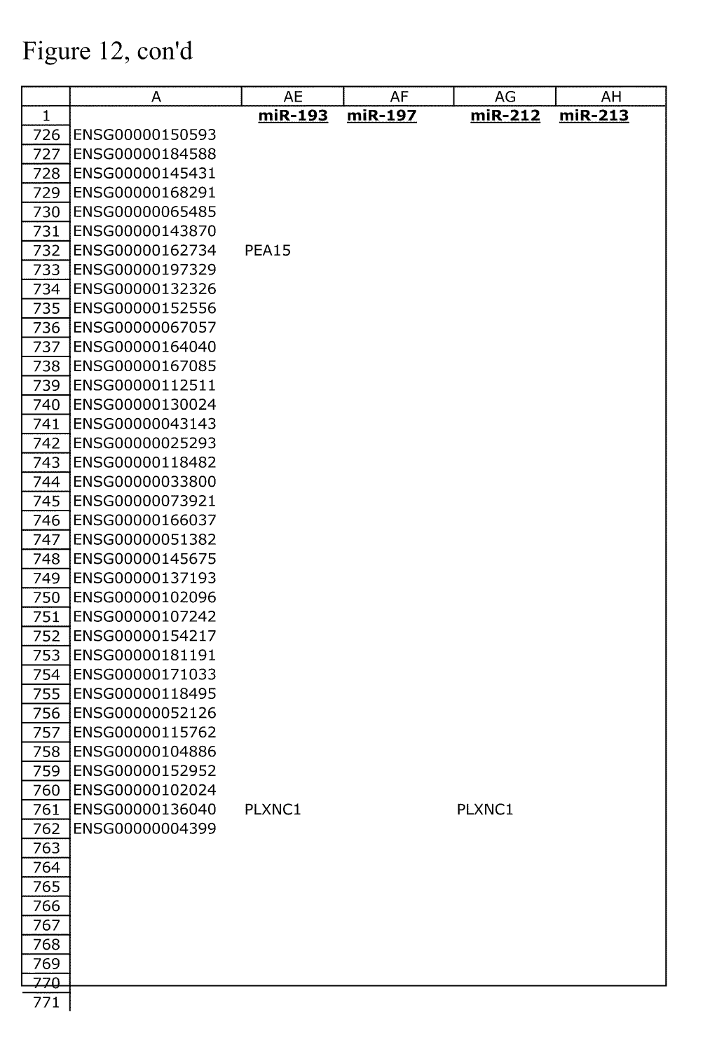

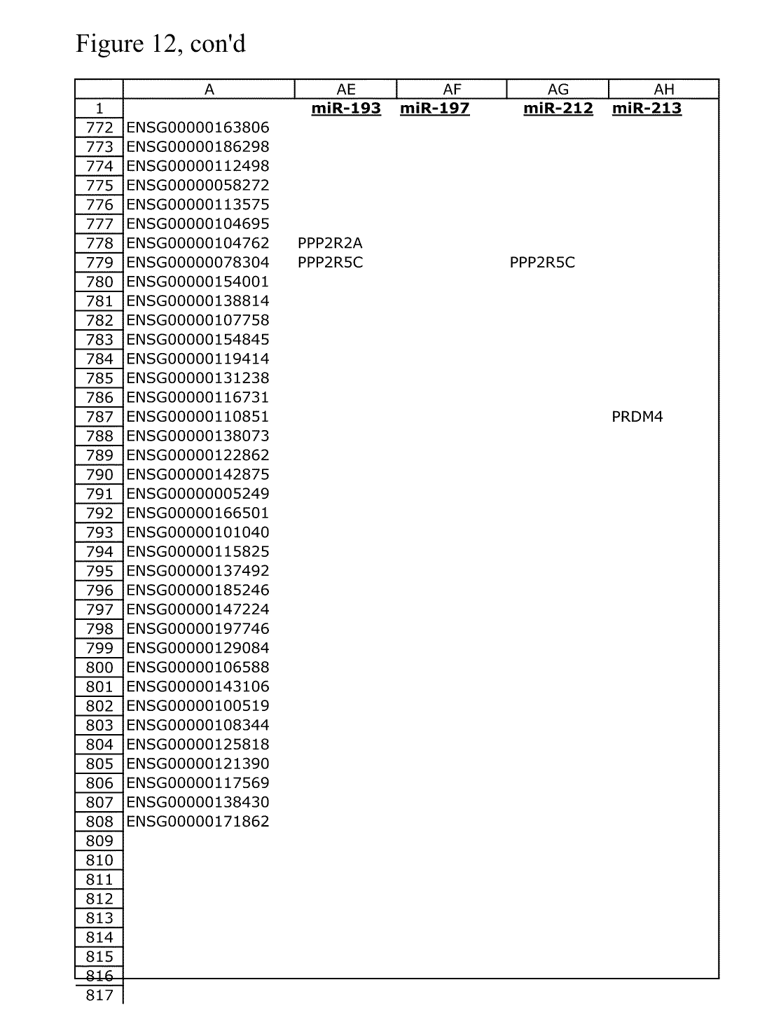

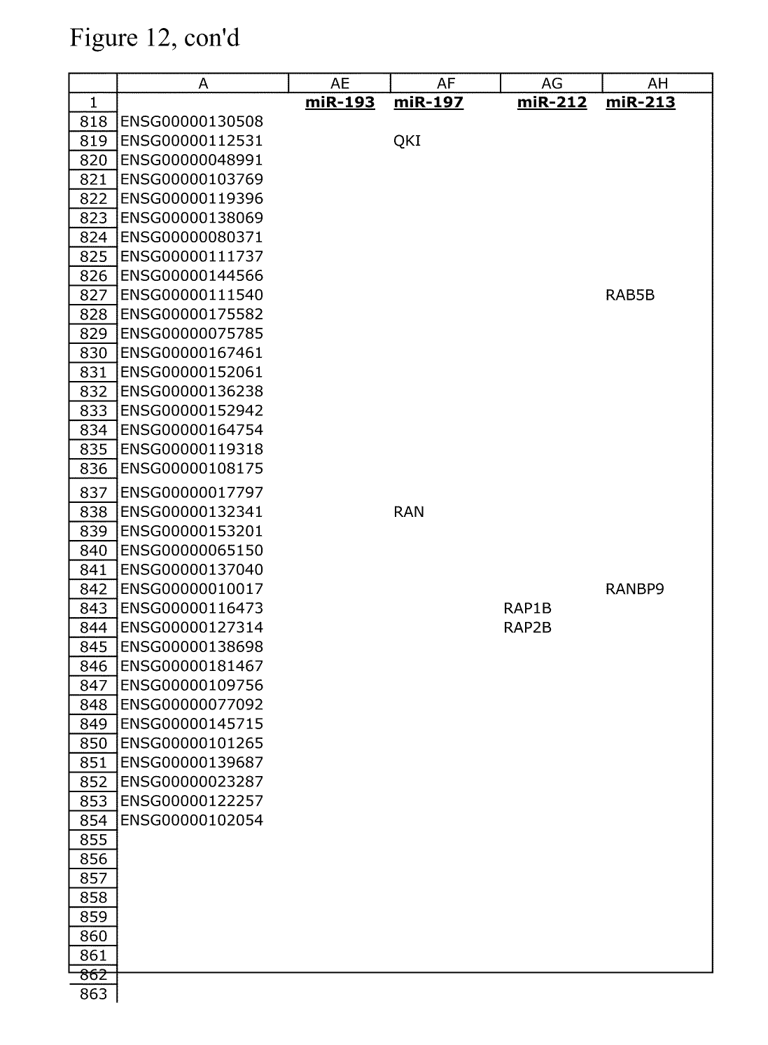

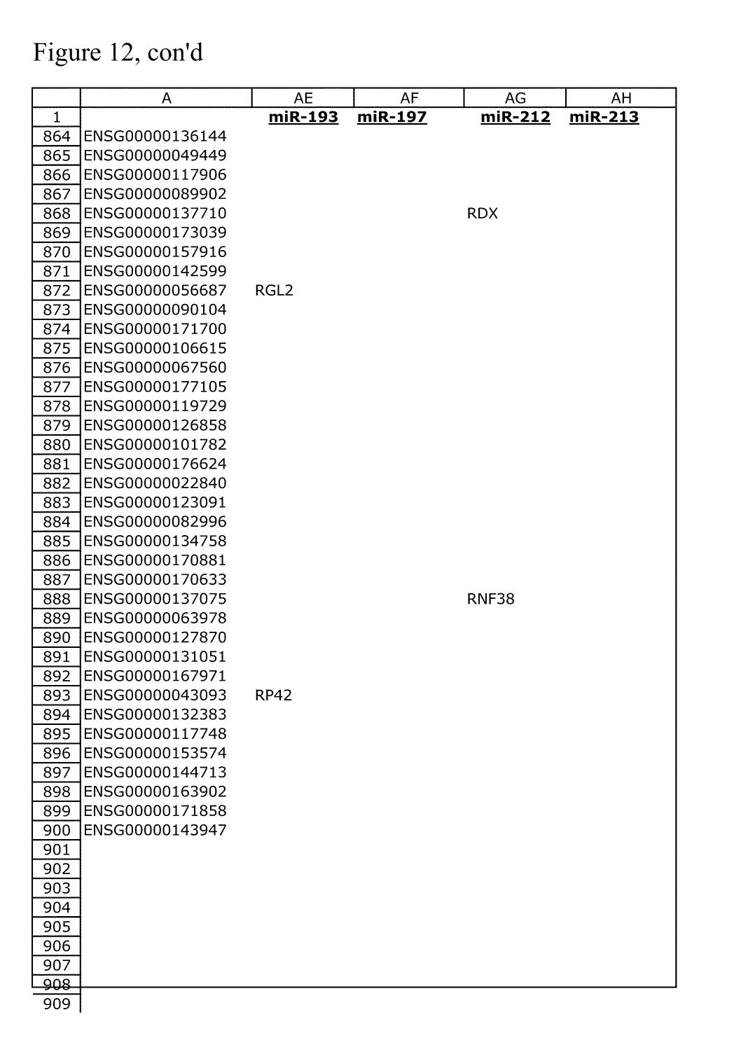

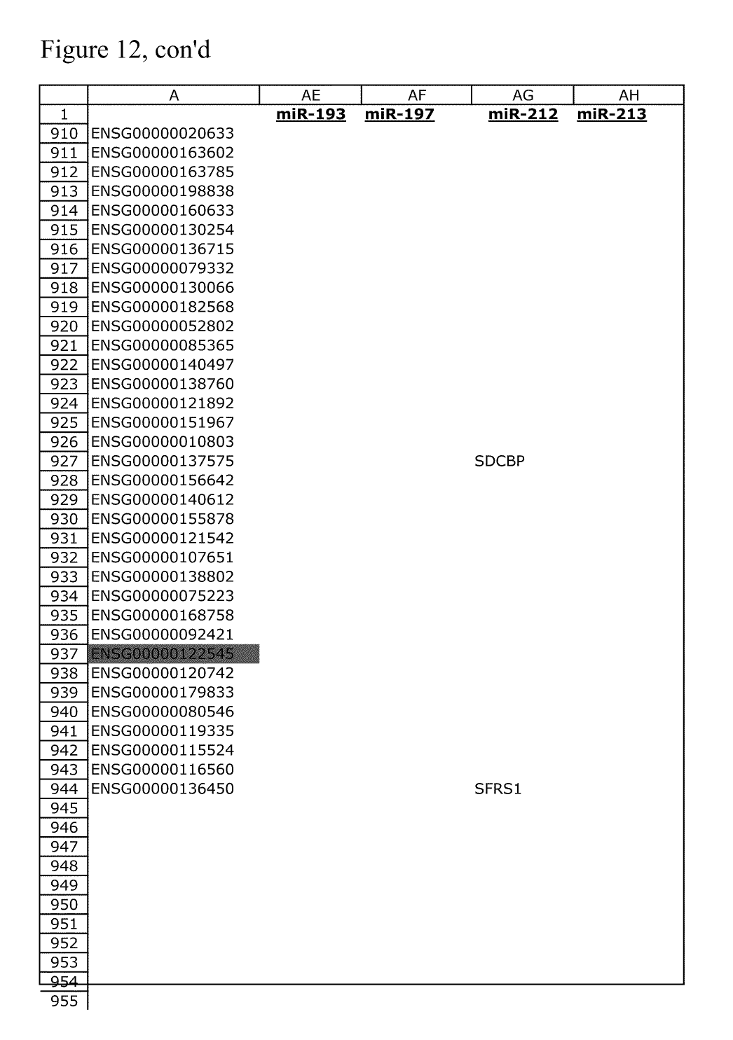

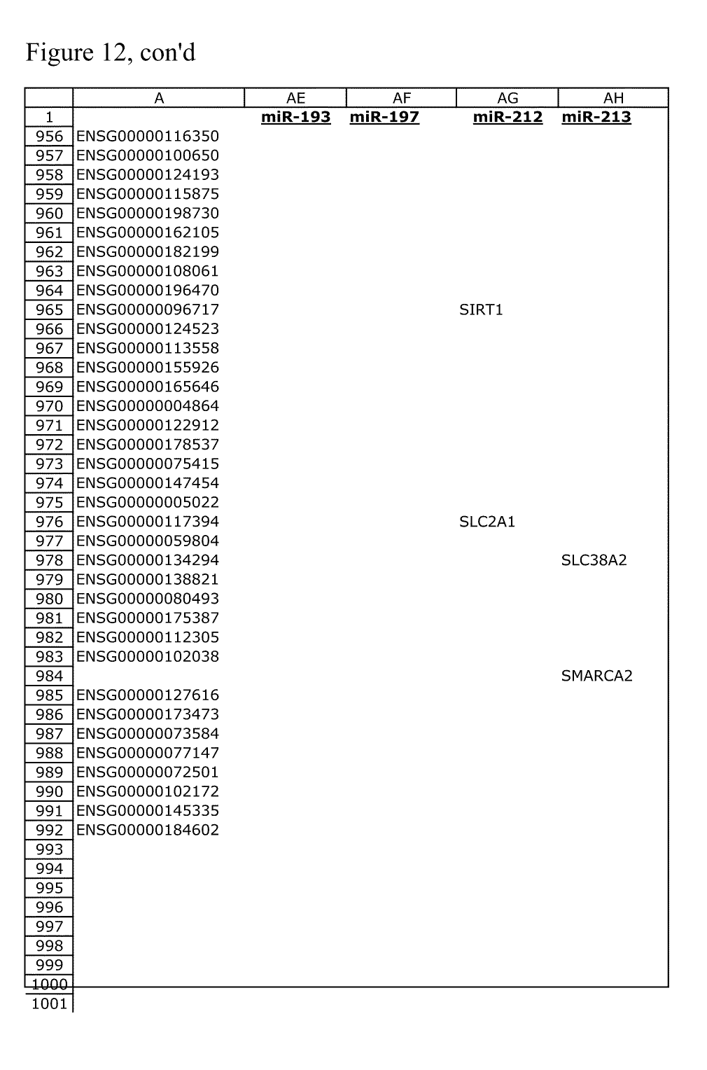

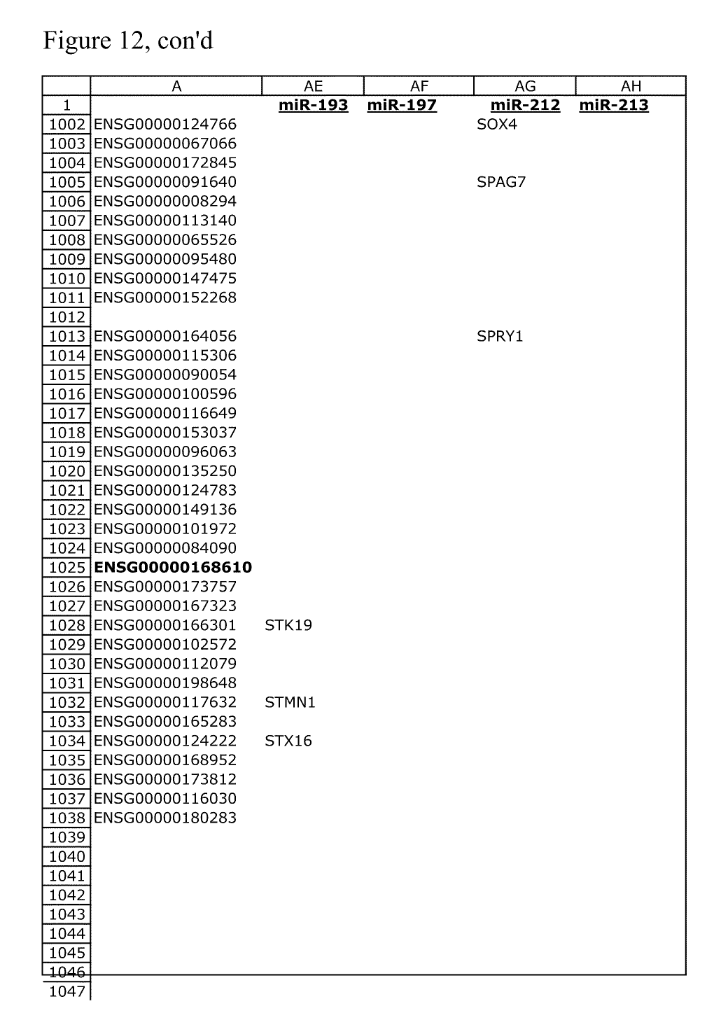

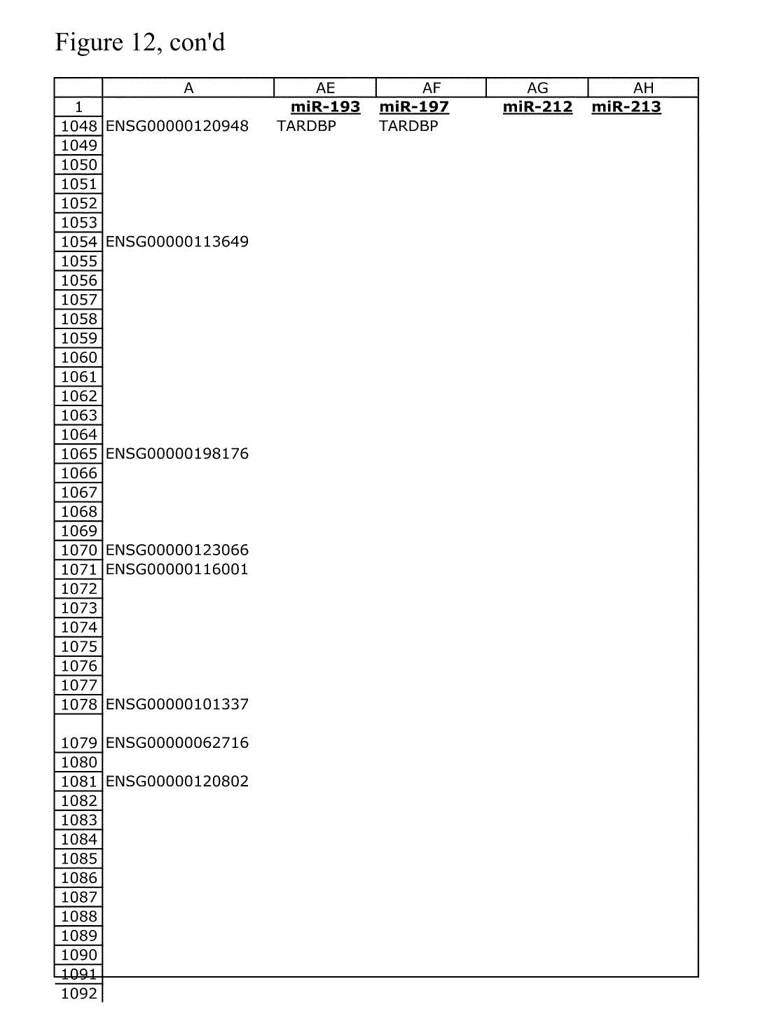

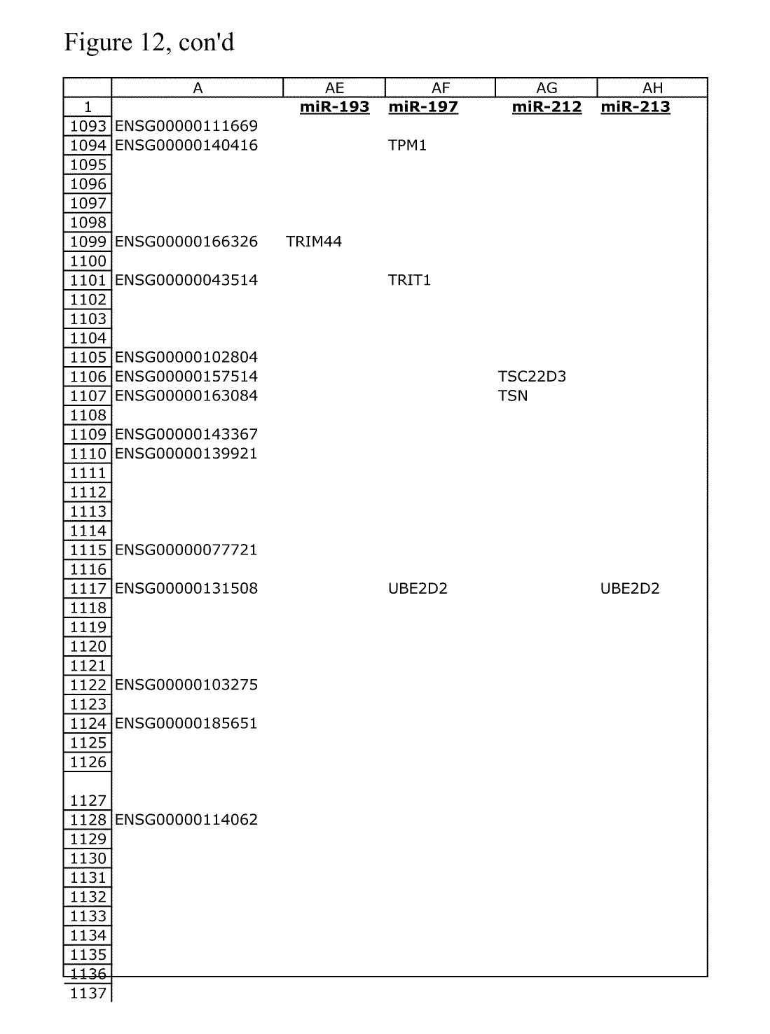

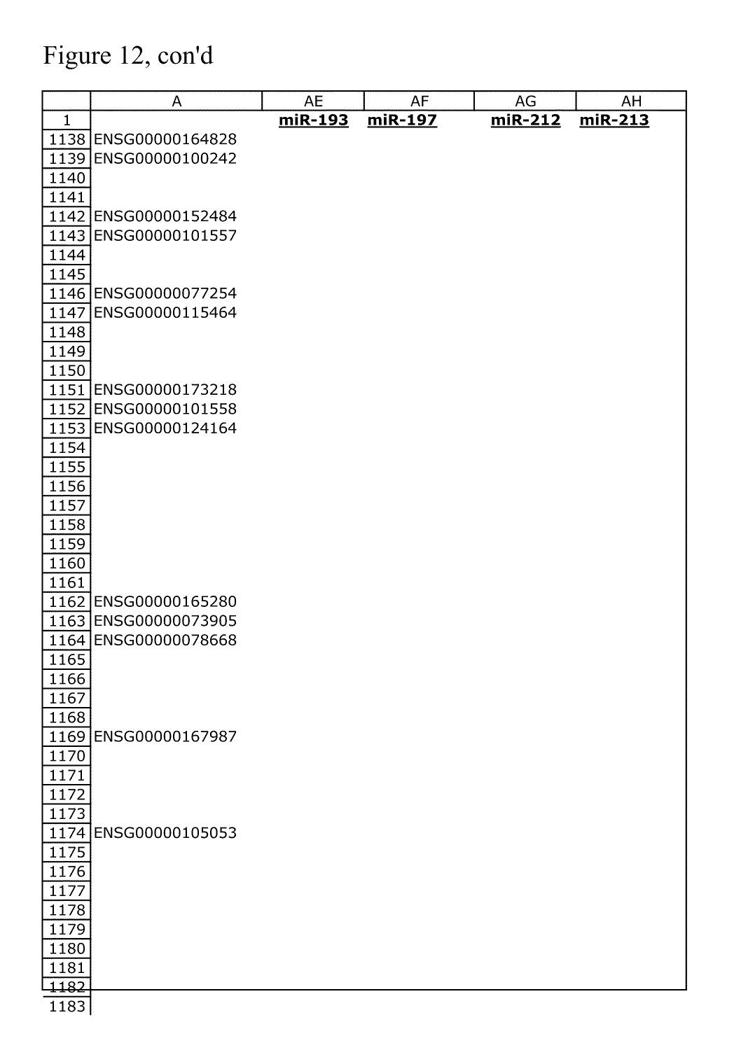

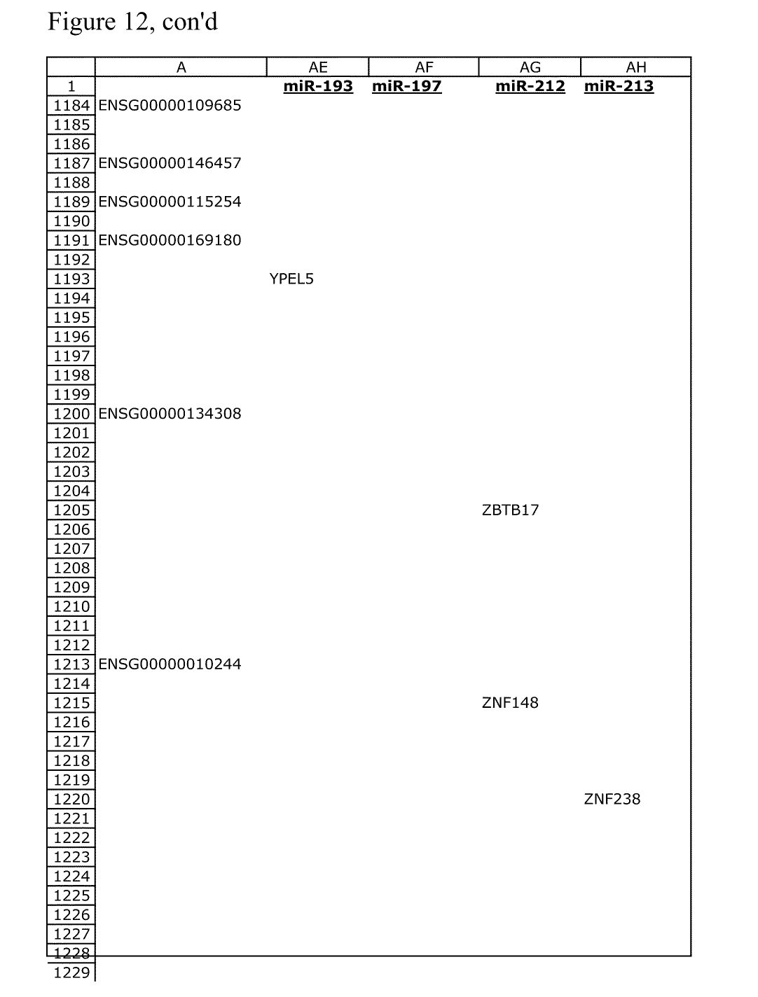

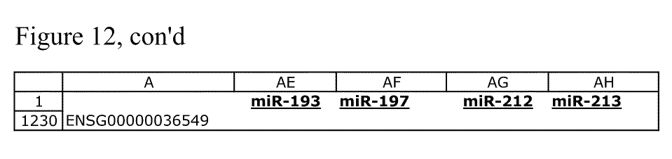

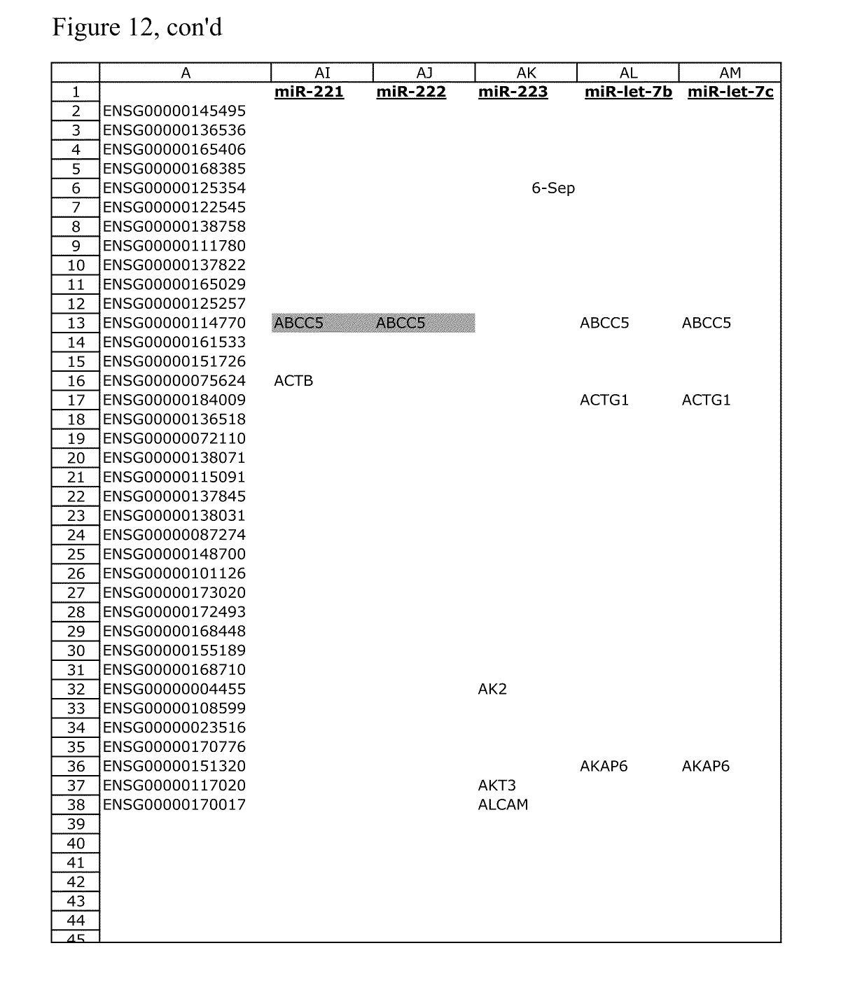

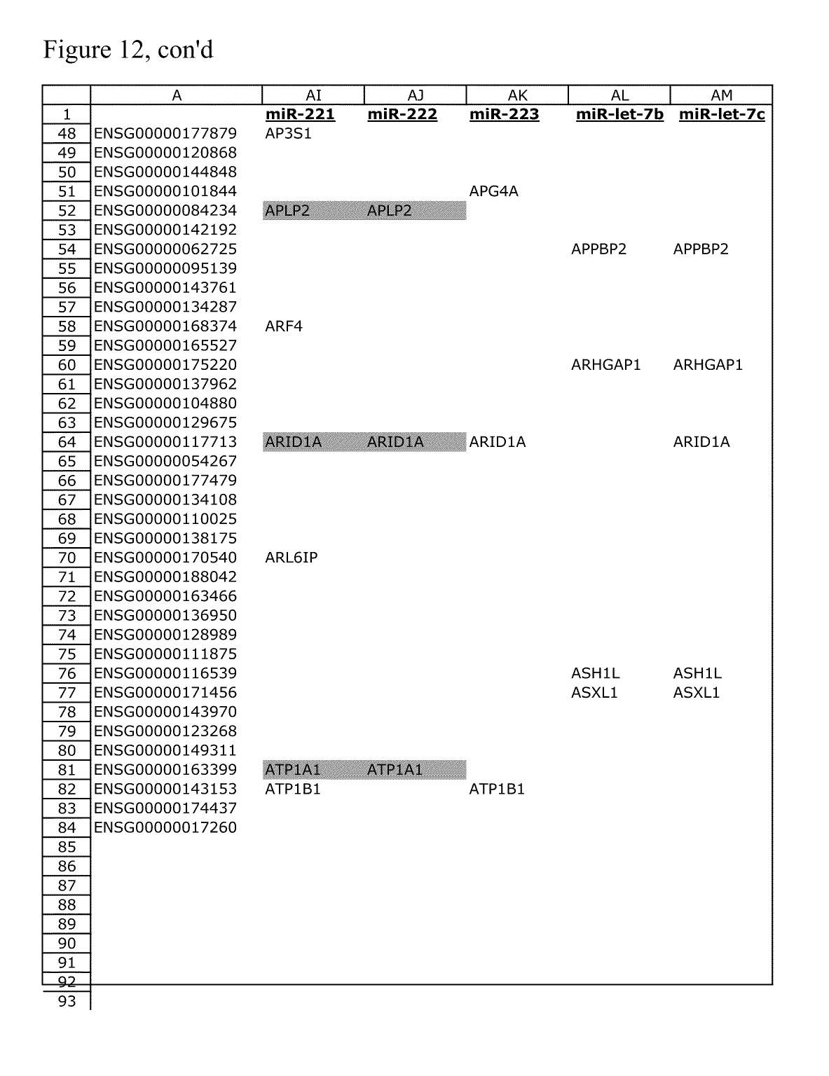

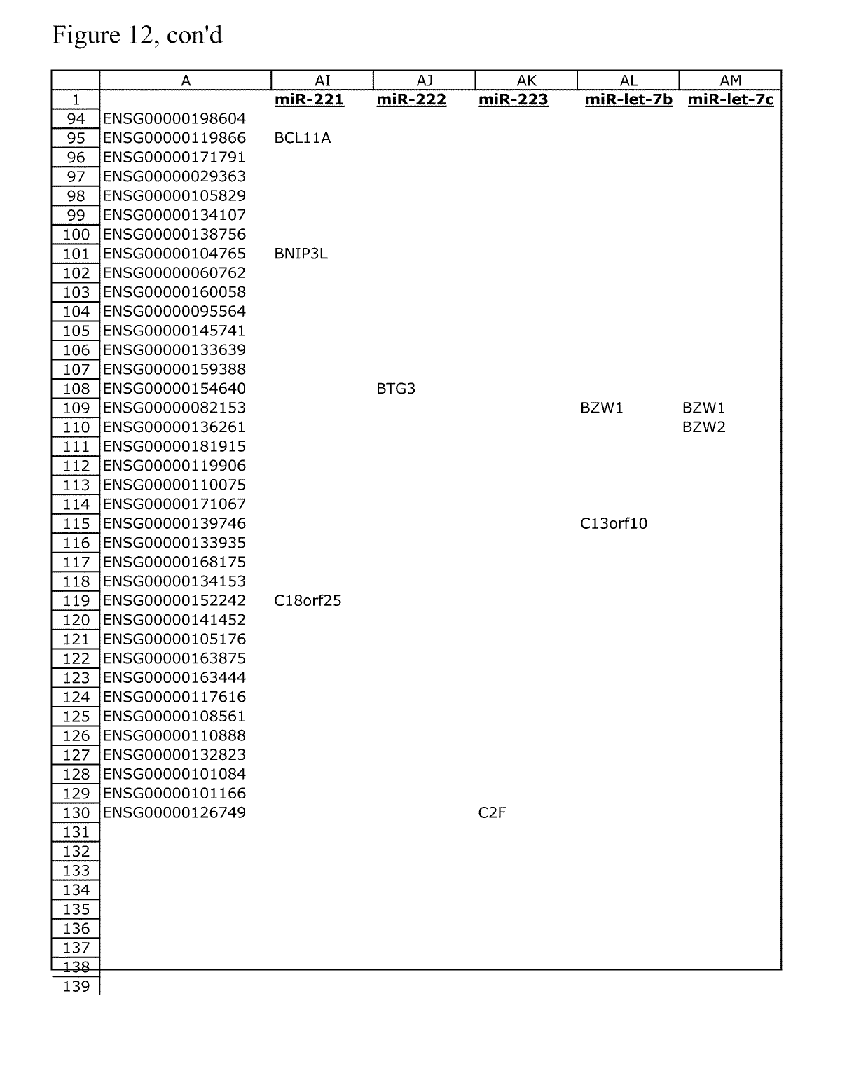

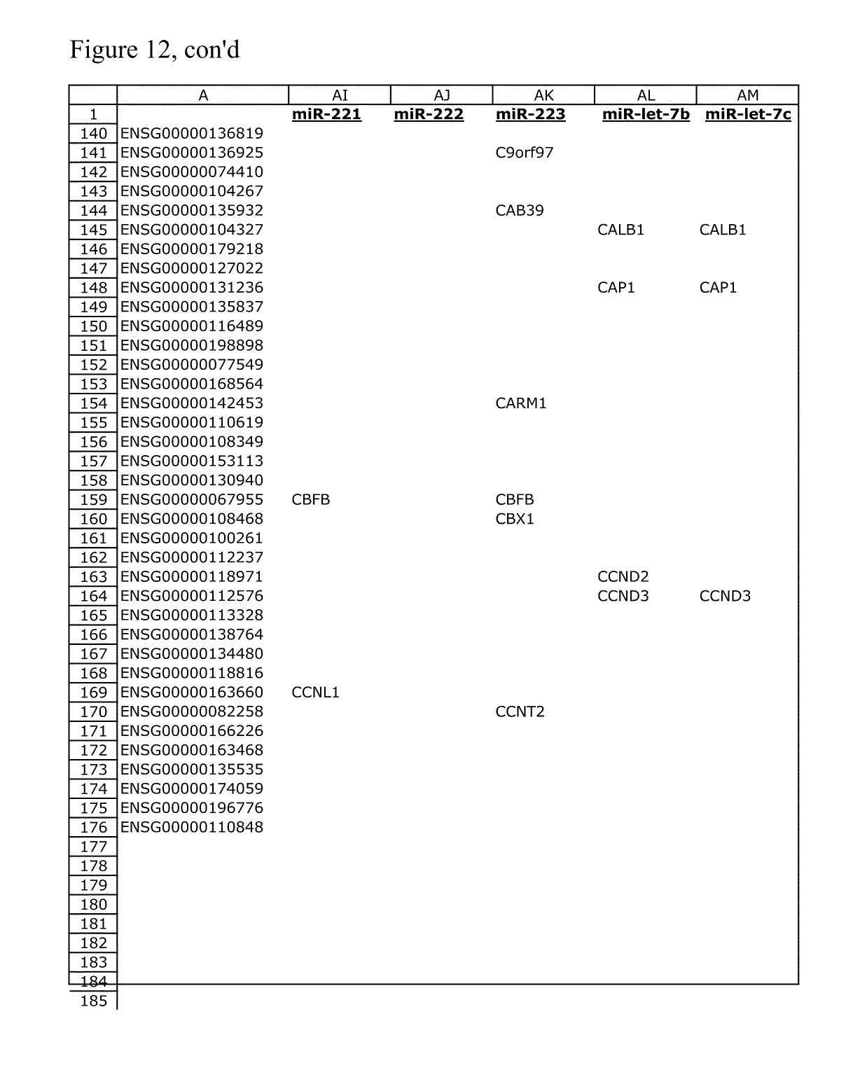

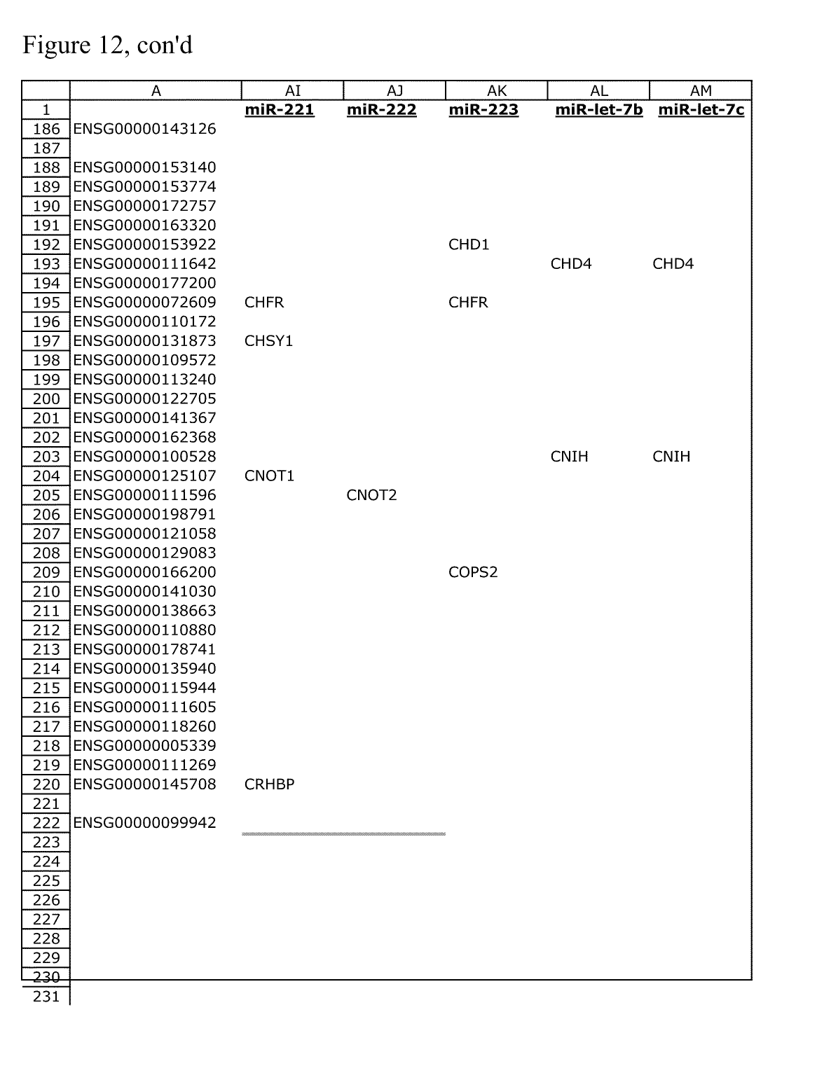

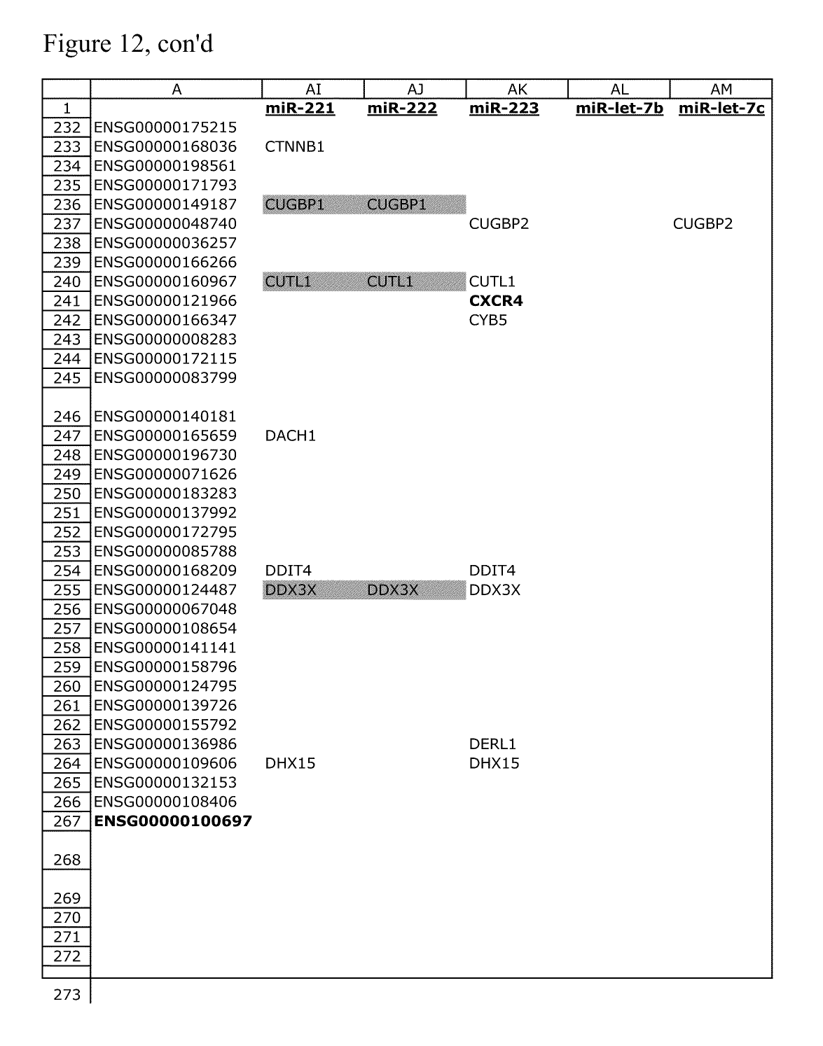

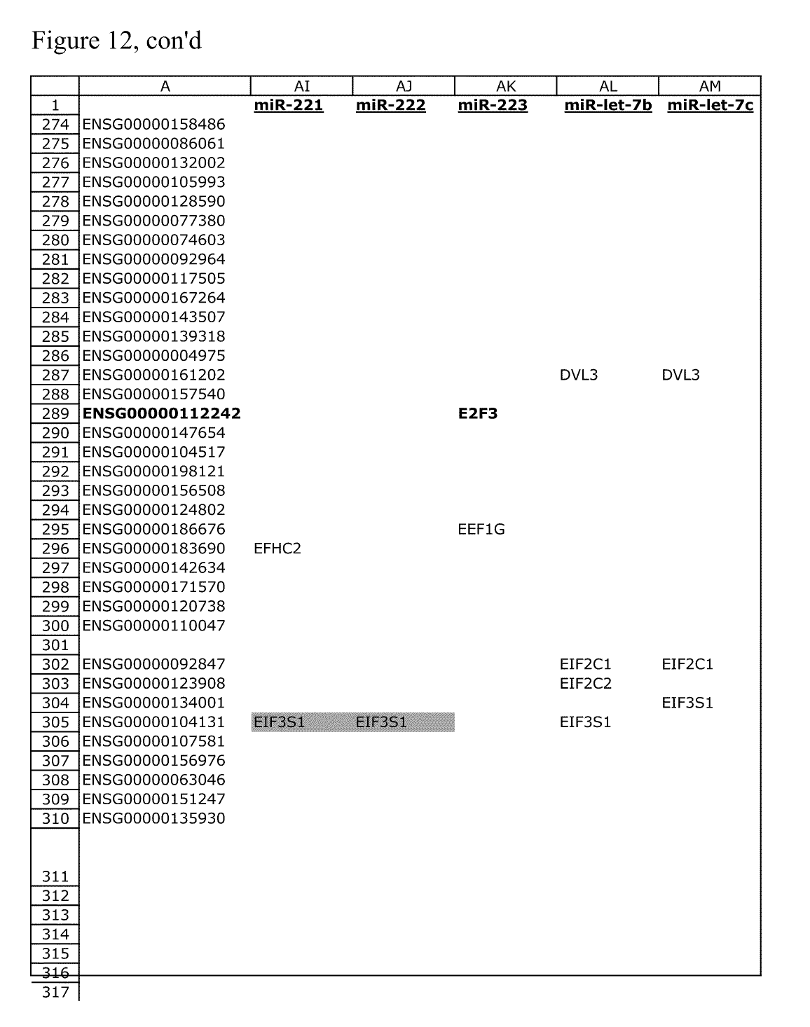

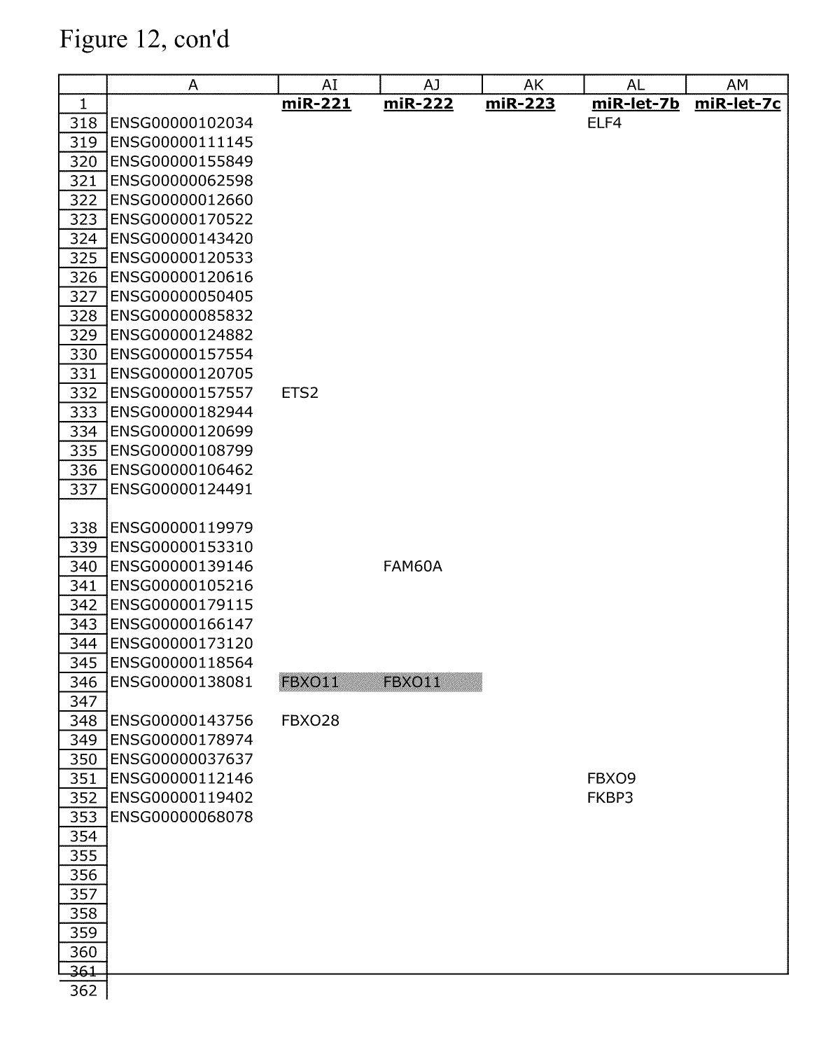

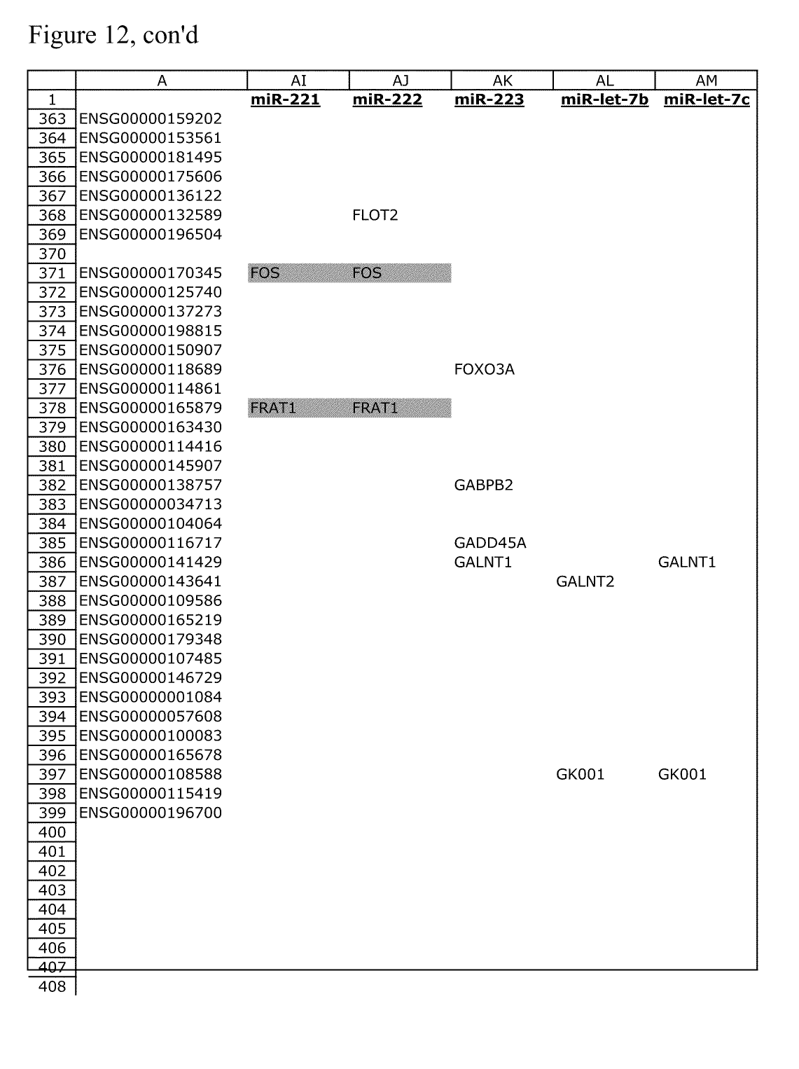

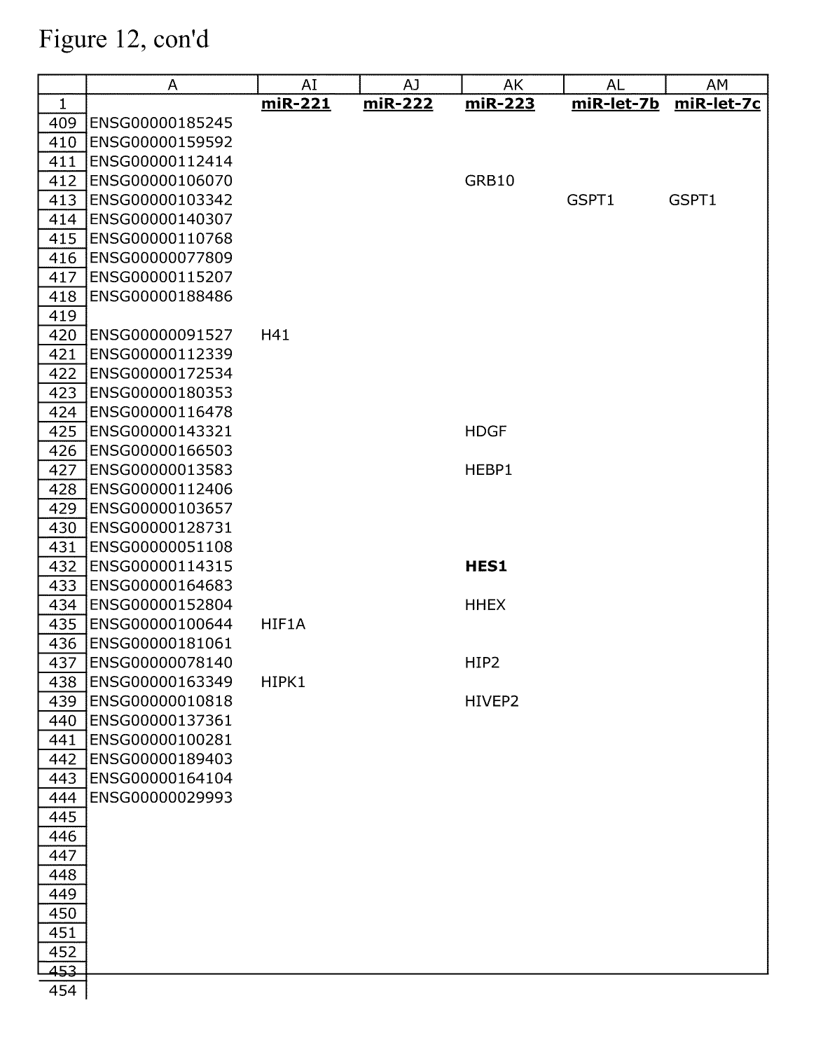

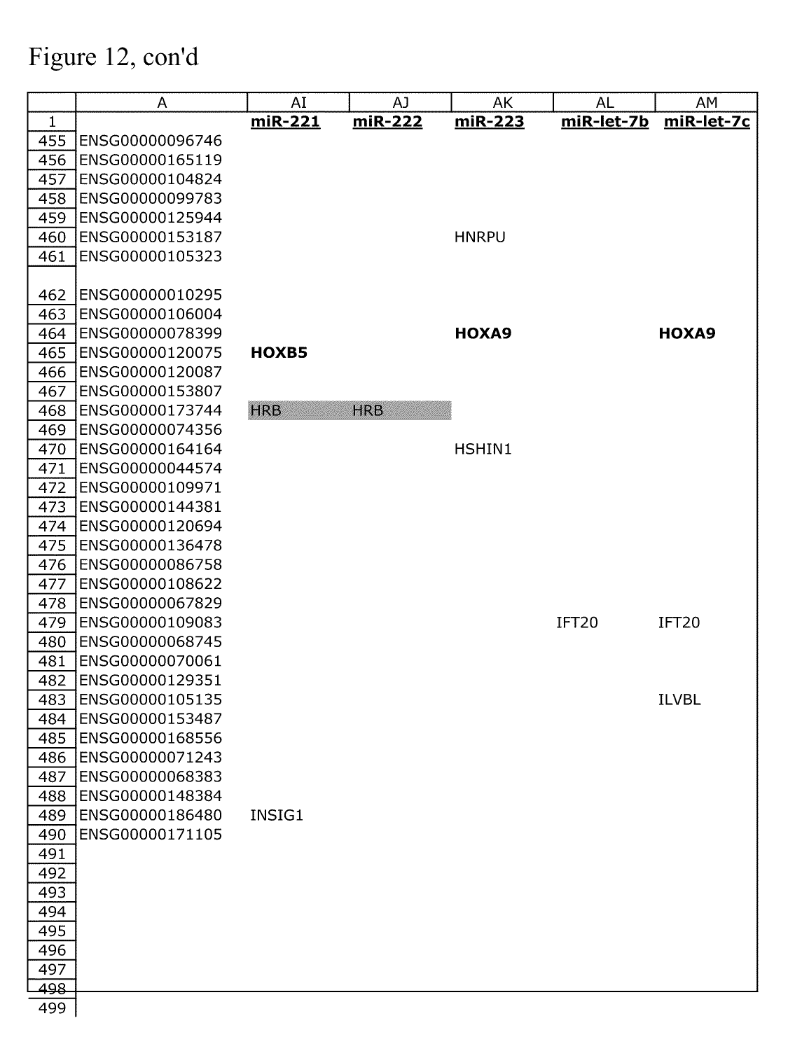

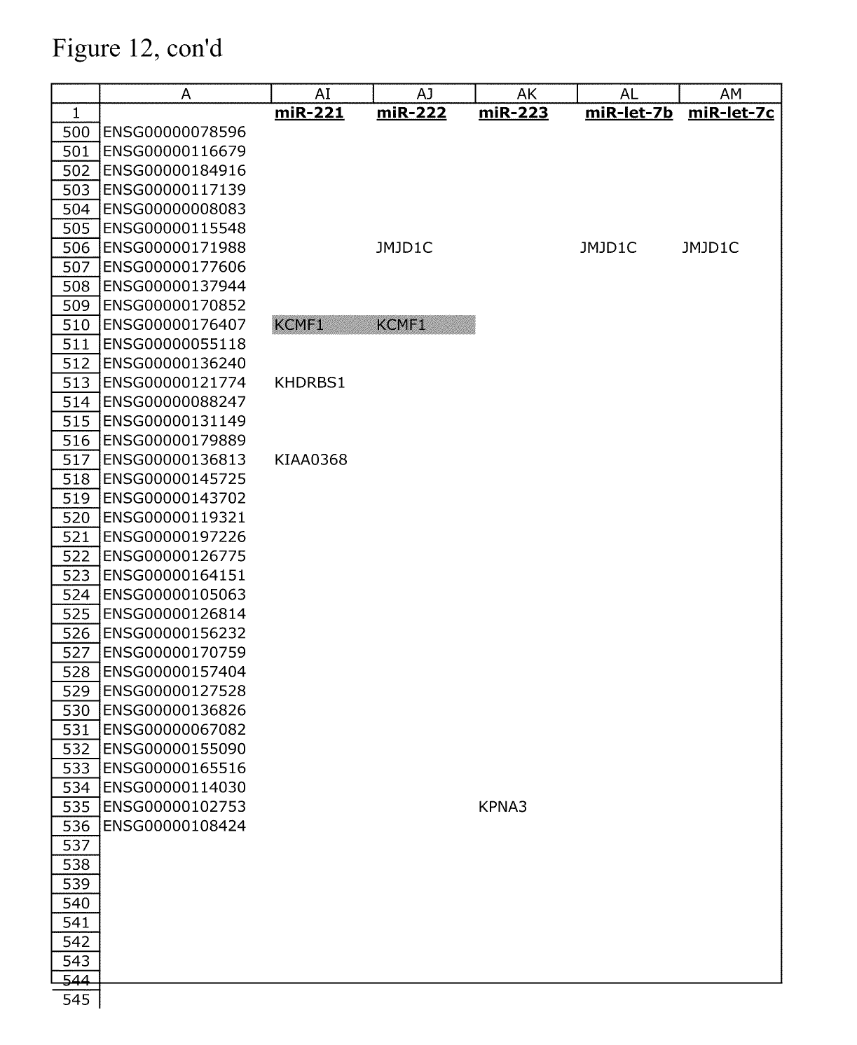

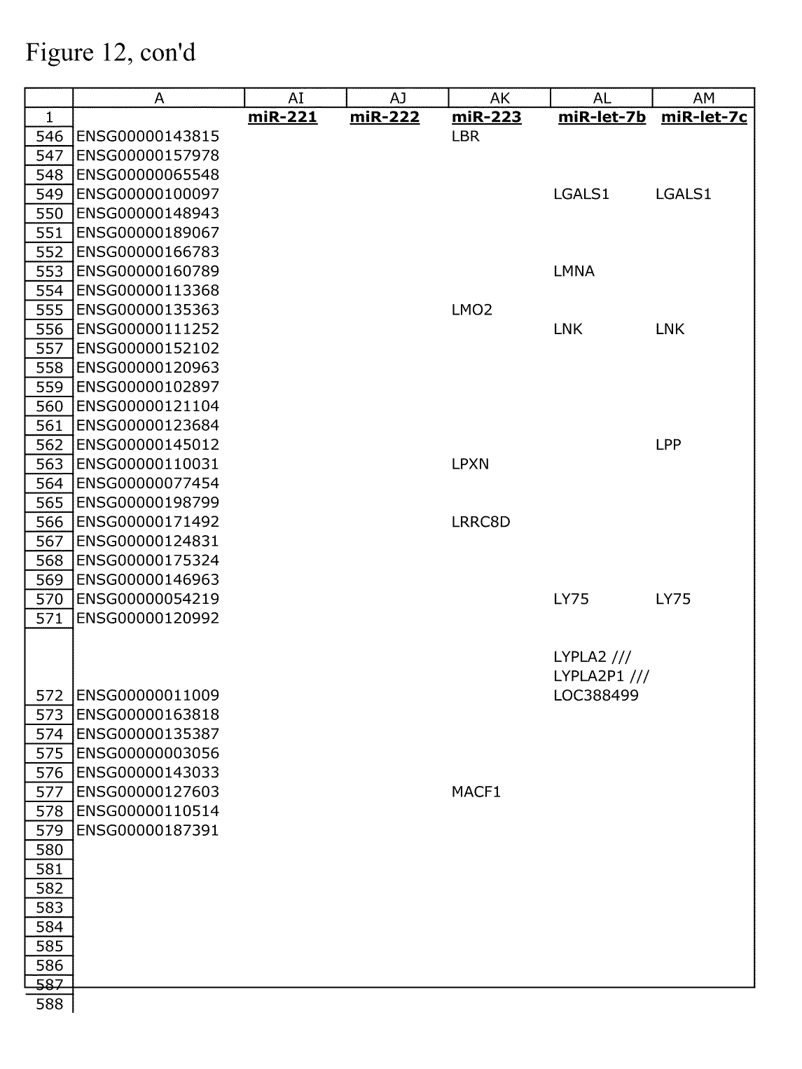

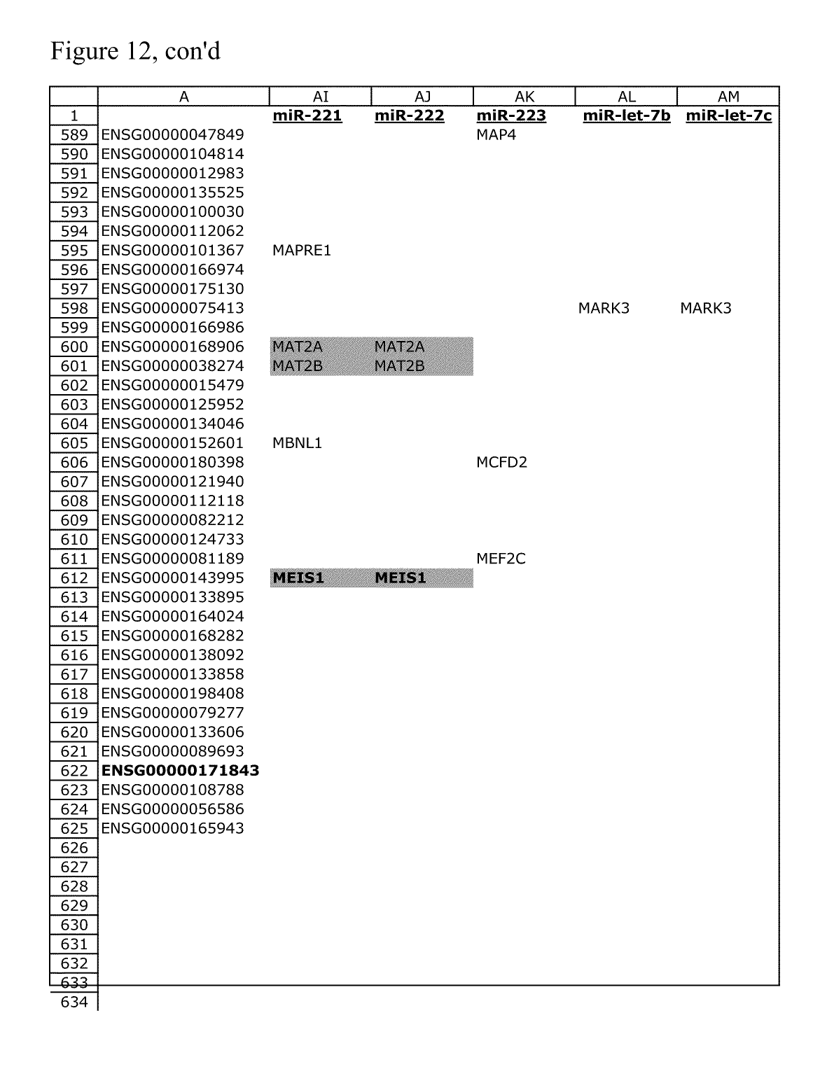

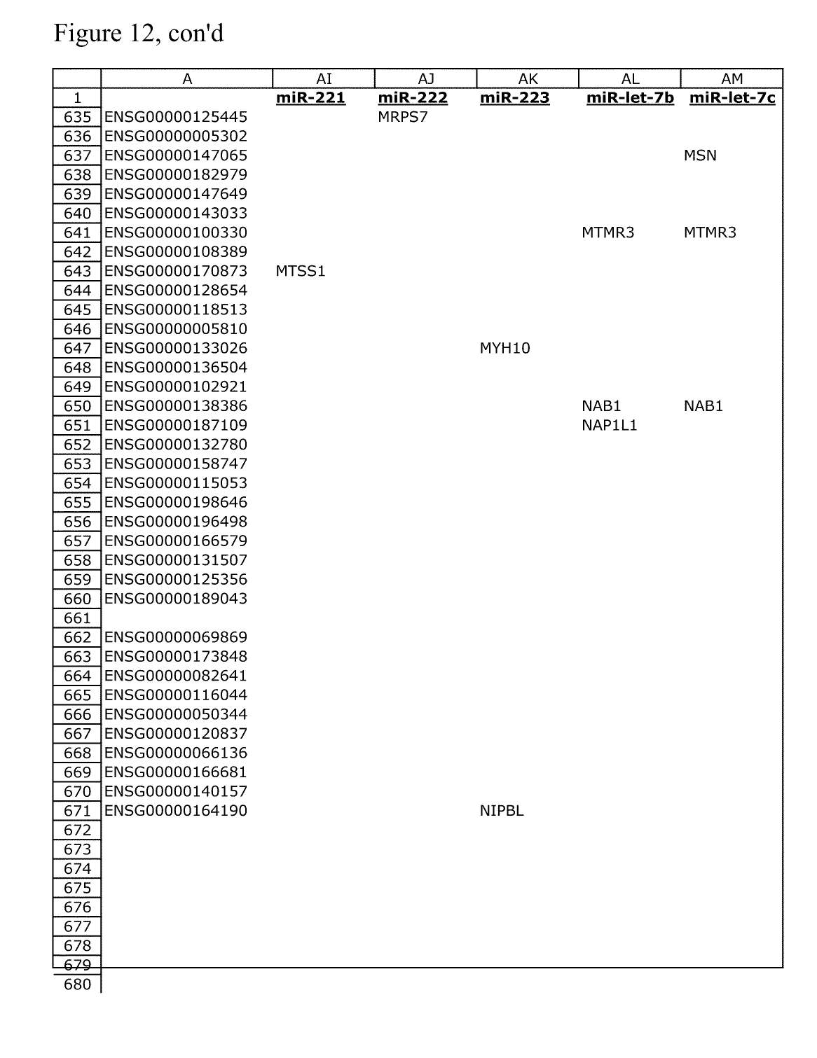

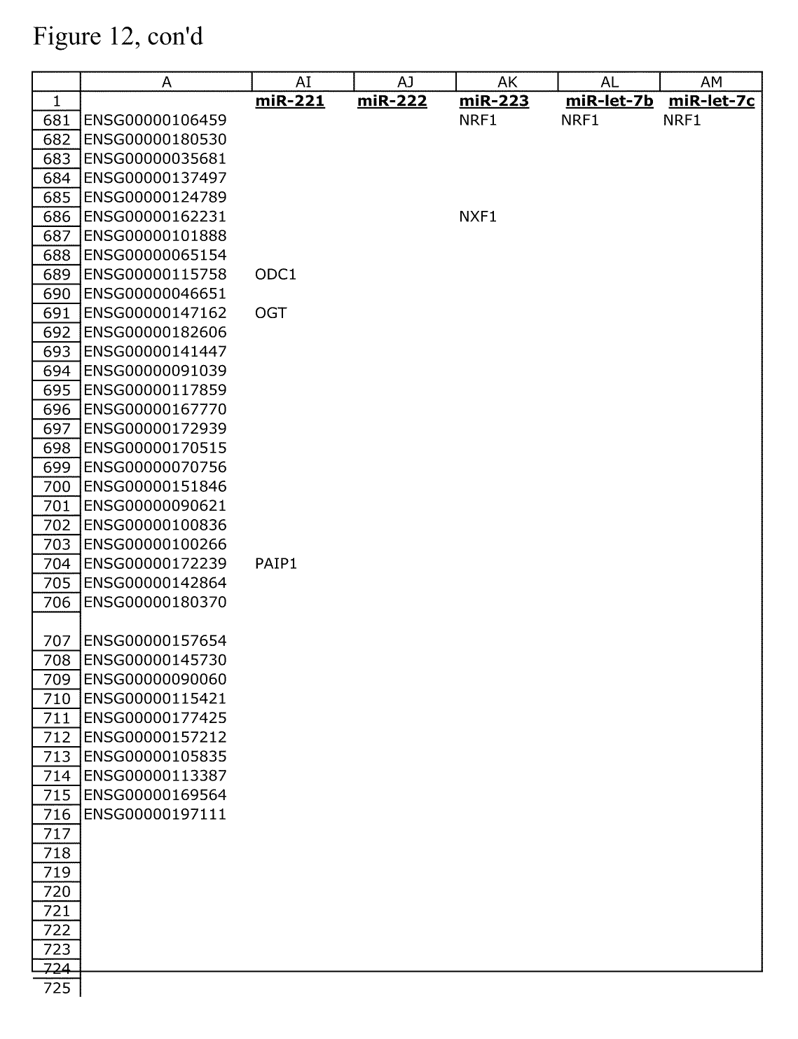

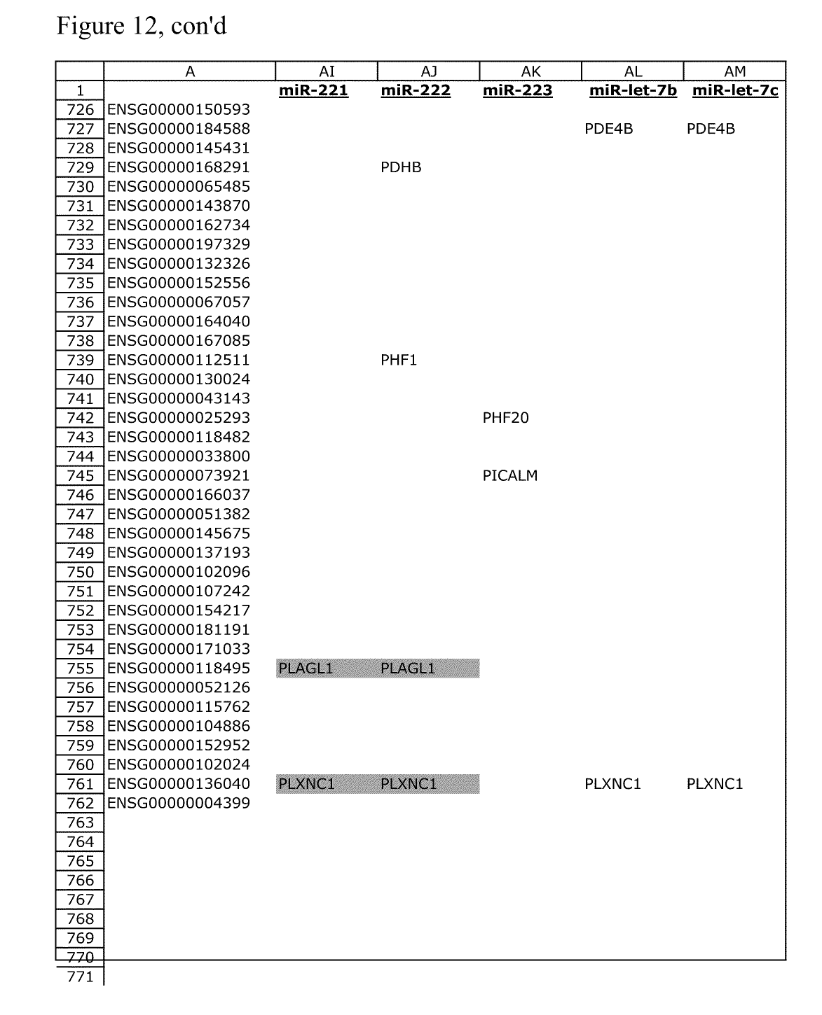

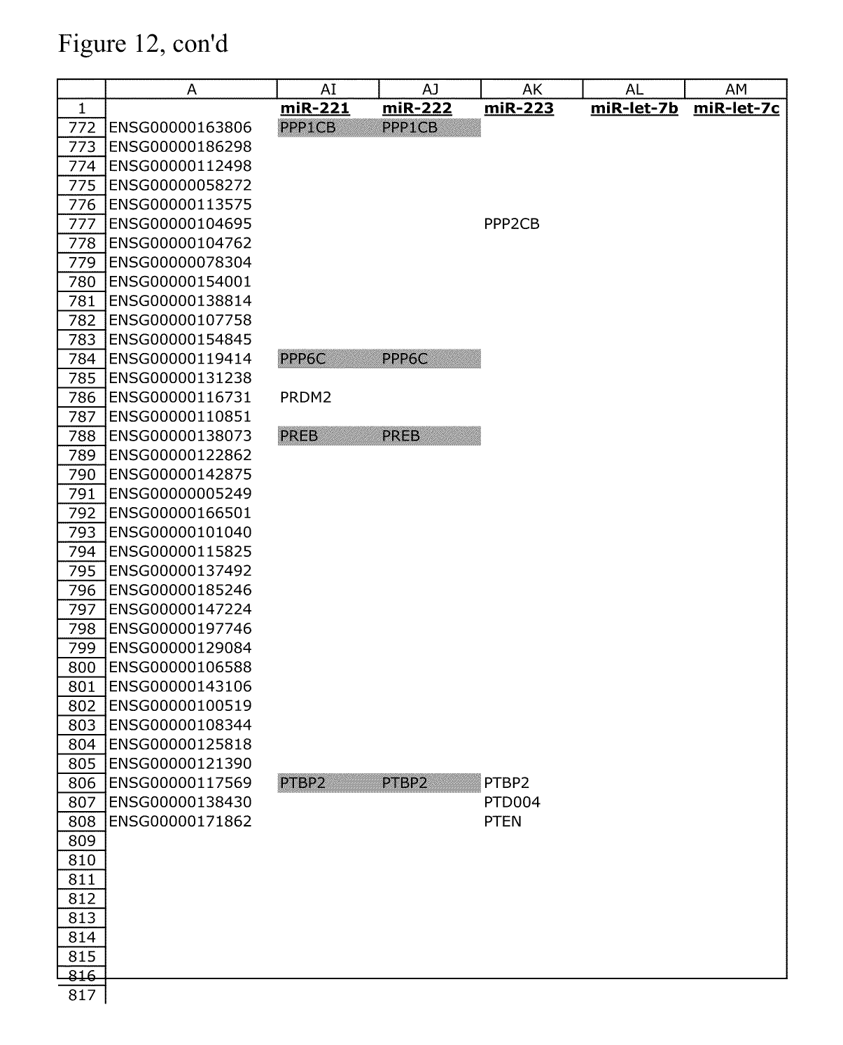

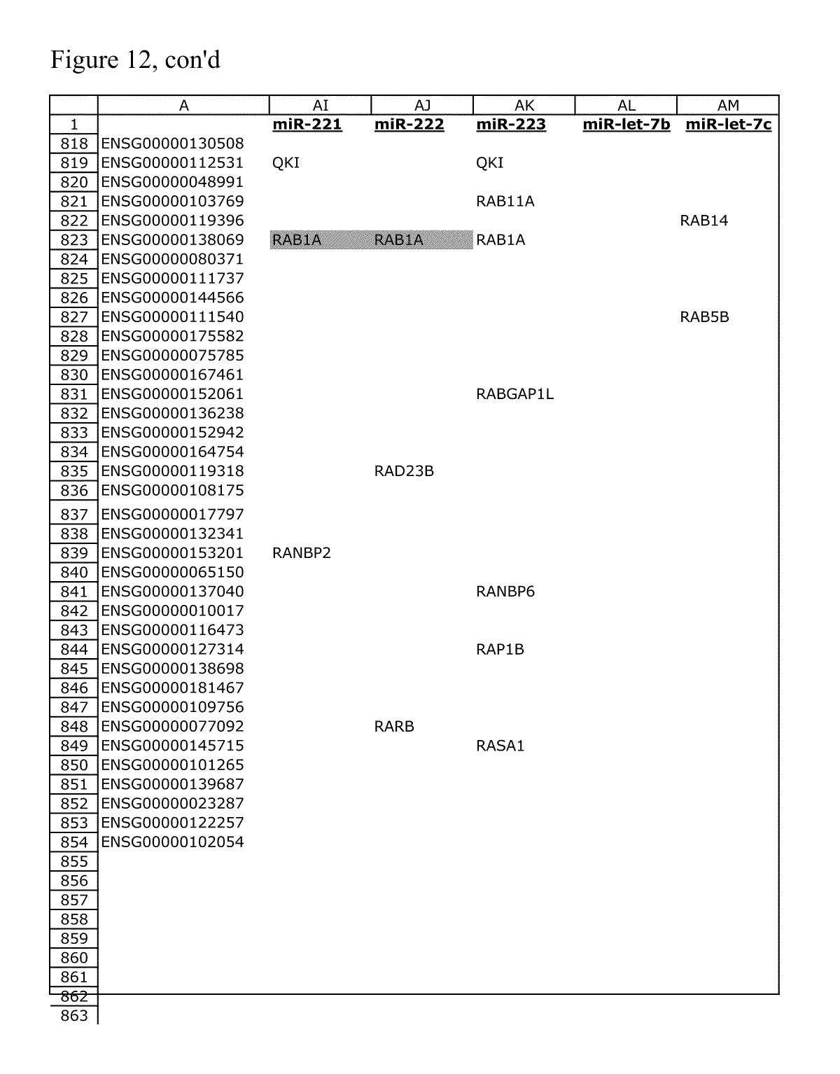

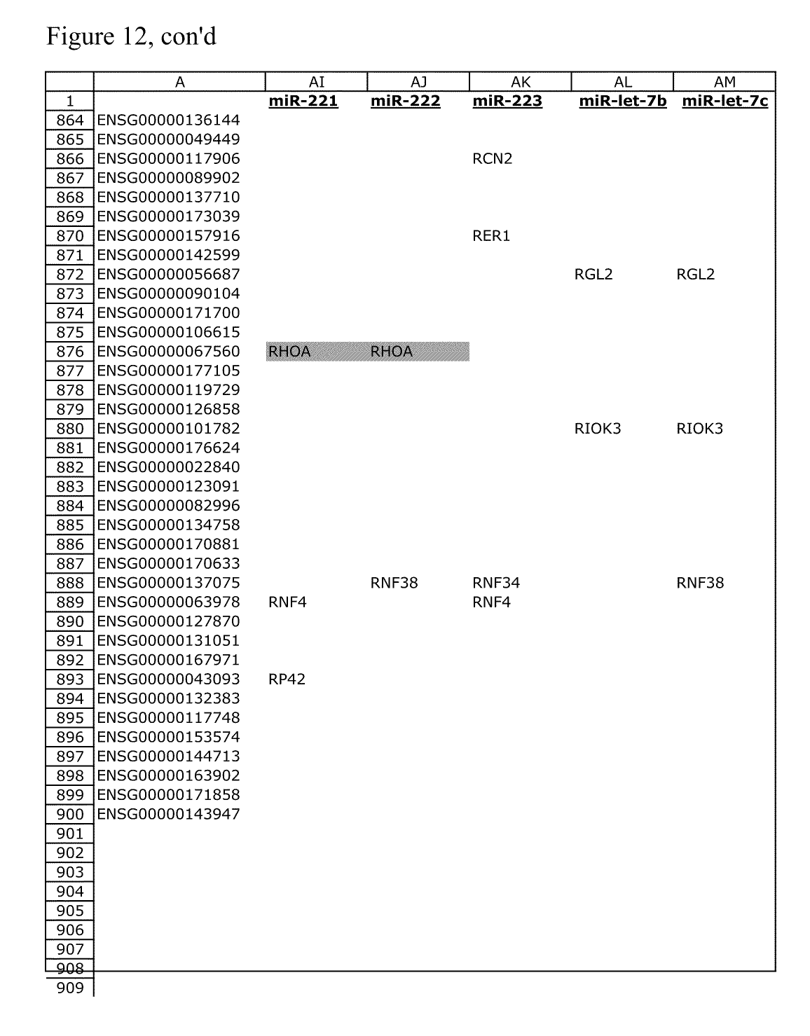

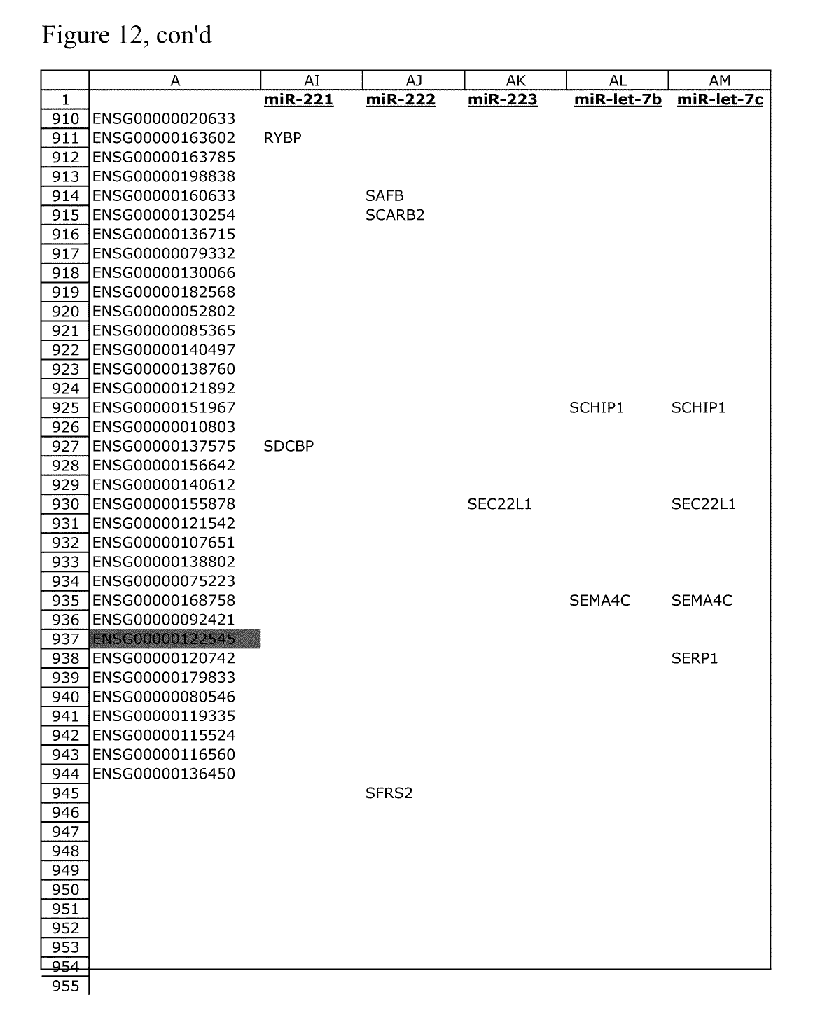

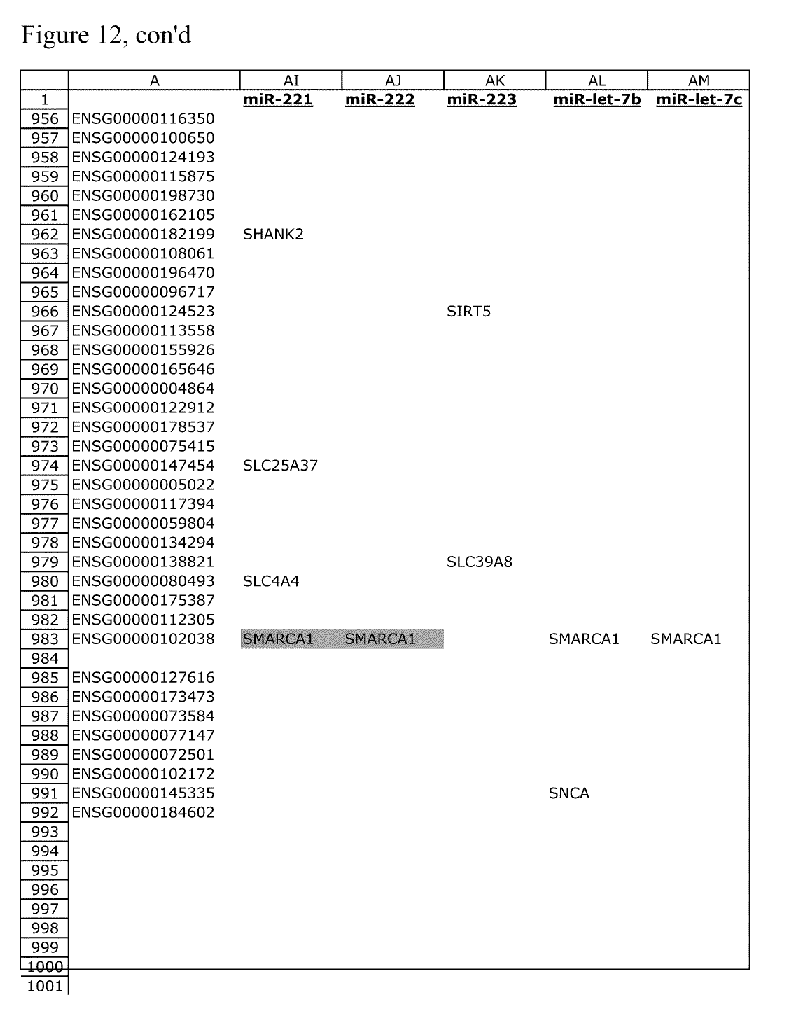

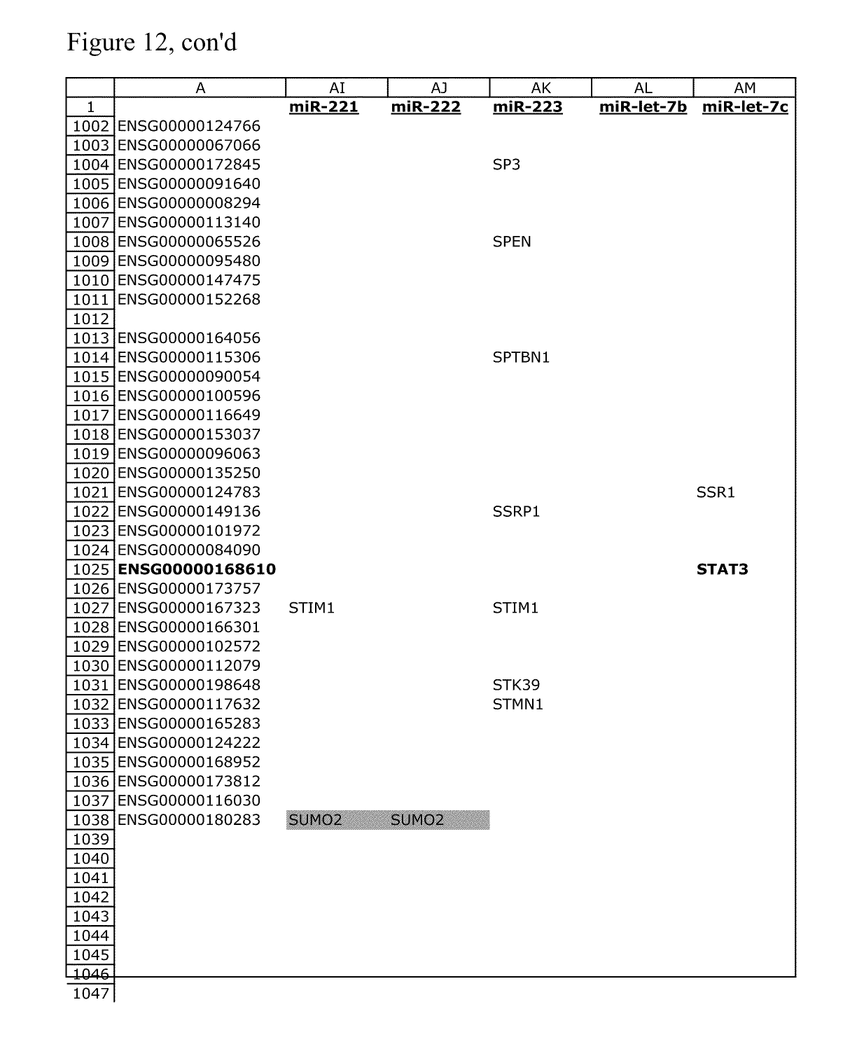

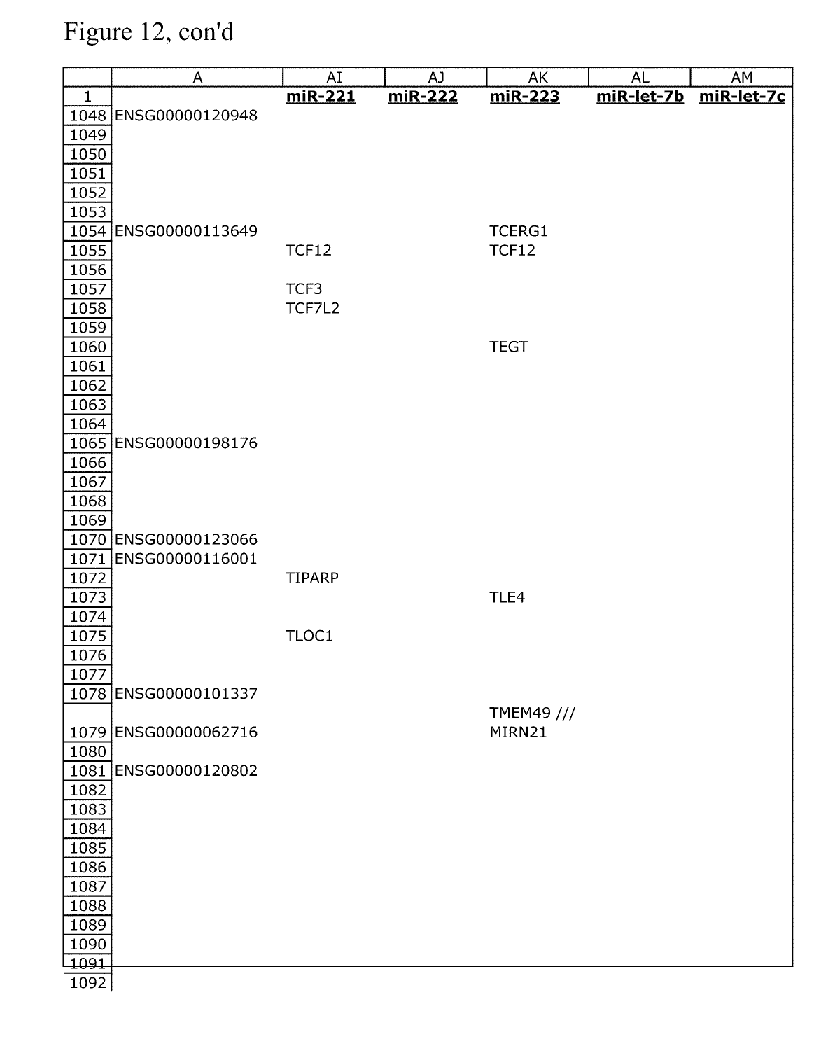

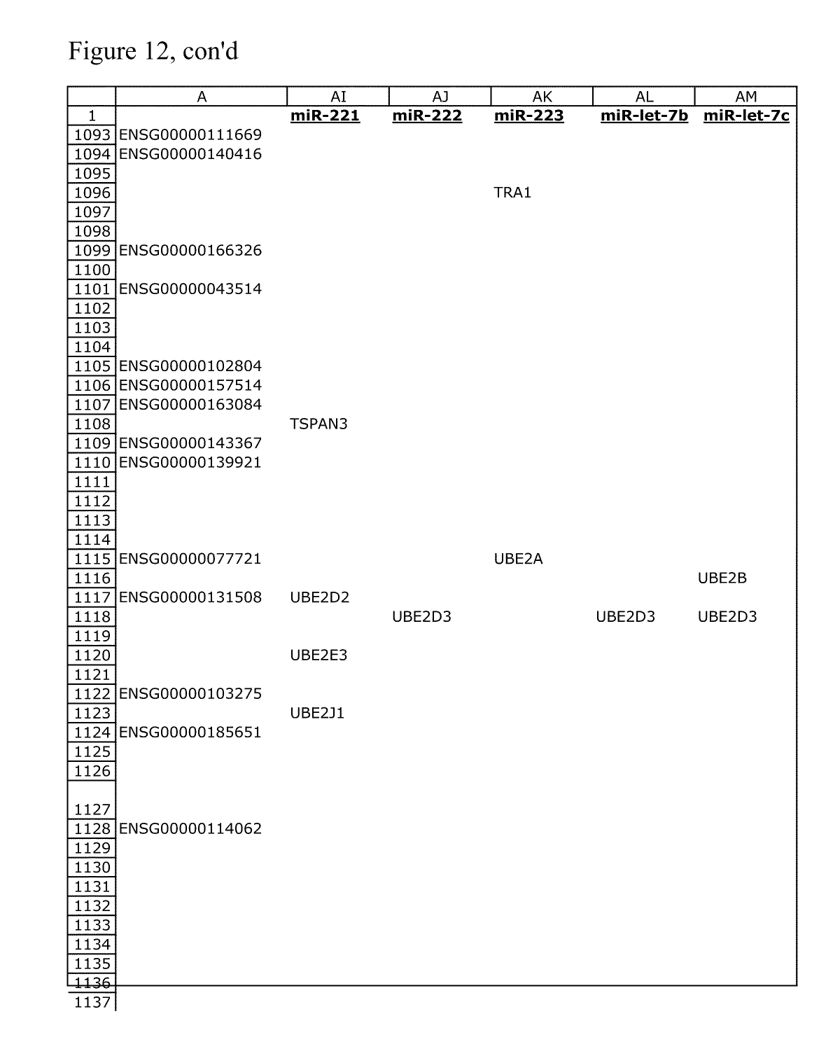

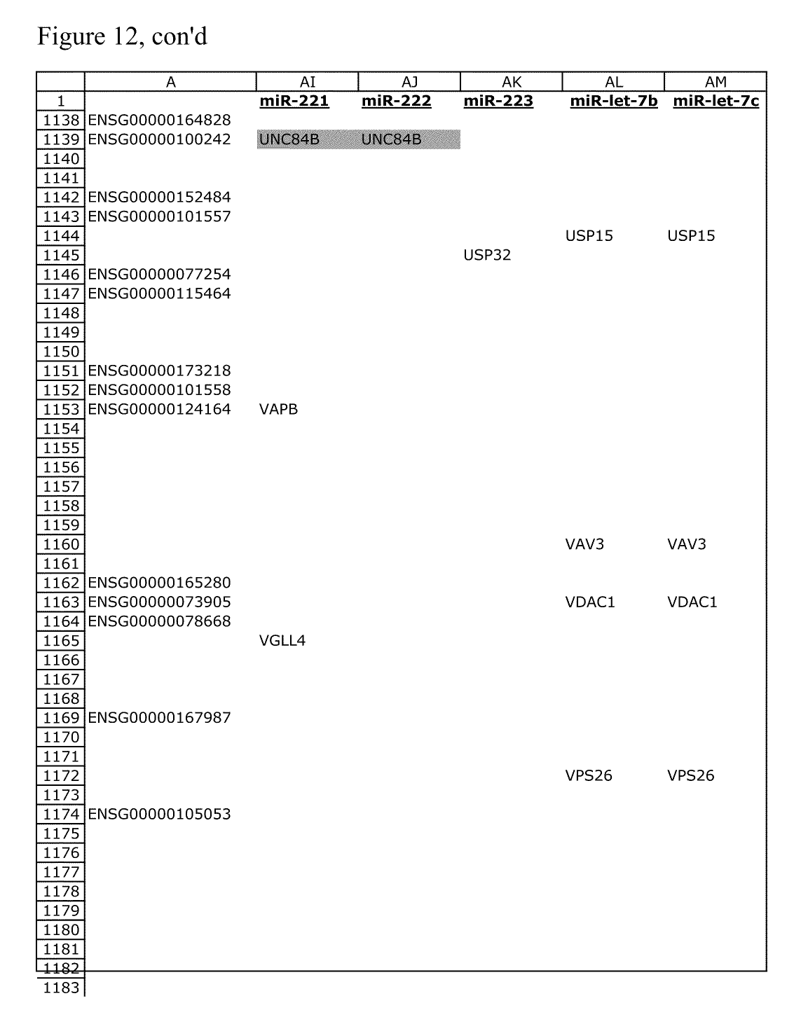

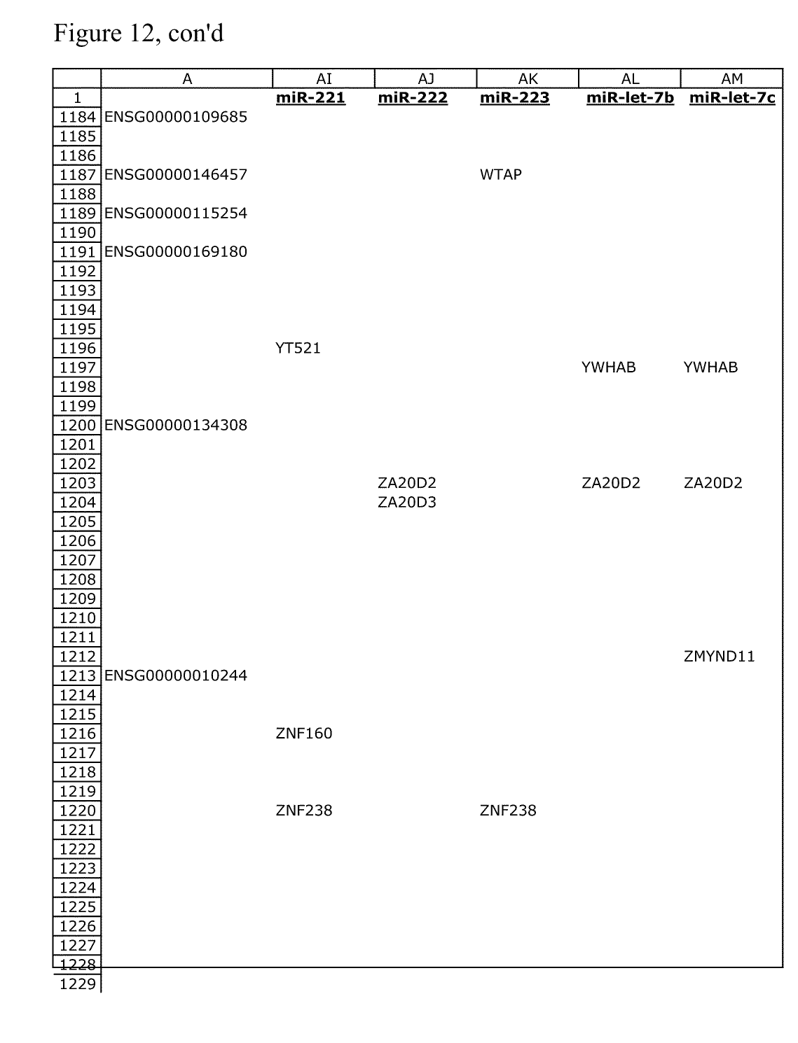

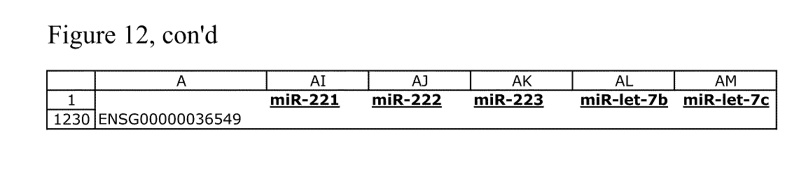

Combining the prior HSPC mRNA expression data sets (8) with the miRNA expression data generated in the current study, along with the miRNA target predictions (9-11), the inventors mapped each of the 33 HE-miRNAs to its multiple predicted target mRNA transcripts expressed in CD34+ cells (from both PBSC and BM). mRNAs (16.7%) expressed by CD34+ HSPCs (HE-mRNAs) from both PBSC and BM are predicted to be regulated by at least one HE-miRNA (i.e., 1,230 of the 7354 mRNAs expressed by CD34+ cells potentially were HE-miRNA-regulated; FIGS. 9 and 10), including several mRNAs thought to be associated with hematopoietic differentiation (Table 2). These in silico data allowed the inventors to formulate a bioinformatic model predicting which HE-miRNAs may control hematopoietic differentiation and at which hematopoietic stages they may act (FIG. 1).

TABLE 2
HSPC-expressed mRNAs associated with hematopoietic differentiation and predicted to be regulated by HE-miRNAs
Erythroid*

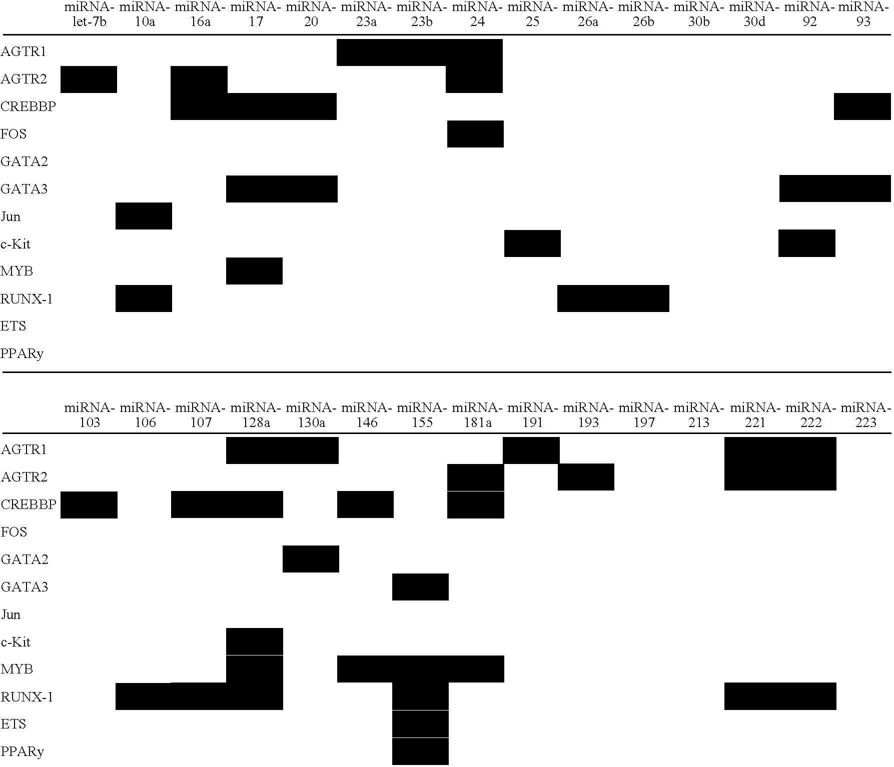

Myeloid

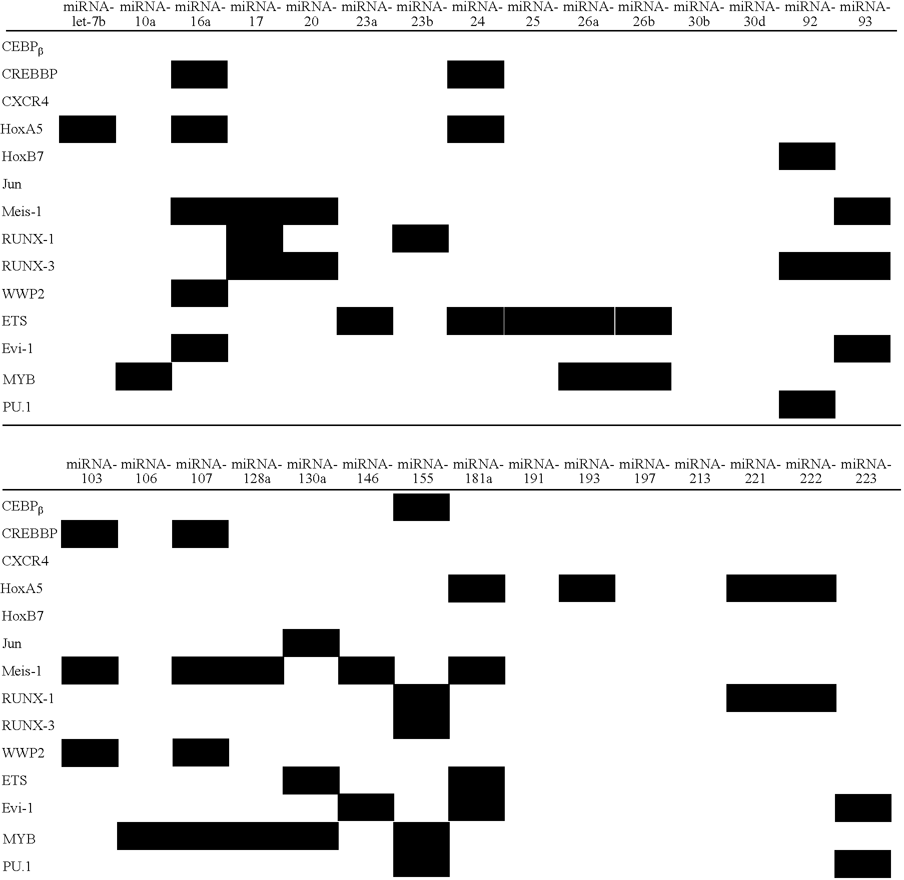

Lymphoid

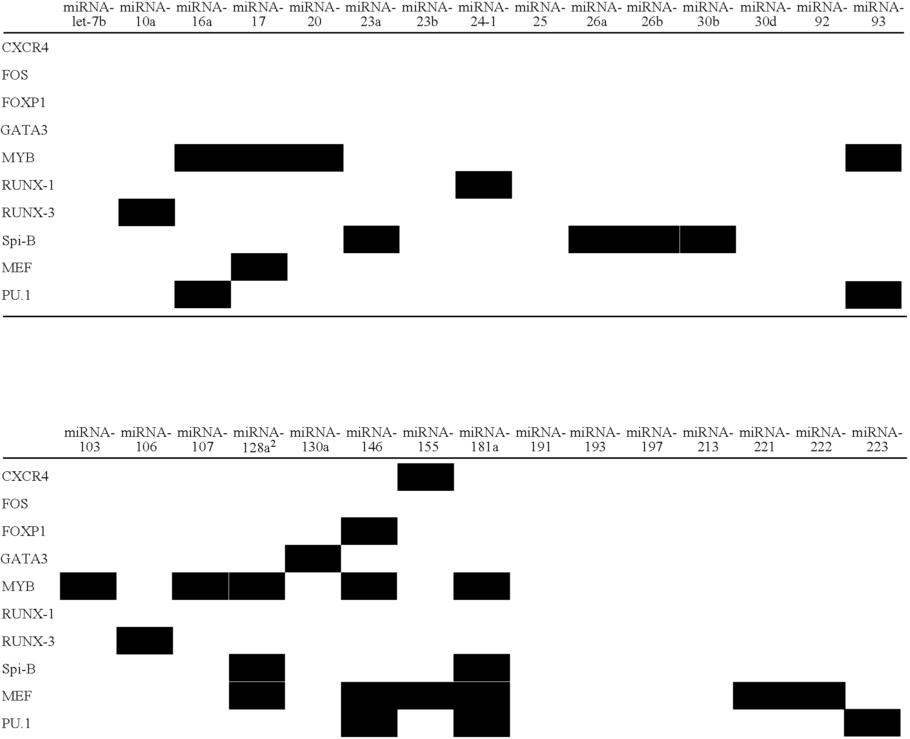

miRNAs in bold are those that, based on the number of predicted target rnRNAs they hit as well as on the strength of association to each category based on the annotation, the inventors predict will be the major regulators of each particular category of differentiation. mRNAs are grouped into categories reflecting their roles in lymphoid, myeloid, and/or erythroid differentiation.

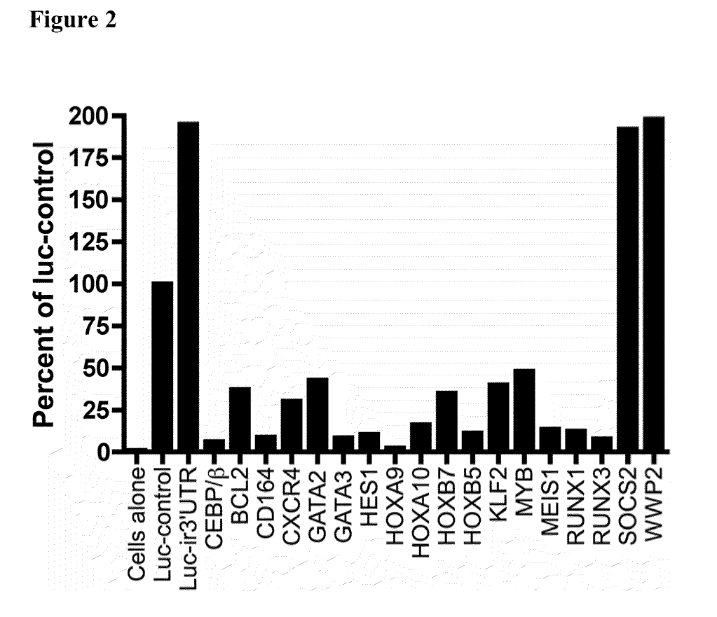

HE-miRNAs Regulate Hematopoietic Differentiation-Associated mRNAs in CD34+ cells. Of the HSPC-expressed mRNAs putatively targeted by HE-miRNAs, 18 mRNAs were selected, because they were either well established and/or annotated to have a role in regulation of hematopoietic differentiation. For each of these selected “hematopoietic differentiation-associated” mRNAs, the 3′ UTR was cloned into the 3.1-Luc expression vector. K562 cells were transduced with each chimeric luc-3′ UTR mRNA. Luc activity in the transduced K562 cells for the following mRNAs was reduced by 52-92% as compared with the luc-positive control: BCL2, C/EBP-beta, CXCR4, GATA2, GATA3, HES1, HOXA9, HOXA10, HOXB5, HOXB7, KLF2, MEIS1, MYB, AML1/RUNX1, and RUNX3 (FIG. 2). However, luc activity of the SOCS2 and WWP2 constructs was increased compared with the luc-positive control; these two activities were identical to that of a chimeric mRNA (Luc-nmiR) containing a 3′UTR devoid of predicted miRNA-binding sites, which (for unknown reasons) resulted in almost twice the control luc activity. Thus, the predictions of targets of the HE-miRNA data resulted in a false-positive rate of 10.5%, considerably lower than the 25-40% false-positive rate postulated by Bentwich (12).

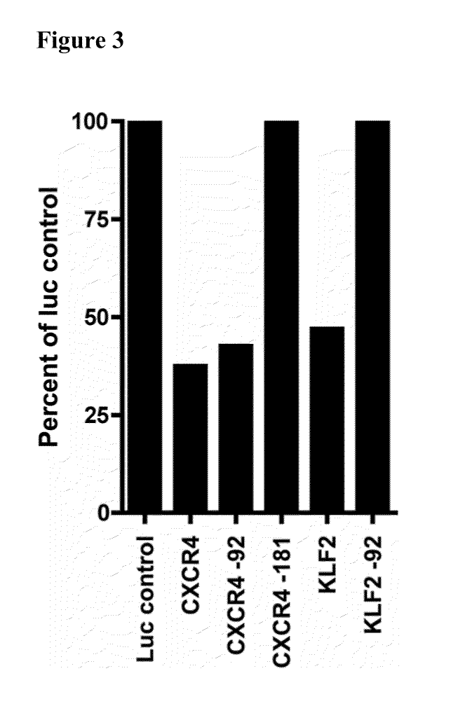

The inventors further characterized HE-miRNA translational control of two of the hematopoietic differentiation-associated mRNAs, by deleting the 21- to 25-nt miRNA binding sites from the 3′ UTRs in the chimeric luc reporter constructs (FIG. 3). Deletion of the miRNA-92-binding site from the KLF2 3′ UTR restored luc expression, demonstrating that miRNA-92 regulates KLF2 in hematopoietic cells. Similarly, deletion of the binding site for miRNA-181 from the CXCR4 3′ UTR resulted in restoration of luc activity, whereas deletion of the miRNA-92-binding site from the CXCR4 3′UTR did not significantly change luc activity, indicating that CXCR4 is specifically modulated by mlRNA-181.

miRNA-155 Controls Myeloid and Erythroid Differentiation.

miRNA-155 was predicted to block hematopoietic differentiation along more than one lineage (FIG. 1), because it was predicted to block protein expression of CREBBP, CXCR4, JUN, MEIS1, PU.1, AGTR1, AGTRII, FOS, and GATA3. Consistent with this Prediction, <I% of miRNA-155-transduced K562 cells, as compared with about 65% of control cells, were treated with hemin differentiated toward erythrocytes (data not shown). In addition, 57%±3% of miRNA-155-transduced K562 cells expressed CD41 (mean fluorescence intensity (MFI)=11.5±1.2), in comparison with 78±5% of control K562 cells (MFI=32.8±3.5) treated with IDB-expressed CD41 (data not shown). Both the decrease in percentage of cells positive for CD41, as well as the about 3-fold decrease in MFI, are indicative of a large decrease in K562 cell megakaryocytic differentiation (13-15). K562 cells transduced with miRNA-155 proliferated at a similar rate to those transduced with control vector (data not shown), indicating that the observed lack of differentiation was not due to cell cycle arrest or decreased cell growth rate.

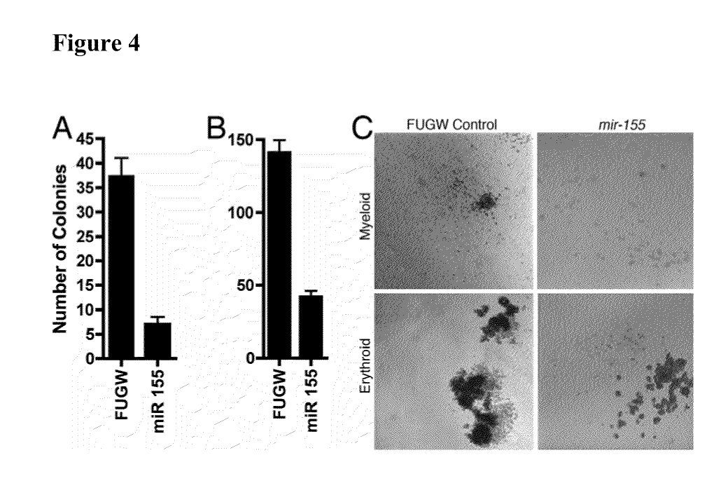

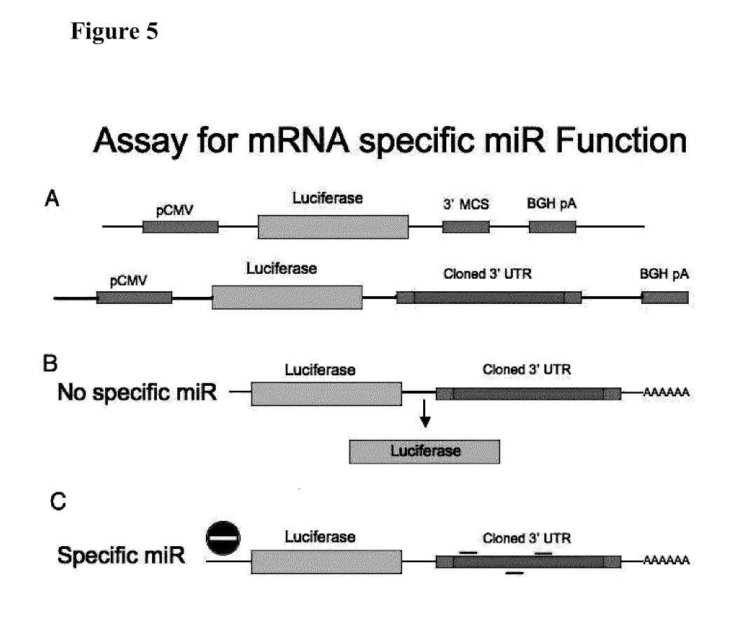

Normal human PBSC CD34+ cells were transduced with FUG W-miRNA-155, and assayed for hematopoietic colony formation. miRNA-155-transduced cells generated 5-fold fewer myeloid and 3-fold fewer erythroid colonies. Furthermore, the colonies generated by miRNA-155-transduced cells were much smaller than controls (FIG. 4). These data demonstrate that miRNA-155 can control both myeloid and erythroid differentiation of human HSPCs.

### Discussion

The present inventors used bioinformatic techniques to combine human HSPC miRNA expression with mRNA expression to begin to define the roles that HE-miRNAs play in human hematopoiesis. The bioinformatic and functional analyses of HE-miRNA action indicate that multiple miRNAs, including miRNA-17, -24, -146, -155, -128, and -181, may hold early hematopoietic cells at an early stem-progenitor stage, blocking their differentiation to more mature cells. miRNA-16, -103, and -107 may block differentiation of later progenitor cells; miRNA-221, -222, and -223 most likely act to control terminal stages of hematopoietic differentiation (FIG. 1). As an initial validation of the in silico predictions, the inventors demonstrate that miRNA-155 is a potent inhibitor of HSPC differentiation.

In a prior mRNA expression study (8), the inventors demonstrated that many genes known to be associated with differentiation of HSPCs (including RUNX1, MEIS1, ETS1, and PU.1) are equivalently expressed at the mRNA level in both the HSC-enriched and the more differentiated HPC-enriched populations. The Transcriptome Interaction Database predicts that about 16.7% of the mRNAs expressed by HSPCs may be regulated at the translational level by the 33 HE-miRNAs. Taking into account the consensus that such predictions likely contains 25% false-positives (12), it is estimated that 10.5% of the HSPC-expressed mRNAs are controlled by HE-miRNAs. This is in concordance with a recent study (16), which compared a limited proteome of HSPCs to various transcriptome studies (including the inventors' own study; ref 8) and found that the expression of about 15% of proteins were underestimated by their mRNA expression levels.

Based on their predicted mRNA targets, it was found that the HE-miRNAs annotated as having hematopoietic function fall into three categories (FIG. 1): (i) those which might inhibit differentiation of all hematopoietic lineages (including B/T/NK lymphoid) by regulating molecules critical to very early steps in hematopoiesis (miRNA-181 and -128); (ii) those that might act in differentiation of (a) lymphoid (miRNA-146); (b) granulocytic/monocytic/erythroid/megakaryocytic/B-lymphoid (miRNA-155, -24, and -17), or (c) granulocytic/monocytic/B-lymphoid (miRNA-16, -103, and -107); and (iii) in combination with other published studies (3-5), those that act only on one specific lineage of blood cells (miRNA-221 and -222 in erythroid development and miRNA-223 in granulocytic development).

The model and specific in silico predictions were validated by testing HE-miRNA translational repression of selected predicted target mRNAs. Of the 18 hematopoietic differentiation-associated mRNAs tested in the model system, it was found that protein expression from 16 were down-regulated greatly in K562 cells expressing the implicated HE-miRNAs. The translational repression that was observed was generally quantitatively greater than that of other past miRNA functional studies, where a 40-60% miRNA-mediated decrease in protein expression has proven to be biologically relevant (4,5). Other studies, although correct, may greatly underestimate the translational repression mediated by HE-miRNAs, because of several factors. First, the present inventors extensively optimized their assays to quantify differences in protein expression. Because the CMV promoters used in these plasmids are such strong initiators of transcription, the present inventors strove for a “low-level” transfection in an attempt to minimize the number of plasmids entering individual cells. Further, the present inventors added the additional control of a chimeric-mRNA containing a 3′ UTR having no predicted miRNA-binding sites. This control vector resulted in twice the amount of luc expression compared with the control vector containing no 3′UTR sequence. It may be that using only a luc alone leads to a major underestimation of miRNA action, and the luc-nmiR may be a much more biologically relevant control, because it mimics the natural structure of mRNAs. To test more directly which miRNAs mediate the observed decrease in protein expression, for two targeted mRNAs, the present inventors produced chimeric mRNAs with 3′ UTRs that had specific deletions of only the predicted miRNA-binding site. In both cases, deletion of miRNA-binding sites completely restored protein expression.

To begin to confirm the biological effects of HE-miRNAs, miRNA-155 was studied because it was predicted to target multiple hematopoietic differentiation-associated molecules, including C/EBP-beta, CREBBP, JUN MEIS1, PU.1, AGTR1, AGTR2, and FOS. In addition, it has been reported that miRNA-155 is overexpressed in undifferentiated CD34+/CD38− acute myeloid leukemia (AML) stem cells and that ectopic over-expression of miRNA-155 in AML cells blocks differentiationli. In the inventors' studies, transduction of cells with miRNA-155 followed by treatment with each of two chemical inducers of differentiation showed that miRNA-155 decreased both erythroid and megakaryocytic differentiation in K562 cell line models of human hematopoiesis. More important, miRNA-155-transduced normal human CD34+ cells generated far fewer (and smaller) myeloid and erythroid colonies than controls. Thus, miRNA-155 negatively regulates normal myelopoiesis and erthryopoiesis.

Most of the cells within the CD34+ population that were studied have differing degrees of self-renewal capacity. In that light, perhaps it is not surprising that the present inventors did not identify any HE-miRNAs that targeted mRNAs and are thought to be involved in self-renewal, such as HOX-B4 (18) and BMI-1 (19, 20).

Three other studies have begun to examine the functional role of miRNAs in the differentiation of specific aspects of hematopoiesis. Fazi et al. (5) showed that murine mmu-miRNA-223 up-regulates mouse granulopoiesis by blocking protein expression of NFI-A, a factor that represses granulopoiesis. The myeloid regulator C/EBP-alpha drives miRNA-223 expression, thereby removing NFI-A mediated differentiation repression. The model of human HE-miRNA regulation of hematopoiesis agrees with this finding in that the Transcriptome Interaction Database predicts that human hsa-mir-223 regulates human NFI-A translation.

Felli et al. (4) showed that human miRNA-221 and -222 block erythropoiesis via translational repression of c-KIT. The inventors' findings strongly support that miRNA-221 and -222 block erythropoiesis. Not only do the in silico studies predict that miRNA-221 and -222 block c-KIT translation, but they also predict that they may block CREBBP, FOS, and PPAR-gamma (additional molecules highly associated with erythroid differentiation) (21-24). miRNA-221 and -222 also may be more generalized inhibitors of hematopoietic differentiation, because the data (Table 2) predict that they also may regulate myeloid differentiation-associated molecules (FIG. 2; MEIS1 and ETS1, both proteins shown to be involved in myeloid development (25, 26), and FOS, which has been implicated in lymphopoiesis; ref 27 and unpublished data). Consistent with this, Felli et at (4) found that overexpression of miRNA-221 and -222 inhibited engraftment of human CD34+ cells in NOD-scid mice, pointing to a more generalized role in differentiation.

Chen et al. (3) studied the role of mmu-mir-142, -181, and -223 in mouse hematopoiesis and found that ectopic overexpression of miRNA-181 in mouse HSPCs resulted in an increase in B-lymphopoiesis, and miRNA-142 and -223 resulted in small but significant increases in T-lymphopoiesis. Unfortunately, the relevant target mRNAs were not determined in that early study. It is important to note that although miRNA-181 is strongly expressed in human CD34+ cells, in the murine system, it seems to be expressed selectively in B lymphocytes. In fact, in human BM, miRNA-181 is expressed more weakly than miRNA-146, an miRNA that Chen et al. (3) finds expressed strongly in all mouse hematopoietic tissues. In addition, the present inventors found that miRNA-142 is not expressed in human HSPCs. Thus, these HE-miRNAs may be expressed and act differently in human hematopoiesis than in mouse hematopoiesis; this interpretation is substantiated by the finding that the same types of human and mouse hematopoietic cells show large differences in miRNA expression (28). Although the predictive data are consistent with a possible outcome that miRNA-181 may affect B lymphoid development, the in silico model indicates that miRNA-181 and miRNA-146 might block differentiation very early in human lymphopoiesis.

This in silico bioinformatic study combines global miRNA and mRNA expression data into a model for exploring the action of miRNAs within a cellular system. The present inventors validated the in silico predictions in two ways: (i) The present inventors showed that 16 of 18 “hematopoietic” mRNAs are controlled by HE-miRNAs and (ii) the inventors functionally validated miRNA-155 as potent controller of normal human myelopoiesis and erythropoieis. A skilled artisan will recognize that these studies will serve to guide the work of other researchers examining the role of miRNAs in hematopoiesis. In certain emodiments, the present invention has utility in models in which many of the genetic components associated with hematopoietic differentiation are expressed at an early time point by undifferentiated HSPCs but are controlled by HE-miRNA-mediated repression of protein translation until differentiation is initiated.

### Methods

Cell Samples. Human CD34+ cells. Cryopreserved cadavaric BM and PBSC CD34+ cells from normal adult donors were obtained from the National Heart, Lung, and Blood Institute Program of Excellence in Gene Therapy, Hematopoietic Cell Processing Core (Fred Hutchison Cancer Center). Each BM sample was a pool of cells from two donors. Each PBSC sample was a pool from three to five donors. CD34+ cells were thawed and washed twice with PBS (8).

Cell lines. HEK 293T, Hal-01, HL60, K562, KG1, KG1a, MV-4-1, REH, TF1, and TF1a cell lines were purchased from the American Type Culture Collection (ATCC; Manassas, Va.). A different K562 cell subline, used in the cellular differentiation studies, was provided by Michael McDevitt (The Johns Hopkins University) (29). All cells were cultured by following instructions provided by the ATCC.

miRNA Expression Microarrays. CD34+ cells were disrupted with TRIzol Reagent (15596-018; Invitrogen. Carlsbad, Calif.). Five micrograms of total RNA obtained from the TRIzol preparations were analyzed with miRNA chips (30). Standard microarray methods were used for normalization and statistical analysis of the miRNA expression data. Statistical analyses were carried out in GeneSpring 7.0 (Silicon Genetics/Agilent Technologies, Palo Alto, Calif.) to determine the RFU value fora 95% confidence cutoff for miRNA expression. Significantly expressed miRNAs were identified by using the GeneSpring Cross-Gene Error Model (8). Thus, for each set of samples (PBSC or BM,) values from individual samples were normalized and averaged, then expressed miRNAs were identified as those with a P<0.05 of expression over background (i.e., the weighted averaged values of all chip measurements). Then, to perform intersection analysis, the inventors determined those microRNAs expressed by both PBSC and BM CD34+ samples at the P<0.05 significance level (Student's t test).

Bioinformatic Integration of mRNA and miRNA Expression Data. miRNA expression data, mRNA expression data (determined in ref. 8), and miRNA target prediction data (from Lewis et al., refs. 10 and 11; www.targetscan.org), Sloan-Kettering Cancer Center Human MicroRNA Targets Database (www.microRNA.org; ref. 9) and miRBase Targets (microrna.sanger.ac.uk/targets/v3) were combined in a Filemaker Pro 7.0 (Filemaker Corp., Santa Clara, Calif.) relational database, which was called the “Transcriptome Interaction Database.” Individual data tables consisted of miRNA expression from each individual sample, PBSC CD34+ cell mRNA expression data, BM CD34+ cell mRNA expression data, and miRNA targets predicted by each of the three databases. This integrated database allows the inventors to interrogate directly the predicted interactions of HE-miRNAs with HSPC-expressed mRNAs. Regulated mRNAs were annotated for possible hematopoietic function through both manual and electronic annotation. Manual annotation consisted of the authors scanning the lists of mRNA targets for those with known hematopoietic function. Electronic annotation consisted of querying the PubMed (www.ncbi.nlm.nih.gov/entrez/query.fcgi?holding=jhumlibfft_ndi) and Gene Ontology (www.geneontology.org) databases with search terms of the “gene common name” and terms including “hematopoiesis,” “myeloid,” “erythroid,” “blood,” etc. After compiling the list of candidate hematopoietic differentiation-associated mRNAs, PubMed searches were performed, and peer-reviewed publications were reviewed to assure that these mRNAs actually had been implicated in hematopoietic differentiation. At least two articles, each directly demonstrating either that the mRNA of interest is involved in differentiation or critical to a molecular pathway of a known hematopoietic regulator, were required for the mRNA to be scored as hematopoietic differentiation-associated. In addition, the present inventors also classified certain genes expressed in the HSC-enriched population (8) as hematopoietic differentiation-associated.

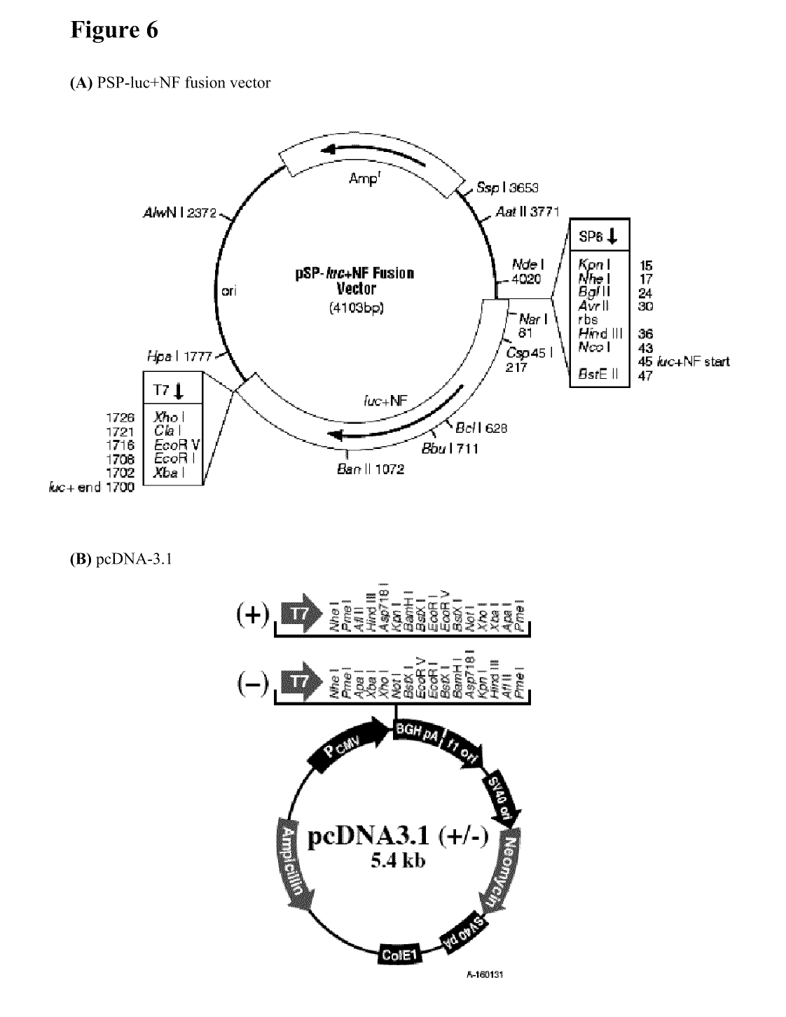

Functional Analysis of Expressed HE-miRNAs. Plasmids and constructs: Luciferase(luc)-3′ UTR reporter plasmids. The luc-coding sequence and 3′ multiple-cloning-site (MCS) from the promoterless pSP-luc+NF fusion vector (E4471; Promega, Madison, Wis.) were subcloned into the NheI and XhoI sites of the pcDNA3.1(+) expression vector (V79020; Invitrogen). The resulting plasmid (Luc-3′ UTR) construct contains a strong CMV promoter driving a luc-expression cassette, followed by a 3′ MCS. The 3′ UTR sequences from several hematopoietic differentiation-associated mRNAs were isolated by RT-PCR with primers containing restriction enzyme linkers. These 3′ UTR PCR fragments were cloned into the 3′ MCS of the Luc-3′ UTR reporter construct. The resulting chimeric mRNAs, containing luc and the 3′ UTR of the mRNA of interest, allowed for rapid functional analysis of miRNA translational control of mRNAs of interest (FIG. 6).

In addition, for certain of the luc-3′ UTR reporter plasmids, the present inventors produced variant constructs, in which specific miRNA-binding sites were deleted from the 3′ UTR (Luc-3′ UTR-DL constructs). Approximately 60-mer PCR primers, consisting of the 20-nucleotide (nt) sequence on either side of the miRNA-binding sites plus an intervening 25-bp spacer containing a Pad site, were generated (http://www.pnas.org/cgi/content/full/0610983104/DC1) and then used in full plasmid PCR (QuikChange XL Kit 200516; Stratagene, La Jolla, Calif.). Briefly, these primers span the deletion site but replace the sequence to be deleted with a unique restriction enzyme site that allows for rapid screening of clones with the deletion. The entire mutant plasmid then is produced via PCR, lacking the miRNA-binding site, as described in the manufacturer's protocol.

miRNA expression constructs. The hsa-mir-155 gene was cloned from K562 cell genomic DNA via PCR by using primers containing restriction enzyme linkers that would allow cloning into either the pcDNA3.1(+) expression vector (V79020; Invitrogen) or the FUGW lentiviral expression vector (31). The inventors used vectors containing strong PolII promoters, because miRNA expression and processing by Drosha, Exportin V, and Dicer seem to be much more reliable with longer 5′-capped-mRNA sequences produced by PolII, rather than shorter RNAs produced by PolIII promoters (32). The fragments produced by PCR contained the miRNA precursor (70-120 nt) flanked on both the 5′ and 3′ sides by 250 nt of genomic sequence, resulting in inserts of 570-620 nt.

Expression of miRNA precursor and mature sequences were tested by Lipofectamine (11668-019; Invitrogen) transfection of 293T cells with these expression constructs. Small RNAs were isolated from the transfected cells (MirVana miRNA Isolation Kit 1560; Ambion, Austin, Tex.). Precursor and mature miRNA expression was determined by miRNA RT-PCR (mirVana qRT-PCR miRNA Detection Kit 1158; Ambion; using specific mirVana qRT-PCR Primer Sets; Ambion) and/or by Northern blotting with ³²P-radiolabeled probes specific for the mature miRNA sequence (mirVana miRNA Probe Construction Kit 1550; Ambion).

HE-miRNA Translational Control of Hematopoietic Differentiation-Associated mRNAs Expressed in HSPCs. The inventors determined the miRNA expression of 10 hematopoietic cell lines and found that the K562 cell line expresses many HE-miRNAs (FIG. 13). Luc-3′ UTR and Luc-3′ UTR-DL constructs were electroporated into K562 cells (Nucleofector V Kit VCA-1003; Amaxa, Cologne, Germany; Nucleofector II Device AAD-1001; Amaxa). Control plasmids of backbone Luc-3.1 and Luc-nmiR (containing a 3′ UTR with no predicted miRNA-binding sites) controls also were used. Each electroporation included 100 ng of beta-galactosidase (beta-gal)-3.1 plasmid to allow normalization of luc activity. Three days after transfection, cells were lysed (Passive Lysis Buffer E194A; Promega), and luminescence was measured to quantify luc protein expression (Model 1500; Analytical Luminescence Laboratories, San Diego, Calif.; Luciferase Assay System E1501; Promega) and beta-gal expression (Galactolight Plus Kit; Tropix, Bedford, Mass.). Relative beta-gal units were used to generate individual normalization factors to standardize the Relative Luc Units for each sample to the Luc-nmiR-positive control.

Cellular Differentiation Assays. K562 cells were transduced with FUGW-miRNA-155 or FUGW-parental control (i.e., empty vector) lentivirus at a multiplicity of infection of 5. Each of these vectors contains a GFP cassette driven from a second promoter (31). Cells were cultured for 2 days and then checked for GFP expression, indicating lentiviral transduction. Cells then were washed with PBS and cultured incomplete growth media containing ingenol 3,20-dibenzoate (IDB, 1 μg/ml; Sigma) or hemin (50 μM; Sigma). On day 4, IDB-treated cells were assayed by monoclonal antibody staining and flow cytometry for CD41 (Becton Dickinson, Franklin Lake, N.J.) expression, indicating megakaryocytic differentiation (13-15). Hemin-treated cells were assayed similarly on day 6 for benzidine staining, indicating erythroid differentiation (33). Human PBSC CD34⁺ cells were transduced with miRNA-155 or control lentivirus and assayed by standard colony-forming unit (cfu) assays for myeloid and erythroid differentiation (34).

### Additional Methods

Luciferase(luc)-3′ UTR Reporter Plasmids. The luc coding sequence and 3′ multiple-cloning-site (MCS) from the promoterless pSP-luc+NF fusion vector (E4471; Promega, Madison, Wis.) were subcloned into the NheI and XhoI sites of the pcDNA 3.1(+) expression vector (V79020; Invitrogen, Carlsbad, Calif.). The resulting plasmid (Luc-3′ UTR) construct contains a strong CMV promotor driving a luc expression cassette, followed by a 3′ MCS. (FIG. 6) The 3′ MSC contains the restriction sites XbaI-EcoRI-EcoRV-ClaI-XhoI. The 3′ UTR sequences from several hematopoietic differentiation-associated mRNAs were isolated by RT-PCR with primers containing restriction enzyme linkers. These 3′ UTR PCR fragments were cloned into the 3′ MCS of the Luc-3′ UTR reporter construct. The resulting chimeric mRNAs, containing luc and the 3′ UTR of the mRNA of interest, allowed for rapid functional analysis of miRNA translational control of mRNAs of interest (FIG. 6)

Primer sequences: The name of the mRNA is followed by the (size of the insert) in base pairs. L and R represent the forward (left) and reverse (right) primers used for PCR, each followed by the (restriction enzyme) used to clone the insert into the Luc-3′ UTR reporter construct. Within the sequences the restriction site is shown in capital letters. Each prime also contains a 6-bp extension to act as a GC clamp and as a spacer for restriction enzyme binding.

BCL-2 3′ UTR Primers (1,483 bp)
L: (EcoRI) gcattgGAATTCagtcaacatgcctgccccaaacaaatatgc
R: (XhoI) ccgatacCTCGAGgttttacatgtaattccatagacaggggt
CD164 3′ UTR Primers (2,307 bp)
L: (ClaI) gcattgATCGATacagacccattgaattaataaggactggtgattc
R: (XhoI) cgatacCTCGAGgtcagtttggtacatgatacagattggttttgcag
C/EBP-b 3′ UTR primers (5,96 bp)
L: (EcoRI) gcattgGAATTCcgcggcccccgcgcgcgtccccctgccggc
R: (XhoI) cgatacCTCGAGaaccattctcaaaaaaaagtttattaaaag
CXCR4 3′ UTR primers (5,19 bp)
L: (EcoRI) gcattgGAATTCcacagatgtaaaagacttttttttatacga
R: (XhoI) cgatacCTCGAGaagtttaacatgtacttttattaacaactt
GATA-2 3′ UTR primers (1,589 bp)
L: (EcoRI) gcattgGAATTCggaacagatggacgtcgaggaccgggcact
R: (XhoI) cgatacCTCGAGacaaaaagtattttattattcttaacagta
GATA-3 3′ UTR primers (475 bp)
L: (EcoRI) gcattgGAATTCagccctgctcgatgctcacagggcccccag
R: (XhoI) cgatacCTCGAGaatgcatcaaacaactgtggccagtgaaag
Hes-1 3′ UTR primers (295 bp)
L: (EcoRI) gcattgGAATTCctccggactctaaacaggaacttgaatact
R: (XhoI) cgatacCTCGAGctcaaataaacttccccaaaggaagatatt
Hox A5 3′ UTR primers (284 bp)
L: (EcoRI) gcattgGAATTCgtatctgagcgtttaaagtactgagcagta
R: (XhoI) cgatacCTCGAGgttatagttacttcaagtaacacagcttgc
Hox A9 3′ UTR primers (1,165 bp)
L: (ClaI) gcattgATCGATtgccatttgggcttatttagaaaaaagggt
R: (XhoI) cgatacCTCGAGagaaaagaatcaatatattttatttggcaa
Hox A10 3′ UTR primers (1,298 bp)
L: (EcoRI) gcattgGAATTCtgaatctccaggcgacgcggttttttcact
R: (XhoI) cgatacCTCGAGgcttcattccacagcttttattctataaga
Hox B5 3′ UTR primers (951 bp)
L: (EcoRI) gcattgGAATTCgcccgcccagaggagcccagcggcccaaga
R: (XhoI) cgatacCTCGAGtggaagtcaaataaatgtttcccccactcc
Hox B7 3′ UTR primers (586 bp)
L: (EcoRI) gcattgGAATTCgggatggagaaagggcagaggaag
R: (XhoI) cgatacCTCGAGtccatcctttagatacacacagaatgtaaaaggg
KLF2 3′ UTR primers (498 bp)
L: (EcoRI) gcatgcGAATTCaaccgggccgggcacagc
R: (XhoI) cgatgcCTCGAGtcttataaaaacgaaccaggtagccc
LMO2 3′ UTR primers (778 bp)
L: (EcoRI) gcattgGAATTCgcccgagtccccgggcatctttggggaggt
R: (XhoI) cgatacCTCGAGcagtctgtcatttttattaagcctgcagag
MEIS-1 3′ UTR primers (1,500 bp)
L: (EcoRI) gcattgGAATTCccttcatctagttaaccaatcgcaaagcaa
R: (XhoI) cgatacCTCGAGgacaaaaataatcttgtttttattttagat
Myb 3′ UTR primers (1,157 bp)
L: (EcoRI) gcattgGAATTCccagaaaagcattatggttttcagaacac
R: (XhoI) cgatacCTCGAGtgctacaaggcagtaagtacaccgtc
RUNX-1 3′ UTR primers (4,331 bp)
L: (EcoRI) gcattgGAATTCggcgccaggcctggcccggctgggccccgc
R: (ClaI) gcattgATCGATatggtgtatgtacttgtcaaactgtttatttgcc
RUNX-3 3′ UTR primers (1,062 bp)
L: (EcoRI) gcattgGAATTCccgccctggtggactcctcccgctggaggc
R: (ClaI) gcattgATCGATactatttgctttcagagcacaaaacaggtt
SOCS-2 3′ UTR primers (1,000 bp)
L: (EcoRI) gcattgGAATTCatgtctcacatagagtatctccgaatgcag
R: (XhoI) cgatacCTCGAGcagtagaagggagacaactgtttatgtcca
WWP-2 3′ UTR primers (1,800 bp)
L: (EcoRI) gcattgGAATTCccgaggccgcccctcccacgccccccagcg
R: (XhoI) cgatacCTCGAGacagctgctgattgtttattgcacaagaaa

miRNA Knockout Luciferase(luc)-3′ UTR Reporter Plasmids. In addition, for certain of the luc-3′UTR reporter plasmids, the inventors produced variant constructs, in which specific miRNA-binding sites were deleted from the 3′ UTR (Luc-3′ UTR-DL constructs). Approximately 60-mer PCR primers, consisting of the 20-nucleotide (nt) sequence on either side of the miRNA-binding sites plus an intervening 20-bp spacer containing a PacI site, were generated and then used in full-plasmid PCR (QuikChange XL Kit 200516; Stratagene, La Jolla, Calif.). Briefly, these primers span the deletion site but replace the sequence to be deleted with a unique restriction enzyme site that allows for rapid screening of clones with the deletion. The entire mutant plasmid is then produced via PCR, lacking the miRNA binding site, as described in the manufacturer's protocol.

Primer sequences: The name of the primer consists of the mRNA 3′ UTR name and the predicted miRNA binding site deleted from the 3′ UTR sequence. Only the forward primer sequence is shown, because the reverse is the antiparallel sequence of the forward primer. The PacI spacer is shown in blue.

CXCR4 3′ UTR mir-92 deletion primers
ttcctgttcttaagacgtgaacagtaTTAATTAAagctgtaaccaaagcc
caaagtggta
CXCR4 3′ UTR mir-181 deletion primers
gactgtagaaaagggaactgacagtaTTAATTAAagctgtgtaaagctag
aaatgatccc
KLF2-82 3′ UTR mir-92 deletion primers
CGAGTTTTGTTTTTCAAAATacagtaTTAATTAAagctgtATTTAAGTG
GCATCTTCTCT

Cloning of the hsa-mir-155 Gene. The hsa-mir-155 gene was cloned from K562 cell genomic DNA, via PCR by using primers containing restriction enzyme linkers that would allow cloning into either the pcDNA3.1(+) expression vector (V79020; Invitrogen, Carlsbad, Calif.) or the FUGW lentiviral expression vector (1). The inventors used vectors containing strong Pol II promotors, since miRNA expression and processing by Drosha, Exportin V, and Dicer seem to be much more reliable using longer 5′-capped-mRNA sequences produced by Pol II, rather than shorter RNAs produced by Pol III promotors (2). The fragments produced by PCR contained the miRNA precursor (70-120 nt) flanked on both the 5′ and 3′ sides by 250 nt of genomic sequence, resulting in inserts of 570 to 620 nt.

### Primer Sequences:

L: (EcoRI) cgatcgGAATTCaaatggactattttcctttcagattta
cta
R: (NheI) gctatgGCTAGCtaaataaccttactttcaaaacttagt
tta

1. Yu X, Zhan X, D'Costa J, Tanavde V M, Ye Z, Peng T, Malehorn M T, Yang X, Civin C I, Cheng L (2003) Mol Ther 7:827-838.
2. Bartel D P (2004) Cell 116:281-297.

### Example 2

### Abstracts

### CD34+ Hematopoietic Stem-Progenitor Cell MicroRNA Expression and Function: The miR Circuit Diagram of Differentiation Control.

Since lin-4, the founding member of the class, was described; microRNAs (miRs) have become a recently realized class of epigenetic elements which modify translation of mRNA to protein, and which also may result in gene silencing through chromatin remodeling. First discovered in C. elegans, miRs have been identified in numerous other organisms including drosophila, rat, mouse, and humans. To date, they have been shown to control cellular metabolism, differentiation and development. Even more importantly aberrant expression of miRs and deletion of miRs are highly associated with the development of various cancers. To better understand the role of miRs in normal hematopoiesis the inventors have determined the microRNA expression profile of primary normal human PBSC and bone marrow CD34+ cells. The inventors have combined this data with the extensive mRNA expression data obtained from CD34+ HSPC, CD34+/CD38−ILin−HSC-enriched, and CD34+/CD38+/Lin+HPC-enriched populations in a previous study. (Cancer Research 644344) Combining these two datasets into one integrated database has allowed the inventors to intricately examine the global interaction of HSPC mRNAs and microRNAs, and to also predict which miRs are involved with differentiation of the hematopoietic system. These findings offer promising new targets for the study of stem cell differentiation and targets for genetic therapies of hematopoietic stem cells.

### CD34+ HSPC MicroRNA Expression and Regulation of Hematopoiesis: A Bioinformatic Circuit Diagram of the Hematopoietic Differentiation Control.

MicroRNAs (miRs) are a recently realized class of epigenetic elements which block translation of mRNA to protein, and which also may result in gene silencing through chromatin remodeling. MicroRNAs have been shown to control cellular metabolism, differentiation and development in numerous organisms including drosophila, rat, mouse, and humans. Recently, miRs have been implicated in the control of hematopoiesis. Even more importantly, both aberrant expression and deletion are highly associated with the development of various cancers. To better understand the role of miRs in normal hematopoiesis, the inventors have determined the microRNA expression profile of primary normal human PBSC and bone marrow CD34+ hematopoietic stem-progenitor cells (HSPC). The inventors have combined this data with the extensive mRNA expression data obtained from CD34+ HSPC, CD34+/CD38−ILin−HSC-enriched, and CD34+/CD38+/Lin+HPC-enriched populations in a previous study. (Cancer Research 64:4344) Combining these two datasets into one integrated database has allowed the inventors to intricately examine the global interaction of HSPC mRNAs and microRNAs, and to also predict which miRs are involved with differentiation of the hematopoietic system through their control of expression of hematopoieticly important proteins. The inventors have confirmed that expression of a number of these hematopoieticly important proteins are in fact controlled by microRNAs. Based on the bioinformatic and protein expression studies, the inventors present a global model by which microRNAs control hematopoietic differentiation. These findings offer promising new targets for the study of stem cell differentiation and targets for genetic therapies of hematopoietic stem cells.

MicroRNA hsa-mir-155 Blocks Myeloid and Erythroid Differentiation of Human CD34+ HSPC.

In a previous study, the inventors determined all of the microRNAs expressed by human CD34+ hematopoietic stem progenitor cells from bone marrow and GM-CSF mobilized periferal blood. When the inventors combined microRNA expression with mRNA expression data from a previous study (Georgantas et al, Cancer Research 64:4434), the inventors were able to bioinformatically predict the actions of microRNAs within the hematopoietic system. MicroRNA hsa-mir-155 was highly expressed in CD34+ HSPC, and was immediately identified to be a possible generalized inhibitor of hematopoietic differentiation. MicroRNA-155 was predicted to interact with HSPC expressed mRNAs proteins previously shown to be highly involved myeloid and erythroid differentiation. K562 cells were transduced with hsa-mir-155 lentivirus, and were then differentiated with TPA to megakaryocytes or with hemin to erythrocytes. Compared to controls mir-155 reduced megakaryocyte differentiation by approximately 70%, and erythropoiesis by over 90%. Cell growth was not effected by mir-155, indicating this was a block in differentiation, and not simply a block in cell growth. Transduction of human PBSC CD34+ cells, followed by colony forming assay, revealed that mir-155 reduces both myeloid and erythroid colony formation by greater than 70%. The inventors are currently further testing the effect of mir-155 on PBSC in suspension cultures and transplant into NOG mice. These data strongly indicate that mir-155 acts in control of myeloid and erythroid differentiation.

MicroRNA hsa-mir-16 Contributes to Control of Myeloid Differentiation of Human CD34+ HSPC.

In a previous study, the inventors determined all of the microRNAs expressed by human CD34+ hematopoietic stem-progenitor cells from bone marrow and GM-CSF mobilized peripheral blood. When the inventors combined microRNA expression with mRNA expression data from a previous study (Georgantas et al, Cancer Research 64:4434), the inventors were able to bio-informaticly predict the actions of microRNAs within the hematopoietic system. MicroRNA hsa-mir-16 was highly expressed in CD34+ HSPC, and based on it predicted target mRNAs was identified as a possible regulator of myeloid differentiation. The inventors first confirmed that protein expression from these putative target mRNAs were in fact regulated by mir-16. The 3′UTR sequences from these mRNAs were cloned behind a luciferase reporter construct, which were then transfected into K562 cells, which highly express mir-16. In all cases protein expression was reduced.

### REFERENCES

1. Finnegan, E J & Matzke, M A. (2003) J Cell Sci 116, 4689-4693.[Free Full Text]
2. Bartel, D P. (2004) Cell 116, 281-297.[CrossRef][ISI][Medline]
3. Chen, C Z, Li, L, Lodish, H F & Bartel, D P. (2004) Science 303, 83-86.[Abstract/Free Full Text]
4. Felli, N, Fontana, L, Pelosi, E, Botta, R, Bonci, D, Facchiano, F, Liuzzi, F, Lulli, V, Morsilli, O & Santoro, S, et al. (2005) Proc Natl Acad Sci USA 102, 18081-18086.[Abstract/Free Full Text]
5. Fazi, F, Rosa, A, Fatica, A, Gelmetti, V, De Marchis, M L, Nervi, C & Bozzoni, I. (2005) Cell 123, 819-831.[CrossRef] [SI][Medline]
6. Calin, G A, Sevignani, C, Dumitru, C D, Hyslop, T, Noch, E, Yendamuri, S, Shimizu, M, Rattan, S, Bullrich, F & Negrini, M, et al. (2004) Proc Natl Acad Sci USA 101, 2999-3004.[Abstract/Free Full Text]
7. Calin, G A, Dumitru, C D, Shimizu, M, Bichi, R, Zupo, S, Noch, E, Aldler, H, Rattan, S, Keating, M & Rai, K, et al. (2002) Proc Natl Acad Sci USA 99, 15524-15529.[Abstract/Free Full Text]
8. Georgantas, R W III, Tanadve, V, Malehorn, M, Heimfeld, S, Chen, C, Carr, L, Martinez-Murillo, F, Riggins, G, Kowalski, J & Civin, C I. (2004) Cancer Res 64, 4434-4441.[Abstract/Free Full Text]
9. John, B, Enright, A J, Aravin, A, Tuschl, T, Sander, C & Marks, DS. (2004) PLoS Biol 2, e363.[CrossRef][Medline]
10. Lewis, B P, Burge, C B & Bartel, D P. (2005) Cell 120, 15-20.[CrossRef][ISI][Medline]
11. Lewis, B P, Shih, I H, Jones-Rhoades, M W, Bartel, D P & Burge, C B. (2003) Cell 115, 787-798.[CrossRef][ISI][Medline]
12. Bentwich, I. (2005) FEBS Lett 579, 5904-5910.[CrossRef][ISI][Medline]
13. Elagib, K E, Xiao, M, Hussaini, I M, Delehanty, L L, Palmer, L A, Racke, F K, Birrer, M J, Shanmugasundaram, G, McDevitt, M A & Goldfarb, A N. (2004) Mol Cell Biol 24, 7779-7794.[Abstract/Free Full Text]
14. Goldfarb, A N, Delehanty, L L, Wang, D, Racke, F K & Hussaini, I M. (2001) J Biol Chem 276, 29526-29530.[Abstract/Free Full Text]
15. Xie, P, Chan, F S, Ip, N Y & Leung, M F. (1999) Leuk Res 23, 1113-1119.[CrossRef]
16. Unwin, R D, Smith, D L, Blinco, D, Wilson, C L, Miller, C J, Evans, C A, Jaworska, E, Baldwin, S A, Barnes, K & Pierce, A, et al. (2006) Blood 107, 4687-4694.[Abstract/Free Full Text]
17. Kondo, M, Wagers, A J, Manz, M G, Prohaska, S S, Scherer, D C, Beilhack, G F, Shizuru, J A & Weissman, I L. (2003) Annu Rev Immunol 21, 759-806.[CrossRef][ISI][Medline]
18. Sauvageau, G, Iscove, N N & Humphries, R K. (2004) Oncogene 23, 7223-7232.[CrossRef][ISI][Medline]
19. Lessard, J & Sauvageau, G. (2003) Nature 423, 255-260.[CrossRef][Medline]
20. Park, I K, Qian, D, Kiel, M, Becker, M W, Pihalja, M, Weissman, I L, Morrison, S J & Clarke, M F. (2003) Nature 423, 302-305.[CrossRef][Medline]
21. Blobel, G A. (2000) Blood 95, 745-755.[Free Full Text]
22. Kasper, L H, Boussouar, F, Ney, P A, Jackson, C W, Rehg, J, van Deursen, J M & Brindle, P K. (2002) Nature 419, 738-743.[CrossRef][Medline]
23. Nagasawa, E, Abe, Y, Nishimura, J, Yanase, T, Nawata, H & Muta, K. (2005) Exp Hematol 857-864.[CrossRef] [SI][Medline]
24. Zhang, W & Bieker, J J. (1998) Proc Natl Acad Sci USA 95, 9855-9860.[Abstract/Free Full Text]
25. Azcoitia, V, Aracil, M, Martinez, A C & Torres, M. (2005) Dev Biol 280, 307-320.[CrossRef][ISI][Medline]
26. Hisa, T, Spence, S E, Rachel, R A, Fujita, M, Nakamura, T, Ward, J M, Devor-Henneman, D E, Saiki, Y, Kutsuna, H & Tessarollo, L, et al. (2004) EMBO J 23, 450-459.[CrossRef][ISI][Medline]
27. Matsumura, T, Kametani, Y, Ando, K, Hirano, Y, Katano, I, Ito, R, Shiina, M, Tsukamoto, H, Saito, Y & Tokuda, Y, et al. (2003) Exp Hematol 31, 789-797.[CrossRef][ISI][Medline]
28. Ramkissoon, S H, Mainwaring, L A, Ogasawara, Y, Keyvanfar, K, McCoy, J P Jr, Sloand, E M, Kajigaya, S & Young, N S. (2006) Leuk Res 30, 643-647.[CrossRef][ISI][Medline]
29. Racke, F K, Wang, D, Zaidi, Z, Kelley, J, Visvader, J, Soh, J W & Goldfarb, A N. (2001) J Biol Chem 276, 522-528.[Abstract/Free Full Text]
30. Liu, C-G, Calin, G A, Meloon, B, Gamliel, N, Sevignani, C, Ferracin, M, Dumitru, C D, Shimizu, M, Zupo, S & Dono, M, et al. (2004) Proc Natl Acad Sci USA 101, 9740-9744.[Abstract/Free Full Text]
31. Yu, X, Zhan, X, D'Costa, J, Tanavde, V M, Ye, Z, Peng, T, Malehorn, M T, Yang, X, Civin, C I & Cheng, L. (2003) Mol Ther 7, 827-838.[CrossRef] [ISI][Medline]
32. Bartel, D P. (2004) Cell 116, 281-297.[CrossRef][ISI][Medline]
33. Zambidis, E T, Peault, B, Park, T S, Bunz, F & Civin, C I. (2005) Blood 106, 860-870.[Abstract/Free Full Text]
34. Yu, X, Alder, J K, Chun, J H, Friedman, A D, Heimfeld, S, Cheng, L & Civin, C I. (2006) Stem Cells 24, 876-888.[Abstract/Free Full Text]

### Example 3

TABLE 3
miRNA to mRNA mappings. Predicted mRNA targets (expressed in PBSC and
BM) for each individual miRNA (noted above sub-tables) as identified by the
Transcriptome Interaction Database. Included are the Ensemble Gene ID, the HUGO
systematic gene symbol, the Unigene identifier, the normalized expression level of the
mRNA [in relative fluorescence units (RFU)], and the Affymetrix call flags for each
transcript (P, present/expressed).
92

| Ensemble gene | Gene | | PBSC, | PBSC | BM, | BM |
|---|---|---|---|---|---|---|
| ID | symbol | Unigene | RFU | flags | RFU | flags |
| ENSG00000137845 | ADAM10 | Hs.172028 | 221.1 | PP | 315.55 | PP |
| ENSG00000138031 | ADCY3 | Hs.467898 | 208.6 | PP | 178.4 | PP |
| ENSG00000087274 | ADD1 | Hs.183706 | 410.2 | PP | 214.3 | PP |
| ENSG00000143401 | ANP32E | Hs.385913 | 169.2 | PP | 423.3 | PP |
| ENSG00000143761 | ARF1 | Hs.286221 | 1245.2 | PP | 798.4 | PP |
| ENSG00000163466 | ARPC2 | Hs.529303 | 1447.8 | PP | 1774.7 | PP |
| ENSG00000143153 | ATP1B1 | Hs.291196 | 132.4 | PP | 245.55 | PP |
| ENSG00000174437 | ATP2A2 | Hs.506759 | 203.5 | PP | 255.4 | PP |
| ENSG00000155096 | AZIN1 | Hs.459106 | 551.2 | PP | 619.2 | PP |
| ENSG00000119861 | BCL11A | Hs.370549 | 363.1 | PP | 387.8 | PP |
| ENSG00000159388 | BTG2 | Hs.519162 | 588.2 | PP | 538.3 | PP |
| ENSG00000171067 | C11orf24 | Hs.303025 | 127.9 | PP | 105.45 | PA |
| ENSG00000139746 | C13orf10 | Hs.131715 | 119.8 | PP | 178.7 | PP |
| ENSG00000152242 | C18orf25 | Hs.116486 | 129.7 | PP | 130.6 | PA |
| ENSG00000117616 | C1orf63 | Hs.259412 | 1202.1 | PP | 1000.85 | PP |
| ENSG00000132823 | C20orf111 | Hs.75798 | 346 | PP | 259.1 | PP |
| ENSG00000031003 | C5orf5 | Hs.483473 | 256.3 | PP | 316.85 | PP |
| ENSG00000132424 | C6orf111 | Hs.520287 | 674.8 | PP | 743.75 | PP |
| ENSG00000112308 | C6orf62 | Hs.519930 | 330.6 | PP | 509.45 | PP |
| ENSG00000067955 | CBFB | Hs.460988 | 719.1 | PP | 1032 | PP |
| ENSG00000112237 | CCNC | Hs.430646 | 273.3 | PP | 380.8 | PP |
| ENSG00000110848 | CD69 | Hs.208854 | 1631.5 | PP | 1082.1 | PP |
| ENSG00000070831 | CDC42 | Hs.467637 | 620.1 | PP | 437.75 | PP |
| ENSG00000172216 | CEBPB | Hs.517106 | 374.1 | PP | 391 | PP |
| ENSG00000166200 | COPS2 | Hs.369614 | 521.2 | PP | 700.65 | PP |
| ENSG00000102974 | CTCF | Hs.368367 | 506.1 | PP | 535.55 | PA |
| ENSG00000036257 | CUL3 | Hs.372286 | 554.5 | PP | 473.25 | PP |
| ENSG00000168209 | DDIT4 | Hs.523012 | 736.8 | PP | 535.85 | PP |
| ENSG00000124487 | DDX3X | Hs.380774 | 769.8 | PP | 1229 | PP |
| ENSG00000067048 | DDX3Y | Hs.99120 | 302.6 | PP | 309.6 | PA |
| ENSG00000132153 | DHX30 | Hs.517948 | 209.5 | PP | 193.5 | PP |
| ENSG00000082458 | DLG3 | Hs.522680 | 136.2 | PP | 89.45 | PP |
| ENSG00000128590 | DNAJB9 | Hs.6790 | 211.5 | PP | 433.55 | PP |
| ENSG00000167264 | DUS2L | Hs.534460 | 140.7 | PP | 111.5 | PA |
| ENSG00000143507 | DUSP10 | Hs.497822 | 328.9 | PP | 371.8 | PP |
| ENSG00000139318 | DUSP6 | Hs.298654 | 166.6 | PP | 316.85 | PP |
| ENSG00000112242 | E2F3 | Hs.269408 | 295.6 | PP | 585.95 | PP |
| ENSG00000147654 | EBAG9 | Hs.409368 | 293.7 | PP | 285.65 | PP |
| ENSG00000123908 | EIF2C2 | Hs.449415 | 150.2 | PP | 115.85 | PP |
| ENSG00000104131 | EIF3S1 | Hs.404056 | 340.4 | PP | 365.05 | PP |
| ENSG00000110321 | EIF4G2 | Hs.183684 | 2137 | PP | 2701.9 | PP |
| ENSG00000106462 | EZH2 | Hs.444082 | 162.9 | PP | 216.25 | PP |
| ENSG00000166147 | FBN1 | Hs.146447 | 195.2 | PP | 178.7 | PP |
| ENSG00000143756 | FBXO28 | Hs.64691 | 106.1 | PP | 70.5 | PA |
| ENSG00000151702 | FLI1 | Hs.504281 | 156.4 | PP | 248.05 | PP |
| ENSG00000168137 | FLJ10707 | Hs.288164 | 452.5 | PP | 256.1 | PM |
| ENSG00000159202 | FLJ13855 | Hs.514297 | 539 | PP | 525.1 | PP |
| ENSG00000109920 | FNBP4 | Hs.6834 | 501.2 | PP | 497.2 | PP |
| ENSG00000163430 | FSTL1 | Hs.269512 | 408.6 | PP | 227.95 | PP |
| ENSG00000114416 | FXR1 | Hs.478407 | 143.9 | PP | 155.75 | PP |
| ENSG00000138757 | G3BP2 | Hs.303676 | 242.8 | PP | 428.55 | PP |
| ENSG00000196700 | GM632 | Hs.551552 | 103.5 | PP | 145.05 | PP |
| ENSG00000175265 | GM88 | Hs.182982 | 627 | PP | 822.4 | PP |
| ENSG00000147533 | GOLGA7 | Hs.7953 | 669.8 | PP | 592.25 | PP |
| ENSG00000159592 | GPBP1L1 | Hs.238432 | 275.7 | PP | 300.05 | PP |
| ENSG00000128731 | HERC2 | Hs.434890 | 257.7 | PP | 239.3 | PP |
| ENSG00000163349 | HIPK1 | Hs.532363 | 347.7 | PP | 488.5 | PP |
| ENSG00000122566 | HNRPA2B1 | Hs.487774 | 1802.2 | PP | 2268.3 | PP |
| ENSG00000170144 | HNRPA3 | Hs.516539 | 375.5 | PP | 558.6 | PP |
| ENSG00000169045 | HNRPH1 | Hs.202166 | 1986.4 | PP | 2132.6 | PP |
| ENSG00000153807 | HOXA10 | Hs.110637 | 161.3 | PP | 200.7 | PA |
| ENSG00000078399 | HOXA9 | Hs.127428 | 739.2 | PP | 674 | PP |
| ENSG00000164164 | HSHIN1 | Hs.270851 | 303.1 | PP | 312.65 | PP |
| ENSG00000144381 | HSPD1 | Hs.113684 | 2868.6 | PP | 5689.3 | PP |
| ENSG00000168556 | ING2 | Hs.107153 | 177 | PP | 151.85 | PP |
| ENSG00000186480 | INSIG1 | Hs.520819 | 196.9 | PP | 304.7 | PP |
| ENSG00000145703 | IQGAP2 | Hs.291030 | 537.8 | PP | 457.9 | PP |
| ENSG00000143164 | IQWD1 | Hs.435741 | 168.5 | PP | 156.4 | PP |
| ENSG00000150093 | ITGB1 | Hs.429052 | 865.6 | PP | 593.85 | PP |
| ENSG00000177606 | JUN | Hs.525704 | 1300.5 | PP | 2286.95 | PP |
| ENSG00000179889 | KIAA0251 | Hs.370781 | 386 | PP | 331 | PM |
| ENSG00000126775 | KIAA0831 | Hs.414809 | 254.4 | PP | 217.45 | PP |
| ENSG00000170759 | KIF5B | Hs.327736 | 346.6 | PP | 430.75 | PP |
| ENSG00000127528 | KLF2 | Hs.107740 | 609 | PP | 859.3 | PP |
| ENSG00000136826 | KLF4 | Hs.376206 | 536.5 | PP | 1193.7 | PP |
| ENSG00000166783 | LKAP | Hs.173524 | 135 | PP | 182.75 | PP |
| ENSG00000152102 | LOC130074 | Hs.469941 | 508.5 | PP | 344.1 | PP |
| ENSG00000127603 | MACF1 | Hs.472475 | 222.8 | PP | 248.35 | PP |
| ENSG00000121579 | MAK3 | Hs.269528 | 268.8 | PP | 307 | PP |
| ENSG00000012983 | MAP4K5 | Hs.130491 | 66 | PP | 116 | PP |
| ENSG00000081189 | MEF2C | Hs.444409 | 426 | PP | 437.85 | PP |
| ENSG00000165943 | MOAP1 | Hs.24719 | 527.1 | PP | 299.1 | PP |
| ENSG00000159256 | MORC3 | Hs.421150 | 108.8 | PP | 130.45 | PP |
| ENSG00000147649 | MTDH | Hs.377155 | 258.6 | PP | 329.8 | PP |
| ENSG00000005810 | MYCBP2 | Hs.151411 | 187 | PP | 643.55 | PP |
| ENSG00000137420 | NOL7 | Hs.549161 | 1095.5 | PP | 1222.05 | PP |
| ENSG00000141458 | NPC1 | Hs.464779 | 126.1 | PP | 135.8 | PM |
| ENSG00000106459 | NRF1 | Hs.298069 | 157.5 | PP | 126.8 | PA |
| ENSG00000180530 | NRIP1 | Hs.155017 | 842 | PP | 914.85 | PP |
| ENSG00000035681 | NSMAF | Hs.372000 | 517.1 | PP | 517.75 | PP |
| ENSG00000147162 | OGT | Hs.405410 | 165.1 | PP | 183.6 | PP |
| ENSG00000172939 | OXSR1 | Hs.475970 | 211 | PP | 186.15 | PA |
| ENSG00000172239 | PAIP1 | Hs.482038 | 127.8 | PP | 157 | PP |
| ENSG00000090060 | PAPOLA | Hs.253726 | 307.4 | PP | 286 | PP |
| ENSG00000185619 | PCGF3 | Hs.144309 | 192.1 | PP | 237.55 | PP |
| ENSG00000102225 | PCTK1 | Hs.496068 | 135 | PP | 88.45 | PA |
| ENSG00000059758 | PCTK2 | Hs.506415 | 118.3 | PP | 220.9 | PP |
| ENSG00000132326 | PER2 | Hs.58756 | 162.6 | PP | 308.65 | PP |
| ENSG00000112511 | PHF1 | Hs.166204 | 903.7 | PP | 604.1 | PP |
| ENSG00000043143 | PHF15 | Hs.483419 | 234 | PP | 320.3 | PP |
| ENSG00000118482 | PHF3 | Hs.348921 | 260 | PP | 268.9 | PP |
| ENSG00000051382 | PIK3CB | Hs.239818 | 275.1 | PP | 280.95 | PP |
| ENSG00000154217 | PITPNC1 | Hs.549130 | 391.3 | PP | 316.6 | PP |
| ENSG00000112941 | POLS | Hs.481542 | 355.5 | PP | 367.35 | PP |
| ENSG00000127125 | PPCS | Hs.549196 | 120.7 | PP | 188.55 | PP |
| ENSG00000058272 | PPP1R12A | Hs.49582 | 154.9 | PP | 178.75 | PP |
| ENSG00000005249 | PRKAR2B | Hs.433068 | 200.7 | PP | 237.75 | PP |
| ENSG00000138430 | PTD004 | Hs.157351 | 848.2 | PP | 1052.6 | PP |
| ENSG00000171522 | PTGER4 | Hs.199248 | 531.8 | PP | 514.8 | PP |
| ENSG00000112531 | QKI | Hs.510324 | 284.1 | PP | 564.45 | PP |
| ENSG00000119396 | RAB14 | Hs.371563 | 140.5 | PP | 198.15 | PP |
| ENSG00000152061 | RABGAP1L | Hs.495391 | 229.4 | PP | 396.75 | PP |
| ENSG00000164754 | RAD21 | Hs.81848 | 281.3 | PP | 414.9 | PP |
| ENSG00000010017 | RANBP9 | Hs.306242 | 381.3 | PP | 489.95 | PP |
| ENSG00000116473 | RAP1A | Hs.190334 | 217.2 | PP | 367.75 | PP |
| ENSG00000127314 | RAP1B | Hs.369920 | 1093.1 | PP | 1537.75 | PP |
| ENSG00000137075 | RNF38 | Hs.333503 | 322.6 | PP | 227.7 | PP |
| ENSG00000063978 | RNF4 | Hs.66394 | 115.8 | PP | 325.65 | PP |
| ENSG00000198838 | RYR3 | Hs.369250 | 116.8 | PP | 131.8 | PP |
| ENSG00000182568 | SATB1 | Hs.517717 | 829.9 | PP | 652.05 | PP |
| ENSG00000121892 | SCC-112 | Hs.331431 | 220.4 | PP | 258.2 | PP |
| ENSG00000156642 | SDFR1 | Hs.187866 | 169.4 | PP | 193.05 | PP |
| ENSG00000179833 | SERTAD2 | Hs.77293 | 498.4 | PP | 556.2 | PP |
| ENSG00000096717 | SIRT1 | Hs.369779 | 133.8 | PP | 124.9 | PP |
| ENSG00000134294 | SLC38A2 | Hs.221847 | 634.3 | PP | 1026.55 | PP |
| ENSG00000184602 | SNN | Hs.459952 | 234.4 | PP | 239.3 | PP |
| ENSG00000137642 | SORL1 | Hs.368592 | 2264.1 | PP | 1084.65 | PP |
| ENSG00000196700 | SOX18 | Hs.8619 | 1264.6 | PP | 2674 | PP |
| ENSG00000124766 | SOX4 | Hs.357901 | 2351.7 | PP | 2500.65 | PP |
| ENSG00000135250 | SRPK2 | Hs.285197 | 106.2 | PP | 118.7 | PM |
| ENSG00000101972 | STAG2 | Hs.496710 | 392 | PP | 375.2 | PP |
| ENSG00000198648 | STK39 | Hs.276271 | 129.8 | PP | 111.35 | PP |
| ENSG00000173812 | SUI1 | Hs.150580 | 6046 | PP | 6098.85 | PP |
| ENSG00000165169 | TCTE1L | Hs.446392 | 323 | PP | 369.75 | PP |
| ENSG00000114126 | TFDP2 | Hs.379018 | 253.2 | PP | 196.3 | PA |
| ENSG00000177426 | TGIF | Hs.373550 | 116.7 | PP | 95.35 | PM |
| ENSG00000183864 | TOB2 | Hs.474978 | 165.3 | PP | 196.4 | PP |
| ENSG00000198900 | TOP1 | Hs.472737 | 315.5 | PP | 468.35 | PP |
| ENSG00000165699 | TSC1 | Hs.370854 | 232.7 | PP | 242.3 | PP |
| ENSG00000132388 | UBE2G1 | Hs.462035 | 272.5 | PP | 373.45 | PP |
| ENSG00000169764 | UGP2 | Hs.516217 | 656.9 | PP | 560.95 | PP |
| ENSG00000164828 | UNC84A | Hs.438072 | 146.4 | PP | 111.15 | PP |
| ENSG00000135655 | USP15 | Hs.434951 | 197.3 | PP | 160.35 | PP |
| ENSG00000143952 | VPS54 | Hs.48499 | 187.9 | PP | 151.5 | PP |
| ENSG00000198373 | WWP2 | Hs.408458 | 231 | PP | 185.05 | PP |
| ENSG00000100219 | XBP1 | Hs.437638 | 1126.5 | PP | 826.5 | PP |
| ENSG00000100811 | YY1 | Hs.388927 | 539.7 | PP | 740.2 | PP |
| ENSG00000170949 | ZNF160 | Hs.467236 | 1056.4 | PP | 1016.85 | PP |
| ENSG00000179456 | ZNF238 | Hs.69997 | 235.7 | PP | 241.8 | PP |
| Ensemble Gene | | | PBSC | PBSC | | BM |
| ID | Gene Symbol | Unigene | RFU | Flags | BM RFU | Flags |

223

| | | | | | | |
|---|---|---|---|---|---|---|
| ENSG00000004455 | AK2 | Hs.470907 | 303.6 | PP | 498.6 | PP |
| ENSG00000117020 | AKT3 | Hs.498292 | 246.4 | PP | 259.35 | PP |
| ENSG00000170017 | ALCAM | Hs.150693 | 528.9 | PP | 695.45 | PP |
| ENSG00000101844 | APG4A | Hs.8763 | 139.4 | PP | 172.75 | PA |
| ENSG00000117713 | ARID1A | Hs.468972 | 477.6 | PP | 444.8 | PP |
| ENSG00000143153 | ATP1B1 | Hs.291196 | 132.4 | PP | 245.55 | PP |
| ENSG00000126749 | C2F | Hs.504628 | 383.4 | PP | 460.7 | PM |
| ENSG00000168958 | C2orf33 | Hs.471528 | 235.3 | PP | 302.3 | PP |
| ENSG00000136925 | C9orf97 | Hs.494595 | 207.5 | PP | 180.4 | PP |
| ENSG00000135932 | CAB39 | Hs.471593 | 527.9 | PP | 637.3 | PP |
| ENSG00000142453 | CARM1 | Hs.371416 | 141.6 | PP | 105.7 | PA |
| ENSG00000067955 | CBFB | Hs.460988 | 719.1 | PP | 1032 | PP |
| ENSG00000108468 | CBX1 | Hs.77254 | 432 | PP | 415.85 | PP |
| ENSG00000082258 | CCNT2 | Hs.292754 | 114.4 | PP | 102.75 | PP |
| ENSG00000172216 | CEBPB | Hs.517106 | 374.1 | PP | 391 | PP |
| ENSG00000153922 | CHD1 | Hs.519474 | 89.4 | PP | 202.95 | PP |
| ENSG00000072609 | CHFR | Hs.507336 | 247.4 | PP | 253.05 | PP |
| ENSG00000166200 | COPS2 | Hs.369614 | 521.2 | PP | 700.65 | PP |
| ENSG00000108848 | CROP | Hs.130293 | 2166.4 | PP | 1637.55 | PP |
| ENSG00000048740 | CUGBP2 | Hs.309288 | 159.6 | PP | 334.5 | PP |
| ENSG00000160967 | CUTL1 | Hs.438974 | 333.3 | PP | 412.55 | PP |
| ENSG00000121966 | CXCR4 | Hs.421986 | 532.4 | PP | 1919.4 | PP |
| ENSG00000166347 | CYB5 | Hs.465413 | 191.8 | PP | 277.55 | PP |
| ENSG00000168209 | DDIT4 | Hs.523012 | 736.8 | PP | 535.85 | PP |
| ENSG00000124487 | DDX3X | Hs.380774 | 769.8 | PP | 1229 | PP |
| ENSG00000136986 | DERL1 | Hs.241576 | 223 | PP | 240.65 | PA |
| ENSG00000109606 | DHX15 | Hs.5683 | 1219.8 | PP | 1620.75 | PP |
| ENSG00000112242 | E2F3 | Hs.269408 | 295.6 | PP | 585.95 | PP |
| ENSG00000186676 | EEF1G | Hs.144835 | 19027.3 | PP | 14878.45 | PP |
| ENSG00000110321 | EIF4G2 | Hs.183684 | 2137 | PP | 2701.9 | PP |
| ENSG00000004478 | FKBP4 | Hs.524183 | 490.7 | PP | 372.15 | PM |
| ENSG00000118689 | FOXO3A | Hs.220950 | 1368.3 | PP | 720.35 | PP |
| ENSG00000104064 | GABPB2 | Hs.546852 | 129.3 | PP | 183.75 | PM |
| ENSG00000116717 | GADD45A | Hs.80409 | 266 | PP | 245.7 | PP |
| ENSG00000141429 | GALNT1 | Hs.514806 | 220 | PP | 273.8 | PP |
| ENSG00000106070 | GRB10 | Hs.164060 | 550.7 | PP | 574.15 | PP |
| ENSG00000143321 | HDGF | Hs.506748 | 243.6 | PP | 858.8 | PP |
| ENSG00000013583 | HEBP1 | Hs.294133 | 387.7 | PP | 516.4 | PP |
| ENSG00000114315 | HES1 | Hs.250666 | 232.7 | PP | 169.8 | PP |
| ENSG00000152804 | HHEX | Hs.118651 | 328.9 | PP | 298.65 | PP |
| ENSG00000078140 | HIP2 | Hs.50308 | 60.5 | PP | 68.4 | PP |
| ENSG00000010818 | HIVEP2 | Hs.510172 | 149.6 | PP | 157.9 | PP |
| ENSG00000153187 | HNRPU | Hs.166463 | 470.3 | PP | 887.6 | PP |
| ENSG00000078399 | HOXA9 | Hs.127428 | 739.2 | PP | 674 | PP |
| ENSG00000164164 | HSHIN1 | Hs.270851 | 303.1 | PP | 312.65 | PP |
| ENSG00000102753 | KPNA3 | Hs.527919 | 250.8 | PP | 391.35 | PP |
| ENSG00000133703 | KRAS | Hs.505033 | 355.8 | PP | 363.05 | PP |
| ENSG00000143815 | LBR | Hs.435166 | 1166 | PP | 1807.35 | PP |
| ENSG00000135363 | LMO2 | Hs.34560 | 1997.3 | PP | 1809.95 | PP |
| ENSG00000110031 | LPXN | Hs.125474 | 233.9 | PP | 209.7 | PP |
| ENSG00000171492 | LRRC8D | Hs.482087 | 543.3 | PP | 374.45 | PP |
| ENSG00000127603 | MACF1 | Hs.472475 | 222.8 | PP | 248.35 | PP |
| ENSG00000121579 | MAK3 | Hs.269528 | 268.8 | PP | 307 | PP |
| ENSG00000047849 | MAP4 | Hs.517949 | 302.6 | PP | 276.5 | PP |
| ENSG00000180398 | MCFD2 | Hs.293689 | 289.4 | PP | 258.25 | PP |
| ENSG00000081189 | MEF2C | Hs.444409 | 426 | PP | 437.85 | PP |
| ENSG00000133026 | MYH10 | Hs.16355 | 375.1 | PP | 362.5 | PP |
| ENSG00000164190 | NIPBL | Hs.481927 | 165.1 | PP | 145.15 | PP |
| ENSG00000106459 | NRF1 | Hs.298069 | 157.5 | PP | 126.8 | PA |
| ENSG00000162231 | NXF1 | Hs.523739 | 543 | PP | 626 | PP |
| ENSG00000059758 | PCTK2 | Hs.506415 | 118.3 | PP | 220.9 | PP |
| ENSG00000025293 | PHF20 | Hs.517044 | 249.8 | PP | 173.65 | PP |
| ENSG00000073921 | PICALM | Hs.163893 | 357.3 | PP | 315.6 | PP |
| ENSG00000104695 | PPP2CB | Hs.491440 | 197.6 | PP | 323.9 | PP |
| ENSG00000117569 | PTBP2 | Hs.269895 | 183 | PP | 160.15 | PP |
| ENSG00000138430 | PTD004 | Hs.157351 | 848.2 | PP | 1052.6 | PP |
| ENSG00000171862 | PTEN | Hs.500466 | 353.7 | PP | 334.4 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000103769 | RAB11A | Hs.321541 | 1087.7 | PP | 1020.15 | PP |
| ENSG00000138069 | RAB1A | Hs.310645 | 70.8 | PP | 109.75 | PM |
| ENSG00000152061 | RABGAP1L | Hs.495391 | 229.4 | PP | 396.75 | PP |
| ENSG00000137040 | RANBP6 | Hs.167496 | 125.2 | PP | 140.05 | PP |
| ENSG00000127314 | RAP1B | Hs.369920 | 1093.1 | PP | 1537.75 | PP |
| ENSG00000145715 | RASA1 | Hs.553501 | 138.8 | PP | 103.6 | PP |
| ENSG00000197676 | RBM16 | Hs.227602 | 193.8 | PP | 274 | PP |
| ENSG00000147274 | RBMX | Hs.380118 | 1610.3 | PP | 1490.7 | PP |
| ENSG00000168214 | RBPSUH | Hs.479396 | 79.3 | PP | 100.35 | PP |
| ENSG00000117906 | RCN2 | Hs.79088 | 121.2 | PP | 121.5 | PP |
| ENSG00000157916 | RER1 | Hs.525527 | 262.6 | PP | 329.9 | PP |
| ENSG00000170633 | RNF34 | Hs.292804 | 424.6 | PP | 385.65 | PP |
| ENSG00000063978 | RNF4 | Hs.66394 | 115.8 | PP | 325.65 | PP |
| ENSG00000155878 | SEC22L1 | Hs.534212 | 662.9 | PP | 588.95 | PP |
| ENSG00000125354 | 6-Sep | Hs.496666 | 335.7 | PP | 310.55 | PP |
| ENSG00000153914 | SFRS12 | Hs.519347 | 258 | PP | 229.6 | PP |
| ENSG00000161547 | SFRS2 | Hs.73965 | 3723.5 | PP | 4390.4 | PP |
| ENSG00000124523 | SIRT5 | Hs.282331 | 252.8 | PP | 171.5 | PM |
| ENSG00000138821 | SLC39A8 | Hs.288034 | 290.7 | PP | 275.05 | PP |
| ENSG00000159140 | SON | Hs.517262 | 408.8 | PP | 467.15 | PP |
| ENSG00000172845 | SP3 | Hs.531587 | 137.8 | PP | 293.35 | PP |
| ENSG00000065526 | SPEN | Hs.270499 | 257.6 | PP | 262.7 | PP |
| ENSG00000115306 | SPTBN1 | Hs.503178 | 589.9 | PP | 344.65 | PP |
| ENSG00000149136 | SSRP1 | Hs.523680 | 329.8 | PP | 503.65 | PP |
| ENSG00000167323 | STIM1 | Hs.501735 | 143.2 | PP | 99.7 | PP |
| ENSG00000198648 | STK39 | Hs.276271 | 129.8 | PP | 111.35 | PP |
| ENSG00000117632 | STMN1 | Hs.209983 | 546.6 | PP | 663.4 | PP |
| ENSG00000135316 | SYNCRIP | Hs.472056 | 178 | PP | 315.75 | PP |
| ENSG00000136560 | TANK | Hs.516490 | 307.6 | PP | 221.95 | PP |
| ENSG00000113649 | TCERG1 | Hs.443465 | 196.5 | PP | 257.25 | PP |
| ENSG00000140262 | TCF12 | Hs.511504 | 179.4 | PP | 439.15 | PP |
| ENSG00000139644 | TEGT | Hs.35052 | 757.7 | PP | 1052.5 | PP |
| ENSG00000106829 | TLE4 | Hs.444213 | 309.2 | PP | 251.15 | PP |
| ENSG00000062716 | TMEM49 /// | Hs.444569 | 425.2 | PP | 441.3 | PP |
| | MIRN21 | | | | | |
| ENSG00000173726 | TOMM20 | Hs.533192 | 656.5 | PP | 888.45 | PP |
| ENSG00000166598 | TRA1 | Hs.192374 | 151 | PP | 339.35 | PP |
| ENSG00000077721 | UBE2A | Hs.379466 | 299.1 | PP | 400.85 | PP |
| ENSG00000170832 | USP32 | Hs.132868 | 120.2 | PP | 118.35 | PP |
| ENSG00000146457 | WTAP | Hs.446091 | 400.8 | PP | 406.6 | PP |
| ENSG00000179456 | ZNF238 | Hs.69997 | 235.7 | PP | 241.8 | PP |
| ENSG00000112200 | ZNF451 | Hs.485628 | 175.7 | PP | 176.15 | PP |

146

| | | | | | | |
|---|---|---|---|---|---|---|
| ENSG00000114770 | ABCC5 | Hs.368563 | 373.4 | PP | 322.1 | PM |
| ENSG00000115091 | ACTR3 | Hs.433512 | 353.8 | PP | 673.3 | PP |
| ENSG00000143761 | ARF1 | Hs.286221 | 1245.2 | PP | 798.4 | PP |
| ENSG00000143153 | ATP1B1 | Hs.291196 | 132.4 | PP | 245.55 | PP |
| ENSG00000175334 | BANF1 | Hs.433759 | 387.5 | PP | 385 | PP |
| ENSG00000119866 | BCL11A | Hs.370549 | 363.1 | PP | 387.8 | PP |
| ENSG00000159388 | BTG2 | Hs.519162 | 588.2 | PP | 538.3 | PP |
| ENSG00000111913 | C6orf32 | Hs.484915 | 83.8 | PP | 62.8 | PP |
| ENSG00000168564 | CARF | Hs.32922 | 72.4 | PP | 130 | PP |
| ENSG00000130940 | CASZ1 | Hs.553537 | 255.7 | PP | 200 | PP |
| ENSG00000163660 | CCNL1 | Hs.4859 | 1082.6 | PP | 1052.5 | PP |
| ENSG00000070831 | CDC42 | Hs.467637 | 620.1 | PP | 437.75 | PP |
| ENSG00000153140 | CETN3 | Hs.528302 | 107.1 | PP | 199.4 | PP |
| ENSG00000122705 | CLTA | Hs.522114 | 1204.4 | PP | 990.1 | PP |
| ENSG00000108848 | CROP | Hs.130293 | 2166.4 | PP | 1637.55 | PP |
| ENSG00000048740 | CUGBP2 | Hs.309288 | 159.6 | PP | 334.5 | PP |
| ENSG00000036257 | CUL3 | Hs.372286 | 554.5 | PP | 473.25 | PP |
| ENSG00000121966 | CXCR4 | Hs.421986 | 532.4 | PP | 1919.4 | PP |
| ENSG00000112242 | E2F3 | Hs.269408 | 295.6 | PP | 585.95 | PP |
| ENSG00000134001 | EIF2S1 | Hs.151777 | 235.7 | PP | 299.55 | PP |
| ENSG00000104131 | EIF3S1 | Hs.404056 | 340.4 | PP | 365.05 | PP |
| ENSG00000110321 | EIF4G2 | Hs.183684 | 2137 | PP | 2701.9 | PP |
| ENSG00000066044 | ELAVL1 | Hs.184492 | 534.6 | PP | 734.45 | PP |
| ENSG00000120533 | ENY2 | Hs.492555 | 316.2 | PP | 375.85 | PP |
| ENSG00000120616 | EPC1 | Hs.167805 | 148.9 | PP | 88.7 | PA |
| ENSG00000157554 | ERG | Hs.473819 | 369.4 | PP | 298.05 | PA |
| ENSG00000120705 | ETF1 | Hs.483494 | 253.8 | PP | 461.55 | PP |
| ENSG00000119402 | FBXW2 | Hs.494985 | 157.2 | PP | 167.5 | PP |
| ENSG00000181495 | FLJ14054 | Hs.13528 | 1289.4 | PP | 771.75 | PP |
| ENSG00000170345 | FOS | Hs.25647 | 1254.3 | PP | 1832.65 | PP |
| ENSG00000145907 | G3BP | Hs.3353 | 167.7 | PP | 319.25 | PP |
| ENSG00000104064 | GABPB2 | Hs.546852 | 129.3 | PP | 183.75 | PM |
| ENSG00000196700 | GM632 | Hs.551552 | 103.5 | PP | 145.05 | PP |
| ENSG00000185245 | GP1BA | Hs.1472 | 274.2 | PP | 361.5 | PP |
| ENSG00000163349 | HIPK1 | Hs.532363 | 347.7 | PP | 488.5 | PP |
| ENSG00000122566 | HNRPA2B1 | Hs.487774 | 1802.2 | PP | 2268.3 | PP |
| ENSG00000170144 | HNRPA3 | Hs.516539 | 375.5 | PP | 558.6 | PP |
| ENSG00000138668 | HNRPD | Hs.480073 | 785.7 | PP | 1067.15 | PP |
| ENSG00000164164 | HSHIN1 | Hs.270851 | 303.1 | PP | 312.65 | PP |
| ENSG00000184216 | IRAK1 | Hs.522819 | 529.4 | PP | 607.85 | PP |
| ENSG00000115548 | JMJD1A | Hs.531819 | 264 | PP | 207.05 | PP |
| ENSG00000114030 | KPNA1 | Hs.161008 | 121.8 | PP | 91.95 | PP |
| ENSG00000166783 | LKAP | Hs.173524 | 135 | PP | 182.75 | PP |
| ENSG00000135387 | M11S1 | Hs.471818 | 464.9 | PP | 451.45 | PP |
| ENSG00000145495 | 6-Mar | Hs.432862 | 597.3 | PP | 536.05 | PP |
| ENSG00000168906 | MAT2A | Hs.516157 | 1011.4 | PP | 738.6 | PP |
| ENSG00000015479 | MATR3 | Hs.268939 | 252.4 | PP | 382.35 | PP |
| ENSG00000125952 | MAX | Hs.285354 | 1178.7 | PP | 1480.1 | PP |
| ENSG00000152601 | MBNL1 | Hs.478000 | 294.4 | PP | 864.5 | PP |
| ENSG00000124733 | MEA1 | Hs.278362 | 452.7 | PP | 460.2 | PP |
| ENSG00000133606 | MKRN1 | Hs.490347 | 392.7 | PP | 350.15 | PP |
| ENSG00000175727 | MONDOA | Hs.437153 | 201.1 | PP | 301.1 | PP |
| ENSG00000112651 | MRPL2 | Hs.55041 | 198.6 | PP | 110.3 | PA |
| ENSG00000138386 | NAB1 | Hs.107474 | 187.9 | PP | 203.9 | PP |
| ENSG00000082641 | NFE2L1 | Hs.514284 | 735.5 | PP | 726.35 | PP |
| ENSG00000106459 | NRF1 | Hs.298069 | 157.5 | PP | 126.8 | PA |
| ENSG00000046651 | OFD1 | Hs.6483 | 1022.7 | PP | 692.15 | PP |
| ENSG00000115421 | PAPOLG | Hs.387471 | 153.2 | PP | 91.25 | PP |
| ENSG00000169564 | PCBP1 | Hs.2853 | 1587.4 | PP | 1289.35 | PP |
| ENSG00000107242 | PIP5K1B | Hs.534371 | 279 | PP | 312.25 | PP |
| ENSG00000163806 | PPP1CB | Hs.468018 | 65.7 | PP | 135.05 | PP |
| ENSG00000112498 | PPP1R11 | Hs.82887 | 484.7 | PP | 463.3 | PP |
| ENSG00000107758 | PPP3CB | Hs.500067 | 318.9 | PP | 241.25 | PP |
| ENSG00000142875 | PRKACB | Hs.487325 | 1018.3 | PP | 994.45 | PP |
| ENSG00000143106 | PSMA5 | Hs.485246 | 649.7 | PP | 1036.3 | PP |
| ENSG00000108344 | PSMD3 | Hs.12970 | 156.3 | PP | 101.1 | PA |
| ENSG00000117569 | PTBP2 | Hs.269895 | 183 | PP | 160.15 | PP |
| ENSG00000112531 | QKI | Hs.510324 | 284.1 | PP | 564.45 | PP |
| ENSG00000075785 | RAB7 | Hs.15738 | 99.2 | PP | 107.25 | PP |
| ENSG00000077092 | RARB | Hs.436538 | 111.5 | PP | 156.85 | PP |
| ENSG00000067560 | RHOA | Hs.247077 | 1537.7 | PP | 2368.8 | PP |
| ENSG00000022840 | RNF10 | Hs.442798 | 327.5 | PP | 282.35 | PM |
| ENSG00000123091 | RNF11 | Hs.309641 | 125.3 | PP | 161.5 | PP |
| ENSG00000063978 | RNF4 | Hs.66394 | 115.8 | PP | 325.65 | PP |
| ENSG00000159216 | RUNX1 | Hs.149261 | 108.8 | PP | 211.2 | PP |
| ENSG00000052802 | SC4MOL | Hs.105269 | 146.9 | PP | 73.7 | PA |
| ENSG00000151967 | SCHIP1 | Hs.134665 | 497 | PP | 417.95 | PP |
| ENSG00000156642 | SDFR1 | Hs.187866 | 169.4 | PP | 193.05 | PP |
| ENSG00000107651 | SEC23IP | Hs.435004 | 115.6 | PP | 130.8 | PA |
| ENSG00000179833 | SERTAD2 | Hs.77293 | 498.4 | PP | 556.2 | PP |
| ENSG00000119335 | SET | Hs.436687 | 2967 | PP | 4307.1 | PP |
| ENSG00000136450 | SFRS1 | Hs.68714 | 1264.9 | PP | 1622.65 | PP |
| ENSG00000124193 | SFRS6 | Hs.6891 | 2001.1 | PP | 2200.75 | PP |
| ENSG00000096717 | SIRT1 | Hs.369779 | 133.8 | PP | 124.9 | PP |
| ENSG00000005022 | SLC25A5 | Hs.522767 | 4938.4 | PP | 5178.4 | PP |
| ENSG00000102038 | SMARCA1 | Hs.152292 | 101.1 | PP | 72.95 | PA |
| ENSG00000167088 | SNRPD1 | Hs.464734 | 518.7 | PP | 572.8 | PP |
| ENSG00000196700 | SOX18 | Hs.8619 | 1264.6 | PP | 2674 | PP |
| ENSG00000091640 | SPAG7 | Hs.90436 | 900.7 | PP | 517.9 | PP |
| ENSG00000152268 | SPON1 | Hs.445818 | 236.5 | PP | 121.5 | PP |
| ENSG00000180283 | SUMO2 | Hs.380973 | 1013.1 | PP | 783 | PP |
| ENSG00000178691 | SUZ12 | Hs.462732 | 602.2 | PP | 633.3 | PP |
| ENSG00000135316 | SYNCRIP | Hs.472056 | 178 | PP | 315.75 | PP |
| ENSG00000140416 | TPM1 | Hs.133892 | 394.4 | PP | 661.4 | PP |
| ENSG00000157514 | TSC22D3 | Hs.522074 | 1752.5 | PP | 2189.45 | PP |
| ENSG00000119048 | UBE2B | Hs.385986 | 335 | PP | 417.45 | PP |
| ENSG00000109332 | UBE2D3 | Hs.472031 | 471.2 | PP | 635.4 | PP |
| ENSG00000132670 | VPS16 | — | 333 | PP | 243.95 | PP |
| ENSG00000143952 | VPS54 | Hs.48499 | 187.9 | PP | 151.5 | PP |
| ENSG00000158195 | WASF2 | Hs.469244 | 430.1 | PP | 367.5 | PP |
| ENSG00000166913 | YWHAB | Hs.279920 | 222.4 | PP | 288.75 | PP |
| ENSG00000107372 | ZA20D2 | Hs.406096 | 967.2 | PP | 1262.9 | PP |
| ENSG00000186908 | ZDHHC17 | Hs.4014 | 123.5 | PP | 166.2 | PP |
| ENSG00000119725 | ZNF410 | Hs.270869 | 164.4 | PP | 257.1 | PP |

23a

| | | | | | | |
|---|---|---|---|---|---|---|
| ENSG00000137822 | 76P | Hs.549135 | 383.7 | PP | 259.6 | PP |
| ENSG00000075624 | ACTB | Hs.520640 | 11983.4 | PP | 11502.85 | PP |
| ENSG00000172493 | AFF1 | Hs.480190 | 480.6 | PP | 776.35 | PP |
| ENSG00000031003 | C5orf5 | Hs.483473 | 256.3 | PP | 316.85 | PP |
| ENSG00000135932 | CAB39 | Hs.471593 | 527.9 | PP | 637.3 | PP |
| ENSG00000100261 | CBX6 | Hs.511952 | 308 | PP | 255.7 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000135535 | CD164 | Hs.520313 | 2274.9 | PP | 2265.25 | PP |
| ENSG00000153774 | CFDP1 | Hs.461361 | 278.6 | PP | 295.4 | PP |
| ENSG00000111596 | CNOT2 | Hs.133350 | 431.4 | PP | 399.2 | PP |
| ENSG00000111605 | CPSF6 | Hs.369606 | 402.6 | PP | 474.05 | PP |
| ENSG00000009307 | CSDE1 | Hs.69855 | 1494.1 | PP | 1637.9 | PP |
| ENSG00000102974 | CTCF | Hs.368367 | 506.1 | PP | 535.55 | PP |
| ENSG00000048740 | CUGBP2 | Hs.309288 | 159.6 | PP | 334.5 | PP |
| ENSG00000036257 | CUL3 | Hs.372286 | 554.5 | PP | 473.25 | PP |
| ENSG00000124487 | DDX3X | Hs.380774 | 769.8 | PP | 1229 | PP |
| ENSG00000141141 | DDX52 | Hs.408452 | 124.3 | PP | 74.65 | PP |
| ENSG00000109606 | DHX15 | Hs.5683 | 1219.8 | PP | 1620.75 | PP |
| ENSG00000108406 | DHX40 | Hs.29403 | 233.3 | PP | 196.45 | PP |
| ENSG00000170522 | ELOVL6 | Hs.412939 | 96.5 | PP | 156.45 | PP |
| ENSG00000085832 | EPS15 | Hs.83722 | 144.1 | PP | 218.2 | PP |
| ENSG00000166147 | FBN1 | Hs.146447 | 195.2 | PP | 178.7 | PP |
| ENSG00000138081 | FBXO11 | Hs.553547 | 155.7 | PP | 259.35 | PP |
| ENSG00000114861 | FOXP1 | Hs.431498 | 322.7 | PP | 410.3 | PP |
| ENSG00000114416 | FXR1 | Hs.478407 | 143.9 | PP | 155.75 | PA |
| ENSG00000138757 | G3BP2 | Hs.303676 | 242.8 | PP | 428.55 | PP |
| ENSG00000166503 | HDGFRP3 | Hs.513954 | 139.2 | PP | 156 | PP |
| ENSG00000164104 | HMGB2 | Hs.434953 | 855.9 | PP | 2145.45 | PP |
| ENSG00000122566 | HNRPA2B1 | Hs.487774 | 1802.2 | PP | 2268.3 | PP |
| ENSG00000138668 | HNRPD | Hs.480073 | 785.7 | PP | 1067.15 | PP |
| ENSG00000126945 | HNRPH2 | Hs.278857 | 189.2 | PP | 158.45 | PP |
| ENSG00000099783 | HNRPM | Hs.465808 | 968.5 | PP | 880.2 | PP |
| ENSG00000125944 | HNRPR | Hs.373763 | 839.4 | PP | 980.1 | PP |
| ENSG00000153187 | HNRPU | Hs.166463 | 470.3 | PP | 887.6 | PP |
| ENSG00000120075 | HOXB5 | Hs.149548 | 322 | PP | 238.15 | PP |
| ENSG00000125347 | IRF1 | Hs.436061 | 467.7 | PP | 299.6 | PP |
| ENSG00000102753 | KPNA3 | Hs.527919 | 250.8 | PP | 391.35 | PP |
| ENSG00000185896 | LAMP1 | Hs.494419 | 282.2 | PP | 383.55 | PP |
| ENSG00000143815 | LBR | Hs.435166 | 1166 | PP | 1807.35 | PP |
| ENSG00000145012 | LPP | Hs.444362 | 205.6 | PP | 238.9 | PP |
| ENSG00000120992 | LYPLA1 | Hs.435850 | 84.5 | PP | 420.05 | PP |
| ENSG00000110514 | MADD | Hs.82548 | 423 | PP | 267.9 | PA |
| ENSG00000161021 | MAML1 | Hs.484349 | 331.3 | PP | 280.35 | PP |
| ENSG00000101367 | MAPRE1 | Hs.472437 | 196.1 | PP | 206.85 | PM |
| ENSG00000166974 | MAPRE2 | Hs.532824 | 857.4 | PP | 715.5 | PP |
| ENSG00000175130 | MARCKSL1 | Hs.75061 | 314.8 | PP | 265.6 | PP |
| ENSG00000164024 | METAP1 | Hs.480364 | 351.6 | PP | 422.25 | PP |
| ENSG00000187109 | NAP1L1 | Hs.524599 | 1185.6 | PP | 1531.65 | PP |
| ENSG00000166579 | NDEL1 | Hs.372123 | 218.3 | PP | 187.2 | PP |
| ENSG00000116044 | NFE2L2 | Hs.155396 | 181.2 | PP | 280.9 | PP |
| ENSG00000167770 | OTUB1 | Hs.473788 | 266 | PP | 155.35 | PA |
| ENSG00000059758 | PCTK2 | Hs.506415 | 118.3 | PP | 220.9 | PP |
| ENSG00000171033 | PKIA | Hs.433700 | 88.6 | PP | 131.05 | PP |
| ENSG00000146278 | PNRC1 | Hs.75969 | 316.4 | PP | 242.95 | PP |
| ENSG00000112941 | POLS | Hs.481542 | 355.5 | PP | 367.35 | PP |
| ENSG00000163806 | PPP1CB | Hs.468018 | 65.7 | PP | 135.05 | PP |
| ENSG00000107758 | PPP3CB | Hs.500067 | 318.9 | PP | 241.25 | PP |
| ENSG00000137492 | PRKRIR | Hs.503315 | 281.4 | PP | 549.7 | PP |
| ENSG00000120899 | PTK2B | Hs.491322 | 333.1 | PP | 183.65 | PP |
| ENSG00000184007 | PTP4A2 | Hs.470477 | 110.5 | PP | 254.55 | PP |
| ENSG00000134644 | PUM1 | Hs.281707 | 378.4 | PP | 422.3 | PP |
| ENSG00000112531 | QKI | Hs.510324 | 284.1 | PP | 564.45 | PP |
| ENSG00000175582 | RAB6A | Hs.503222 | 371.4 | PP | 365.75 | PP |
| ENSG00000119318 | RAD23B | Hs.521640 | 156.2 | PP | 211.05 | PP |
| ENSG00000065150 | RANBP5 | Hs.513057 | 123.7 | PP | 118.85 | PP |
| ENSG00000010017 | RANBP9 | Hs.306242 | 381.3 | PP | 489.95 | PP |
| ENSG00000127314 | RAP1B | Hs.369920 | 1093.1 | PP | 1537.75 | PP |
| ENSG00000103479 | RBL2 | Hs.513609 | 203.5 | PP | 188.25 | PP |
| ENSG00000134758 | RNF138 | Hs.302408 | 186.9 | PP | 511.9 | PP |
| ENSG00000163602 | RYBP | Hs.7910 | 398 | PP | 462.8 | PP |
| ENSG00000079332 | SARA1 | Hs.499960 | 233.7 | PP | 442.85 | PP |
| ENSG00000116350 | SFRS4 | Hs.76122 | 217.8 | PP | 375.35 | PP |
| ENSG00000115875 | SFRS7 | Hs.309090 | 378.7 | PP | 743.5 | PP |
| ENSG00000134294 | SLC38A2 | Hs.221847 | 634.3 | PP | 1026.55 | PP |
| ENSG00000008294 | SPAG9 | Hs.463439 | 180.3 | PP | 209.9 | PP |
| ENSG00000116030 | SUMO1 | Hs.81424 | 240.9 | PP | 268.85 | PP |
| ENSG00000106052 | TAX1BP1 | Hs.34576 | 550 | PP | 486.2 | PP |
| ENSG00000100207 | TCF20 | Hs.475018 | 251.3 | PP | 281.15 | PP |
| ENSG00000083312 | TNPO1 | Hs.482497 | 483 | PP | 750.85 | PP |
| ENSG00000077097 | TOP2B | Hs.475733 | 592.4 | PP | 806 | PP |
| ENSG00000166598 | TRA1 | Hs.192374 | 151 | PP | 339.35 | PP |
| ENSG00000140391 | TSPAN3 | Hs.5062 | 516.6 | PP | 661.45 | PP |
| ENSG00000109332 | UBE2D3 | Hs.472031 | 471.2 | PP | 635.4 | PP |
| ENSG00000135655 | USP15 | Hs.434951 | 197.3 | PP | 160.35 | PP |
| ENSG00000124486 | USP9X | Hs.77578 | 85.3 | PP | 115.1 | PP |
| ENSG00000078668 | VDAC3 | Hs.491597 | 3451.2 | PP | 4625.2 | PP |
| ENSG00000095787 | WAC | Hs.435610 | 379.2 | PP | 507.2 | PP |
| ENSG00000149658 | YTHDF1 | Hs.11747 | 710.9 | PP | 778.1 | PP |

23b

| | | | | | | |
|---|---|---|---|---|---|---|
| ENSG00000165029 | ABCA1 | Hs.429294 | 175.1 | PP | 156.5 | PP |
| ENSG00000173020 | ADRBK1 | Hs.83636 | 706 | PP | 549.3 | PM |
| ENSG00000172493 | AFF1 | Hs.480190 | 480.6 | PP | 776.35 | PP |
| ENSG00000131503 | ANKHD1 | Hs.434219 | 168 | PP | 163.05 | PP |
| ENSG00000062725 | APPBP2 | Hs.84084 | 106.4 | PP | 116.15 | PA |
| ENSG00000165527 | ARF6 | Hs.525330 | 444.1 | PP | 636.85 | PP |
| ENSG00000134108 | ARL10C | Hs.250009 | 578.1 | PP | 591.95 | PP |
| ENSG00000128989 | ARPP-19 | Hs.512908 | 247.7 | PP | 139.7 | PP |
| ENSG00000111875 | ASF1A | Hs.292316 | 126.5 | PP | 114.6 | PP |
| ENSG00000171456 | ASXL1 | Hs.374043 | 275.6 | PP | 307.25 | PP |
| ENSG00000017260 | ATP2C1 | Hs.546361 | 229.1 | PP | 322.9 | PP |
| ENSG00000147416 | ATP6V1B2 | Hs.295917 | 179.5 | PP | 183.85 | PP |
| ENSG00000105829 | BET1 | Hs.489132 | 150.8 | PP | 121.8 | PP |
| ENSG00000104765 | BNIP3L | Hs.131226 | 239.7 | PP | 375.45 | PP |
| ENSG00000136261 | BZW2 | Hs.487635 | 1167.4 | PP | 1664.45 | PP |
| ENSG00000152242 | C18orf25 | Hs.116486 | 129.7 | PP | 130.6 | PA |
| ENSG00000031003 | C5orf5 | Hs.483473 | 256.3 | PP | 316.85 | PP |
| ENSG00000104267 | CA2 | Hs.155097 | 119.4 | PP | 82.3 | PA |
| ENSG00000135932 | CAB39 | Hs.471593 | 527.9 | PP | 637.3 | PP |
| ENSG00000113328 | CCNG1 | Hs.79101 | 1025 | PP | 737.8 | PP |
| ENSG00000134480 | CCNH | Hs.292524 | 651.2 | PP | 697.05 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000153774 | CFDP1 | Hs.461361 | 278.6 | PP | 295.4 | PP |
| ENSG00000109572 | CLCN3 | Hs.481186 | 244.9 | PP | 248.15 | PP |
| ENSG00000111596 | CNOT2 | Hs.133350 | 431.4 | PP | 399.2 | PA |
| ENSG00000111605 | CPSF6 | Hs.369606 | 402.6 | PP | 474.05 | PP |
| ENSG00000101266 | CSNK2A1 | Hs.446484 | 170.4 | PP | 261.4 | PP |
| ENSG00000102974 | CTCF | Hs.368367 | 506.1 | PP | 535.55 | PP |
| ENSG00000171793 | CTPS | Hs.473087 | 206.2 | PP | 242.75 | PP |
| ENSG00000048740 | CUGBP2 | Hs.309288 | 159.6 | PP | 334.5 | PP |
| ENSG00000036257 | CUL3 | Hs.372286 | 554.5 | PP | 473.25 | PP |
| ENSG00000165659 | DACH1 | Hs.129452 | 95.1 | PP | 144.8 | PP |
| ENSG00000124487 | DDX3X | Hs.380774 | 769.8 | PP | 1229 | PP |
| ENSG00000141141 | DDX52 | Hs.408452 | 124.3 | PP | 74.65 | PP |
| ENSG00000109606 | DHX15 | Hs.5683 | 1219.8 | PP | 1620.75 | PP |
| ENSG00000152128 | DKFZP566N034 | Hs.369471 | 538.7 | PP | 502.55 | PP |
| ENSG00000198121 | EDG2 | Hs.126667 | 243.1 | PP | 147 | PA |
| ENSG00000124802 | EEF1E1 | Hs.88977 | 196.2 | PP | 308 | PA |
| ENSG00000171570 | EGLN2 | Hs.515417 | 259 | PP | 194.45 | PM |
| ENSG00000120738 | EGR1 | Hs.326035 | 128.6 | PP | 290.15 | PP |
| ENSG00000107581 | EIF3S10 | Hs.523299 | 522.9 | PP | 586.55 | PP |
| ENSG00000156976 | EIF4A2 | Hs.478553 | 3569 | PP | 6526.55 | PP |
| ENSG00000131503 | EIF4EBP3 /// | — | 285.5 | PP | 200 | PP |
| | MASK-BP3 | | | | | |
| ENSG00000109381 | ELF2 | Hs.260903 | 150.8 | PP | 152.4 | PP |
| ENSG00000102034 | ELF4 | Hs.271940 | 309.2 | PP | 202.7 | PP |
| ENSG00000012660 | ELOVL5 | Hs.520189 | 681.6 | PP | 667.3 | PP |
| ENSG00000170522 | ELOVL6 | Hs.412939 | 96.5 | PP | 156.45 | PP |
| ENSG00000085832 | EPS15 | Hs.83722 | 144.1 | PP | 218.2 | PP |
| ENSG00000120705 | ETF1 | Hs.483494 | 253.8 | PP | 461.55 | PP |
| ENSG00000166147 | FBN1 | Hs.146447 | 195.2 | PP | 178.7 | PP |
| ENSG00000138081 | FBXO11 | Hs.553547 | 155.7 | PP | 259.35 | PP |
| ENSG00000181495 | FLJ14054 | Hs.13528 | 1289.4 | PP | 771.75 | PP |
| ENSG00000136122 | FLJ22624 | Hs.209715 | 121.6 | PP | 115.85 | PP |
| ENSG00000125740 | FOSB | Hs.75678 | 1751.3 | PP | 3599.55 | PP |
| ENSG00000138757 | G3BP2 | Hs.303676 | 242.8 | PP | 428.55 | PP |
| ENSG00000146729 | GBAS | Hs.437367 | 186.8 | PP | 145.8 | PP |
| ENSG00000115419 | GLS | Hs.116448 | 217.6 | PP | 242.2 | PP |
| ENSG00000166503 | HDGFRP3 | Hs.513954 | 139.2 | PP | 156 | PP |
| ENSG00000010818 | HIVEP2 | Hs.510172 | 149.6 | PP | 157.9 | PP |
| ENSG00000100281 | HMG2L1 | Hs.197086 | 215.2 | PP | 87.75 | PM |
| ENSG00000164104 | HMGB2 | Hs.434953 | 855.9 | PP | 2145.45 | PP |
| ENSG00000122566 | HNRPA2B1 | Hs.487774 | 1802.2 | PP | 2268.3 | PP |
| ENSG00000092199 | HNRPC | Hs.449114 | 926.9 | PP | 1161.55 | PP |
| ENSG00000138668 | HNRPD | Hs.480073 | 785.7 | PP | 1067.15 | PP |
| ENSG00000152795 | HNRPDL | Hs.527105 | 1931.8 | PP | 1802.05 | PP |
| ENSG00000096746 | HNRPH3 | Hs.499891 | 408.7 | PP | 635.5 | PP |
| ENSG00000165119 | HNRPK | Hs.449171 | 761 | PP | 1269.35 | PP |
| ENSG00000125944 | HNRPR | Hs.373763 | 839.4 | PP | 980.1 | PP |
| ENSG00000153187 | HNRPU | Hs.166463 | 470.3 | PP | 887.6 | PP |
| ENSG00000120075 | HOXB5 | Hs.149548 | 322 | PP | 238.15 | PP |
| ENSG00000173744 | HRB | Hs.443507 | 202.6 | PP | 405.7 | PP |
| ENSG00000164164 | HSHIN1 | Hs.270851 | 303.1 | PP | 312.65 | PP |
| ENSG00000144381 | HSPD1 | Hs.113684 | 2868.6 | PP | 5689.3 | PP |
| ENSG00000136478 | HT008 | Hs.175414 | 443.2 | PP | 336.2 | PP |
| ENSG00000086758 | HUWE1 | Hs.136905 | 535.9 | PP | 503.6 | PP |
| ENSG00000068383 | INPP5A | Hs.523360 | 302.3 | PP | 279.95 | PP |
| ENSG00000125347 | IRF1 | Hs.436061 | 467.7 | PP | 299.6 | PP |
| ENSG00000171988 | JMJD1C | Hs.413416 | 107 | PP | 198.85 | PP |
| ENSG00000126814 | KIAA1393 | Hs.380159 | 1228.6 | PP | 1731.35 | PP |
| ENSG00000156232 | KIAA1971 /// | Hs.377360 | 75.3 | PP | 71.35 | PP |
| | LOC339005 /// | | | | | |
| | LOC440253 | | | | | |
| ENSG00000165516 | KLHDC2 | Hs.509264 | 709.9 | PP | 490.3 | PP |
| ENSG00000102753 | KPNA3 | Hs.527919 | 250.8 | PP | 391.35 | PP |
| ENSG00000108424 | KPNB1 | Hs.532793 | 415.1 | PP | 709.85 | PP |
| ENSG00000108424 | KPNB1 | Hs.532793 | 160.1 | PP | 294.95 | PP |
| ENSG00000185896 | LAMP1 | Hs.494419 | 282.2 | PP | 383.55 | PP |
| ENSG00000197017 | LAT1-3TM /// | Hs.444600 | 269.6 | PP | 389.55 | PP |
| | BOLA2 /// | | | | | |
| | LOC595101 | | | | | |
| ENSG00000143815 | LBR | Hs.435166 | 1166 | PP | 1807.35 | PP |
| ENSG00000157978 | LDLRAP1 | Hs.184482 | 223.5 | PP | 298.4 | PP |
| ENSG00000113368 | LMNB1 | Hs.89497 | 75.4 | PP | 143.6 | PA |
| ENSG00000123684 | LPGAT1 | Hs.497674 | 106 | PP | 114.3 | PP |
| ENSG00000145012 | LPP | Hs.444362 | 205.6 | PP | 238.9 | PP |
| ENSG00000120992 | LYPLA1 | Hs.435850 | 84.5 | PP | 420.05 | PP |
| ENSG00000003056 | M6PR | Hs.134084 | 566.2 | PP | 575.55 | PP |
| ENSG00000127603 | MACF1 | Hs.472475 | 222.8 | PP | 248.35 | PP |
| ENSG00000110514 | MADD | Hs.82548 | 423 | PP | 267.9 | PA |
| ENSG00000121579 | MAK3 | Hs.269528 | 268.8 | PP | 307 | PP |
| ENSG00000161021 | MAML1 | Hs.484349 | 331.3 | PP | 280.35 | PP |
| ENSG00000197442 | MAP3K5 | Hs.186486 | 227.6 | PP | 272.25 | PP |
| ENSG00000055208 | MAP3K7IP2 | Hs.269775 | 219.1 | PP | 142.8 | PP |
| ENSG00000101367 | MAPRE1 | Hs.472437 | 196.1 | PP | 206.85 | PM |
| ENSG00000166974 | MAPRE2 | Hs.532824 | 857.4 | PP | 715.5 | PP |
| ENSG00000175130 | MARCKSL1 | Hs.75061 | 314.8 | PP | 265.6 | PP |
| ENSG00000168906 | MAT2A | Hs.516157 | 1011.4 | PP | 738.6 | PP |
| ENSG00000015479 | MATR3 | Hs.268939 | 252.4 | PP | 382.35 | PP |
| ENSG00000152601 | MBNL1 | Hs.478000 | 294.4 | PP | 864.5 | PP |
| ENSG00000180398 | MCFD2 | Hs.293689 | 289.4 | PP | 258.25 | PP |
| ENSG00000081189 | MEF2C | Hs.444409 | 426 | PP | 437.85 | PP |
| ENSG00000143995 | MEIS1 | Hs.526754 | 452.1 | PP | 262.65 | PP |
| ENSG00000164024 | METAP1 | Hs.480364 | 351.6 | PP | 422.25 | PP |
| ENSG00000198408 | MGEA5 | Hs.500842 | 363.7 | PP | 363.05 | PP |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000187109 | NAP1L1 | Hs.524599 | 1185.6 | PP | 1531.65 | PP |
| ENSG00000198646 | NCOA6 | Hs.368971 | 334.7 | PP | 445.5 | PP |
| ENSG00000166579 | NDEL1 | Hs.372123 | 218.3 | PP | 187.2 | PP |
| ENSG00000116044 | NFE2L2 | Hs.155396 | 181.2 | PP | 280.9 | PP |
| ENSG00000181163 | NPM1 | Hs.519452 | 8905.6 | PP | 11897.7 | PP |
| ENSG00000180530 | NRIP1 | Hs.155017 | 842 | PP | 914.85 | PP |
| ENSG00000147162 | OGT | Hs.405410 | 165.1 | PP | 183.6 | PP |
| ENSG00000091039 | OSBPL8 | Hs.430849 | 212 | PP | 251.65 | PP |
| ENSG00000167770 | OTUB1 | Hs.473788 | 266 | PP | 155.35 | PA |
| ENSG00000172939 | OXSR1 | Hs.475970 | 211 | PP | 186.15 | PA |
| ENSG00000059758 | PCTK2 | Hs.506415 | 118.3 | PP | 220.9 | PP |
| ENSG00000150593 | PDCD4 | Hs.232543 | 174.3 | PP | 122.55 | PP |
| ENSG00000143870 | PDIA6 | Hs.212102 | 357.6 | PP | 852.3 | PP |
| ENSG00000073921 | PICALM | Hs.163893 | 357.3 | PP | 315.6 | PP |
| ENSG00000171033 | PKIA | Hs.433700 | 88.6 | PP | 131.05 | PP |
| ENSG00000118495 | PLAGL1 | Hs.444975 | 187.1 | PP | 357.8 | PP |
| ENSG00000100941 | PNN | Hs.409965 | 839.5 | PP | 617.3 | PP |
| ENSG00000146278 | PNRC1 | Hs.75969 | 316.4 | PP | 242.95 | PP |
| ENSG00000163806 | PPP1CB | Hs.468018 | 65.7 | PP | 135.05 | PP |
| ENSG00000058272 | PPP1R12A | Hs.49582 | 154.9 | PP | 178.75 | PP |
| ENSG00000154001 | PPP2R5E | Hs.334868 | 135.1 | PP | 191.45 | PP |
| ENSG00000116731 | PRDM2 | Hs.371823 | 329.6 | PP | 175.6 | PP |
| ENSG00000166501 | PRKCB1 | Hs.460355 | 742.5 | PP | 535.05 | PP |
| ENSG00000137492 | PRKRIR | Hs.503315 | 281.4 | PP | 549.7 | PP |
| ENSG00000147224 | PRPS1 | Hs.56 | 508.2 | PP | 485.95 | PP |
| ENSG00000171522 | PTGER4 | Hs.199248 | 531.8 | PP | 514.8 | PP |
| ENSG00000184007 | PTP4A2 | Hs.470477 | 110.5 | PP | 254.55 | PP |
| ENSG00000134644 | PUM1 | Hs.281707 | 378.4 | PP | 422.3 | PP |
| ENSG00000055917 | PUM2 | Hs.467824 | 238.6 | PP | 313.25 | PP |
| ENSG00000112531 | QKI | Hs.510324 | 284.1 | PP | 564.45 | PP |
| ENSG00000048991 | R3HDM | Hs.412462 | 217.6 | PP | 376.75 | PP |
| ENSG00000119396 | RAB14 | Hs.371563 | 140.5 | PP | 198.15 | PP |
| ENSG00000111737 | RAB35 | Hs.524788 | 394.6 | PP | 431.2 | PP |
| ENSG00000152942 | RAD17 | Hs.16184 | 57.2 | PP | 60 | PM |
| ENSG00000164754 | RAD21 | Hs.81848 | 281.3 | PP | 414.9 | PP |
| ENSG00000119318 | RAD23B | Hs.521640 | 156.2 | PP | 211.05 | PP |
| ENSG00000065150 | RANBP5 | Hs.513057 | 123.7 | PP | 118.85 | PP |
| ENSG00000010017 | RANBP9 | Hs.306242 | 381.3 | PP | 489.95 | PP |
| ENSG00000116473 | RAP1A | Hs.190334 | 217.2 | PP | 367.75 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000119707 | RBM25 | Hs.531106 | 231.5 | PP | 319.9 | PP |
| ENSG00000049449 | RCN1 | Hs.97887 | 294.1 | PP | 264.85 | PP |
| ENSG00000176624 | RKHD2 | Hs.465144 | 210.8 | PP | 216 | PP |
| ENSG00000124782 | RREB1 | Hs.298248 | 339.9 | PP | 324.15 | PP |
| ENSG00000137815 | RTF1 | Hs.511096 | 194.3 | PP | 256.85 | PP |
| ENSG00000163602 | RYBP | Hs.7910 | 398 | PP | 462.8 | PP |
| ENSG00000160633 | SAFB | Hs.23978 | 376.8 | PP | 261.35 | PP |
| ENSG00000130254 | SAFB2 | Hs.546330 | 245.2 | PP | 213.5 | PP |
| ENSG00000182568 | SATB1 | Hs.517717 | 829.9 | PP | 652.05 | PP |
| ENSG00000137575 | SDCBP | Hs.200804 | 934 | PP | 955.25 | PP |
| ENSG00000155878 | SEC22L1 | Hs.534212 | 662.9 | PP | 588.95 | PP |
| ENSG00000119335 | SET | Hs.436687 | 2967 | PP | 4307.1 | PP |
| ENSG00000136450 | SFRS1 | Hs.68714 | 1264.9 | PP | 1622.65 | PP |
| ENSG00000116350 | SFRS4 | Hs.76122 | 217.8 | PP | 375.35 | PP |
| ENSG00000115875 | SFRS7 | Hs.309090 | 378.7 | PP | 743.5 | PP |
| ENSG00000198730 | SH2BP1 | Hs.173288 | 215.7 | PP | 203.65 | PP |
| ENSG00000162105 | SHANK2 | Hs.268726 | 137.1 | PP | 128.55 | PP |
| ENSG00000004864 | SLC25A13 | Hs.489190 | 106.7 | PP | 139.8 | PP |
| ENSG00000134294 | SLC38A2 | Hs.221847 | 634.3 | PP | 1026.55 | PP |
| ENSG00000080493 | SLC4A4 | Hs.5462 | 124.5 | PP | 268.4 | PP |
| ENSG00000072501 | SMC1L1 | Hs.211602 | 137.5 | PP | 329.1 | PP |
| ENSG00000102172 | SMS | Hs.529887 | 287.8 | PP | 275.25 | PP |
| ENSG00000163788 | SNRK | Hs.476052 | 320.6 | PP | 436.65 | PP |
| ENSG00000008294 | SPAG9 | Hs.463439 | 180.3 | PP | 209.9 | PP |
| ENSG00000065526 | SPEN | Hs.270499 | 257.6 | PP | 262.7 | PP |
| ENSG00000147475 | SPFH2 | Hs.125849 | 125.1 | PP | 180.9 | PP |
| ENSG00000173757 | STAT5B | Hs.132864 | 463.6 | PP | 373 | PP |
| ENSG00000168952 | STXBP6 | Hs.508958 | 152.6 | PP | 218.55 | PP |
| ENSG00000173812 | SUI1 | Hs.150580 | 6046 | PP | 6098.85 | PP |
| ENSG00000116030 | SUMO1 | Hs.81424 | 240.9 | PP | 268.85 | PP |
| ENSG00000106052 | TAX1BP1 | Hs.34576 | 550 | PP | 486.2 | PP |
| ENSG00000114126 | TFDP2 | Hs.379018 | 253.2 | PP | 196.3 | PA |
| ENSG00000177426 | TGIF | Hs.373550 | 116.7 | PP | 95.35 | PM |
| ENSG00000198586 | TLK1 | Hs.470586 | 150.2 | PP | 117.65 | PP |
| ENSG00000120802 | TMPO | Hs.11355 | 206.3 | PP | 161.7 | PP |
| ENSG00000083312 | TNPO1 | Hs.482497 | 483 | PP | 750.85 | PP |
| ENSG00000183864 | TOB2 | Hs.474978 | 165.3 | PP | 196.4 | PP |
| ENSG00000198900 | TOP1 | Hs.472737 | 315.5 | PP | 468.35 | PP |
| ENSG00000077097 | TOP2B | Hs.475733 | 592.4 | PP | 806 | PP |
| ENSG00000166598 | TRA1 | Hs.192374 | 151 | PP | 339.35 | PP |
| ENSG00000173334 | TRIB1 | Hs.444947 | 238.3 | PP | 300.85 | PP |
| ENSG00000140391 | TSPAN3 | Hs.5062 | 516.6 | PP | 661.45 | PP |
| ENSG00000131508 | UBE2D2 | Hs.108332 | 400.1 | PP | 512.05 | PP |
| ENSG00000109332 | UBE2D3 | Hs.472031 | 471.2 | PP | 635.4 | PP |
| ENSG00000188021 | UBQLN2 | Hs.522668 | 112.4 | PP | 145.55 | PP |
| ENSG00000169021 | UQCRFS1 | Hs.170107 | 1584.8 | PP | 1518.7 | PP |
| ENSG00000124486 | USP9X | Hs.77578 | 85.3 | PP | 115.1 | PP |
| ENSG00000101558 | VAPA | Hs.165195 | 1051.8 | PP | 1198.45 | PP |
| ENSG00000155959 | VBP1 | Hs.436803 | 330 | PP | 595.4 | PP |
| ENSG00000105053 | VRK3 | Hs.443330 | 276.1 | PP | 148 | PA |
| ENSG00000095787 | WAC | Hs.435610 | 379.2 | PP | 507.2 | PP |
| ENSG00000109046 | WSB1 | Hs.446017 | 679.3 | PP | 880.75 | PP |
| ENSG00000146457 | WTAP | Hs.446091 | 400.8 | PP | 406.6 | PP |
| ENSG00000149658 | YTHDF1 | Hs.11747 | 710.9 | PP | 778.1 | PP |
| ENSG00000100811 | YY1 | Hs.388927 | 539.7 | PP | 740.2 | PP |
| ENSG00000086666 | ZA20D3 | Hs.306329 | 735.1 | PP | 517.75 | PP |
| ENSG00000185650 | ZFP36L1 | Hs.85155 | 94.5 | PP | 140.7 | PP |
| ENSG00000179456 | ZNF238 | Hs.69997 | 235.7 | PP | 241.8 | PP |
| ENSG00000121848 | ZNF364 | Hs.523550 | 205.2 | PP | 286.9 | PP |
| ENSG00000005955 | ZNF403 | Hs.514116 | 382 | PP | 264.25 | PP |
| ENSG00000036549 | ZZZ3 | Hs.480506 | 125 | PP | 189.7 | PP |

26a

| | | | | | | |
|---|---|---|---|---|---|---|
| ENSG00000165029 | ABCA1 | Hs.429294 | 175.1 | PP | 156.5 | PP |
| ENSG00000125257 | ABCC4 | Hs.508423 | 301.5 | PP | 348.15 | PP |
| ENSG00000101126 | ADNP | Hs.293736 | 552.6 | PP | 494.1 | PP |
| ENSG00000173020 | ADRBK1 | Hs.83636 | 706 | PP | 549.3 | PM |
| ENSG00000172493 | AFF1 | Hs.480190 | 480.6 | PP | 776.35 | PP |
| ENSG00000155189 | AGPAT5 | Hs.490899 | 207.1 | PP | 241.35 | PP |
| ENSG00000151320 | AKAP6 | Hs.509083 | 137.8 | PP | 117.05 | PP |
| ENSG00000143761 | ARF1 | Hs.286221 | 1245.2 | PP | 798.4 | PP |
| ENSG00000117713 | ARID1A | Hs.468972 | 477.6 | PP | 444.8 | PP |
| ENSG00000188042 | ARL7 | Hs.111554 | 240.7 | PP | 203.4 | PP |
| ENSG00000163466 | ARPC2 | Hs.529303 | 1447.8 | PP | 1774.7 | PP |
| ENSG00000128989 | ARPP-19 | Hs.512908 | 247.7 | PP | 139.7 | PP |
| ENSG00000171456 | ASXL1 | Hs.374043 | 275.6 | PP | 307.25 | PP |
| ENSG00000149311 | ATM | Hs.435561 | 221.9 | PP | 205.5 | PP |
| ENSG00000017260 | ATP2C1 | Hs.546361 | 229.1 | PP | 322.9 | PP |
| ENSG00000155096 | AZIN1 | Hs.459106 | 551.2 | PP | 619.2 | PP |
| ENSG00000119866 | BCL11A | Hs.370549 | 363.1 | PP | 387.8 | PP |
| ENSG00000134107 | BHLHB2 | Hs.171825 | 482.8 | PP | 341.5 | PP |
| ENSG00000133639 | BTG1 | Hs.255935 | 955.9 | PP | 1057.95 | PP |
| ENSG00000134153 | C15orf24 | Hs.160565 | 612.5 | PP | 706.2 | PP |
| ENSG00000152242 | C18orf25 | Hs.116486 | 129.7 | PP | 130.6 | PA |
| ENSG00000101084 | C20orf24 | Hs.184062 | 878.9 | PP | 1348.2 | PP |
| ENSG00000114405 | C3orf14 | Hs.47166 | 131.5 | PP | 86.85 | PA |
| ENSG00000168476 | C8orf20 | Hs.289063 | 470.1 | PP | 364.3 | PA |
| ENSG00000135837 | CAP350 | Hs.413045 | 177.4 | PP | 220.9 | PP |
| ENSG00000116489 | CAPZA1 | Hs.514934 | 766.7 | PP | 1165.8 | PA |
| ENSG00000077549 | CAPZB | Hs.432760 | 282.7 | PP | 199.2 | PP |
| ENSG00000142453 | CARM1 | Hs.371416 | 141.6 | PP | 105.7 | PA |
| ENSG00000067955 | CBFB | Hs.460988 | 719.1 | PP | 1032 | PP |
| ENSG00000118971 | CCND2 | Hs.376071 | 1114.1 | PP | 1244.6 | PP |
| ENSG00000135535 | CD164 | Hs.520313 | 2274.9 | PP | 2265.25 | PP |
| ENSG00000070831 | CDC42 | Hs.467637 | 620.1 | PP | 437.75 | PP |
| ENSG00000111328 | CDK2AP1 | Hs.433201 | 1539.3 | PP | 1576.75 | PP |
| ENSG00000072609 | CHFR | Hs.507336 | 247.4 | PP | 253.05 | PP |
| ENSG00000110172 | CHORDC1 | Hs.22857 | 84.8 | PP | 156.6 | PP |
| ENSG00000131873 | CHSY1 | Hs.110488 | 404.5 | PP | 490.95 | PP |
| ENSG00000141367 | CLTC | Hs.491351 | 1169 | PP | 1399.6 | PP |
| ENSG00000162368 | CMPK | Hs.11463 | 333.8 | PP | 336.55 | PP |
| ENSG00000100528 | CNIH | Hs.294603 | 252.6 | PP | 391.7 | PP |
| ENSG00000166200 | COPS2 | Hs.369614 | 521.2 | PP | 700.65 | PP |
| ENSG00000178741 | COX5A | Hs.401903 | 808.6 | PP | 1092.9 | PP |
| ENSG00000005339 | CREBBP | Hs.459759 | 287.2 | PP | 239.6 | PP |
| ENSG00000149187 | CUGBP1 | Hs.530727 | 383 | PP | 596.3 | PP |
| ENSG00000048740 | CUGBP2 | Hs.309288 | 159.6 | PP | 334.5 | PP |
| ENSG00000196730 | DAPK1 | Hs.380277 | 448.2 | PP | 549.6 | PP |
| ENSG00000124487 | DDX3X | Hs.380774 | 769.8 | PP | 1229 | PP |
| ENSG00000067048 | DDX3Y | Hs.99120 | 302.6 | PP | 309.6 | PA |
| ENSG00000108654 | DDX5 | Hs.279806 | 3052 | PP | 3508.95 | PP |
| ENSG00000155792 | DEPDC6 | Hs.303788 | 461.4 | PP | 409.4 | PP |
| ENSG00000109606 | DHX15 | Hs.5683 | 1219.8 | PP | 1620.75 | PP |
| ENSG00000100697 | DICER1 | Hs.87889 | 176.8 | PP | 186.3 | PP |
| ENSG00000151208 | DLG5 | Hs.500245 | 73.1 | PP | 90.5 | PP |
| ENSG00000092964 | DPYSL2 | Hs.173381 | 505.1 | PP | 495.1 | PP |
| ENSG00000157540 | DYRK1A | Hs.368240 | 367.8 | PP | 751.2 | PP |
| ENSG00000110047 | EHD1 | Hs.523774 | 293.2 | PP | 330.3 | PP |
| ENSG00000110321 | EIF4G2 | Hs.183684 | 2137 | PP | 2701.9 | PP |
| ENSG00000100664 | EIF5 | Hs.433702 | 96.4 | PP | 55.8 | PA |
| ENSG00000120616 | EPC1 | Hs.167805 | 148.9 | PP | 88.7 | PA |
| ENSG00000120705 | ETF1 | Hs.483494 | 253.8 | PP | 461.55 | PP |
| ENSG00000106462 | EZH2 | Hs.444082 | 162.9 | PP | 216.25 | PP |
| ENSG00000119979 | FAM45B /// | Hs.434241 | 554.8 | PP | 690.2 | PP |
| | FAM45A | | | | | |
| ENSG00000153310 | FAM49B | Hs.492869 | 311.6 | PP | 329.6 | PP |
| ENSG00000138081 | FBXO11 | Hs.553547 | 155.7 | PP | 259.35 | PP |
| ENSG00000143756 | FBXO28 | Hs.64691 | 106.1 | PP | 70.5 | PA |
| ENSG00000037637 | FBXO42 | Hs.522384 | 136.1 | PP | 120.85 | PM |
| ENSG00000111011 | FLJ11021 | Hs.432996 | 202 | PP | 392.6 | PP |
| ENSG00000136048 | FLJ11259 | Hs.525634 | 351.2 | PP | 243.8 | PP |
| ENSG00000175606 | FLJ20533 | Hs.106650 | 420.6 | PP | 454.55 | PP |
| ENSG00000114861 | FOXP1 | Hs.431498 | 322.7 | PP | 410.3 | PP |
| ENSG00000138757 | G3BP2 | Hs.303676 | 242.8 | PP | 428.55 | PP |
| ENSG00000106070 | GRB10 | Hs.164060 | 550.7 | PP | 574.15 | PP |
| ENSG00000103342 | GSPT1 | Hs.528780 | 356.7 | PP | 369.6 | PP |
| ENSG00000103342 | GSPT1 | Hs.528780 | 275.4 | PP | 515.9 | PP |
| ENSG00000110768 | GTF2H1 | Hs.523510 | 167.7 | PP | 191.7 | PP |
| ENSG00000115207 | GTF3C2 | Hs.75782 | 405.3 | PP | 405 | PA |
| ENSG00000164032 | H2AFZ | Hs.119192 | 1477 | PP | 3477.65 | PP |
| ENSG00000114315 | HES1 | Hs.250666 | 232.7 | PP | 169.8 | PP |
| ENSG00000163349 | HIPK1 | Hs.532363 | 347.7 | PP | 488.5 | PP |
| ENSG00000118418 | HMGN3 | Hs.77558 | 1438.8 | PP | 1894.65 | PP |
| ENSG00000177733 | HNRPA0 | Hs.96996 | 655 | PP | 723.4 | PP |
| ENSG00000105323 | HNRPUL1 | Hs.155218 | 406.9 | PP | 401.15 | PP |
| ENSG00000106004 | HOXA5 | Hs.37034 | 507.2 | PP | 499.3 | PP |
| ENSG00000164164 | HSHIN1 | Hs.270851 | 303.1 | PP | 312.65 | PP |
| ENSG00000086758 | HUWE1 | Hs.136905 | 535.9 | PP | 503.6 | PP |
| ENSG00000071243 | ING3 | Hs.489811 | 255.5 | PP | 407 | PP |
| ENSG00000143127 | ITGA10 | Hs.158237 | 145 | PP | 142.3 | PM |
| ENSG00000008083 | JARID2 | Hs.269059 | 1015 | PP | 894 | PP |
| ENSG00000164151 | KIAA0947 | Hs.449296 | 282.9 | PP | 275.45 | PP |
| ENSG00000155090 | KLF10 | Hs.435001 | 348.9 | PP | 357.65 | PP |
| ENSG00000136826 | KLF4 | Hs.376206 | 536.5 | PP | 1193.7 | PP |
| ENSG00000102753 | KPNA3 | Hs.527919 | 250.8 | PP | 391.35 | PP |
| ENSG00000107929 | LARP5 | Hs.498455 | 274.8 | PP | 203.9 | PP |
| ENSG00000145012 | LPP | Hs.444362 | 205.6 | PP | 238.9 | PP |
| ENSG00000127603 | MACF1 | Hs.472475 | 222.8 | PP | 248.35 | PP |
| ENSG00000111885 | MAN1A1 | Hs.102788 | 309.1 | PP | 151 | PP |
| ENSG00000166963 | MAP1A | Hs.194301 | 433.9 | PP | 321.4 | PM |
| ENSG00000055208 | MAP3K7IP2 | Hs.269775 | 219.1 | PP | 142.8 | PP |
| ENSG00000135525 | MAP7 | Hs.486548 | 325.7 | PP | 390.1 | PP |
| ENSG00000166986 | MARS | Hs.355867 | 460.7 | PP | 614.95 | PP |
| ENSG00000015479 | MATR3 | Hs.268939 | 252.4 | PP | 382.35 | PP |
| ENSG00000133858 | MGC23401 | Hs.527874 | 291 | PP | 309.55 | PP |
| ENSG00000108788 | MLX | Hs.383019 | 219 | PP | 195.3 | PP |
| ENSG00000114978 | MOBK1B | Hs.196437 | 596.3 | PP | 556 | PP |
| ENSG00000123562 | MORF4L2 | Hs.326387 | 2189.8 | PP | 2626.1 | PP |
| ENSG00000147649 | MTDH | Hs.377155 | 258.6 | PP | 329.8 | PP |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000128654 | MTX2 | Hs.470728 | 121.8 | PP | 135.85 | PP |
| ENSG00000138386 | NAB1 | Hs.107474 | 187.9 | PP | 203.9 | PP |
| ENSG00000189043 | NDUFA4 | Hs.50098 | 1613.7 | PP | 1432.5 | PP |
| ENSG00000050344 | NFE2L3 | Hs.404741 | 94.5 | PP | 84.4 | PP |
| ENSG00000120837 | NFYB | Hs.84928 | 204.1 | PP | 272.85 | PP |
| ENSG00000164190 | NIPBL | Hs.481927 | 165.1 | PP | 145.15 | PP |
| ENSG00000180530 | NRIP1 | Hs.155017 | 842 | PP | 914.85 | PP |
| ENSG00000137497 | NUMA1 | Hs.523873 | 486.3 | PP | 295.45 | PA |
| ENSG00000124789 | NUP153 | Hs.121088 | 216.4 | PP | 291.8 | PP |
| ENSG00000065154 | OAT | Hs.523332 | 304 | PP | 486.9 | PP |
| ENSG00000147162 | OGT | Hs.405410 | 165.1 | PP | 183.6 | PP |
| ENSG00000117859 | OSBPL9 | Hs.21938 | 534.9 | PP | 419.5 | PP |
| ENSG00000100266 | PACSIN2 | Hs.162877 | 717.7 | PP | 302.25 | PP |
| ENSG00000142864 | PAI-RBP1 | Hs.530412 | 337.8 | PP | 662.45 | PP |
| ENSG00000180370 | PAK2 | Hs.518530 | 147.3 | PP | 198.15 | PP |
| ENSG00000105835 | PBEF1 | Hs.489615 | 96.8 | PP | 138.65 | PP |
| ENSG00000114054 | PCCB | Hs.63788 | 270.6 | PP | 392.75 | PP |
| ENSG00000114209 | PDCD10 | Hs.478150 | 463.6 | PP | 560.4 | PP |
| ENSG00000184588 | PDE4B | Hs.198072 | 234.1 | PP | 349.5 | PP |
| ENSG00000164040 | PGRMC2 | Hs.507910 | 89.1 | PP | 87.85 | PM |
| ENSG00000118482 | PHF3 | Hs.348921 | 260 | PP | 268.9 | PP |
| ENSG00000145675 | PIK3R1 | Hs.132225 | 249.1 | PP | 181 | PP |
| ENSG00000137193 | PIM1 | Hs.81170 | 1021.7 | PP | 801.85 | PP |
| ENSG00000154217 | PITPNC1 | Hs.549130 | 391.3 | PP | 316.6 | PP |
| ENSG00000181191 | PJA1 | Hs.522679 | 316.3 | PP | 332.1 | PP |
| ENSG00000152952 | PLOD2 | Hs.477866 | 92.1 | PP | 128.6 | PP |
| ENSG00000004399 | PLXND1 | Hs.301685 | 578.6 | PP | 512.35 | PP |
| ENSG00000146278 | PNRC1 | Hs.75969 | 316.4 | PP | 242.95 | PP |
| ENSG00000078304 | PPP2R5C | Hs.368264 | 174.9 | PP | 165.25 | PP |
| ENSG00000138814 | PPP3CA | Hs.435512 | 122.5 | PP | 205 | PP |
| ENSG00000107758 | PPP3CB | Hs.500067 | 318.9 | PP | 241.25 | PP |
| ENSG00000154845 | PPP4R1 | Hs.464595 | 472.6 | PP | 378.85 | PP |
| ENSG00000131238 | PPT1 | Hs.3873 | 767.9 | PP | 861.2 | PP |
| ENSG00000117569 | PTBP2 | Hs.269895 | 183 | PP | 160.15 | PP |
| ENSG00000171862 | PTEN | Hs.500466 | 353.7 | PP | 334.4 | PP |
| ENSG00000110958 | PTGES3 | Hs.50425 | 1018.1 | PP | 1526.7 | PP |
| ENSG00000112245 | PTP4A1 | Hs.227777 | 439.5 | PP | 431.2 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000103769 | RAB11A | Hs.321541 | 1087.7 | PP | 1020.15 | PP |
| ENSG00000017797 | RALBP1 | Hs.528993 | 379 | PP | 244.05 | PP |
| ENSG00000010017 | RANBP9 | Hs.306242 | 381.3 | PP | 489.95 | PP |
| ENSG00000116473 | RAP1A | Hs.190334 | 217.2 | PP | 367.75 | PP |
| ENSG00000139687 | RB1 | Hs.408528 | 152.7 | PP | 153.35 | PP |
| ENSG00000102054 | RBBP7 | Hs.495755 | 942.6 | PP | 1356.35 | PP |
| ENSG00000136144 | RCBTB1 | Hs.508021 | 121.9 | PP | 194.4 | PP |
| ENSG00000049449 | RCN1 | Hs.97887 | 294.1 | PP | 264.85 | PP |
| ENSG00000089902 | RCOR1 | Hs.510521 | 118.3 | PP | 144.05 | PP |
| ENSG00000176624 | RKHD2 | Hs.465144 | 210.8 | PP | 216 | PP |
| ENSG00000123091 | RNF11 | Hs.309641 | 125.3 | PP | 161.5 | PP |
| ENSG00000134758 | RNF138 | Hs.302408 | 186.9 | PP | 511.9 | PP |
| ENSG00000127870 | RNF6 | Hs.136885 | 74.1 | PP | 122.9 | PP |
| ENSG00000131051 | RNPC2 | Hs.282901 | 1756.3 | PP | 1736.7 | PP |
| ENSG00000137815 | RTF1 | Hs.511096 | 194.3 | PP | 256.85 | PP |
| ENSG00000163602 | RYBP | Hs.7910 | 398 | PP | 462.8 | PP |
| ENSG00000163785 | RYK | Hs.245869 | 232.7 | PP | 184.9 | PP |
| ENSG00000092421 | SEMA6A | Hs.156967 | 140 | PP | 182.2 | PP |
| ENSG00000120742 | SERP1 | Hs.518326 | 285.7 | PP | 343.4 | PP |
| ENSG00000153914 | SFRS12 | Hs.519347 | 258 | PP | 229.6 | PP |
| ENSG00000161547 | SFRS2 | Hs.73965 | 3723.5 | PP | 4390.4 | PP |
| ENSG00000116350 | SFRS4 | Hs.76122 | 217.8 | PP | 375.35 | PP |
| ENSG00000122912 | SLC25A16 | Hs.180408 | 693.5 | PP | 585.25 | PP |
| ENSG00000178537 | SLC25A20 | Hs.13845 | 513.4 | PP | 284.75 | PP |
| ENSG00000134294 | SLC38A2 | Hs.221847 | 634.3 | PP | 1026.55 | PP |
| ENSG00000080493 | SLC4A4 | Hs.5462 | 124.5 | PP | 268.4 | PP |
| ENSG00000175387 | SMAD2 | Hs.12253 | 243.4 | PP | 321.4 | PP |
| ENSG00000184602 | SNN | Hs.459952 | 234.4 | PP | 239.3 | PP |
| ENSG00000163788 | SNRK | Hs.476052 | 320.6 | PP | 436.65 | PP |
| ENSG00000089006 | SNX5 | Hs.316890 | 327.9 | PP | 403.25 | PP |
| ENSG00000124766 | SOX4 | Hs.357901 | 2351.7 | PP | 2500.65 | PP |
| ENSG00000095480 | SPFH1 | Hs.150087 | 646.4 | PP | 689.15 | PP |
| ENSG00000115306 | SPTBN1 | Hs.503178 | 589.9 | PP | 344.65 | PP |
| ENSG00000153037 | SRP19 | Hs.483090 | 290.8 | PP | 365.35 | PP |
| ENSG00000096063 | SRPK1 | Hs.443861 | 231.6 | PP | 200.5 | PP |
| ENSG00000198648 | STK39 | Hs.276271 | 129.8 | PP | 111.35 | PP |
| ENSG00000117632 | STMN1 | Hs.209983 | 546.6 | PP | 663.4 | PP |
| ENSG00000165283 | STOML2 | Hs.3439 | 880.1 | PP | 583.05 | PP |
| ENSG00000140262 | TCF12 | Hs.511504 | 179.4 | PP | 439.15 | PP |
| ENSG00000123066 | THRAP2 | Hs.159799 | 142.1 | PP | 396.2 | PP |
| ENSG00000100926 | TM9SF1 | Hs.91586 | 214.5 | PP | 148.05 | PP |
| ENSG00000170348 | TMP21 | Hs.74137 | 293.5 | PP | 280.3 | PA |
| ENSG00000198900 | TOP1 | Hs.472737 | 315.5 | PP | 468.35 | PP |
| ENSG00000165699 | TSC1 | Hs.370854 | 232.7 | PP | 242.3 | PP |
| ENSG00000163084 | TSN | Hs.75066 | 130.8 | PP | 178.35 | PP |
| ENSG00000077721 | UBE2A | Hs.379466 | 299.1 | PP | 400.85 | PP |
| ENSG00000131508 | UBE2D2 | Hs.108332 | 400.1 | PP | 512.05 | PP |
| ENSG00000109332 | UBE2D3 | Hs.472031 | 471.2 | PP | 635.4 | PP |
| ENSG00000132388 | UBE2G1 | Hs.462035 | 272.5 | PP | 373.45 | PP |
| ENSG00000185651 | UBE2L3 | Hs.108104 | 775.4 | PP | 605.6 | PP |
| ENSG00000124208 | UBE2V1 /// Kua- | Hs.420529 | 504.3 | PP | 320.6 | PP |
| | UEV | | | | | |
| ENSG00000114062 | UBE3A | Hs.22543 | 456.9 | PP | 470.75 | PP |
| ENSG00000130939 | UBE4B | Hs.386404 | 291.8 | PP | 387.95 | PP |
| ENSG00000135655 | USP15 | Hs.434951 | 197.3 | PP | 160.35 | PP |
| ENSG00000115464 | USP34 | Hs.468663 | 1075.5 | PP | 1012.75 | PP |
| ENSG00000090686 | USP48 | Hs.467524 | 366.1 | PP | 393.65 | PP |
| ENSG00000117533 | VAMP4 | Hs.6651 | 271.8 | PP | 299.3 | PP |
| ENSG00000136485 | WDR68 | Hs.410596 | 150.9 | PP | 192.1 | PP |
| ENSG00000108953 | YWHAE | Hs.513851 | 328.6 | PP | 424.95 | PP |
| ENSG00000108953 | YWHAE | Hs.513851 | 2306.8 | PP | 4150.1 | PP |
| ENSG00000086666 | ZA20D3 | Hs.306329 | 735.1 | PP | 517.75 | PP |
| ENSG00000023041 | ZDHHC6 | Hs.196990 | 289.3 | PP | 293.9 | PP |
| ENSG00000179456 | ZNF238 | Hs.69997 | 235.7 | PP | 241.8 | PP |

26b

| | | | | | | |
|---|---|---|---|---|---|---|
| ENSG00000115091 | ACTR3 | Hs.433512 | 353.8 | PP | 673.3 | PP |
| ENSG00000101126 | ADNP | Hs.293736 | 552.6 | PP | 494.1 | PP |
| ENSG00000143761 | ARF1 | Hs.286221 | 1245.2 | PP | 798.4 | PP |
| ENSG00000163466 | ARPC2 | Hs.529303 | 1447.8 | PP | 1774.7 | PP |
| ENSG00000155096 | AZIN1 | Hs.459106 | 551.2 | PP | 619.2 | PP |
| ENSG00000134107 | BHLHB2 | Hs.171825 | 482.8 | PP | 341.5 | PP |
| ENSG00000134153 | C15orf24 | Hs.160565 | 612.5 | PP | 706.2 | PP |
| ENSG00000114405 | C3orf14 | Hs.47166 | 131.5 | PP | 86.85 | PA |
| ENSG00000077549 | CAPZB | Hs.432760 | 282.7 | PP | 199.2 | PP |
| ENSG00000082258 | CCNT2 | Hs.292754 | 114.4 | PP | 102.75 | PP |
| ENSG00000072609 | CHFR | Hs.507336 | 247.4 | PP | 253.05 | PP |
| ENSG00000100528 | CNIH | Hs.294603 | 252.6 | PP | 391.7 | PP |
| ENSG00000166200 | COPS2 | Hs.369614 | 521.2 | PP | 700.65 | PP |
| ENSG00000005339 | CREBBP | Hs.459759 | 287.2 | PP | 239.6 | PP |
| ENSG00000149187 | CUGBP1 | Hs.530727 | 383 | PP | 596.3 | PP |
| ENSG00000108654 | DDX5 | Hs.279806 | 3052 | PP | 3508.95 | PP |
| ENSG00000104131 | EIF3S1 | Hs.404056 | 340.4 | PP | 365.05 | PP |
| ENSG00000156976 | EIF4A2 | Hs.478553 | 3569 | PP | 6526.55 | PP |
| ENSG00000120616 | EPC1 | Hs.167805 | 148.9 | PP | 88.7 | PA |
| ENSG00000120705 | ETF1 | Hs.483494 | 253.8 | PP | 461.55 | PP |
| ENSG00000120699 | EXOSC8 | Hs.294041 | 196.9 | PP | 283.25 | PP |
| ENSG00000106462 | EZH2 | Hs.444082 | 162.9 | PP | 216.25 | PP |
| ENSG00000138081 | FBXO11 | Hs.553547 | 155.7 | PP | 259.35 | PP |
| ENSG00000114861 | FOXP1 | Hs.431498 | 322.7 | PP | 410.3 | PP |
| ENSG00000106070 | GRB10 | Hs.164060 | 550.7 | PP | 574.15 | PP |
| ENSG00000103342 | GSPT1 | Hs.528780 | 356.7 | PP | 369.6 | PA |
| ENSG00000164032 | H2AFZ | Hs.119192 | 1477 | PP | 3477.65 | PP |
| ENSG00000114315 | HES1 | Hs.250666 | 232.7 | PP | 169.8 | PP |
| ENSG00000029993 | HMGB3 | Hs.19114 | 309 | PP | 460.6 | PP |
| ENSG00000177733 | HNRPA0 | Hs.96996 | 655 | PP | 723.4 | PP |
| ENSG00000096746 | HNRPH3 | Hs.499891 | 408.7 | PP | 635.5 | PP |
| ENSG00000106004 | HOXA5 | Hs.37034 | 507.2 | PP | 499.3 | PP |
| ENSG00000067829 | IDH3G | Hs.410197 | 249.2 | PP | 156.95 | PA |
| ENSG00000002834 | LASP1 | Hs.334851 | 786.5 | PP | 659.05 | PP |
| ENSG00000145012 | LPP | Hs.444362 | 205.6 | PP | 238.9 | PP |
| ENSG00000135387 | M11S1 | Hs.471818 | 464.9 | PP | 451.45 | PP |
| ENSG00000127603 | MACF1 | Hs.472475 | 222.8 | PP | 248.35 | PP |
| ENSG00000111885 | MAN1A1 | Hs.102788 | 309.1 | PP | 151 | PP |
| ENSG00000108788 | MLX | Hs.383019 | 219 | PP | 195.3 | PP |
| ENSG00000123562 | MORF4L2 | Hs.326387 | 2189.8 | PP | 2626.1 | PP |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000120837 | NFYB | Hs.84928 | 204.1 | PP | 272.85 | PP |
| ENSG00000164190 | NIPBL | Hs.481927 | 165.1 | PP | 145.15 | PP |
| ENSG00000180530 | NRIP1 | Hs.155017 | 842 | PP | 914.85 | PP |
| ENSG00000137497 | NUMA1 | Hs.523873 | 486.3 | PP | 295.45 | PA |
| ENSG00000100266 | PACSIN2 | Hs.162877 | 717.7 | PP | 302.25 | PP |
| ENSG00000180370 | PAK2 | Hs.518530 | 147.3 | PP | 198.15 | PP |
| ENSG00000105835 | PBEF1 | Hs.489615 | 96.8 | PP | 138.65 | PP |
| ENSG00000118482 | PHF3 | Hs.348921 | 260 | PP | 268.9 | PP |
| ENSG00000166037 | PIG8 | Hs.101014 | 356.2 | PP | 374.1 | PP |
| ENSG00000051382 | PIK3CB | Hs.239818 | 275.1 | PP | 280.95 | PP |
| ENSG00000137193 | PIM1 | Hs.81170 | 1021.7 | PP | 801.85 | PP |
| ENSG00000181191 | PJA1 | Hs.522679 | 316.3 | PP | 332.1 | PP |
| ENSG00000004399 | PLXND1 | Hs.301685 | 578.6 | PP | 512.35 | PP |
| ENSG00000146278 | PNRC1 | Hs.75969 | 316.4 | PP | 242.95 | PP |
| ENSG00000078304 | PPP2R5C | Hs.368264 | 174.9 | PP | 165.25 | PP |
| ENSG00000131238 | PPT1 | Hs.3873 | 767.9 | PP | 861.2 | PP |
| ENSG00000110958 | PTGES3 | Hs.50425 | 1018.1 | PP | 1526.7 | PP |
| ENSG00000112245 | PTP4A1 | Hs.227777 | 439.5 | PP | 431.2 | PP |
| ENSG00000134644 | PUM1 | Hs.281707 | 378.4 | PP | 422.3 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000119318 | RAD23B | Hs.521640 | 156.2 | PP | 211.05 | PP |
| ENSG00000017797 | RALBP1 | Hs.528993 | 379 | PP | 244.05 | PP |
| ENSG00000010017 | RANBP9 | Hs.306242 | 381.3 | PP | 489.95 | PP |
| ENSG00000109756 | RAPGEF2 | Hs.113912 | 394.9 | PP | 551.95 | PP |
| ENSG00000139687 | RB1 | Hs.408528 | 152.7 | PP | 153.35 | PP |
| ENSG00000102054 | RBBP7 | Hs.495755 | 942.6 | PP | 1356.35 | PP |
| ENSG00000171700 | RGS19 | Hs.422336 | 164.1 | PP | 279.7 | PP |
| ENSG00000123091 | RNF11 | Hs.309641 | 125.3 | PP | 161.5 | PP |
| ENSG00000137075 | RNF38 | Hs.333503 | 322.6 | PP | 227.7 | PP |
| ENSG00000163602 | RYBP | Hs.7910 | 398 | PP | 462.8 | PP |
| ENSG00000168758 | SEMA4C | Hs.516220 | 186.7 | PP | 157.95 | PP |
| ENSG00000092421 | SEMA6A | Hs.156967 | 140 | PP | 182.2 | PP |
| ENSG00000153914 | SFRS12 | Hs.519347 | 258 | PP | 229.6 | PP |
| ENSG00000161547 | SFRS2 | Hs.73965 | 3723.5 | PP | 4390.4 | PP |
| ENSG00000116350 | SFRS4 | Hs.76122 | 217.8 | PP | 375.35 | PP |
| ENSG00000122912 | SLC25A16 | Hs.180408 | 693.5 | PP | 585.25 | PP |
| ENSG00000178537 | SLC25A20 | Hs.13845 | 513.4 | PP | 284.75 | PP |
| ENSG00000134294 | SLC38A2 | Hs.221847 | 634.3 | PP | 1026.55 | PP |
| ENSG00000089006 | SNX5 | Hs.316890 | 327.9 | PP | 403.25 | PP |
| ENSG00000124766 | SOX4 | Hs.357901 | 2351.7 | PP | 2500.65 | PP |
| ENSG00000008294 | SPAG9 | Hs.463439 | 180.3 | PP | 209.9 | PP |
| ENSG00000095480 | SPFH1 | Hs.150087 | 646.4 | PP | 689.15 | PP |
| ENSG00000115306 | SPTBN1 | Hs.503178 | 589.9 | PP | 344.65 | PP |
| ENSG00000153037 | SRP19 | Hs.483090 | 290.8 | PP | 365.35 | PP |
| ENSG00000096063 | SRPK1 | Hs.443861 | 231.6 | PP | 200.5 | PP |
| ENSG00000124783 | SSR1 | Hs.114033 | 600.7 | PP | 686.65 | PP |
| ENSG00000165283 | STOML2 | Hs.3439 | 880.1 | PP | 583.05 | PP |
| ENSG00000170348 | TMP21 | Hs.74137 | 293.5 | PP | 280.3 | PA |
| ENSG00000091164 | TXNL1 | Hs.114412 | 1326.6 | PP | 1354.5 | PP |
| ENSG00000077721 | UBE2A | Hs.379466 | 299.1 | PP | 400.85 | PP |
| ENSG00000131508 | UBE2D2 | Hs.108332 | 400.1 | PP | 512.05 | PP |
| ENSG00000114062 | UBE3A | Hs.22543 | 456.9 | PP | 470.75 | PP |
| ENSG00000130939 | UBE4B | Hs.386404 | 291.8 | PP | 387.95 | PP |
| ENSG00000152518 | ZFP36L2 | Hs.503093 | 4553.7 | PP | 5205.6 | PP |
| ENSG00000010244 | ZNF207 | Hs.500775 | 918.9 | PP | 742.3 | PP |
| ENSG00000179456 | ZNF238 | Hs.69997 | 235.7 | PP | 241.8 | PP |

155

| | | | | | | |
|---|---|---|---|---|---|---|
| ENSG00000108599 | AKAP10 | Hs.462457 | 164.1 | PP | 228.75 | PP |
| ENSG00000170017 | ALCAM | Hs.150693 | 528.9 | PP | 695.45 | PP |
| ENSG00000123505 | AMD1 | Hs.159118 | 648.3 | PP | 1011.85 | PP |
| ENSG00000144848 | APG3L | Hs.477126 | 235.6 | PP | 245.05 | PP |
| ENSG00000111875 | ASF1A | Hs.292316 | 126.5 | PP | 114.6 | PP |
| ENSG00000143153 | ATP1B1 | Hs.291196 | 132.4 | PP | 245.55 | PP |
| ENSG00000155096 | AZIN1 | Hs.459106 | 551.2 | PP | 619.2 | PP |
| ENSG00000119866 | BCL11A | Hs.370549 | 363.1 | PP | 387.8 | PP |
| ENSG00000171791 | BCL2 | Hs.150749 | 151.5 | PP | 134.45 | PP |
| ENSG00000141452 | C18orf8 | Hs.529006 | 232.8 | PP | 242.05 | PP |
| ENSG00000135932 | CAB39 | Hs.471593 | 527.9 | PP | 637.3 | PP |
| ENSG00000108349 | CASC3 | Hs.350229 | 286.5 | PP | 317.4 | PP |
| ENSG00000196776 | CD47 | Hs.446414 | 407.8 | PP | 514.4 | PP |
| ENSG00000196776 | CD47 | Hs.446414 | 1026.3 | PP | 1175.5 | PP |
| ENSG00000172216 | CEBPB | Hs.517106 | 374.1 | PP | 391 | PP |
| ENSG00000177200 | CHD9 | Hs.530936 | 222.6 | PP | 263 | PP |
| ENSG00000100528 | CNIH | Hs.294603 | 252.6 | PP | 391.7 | PP |
| ENSG00000141030 | COPS3 | Hs.6076 | 331 | PP | 374.8 | PP |
| ENSG00000005339 | CREBBP | Hs.459759 | 287.2 | PP | 239.6 | PP |
| ENSG00000108848 | CROP | Hs.130293 | 2166.4 | PP | 1637.55 | PP |
| ENSG00000048740 | CUGBP2 | Hs.309288 | 159.6 | PP | 334.5 | PP |
| ENSG00000160967 | CUTL1 | Hs.438974 | 333.3 | PP | 412.55 | PP |
| ENSG00000168209 | DDIT4 | Hs.523012 | 736.8 | PP | 535.85 | PP |
| ENSG00000108406 | DHX40 | Hs.29403 | 233.3 | PP | 196.45 | PP |
| ENSG00000113391 | DKFZP564D172 | Hs.127788 | 94 | PP | 123.9 | PP |
| ENSG00000132002 | DNAJB1 | Hs.515210 | 453.4 | PP | 2770.05 | PA |
| ENSG00000128590 | DNAJB9 | Hs.6790 | 211.5 | PP | 433.55 | PP |
| ENSG00000110047 | EHD1 | Hs.523774 | 293.2 | PP | 330.3 | PP |
| ENSG00000120705 | ETF1 | Hs.483494 | 253.8 | PP | 461.55 | PP |
| ENSG00000138081 | FBXO11 | Hs.553547 | 155.7 | PP | 259.35 | PP |
| ENSG00000170345 | FOS | Hs.25647 | 1254.3 | PP | 1832.65 | PP |
| ENSG00000114416 | FXR1 | Hs.478407 | 143.9 | PP | 155.75 | PP |
| ENSG00000138757 | G3BP2 | Hs.303676 | 242.8 | PP | 428.55 | PP |
| ENSG00000034713 | GABARAPL2 | Hs.461379 | 952 | PP | 625.75 | PP |
| ENSG00000100644 | HIF1A | Hs.509554 | 866.5 | PP | 1037.1 | PP |
| ENSG00000010818 | HIVEP2 | Hs.510172 | 149.6 | PP | 157.9 | PP |
| ENSG00000122566 | HNRPA2B1 | Hs.487774 | 1802.2 | PP | 2268.3 | PP |
| ENSG00000153187 | HNRPU | Hs.166463 | 470.3 | PP | 887.6 | PP |
| ENSG00000113263 | ITK | Hs.483938 | 85.1 | PP | 57.35 | PP |
| ENSG00000008083 | JARID2 | Hs.269059 | 1015 | PP | 894 | PP |
| ENSG00000115548 | JMJD1A | Hs.531819 | 264 | PP | 207.05 | PP |
| ENSG00000177606 | JUN | Hs.525704 | 1300.5 | PP | 2286.95 | PP |
| ENSG00000170852 | KBTBD2 | Hs.372541 | 412.2 | PP | 381.65 | PP |
| ENSG00000055118 | KCNH2 | Hs.188021 | 592.3 | PP | 951 | PP |
| ENSG00000114030 | KPNA1 | Hs.161008 | 121.8 | PP | 91.95 | PP |
| ENSG00000055208 | MAP3K7IP2 | Hs.269775 | 219.1 | PP | 142.8 | PP |
| ENSG00000012983 | MAP4K5 | Hs.130491 | 66 | PP | 116 | PP |
| ENSG00000101367 | MAPRE1 | Hs.472437 | 196.1 | PP | 206.85 | PM |
| ENSG00000168906 | MAT2A | Hs.516157 | 1011.4 | PP | 738.6 | PP |
| ENSG00000015479 | MATR3 | Hs.268939 | 252.4 | PP | 382.35 | PP |
| ENSG00000143995 | MEIS1 | Hs.526754 | 452.1 | PP | 262.65 | PP |
| ENSG00000056586 | MNAB | Hs.533499 | 117.9 | PP | 127.15 | PP |
| ENSG00000005810 | MYCBP2 | Hs.151411 | 187 | PP | 643.55 | PP |
| ENSG00000164190 | NIPBL | Hs.481927 | 165.1 | PP | 145.15 | PP |
| ENSG00000147162 | OGT | Hs.405410 | 165.1 | PP | 183.6 | PP |
| ENSG00000145730 | PAM | Hs.369430 | 187.3 | PP | 178.45 | PP |
| ENSG00000090060 | PAPOLA | Hs.253726 | 307.4 | PP | 286 | PP |
| ENSG00000113387 | PC4 | Hs.229641 | 700.5 | PP | 1225.75 | PP |
| ENSG00000168283 | PCGF4 | Hs.380403 | 254.7 | PP | 304.1 | PP |
| ENSG00000059758 | PCTK2 | Hs.506415 | 118.3 | PP | 220.9 | PP |
| ENSG00000184588 | PDE4B | Hs.198072 | 234.1 | PP | 349.5 | PP |
| ENSG00000184588 | PDE4B | Hs.198072 | 119.8 | PP | 88.95 | PP |
| ENSG00000197329 | PELI1 | Hs.7886 | 83.3 | PP | 154.5 | PP |
| ENSG00000073921 | PICALM | Hs.163893 | 357.3 | PP | 315.6 | PP |
| ENSG00000171033 | PKIA | Hs.433700 | 88.6 | PP | 131.05 | PP |
| ENSG00000136040 | PLXNC1 | Hs.551522 | 354.6 | PP | 387.1 | PP |
| ENSG00000185246 | PRPF39 | Hs.274337 | 63.4 | PP | 107.2 | PP |
| ENSG00000117569 | PTBP2 | Hs.269895 | 183 | PP | 160.15 | PP |
| ENSG00000112531 | QKI | Hs.510324 | 284.1 | PP | 564.45 | PP |
| ENSG00000138069 | RAB1A | Hs.310645 | 70.8 | PP | 109.75 | PM |
| ENSG00000175582 | RAB6A | Hs.503222 | 371.4 | PP | 365.75 | PP |
| ENSG00000119318 | RAD23B | Hs.521640 | 156.2 | PP | 211.05 | PP |
| ENSG00000109756 | RAPGEF2 | Hs.113912 | 394.9 | PP | 551.95 | PP |
| ENSG00000089902 | RCOR1 | Hs.510521 | 118.3 | PP | 144.05 | PP |
| ENSG00000173039 | RELA | Hs.502875 | 389 | PP | 393.5 | PP |
| ENSG00000106615 | RHEB | Hs.283521 | 943.5 | PP | 1172.5 | PP |
| ENSG00000177105 | RHOG | Hs.501728 | 536.1 | PP | 466.25 | PP |
| ENSG00000132383 | RPA1 | Hs.461925 | 256 | PP | 464.2 | PP |
| ENSG00000124782 | RREB1 | Hs.298248 | 339.9 | PP | 324.15 | PP |
| ENSG00000182568 | SATB1 | Hs.517717 | 829.9 | PP | 652.05 | PP |
| ENSG00000137575 | SDCBP | Hs.200804 | 934 | PP | 955.25 | PP |
| ENSG00000138758 | 11-Sep | Hs.128199 | 350.4 | PP | 559 | PP |
| ENSG00000179833 | SERTAD2 | Hs.77293 | 498.4 | PP | 556.2 | PP |
| ENSG00000136450 | SFRS1 | Hs.68714 | 1264.9 | PP | 1622.65 | PP |
| ENSG00000136527 | SFRS10 | Hs.533122 | 673.5 | PP | 894.8 | PP |
| ENSG00000155926 | SLA | Hs.75367 | 495.3 | PP | 494.3 | PP |
| ENSG00000127616 | SMARCA4 | Hs.327527 | 238 | PP | 345.6 | PP |
| ENSG00000115904 | SOS1 | Hs.278733 | 144 | PP | 169.4 | PP |
| ENSG00000172845 | SP3 | Hs.531587 | 137.8 | PP | 293.35 | PP |
| ENSG00000135250 | SRPK2 | Hs.285197 | 106.2 | PP | 118.7 | PM |
| ENSG00000178691 | SUZ12 | Hs.462732 | 602.2 | PP | 633.3 | PP |
| ENSG00000008282 | SYPL1 | Hs.80919 | 157.3 | PP | 190.55 | PP |
| ENSG00000135090 | TAOK3 | — | 518.8 | PP | 394 | PP |
| ENSG00000114126 | TFDP2 | Hs.379018 | 253.2 | PP | 196.3 | PA |
| ENSG00000123066 | THRAP2 | Hs.159799 | 142.1 | PP | 396.2 | PP |
| ENSG00000106537 | TM4SF13 | Hs.364544 | 275.4 | PP | 526.45 | PP |
| ENSG00000173726 | TOMM20 | Hs.533192 | 656.5 | PP | 888.45 | PP |
| ENSG00000109332 | UBE2D3 | Hs.472031 | 471.2 | PP | 635.4 | PP |
| ENSG00000077254 | USP33 | Hs.480597 | 228.7 | PP | 299.75 | PP |
| ENSG00000119801 | YPEL5 | Hs.515890 | 1377.9 | PP | 1469.25 | PP |
| ENSG00000108953 | YWHAE | Hs.513851 | 328.6 | PP | 424.95 | PP |
| ENSG00000164924 | YWHAZ | Hs.492407 | 363.6 | PP | 520.8 | PP |
| ENSG00000107372 | ZA20D2 | Hs.406096 | 967.2 | PP | 1262.9 | PP |
| ENSG00000086666 | ZA20D3 | Hs.306329 | 735.1 | PP | 517.75 | PP |
| ENSG00000179456 | ZNF238 | Hs.69997 | 235.7 | PP | 241.8 | PP |

222

| | | | | | | |
|---|---|---|---|---|---|---|
| ENSG00000114770 | ABCC5 | Hs.368563 | 373.4 | PP | 322.1 | PM |
| ENSG00000084234 | APLP2 | Hs.370247 | 1126.7 | PP | 668.55 | PP |
| ENSG00000084234 | APLP2 | Hs.370247 | 343.4 | PP | 262.35 | PP |
| ENSG00000117713 | ARID1A | Hs.468972 | 477.6 | pp | 444.8 | PP |
| ENSG00000163399 | ATP1A1 | Hs.371889 | 370.4 | PP | 539.1 | PP |
| ENSG00000154640 | BTG3 | Hs.473420 | 315.6 | PP | 378.5 | PP |
| ENSG00000111596 | CNOT2 | Hs.133350 | 431.4 | PP | 399.2 | PP |
| ENSG00000108848 | CROP | Hs.130293 | 2166.4 | PP | 1637.55 | PP |
| ENSG00000149187 | CUGBP1 | Hs.530727 | 383 | PP | 596.3 | PP |
| ENSG00000160967 | CUTL1 | Hs.438974 | 333.3 | PP | 412.55 | PP |
| ENSG00000124487 | DDX3X | Hs.380774 | 769.8 | PP | 1229 | PP |
| ENSG00000104131 | EIF3S1 | Hs.404056 | 340.4 | PP | 365.05 | PP |
| ENSG00000110321 | EIF4G2 | Hs.183684 | 2137 | PP | 2701.9 | PP |
| ENSG00000139146 | FAM60A | Hs.505154 | 598.1 | PP | 788 | PP |
| ENSG00000138081 | FBXO11 | Hs.553547 | 155.7 | PP | 259.35 | PP |
| ENSG00000132589 | FLOT2 | Hs.514038 | 393.4 | PP | 355.45 | PP |
| ENSG00000170345 | FOS | Hs.25647 | 1254.3 | PP | 1832.65 | PP |
| ENSG00000165879 | FRAT1 | Hs.126057 | 207.4 | PP | 183.35 | PP |
| ENSG00000138668 | HNRPD | Hs.480073 | 785.7 | PP | 1067.15 | PP |
| ENSG00000173744 | HRB | Hs.443507 | 202.6 | PP | 405.7 | PP |
| ENSG00000171988 | JMJD1C | Hs.413416 | 107 | PP | 198.85 | PP |
| ENSG00000176407 | KCMF1 | Hs.345694 | 751.2 | PP | 938.3 | PP |
| ENSG00000168906 | MAT2A | Hs.516157 | 1011.4 | PP | 738.6 | PP |
| ENSG00000038274 | MAT2B | Hs.54642 | 609.4 | PP | 461.9 | PP |
| ENSG00000143995 | MEIS1 | Hs.526754 | 452.1 | PP | 262.65 | PP |
| ENSG00000125445 | MRPS7 | Hs.71787 | 381.8 | PP | 338.3 | PA |
| ENSG00000153234 | NR4A2 | Hs.165258 | 124.6 | PP | 242.85 | PP |
| ENSG00000059758 | PCTK2 | Hs.506415 | 118.3 | PP | 220.9 | PP |
| ENSG00000114209 | PDCD10 | Hs.478150 | 463.6 | PP | 560.4 | PP |
| ENSG00000168291 | PDHB | Hs.161357 | 453.5 | PP | 670.7 | PP |
| ENSG00000112511 | PHF1 | Hs.166204 | 903.7 | PP | 604.1 | PP |
| ENSG00000118495 | PLAGL1 | Hs.444975 | 187.1 | PP | 357.8 | PP |
| ENSG00000136040 | PLXNC1 | Hs.551522 | 354.6 | PP | 387.1 | PP |
| ENSG00000163806 | PPP1CB | Hs.468018 | 65.7 | PP | 135.05 | PP |
| ENSG00000119414 | PPP6C | Hs.495128 | 297.5 | PP | 557.55 | PP |
| ENSG00000138073 | PREB | Hs.279784 | 324.3 | PP | 251.45 | PP |
| ENSG00000117569 | PTBP2 | Hs.269895 | 183 | PP | 160.15 | PP |
| ENSG00000185129 | PURA | Hs.443121 | 149.5 | PP | 224.95 | PP |
| ENSG00000138069 | RAB1A | Hs.310645 | 70.8 | PP | 109.75 | PM |
| ENSG00000119318 | RAD23B | Hs.521640 | 156.2 | PP | 211.05 | PP |
| ENSG00000077092 | RARB | Hs.436538 | 111.5 | PP | 156.85 | PP |
| ENSG00000125976 | RBM12 | — | 321.1 | PP | 313.35 | PP |
| ENSG00000153250 | RBMS1 | Hs.470412 | 144.9 | PP | 216.05 | PP |
| ENSG00000067560 | RHOA | Hs.247077 | 1537.7 | PP | 2368.8 | PP |
| ENSG00000137075 | RNF38 | Hs.333503 | 322.6 | PP | 227.7 | PP |
| ENSG00000160633 | SAFB | Hs.23978 | 376.8 | PP | 261.35 | PP |
| ENSG00000138760 | SCARB2 | Hs.349656 | 81.4 | PP | 116.15 | PP |
| ENSG00000161547 | SFRS2 | Hs.73965 | 3723.5 | PP | 4390.4 | PP |
| ENSG00000102038 | SMARCA1 | Hs.152292 | 101.1 | PP | 72.95 | PA |
| ENSG00000180283 | SUMO2 | Hs.380973 | 1013.1 | PP | 783 | PP |
| ENSG00000109332 | UBE2D3 | Hs.472031 | 471.2 | PP | 635.4 | PP |
| ENSG00000100242 | UNC84B | Hs.517622 | 560.7 | PP | 333.25 | PP |
| ENSG00000107372 | ZA20D2 | Hs.406096 | 967.2 | PP | 1262.9 | PP |
| ENSG00000086666 | ZA20D3 | Hs.306329 | 735.1 | PP | 517.75 | PP |

221

| | | | | | | |
|---|---|---|---|---|---|---|
| ENSG00000114770 | ABCC5 | Hs.368563 | 373.4 | PP | 322.1 | PM |
| ENSG00000075624 | ACTB | Hs.520640 | 11983.4 | PP | 11502.85 | PP |
| ENSG00000131503 | ANKHD1 | Hs.434219 | 168 | PP | 163.05 | PP |
| ENSG00000177879 | AP3S1 | Hs.406191 | 647.6 | PP | 1020.05 | PP |
| ENSG00000084234 | APLP2 | Hs.370247 | 1126.7 | PP | 668.55 | PP |
| ENSG00000168374 | ARF4 | Hs.148330 | 129 | PP | 157.5 | PP |
| ENSG00000117713 | ARID1A | Hs.468972 | 477.6 | PP | 444.8 | PP |
| ENSG00000170540 | ARL6IP | Hs.75249 | 155.3 | PP | 376.15 | PP |
| ENSG00000163399 | ATP1A1 | Hs.371889 | 370.4 | PP | 539.1 | PP |
| ENSG00000143153 | ATP1B1 | Hs.291196 | 132.4 | PP | 245.55 | PP |
| ENSG00000119866 | BCL11A | Hs.370549 | 363.1 | PP | 387.8 | PP |
| ENSG00000104765 | BNIP3L | Hs.131226 | 239.7 | PP | 375.45 | PP |
| ENSG00000152242 | C18orf25 | Hs.116486 | 129.7 | PP | 130.6 | PA |
| ENSG00000168958 | C2orf33 | Hs.471528 | 235.3 | PP | 302.3 | PP |
| ENSG00000067955 | CBFB | Hs.460988 | 719.1 | PP | 1032 | PP |
| ENSG00000163660 | CCNL1 | Hs.4859 | 1082.6 | PP | 1052.5 | PP |
| ENSG00000155111 | CDC2L6 | Hs.193251 | 325 | PP | 341.25 | PP |
| ENSG00000111276 | CDKN1B | Hs.238990 | 333.5 | PP | 374.65 | PP |
| ENSG00000072609 | CHFR | Hs.507336 | 247.4 | PP | 253.05 | PP |
| ENSG00000131873 | CHSY1 | Hs.110488 | 404.5 | PP | 490.95 | PP |
| ENSG00000125107 | CNOT1 | Hs.460923 | 658.2 | PP | 662.1 | PP |
| ENSG00000145708 | CRHBP | Hs.115617 | 536.3 | PP | 346.1 | PP |
| ENSG00000108848 | CROP | Hs.130293 | 2166.4 | PP | 1637.55 | PP |
| ENSG00000102974 | CTCF | Hs.368367 | 506.1 | PP | 535.55 | PP |
| ENSG00000168036 | CTNNB1 | Hs.476018 | 343.5 | PP | 364.4 | PP |
| ENSG00000149187 | CUGBP1 | Hs.530727 | 383 | PP | 596.3 | PA |
| ENSG00000160967 | CUTL1 | Hs.438974 | 333.3 | PP | 412.55 | PP |
| ENSG00000165659 | DACH1 | Hs.129452 | 95.1 | PP | 144.8 | PP |
| ENSG00000168209 | DDIT4 | Hs.523012 | 736.8 | PP | 535.85 | PP |
| ENSG00000124487 | DDX3X | Hs.380774 | 769.8 | PP | 1229 | PP |
| ENSG00000109606 | DHX15 | Hs.5683 | 1219.8 | PP | 1620.75 | PP |
| ENSG00000183690 | EFHC2 | Hs.521953 | 162.6 | PP | 209.65 | PP |
| ENSG00000104131 | EIF3S1 | Hs.404056 | 340.4 | PP | 365.05 | PP |
| ENSG00000131503 | EIF4EBP3 /// | — | 285.5 | PP | 200 | PP |
| | MASK-BP3 | | | | | |
| ENSG00000110321 | EIF4G2 | Hs.183684 | 2137 | PP | 2701.9 | PP |
| ENSG00000157557 | ETS2 | Hs.517296 | 188.3 | PP | 297.8 | PP |
| ENSG00000138081 | FBXO11 | Hs.553547 | 155.7 | PP | 259.35 | PP |
| ENSG00000143756 | FBXO28 | Hs.64691 | 106.1 | PP | 70.5 | PA |
| ENSG00000170345 | FOS | Hs.25647 | 1254.3 | PP | 1832.65 | PP |
| ENSG00000165879 | FRAT1 | Hs.126057 | 207.4 | PP | 183.35 | PP |
| ENSG00000065135 | GNAI3 | Hs.73799 | 603.4 | PP | 763.55 | PP |
| ENSG00000091527 | H41 | Hs.518265 | 735.1 | PP | 1129.65 | PP |
| ENSG00000100644 | HIF1A | Hs.509554 | 866.5 | PP | 1037.1 | PP |
| ENSG00000163349 | HIPK1 | Hs.532363 | 347.7 | PP | 488.5 | PP |
| ENSG00000177733 | HNRPA0 | Hs.96996 | 655 | PP | 723.4 | PP |
| ENSG00000170144 | HNRPA3 | Hs.516539 | 375.5 | PP | 558.6 | PP |
| ENSG00000138668 | HNRPD | Hs.480073 | 785.7 | PP | 1067.15 | PP |
| ENSG00000120075 | HOXB5 | Hs.149548 | 322 | PP | 238.15 | PP |
| ENSG00000173744 | HRB | Hs.443507 | 202.6 | PP | 405.7 | PP |
| ENSG00000186480 | INSIG1 | Hs.520819 | 196.9 | PP | 304.7 | PP |
| ENSG00000176407 | KCMF1 | Hs.345694 | 751.2 | PP | 938.3 | PP |
| ENSG00000121774 | KHDRBS1 | Hs.445893 | 1363 | PP | 1395.2 | PP |
| ENSG00000136813 | KIAA0368 | Hs.368255 | 153.6 | PP | 135.95 | PP |
| ENSG00000101367 | MAPRE1 | Hs.472437 | 196.1 | PP | 206.85 | PM |
| ENSG00000168906 | MAT2A | Hs.516157 | 1011.4 | PP | 738.6 | PP |
| ENSG00000038274 | MAT2B | Hs.54642 | 609.4 | PP | 461.9 | PP |
| ENSG00000152601 | MBNL1 | Hs.478000 | 294.4 | PP | 864.5 | PP |
| ENSG00000143995 | MEIS1 | Hs.526754 | 452.1 | PP | 262.65 | PP |
| ENSG00000123562 | MORF4L2 | Hs.326387 | 2189.8 | PP | 2626.1 | PP |
| ENSG00000170873 | MTSS1 | Hs.336994 | 152.7 | PP | 194.6 | PP |
| ENSG00000147140 | NONO | Hs.533282 | 2434.4 | PP | 2382.1 | PP |
| ENSG00000153234 | NR4A2 | Hs.165258 | 124.6 | PP | 242.85 | PP |
| ENSG00000115758 | ODC1 | Hs.467701 | 843.3 | PP | 1634.7 | PP |
| ENSG00000147162 | OGT | Hs.405410 | 165.1 | PP | 183.6 | PP |
| ENSG00000172239 | PAIP1 | Hs.482038 | 127.8 | PP | 157 | PP |
| ENSG00000114054 | PCCB | Hs.63788 | 270.6 | PP | 392.75 | PP |
| ENSG00000114209 | PDCD10 | Hs.478150 | 463.6 | PP | 560.4 | PP |
| ENSG00000118495 | PLAGL1 | Hs.444975 | 187.1 | PP | 357.8 | PP |
| ENSG00000136040 | PLXNC1 | Hs.551522 | 354.6 | PP | 387.1 | PP |
| ENSG00000163806 | PPP1CB | Hs.468018 | 65.7 | PP | 135.05 | PP |
| ENSG00000119414 | PPP6C | Hs.495128 | 297.5 | PP | 557.55 | PP |
| ENSG00000116731 | PRDM2 | Hs.371823 | 329.6 | PP | 175.6 | PP |
| ENSG00000138073 | PREB | Hs.279784 | 324.3 | PP | 251.45 | PP |
| ENSG00000117569 | PTBP2 | Hs.269895 | 183 | PP | 160.15 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000138069 | RAB1A | Hs.310645 | 70.8 | PP | 109.75 | PM |
| ENSG00000153201 | RANBP2 | Hs.199561 | 133.1 | PP | 185.85 | PP |
| ENSG00000153250 | RBMS1 | Hs.470412 | 144.9 | PP | 216.05 | PP |
| ENSG00000067560 | RHOA | Hs.247077 | 1537.7 | PP | 2368.8 | PP |
| ENSG00000063978 | RNF4 | Hs.66394 | 115.8 | PP | 325.65 | PP |
| ENSG00000043093 | RP42 | Hs.104613 | 236.5 | PP | 204.45 | PP |
| ENSG00000163602 | RYBP | Hs.7910 | 398 | PP | 462.8 | PP |
| ENSG00000137575 | SDCBP | Hs.200804 | 934 | PP | 955.25 | PP |
| ENSG00000162105 | SHANK2 | Hs.268726 | 137.1 | PP | 128.55 | PP |
| ENSG00000147454 | SLC25A37 | Hs.122514 | 103 | PP | 86.95 | PA |
| ENSG00000080493 | SLC4A4 | Hs.5462 | 124.5 | PP | 268.4 | PP |
| ENSG00000102038 | SMARCA1 | Hs.152292 | 101.1 | PP | 72.95 | PA |
| ENSG00000167323 | STIM1 | Hs.501735 | 143.2 | PP | 99.7 | PP |
| ENSG00000180283 | SUMO2 | Hs.380973 | 1013.1 | PP | 783 | PP |
| ENSG00000140262 | TCF12 | Hs.511504 | 179.4 | PP | 439.15 | PP |
| ENSG00000071564 | TCF3 | Hs.371282 | 303.8 | PP | 276.8 | PP |
| ENSG00000071564 | TCF3 | Hs.371282 | 322.5 | PP | 443.9 | PM |
| ENSG00000148737 | TCF7L2 | Hs.501080 | 102.3 | PP | 100.85 | PP |
| ENSG00000163659 | TIPARP | Hs.12813 | 567.1 | PP | 1548.4 | PP |
| ENSG00000008952 | TLOC1 | Hs.529591 | 321.8 | PP | 343.45 | PP |
| ENSG00000140391 | TSPAN3 | Hs.5062 | 516.6 | PP | 661.45 | PP |
| ENSG00000131508 | UBE2D2 | Hs.108332 | 400.1 | PP | 512.05 | PP |
| ENSG00000170035 | UBE2E3 | Hs.470804 | 311.5 | PP | 258.25 | PP |
| ENSG00000198833 | UBE2J1 | Hs.163776 | 342.7 | PP | 357.2 | PP |
| ENSG00000100242 | UNC84B | Hs.517622 | 560.7 | PP | 333.25 | PP |
| ENSG00000124164 | VAPB | Hs.182625 | 245 | PP | 213.5 | PP |
| ENSG00000144560 | VGLL4 | Hs.373959 | 299.7 | PP | 175.6 | PP |
| ENSG00000158195 | WASF2 | Hs.469244 | 430.1 | PP | 367.5 | PP |
| ENSG00000085433 | WDR47 | Hs.546347 | 90.4 | PP | 116.6 | PP |
| ENSG00000083896 | YT521 | Hs.175955 | 318.2 | PP | 505.9 | PP |
| ENSG00000170949 | ZNF160 | Hs.467236 | 1056.4 | PP | 1016.85 | PP |
| ENSG00000179456 | ZNF238 | Hs.69997 | 235.7 | PP | 241.8 | PP |

123

There are no predicted targets for this microRNA in either prediction
database

213

| | | | | | | |
|---|---|---|---|---|---|---|
| ENSG00000108848 | CROP | Hs.130293 | 2166.4 | PP | 1637.55 | PP |
| ENSG00000110321 | EIF4G2 | Hs.183684 | 2137 | PP | 2701.9 | PP |
| ENSG00000177733 | HNRPA0 | Hs.96996 | 655 | PP | 723.4 | PP |
| ENSG00000010295 | HOM-TES- | Hs.15243 | 407 | PP | 355.35 | PP |
| | 103 | | | | | |
| ENSG00000055208 | MAP3K7IP2 | Hs.269775 | 219.1 | PP | 142.8 | PP |
| ENSG00000005302 | MSL3L1 | Hs.307924 | 302.5 | PP | 200.1 | PP |
| ENSG00000100836 | PABPN1 | Hs.117176 | 758.2 | PP | 790.85 | PP |
| ENSG00000110851 | PRDM4 | Hs.506655 | 188.6 | PP | 183.55 | PP |
| ENSG00000111540 | RAB5B | Hs.77690 | 234.1 | PP | 216.45 | PP |
| ENSG00000010017 | RANBP9 | Hs.306242 | 381.3 | PP | 489.95 | PP |
| ENSG00000134294 | SLC38A2 | Hs.221847 | 634.3 | PP | 1026.55 | PP |
| ENSG00000080503 | SMARCA2 | Hs.298990 | 264.4 | PP | 201.3 | PP |
| ENSG00000137642 | SORL1 | Hs.368592 | 2264.1 | PP | 1084.65 | PP |
| ENSG00000131508 | UBE2D2 | Hs.108332 | 400.1 | PP | 512.05 | PP |
| ENSG00000179456 | ZNF238 | Hs.69997 | 235.7 | PP | 241.8 | PP |

191

| | | | | | | |
|---|---|---|---|---|---|---|
| ENSG00000116489 | CAPZA1 | Hs.514934 | 766.7 | PP | 1165.8 | PP |
| ENSG00000009307 | CSDE1 | Hs.69855 | 1494.1 | PP | 1637.9 | PP |
| ENSG00000120738 | EGR1 | Hs.326035 | 128.6 | PP | 290.15 | PP |
| ENSG00000092847 | EIF2C1 | Hs.22867 | 292.5 | PP | 296.95 | PP |
| ENSG00000135930 | EIF4E2 | Hs.292026 | 538.3 | PP | 611.25 | PP |
| ENSG00000116478 | HDAC1 | Hs.88556 | 286.1 | PP | 397 | PP |
| ENSG00000114315 | HES1 | Hs.250666 | 232.7 | PP | 169.8 | PP |
| ENSG00000165119 | HNRPK | Hs.449171 | 761 | PP | 1269.35 | PP |
| ENSG00000008083 | JARID2 | Hs.269059 | 1015 | PP | 894 | PP |
| ENSG00000182979 | MTA1 | Hs.525629 | 428.2 | PP | 237.75 | PP |
| ENSG00000164190 | NIPBL | Hs.481927 | 165.1 | PP | 145.15 | PP |
| ENSG00000078618 | NRD1 | Hs.551509 | 874.1 | PP | 639.35 | PP |
| ENSG00000120265 | PCMT1 | Hs.279257 | 488.1 | PP | 649.4 | PP |
| ENSG00000115762 | PLEKHB2 | Hs.469944 | 181.7 | PP | 266 | PP |
| ENSG00000137492 | PRKRIR | Hs.503315 | 281.4 | PP | 549.7 | PP |
| ENSG00000117569 | PTBP2 | Hs.269895 | 183 | PP | 160.15 | PP |
| ENSG00000010017 | RANBP9 | Hs.306242 | 381.3 | PP | 489.95 | PP |
| ENSG00000170881 | RNF139 | Hs.492751 | 392.9 | PP | 435.75 | PP |
| ENSG00000171858 | RPS21 | Hs.190968 | 24441.6 | PP | 21034.85 | PP |
| ENSG00000100784 | RPS6KA5 | Hs.510225 | 152.1 | PP | 132.75 | PP |
| ENSG00000159140 | SON | Hs.517262 | 408.8 | PP | 467.15 | PP |
| ENSG00000132388 | UBE2G1 | Hs.462035 | 272.5 | PP | 373.45 | PP |
| ENSG00000165280 | VCP | Hs.529782 | 278.5 | PP | 240.85 | PP |
| ENSG00000115935 | WASPIP | Hs.128067 | 418.1 | PP | 364.25 | PP |

30d

| | | | | | | |
|---|---|---|---|---|---|---|
| ENSG00000114770 | ABCC5 | Hs.368563 | 373.4 | PP | 322.1 | PM |
| ENSG00000136518 | ACTL6A | Hs.435326 | 84.2 | PP | 105.6 | PP |
| ENSG00000177879 | AP3S1 | Hs.406191 | 647.6 | PP | 1020.05 | PP |
| ENSG00000144848 | APG3L | Hs.477126 | 235.6 | PP | 245.05 | PP |
| ENSG00000129675 | ARHGEF6 | Hs.522795 | 1207.5 | PP | 815.1 | PP |
| ENSG00000117713 | ARID1A | Hs.468972 | 477.6 | PP | 444.8 | PP |
| ENSG00000119866 | BCL11A | Hs.370549 | 363.1 | PP | 387.8 | PP |
| ENSG00000110075 | C11orf23 | Hs.503022 | 402.6 | PP | 319.5 | PA |
| ENSG00000139746 | C13orf10 | Hs.131715 | 119.8 | PP | 178.7 | PP |
| ENSG00000101084 | C20orf24 | Hs.184062 | 878.9 | PP | 1348.2 | PP |
| ENSG00000127022 | CANX | Hs.529890 | 1003.1 | PP | 2097.75 | PP |
| ENSG00000100261 | CBX6 | Hs.511952 | 308 | PP | 255.7 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000070831 | CDC42 | Hs.467637 | 620.1 | PP | 437.75 | PP |
| ENSG00000153922 | CHD1 | Hs.519474 | 89.4 | PP | 202.95 | PP |
| ENSG00000111596 | CNOT2 | Hs.133350 | 431.4 | PP | 399.2 | PP |
| ENSG00000099942 | CRKL | Hs.5613 | 126.4 | PP | 192.1 | PP |
| ENSG00000009307 | CSDE1 | Hs.69855 | 1494.1 | PP | 1637.9 | PP |
| ENSG00000103653 | CSK | Hs.77793 | 348.1 | PP | 217.35 | PA |
| ENSG00000113712 | CSNK1A1 | Hs.442592 | 1045.3 | PP | 1263.25 | PP |
| ENSG00000168209 | DDIT4 | Hs.523012 | 736.8 | PP | 535.85 | PP |
| ENSG00000142634 | EFHD2 | Hs.465374 | 292.3 | PP | 242.65 | PA |
| ENSG00000092847 | EIF2C1 | Hs.22867 | 292.5 | PP | 296.95 | PP |
| ENSG00000120533 | ENY2 | Hs.492555 | 316.2 | PP | 375.85 | PP |
| ENSG00000120616 | EPC1 | Hs.167805 | 148.9 | PP | 88.7 | PA |
| ENSG00000157554 | ERG | Hs.473819 | 369.4 | PP | 298.05 | PP |
| ENSG00000120705 | ETF1 | Hs.483494 | 253.8 | PP | 461.55 | PP |
| ENSG00000182944 | EWSR1 | Hs.374477 | 389.7 | PP | 457.25 | PP |
| ENSG00000150907 | FOXO1A | Hs.370666 | 253.6 | PP | 140.95 | PP |
| ENSG00000145907 | G3BP | Hs.3353 | 167.7 | PP | 319.25 | PP |
| ENSG00000141429 | GALNT1 | Hs.514806 | 220 | PP | 273.8 | PP |
| ENSG00000109586 | GALNT7 | Hs.127407 | 97.2 | PP | 153.95 | PP |
| ENSG00000001084 | GCLC | Hs.271264 | 227.6 | PP | 219.2 | PP |
| ENSG00000177733 | HNRPA0 | Hs.96996 | 655 | PP | 723.4 | PP |
| ENSG00000170144 | HNRPA3 | Hs.516539 | 375.5 | PP | 558.6 | PP |
| ENSG00000152795 | HNRPDL | Hs.527105 | 1931.8 | PP | 1802.05 | PP |
| ENSG00000099783 | HNRPM | Hs.465808 | 968.5 | PP | 880.2 | PP |
| ENSG00000044574 | HSPA5 | Hs.522394 | 1536.9 | PP | 3296.1 | PP |
| ENSG00000115548 | JMJD1A | Hs.531819 | 264 | PP | 207.05 | PP |
| ENSG00000102753 | KPNA3 | Hs.527919 | 250.8 | PP | 391.35 | PP |
| ENSG00000143815 | LBR | Hs.435166 | 1166 | PP | 1807.35 | PP |
| ENSG00000065548 | LEREPO4 | Hs.368598 | 265.5 | PP | 402.5 | PP |
| ENSG00000111885 | MAN1A1 | Hs.102788 | 309.1 | PP | 151 | PP |
| ENSG00000166974 | MAPRE2 | Hs.532824 | 857.4 | PP | 715.5 | PP |
| ENSG00000168906 | MAT2A | Hs.516157 | 1011.4 | PP | 738.6 | PP |
| ENSG00000182979 | MTA1 | Hs.525629 | 428.2 | PP | 237.75 | PP |
| ENSG00000147649 | MTDH | Hs.377155 | 258.6 | PP | 329.8 | PP |
| ENSG00000005810 | MYCBP2 | Hs.151411 | 187 | PP | 643.55 | PP |
| ENSG00000120837 | NFYB | Hs.84928 | 204.1 | PP | 272.85 | PP |
| ENSG00000153234 | NR4A2 | Hs.165258 | 124.6 | PP | 242.85 | PP |
| ENSG00000177425 | PAWR | Hs.406074 | 148.3 | PP | 133.85 | PP |
| ENSG00000105835 | PBEF1 | Hs.489615 | 96.8 | PP | 138.65 | PP |
| ENSG00000136040 | PLXNC1 | Hs.551522 | 354.6 | PP | 387.1 | PP |
| ENSG00000112245 | PTP4A1 | Hs.227777 | 439.5 | PP | 431.2 | PP |
| ENSG00000175354 | PTPN2 | Hs.123352 | 759.9 | PP | 273 | PP |
| ENSG00000130508 | PXDN | Hs.332197 | 280.3 | PP | 114.7 | PA |
| ENSG00000048991 | R3HDM | Hs.412462 | 217.6 | PP | 376.75 | PP |
| ENSG00000103769 | RAB11A | Hs.321541 | 1087.7 | PP | 1020.15 | PP |
| ENSG00000138069 | RAB1A | Hs.310645 | 70.8 | PP | 109.75 | PM |
| ENSG00000119318 | RAD23B | Hs.521640 | 156.2 | PP | 211.05 | PP |
| ENSG00000145715 | RASA1 | Hs.553501 | 138.8 | PP | 103.6 | PP |
| ENSG00000125976 | RBM12 | — | 321.1 | PP | 313.35 | PP |
| ENSG00000106615 | RHEB | Hs.283521 | 943.5 | PP | 1172.5 | PP |
| ENSG00000144580 | RQCD1 | Hs.148767 | 284.7 | PP | 361.25 | PP |
| ENSG00000122545 | 7-Sep | Hs.191346 | 815 | PP | 1146.5 | PP |
| ENSG00000115524 | SF3B1 | Hs.471011 | 2127.6 | PP | 2173.9 | PP |
| ENSG00000115875 | SFRS7 | Hs.309090 | 378.7 | PP | 743.5 | PP |
| ENSG00000113558 | SKP1A | Hs.171626 | 657.2 | PP | 969.15 | PP |
| ENSG00000059804 | SLC2A3 | Hs.419240 | 275.9 | PP | 351.5 | PP |
| ENSG00000112305 | SMAP1 | Hs.485717 | 205.2 | PP | 129.15 | PP |
| ENSG00000077147 | SMBP | Hs.500674 | 358.3 | PP | 250.05 | PP |
| ENSG00000159140 | SON | Hs.517262 | 408.8 | PP | 467.15 | PP |
| ENSG00000124766 | SOX4 | Hs.357901 | 2351.7 | PP | 2500.65 | PP |
| ENSG00000180283 | SUMO2 | Hs.380973 | 1013.1 | PP | 783 | PP |
| ENSG00000008282 | SYPL1 | Hs.80919 | 157.3 | PP | 190.55 | PP |
| ENSG00000136205 | TENS1 | Hs.520814 | 443.4 | PP | 221.15 | PP |
| ENSG00000144043 | TEX261 | Hs.516087 | 360.9 | PP | 229.45 | PA |
| ENSG00000114126 | TFDP2 | Hs.379018 | 253.2 | PP | 196.3 | PA |
| ENSG00000116001 | TIA1 | Hs.516075 | 151.6 | PP | 145 | PP |
| ENSG00000170348 | TMP21 | Hs.74137 | 293.5 | PP | 280.3 | PA |
| ENSG00000063244 | U2AF2 | Hs.528007 | 191 | PP | 227.15 | PP |
| ENSG00000131508 | UBE2D2 | Hs.108332 | 400.1 | PP | 512.05 | PP |
| ENSG00000103275 | UBE2I | Hs.302903 | 452.9 | PP | 427.25 | PP |
| ENSG00000009335 | UBE3C | Hs.118351 | 67.5 | PP | 175.9 | PM |
| ENSG00000118900 | UBN1 | Hs.440219 | 138.4 | PP | 302.15 | PP |
| ENSG00000162607 | USP1 | Hs.35086 | 275.3 | PP | 614.05 | PP |
| ENSG00000101558 | VAPA | Hs.165195 | 1051.8 | PP | 1198.45 | PP |
| ENSG00000108828 | VAT1 | Hs.514199 | 649.7 | PP | 585.4 | PP |
| ENSG00000165280 | VCP | Hs.529782 | 278.5 | PP | 240.85 | PP |
| ENSG00000107372 | ZA20D2 | Hs.406096 | 967.2 | PP | 1262.9 | PP |
| ENSG00000152518 | ZFP36L2 | Hs.503093 | 4553.7 | PP | 5205.6 | PP |
| ENSG00000179456 | ZNF238 | Hs.69997 | 235.7 | PP | 241.8 | PP |

25

| | | | | | | |
|---|---|---|---|---|---|---|
| ENSG00000138031 | ADCY3 | Hs.467898 | 208.6 | PP | 178.4 | PP |
| ENSG00000117713 | ARID1A | Hs.468972 | 477.6 | PP | 444.8 | PP |
| ENSG00000143153 | ATP1B1 | Hs.291196 | 132.4 | PP | 245.55 | PP |
| ENSG00000174437 | ATP2A2 | Hs.506759 | 203.5 | PP | 255.4 | PP |
| ENSG00000155096 | AZIN1 | Hs.459106 | 551.2 | PP | 619.2 | PP |
| ENSG00000159388 | BTG2 | Hs.519162 | 588.2 | PP | 538.3 | PP |
| ENSG00000112237 | CCNC | Hs.430646 | 273.3 | PP | 380.8 | PP |
| ENSG00000118260 | CREB1 | Hs.516646 | 122.9 | PP | 230.35 | PP |
| ENSG00000102974 | CTCF | Hs.368367 | 506.1 | PP | 535.55 | PP |
| ENSG00000132153 | DHX30 | Hs.517948 | 209.5 | PP | 193.5 | PP |
| ENSG00000128590 | DNAJB9 | Hs.6790 | 211.5 | PP | 433.55 | PP |
| ENSG00000112242 | E2F3 | Hs.269408 | 295.6 | PP | 585.95 | PP |
| ENSG00000123908 | EIF2C2 | Hs.449415 | 150.2 | PP | 115.85 | PP |
| ENSG00000104131 | EIF3S1 | Hs.404056 | 340.4 | PP | 365.05 | PP |
| ENSG00000100664 | EIF5 | Hs.433702 | 96.4 | PP | 55.8 | PA |
| ENSG00000109920 | FNBP4 | Hs.6834 | 501.2 | PP | 497.2 | PP |
| ENSG00000114416 | FXR1 | Hs.478407 | 143.9 | PP | 155.75 | PP |
| ENSG00000057608 | GDI2 | Hs.299055 | 198.2 | PP | 260.65 | PP |
| ENSG00000172534 | HCFC1 | Hs.83634 | 146.2 | PP | 233.9 | PA |
| ENSG00000122566 | HNRPA2B1 | Hs.487774 | 1802.2 | PP | 2268.3 | PP |
| ENSG00000170144 | HNRPA3 | Hs.516539 | 375.5 | PP | 558.6 | PP |
| ENSG00000153807 | HOXA10 | Hs.110637 | 161.3 | PP | 200.7 | PA |
| ENSG00000173744 | HRB | Hs.443507 | 202.6 | PP | 405.7 | PP |
| ENSG00000144381 | HSPD1 | Hs.113684 | 2868.6 | PP | 5689.3 | PP |
| ENSG00000143164 | IQWD1 | Hs.435741 | 168.5 | PP | 156.4 | PP |
| ENSG00000102753 | KPNA3 | Hs.527919 | 250.8 | PP | 391.35 | PA |
| ENSG00000012983 | MAP4K5 | Hs.130491 | 66 | PP | 116 | PP |
| ENSG00000112118 | MCM3 | Hs.179565 | 509.1 | PP | 637.3 | PP |
| ENSG00000081189 | MEF2C | Hs.444409 | 426 | PP | 437.85 | PP |
| ENSG00000165943 | MOAP1 | Hs.24719 | 527.1 | PP | 299.1 | PP |
| ENSG00000066136 | NFYC | Hs.233458 | 115.5 | PP | 168 | PP |
| ENSG00000170515 | PA2G4 | Hs.478044 | 251.3 | PP | 523.75 | PP |
| ENSG00000157654 | PALM2- | Hs.259461 | 181.4 | PP | 163.75 | PP |
| | AKAP2 | | | | | |
| ENSG00000145730 | PAM | Hs.369430 | 187.3 | PP | 178.45 | PP |
| ENSG00000169564 | PCBP1 | Hs.2853 | 1587.4 | PP | 1289.35 | PP |
| ENSG00000102225 | PCTK1 | Hs.496068 | 135 | PP | 88.45 | PA |
| ENSG00000059758 | PCTK2 | Hs.506415 | 118.3 | PP | 220.9 | PP |
| ENSG00000112511 | PHF1 | Hs.166204 | 903.7 | PP | 604.1 | PP |
| ENSG00000112941 | POLS | Hs.481542 | 355.5 | PP | 367.35 | PP |
| ENSG00000138430 | PTD004 | Hs.157351 | 848.2 | PP | 1052.6 | PP |
| ENSG00000137075 | RNF38 | Hs.333503 | 322.6 | PP | 227.7 | PP |
| ENSG00000063978 | RNF4 | Hs.66394 | 115.8 | PP | 325.65 | PP |
| ENSG00000156642 | SDFR1 | Hs.187866 | 169.4 | PP | 193.05 | PP |
| ENSG00000134294 | SLC38A2 | Hs.221847 | 634.3 | PP | 1026.55 | PP |
| ENSG00000184602 | SNN | Hs.459952 | 234.4 | PP | 239.3 | PP |
| ENSG00000120833 | SOCS2 | Hs.485572 | 715.8 | PP | 806.65 | PP |
| ENSG00000137642 | SORL1 | Hs.368592 | 2264.1 | PP | 1084.65 | PP |
| ENSG00000124766 | SOX4 | Hs.357901 | 2351.7 | PP | 2500.65 | PP |
| ENSG00000101972 | STAG2 | Hs.496710 | 392 | PP | 375.2 | PP |
| ENSG00000165169 | TCTE1L | Hs.446392 | 323 | PP | 369.75 | PP |
| ENSG00000177426 | TGIF | Hs.373550 | 116.7 | PP | 95.35 | PM |
| ENSG00000165699 | TSC1 | Hs.370854 | 232.7 | PP | 242.3 | PP |
| ENSG00000169764 | UGP2 | Hs.516217 | 656.9 | PP | 560.95 | PP |
| ENSG00000135655 | USP15 | Hs.434951 | 197.3 | PP | 160.35 | PP |
| ENSG00000143952 | VPS54 | Hs.48499 | 187.9 | PP | 151.5 | PP |
| ENSG00000166913 | YWHAB | Hs.279920 | 222.4 | PP | 288.75 | PP |
| ENSG00000170949 | ZNF160 | Hs.467236 | 1056.4 | PP | 1016.85 | PP |

130a

| | | | | | | |
|---|---|---|---|---|---|---|
| ENSG00000165029 | ABCA1 | Hs.429294 | 175.1 | PP | 156.5 | PP |
| ENSG00000114770 | ABCC5 | Hs.368563 | 373.4 | PP | 322.1 | PM |
| ENSG00000151726 | ACSL1 | Hs.406678 | 180.6 | PP | 193.15 | PP |
| ENSG00000023516 | AKAP11 | Hs.105105 | 88.5 | PP | 69.7 | PP |
| ENSG00000170017 | ALCAM | Hs.150693 | 528.9 | PP | 695.45 | PP |
| ENSG00000137962 | ARHGAP29 | Hs.483238 | 72.2 | PP | 65.3 | PP |
| ENSG00000117713 | ARID1A | Hs.468972 | 477.6 | PP | 444.8 | PP |
| ENSG00000054267 | ARID4B | Hs.533633 | 227 | PP | 252.45 | PP |
| ENSG00000134108 | ARL10C | Hs.250009 | 578.1 | PP | 591.95 | PP |
| ENSG00000128989 | ARPP-19 | Hs.512908 | 247.7 | PP | 139.7 | PP |
| ENSG00000143970 | ASXL2 | Hs.119815 | 694.8 | PP | 535.25 | PP |
| ENSG00000174437 | ATP2A2 | Hs.506759 | 203.5 | PP | 255.4 | PP |
| ENSG00000017260 | ATP2C1 | Hs.546361 | 229.1 | PP | 322.9 | PP |
| ENSG00000147416 | ATP6V1B2 | Hs.295917 | 179.5 | PP | 183.85 | PP |
| ENSG00000198604 | BAZ1A | Hs.509140 | 102.4 | PP | 111.2 | PA |
| ENSG00000119866 | BCL11A | Hs.370549 | 363.1 | PP | 387.8 | PP |
| ENSG00000029363 | BCLAF1 | Hs.486542 | 659.8 | PP | 1252.25 | PP |
| ENSG00000095564 | BTAF1 | Hs.500526 | 220.4 | PP | 333.3 | PP |
| ENSG00000133639 | BTG1 | Hs.255935 | 955.9 | PP | 1057.95 | PP |
| ENSG00000154640 | BTG3 | Hs.473420 | 315.6 | PP | 378.5 | PP |
| ENSG00000119906 | C10orf6 | Hs.447458 | 154.8 | PP | 126.8 | PP |
| ENSG00000110075 | C11orf23 | Hs.503022 | 402.6 | PP | 319.5 | PA |
| ENSG00000134153 | C15orf24 | Hs.160565 | 612.5 | PP | 706.2 | PP |
| ENSG00000152242 | C18orf25 | Hs.116486 | 129.7 | PP | 130.6 | PA |
| ENSG00000101166 | C20orf45 | Hs.3945 | 93.1 | PP | 74.8 | PP |
| ENSG00000162959 | C2orf4 | Hs.444969 | 410.5 | PP | 397.7 | PA |
| ENSG00000031003 | C5orf5 | Hs.483473 | 256.3 | PP | 316.85 | PP |
| ENSG00000112308 | C6orf62 | Hs.519930 | 330.6 | PP | 509.45 | PP |
| ENSG00000112308 | C6orf62 | Hs.519930 | 566.8 | PP | 770.5 | PP |
| ENSG00000112576 | CCND3 | Hs.534307 | 539.8 | PP | 402.15 | PP |
| ENSG00000110848 | CD69 | Hs.208854 | 1631.5 | PP | 1082.1 | PP |
| ENSG00000155111 | CDC2L6 | Hs.193251 | 325 | PP | 341.25 | PP |
| ENSG00000143126 | CELSR2 | Hs.57652 | 215.4 | PP | 148.65 | PP |
| ENSG00000177200 | CHD9 | Hs.530936 | 222.6 | PP | 263 | PP |
| ENSG00000131873 | CHSY1 | Hs.110488 | 404.5 | PP | 490.95 | PP |
| ENSG00000141367 | CLTC | Hs.491351 | 1169 | PP | 1399.6 | PP |
| ENSG00000162368 | CMPK | Hs.11463 | 333.8 | PP | 336.55 | PP |
| ENSG00000100528 | CNIH | Hs.294603 | 252.6 | PP | 391.7 | PP |
| ENSG00000198791 | CNOT7 | Hs.170553 | 250.3 | PP | 508.5 | PP |
| ENSG00000135940 | COX5B | Hs.1342 | 645 | PP | 568.3 | PA |
| ENSG00000115944 | COX7A2L | Hs.339639 | 1251.1 | PP | 1258.85 | PP |
| ENSG00000005339 | CREBBP | Hs.459759 | 287.2 | PP | 239.6 | PP |
| ENSG00000108848 | CROP | Hs.130293 | 2166.4 | PP | 1637.55 | PP |
| ENSG00000009307 | CSDE1 | Hs.69855 | 1494.1 | PP | 1637.9 | PP |
| ENSG00000103653 | CSK | Hs.77793 | 348.1 | PP | 217.35 | PA |
| ENSG00000113712 | CSNK1A1 | Hs.442592 | 1045.3 | PP | 1263.25 | PP |
| ENSG00000168036 | CTNNB1 | Hs.476018 | 343.5 | PP | 364.4 | PP |
| ENSG00000036257 | CUL3 | Hs.372286 | 554.5 | PP | 473.25 | PP |
| ENSG00000166266 | CUL5 | Hs.440320 | 285.8 | PP | 246.8 | PP |
| ENSG00000083799 | CYLD | Hs.432993 | 137.6 | PP | 148.8 | PP |
| ENSG00000124487 | DDX3X | Hs.380774 | 769.8 | PP | 1229 | PP |
| ENSG00000124795 | DEK | Hs.484813 | 901.9 | PP | 1127.65 | PP |
| ENSG00000109606 | DHX15 | Hs.5683 | 1219.8 | PP | 1620.75 | PP |
| ENSG00000132002 | DNAJB1 | Hs.515210 | 453.4 | PP | 2770.05 | PP |
| ENSG00000105993 | DNAJB6 | Hs.490745 | 189.7 | PP | 348.5 | PP |
| ENSG00000092964 | DPYSL2 | Hs.173381 | 505.1 | PP | 495.1 | PP |
| ENSG00000117505 | DR1 | Hs.348418 | 117.2 | PP | 188.65 | PP |
| ENSG00000092847 | EIF2C1 | Hs.22867 | 292.5 | PP | 296.95 | PP |
| ENSG00000151247 | EIF4E | Hs.249718 | 156.7 | PP | 224 | PP |
| ENSG00000111145 | ELK3 | Hs.46523 | 372.1 | PP | 280.75 | PP |
| ENSG00000012660 | ELOVL5 | Hs.520189 | 681.6 | PP | 667.3 | PP |
| ENSG00000120616 | EPC1 | Hs.167805 | 148.9 | PP | 88.7 | PA |
| ENSG00000124882 | EREG | Hs.115263 | 351.8 | PP | 535.15 | PP |
| ENSG00000124491 | F13A1 | Hs.335513 | 290.7 | PP | 299.8 | PP |
| ENSG00000119979 | FAM45B /// | Hs.434241 | 554.8 | PP | 690.2 | PP |
| | FAM45A | | | | | |
| ENSG00000139146 | FAM60A | Hs.505154 | 598.1 | PP | 788 | PP |
| ENSG00000166147 | FBN1 | Hs.146447 | 195.2 | PP | 178.7 | PP |
| ENSG00000173120 | FBXL11 | Hs.124147 | 237 | PP | 192.5 | PP |
| ENSG00000118564 | FBXL5 | Hs.479208 | 410.6 | PP | 319.15 | PP |
| ENSG00000138081 | FBXO11 | Hs.553547 | 155.7 | PP | 259.35 | PP |
| ENSG00000143756 | FBXO28 | Hs.64691 | 106.1 | PP | 70.5 | PA |
| ENSG00000134884 | FLJ10154 | Hs.508644 | 843.1 | PP | 1079.5 | PP |
| ENSG00000153561 | FLJ13910 | Hs.75277 | 194.1 | PP | 143.15 | PP |
| ENSG00000132589 | FLOT2 | Hs.514038 | 393.4 | PP | 355.45 | PP |
| ENSG00000138757 | G3BP2 | Hs.303676 | 242.8 | PP | 428.55 | PP |
| ENSG00000116717 | GADD45A | Hs.80409 | 266 | PP | 245.7 | PP |
| ENSG00000127955 | GNAI1 | Hs.134587 | 104.9 | PP | 139.15 | PP |
| ENSG00000135677 | GNS | Hs.334534 | 261.1 | PP | 248.15 | PP |
| ENSG00000106070 | GRB10 | Hs.164060 | 550.7 | PP | 574.15 | PP |
| ENSG00000112406 | HECA | Hs.197644 | 352.2 | PP | 377.45 | PP |
| ENSG00000164683 | HEY1 | Hs.234434 | 164.1 | PP | 151.85 | PP |
| ENSG00000010818 | HIVEP2 | Hs.510172 | 149.6 | PP | 157.9 | PP |
| ENSG00000169045 | HNRPH1 | Hs.202166 | 1986.4 | PP | 2132.6 | PP |
| ENSG00000126945 | HNRPH2 | Hs.278857 | 189.2 | PP | 158.45 | PP |
| ENSG00000099783 | HNRPM | Hs.465808 | 968.5 | PP | 880.2 | PP |
| ENSG00000105323 | HNRPUL1 | Hs.155218 | 406.9 | PP | 401.15 | PP |
| ENSG00000106004 | HOXA5 | Hs.37034 | 507.2 | PP | 499.3 | PP |
| ENSG00000173744 | HRB | Hs.443507 | 202.6 | PP | 405.7 | PP |
| ENSG00000164164 | HSHIN1 | Hs.270851 | 303.1 | PP | 312.65 | PP |
| ENSG00000067829 | IDH3G | Hs.410197 | 249.2 | PP | 156.95 | PA |
| ENSG00000145703 | IQGAP2 | Hs.291030 | 537.8 | PP | 457.9 | PP |
| ENSG00000125347 | IRF1 | Hs.436061 | 467.7 | PP | 299.6 | PP |
| ENSG00000170852 | KBTBD2 | Hs.372541 | 412.2 | PP | 381.65 | PP |
| ENSG00000131149 | KIAA0182 | Hs.461647 | 273.2 | PP | 316.15 | PP |
| ENSG00000143702 | KIAA0470 | Hs.533635 | 146.2 | PP | 325 | PP |
| ENSG00000126775 | KIAA0831 | Hs.414809 | 254.4 | PP | 217.45 | PP |
| ENSG00000105063 | KIAA1115 | Hs.515610 | 227.5 | PP | 221.6 | PM |
| ENSG00000157404 | KIT | Hs.479754 | 339 | PP | 355.6 | PP |
| ENSG00000136826 | KLF4 | Hs.376206 | 536.5 | PP | 1193.7 | PP |
| ENSG00000114030 | KPNA1 | Hs.161008 | 121.8 | PP | 91.95 | PP |
| ENSG00000102753 | KPNA3 | Hs.527919 | 250.8 | PP | 391.35 | PP |
| ENSG00000068697 | LAPTM4A | Hs.467807 | 1059.2 | PP | 1145.5 | PP |
| ENSG00000107929 | LARP5 | Hs.498455 | 274.8 | PP | 203.9 | PP |
| ENSG00000123684 | LPGAT1 | Hs.497674 | 106 | PP | 114.3 | PP |
| ENSG00000135387 | M11S1 | Hs.471818 | 464.9 | PP | 451.45 | PP |
| ENSG00000003056 | M6PR | Hs.134084 | 566.2 | PP | 575.55 | PP |
| ENSG00000121579 | MAK3 | Hs.269528 | 268.8 | PP | 307 | PP |
| ENSG00000161021 | MAML1 | Hs.484349 | 331.3 | PP | 280.35 | PP |
| ENSG00000197442 | MAP3K5 | Hs.186486 | 227.6 | PP | 272.25 | PP |
| ENSG00000047849 | MAP4 | Hs.517949 | 302.6 | PP | 276.5 | PP |
| ENSG00000135525 | MAP7 | Hs.486548 | 325.7 | PP | 390.1 | PP |
| ENSG00000101367 | MAPRE1 | Hs.472437 | 196.1 | PP | 206.85 | PM |
| ENSG00000075413 | MARK3 | Hs.35828 | 129.9 | PP | 75.05 | PP |
| ENSG00000038274 | MAT2B | Hs.54642 | 609.4 | PP | 461.9 | PP |
| ENSG00000152601 | MBNL1 | Hs.478000 | 294.4 | PP | 864.5 | PP |
| ENSG00000138092 | MGC11266 | Hs.546428 | 152.2 | PP | 90.95 | PA |
| ENSG00000133858 | MGC23401 | Hs.527874 | 291 | PP | 309.55 | PP |
| ENSG00000133606 | MKRN1 | Hs.490347 | 392.7 | PP | 350.15 | PP |
| ENSG00000051825 | MPHOSPH9 | Hs.507175 | 261.2 | PP | 160.5 | PP |
| ENSG00000108389 | MTMR4 | Hs.514373 | 330.7 | PP | 407.2 | PP |
| ENSG00000158747 | NBL1 | Hs.439671 | 370.3 | PP | 360.6 | PA |
| ENSG00000166579 | NDEL1 | Hs.372123 | 218.3 | PP | 187.2 | PP |
| ENSG00000082641 | NFE2L1 | Hs.514284 | 735.5 | PP | 726.35 | PP |
| ENSG00000140157 | NIPA2 | Hs.370367 | 284.1 | PP | 685.9 | PP |
| ENSG00000164190 | NIPBL | Hs.481927 | 165.1 | PP | 145.15 | PP |
| ENSG00000149308 | NPAT | Hs.171061 | 171.1 | PP | 140.65 | PP |
| ENSG00000124789 | NUP153 | Hs.121088 | 216.4 | PP | 291.8 | PP |
| ENSG00000147162 | OGT | Hs.405410 | 165.1 | PP | 183.6 | PP |
| ENSG00000141447 | OSBPL1A | Hs.370725 | 204.6 | PP | 211.75 | PP |
| ENSG00000090060 | PAPOLA | Hs.253726 | 307.4 | PP | 286 | PP |
| ENSG00000059758 | PCTK2 | Hs.506415 | 118.3 | PP | 220.9 | PP |
| ENSG00000145431 | PDGFC | Hs.148162 | 473.1 | PP | 395.4 | PP |
| ENSG00000197329 | PELI1 | Hs.7886 | 83.3 | PP | 154.5 | PP |
| ENSG00000025293 | PHF20 | Hs.517044 | 249.8 | PP | 173.65 | PP |
| ENSG00000118482 | PHF3 | Hs.348921 | 260 | PP | 268.9 | PP |
| ENSG00000051382 | PIK3CB | Hs.239818 | 275.1 | PP | 280.95 | PP |
| ENSG00000163806 | PPP1CB | Hs.468018 | 65.7 | PP | 135.05 | PP |
| ENSG00000113575 | PPP2CA | Hs.483408 | 360 | PP | 370.4 | PP |
| ENSG00000115825 | PRKD3 | Hs.173536 | 97.5 | PP | 111.1 | PP |
| ENSG00000197746 | PSAP | Hs.523004 | 1015.2 | PP | 647.25 | PP |
| ENSG00000117569 | PTBP2 | Hs.269895 | 183 | PP | 160.15 | PP |
| ENSG00000120899 | PTK2B | Hs.491322 | 333.1 | PP | 183.65 | PP |
| ENSG00000112245 | PTP4A1 | Hs.227777 | 439.5 | PP | 431.2 | PP |
| ENSG00000175354 | PTPN2 | Hs.123352 | 759.9 | PP | 273 | PP |
| ENSG00000185129 | PURA | Hs.443121 | 149.5 | PP | 224.95 | PP |
| ENSG00000130508 | PXDN | Hs.332197 | 280.3 | PP | 114.7 | PA |
| ENSG00000112531 | QKI | Hs.510324 | 284.1 | PP | 564.45 | PP |
| ENSG00000048991 | R3HDM | Hs.412462 | 217.6 | PP | 376.75 | PP |
| ENSG00000138069 | RAB1A | Hs.310645 | 70.8 | PP | 109.75 | PM |
| ENSG00000144566 | RAB5A | Hs.475663 | 471.3 | PP | 578.35 | PP |
| ENSG00000111540 | RAB5B | Hs.77690 | 234.1 | PP | 216.45 | PP |
| ENSG00000167461 | RAB8A | Hs.5947 | 675.2 | PP | 756.75 | PP |
| ENSG00000119318 | RAD23B | Hs.521640 | 156.2 | PP | 211.05 | PP |
| ENSG00000017797 | RALBP1 | Hs.528993 | 379 | PP | 244.05 | PP |
| ENSG00000077092 | RARB | Hs.436538 | 111.5 | PP | 156.85 | PP |
| ENSG00000067560 | RHOA | Hs.247077 | 1537.7 | PP | 2368.8 | PP |
| ENSG00000022840 | RNF10 | Hs.442798 | 327.5 | PP | 282.35 | PM |
| ENSG00000123091 | RNF11 | Hs.309641 | 125.3 | PP | 161.5 | PP |
| ENSG00000137075 | RNF38 | Hs.333503 | 322.6 | PP | 227.7 | PP |
| ENSG00000143947 | RPS27A | Hs.311640 | 7964.6 | PP | 6951 | PP |
| ENSG00000100784 | RPS6KA5 | Hs.510225 | 152.1 | PP | 132.75 | PP |
| ENSG00000130779 | RSN | Hs.524809 | 76.6 | PP | 58.05 | PP |
| ENSG00000115310 | RTN4 | Hs.429581 | 710.8 | PP | 1341.5 | PP |
| ENSG00000020633 | RUNX3 | Hs.170019 | 541.5 | PP | 272.8 | PP |
| ENSG00000163602 | RYBP | Hs.7910 | 398 | PP | 462.8 | PP |
| ENSG00000198838 | RYR3 | Hs.369250 | 116.8 | PP | 131.8 | PP |
| ENSG00000182568 | SATB1 | Hs.517717 | 829.9 | PP | 652.05 | PP |
| ENSG00000140497 | SCAMP2 | Hs.458917 | 411.8 | PP | 495 | PP |
| ENSG00000156642 | SDFR1 | Hs.187866 | 169.4 | PP | 193.05 | PP |
| ENSG00000122545 | 7-Sep | Hs.191346 | 815 | PP | 1146.5 | PP |
| ENSG00000179833 | SERTAD2 | Hs.77293 | 498.4 | PP | 556.2 | PP |
| ENSG00000116754 | SFRS11 | Hs.479693 | 819.9 | PP | 1077.55 | PP |
| ENSG00000162105 | SHANK2 | Hs.268726 | 137.1 | PP | 128.55 | PP |
| ENSG00000112305 | SMAP1 | Hs.485717 | 205.2 | PP | 129.15 | PP |
| ENSG00000102172 | SMS | Hs.529887 | 287.8 | PP | 275.25 | PP |
| ENSG00000163788 | SNRK | Hs.476052 | 320.6 | PP | 436.65 | PP |
| ENSG00000089006 | SNX5 | Hs.316890 | 327.9 | PP | 403.25 | PP |
| ENSG00000159140 | SON | Hs.517262 | 408.8 | PP | 467.15 | PP |
| ENSG00000115904 | SOS1 | Hs.278733 | 144 | PP | 169.4 | PP |
| ENSG00000124766 | SOX4 | Hs.357901 | 2351.7 | PP | 2500.65 | PP |
| ENSG00000065526 | SPEN | Hs.270499 | 257.6 | PP | 262.7 | PP |
| ENSG00000115306 | SPTBN1 | Hs.503178 | 589.9 | PP | 344.65 | PP |
| ENSG00000124783 | SSR1 | Hs.114033 | 600.7 | PP | 686.65 | PP |
| ENSG00000112079 | STK38 | Hs.409578 | 365.8 | PP | 304.4 | PP |
| ENSG00000173812 | SUI1 | Hs.150580 | 6046 | PP | 6098.85 | PP |
| ENSG00000101945 | SUV39H1 | Hs.522639 | 267.8 | PP | 383.35 | PP |
| ENSG00000178691 | SUZ12 | Hs.462732 | 602.2 | PP | 633.3 | PP |
| ENSG00000135090 | TAOK3 | — | 518.8 | PP | 394 | PP |
| ENSG00000120948 | TARDBP | Hs.300624 | 735 | PP | 992.75 | PP |
| ENSG00000028839 | TBPL1 | Hs.486507 | 170.1 | PP | 187.95 | PP |
| ENSG00000132604 | TERF2 | Hs.63335 | 241.2 | PP | 215.3 | PP |
| ENSG00000135269 | TES | Hs.533391 | 397.3 | PP | 457.65 | PP |
| ENSG00000114126 | TFDP2 | Hs.379018 | 253.2 | PP | 196.3 | PA |
| ENSG00000177426 | TGIF | Hs.373550 | 116.7 | PP | 95.35 | PM |
| ENSG00000183864 | TOB2 | Hs.474978 | 165.3 | PP | 196.4 | PP |
| ENSG00000154174 | TOMM70A | Hs.227253 | 585.1 | PP | 581.85 | PP |
| ENSG00000166340 | TPP1 | Hs.523454 | 200.3 | PP | 229.1 | PP |
| ENSG00000067167 | TRAM1 | Hs.491988 | 573.3 | PP | 894.4 | PP |
| ENSG00000140391 | TSPAN3 | Hs.5062 | 516.6 | PP | 661.45 | PP |
| ENSG00000119048 | UBE2B | Hs.385986 | 335 | PP | 417.45 | PP |
| ENSG00000131508 | UBE2D2 | Hs.108332 | 400.1 | PP | 512.05 | PP |
| ENSG00000109332 | UBE2D3 | Hs.472031 | 471.2 | PP | 635.4 | PP |
| ENSG00000132388 | UBE2G1 | Hs.462035 | 272.5 | PP | 373.45 | PP |
| ENSG00000114062 | UBE3A | Hs.22543 | 456.9 | PP | 470.75 | PP |
| ENSG00000151148 | UBE3B | Hs.374067 | 156.7 | PP | 146.6 | PP |
| ENSG00000135655 | USP15 | Hs.434951 | 197.3 | PP | 160.35 | PP |
| ENSG00000170832 | USP32 | Hs.132868 | 120.2 | PP | 118.35 | PP |
| ENSG00000077254 | USP33 | Hs.480597 | 228.7 | PP | 299.75 | PP |
| ENSG00000170242 | USP47 | Hs.501924 | 321.7 | PP | 333.3 | PP |
| ENSG00000090686 | USP48 | Hs.467524 | 366.1 | PP | 393.65 | PP |
| ENSG00000173218 | VANGL1 | Hs.515130 | 73.8 | PP | 89.5 | PA |
| ENSG00000144560 | VGLL4 | Hs.373959 | 299.7 | PP | 175.6 | PP |
| ENSG00000115561 | VPS24 | Hs.255015 | 277.6 | PP | 248.55 | PP |
| ENSG00000085433 | WDR47 | Hs.546347 | 90.4 | PP | 116.6 | PP |
| ENSG00000146457 | WTAP | Hs.446091 | 400.8 | PP | 406.6 | PP |
| ENSG00000119596 | YLPM1 | Hs.531111 | 402.5 | PP | 461.6 | PP |
| ENSG00000119801 | YPEL5 | Hs.515890 | 1377.9 | PP | 1469.25 | PP |
| ENSG00000198492 | YTHDF2 | Hs.532286 | 351.2 | PP | 449.7 | PP |
| ENSG00000166913 | YWHAB | Hs.279920 | 222.4 | PP | 288.75 | PP |
| ENSG00000107372 | ZA20D2 | Hs.406096 | 967.2 | PP | 1262.9 | PP |
| ENSG00000185650 | ZFP36L1 | Hs.85155 | 94.5 | PP | 140.7 | PP |
| ENSG00000152518 | ZFP36L2 | Hs.503093 | 4553.7 | PP | 5205.6 | PP |
| ENSG00000114514 | ZNF148 | Hs.380334 | 211.6 | PP | 226.65 | PP |
| ENSG00000179456 | ZNF238 | Hs.69997 | 235.7 | PP | 241.8 | PP |

197

| | | | | | | |
|---|---|---|---|---|---|---|
| ENSG00000168710 | AHCYL1 | Hs.485365 | 408.7 | PP | 493.4 | PP |
| ENSG00000004455 | AK2 | Hs.470907 | 303.6 | PP | 498.6 | PP |
| ENSG00000114573 | ATP6V1A | Hs.477155 | 710.1 | PP | 1105.8 | PP |
| ENSG00000115307 | AUP1 | Hs.411480 | 382.3 | PP | 325.35 | PP |
| ENSG00000179218 | CALR | Hs.515162 | 398.5 | PP | 1371.55 | PP |
| ENSG00000009307 | CSDE1 | Hs.69855 | 1494.1 | PP | 1637.9 | PP |
| ENSG00000036257 | CUL3 | Hs.372286 | 554.5 | PP | 473.25 | PP |
| ENSG00000158486 | DNAH3 | Hs.526500 | 258.2 | PP | 268.35 | PM |
| ENSG00000123908 | EIF2C2 | Hs.449415 | 150.2 | PP | 115.85 | PP |
| ENSG00000100664 | EIF5 | Hs.433702 | 96.4 | PP | 55.8 | PA |
| ENSG00000066044 | ELAVL1 | Hs.184492 | 534.6 | PP | 734.45 | PP |
| ENSG00000139146 | FAM60A | Hs.505154 | 598.1 | PP | 788 | PP |
| ENSG00000010295 | HOM-TES- | Hs.15243 | 407 | PP | 355.35 | PP |
| | 103 | | | | | |
| ENSG00000074356 | HSA277841 | Hs.120963 | 222.9 | PP | 150.05 | PP |
| ENSG00000169032 | MAP2K1 | Hs.145442 | 188.8 | PP | 162.95 | PP |
| ENSG00000121940 | MCLC | Hs.552587 | 115.5 | PP | 140.65 | PP |
| ENSG00000005810 | MYCBP2 | Hs.151411 | 187 | PP | 643.55 | PP |
| ENSG00000124789 | NUP153 | Hs.121088 | 216.4 | PP | 291.8 | PP |
| ENSG00000141447 | OSBPL1A | Hs.370725 | 204.6 | PP | 211.75 | PP |
| ENSG00000167770 | OTUB1 | Hs.473788 | 266 | PP | 155.35 | PA |
| ENSG00000112531 | QKI | Hs.510324 | 284.1 | PP | 564.45 | PP |
| ENSG00000132341 | RAN | Hs.10842 | 1434.8 | PP | 2597 | PP |
| ENSG00000120948 | TARDBP | Hs.300624 | 735 | PP | 992.75 | PP |
| ENSG00000140416 | TPM1 | Hs.133892 | 394.4 | PP | 661.4 | PP |
| ENSG00000043514 | TRIT1 | Hs.356554 | 335.4 | PP | 168.4 | PP |
| ENSG00000131508 | UBE2D2 | Hs.108332 | 400.1 | PP | 512.05 | PA |
| ENSG00000118900 | UBN1 | Hs.440219 | 138.4 | PP | 302.15 | PP |

17

| | | | | | | |
|---|---|---|---|---|---|---|
| ENSG00000165029 | ABCA1 | Hs.429294 | 175.1 | PP | 156.5 | PP |
| ENSG00000084234 | APLP2 | Hs.370247 | 1126.7 | PP | 668.55 | PP |
| ENSG00000142192 | APP | Hs.434980 | 154.6 | PP | 154.1 | PP |
| ENSG00000095139 | ARCN1 | Hs.33642 | 285 | PP | 317.05 | PP |
| ENSG00000143761 | ARF1 | Hs.286221 | 1245.2 | PP | 798.4 | PP |
| ENSG00000175220 | ARHGAP1 | Hs.138860 | 594 | PP | 502.3 | PP |
| ENSG00000111875 | ASF1A | Hs.292316 | 126.5 | PP | 114.6 | PP |
| ENSG00000154518 | ATP5G3 | Hs.429 | 1112 | PP | 1334.45 | PP |
| ENSG00000155096 | AZIN1 | Hs.459106 | 551.2 | PP | 619.2 | PP |
| ENSG00000095739 | BAMBI | Hs.533336 | 207.8 | PP | 106.85 | PP |
| ENSG00000029363 | BCLAF1 | Hs.486542 | 659.8 | PP | 1252.25 | PP |
| ENSG00000145741 | BTF3 | Hs.529798 | 6371.5 | PP | 4884.45 | PP |
| ENSG00000133639 | BTG1 | Hs.255935 | 955.9 | PP | 1057.95 | PP |
| ENSG00000119906 | C10orf6 | Hs.447458 | 154.8 | PP | 126.8 | PP |
| ENSG00000110075 | C11orf23 | Hs.503022 | 402.6 | PP | 319.5 | PA |
| ENSG00000116489 | CAPZA1 | Hs.514934 | 766.7 | PP | 1165.8 | PP |
| ENSG00000113328 | CCNG1 | Hs.79101 | 1025 | PP | 737.8 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000166226 | CCT2 | Hs.189772 | 166.1 | PP | 259.15 | PP |
| ENSG00000070831 | CDC42 | Hs.467637 | 620.1 | PP | 437.75 | PP |
| ENSG00000111642 | CHD4 | Hs.162233 | 299.5 | PP | 431.1 | PP |
| ENSG00000111596 | CNOT2 | Hs.133350 | 431.4 | PP | 399.2 | PP |
| ENSG00000166200 | COPS2 | Hs.369614 | 521.2 | PP | 700.65 | PP |
| ENSG00000167193 | CRK | Hs.461896 | 114.9 | PP | 153.9 | PP |
| ENSG00000108848 | CROP | Hs.130293 | 2166.4 | PP | 1637.55 | PA |
| ENSG00000009307 | CSDE1 | Hs.69855 | 1494.1 | PP | 1637.9 | PP |
| ENSG00000103653 | CSK | Hs.77793 | 348.1 | PP | 217.35 | PA |
| ENSG00000102974 | CTCF | Hs.368367 | 506.1 | PP | 535.55 | PP |
| ENSG00000036257 | CUL3 | Hs.372286 | 554.5 | PP | 473.25 | PP |
| ENSG00000160967 | CUTL1 | Hs.438974 | 333.3 | PP | 412.55 | PP |
| ENSG00000071626 | DAZAP1 | Hs.222510 | 49.7 | PP | 130.7 | PA |
| ENSG00000183283 | DAZAP2 | Hs.369761 | 1267.2 | PP | 976.7 | PP |
| ENSG00000124487 | DDX3X | Hs.380774 | 769.8 | PP | 1229 | PP |
| ENSG00000108654 | DDX5 | Hs.279806 | 3052 | PP | 3508.95 | PP |
| ENSG00000109606 | DHX15 | Hs.5683 | 1219.8 | PP | 1620.75 | PP |
| ENSG00000112242 | E2F3 | Hs.269408 | 295.6 | PP | 585.95 | PP |
| ENSG00000092847 | EIF2C1 | Hs.22867 | 292.5 | PP | 296.95 | PP |
| ENSG00000104131 | EIF3S1 | Hs.404056 | 340.4 | PP | 365.05 | PP |
| ENSG00000107581 | EIF3S10 | Hs.523299 | 522.9 | PP | 586.55 | PP |
| ENSG00000151247 | EIF4E | Hs.249718 | 156.7 | PP | 224 | PP |
| ENSG00000110321 | EIF4G2 | Hs.183684 | 2137 | PP | 2701.9 | PP |
| ENSG00000106462 | EZH2 | Hs.444082 | 162.9 | PP | 216.25 | PP |
| ENSG00000139146 | FAM60A | Hs.505154 | 598.1 | PP | 788 | PP |
| ENSG00000173120 | FBXL11 | Hs.124147 | 237 | PP | 192.5 | PP |
| ENSG00000118564 | FBXL5 | Hs.479208 | 410.6 | PP | 319.15 | PP |
| ENSG00000138081 | FBXO11 | Hs.553547 | 155.7 | PP | 259.35 | PP |
| ENSG00000022267 | FHL1 | Hs.435369 | 5240 | PP | 3151.75 | PP |
| ENSG00000170345 | FOS | Hs.25647 | 1254.3 | PP | 1832.65 | PP |
| ENSG00000150907 | FOXO1A | Hs.370666 | 253.6 | PP | 140.95 | PP |
| ENSG00000114861 | FOXP1 | Hs.431498 | 322.7 | PP | 410.3 | PP |
| ENSG00000163430 | FSTL1 | Hs.269512 | 408.6 | PP | 227.95 | PP |
| ENSG00000143641 | GALNT2 | Hs.300834 | 321.4 | PP | 333.55 | PP |
| ENSG00000057608 | GDI2 | Hs.299055 | 198.2 | PP | 260.65 | PP |
| ENSG00000115806 | GORASP2 | Hs.431317 | 359.7 | PP | 304.85 | PP |
| ENSG00000110768 | GTF2H1 | Hs.523510 | 167.7 | PP | 191.7 | PP |
| ENSG00000143321 | HDGF | Hs.506748 | 243.6 | PP | 858.8 | PP |
| ENSG00000051108 | HERPUD1 | Hs.146393 | 253 | PP | 588.95 | PP |
| ENSG00000010818 | HIVEP2 | Hs.510172 | 149.6 | PP | 157.9 | PP |
| ENSG00000029993 | HMGB3 | Hs.19114 | 309 | PP | 460.6 | PP |
| ENSG00000122566 | HNRPA2B1 | Hs.487774 | 1802.2 | PP | 2268.3 | PP |
| ENSG00000170144 | HNRPA3 | Hs.516539 | 375.5 | PP | 558.6 | PP |
| ENSG00000096746 | HNRPH3 | Hs.499891 | 408.7 | PP | 635.5 | PP |
| ENSG00000099783 | HNRPM | Hs.465808 | 968.5 | PP | 880.2 | PP |
| ENSG00000125944 | HNRPR | Hs.373763 | 839.4 | PP | 980.1 | PP |
| ENSG00000153187 | HNRPU | Hs.166463 | 470.3 | PP | 887.6 | PP |
| ENSG00000105323 | HNRPUL1 | Hs.155218 | 406.9 | PP | 401.15 | PP |
| ENSG00000173744 | HRB | Hs.443507 | 202.6 | PP | 405.7 | PP |
| ENSG00000067829 | IDH3G | Hs.410197 | 249.2 | PP | 156.95 | PA |
| ENSG00000070061 | IKBKAP | Hs.494738 | 157.6 | PP | 208.95 | PP |
| ENSG00000115548 | JMJD1A | Hs.531819 | 264 | PP | 207.05 | PP |
| ENSG00000177606 | JUN | Hs.525704 | 1300.5 | PP | 2286.95 | PP |
| ENSG00000170852 | KBTBD2 | Hs.372541 | 412.2 | PP | 381.65 | PP |
| ENSG00000121774 | KHDRBS1 | Hs.445893 | 1363 | PP | 1395.2 | PP |
| ENSG00000102753 | KPNA3 | Hs.527919 | 250.8 | PP | 391.35 | PP |
| ENSG00000108424 | KPNB1 | Hs.532793 | 415.1 | PP | 709.85 | PP |
| ENSG00000160789 | LMNA | Hs.491359 | 309.3 | PP | 352.9 | PP |
| ENSG00000121104 | LOC81558 | Hs.514308 | 3067.5 | PP | 1370.7 | PP |
| ENSG00000163818 | LZTFL1 | Hs.30824 | 199 | PP | 253.95 | PP |
| ENSG00000173327 | MAP3K11 | Hs.502872 | 355.1 | PP | 326.8 | PP |
| ENSG00000104814 | MAP4K1 | Hs.95424 | 586.5 | PP | 231.45 | PP |
| ENSG00000101367 | MAPRE1 | Hs.472437 | 196.1 | PP | 206.85 | PM |
| ENSG00000136536 | 7-Mar | Hs.529272 | 175.3 | PP | 173.1 | PP |
| ENSG00000165406 | 8-Mar | Hs.499489 | 295.9 | PP | 295.85 | PP |
| ENSG00000166986 | MARS | Hs.355867 | 460.7 | PP | 614.95 | PP |
| ENSG00000168906 | MAT2A | Hs.516157 | 1011.4 | PP | 738.6 | PP |
| ENSG00000038274 | MAT2B | Hs.54642 | 609.4 | PP | 461.9 | PP |
| ENSG00000081189 | MEF2C | Hs.444409 | 426 | PP | 437.85 | PP |
| ENSG00000143995 | MEIS1 | Hs.526754 | 452.1 | PP | 262.65 | PP |
| ENSG00000133606 | MKRN1 | Hs.490347 | 392.7 | PP | 350.15 | PP |
| ENSG00000100330 | MTMR3 | Hs.474536 | 204.7 | PP | 287 | PP |
| ENSG00000108389 | MTMR4 | Hs.514373 | 330.7 | PP | 407.2 | PP |
| ENSG00000133026 | MYH10 | Hs.16355 | 375.1 | PP | 362.5 | PP |
| ENSG00000136504 | MYST2 | Hs.21907 | 331.7 | PP | 192.25 | PP |
| ENSG00000166579 | NDEL1 | Hs.372123 | 218.3 | PP | 187.2 | PP |
| ENSG00000125356 | NDUFA1 | Hs.534168 | 2121.1 | PP | 1598.7 | PP |
| ENSG00000069869 | NEDD4 | Hs.1565 | 186.1 | PP | 193.9 | PP |
| ENSG00000082641 | NFE2L1 | Hs.514284 | 735.5 | PP | 726.35 | PP |
| ENSG00000120837 | NFYB | Hs.84928 | 204.1 | PP | 272.85 | PP |
| ENSG00000164190 | NIPBL | Hs.481927 | 165.1 | PP | 145.15 | PP |
| ENSG00000137420 | NOL7 | Hs.549161 | 1095.5 | PP | 1222.05 | PP |
| ENSG00000181163 | NPM1 | Hs.519452 | 8905.6 | PP | 11897.7 | PP |
| ENSG00000181163 | NPM1 | Hs.519452 | 2088.9 | PP | 2381.65 | PP |
| ENSG00000153234 | NR4A2 | Hs.165258 | 124.6 | PP | 242.85 | PP |
| ENSG00000046651 | OFD1 | Hs.6483 | 1022.7 | PP | 692.15 | PP |
| ENSG00000141447 | OSBPL1A | Hs.370725 | 204.6 | PP | 211.75 | PP |
| ENSG00000090060 | PAPOLA | Hs.253726 | 307.4 | PP | 286 | PP |
| ENSG00000115421 | PAPOLG | Hs.387471 | 153.2 | PP | 91.25 | PP |
| ENSG00000157212 | PAXIP1 | Hs.443881 | 139.4 | PP | 130.7 | PP |
| ENSG00000056661 | PCGF2 | Hs.371617 | 231.6 | PP | 160.8 | PP |
| ENSG00000185619 | PCGF3 | Hs.144309 | 192.1 | PP | 237.55 | PP |
| ENSG00000059758 | PCTK2 | Hs.506415 | 118.3 | PP | 220.9 | PP |
| ENSG00000164040 | PGRMC2 | Hs.507910 | 89.1 | PP | 87.85 | PM |
| ENSG00000025293 | PHF20 | Hs.517044 | 249.8 | PP | 173.65 | PP |
| ENSG00000181191 | PJA1 | Hs.522679 | 316.3 | PP | 332.1 | PP |
| ENSG00000136040 | PLXNC1 | Hs.551522 | 354.6 | PP | 387.1 | PP |
| ENSG00000100941 | PNN | Hs.409965 | 839.5 | PP | 617.3 | PP |
| ENSG00000163806 | PPP1CB | Hs.468018 | 65.7 | PP | 135.05 | PP |
| ENSG00000113575 | PPP2CA | Hs.483408 | 360 | PP | 370.4 | PP |
| ENSG00000107758 | PPP3CB | Hs.500067 | 318.9 | PP | 241.25 | PP |
| ENSG00000119414 | PPP6C | Hs.495128 | 297.5 | PP | 557.55 | PP |
| ENSG00000116731 | PRDM2 | Hs.371823 | 329.6 | PP | 175.6 | PP |
| ENSG00000100519 | PSMC6 | Hs.156171 | 143.7 | PP | 216.4 | PP |
| ENSG00000125818 | PSMF1 | Hs.471917 | 254.5 | PP | 259.45 | PP |
| ENSG00000175354 | PTPN2 | Hs.123352 | 759.9 | PP | 273 | PP |
| ENSG00000134644 | PUM1 | Hs.281707 | 378.4 | PP | 422.3 | PP |
| ENSG00000130508 | PXDN | Hs.332197 | 280.3 | PP | 114.7 | PA |
| ENSG00000112531 | QKI | Hs.510324 | 284.1 | PP | 564.45 | PP |
| ENSG00000103769 | RAB11A | Hs.321541 | 1087.7 | PP | 1020.15 | PP |
| ENSG00000119396 | RAB14 | Hs.371563 | 140.5 | PP | 198.15 | PP |
| ENSG00000111540 | RAB5B | Hs.77690 | 234.1 | PP | 216.45 | PP |
| ENSG00000175582 | RAB6A | Hs.503222 | 371.4 | PP | 365.75 | PP |
| ENSG00000119318 | RAD23B | Hs.521640 | 156.2 | PP | 211.05 | PP |
| ENSG00000132341 | RAN | Hs.10842 | 1434.8 | PP | 2597 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000123091 | RNF11 | Hs.309641 | 125.3 | PP | 161.5 | PP |
| ENSG00000127870 | RNF6 | Hs.136885 | 74.1 | PP | 122.9 | PP |
| ENSG00000043093 | RP42 | Hs.104613 | 236.5 | PP | 204.45 | PP |
| ENSG00000100784 | RPS6KA5 | Hs.510225 | 152.1 | PP | 132.75 | PP |
| ENSG00000148484 | RSU1 | Hs.524161 | 298.8 | PP | 294.55 | PP |
| ENSG00000159216 | RUNX1 | Hs.149261 | 108.8 | PP | 211.2 | PP |
| ENSG00000163602 | RYBP | Hs.7910 | 398 | PP | 462.8 | PP |
| ENSG00000079332 | SARA1 | Hs.499960 | 233.7 | PP | 442.85 | PP |
| ENSG00000151967 | SCHIP1 | Hs.134665 | 497 | PP | 417.95 | PP |
| ENSG00000179833 | SERTAD2 | Hs.77293 | 498.4 | PP | 556.2 | PP |
| ENSG00000119335 | SET | Hs.436687 | 2967 | PP | 4307.1 | PP |
| ENSG00000162105 | SHANK2 | Hs.268726 | 137.1 | PP | 128.55 | PP |
| ENSG00000096717 | SIRT1 | Hs.369779 | 133.8 | PP | 124.9 | PP |
| ENSG00000113558 | SKP1A | Hs.171626 | 657.2 | PP | 969.15 | PP |
| ENSG00000075415 | SLC25A3 | Hs.290404 | 2025.9 | PP | 2749.5 | PP |
| ENSG00000059804 | SLC2A3 | Hs.419240 | 275.9 | PP | 351.5 | PP |
| ENSG00000080503 | SMARCA2 | Hs.298990 | 264.4 | PP | 201.3 | PP |
| ENSG00000173473 | SMARCC1 | Hs.476179 | 487 | PP | 483.35 | PP |
| ENSG00000120833 | SOCS2 | Hs.485572 | 715.8 | PP | 806.65 | PP |
| ENSG00000159140 | SON | Hs.517262 | 408.8 | PP | 467.15 | PP |
| ENSG00000137642 | SORL1 | Hs.368592 | 2264.1 | PP | 1084.65 | PP |
| ENSG00000124766 | SOX4 | Hs.357901 | 2351.7 | PP | 2500.65 | PP |
| ENSG00000008294 | SPAG9 | Hs.463439 | 180.3 | PP | 209.9 | PP |
| ENSG00000168610 | STAT3 | Hs.463059 | 401.7 | PP | 390.05 | PP |
| ENSG00000132718 | SYT11 | Hs.32984 | 106.3 | PP | 102.95 | PP |
| ENSG00000108264 | TADA2L | Hs.500066 | 108 | PP | 97.9 | PA |
| ENSG00000120948 | TARDBP | Hs.300624 | 735 | PP | 992.75 | PP |
| ENSG00000106052 | TAX1BP1 | Hs.34576 | 550 | PP | 486.2 | PP |
| ENSG00000113649 | TCERG1 | Hs.443465 | 196.5 | PP | 257.25 | PP |
| ENSG00000101337 | TM9SF4 | Hs.529360 | 129.2 | PP | 121.3 | PP |
| ENSG00000170348 | TMP21 | Hs.74137 | 293.5 | PP | 280.3 | PA |
| ENSG00000083312 | TNPO1 | Hs.482497 | 483 | PP | 750.85 | PP |
| ENSG00000102804 | TSC22D1 | Hs.450972 | 1492.8 | PP | 2518.9 | PP |
| ENSG00000117289 | TXNIP | Hs.533977 | 698.8 | PP | 1099.3 | PP |
| ENSG00000150991 | UBC | Hs.520348 | 2138.1 | PP | 2607.15 | PP |
| ENSG00000119048 | UBE2B | Hs.385986 | 335 | PP | 417.45 | PP |
| ENSG00000170035 | UBE2E3 | Hs.470804 | 311.5 | PP | 258.25 | PP |
| ENSG00000009335 | UBE3C | Hs.118351 | 67.5 | PP | 175.9 | PM |
| ENSG00000077254 | USP33 | Hs.480597 | 228.7 | PP | 299.75 | PP |
| ENSG00000134215 | VAV3 | Hs.267659 | 615.4 | PP | 341.3 | PP |
| ENSG00000026025 | VIM | Hs.533317 | 4064.1 | PP | 2437.95 | PP |
| ENSG00000132716 | WDR42A | Hs.492236 | 195.9 | PP | 233.1 | PP |
| ENSG00000166913 | YWHAB | Hs.279920 | 222.4 | PP | 288.75 | PP |
| ENSG00000107372 | ZA20D2 | Hs.406096 | 967.2 | PP | 1262.9 | PP |
| ENSG00000185650 | ZFP36L1 | Hs.85155 | 94.5 | PP | 140.7 | PP |
| ENSG00000015171 | ZMYND11 | Hs.292265 | 403.3 | PP | 784.1 | PP |
| ENSG00000114514 | ZNF148 | Hs.380334 | 211.6 | PP | 226.65 | PP |
| ENSG00000169714 | ZNF9 | Hs.518249 | 1408.6 | PP | 1399.55 | PP |

20

| | | | | | | |
|---|---|---|---|---|---|---|
| ENSG00000165029 | ABCA1 | Hs.429294 | 175.1 | PP | 156.5 | PP |
| ENSG00000170776 | AKAP13 | Hs.459211 | 192.3 | PP | 198.75 | PA |
| ENSG00000117020 | AKT3 | Hs.498292 | 246.4 | PP | 259.35 | PP |
| ENSG00000133805 | AMPD3 | Hs.501890 | 253.6 | PP | 285.4 | PP |
| ENSG00000107104 | ANKRD15 | Hs.493272 | 235.2 | PP | 131.45 | PP |
| ENSG00000182287 | AP1S2 | Hs.121592 | 292 | PP | 269.6 | PP |
| ENSG00000142192 | APP | Hs.434980 | 154.6 | PP | 154.1 | PP |
| ENSG00000143761 | ARF1 | Hs.286221 | 1245.2 | PP | 798.4 | PP |
| ENSG00000175220 | ARHGAP1 | Hs.138860 | 594 | PP | 502.3 | PP |
| ENSG00000104880 | ARHGEF18 | Hs.465763 | 366 | PP | 254.9 | PA |
| ENSG00000054267 | ARID4B | Hs.533633 | 227 | PP | 252.45 | PP |
| ENSG00000188042 | ARL7 | Hs.111554 | 240.7 | PP | 203.4 | PP |
| ENSG00000111875 | ASF1A | Hs.292316 | 126.5 | PP | 114.6 | PP |
| ENSG00000095739 | BAMBI | Hs.533336 | 207.8 | PP | 106.85 | PP |
| ENSG00000029363 | BCLAF1 | Hs.486542 | 659.8 | PP | 1252.25 | PP |
| ENSG00000133639 | BTG1 | Hs.255935 | 955.9 | PP | 1057.95 | PP |
| ENSG00000159388 | BTG2 | Hs.519162 | 588.2 | PP | 538.3 | PP |
| ENSG00000154640 | BTG3 | Hs.473420 | 315.6 | PP | 378.5 | PP |
| ENSG00000110075 | C11orf23 | Hs.503022 | 402.6 | PP | 319.5 | PA |
| ENSG00000141452 | C18orf8 | Hs.529006 | 232.8 | PP | 242.05 | PP |
| ENSG00000105176 | C19orf2 | Hs.466391 | 501.6 | PP | 404.65 | PP |
| ENSG00000117616 | C1orf63 | Hs.259412 | 1202.1 | PP | 1000.85 | PP |
| ENSG00000110888 | C1QDC1 | Hs.234355 | 291.3 | PP | 261.05 | PP |
| ENSG00000127022 | CANX | Hs.529890 | 1003.1 | PP | 2097.75 | PP |
| ENSG00000127022 | CANX | Hs.529890 | 253.4 | PP | 361.2 | PP |
| ENSG00000118971 | CCND2 | Hs.376071 | 1114.1 | PP | 1244.6 | PA |
| ENSG00000113328 | CCNG1 | Hs.79101 | 1025 | PP | 737.8 | PP |
| ENSG00000138764 | CCNG2 | Hs.13291 | 120.9 | PP | 268.8 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000110848 | CD69 | Hs.208854 | 1631.5 | PP | 1082.1 | PP |
| ENSG00000143126 | CELSR2 | Hs.57652 | 215.4 | PP | 148.65 | PP |
| ENSG00000177200 | CHD9 | Hs.530936 | 222.6 | PP | 263 | PP |
| ENSG00000131873 | CHSY1 | Hs.110488 | 404.5 | PP | 490.95 | PP |
| ENSG00000162368 | CMPK | Hs.11463 | 333.8 | PP | 336.55 | PP |
| ENSG00000198791 | CNOT7 | Hs.170553 | 250.3 | PP | 508.5 | PP |
| ENSG00000121058 | COIL | Hs.532795 | 134.2 | PP | 210.15 | PP |
| ENSG00000167193 | CRK | Hs.461896 | 114.9 | PP | 153.9 | PP |
| ENSG00000108848 | CROP | Hs.130293 | 2166.4 | PP | 1637.55 | PP |
| ENSG00000103653 | CSK | Hs.77793 | 348.1 | PP | 217.35 | PA |
| ENSG00000102974 | CTCF | Hs.368367 | 506.1 | PP | 535.55 | PP |
| ENSG00000048740 | CUGBP2 | Hs.309288 | 159.6 | PP | 334.5 | PP |
| ENSG00000183283 | DAZAP2 | Hs.369761 | 1267.2 | PP | 976.7 | PP |
| ENSG00000124487 | DDX3X | Hs.380774 | 769.8 | PP | 1229 | PP |
| ENSG00000108654 | DDX5 | Hs.279806 | 3052 | PP | 3508.95 | PP |
| ENSG00000105993 | DNAJB6 | Hs.490745 | 189.7 | PP | 348.5 | PP |
| ENSG00000128590 | DNAJB9 | Hs.6790 | 211.5 | PP | 433.55 | PP |
| ENSG00000092964 | DPYSL2 | Hs.173381 | 505.1 | PP | 495.1 | PP |
| ENSG00000139318 | DUSP6 | Hs.298654 | 166.6 | PP | 316.85 | PP |
| ENSG00000157540 | DYRK1A | Hs.368240 | 367.8 | PP | 751.2 | PP |
| ENSG00000112242 | E2F3 | Hs.269408 | 295.6 | PP | 585.95 | PP |
| ENSG00000104517 | EDD1 | Hs.492445 | 254.6 | PP | 227.15 | PP |
| ENSG00000120738 | EGR1 | Hs.326035 | 128.6 | PP | 290.15 | PP |
| ENSG00000092847 | EIF2C1 | Hs.22867 | 292.5 | PP | 296.95 | PP |
| ENSG00000104131 | EIF3S1 | Hs.404056 | 340.4 | PP | 365.05 | PP |
| ENSG00000107581 | EIF3S10 | Hs.523299 | 522.9 | PP | 586.55 | PP |
| ENSG00000151247 | EIF4E | Hs.249718 | 156.7 | PP | 224 | PP |
| ENSG00000110321 | EIF4G2 | Hs.183684 | 2137 | PP | 2701.9 | PP |
| ENSG00000109381 | ELF2 | Hs.260903 | 150.8 | PP | 152.4 | PP |
| ENSG00000111145 | ELK3 | Hs.46523 | 372.1 | PP | 280.75 | PP |
| ENSG00000050405 | EPLIN | Hs.525419 | 265.5 | PP | 309.7 | PP |
| ENSG00000124882 | EREG | Hs.115263 | 351.8 | PP | 535.15 | PP |
| ENSG00000120705 | ETF1 | Hs.483494 | 253.8 | PP | 461.55 | PP |
| ENSG00000108799 | EZH1 | Hs.194669 | 211.4 | PP | 194.45 | PP |
| ENSG00000106462 | EZH2 | Hs.444082 | 162.9 | PP | 216.25 | PP |
| ENSG00000119979 | FAM45B /// | Hs.434241 | 554.8 | PP | 690.2 | PP |
| | FAM45A | | | | | |
| ENSG00000139146 | FAM60A | Hs.505154 | 598.1 | PP | 788 | PP |
| ENSG00000173120 | FBXL11 | Hs.124147 | 237 | PP | 192.5 | PP |
| ENSG00000118564 | FBXL5 | Hs.479208 | 410.6 | PP | 319.15 | PP |
| ENSG00000138081 | FBXO11 | Hs.553547 | 155.7 | PP | 259.35 | PP |
| ENSG00000143756 | FBXO28 | Hs.64691 | 106.1 | PP | 70.5 | PA |
| ENSG00000022267 | FHL1 | Hs.435369 | 5240 | PP | 3151.75 | PP |
| ENSG00000111011 | FLJ11021 | Hs.432996 | 202 | PP | 392.6 | PP |
| ENSG00000109920 | FNBP4 | Hs.6834 | 501.2 | PP | 497.2 | PP |
| ENSG00000198815 | FOXJ3 | Hs.26023 | 458.7 | PP | 380.45 | PP |
| ENSG00000114416 | FXR1 | Hs.478407 | 143.9 | PP | 155.75 | PP |
| ENSG00000145907 | G3BP | Hs.3353 | 167.7 | PP | 319.25 | PP |
| ENSG00000104064 | GABPB2 | Hs.546852 | 129.3 | PP | 183.75 | PM |
| ENSG00000143641 | GALNT2 | Hs.300834 | 321.4 | PP | 333.55 | PP |
| ENSG00000196700 | GM632 | Hs.551552 | 103.5 | PP | 145.05 | PP |
| ENSG00000113384 | GOLPH3 | Hs.408909 | 585.8 | PP | 782.9 | PP |
| ENSG00000112406 | HECA | Hs.197644 | 352.2 | PP | 377.45 | PP |
| ENSG00000100644 | HIF1A | Hs.509554 | 866.5 | PP | 1037.1 | PP |
| ENSG00000010818 | HIVEP2 | Hs.510172 | 149.6 | PP | 157.9 | PP |
| ENSG00000029993 | HMGB3 | Hs.19114 | 309 | PP | 460.6 | PP |
| ENSG00000122566 | HNRPA2B1 | Hs.487774 | 1802.2 | PP | 2268.3 | PP |
| ENSG00000096746 | HNRPH3 | Hs.499891 | 408.7 | PP | 635.5 | PP |
| ENSG00000153187 | HNRPU | Hs.166463 | 470.3 | PP | 887.6 | PP |
| ENSG00000164164 | HSHIN1 | Hs.270851 | 303.1 | PP | 312.65 | PP |
| ENSG00000067829 | IDH3G | Hs.410197 | 249.2 | PP | 156.95 | PA |
| ENSG00000168556 | ING2 | Hs.107153 | 177 | PP | 151.85 | PP |
| ENSG00000125347 | IRF1 | Hs.436061 | 467.7 | PP | 299.6 | PP |
| ENSG00000116679 | IVNS1ABP | Hs.497183 | 251.6 | PP | 288.25 | PP |
| ENSG00000117139 | JARID1B | Hs.443650 | 203.6 | PP | 353.6 | PP |
| ENSG00000115548 | JMJD1A | Hs.531819 | 264 | PP | 207.05 | PP |
| ENSG00000177606 | JUN | Hs.525704 | 1300.5 | PP | 2286.95 | PP |
| ENSG00000137944 | KAT3 | Hs.481898 | 518.7 | PP | 319.8 | PP |
| ENSG00000121774 | KHDRBS1 | Hs.445893 | 1363 | PP | 1395.2 | PP |
| ENSG00000143702 | KIAA0470 | Hs.533635 | 146.2 | PP | 325 | PP |
| ENSG00000102753 | KPNA3 | Hs.527919 | 250.8 | PP | 391.35 | PP |
| ENSG00000068697 | LAPTM4A | Hs.467807 | 1059.2 | PP | 1145.5 | PP |
| ENSG00000172292 | LASS6 | Hs.506829 | 536.2 | PP | 463.8 | PP |
| ENSG00000121104 | LOC81558 | Hs.514308 | 3067.5 | PP | 1370.7 | PP |
| ENSG00000123684 | LPGAT1 | Hs.497674 | 106 | PP | 114.3 | PP |
| ENSG00000171492 | LRRC8D | Hs.482087 | 543.3 | PP | 374.45 | PP |
| ENSG00000163818 | LZTFL1 | Hs.30824 | 199 | PP | 253.95 | PP |
| ENSG00000003056 | M6PR | Hs.134084 | 566.2 | PP | 575.55 | PP |
| ENSG00000187391 | MAGI2 | Hs.488945 | 93 | PP | 59.4 | PA |
| ENSG00000173327 | MAP3K11 | Hs.502872 | 355.1 | PP | 326.8 | PP |
| ENSG00000197442 | MAP3K5 | Hs.186486 | 227.6 | PP | 272.25 | PP |
| ENSG00000104814 | MAP4K1 | Hs.95424 | 586.5 | PP | 231.45 | PP |
| ENSG00000135525 | MAP7 | Hs.486548 | 325.7 | PP | 390.1 | PP |
| ENSG00000101367 | MAPRE1 | Hs.472437 | 196.1 | PP | 206.85 | PM |
| ENSG00000166974 | MAPRE2 | Hs.532824 | 857.4 | PP | 715.5 | PP |
| ENSG00000145495 | 6-Mar | Hs.432862 | 597.3 | PP | 536.05 | PP |
| ENSG00000165406 | 8-Mar | Hs.499489 | 295.9 | PP | 295.85 | PP |
| ENSG00000038274 | MAT2B | Hs.54642 | 609.4 | PP | 461.9 | PP |
| ENSG00000015479 | MATR3 | Hs.268939 | 252.4 | PP | 382.35 | PP |
| ENSG00000124733 | MEA1 | Hs.278362 | 452.7 | PP | 460.2 | PP |
| ENSG00000081189 | MEF2C | Hs.444409 | 426 | PP | 437.85 | PP |
| ENSG00000138092 | MGC11266 | Hs.546428 | 152.2 | PP | 90.95 | PA |
| ENSG00000198408 | MGEA5 | Hs.500842 | 363.7 | PP | 363.05 | PP |
| ENSG00000133606 | MKRN1 | Hs.490347 | 392.7 | PP | 350.15 | PP |
| ENSG00000185787 | MORF4L1 | Hs.374503 | 3350.8 | PP | 4457.05 | PP |
| ENSG00000100330 | MTMR3 | Hs.474536 | 204.7 | PP | 287 | PP |
| ENSG00000108389 | MTMR4 | Hs.514373 | 330.7 | PP | 407.2 | PP |
| ENSG00000005810 | MYCBP2 | Hs.151411 | 187 | PP | 643.55 | PP |
| ENSG00000132780 | NASP | Hs.319334 | 273.5 | PP | 312.4 | PP |
| ENSG00000158747 | NBL1 | Hs.439671 | 370.3 | PP | 360.6 | PA |
| ENSG00000166579 | NDEL1 | Hs.372123 | 218.3 | PP | 187.2 | PP |
| ENSG00000125356 | NDUFA1 | Hs.534168 | 2121.1 | PP | 1598.7 | PP |
| ENSG00000069869 | NEDD4 | Hs.1565 | 186.1 | PP | 193.9 | PP |
| ENSG00000120837 | NFYB | Hs.84928 | 204.1 | PP | 272.85 | PP |
| ENSG00000137420 | NOL7 | Hs.549161 | 1095.5 | PP | 1222.05 | PP |
| ENSG00000149308 | NPAT | Hs.171061 | 171.1 | PP | 140.65 | PP |
| ENSG00000153234 | NR4A2 | Hs.165258 | 124.6 | PP | 242.85 | PP |
| ENSG00000141447 | OSBPL1A | Hs.370725 | 204.6 | PP | 211.75 | PP |
| ENSG00000090060 | PAPOLA | Hs.253726 | 307.4 | PP | 286 | PP |
| ENSG00000157212 | PAXIP1 | Hs.443881 | 139.4 | PP | 130.7 | PP |
| ENSG00000197111 | PCBP2 | Hs.546271 | 2032.3 | PP | 1761.15 | PP |
| ENSG00000185619 | PCGF3 | Hs.144309 | 192.1 | PP | 237.55 | PP |
| ENSG00000059758 | PCTK2 | Hs.506415 | 118.3 | PP | 220.9 | PP |
| ENSG00000114209 | PDCD10 | Hs.478150 | 463.6 | PP | 560.4 | PP |
| ENSG00000112511 | PHF1 | Hs.166204 | 903.7 | PP | 604.1 | PP |
| ENSG00000166037 | PIG8 | Hs.101014 | 356.2 | PP | 374.1 | PP |
| ENSG00000145675 | PIK3R1 | Hs.132225 | 249.1 | PP | 181 | PP |
| ENSG00000171033 | PKIA | Hs.433700 | 88.6 | PP | 131.05 | PP |
| ENSG00000136040 | PLXNC1 | Hs.551522 | 354.6 | PP | 387.1 | PP |
| ENSG00000100941 | PNN | Hs.409965 | 839.5 | PP | 617.3 | PP |
| ENSG00000113575 | PPP2CA | Hs.483408 | 360 | PP | 370.4 | PP |
| ENSG00000078304 | PPP2R5C | Hs.368264 | 174.9 | PP | 165.25 | PP |
| ENSG00000138814 | PPP3CA | Hs.435512 | 122.5 | PP | 205 | PP |
| ENSG00000107758 | PPP3CB | Hs.500067 | 318.9 | PP | 241.25 | PP |
| ENSG00000119414 | PPP6C | Hs.495128 | 297.5 | PP | 557.55 | PP |
| ENSG00000116731 | PRDM2 | Hs.371823 | 329.6 | PP | 175.6 | PP |
| ENSG00000005249 | PRKAR2B | Hs.433068 | 200.7 | PP | 237.75 | PP |
| ENSG00000171862 | PTEN | Hs.500466 | 353.7 | PP | 334.4 | PP |
| ENSG00000112245 | PTP4A1 | Hs.227777 | 439.5 | PP | 431.2 | PP |
| ENSG00000175354 | PTPN2 | Hs.123352 | 759.9 | PP | 273 | PP |
| ENSG00000134644 | PUM1 | Hs.281707 | 378.4 | PP | 422.3 | PP |
| ENSG00000185129 | PURA | Hs.443121 | 149.5 | PP | 224.95 | PP |
| ENSG00000130508 | PXDN | Hs.332197 | 280.3 | PP | 114.7 | PA |
| ENSG00000112531 | QKI | Hs.510324 | 284.1 | PP | 564.45 | PP |
| ENSG00000103769 | RAB11A | Hs.321541 | 1087.7 | PP | 1020.15 | PP |
| ENSG00000119396 | RAB14 | Hs.371563 | 140.5 | PP | 198.15 | PP |
| ENSG00000111737 | RAB35 | Hs.524788 | 394.6 | PP | 431.2 | PP |
| ENSG00000111540 | RAB5B | Hs.77690 | 234.1 | PP | 216.45 | PP |
| ENSG00000175582 | RAB6A | Hs.503222 | 371.4 | PP | 365.75 | PP |
| ENSG00000119318 | RAD23B | Hs.521640 | 156.2 | PP | 211.05 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000101265 | RASSF2 | Hs.379970 | 129.4 | PP | 203.9 | PP |
| ENSG00000139687 | RB1 | Hs.408528 | 152.7 | PP | 153.35 | PP |
| ENSG00000102054 | RBBP7 | Hs.495755 | 942.6 | PP | 1356.35 | PP |
| ENSG00000103479 | RBL2 | Hs.513609 | 203.5 | PP | 188.25 | PP |
| ENSG00000089902 | RCOR1 | Hs.510521 | 118.3 | PP | 144.05 | PP |
| ENSG00000082996 | RNF13 | Hs.12333 | 230.9 | PP | 344.6 | PP |
| ENSG00000063978 | RNF4 | Hs.66394 | 115.8 | PP | 325.65 | PP |
| ENSG00000127870 | RNF6 | Hs.136885 | 74.1 | PP | 122.9 | PP |
| ENSG00000043093 | RP42 | Hs.104613 | 236.5 | PP | 204.45 | PP |
| ENSG00000117748 | RPA2 | Hs.79411 | 289.9 | PP | 378.45 | PP |
| ENSG00000117676 | RPS6KA1 | Hs.149957 | 288.1 | PP | 176.7 | PA |
| ENSG00000100784 | RPS6KA5 | Hs.510225 | 152.1 | PP | 132.75 | PP |
| ENSG00000159216 | RUNX1 | Hs.149261 | 108.8 | PP | 211.2 | PP |
| ENSG00000163602 | RYBP | Hs.7910 | 398 | PP | 462.8 | PP |
| ENSG00000079332 | SARA1 | Hs.499960 | 233.7 | PP | 442.85 | PP |
| ENSG00000140497 | SCAMP2 | Hs.458917 | 411.8 | PP | 495 | PP |
| ENSG00000137575 | SDCBP | Hs.200804 | 934 | PP | 955.25 | PP |
| ENSG00000120742 | SERP1 | Hs.518326 | 285.7 | PP | 343.4 | PP |
| ENSG00000179833 | SERTAD2 | Hs.77293 | 498.4 | PP | 556.2 | PP |
| ENSG00000119335 | SET | Hs.436687 | 2967 | PP | 4307.1 | PP |
| ENSG00000112081 | SFRS3 | Hs.405144 | 884.9 | PP | 923.3 | PP |
| ENSG00000096717 | SIRT1 | Hs.369779 | 133.8 | PP | 124.9 | PP |
| ENSG00000134294 | SLC38A2 | Hs.221847 | 634.3 | PP | 1026.55 | PP |
| ENSG00000080493 | SLC4A4 | Hs.5462 | 124.5 | PP | 268.4 | PP |
| ENSG00000080503 | SMARCA2 | Hs.298990 | 264.4 | PP | 201.3 | PP |
| ENSG00000159140 | SON | Hs.517262 | 408.8 | PP | 467.15 | PP |
| ENSG00000137642 | SORL1 | Hs.368592 | 2264.1 | PP | 1084.65 | PP |
| ENSG00000115904 | SOS1 | Hs.278733 | 144 | PP | 169.4 | PP |
| ENSG00000196700 | SOX18 | Hs.8619 | 1264.6 | PP | 2674 | PP |
| ENSG00000096063 | SRPK1 | Hs.443861 | 231.6 | PP | 200.5 | PP |
| ENSG00000168610 | STAT3 | Hs.463059 | 401.7 | PP | 390.05 | PP |
| ENSG00000112079 | STK38 | Hs.409578 | 365.8 | PP | 304.4 | PP |
| ENSG00000132718 | SYT11 | Hs.32984 | 106.3 | PP | 102.95 | PP |
| ENSG00000106052 | TAX1BP1 | Hs.34576 | 550 | PP | 486.2 | PP |
| ENSG00000100207 | TCF20 | Hs.475018 | 251.3 | PP | 281.15 | PP |
| ENSG00000152291 | TGOLN2 | Hs.14894 | 430.2 | PP | 542.8 | PP |
| ENSG00000123066 | THRAP2 | Hs.159799 | 142.1 | PP | 396.2 | PP |
| ENSG00000106829 | TLE4 | Hs.444213 | 309.2 | PP | 251.15 | PP |
| ENSG00000106537 | TM4SF13 | Hs.364544 | 275.4 | PP | 526.45 | PP |
| ENSG00000117289 | TXNIP | Hs.533977 | 698.8 | PP | 1099.3 | PP |
| ENSG00000119048 | UBE2B | Hs.385986 | 335 | PP | 417.45 | PP |
| ENSG00000170035 | UBE2E3 | Hs.470804 | 311.5 | PP | 258.25 | PP |
| ENSG00000132388 | UBE2G1 | Hs.462035 | 272.5 | PP | 373.45 | PP |
| ENSG00000198833 | UBE2J1 | Hs.163776 | 342.7 | PP | 357.2 | PP |
| ENSG00000114062 | UBE3A | Hs.22543 | 456.9 | PP | 470.75 | PP |
| ENSG00000009335 | UBE3C | Hs.118351 | 67.5 | PP | 175.9 | PM |
| ENSG00000153560 | UBP1 | Hs.118041 | 280.2 | PP | 408.6 | PP |
| ENSG00000170832 | USP32 | Hs.132868 | 120.2 | PP | 118.35 | PP |
| ENSG00000173218 | VANGL1 | Hs.515130 | 73.8 | PP | 89.5 | PA |
| ENSG00000144560 | VGLL4 | Hs.373959 | 299.7 | PP | 175.6 | PP |
| ENSG00000122958 | VPS26 | Hs.499925 | 508.7 | PP | 610 | PP |
| ENSG00000132716 | WDR42A | Hs.492236 | 195.9 | PP | 233.1 | PP |
| ENSG00000134308 | YWHAQ | Hs.74405 | 922.3 | PP | 2337.65 | PP |
| ENSG00000100811 | YY1 | Hs.388927 | 539.7 | PP | 740.2 | PP |
| ENSG00000066422 | ZBTB11 | Hs.301956 | 137.1 | PP | 100.65 | PP |
| ENSG00000152518 | ZFP36L2 | Hs.503093 | 4553.7 | PP | 5205.6 | PP |
| ENSG00000015171 | ZMYND11 | Hs.292265 | 403.3 | PP | 784.1 | PP |
| ENSG00000167635 | ZNF146 | Hs.301819 | 293.3 | PP | 396.15 | PP |
| ENSG00000114514 | ZNF148 | Hs.380334 | 211.6 | PP | 226.65 | PP |
| ENSG00000136451 | ZNF161 | Hs.463569 | 140.5 | PP | 193.4 | PP |
| ENSG00000179456 | ZNF238 | Hs.69997 | 235.7 | PP | 241.8 | PP |
| ENSG00000169155 | ZNF297B | Hs.355581 | 115.9 | PP | 112.4 | PA |
| ENSG00000169714 | ZNF9 | Hs.518249 | 1408.6 | PP | 1399.55 | PP |

let-7b

| | | | | | | |
|---|---|---|---|---|---|---|
| ENSG00000114770 | ABCC5 | Hs.368563 | 373.4 | PP | 322.1 | PM |
| ENSG00000184009 | ACTG1 | Hs.514581 | 11731 | PP | 14874 | PP |
| ENSG00000151320 | AKAP6 | Hs.509083 | 137.8 | PP | 117.05 | PP |
| ENSG00000145020 | AMT | Hs.102 | 345.8 | PP | 323.85 | PM |
| ENSG00000062725 | APPBP2 | Hs.84084 | 106.4 | PP | 116.15 | PA |
| ENSG00000175220 | ARHGAP1 | Hs.138860 | 594 | PP | 502.3 | PP |
| ENSG00000116539 | ASH1L | Hs.491060 | 244.1 | PP | 214.3 | PA |
| ENSG00000171456 | ASXL1 | Hs.374043 | 275.6 | PP | 307.25 | PP |
| ENSG00000082153 | BZW1 | Hs.355983 | 536.1 | PP | 794.1 | PP |
| ENSG00000139746 | C13orf10 | Hs.131715 | 119.8 | PP | 178.7 | PP |
| ENSG00000104327 | CALB1 | Hs.65425 | 79.6 | PP | 42.05 | PA |
| ENSG00000131236 | CAP1 | Hs.370581 | 765.9 | PP | 691 | PP |
| ENSG00000118971 | CCND2 | Hs.376071 | 1114.1 | PP | 1244.6 | PP |
| ENSG00000112576 | CCND3 | Hs.534307 | 539.8 | PP | 402.15 | PP |
| ENSG00000111642 | CHD4 | Hs.162233 | 299.5 | PP | 431.1 | PP |
| ENSG00000100528 | CNIH | Hs.294603 | 252.6 | PP | 391.7 | PP |
| ENSG00000009307 | CSDE1 | Hs.69855 | 1494.1 | PP | 1637.9 | PP |
| ENSG00000101266 | CSNK2A1 | Hs.446484 | 170.4 | PP | 261.4 | PP |
| ENSG00000159692 | CTBP1 | Hs.208597 | 253.9 | PP | 309.4 | PP |
| ENSG00000161202 | DVL3 | Hs.388116 | 134.8 | PP | 206.95 | PP |
| ENSG00000092847 | EIF2C1 | Hs.22867 | 292.5 | PP | 296.95 | PP |
| ENSG00000123908 | EIF2C2 | Hs.449415 | 150.2 | PP | 115.85 | PP |
| ENSG00000104131 | EIF3S1 | Hs.404056 | 340.4 | PP | 365.05 | PP |
| ENSG00000109381 | ELF2 | Hs.260903 | 150.8 | PP | 152.4 | PP |
| ENSG00000102034 | ELF4 | Hs.271940 | 309.2 | PP | 202.7 | PP |
| ENSG00000112146 | FBXO9 | Hs.216653 | 397 | PP | 447.8 | PA |
| ENSG00000100442 | FKBP3 | Hs.509226 | 386.9 | PP | 438.15 | PP |
| ENSG00000143641 | GALNT2 | Hs.300834 | 321.4 | PP | 333.55 | PP |
| ENSG00000108588 | GK001 | Hs.202011 | 160.3 | PP | 198.45 | PP |
| ENSG00000174021 | GNG5 | Hs.513557 | 683.5 | PP | 869.7 | PP |
| ENSG00000103342 | GSPT1 | Hs.528780 | 356.7 | PP | 369.6 | PP |
| ENSG00000152795 | HNRPDL | Hs.527105 | 1931.8 | PP | 1802.05 | PP |
| ENSG00000109083 | IFT20 | Hs.4187 | 322.8 | PP | 241.4 | PP |
| ENSG00000171988 | JMJD1C | Hs.413416 | 107 | PP | 198.85 | PP |
| ENSG00000100097 | LGALS1 | Hs.445351 | 454.5 | PP | 244.1 | PP |
| ENSG00000160789 | LMNA | Hs.491359 | 309.3 | PP | 352.9 | PP |
| ENSG00000111252 | LNK | Hs.506784 | 230.1 | PP | 198.8 | PP |
| ENSG00000054219 | LY75 | Hs.153563 | 126.4 | PP | 175.65 | PP |
| ENSG00000011009 | LYPLA2 /// | Hs.534075 | 585.5 | PP | 486 | PP |
| | LYPLA2P1 /// | | | | | |
| | LOC388499 | | | | | |
| ENSG00000166963 | MAP1A | Hs.194301 | 433.9 | PP | 321.4 | PM |
| ENSG00000075413 | MARK3 | Hs.35828 | 129.9 | PP | 75.05 | PP |
| ENSG00000100330 | MTMR3 | Hs.474536 | 204.7 | PP | 287 | PP |
| ENSG00000138386 | NAB1 | Hs.107474 | 187.9 | PP | 203.9 | PP |
| ENSG00000187109 | NAP1L1 | Hs.524599 | 1185.6 | PP | 1531.65 | PP |
| ENSG00000106459 | NRF1 | Hs.298069 | 157.5 | PP | 126.8 | PA |
| ENSG00000102225 | PCTK1 | Hs.496068 | 135 | PP | 88.45 | PA |
| ENSG00000184588 | PDE4B | Hs.198072 | 234.1 | PP | 349.5 | PP |
| ENSG00000136040 | PLXNC1 | Hs.551522 | 354.6 | PP | 387.1 | PP |
| ENSG00000056687 | RGL2 | Hs.509622 | 340.5 | PP | 232.1 | PA |
| ENSG00000101782 | RIOK3 | Hs.445511 | 276.1 | PP | 311.7 | PP |
| ENSG00000151967 | SCHIP1 | Hs.134665 | 497 | PP | 417.95 | PP |
| ENSG00000168758 | SEMA4C | Hs.516220 | 186.7 | PP | 157.95 | PP |
| ENSG00000102038 | SMARCA1 | Hs.152292 | 101.1 | PP | 72.95 | PA |
| ENSG00000145335 | SNCA | Hs.271771 | 337.6 | PP | 370.9 | PP |
| ENSG00000109332 | UBE2D3 | Hs.472031 | 471.2 | PP | 635.4 | PP |
| ENSG00000135655 | USP15 | Hs.434951 | 197.3 | PP | 160.35 | PP |
| ENSG00000134215 | VAV3 | Hs.267659 | 615.4 | PP | 341.3 | PP |
| ENSG00000073905 | VDAC1 | Hs.202085 | 175.6 | PP | 289.2 | PP |
| ENSG00000122958 | VPS26 | Hs.499925 | 508.7 | PP | 610 | PP |
| ENSG00000166913 | YWHAB | Hs.279920 | 222.4 | PP | 288.75 | PP |
| ENSG00000107372 | ZA20D2 | Hs.406096 | 967.2 | PP | 1262.9 | PP |

106

| | | | | | | |
|---|---|---|---|---|---|---|
| ENSG00000165029 | ABCA1 | Hs.429294 | 175.1 | PP | 156.5 | PP |
| ENSG00000136518 | ACTL6A | Hs.435326 | 84.2 | PP | 105.6 | PP |
| ENSG00000170776 | AKAP13 | Hs.459211 | 192.3 | PP | 198.75 | PA |
| ENSG00000142192 | APP | Hs.434980 | 154.6 | PP | 154.1 | PP |
| ENSG00000175220 | ARHGAP1 | Hs.138860 | 594 | PP | 502.3 | PP |
| ENSG00000117713 | ARID1A | Hs.468972 | 477.6 | PP | 444.8 | PP |
| ENSG00000111875 | ASF1A | Hs.292316 | 126.5 | PP | 114.6 | PP |
| ENSG00000143153 | ATP1B1 | Hs.291196 | 132.4 | PP | 245.55 | PP |
| ENSG00000095739 | BAMBI | Hs.533336 | 207.8 | PP | 106.85 | PP |
| ENSG00000029363 | BCLAF1 | Hs.486542 | 659.8 | PP | 1252.25 | PP |
| ENSG00000119906 | C10orf6 | Hs.447458 | 154.8 | PP | 126.8 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000143126 | CELSR2 | Hs.57652 | 215.4 | PP | 148.65 | PP |
| ENSG00000162368 | CMPK | Hs.11463 | 333.8 | PP | 336.55 | PP |
| ENSG00000111596 | CNOT2 | Hs.133350 | 431.4 | PP | 399.2 | PP |
| ENSG00000115944 | COX7A2L | Hs.339639 | 1251.1 | PP | 1258.85 | PP |
| ENSG00000167193 | CRK | Hs.461896 | 114.9 | PP | 153.9 | PP |
| ENSG00000108848 | CROP | Hs.130293 | 2166.4 | PP | 1637.55 | PP |
| ENSG00000103653 | CSK | Hs.77793 | 348.1 | PP | 217.35 | PA |
| ENSG00000102974 | CTCF | Hs.368367 | 506.1 | PP | 535.55 | PP |
| ENSG00000071626 | DAZAP1 | Hs.222510 | 49.7 | PP | 130.7 | PA |
| ENSG00000108654 | DDX5 | Hs.279806 | 3052 | PP | 3508.95 | PP |
| ENSG00000108406 | DHX40 | Hs.29403 | 233.3 | PP | 196.45 | PP |
| ENSG00000128590 | DNAJB9 | Hs.6790 | 211.5 | PP | 433.55 | PP |
| ENSG00000156508 | EEF1A1 | Hs.439552 | 40551 | PP | 40470.15 | PA |
| ENSG00000120738 | EGR1 | Hs.326035 | 128.6 | PP | 290.15 | PP |
| ENSG00000092847 | EIF2C1 | Hs.22867 | 292.5 | PP | 296.95 | PP |
| ENSG00000107581 | EIF3S10 | Hs.523299 | 522.9 | PP | 586.55 | PP |
| ENSG00000110321 | EIF4G2 | Hs.183684 | 2137 | PP | 2701.9 | PP |
| ENSG00000111145 | ELK3 | Hs.46523 | 372.1 | PP | 280.75 | PP |
| ENSG00000120616 | EPC1 | Hs.167805 | 148.9 | PP | 88.7 | PA |
| ENSG00000182944 | EWSR1 | Hs.374477 | 389.7 | PP | 457.25 | PP |
| ENSG00000106462 | EZH2 | Hs.444082 | 162.9 | PP | 216.25 | PP |
| ENSG00000139146 | FAM60A | Hs.505154 | 598.1 | PP | 788 | PP |
| ENSG00000173120 | FBXL11 | Hs.124147 | 237 | PP | 192.5 | PP |
| ENSG00000118564 | FBXL5 | Hs.479208 | 410.6 | PP | 319.15 | PP |
| ENSG00000178974 | FBXO34 | Hs.525348 | 246.4 | PP | 254.15 | PP |
| ENSG00000022267 | FHL1 | Hs.435369 | 5240 | PP | 3151.75 | PP |
| ENSG00000150907 | FOXO1A | Hs.370666 | 253.6 | PP | 140.95 | PP |
| ENSG00000143641 | GALNT2 | Hs.300834 | 321.4 | PP | 333.55 | PP |
| ENSG00000115806 | GORASP2 | Hs.431317 | 359.7 | PP | 304.85 | PP |
| ENSG00000106070 | GRB10 | Hs.164060 | 550.7 | PP | 574.15 | PP |
| ENSG00000112406 | HECA | Hs.197644 | 352.2 | PP | 377.45 | PP |
| ENSG00000100644 | HIF1A | Hs.509554 | 866.5 | PP | 1037.1 | PP |
| ENSG00000010818 | HIVEP2 | Hs.510172 | 149.6 | PP | 157.9 | PP |
| ENSG00000137361 | HLA-E | Hs.381008 | 1198.1 | PP | 595.4 | PP |
| ENSG00000029993 | HMGB3 | Hs.19114 | 309 | PP | 460.6 | PP |
| ENSG00000096746 | HNRPH3 | Hs.499891 | 408.7 | PP | 635.5 | PP |
| ENSG00000153187 | HNRPU | Hs.166463 | 470.3 | PP | 887.6 | PP |
| ENSG00000067829 | IDH3G | Hs.410197 | 249.2 | PP | 156.95 | PA |
| ENSG00000168556 | ING2 | Hs.107153 | 177 | PP | 151.85 | PP |
| ENSG00000116679 | IVNS1ABP | Hs.497183 | 251.6 | PP | 288.25 | PP |
| ENSG00000115548 | JMJD1A | Hs.531819 | 264 | PP | 207.05 | PP |
| ENSG00000102753 | KPNA3 | Hs.527919 | 250.8 | PP | 391.35 | PP |
| ENSG00000068697 | LAPTM4A | Hs.467807 | 1059.2 | PP | 1145.5 | PP |
| ENSG00000121104 | LOC81558 | Hs.514308 | 3067.5 | PP | 1370.7 | PP |
| ENSG00000163818 | LZTFL1 | Hs.30824 | 199 | PP | 253.95 | PP |
| ENSG00000173327 | MAP3K11 | Hs.502872 | 355.1 | PP | 326.8 | PP |
| ENSG00000135525 | MAP7 | Hs.486548 | 325.7 | PP | 390.1 | PP |
| ENSG00000112062 | MAPK14 | Hs.485233 | 507.4 | PP | 486.3 | PP |
| ENSG00000165406 | 8-Mar | Hs.499489 | 295.9 | PP | 295.85 | PP |
| ENSG00000038274 | MAT2B | Hs.54642 | 609.4 | PP | 461.9 | PP |
| ENSG00000015479 | MATR3 | Hs.268939 | 252.4 | PP | 382.35 | PP |
| ENSG00000152601 | MBNL1 | Hs.478000 | 294.4 | PP | 864.5 | PP |
| ENSG00000081189 | MEF2C | Hs.444409 | 426 | PP | 437.85 | PP |
| ENSG00000133606 | MKRN1 | Hs.490347 | 392.7 | PP | 350.15 | PP |
| ENSG00000100330 | MTMR3 | Hs.474536 | 204.7 | PP | 287 | PP |
| ENSG00000005810 | MYCBP2 | Hs.151411 | 187 | PP | 643.55 | PP |
| ENSG00000166579 | NDEL1 | Hs.372123 | 218.3 | PP | 187.2 | PP |
| ENSG00000082641 | NFE2L1 | Hs.514284 | 735.5 | PP | 726.35 | PP |
| ENSG00000120837 | NFYB | Hs.84928 | 204.1 | PP | 272.85 | PP |
| ENSG00000137420 | NOL7 | Hs.549161 | 1095.5 | PP | 1222.05 | PP |
| ENSG00000153234 | NR4A2 | Hs.165258 | 124.6 | PP | 242.85 | PP |
| ENSG00000124789 | NUP153 | Hs.121088 | 216.4 | PP | 291.8 | PP |
| ENSG00000046651 | OFD1 | Hs.6483 | 1022.7 | PP | 692.15 | PP |
| ENSG00000141447 | OSBPL1A | Hs.370725 | 204.6 | PP | 211.75 | PP |
| ENSG00000115421 | PAPOLG | Hs.387471 | 153.2 | PP | 91.25 | PP |
| ENSG00000157212 | PAXIP1 | Hs.443881 | 139.4 | PP | 130.7 | PP |
| ENSG00000059758 | PCTK2 | Hs.506415 | 118.3 | PP | 220.9 | PP |
| ENSG00000067057 | PFKP | Hs.26010 | 317.5 | PP | 459 | PP |
| ENSG00000025293 | PHF20 | Hs.517044 | 249.8 | PP | 173.65 | PP |
| ENSG00000171033 | PKIA | Hs.433700 | 88.6 | PP | 131.05 | PP |
| ENSG00000136040 | PLXNC1 | Hs.551522 | 354.6 | PP | 387.1 | PP |
| ENSG00000100941 | PNN | Hs.409965 | 839.5 | PP | 617.3 | PP |
| ENSG00000107758 | PPP3CB | Hs.500067 | 318.9 | PP | 241.25 | PP |
| ENSG00000154845 | PPP4R1 | Hs.464595 | 472.6 | PP | 378.85 | PP |
| ENSG00000119414 | PPP6C | Hs.495128 | 297.5 | PP | 557.55 | PP |
| ENSG00000100519 | PSMC6 | Hs.156171 | 143.7 | PP | 216.4 | PP |
| ENSG00000175354 | PTPN2 | Hs.123352 | 759.9 | PP | 273 | PP |
| ENSG00000134644 | PUM1 | Hs.281707 | 378.4 | PP | 422.3 | PP |
| ENSG00000185129 | PURA | Hs.443121 | 149.5 | PP | 224.95 | PP |
| ENSG00000130508 | PXDN | Hs.332197 | 280.3 | PP | 114.7 | PA |
| ENSG00000112531 | QKI | Hs.510324 | 284.1 | PP | 564.45 | PP |
| ENSG00000119396 | RAB14 | Hs.371563 | 140.5 | PP | 198.15 | PP |
| ENSG00000111737 | RAB35 | Hs.524788 | 394.6 | PP | 431.2 | PP |
| ENSG00000111540 | RAB5B | Hs.77690 | 234.1 | PP | 216.45 | PP |
| ENSG00000175582 | RAB6A | Hs.503222 | 371.4 | PP | 365.75 | PP |
| ENSG00000119318 | RAD23B | Hs.521640 | 156.2 | PP | 211.05 | PP |
| ENSG00000127314 | RAP1B | Hs.369920 | 1093.1 | PP | 1537.75 | PP |
| ENSG00000023287 | RB1CC1 | Hs.196102 | 95.2 | PP | 147.45 | PP |
| ENSG00000122257 | RBBP6 | Hs.188553 | 131.2 | PP | 163.35 | PP |
| ENSG00000102054 | RBBP7 | Hs.495755 | 942.6 | PP | 1356.35 | PP |
| ENSG00000127870 | RNF6 | Hs.136885 | 74.1 | PP | 122.9 | PP |
| ENSG00000167971 | RNPS1 | Hs.355643 | 1024.8 | PP | 1501.95 | PP |
| ENSG00000100784 | RPS6KA5 | Hs.510225 | 152.1 | PP | 132.75 | PP |
| ENSG00000148484 | RSU1 | Hs.524161 | 298.8 | PP | 294.55 | PP |
| ENSG00000163602 | RYBP | Hs.7910 | 398 | PP | 462.8 | PP |
| ENSG00000079332 | SARA1 | Hs.499960 | 233.7 | PP | 442.85 | PP |
| ENSG00000140497 | SCAMP2 | Hs.458917 | 411.8 | PP | 495 | PP |
| ENSG00000168385 | 2-Sep | Hs.335057 | 952.7 | PP | 1251.1 | PP |
| ENSG00000120742 | SERP1 | Hs.518326 | 285.7 | PP | 343.4 | PP |
| ENSG00000179833 | SERTAD2 | Hs.77293 | 498.4 | PP | 556.2 | PP |
| ENSG00000119335 | SET | Hs.436687 | 2967 | PP | 4307.1 | PP |
| ENSG00000116560 | SFPQ | Hs.355934 | 306.9 | PP | 689 | PP |
| ENSG00000115875 | SFRS7 | Hs.309090 | 378.7 | PP | 743.5 | PP |
| ENSG00000096717 | SIRT1 | Hs.369779 | 133.8 | PP | 124.9 | PP |
| ENSG00000113558 | SKP1A | Hs.171626 | 657.2 | PP | 969.15 | PP |
| ENSG00000059804 | SLC2A3 | Hs.419240 | 275.9 | PP | 351.5 | PP |
| ENSG00000173473 | SMARCC1 | Hs.476179 | 487 | PP | 483.35 | PP |
| ENSG00000159140 | SON | Hs.517262 | 408.8 | PP | 467.15 | PP |
| ENSG00000124766 | SOX4 | Hs.357901 | 2351.7 | PP | 2500.65 | PP |
| ENSG00000008294 | SPAG9 | Hs.463439 | 180.3 | PP | 209.9 | PP |
| ENSG00000168610 | STAT3 | Hs.463059 | 401.7 | PP | 390.05 | PP |
| ENSG00000112079 | STK38 | Hs.409578 | 365.8 | PP | 304.4 | PP |
| ENSG00000108264 | TADA2L | Hs.500066 | 108 | PP | 97.9 | PA |
| ENSG00000152291 | TGOLN2 | Hs.14894 | 430.2 | PP | 542.8 | PP |
| ENSG00000106537 | TM4SF13 | Hs.364544 | 275.4 | PP | 526.45 | PP |
| ENSG00000102804 | TSC22D1 | Hs.450972 | 1492.8 | PP | 2518.9 | PP |
| ENSG00000150991 | UBC | Hs.520348 | 2138.1 | PP | 2607.15 | PP |
| ENSG00000119048 | UBE2B | Hs.385986 | 335 | PP | 417.45 | PP |
| ENSG00000114062 | UBE3A | Hs.22543 | 456.9 | PP | 470.75 | PP |
| ENSG00000114062 | UBE3A | Hs.22543 | 126.2 | PP | 89.25 | PP |
| ENSG00000135655 | USP15 | Hs.434951 | 197.3 | PP | 160.35 | PP |
| ENSG00000173218 | VANGL1 | Hs.515130 | 73.8 | PP | 89.5 | PA |
| ENSG00000132716 | WDR42A | Hs.492236 | 195.9 | PP | 233.1 | PP |
| ENSG00000166913 | YWHAB | Hs.279920 | 222.4 | PP | 288.75 | PP |
| ENSG00000015171 | ZMYND11 | Hs.292265 | 403.3 | PP | 784.1 | PP |
| ENSG00000114514 | ZNF148 | Hs.380334 | 211.6 | PP | 226.65 | PP |
| ENSG00000179456 | ZNF238 | Hs.69997 | 235.7 | PP | 241.8 | PP |
| ENSG00000169714 | ZNF9 | Hs.518249 | 1408.6 | PP | 1399.55 | PP |

103

| | | | | | | |
|---|---|---|---|---|---|---|
| ENSG00000004455 | AK2 | Hs.470907 | 303.6 | PP | 498.6 | PP |
| ENSG00000170776 | AKAP13 | Hs.459211 | 192.3 | PP | 198.75 | PA |
| ENSG00000151320 | AKAP6 | Hs.509083 | 137.8 | PP | 117.05 | PP |
| ENSG00000117020 | AKT3 | Hs.498292 | 246.4 | PP | 259.35 | PP |
| ENSG00000142192 | APP | Hs.434980 | 154.6 | PP | 154.1 | PP |
| ENSG00000159388 | BTG2 | Hs.519162 | 588.2 | PP | 538.3 | PP |
| ENSG00000135932 | CAB39 | Hs.471593 | 527.9 | PP | 637.3 | PP |
| ENSG00000143126 | CELSR2 | Hs.57652 | 215.4 | PP | 148.65 | PP |
| ENSG00000153922 | CHD1 | Hs.519474 | 89.4 | PP | 202.95 | PP |
| ENSG00000111596 | CNOT2 | Hs.133350 | 431.4 | PP | 399.2 | PP |
| ENSG00000129083 | COPB | Hs.339278 | 686.7 | PP | 1188.55 | PP |
| ENSG00000005339 | CREBBP | Hs.459759 | 287.2 | PP | 239.6 | PP |
| ENSG00000009307 | CSDE1 | Hs.69855 | 1494.1 | PP | 1637.9 | PP |
| ENSG00000103653 | CSK | Hs.77793 | 348.1 | PP | 217.35 | PA |
| ENSG00000168036 | CTNNB1 | Hs.476018 | 343.5 | PP | 364.4 | PP |
| ENSG00000149187 | CUGBP1 | Hs.530727 | 383 | PP | 596.3 | PP |
| ENSG00000166266 | CUL5 | Hs.440320 | 285.8 | PP | 246.8 | PP |
| ENSG00000100697 | DICER1 | Hs.87889 | 176.8 | PP | 186.3 | PP |
| ENSG00000117505 | DR1 | Hs.348418 | 117.2 | PP | 188.65 | PP |
| ENSG00000104517 | EDD1 | Hs.492445 | 254.6 | PP | 227.15 | PP |
| ENSG00000092847 | EIF2C1 | Hs.22867 | 292.5 | PP | 296.95 | PP |
| ENSG00000063046 | EIF4B | Hs.512629 | 519.2 | PP | 743.85 | PP |
| ENSG00000100664 | EIF5 | Hs.433702 | 96.4 | PP | 55.8 | PA |
| ENSG00000155849 | ELMO1 | Hs.304578 | 367.9 | PP | 339.8 | PP |
| ENSG00000120616 | EPC1 | Hs.167805 | 148.9 | PP | 88.7 | PA |
| ENSG00000120699 | EXOSC8 | Hs.294041 | 196.9 | PP | 283.25 | PA |
| ENSG00000139146 | FAM60A | Hs.505154 | 598.1 | PP | 788 | PP |
| ENSG00000132589 | FLOT2 | Hs.514038 | 393.4 | PP | 355.45 | PP |
| ENSG00000104064 | GABPB2 | Hs.546852 | 129.3 | PP | 183.75 | PM |
| ENSG00000100083 | GGA1 | Hs.499158 | 669.4 | PP | 749.65 | PP |
| ENSG00000113384 | GOLPH3 | Hs.408909 | 585.8 | PP | 782.9 | PP |
| ENSG00000029993 | HMGB3 | Hs.19114 | 309 | PP | 460.6 | PP |
| ENSG00000122566 | HNRPA2B1 | Hs.487774 | 1802.2 | PP | 2268.3 | PP |
| ENSG00000152795 | HNRPDL | Hs.527105 | 1931.8 | PP | 1802.05 | PP |
| ENSG00000169045 | HNRPH1 | Hs.202166 | 1986.4 | PP | 2132.6 | PP |
| ENSG00000126945 | HNRPH2 | Hs.278857 | 189.2 | PP | 158.45 | PP |
| ENSG00000096746 | HNRPH3 | Hs.499891 | 408.7 | PP | 635.5 | PP |
| ENSG00000165119 | HNRPK | Hs.449171 | 761 | PP | 1269.35 | PP |
| ENSG00000104824 | HNRPL | Hs.446623 | 1060.2 | PP | 889.7 | PP |
| ENSG00000125944 | HNRPR | Hs.373763 | 839.4 | PP | 980.1 | PP |
| ENSG00000153187 | HNRPU | Hs.166463 | 470.3 | PP | 887.6 | PP |
| ENSG00000120087 | HOXB7 | Hs.436181 | 110.1 | PP | 108.7 | PP |
| ENSG00000173744 | HRB | Hs.443507 | 202.6 | PP | 405.7 | PP |
| ENSG00000078596 | ITM2A | Hs.17109 | 325.6 | PP | 367.7 | PP |
| ENSG00000116679 | IVNS1ABP | Hs.497183 | 251.6 | PP | 288.25 | PP |
| ENSG00000136826 | KLF4 | Hs.376206 | 536.5 | PP | 1193.7 | PP |
| ENSG00000166963 | MAP1A | Hs.194301 | 433.9 | PP | 321.4 | PM |
| ENSG00000175130 | MARCKSL1 | Hs.75061 | 314.8 | PP | 265.6 | PP |
| ENSG00000152601 | MBNL1 | Hs.478000 | 294.4 | PP | 864.5 | PP |
| ENSG00000081189 | MEF2C | Hs.444409 | 426 | PP | 437.85 | PP |
| ENSG00000125445 | MRPS7 | Hs.71787 | 381.8 | PP | 338.3 | PA |
| ENSG00000147649 | MTDH | Hs.377155 | 258.6 | PP | 329.8 | PP |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000005810 | MYCBP2 | Hs.151411 | 187 | PP | 643.55 | PP |
| ENSG00000102921 | N4BP1 | Hs.511839 | 682.3 | PP | 668.7 | PP |
| ENSG00000149308 | NPAT | Hs.171061 | 171.1 | PP | 140.65 | PP |
| ENSG00000169564 | PCBP1 | Hs.2853 | 1587.4 | PP | 1289.35 | PP |
| ENSG00000056661 | PCGF2 | Hs.371617 | 231.6 | PP | 160.8 | PP |
| ENSG00000114209 | PDCD10 | Hs.478150 | 463.6 | PP | 560.4 | PP |
| ENSG00000145675 | PIK3R1 | Hs.132225 | 249.1 | PP | 181 | PP |
| ENSG00000102024 | PLS3 | Hs.496622 | 74.1 | PP | 110.95 | PP |
| ENSG00000119414 | PPP6C | Hs.495128 | 297.5 | PP | 557.55 | PP |
| ENSG00000110958 | PTGES3 | Hs.50425 | 1018.1 | PP | 1526.7 | PP |
| ENSG00000055917 | PUM2 | Hs.467824 | 238.6 | PP | 313.25 | PP |
| ENSG00000102054 | RBBP7 | Hs.495755 | 942.6 | PP | 1356.35 | PP |
| ENSG00000144580 | RQCD1 | Hs.148767 | 284.7 | PP | 361.25 | PP |
| ENSG00000159216 | RUNX1 | Hs.149261 | 108.8 | PP | 211.2 | PP |
| ENSG00000085365 | SCAMP1 | Hs.482587 | 373.6 | PP | 392.9 | PP |
| ENSG00000151967 | SCHIP1 | Hs.134665 | 497 | PP | 417.95 | PP |
| ENSG00000137575 | SDCBP | Hs.200804 | 934 | PP | 955.25 | PP |
| ENSG00000156642 | SDFR1 | Hs.187866 | 169.4 | PP | 193.05 | PP |
| ENSG00000179833 | SERTAD2 | Hs.77293 | 498.4 | PP | 556.2 | PP |
| ENSG00000119335 | SET | Hs.436687 | 2967 | PP | 4307.1 | PP |
| ENSG00000136527 | SFRS10 | Hs.533122 | 673.5 | PP | 894.8 | PP |
| ENSG00000153914 | SFRS12 | Hs.519347 | 258 | PP | 229.6 | PP |
| ENSG00000072501 | SMC1L1 | Hs.211602 | 137.5 | PP | 329.1 | PP |
| ENSG00000163788 | SNRK | Hs.476052 | 320.6 | PP | 436.65 | PP |
| ENSG00000114520 | SNX4 | Hs.507243 | 125.1 | PP | 245.1 | PP |
| ENSG00000137642 | SORL1 | Hs.368592 | 2264.1 | PP | 1084.65 | PP |
| ENSG00000091640 | SPAG7 | Hs.90436 | 900.7 | PP | 517.9 | PP |
| ENSG00000116649 | SRM | Hs.76244 | 374.8 | PP | 439.2 | PA |
| ENSG00000178691 | SUZ12 | Hs.462732 | 602.2 | PP | 633.3 | PP |
| ENSG00000120948 | TARDBP | Hs.300624 | 735 | PP | 992.75 | PP |
| ENSG00000028839 | TBPL1 | Hs.486507 | 170.1 | PP | 187.95 | PP |
| ENSG00000154582 | TCEB1 | Hs.546305 | 511.7 | PP | 636 | PP |
| ENSG00000165169 | TCTE1L | Hs.446392 | 323 | PP | 369.75 | PP |
| ENSG00000101337 | TM9SF4 | Hs.529360 | 129.2 | PP | 121.3 | PP |
| ENSG00000076554 | TPD52 | Hs.368433 | 144.2 | PP | 223.7 | PP |
| ENSG00000166340 | TPP1 | Hs.523454 | 200.3 | PP | 229.1 | PP |
| ENSG00000143367 | TUFT1 | Hs.489922 | 173.6 | PP | 135.35 | PP |
| ENSG00000131508 | UBE2D2 | Hs.108332 | 400.1 | PP | 512.05 | PP |
| ENSG00000109332 | UBE2D3 | Hs.472031 | 471.2 | PP | 635.4 | PP |
| ENSG00000170035 | UBE2E3 | Hs.470804 | 311.5 | PP | 258.25 | PP |
| ENSG00000132388 | UBE2G1 | Hs.462035 | 272.5 | PP | 373.45 | PP |
| ENSG00000160714 | UBE2Q | Hs.516587 | 457.1 | PP | 377.95 | PP |
| ENSG00000153560 | UBP1 | Hs.118041 | 280.2 | PP | 408.6 | PP |
| ENSG00000100242 | UNC84B | Hs.517622 | 560.7 | PP | 333.25 | PP |
| ENSG90000101557 | USP14 | Hs.464416 | 542.5 | PP | 836.8 | PP |
| ENSG00000135655 | USP15 | Hs.434951 | 197.3 | PP | 160.35 | PP |
| ENSG00000108828 | VAT1 | Hs.514199 | 649.7 | PP | 585.4 | PP |
| ENSG00000035403 | VCL | Hs.500101 | 332.1 | PP | 541.1 | PP |
| ENSG00000073905 | VDAC1 | Hs.202085 | 175.6 | PP | 289.2 | PP |
| ENSG00000100568 | VTI1B | Hs.144868 | 361.4 | PP | 272.7 | PP |
| ENSG00000152518 | ZFP36L2 | Hs.503093 | 4553.7 | PP | 5205.6 | PP |
| ENSG00000010244 | ZNF207 | Hs.500775 | 918.9 | PP | 742.3 | PP |
| ENSG00000179456 | ZNF238 | Hs.69997 | 235.7 | PP | 241.8 | PP |

30b

| | | | | | | |
|---|---|---|---|---|---|---|
| ENSG00000151726 | ACSL1 | Hs.406678 | 180.6 | PP | 193.15 | PP |
| ENSG00000136518 | ACTL6A | Hs.435326 | 84.2 | PP | 105.6 | PP |
| ENSG00000072110 | ACTN1 | Hs.509765 | 212.8 | PP | 362.35 | PP |
| ENSG00000129675 | ARHGEF6 | Hs.522795 | 1207.5 | PP | 815.1 | PP |
| ENSG00000117713 | ARID1A | Hs.468972 | 477.6 | PP | 444.8 | PP |
| ENSG00000155096 | AZIN1 | Hs.459106 | 551.2 | PP | 619.2 | PP |
| ENSG00000119866 | BCL11A | Hs.370549 | 363.1 | PP | 387.8 | PP |
| ENSG00000139746 | C13orf10 | Hs.131715 | 119.8 | PP | 178.7 | PP |
| ENSG00000101084 | C20orf24 | Hs.184062 | 878.9 | PP | 1348.2 | PP |
| ENSG00000153113 | CAST | Hs.440961 | 301.7 | PP | 461.25 | PP |
| ENSG00000143126 | CELSR2 | Hs.57652 | 215.4 | PP | 148.65 | PP |
| ENSG00000060138 | CSDA | Hs.221889 | 969.1 | PP | 2074.95 | PP |
| ENSG00000103653 | CSK | Hs.77793 | 348.1 | PP | 217.35 | PA |
| ENSG00000175215 | CTDSP2 | Hs.524530 | 1247.3 | PP | 976.35 | PP |
| ENSG00000168209 | DDIT4 | Hs.523012 | 736.8 | PP | 535.85 | PP |
| ENSG00000132153 | DHX30 | Hs.517948 | 209.5 | PP | 193.5 | PP |
| ENSG00000092964 | DPYSL2 | Hs.173381 | 505.1 | PP | 495.1 | PP |
| ENSG00000142634 | EFHD2 | Hs.465374 | 292.3 | PP | 242.65 | PA |
| ENSG00000120616 | EPC1 | Hs.167805 | 148.9 | PP | 88.7 | PA |
| ENSG00000157554 | ERG | Hs.473819 | 369.4 | PP | 298.05 | PP |
| ENSG00000120705 | ETF1 | Hs.483494 | 253.8 | PP | 461.55 | PP |
| ENSG00000166147 | FBN1 | Hs.146447 | 195.2 | PP | 178.7 | PP |
| ENSG00000178974 | FBXO34 | Hs.525348 | 246.4 | PP | 254.15 | PP |
| ENSG00000109586 | GALNT7 | Hs.127407 | 97.2 | PP | 153.95 | PP |
| ENSG00000001084 | GCLC | Hs.271264 | 227.6 | PP | 219.2 | PP |
| ENSG00000152795 | HNRPDL | Hs.527105 | 1931.8 | PP | 1802.05 | PA |
| ENSG00000096746 | HNRPH3 | Hs.499891 | 408.7 | PP | 635.5 | PP |
| ENSG00000099783 | HNRPM | Hs.465808 | 968.5 | PP | 880.2 | PP |
| ENSG00000153807 | HOXA10 | Hs.110637 | 161.3 | PP | 200.7 | PA |
| ENSG00000078399 | HOXA9 | Hs.127428 | 739.2 | PP | 674 | PP |
| ENSG00000120694 | HSPH1 | Hs.36927 | 133.6 | PP | 614 | PP |
| ENSG00000136478 | HT008 | Hs.175414 | 443.2 | PP | 336.2 | PP |
| ENSG00000102753 | KPNA3 | Hs.527919 | 250.8 | PP | 391.35 | PP |
| ENSG00000065548 | LEREPO4 | Hs.368598 | 265.5 | PP | 402.5 | PP |
| ENSG00000161021 | MAML1 | Hs.484349 | 331.3 | PP | 280.35 | PP |
| ENSG00000166974 | MAPRE2 | Hs.532824 | 857.4 | PP | 715.5 | PP |
| ENSG00000168906 | MAT2A | Hs.516157 | 1011.4 | PP | 738.6 | PP |
| ENSG00000089693 | MLF2 | Hs.524214 | 247.5 | PP | 196.75 | PP |
| ENSG00000147649 | MTDH | Hs.377155 | 258.6 | PP | 329.8 | PP |
| ENSG00000005810 | MYCBP2 | Hs.151411 | 187 | PP | 643.55 | PP |
| ENSG00000166579 | NDEL1 | Hs.372123 | 218.3 | PP | 187.2 | PP |
| ENSG00000120837 | NFYB | Hs.84928 | 204.1 | PP | 272.85 | PP |
| ENSG00000153234 | NR4A2 | Hs.165258 | 124.6 | PP | 242.85 | PP |
| ENSG00000134644 | PUM1 | Hs.281707 | 378.4 | PP | 422.3 | PP |
| ENSG00000112531 | QKI | Hs.510324 | 284.1 | PP | 564.45 | PP |
| ENSG00000048991 | R3HDM | Hs.412462 | 217.6 | PP | 376.75 | PP |
| ENSG00000138069 | RAB1A | Hs.310645 | 70.8 | PP | 109.75 | PM |
| ENSG00000119318 | RAD23B | Hs.521640 | 156.2 | PP | 211.05 | PP |
| ENSG00000127314 | RAP1B | Hs.369920 | 1093.1 | PP | 1537.75 | PP |
| ENSG00000145715 | RASA1 | Hs.553501 | 138.8 | PP | 103.6 | PP |
| ENSG00000125976 | RBM12 | — | 321.1 | PP | 313.35 | PP |
| ENSG00000106615 | RHEB | Hs.283521 | 943.5 | PP | 1172.5 | PP |
| ENSG00000123091 | RNF11 | Hs.309641 | 125.3 | PP | 161.5 | PP |
| ENSG00000153574 | RPIA | Hs.469264 | 170.1 | PP | 372.1 | PP |
| ENSG00000182568 | SATB1 | Hs.517717 | 829.9 | PP | 652.05 | PP |
| ENSG00000085365 | SCAMP1 | Hs.482587 | 373.6 | PP | 392.9 | PP |
| ENSG00000121542 | SEC22L2 | Hs.477361 | 96.7 | PP | 82.45 | PA |
| ENSG00000122545 | 7-Sep | Hs.191346 | 815 | PP | 1146.5 | PP |
| ENSG00000115524 | SF3B1 | Hs.471011 | 2127.6 | PP | 2173.9 | PP |
| ENSG00000161547 | SFRS2 | Hs.73965 | 3723.5 | PP | 4390.4 | PP |
| ENSG00000059804 | SLC2A3 | Hs.419240 | 275.9 | PP | 351.5 | PP |
| ENSG00000112305 | SMAP1 | Hs.485717 | 205.2 | PP | 129.15 | PP |
| ENSG00000114520 | SNX4 | Hs.507243 | 125.1 | PP | 245.1 | PP |
| ENSG00000124766 | SOX4 | Hs.357901 | 2351.7 | PP | 2500.65 | PP |
| ENSG00000095480 | SPFH1 | Hs.150087 | 646.4 | PP | 689.15 | PP |
| ENSG00000101972 | STAG2 | Hs.496710 | 392 | PP | 375.2 | PP |
| ENSG00000178691 | SUZ12 | Hs.462732 | 602.2 | PP | 633.3 | PP |
| ENSG00000028839 | TBPL1 | Hs.486507 | 170.1 | PP | 187.95 | PP |
| ENSG00000072274 | TFRC | Hs.529618 | 1858.2 | PP | 2010.55 | PP |
| ENSG00000170348 | TMP21 | Hs.74137 | 293.5 | PP | 280.3 | PA |
| ENSG00000157514 | TSC22D3 | Hs.522074 | 1752.5 | PP | 2189.45 | PP |
| ENSG00000063244 | U2AF2 | Hs.528007 | 191 | PP | 227.15 | PP |
| ENSG00000103275 | UBE2I | Hs.302903 | 452.9 | PP | 427.25 | PP |
| ENSG00000118900 | UBN1 | Hs.440219 | 138.4 | PP | 302.15 | PP |
| ENSG00000024048 | UBR2 | Hs.529925 | 151.4 | PP | 183.45 | PP |
| ENSG00000135655 | USP15 | Hs.434951 | 197.3 | PP | 160.35 | PP |
| ENSG00000165280 | VCP | Hs.529782 | 278.5 | PP | 240.85 | PP |
| ENSG00000086666 | ZA20D3 | Hs.306329 | 735.1 | PP | 517.75 | PP |
| ENSG00000152518 | ZFP36L2 | Hs.503093 | 4553.7 | PP | 5205.6 | PP |

107

| | | | | | | |
|---|---|---|---|---|---|---|
| ENSG00000184009 | ACTG1 | Hs.514581 | 11731 | PP | 14874 | PP |
| ENSG00000137845 | ADAM10 | Hs.172028 | 221.1 | PP | 315.55 | PP |
| ENSG00000004455 | AK2 | Hs.470907 | 303.6 | PP | 498.6 | PP |
| ENSG00000170776 | AKAP13 | Hs.459211 | 192.3 | PP | 198.75 | PA |
| ENSG00000117020 | AKT3 | Hs.498292 | 246.4 | PP | 259.35 | PP |
| ENSG00000177879 | AP3S1 | Hs.406191 | 647.6 | PP | 1020.05 | PP |
| ENSG00000142192 | APP | Hs.434980 | 154.6 | PP | 154.1 | PP |
| ENSG00000134108 | ARL10C | Hs.250009 | 578.1 | PP | 591.95 | PP |
| ENSG00000136950 | ARPC5L | Hs.132499 | 512.3 | PP | 577.15 | PP |
| ENSG00000174437 | ATP2A2 | Hs.506759 | 203.5 | PP | 255.4 | PP |
| ENSG00000119866 | BCL11A | Hs.370549 | 363.1 | PP | 387.8 | PP |
| ENSG00000171791 | BCL2 | Hs.150749 | 151.5 | PP | 134.45 | PP |
| ENSG00000160058 | BSDC1 | Hs.353454 | 304.8 | PP | 151.9 | PP |
| ENSG00000159388 | BTG2 | Hs.519162 | 588.2 | PP | 538.3 | PP |
| ENSG00000168175 | C14orf32 | Hs.437831 | 559.3 | PP | 1440.6 | PP |
| ENSG00000132424 | C6orf111 | Hs.520287 | 674.8 | PP | 743.75 | PP |
| ENSG00000135932 | CAB39 | Hs.471593 | 527.9 | PP | 637.3 | PP |
| ENSG00000198898 | CAPZA2 | Hs.446123 | 100.3 | PP | 305.35 | PP |
| ENSG00000142453 | CARM1 | Hs.371416 | 141.6 | PP | 105.7 | PA |
| ENSG00000118971 | CCND2 | Hs.376071 | 1114.1 | PP | 1244.6 | PP |
| ENSG00000082258 | CCNT2 | Hs.292754 | 114.4 | PP | 102.75 | PP |
| ENSG00000135535 | CD164 | Hs.520313 | 2274.9 | PP | 2265.25 | PP |
| ENSG00000143126 | CELSR2 | Hs.57652 | 215.4 | PP | 148.65 | PP |
| ENSG00000172757 | CFL1 | Hs.170622 | 1674.1 | PP | 3264.25 | PP |
| ENSG00000111596 | CNOT2 | Hs.133350 | 431.4 | PP | 399.2 | PP |
| ENSG00000129083 | COPB | Hs.339278 | 686.7 | PP | 1188.55 | PA |
| ENSG00000166200 | COPS2 | Hs.369614 | 521.2 | PP | 700.65 | PP |
| ENSG00000005339 | CREBBP | Hs.459759 | 287.2 | PP | 239.6 | PP |
| ENSG00000099942 | CRKL | Hs.5613 | 126.4 | PP | 192.1 | PP |
| ENSG00000009307 | CSDE1 | Hs.69855 | 1494.1 | PP | 1637.9 | PP |
| ENSG00000133275 | CSNK1G2 | Hs.181390 | 529.8 | PP | 410.25 | PA |
| ENSG00000198561 | CTNND1 | Hs.166011 | 160.6 | PP | 172.65 | PM |
| ENSG00000149187 | CUGBP1 | Hs.530727 | 383 | PP | 596.3 | PP |
| ENSG00000048740 | CUGBP2 | Hs.309288 | 159.6 | PP | 334.5 | PP |
| ENSG00000166266 | CUL5 | Hs.440320 | 285.8 | PP | 246.8 | PP |
| ENSG00000085788 | DDHD2 | Hs.434966 | 351.3 | PP | 262.9 | PP |
| ENSG00000124487 | DDX3X | Hs.380774 | 769.8 | PP | 1229 | PP |
| ENSG00000139726 | DENR | Hs.22393 | 903 | PP | 1016.2 | PP |
| ENSG00000100697 | DICER1 | Hs.87889 | 176.8 | PP | 186.3 | PP |
| ENSG00000151208 | DLG5 | Hs.500245 | 73.1 | PP | 90.5 | PP |
| ENSG00000117505 | DR1 | Hs.348418 | 117.2 | PP | 188.65 | PP |
| ENSG00000112242 | E2F3 | Hs.269408 | 295.6 | PP | 585.95 | PP |
| ENSG00000104517 | EDD1 | Hs.492445 | 254.6 | PP | 227.15 | PP |
| ENSG00000092847 | EIF2C1 | Hs.22867 | 292.5 | PP | 296.95 | PP |
| ENSG00000063046 | EIF4B | Hs.512629 | 519.2 | PP | 743.85 | PP |
| ENSG00000110321 | EIF4G2 | Hs.183684 | 2137 | PP | 2701.9 | PP |
| ENSG00000100664 | EIF5 | Hs.433702 | 96.4 | PP | 55.8 | PA |
| ENSG00000155849 | ELMO1 | Hs.304578 | 367.9 | PP | 339.8 | PP |
| ENSG00000120616 | EPC1 | Hs.167805 | 148.9 | PP | 88.7 | PA |
| ENSG00000120705 | ETF1 | Hs.483494 | 253.8 | PP | 461.55 | PP |
| ENSG00000120699 | EXOSC8 | Hs.294041 | 196.9 | PP | 283.25 | PP |
| ENSG00000139146 | FAM60A | Hs.505154 | 598.1 | PP | 788 | PP |
| ENSG00000168137 | FLJ10707 | Hs.288164 | 452.5 | PP | 256.1 | PM |
| ENSG00000132589 | FLOT2 | Hs.514038 | 393.4 | PP | 355.45 | PP |
| ENSG00000125740 | FOSB | Hs.75678 | 1751.3 | PP | 3599.55 | PP |
| ENSG00000138757 | G3BP2 | Hs.303676 | 242.8 | PP | 428.55 | PP |
| ENSG00000109586 | GALNT7 | Hs.127407 | 97.2 | PP | 153.95 | PP |
| ENSG00000135677 | GNS | Hs.334534 | 261.1 | PP | 248.15 | PP |
| ENSG00000147533 | GOLGA7 | Hs.7953 | 669.8 | PP | 592.25 | PP |
| ENSG00000113384 | GOLPH3 | Hs.408909 | 585.8 | PP | 782.9 | PP |
| ENSG00000091527 | H41 | Hs.518265 | 735.1 | PP | 1129.65 | PP |
| ENSG00000180353 | HCLS1 | Hs.14601 | 464.2 | PP | 503.05 | PP |
| ENSG00000128731 | HERC2 | Hs.434890 | 257.7 | PP | 239.3 | PP |
| ENSG00000122566 | HNRPA2B1 | Hs.487774 | 1802.2 | PP | 2268.3 | PP |
| ENSG00000169045 | HNRPH1 | Hs.202166 | 1986.4 | PP | 2132.6 | PP |
| ENSG00000126945 | HNRPH2 | Hs.278857 | 189.2 | PP | 158.45 | PP |
| ENSG00000096746 | HNRPH3 | Hs.499891 | 408.7 | PP | 635.5 | PP |
| ENSG00000104824 | HNRPL | Hs.446623 | 1060.2 | PP | 889.7 | PP |
| ENSG00000125944 | HNRPR | Hs.373763 | 839.4 | PP | 980.1 | PP |
| ENSG00000153187 | HNRPU | Hs.166463 | 470.3 | PP | 887.6 | PP |
| ENSG00000153807 | HOXA10 | Hs.110637 | 161.3 | PP | 200.7 | PA |
| ENSG00000120075 | HOXB5 | Hs.149548 | 322 | PP | 238.15 | PP |
| ENSG00000120087 | HOXB7 | Hs.436181 | 110.1 | PP | 108.7 | PP |
| ENSG00000173744 | HRB | Hs.443507 | 202.6 | PP | 405.7 | PP |
| ENSG00000164164 | HSHIN1 | Hs.270851 | 303.1 | PP | 312.65 | PP |
| ENSG00000086758 | HUWE1 | Hs.136905 | 535.9 | PP | 503.6 | PP |
| ENSG00000171105 | INSR | Hs.465744 | 247.7 | PP | 292.45 | PP |
| ENSG00000078596 | ITM2A | Hs.17109 | 325.6 | PP | 367.7 | PP |
| ENSG00000116679 | IVNS1ABP | Hs.497183 | 251.6 | PP | 288.25 | PP |
| ENSG00000170852 | KBTBD2 | Hs.372541 | 412.2 | PP | 381.65 | PP |
| ENSG00000176407 | KCMF1 | Hs.345694 | 751.2 | PP | 938.3 | PP |
| ENSG00000145725 | KIAA0433 | Hs.212046 | 164.5 | PP | 186.45 | PP |
| ENSG00000136826 | KLF4 | Hs.376206 | 536.5 | PP | 1193.7 | PP |
| ENSG00000114030 | KPNA1 | Hs.161008 | 121.8 | PP | 91.95 | PP |
| ENSG00000102753 | KPNA3 | Hs.527919 | 250.8 | PP | 391.35 | PP |
| ENSG00000111885 | MAN1A1 | Hs.102788 | 309.1 | PP | 151 | PP |
| ENSG00000166963 | MAP1A | Hs.194301 | 433.9 | PP | 321.4 | PM |
| ENSG00000135341 | MAP3K7 | Hs.485968 | 180.7 | PP | 202.05 | PP |
| ENSG00000055208 | MAP3K7IP2 | Hs.269775 | 219.1 | PP | 142.8 | PP |
| ENSG00000135525 | MAP7 | Hs.486548 | 325.7 | PP | 390.1 | PP |
| ENSG00000175130 | MARCKSL1 | Hs.75061 | 314.8 | PP | 265.6 | PP |
| ENSG00000152601 | MBNL1 | Hs.478000 | 294.4 | PP | 864.5 | PP |
| ENSG00000081189 | MEF2C | Hs.444409 | 426 | PP | 437.85 | PP |
| ENSG00000198408 | MGEA5 | Hs.500842 | 363.7 | PP | 363.05 | PP |
| ENSG00000171843 | MLLT3 | Hs.493585 | 405.3 | PP | 334.5 | PP |
| ENSG00000123562 | MORF4L2 | Hs.326387 | 2189.8 | PP | 2626.1 | PP |
| ENSG00000185608 | MRPL40 | Hs.431307 | 258.6 | PP | 272.55 | PA |
| ENSG00000125445 | MRPS7 | Hs.71787 | 381.8 | PP | 338.3 | PA |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000005810 | MYCBP2 | Hs.151411 | 187 | PP | 643.55 | PP |
| ENSG00000102921 | N4BP1 | Hs.511839 | 682.3 | PP | 668.7 | PP |
| ENSG00000082641 | NFE2L1 | Hs.514284 | 735.5 | PP | 726.35 | PP |
| ENSG00000164190 | NIPBL | Hs.481927 | 165.1 | PP | 145.15 | PP |
| ENSG00000149308 | NPAT | Hs.171061 | 171.1 | PP | 140.65 | PP |
| ENSG00000180530 | NRIP1 | Hs.155017 | 842 | PP | 914.85 | PP |
| ENSG00000147162 | OGT | Hs.405410 | 165.1 | PP | 183.6 | PP |
| ENSG00000142864 | PAI-RBP1 | Hs.530412 | 337.8 | PP | 662.45 | PP |
| ENSG00000157654 | PALM2-AKAP2 | Hs.259461 | 181.4 | PP | 163.75 | PP |
| ENSG00000169564 | PCBP1 | Hs.2853 | 1587.4 | PP | 1289.35 | PP |
| ENSG00000056661 | PCGF2 | Hs.371617 | 231.6 | PP | 160.8 | PP |
| ENSG00000120265 | PCMT1 | Hs.279257 | 488.1 | PP | 649.4 | PP |
| ENSG00000059758 | PCTK2 | Hs.506415 | 118.3 | PP | 220.9 | PP |
| ENSG00000114209 | PDCD10 | Hs.478150 | 463.6 | PP | 560.4 | PP |
| ENSG00000150593 | PDCD4 | Hs.232543 | 174.3 | PP | 122.55 | PP |
| ENSG00000143870 | PDIA6 | Hs.212102 | 357.6 | PP | 852.3 | PP |
| ENSG00000025293 | PHF20 | Hs.517044 | 249.8 | PP | 173.65 | PP |
| ENSG00000145675 | PIK3R1 | Hs.132225 | 249.1 | PP | 181 | PP |
| ENSG00000137193 | PIM1 | Hs.81170 | 1021.7 | PP | 801.85 | PP |
| ENSG00000102024 | PLS3 | Hs.496622 | 74.1 | PP | 110.95 | PP |
| ENSG00000100941 | PNN | Hs.409965 | 839.5 | PP | 617.3 | PP |
| ENSG00000100614 | PPM1A | Hs.130036 | 197.3 | PP | 259.8 | PP |
| ENSG00000078304 | PPP2R5C | Hs.368264 | 174.9 | PP | 165.25 | PP |
| ENSG00000138814 | PPP3CA | Hs.435512 | 122.5 | PP | 205 | PP |
| ENSG00000119414 | PPP6C | Hs.495128 | 297.5 | PP | 557.55 | PP |
| ENSG00000110851 | PRDM4 | Hs.506655 | 188.6 | PP | 183.55 | PP |
| ENSG00000122862 | PRG1 | Hs.1908 | 802.8 | PP | 1516.65 | PP |
| ENSG00000101040 | PRKCBP1 | Hs.446240 | 305.9 | PP | 329.55 | PP |
| ENSG00000110958 | PTGES3 | Hs.50425 | 1018.1 | PP | 1526.7 | PP |
| ENSG00000055917 | PUM2 | Hs.467824 | 238.6 | PP | 313.25 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000111737 | RAB35 | Hs.524788 | 394.6 | PP | 431.2 | PP |
| ENSG00000077092 | RARB | Hs.436538 | 111.5 | PP | 156.85 | PP |
| ENSG00000122257 | RBBP6 | Hs.188553 | 131.2 | PP | 163.35 | PP |
| ENSG00000102054 | RBBP7 | Hs.495755 | 942.6 | PP | 1356.35 | PP |
| ENSG00000197676 | RBM16 | Hs.227602 | 193.8 | PP | 274 | PP |
| ENSG00000153250 | RBMS1 | Hs.470412 | 144.9 | PP | 216.05 | PP |
| ENSG00000137075 | RNF38 | Hs.333503 | 322.6 | PP | 227.7 | PP |
| ENSG00000043093 | RP42 | Hs.104613 | 236.5 | PP | 204.45 | PP |
| ENSG00000144713 | RPL32 | Hs.265174 | 21137 | PP | 15449.05 | PP |
| ENSG00000100784 | RPS6KA5 | Hs.510225 | 152.1 | PP | 132.75 | PP |
| ENSG00000144580 | RQCD1 | Hs.148767 | 284.7 | PP | 361.25 | PP |
| ENSG00000137815 | RTF1 | Hs.511096 | 194.3 | PP | 256.85 | PP |
| ENSG00000159216 | RUNX1 | Hs.149261 | 108.8 | PP | 211.2 | PP |
| ENSG00000163602 | RYBP | Hs.7910 | 398 | PP | 462.8 | PP |
| ENSG00000160633 | SAFB | Hs.23978 | 376.8 | PP | 261.35 | PP |
| ENSG00000085365 | SCAMP1 | Hs.482587 | 373.6 | PP | 392.9 | PP |
| ENSG00000121892 | SCC-112 | Hs.331431 | 220.4 | PP | 258.2 | PP |
| ENSG00000151967 | SCHIP1 | Hs.134665 | 497 | PP | 417.95 | PP |
| ENSG00000137575 | SDCBP | Hs.200804 | 934 | PP | 955.25 | PP |
| ENSG00000156642 | SDFR1 | Hs.187866 | 169.4 | PP | 193.05 | PP |
| ENSG00000138802 | SEC24B | Hs.292472 | 221.9 | PP | 223.3 | PP |
| ENSG00000125354 | 6-Sep | Hs.496666 | 335.7 | PP | 310.55 | PP |
| ENSG00000179833 | SERTAD2 | Hs.77293 | 498.4 | PP | 556.2 | PP |
| ENSG00000080546 | SESN1 | Hs.59554 | 235 | PP | 276.4 | PP |
| ENSG00000119335 | SET | Hs.436687 | 2967 | PP | 4307.1 | PP |
| ENSG00000136527 | SFRS10 | Hs.533122 | 673.5 | PP | 894.8 | PP |
| ENSG00000161547 | SFRS2 | Hs.73965 | 3723.5 | PP | 4390.4 | PP |
| ENSG00000112081 | SFRS3 | Hs.405144 | 884.9 | PP | 923.3 | PP |
| ENSG00000100650 | SFRS5 | Hs.166975 | 4459.7 | PP | 3466.15 | PP |
| ENSG00000080493 | SLC4A4 | Hs.5462 | 124.5 | PP | 268.4 | PP |
| ENSG00000073584 | SMARCE1 | Hs.463010 | 487.1 | PP | 524.6 | PP |
| ENSG00000163788 | SNRK | Hs.476052 | 320.6 | PP | 436.65 | PP |
| ENSG00000114520 | SNX4 | Hs.507243 | 125.1 | PP | 245.1 | PP |
| ENSG00000091640 | SPAG7 | Hs.90436 | 900.7 | PP | 517.9 | PP |
| ENSG00000090054 | SPTLC1 | Hs.90458 | 251.2 | PP | 312.95 | PP |
| ENSG00000116649 | SRM | Hs.76244 | 374.8 | PP | 439.2 | PA |
| ENSG00000178691 | SUZ12 | Hs.462732 | 602.2 | PP | 633.3 | PP |
| ENSG00000135090 | TAOK3 | — | 518.8 | PP | 394 | PP |
| ENSG00000120948 | TARDBP | Hs.300624 | 735 | PP | 992.75 | PP |
| ENSG00000028839 | TBPL1 | Hs.486507 | 170.1 | PP | 187.95 | PP |
| ENSG00000154582 | TCEB1 | Hs.546305 | 511.7 | PP | 636 | PP |
| ENSG00000165169 | TCTE1L | Hs.446392 | 323 | PP | 369.75 | PP |
| ENSG00000198586 | TLK1 | Hs.470586 | 150.2 | PP | 117.65 | PP |
| ENSG00000198561 | TMX2 | Hs.279861 | 484.9 | PP | 411 | PP |
| ENSG00000076554 | TPD52 | Hs.368433 | 144.2 | PP | 223.7 | PP |
| ENSG00000166340 | TPP1 | Hs.523454 | 200.3 | PP | 229.1 | PP |
| ENSG00000067167 | TRAM1 | Hs.491988 | 573.3 | PP | 894.4 | PP |
| ENSG00000166326 | TRIM44 | Hs.192103 | 340.6 | PP | 375.85 | PP |
| ENSG00000157514 | TSC22D3 | Hs.522074 | 1752.5 | PP | 2189.45 | PP |
| ENSG00000143367 | TUFT1 | Hs.489922 | 173.6 | PP | 135.35 | PP |
| ENSG00000077721 | UBE2A | Hs.379466 | 299.1 | PP | 400.85 | PP |
| ENSG00000131508 | UBE2D2 | Hs.108332 | 400.1 | PP | 512.05 | PP |
| ENSG00000109332 | UBE2D3 | Hs.472031 | 471.2 | PP | 635.4 | PP |
| ENSG00000170035 | UBE2E3 | Hs.470804 | 311.5 | PP | 258.25 | PP |
| ENSG00000160714 | UBE2Q | Hs.516587 | 457.1 | PP | 377.95 | PP |
| ENSG00000124208 | UBE2V1 /// Kua- | Hs.420529 | 504.3 | PP | 320.6 | PP |
| | UEV | | | | | |
| ENSG00000130939 | UBE4B | Hs.386404 | 291.8 | PP | 387.95 | PP |
| ENSG00000153560 | UBP1 | Hs.118041 | 280.2 | PP | 408.6 | PP |
| ENSG00000100242 | UNC84B | Hs.517622 | 560.7 | PP | 333.25 | PP |
| ENSG00000101557 | USP14 | Hs.464416 | 542.5 | PP | 836.8 | PP |
| ENSG00000135655 | USP15 | Hs.434951 | 197.3 | PP | 160.35 | PP |
| ENSG00000117533 | VAMP4 | Hs.6651 | 271.8 | PP | 299.3 | PP |
| ENSG00000108828 | VAT1 | Hs.514199 | 649.7 | PP | 585.4 | PP |
| ENSG00000035403 | VCL | Hs.500101 | 332.1 | PP | 541.1 | PP |
| ENSG00000069329 | VPS35 | Hs.447547 | 705.1 | PP | 966.5 | PP |
| ENSG00000100568 | VTI1B | Hs.144868 | 361.4 | PP | 272.7 | PP |
| ENSG00000084463 | WBP11 | Hs.524281 | 245.3 | PP | 325.4 | PP |
| ENSG00000085433 | WDR47 | Hs.546347 | 90.4 | PP | 116.6 | PP |
| ENSG00000109685 | WHSC1 | Hs.113876 | 239.5 | PP | 283.1 | PP |
| ENSG00000157954 | WIPI-2 | Hs.122363 | 680.5 | PP | 572.7 | PP |
| ENSG00000157954 | WIPI-2 | Hs.122363 | 265.4 | PP | 363.1 | PP |
| ENSG00000083896 | YT521 | Hs.175955 | 318.2 | PP | 505.9 | PP |
| ENSG00000128245 | YWHAH | Hs.226755 | 195 | PP | 195.4 | PP |
| ENSG00000134308 | YWHAQ | Hs.74405 | 922.3 | PP | 2337.65 | PP |
| ENSG00000152518 | ZFP36L2 | Hs.503093 | 4553.7 | PP | 5205.6 | PP |
| ENSG00000010244 | ZNF207 | Hs.500775 | 918.9 | PP | 742.3 | PP |
| ENSG00000179456 | ZNF238 | Hs.69997 | 235.7 | PP | 241.8 | PP |

30c

| | | | | | | |
|---|---|---|---|---|---|---|
| ENSG00000114770 | ABCC5 | Hs.368563 | 373.4 | PP | 322.1 | PM |
| ENSG00000136518 | ACTL6A | Hs.435326 | 84.2 | PP | 105.6 | PP |
| ENSG00000072110 | ACTN1 | Hs.509765 | 212.8 | PP | 362.35 | PP |
| ENSG00000117713 | ARID1A | Hs.468972 | 477.6 | PP | 444.8 | PP |
| ENSG00000119866 | BCL11A | Hs.370549 | 363.1 | PP | 387.8 | PP |
| ENSG00000139746 | C13orf10 | Hs.131715 | 119.8 | PP | 178.7 | PP |
| ENSG00000116489 | CAPZA1 | Hs.514934 | 766.7 | PP | 1165.8 | PP |
| ENSG00000163660 | CCNL1 | Hs.4859 | 1082.6 | PP | 1052.5 | PP |
| ENSG00000070831 | CDC42 | Hs.467637 | 620.1 | PP | 437.75 | PP |
| ENSG00000005339 | CREBBP | Hs.459759 | 287.2 | PP | 239.6 | PP |
| ENSG00000060138 | CSDA | Hs.221889 | 969.1 | PP | 2074.95 | PP |
| ENSG00000103653 | CSK | Hs.77793 | 348.1 | PP | 217.35 | PA |
| ENSG00000175215 | CTDSP2 | Hs.524530 | 1247.3 | PP | 976.35 | PP |
| ENSG00000008283 | CYB561 | Hs.355264 | 72.3 | PP | 201.35 | PP |
| ENSG00000168209 | DDIT4 | Hs.523012 | 736.8 | PP | 535.85 | PP |
| ENSG00000151208 | DLG5 | Hs.500245 | 73.1 | PP | 90.5 | PP |
| ENSG00000092847 | EIF2C1 | Hs.22867 | 292.5 | PP | 296.95 | PP |
| ENSG00000120616 | EPC1 | Hs.167805 | 148.9 | PP | 88.7 | PA |
| ENSG00000120705 | ETF1 | Hs.483494 | 253.8 | PP | 461.55 | PP |
| ENSG00000166147 | FBN1 | Hs.146447 | 195.2 | PP | 178.7 | PP |
| ENSG00000109586 | GALNT7 | Hs.127407 | 97.2 | PP | 153.95 | PP |
| ENSG00000001084 | GCLC | Hs.271264 | 227.6 | PP | 219.2 | PP |
| ENSG00000170144 | HNRPA3 | Hs.516539 | 375.5 | PP | 558.6 | PP |
| ENSG00000152795 | HNRPDL | Hs.527105 | 1931.8 | PP | 1802.05 | PP |
| ENSG00000099783 | HNRPM | Hs.465808 | 968.5 | PP | 880.2 | PP |
| ENSG00000153807 | HOXA10 | Hs.110637 | 161.3 | PP | 200.7 | PA |
| ENSG00000074356 | HSA277841 | Hs.120963 | 222.9 | PP | 150.05 | PP |
| ENSG00000144381 | HSPD1 | Hs.113684 | 2868.6 | PP | 5689.3 | PP |
| ENSG00000155090 | KLF10 | Hs.435001 | 348.9 | PP | 357.65 | PP |
| ENSG00000102753 | KPNA3 | Hs.527919 | 250.8 | PP | 391.35 | PP |
| ENSG00000065548 | LEREPO4 | Hs.368598 | 265.5 | PP | 402.5 | PP |
| ENSG00000148943 | LIN7C | Hs.91393 | 87.3 | PP | 121.85 | PP |
| ENSG00000161021 | MAML1 | Hs.484349 | 331.3 | PP | 280.35 | PP |
| ENSG00000166974 | MAPRE2 | Hs.532824 | 857.4 | PP | 715.5 | PP |
| ENSG00000168906 | MAT2A | Hs.516157 | 1011.4 | PP | 738.6 | PP |
| ENSG00000089693 | MLF2 | Hs.524214 | 247.5 | PP | 196.75 | PP |
| ENSG00000147649 | MTDH | Hs.377155 | 258.6 | PP | 329.8 | PP |
| ENSG00000005810 | MYCBP2 | Hs.151411 | 187 | PP | 643.55 | PP |
| ENSG00000120837 | NFYB | Hs.84928 | 204.1 | PP | 272.85 | PP |
| ENSG00000147162 | OGT | Hs.405410 | 165.1 | PP | 183.6 | PP |
| ENSG00000136040 | PLXNC1 | Hs.551522 | 354.6 | PP | 387.1 | PP |
| ENSG00000112245 | PTP4A1 | Hs.227777 | 439.5 | PP | 431.2 | PP |
| ENSG00000134644 | PUM1 | Hs.281707 | 378.4 | PP | 422.3 | PP |
| ENSG00000112531 | QKI | Hs.510324 | 284.1 | PP | 564.45 | PP |
| ENSG00000048991 | R3HDM | Hs.412462 | 217.6 | PP | 376.75 | PP |
| ENSG00000138069 | RAB1A | Hs.310645 | 70.8 | PP | 109.75 | PM |
| ENSG00000075785 | RAB7 | Hs.15738 | 99.2 | PP | 107.25 | PP |
| ENSG00000119318 | RAD23B | Hs.521640 | 156.2 | PP | 211.05 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000125976 | RBM12 | — | 321.1 | PP | 313.35 | PP |
| ENSG00000106615 | RHEB | Hs.283521 | 943.5 | PP | 1172.5 | PP |
| ENSG00000123091 | RNF11 | Hs.309641 | 125.3 | PP | 161.5 | PP |
| ENSG00000153574 | RPIA | Hs.469264 | 170.1 | PP | 372.1 | PP |
| ENSG00000163602 | RYBP | Hs.7910 | 398 | PP | 462.8 | PP |
| ENSG00000182568 | SATB1 | Hs.517717 | 829.9 | PP | 652.05 | PP |
| ENSG00000168758 | SEMA4C | Hs.516220 | 186.7 | PP | 157.95 | PP |
| ENSG00000122545 | 7-Sep | Hs.191346 | 815 | PP | 1146.5 | PP |
| ENSG00000136450 | SFRS1 | Hs.68714 | 1264.9 | PP | 1622.65 | PP |
| ENSG00000161547 | SFRS2 | Hs.73965 | 3723.5 | PP | 4390.4 | PP |
| ENSG00000115875 | SFRS7 | Hs.309090 | 378.7 | PP | 743.5 | PP |
| ENSG00000096717 | SIRT1 | Hs.369779 | 133.8 | PP | 124.9 | PP |
| ENSG00000059804 | SLC2A3 | Hs.419240 | 275.9 | PP | 351.5 | PP |
| ENSG00000112305 | SMAP1 | Hs.485717 | 205.2 | PP | 129.15 | PP |
| ENSG00000114520 | SNX4 | Hs.507243 | 125.1 | PP | 245.1 | PP |
| ENSG00000124766 | SOX4 | Hs.357901 | 2351.7 | PP | 2500.65 | PP |
| ENSG00000095480 | SPFH1 | Hs.150087 | 646.4 | PP | 689.15 | PP |
| ENSG00000101972 | STAG2 | Hs.496710 | 392 | PP | 375.2 | PP |
| ENSG00000178691 | SUZ12 | Hs.462732 | 602.2 | PP | 633.3 | PP |
| ENSG00000120948 | TARDBP | Hs.300624 | 735 | PP | 992.75 | PP |
| ENSG00000100207 | TCF20 | Hs.475018 | 251.3 | PP | 281.15 | PP |
| ENSG00000144043 | TEX261 | Hs.516087 | 360.9 | PP | 229.45 | PA |
| ENSG00000072274 | TFRC | Hs.529618 | 1858.2 | PP | 2010.55 | PP |
| ENSG00000170348 | TMP21 | Hs.74137 | 293.5 | PP | 280.3 | PA |
| ENSG00000183864 | TOB2 | Hs.474978 | 165.3 | PP | 196.4 | PP |
| ENSG00000157514 | TSC22D3 | Hs.522074 | 1752.5 | PP | 2189.45 | PP |
| ENSG00000063244 | U2AF2 | Hs.528007 | 191 | PP | 227.15 | PP |
| ENSG00000103275 | UBE2I | Hs.302903 | 452.9 | PP | 427.25 | PP |
| ENSG00000009335 | UBE3C | Hs.118351 | 67.5 | PP | 175.9 | PM |
| ENSG00000118900 | UBN1 | Hs.440219 | 138.4 | PP | 302.15 | PP |
| ENSG00000135655 | USP15 | Hs.434951 | 197.3 | PP | 160.35 | PP |
| ENSG00000107372 | ZA20D2 | Hs.406096 | 967.2 | PP | 1262.9 | PP |
| ENSG00000086666 | ZA20D3 | Hs.306329 | 735.1 | PP | 517.75 | PP |

93

| | | | | | | |
|---|---|---|---|---|---|---|
| ENSG00000111780 | 15E1.2 | Hs.549299 | 68.7 | PP | 68.6 | PP |
| ENSG00000165029 | ABCA1 | Hs.429294 | 175.1 | PP | 156.5 | PP |
| ENSG00000117020 | AKT3 | Hs.498292 | 246.4 | PP | 259.35 | PP |
| ENSG00000142192 | APP | Hs.434980 | 154.6 | PP | 154.1 | PP |
| ENSG00000137962 | ARHGAP29 | Hs.483238 | 72.2 | PP | 65.3 | PP |
| ENSG00000104880 | ARHGEF18 | Hs.465763 | 366 | PP | 254.9 | PA |
| ENSG00000054267 | ARID4B | Hs.533633 | 227 | PP | 252.45 | PP |
| ENSG00000111875 | ASF1A | Hs.292316 | 126.5 | PP | 114.6 | PP |
| ENSG00000143970 | ASXL2 | Hs.119815 | 694.8 | PP | 535.25 | PP |
| ENSG00000017260 | ATP2C1 | Hs.546361 | 229.1 | PP | 322.9 | PP |
| ENSG00000095739 | BAMBI | Hs.533336 | 207.8 | PP | 106.85 | PP |
| ENSG00000119866 | BCL11A | Hs.370549 | 363.1 | PP | 387.8 | PP |
| ENSG00000060762 | BRP44L | Hs.172755 | 387.7 | PP | 334.3 | PP |
| ENSG00000133639 | BTG1 | Hs.255935 | 955.9 | PP | 1057.95 | PP |
| ENSG00000110075 | C11orf23 | Hs.503022 | 402.6 | PP | 319.5 | PA |
| ENSG00000152242 | C18orf25 | Hs.116486 | 129.7 | PP | 130.6 | PA |
| ENSG00000141452 | C18orf8 | Hs.529006 | 232.8 | PP | 242.05 | PP |
| ENSG00000110888 | C1QDC1 | Hs.234355 | 291.3 | PP | 261.05 | PP |
| ENSG00000118971 | CCND2 | Hs.376071 | 1114.1 | PP | 1244.6 | PP |
| ENSG00000163660 | CCNL1 | Hs.4859 | 1082.6 | PP | 1052.5 | PP |
| ENSG00000082258 | CCNT2 | Hs.292754 | 114.4 | PP | 102.75 | PP |
| ENSG00000155111 | CDC2L6 | Hs.193251 | 325 | PP | 341.25 | PP |
| ENSG00000123374 | CDK2 | Hs.19192 | 171.5 | PP | 336.45 | PP |
| ENSG00000177200 | CHD9 | Hs.530936 | 222.6 | PP | 263 | PP |
| ENSG00000198791 | CNOT7 | Hs.170553 | 250.3 | PP | 508.5 | PP |
| ENSG00000138663 | COPS4 | Hs.190384 | 331.1 | PP | 393.8 | PA |
| ENSG00000167193 | CRK | Hs.461896 | 114.9 | PP | 153.9 | PP |
| ENSG00000102974 | CTCF | Hs.368367 | 506.1 | PP | 535.55 | PP |
| ENSG00000048740 | CUGBP2 | Hs.309288 | 159.6 | PP | 334.5 | PP |
| ENSG00000121966 | CXCR4 | Hs.421986 | 532.4 | PP | 1919.4 | PP |
| ENSG00000183283 | DAZAP2 | Hs.369761 | 1267.2 | PP | 976.7 | PP |
| ENSG00000124487 | DDX3X | Hs.380774 | 769.8 | PP | 1229 | PP |
| ENSG00000108406 | DHX40 | Hs.29403 | 233.3 | PP | 196.45 | PP |
| ENSG00000151208 | DLG5 | Hs.500245 | 73.1 | PP | 90.5 | PP |
| ENSG00000105993 | DNAJB6 | Hs.490745 | 189.7 | PP | 348.5 | PP |
| ENSG00000074603 | DPP8 | Hs.458609 | 250.4 | PP | 288 | PP |
| ENSG00000092964 | DPYSL2 | Hs.173381 | 505.1 | PP | 495.1 | PP |
| ENSG00000112242 | E2F3 | Hs.269408 | 295.6 | PP | 585.95 | PP |
| ENSG00000104517 | EDD1 | Hs.492445 | 254.6 | PP | 227.15 | PP |
| ENSG00000156508 | EEF1A1 | Hs.439552 | 40551 | PP | 40470.15 | PP |
| ENSG00000149547 | EI24 | Hs.343911 | 166.1 | PP | 305.55 | PP |
| ENSG00000092847 | EIF2C1 | Hs.22867 | 292.5 | PP | 296.95 | PP |
| ENSG00000104131 | EIF3S1 | Hs.404056 | 340.4 | PP | 365.05 | PP |
| ENSG00000110321 | EIF4G2 | Hs.183684 | 2137 | PP | 2701.9 | PP |
| ENSG00000111145 | ELK3 | Hs.46523 | 372.1 | PP | 280.75 | PP |
| ENSG00000062598 | ELMO2 | Hs.210469 | 70.6 | PP | 59.8 | PP |
| ENSG00000050405 | EPLIN | Hs.525419 | 265.5 | PP | 309.7 | PP |
| ENSG00000139146 | FAM60A | Hs.505154 | 598.1 | PP | 788 | PP |
| ENSG00000173120 | FBXL11 | Hs.124147 | 237 | PP | 192.5 | PP |
| ENSG00000118564 | FBXL5 | Hs.479208 | 410.6 | PP | 319.15 | PP |
| ENSG00000138081 | FBXO11 | Hs.553547 | 155.7 | PP | 259.35 | PP |
| ENSG00000022267 | FHL1 | Hs.435369 | 5240 | PP | 3151.75 | PP |
| ENSG00000168137 | FLJ10707 | Hs.288164 | 452.5 | PP | 256.1 | PM |
| ENSG00000111011 | FLJ11021 | Hs.432996 | 202 | PP | 392.6 | PP |
| ENSG00000137273 | FOXF2 | Hs.484423 | 109.9 | PP | 89.85 | PP |
| ENSG00000198815 | FOXJ3 | Hs.26023 | 458.7 | PP | 380.45 | PP |
| ENSG00000196700 | GM632 | Hs.551552 | 103.5 | PP | 145.05 | PP |
| ENSG00000135677 | GNS | Hs.334534 | 261.1 | PP | 248.15 | PP |
| ENSG00000112406 | HECA | Hs.197644 | 352.2 | PP | 377.45 | PP |
| ENSG00000010818 | HIVEP2 | Hs.510172 | 149.6 | PP | 157.9 | PP |
| ENSG00000122566 | HNRPA2B1 | Hs.487774 | 1802.2 | PP | 2268.3 | PP |
| ENSG00000096746 | HNRPH3 | Hs.499891 | 408.7 | PP | 635.5 | PP |
| ENSG00000105323 | HNRPUL1 | Hs.155218 | 406.9 | PP | 401.15 | PP |
| ENSG00000143164 | IQWD1 | Hs.435741 | 168.5 | PP | 156.4 | PP |
| ENSG00000115548 | JMJD1A | Hs.531819 | 264 | PP | 207.05 | PP |
| ENSG00000177606 | JUN | Hs.525704 | 1300.5 | PP | 2286.95 | PP |
| ENSG00000170852 | KBTBD2 | Hs.372541 | 412.2 | PP | 381.65 | PP |
| ENSG00000114030 | KPNA1 | Hs.161008 | 121.8 | PP | 91.95 | PP |
| ENSG00000005893 | LAMP2 | Hs.496684 | 251.3 | PP | 179 | PP |
| ENSG00000068697 | LAPTM4A | Hs.467807 | 1059.2 | PP | 1145.5 | PP |
| ENSG00000107929 | LARP5 | Hs.498455 | 274.8 | PP | 203.9 | PP |
| ENSG00000172292 | LASS6 | Hs.506829 | 536.2 | PP | 463.8 | PP |
| ENSG00000121104 | LOC81558 | Hs.514308 | 3067.5 | PP | 1370.7 | PP |
| ENSG00000123684 | LPGAT1 | Hs.497674 | 106 | PP | 114.3 | PP |
| ENSG00000146963 | LUC7L2 | Hs.530118 | 153.5 | PP | 227.3 | PP |
| ENSG00000163818 | LZTFL1 | Hs.30824 | 199 | PP | 253.95 | PP |
| ENSG00000121579 | MAK3 | Hs.269528 | 268.8 | PP | 307 | PP |
| ENSG00000161021 | MAML1 | Hs.484349 | 331.3 | PP | 280.35 | PP |
| ENSG00000173327 | MAP3K11 | Hs.502872 | 355.1 | PP | 326.8 | PP |
| ENSG00000197442 | MAP3K5 | Hs.186486 | 227.6 | PP | 272.25 | PP |
| ENSG00000104814 | MAP4K1 | Hs.95424 | 586.5 | PP | 231.45 | PP |
| ENSG00000135525 | MAP7 | Hs.486548 | 325.7 | PP | 390.1 | PP |
| ENSG00000101367 | MAPRE1 | Hs.472437 | 196.1 | PP | 206.85 | PM |
| ENSG00000165406 | 8-Mar | Hs.499489 | 295.9 | PP | 295.85 | PP |
| ENSG00000038274 | MAT2B | Hs.54642 | 609.4 | PP | 461.9 | PP |
| ENSG00000081189 | MEF2C | Hs.444409 | 426 | PP | 437.85 | PP |
| ENSG00000133858 | MGC23401 | Hs.527874 | 291 | PP | 309.55 | PP |
| ENSG00000198408 | MGEA5 | Hs.500842 | 363.7 | PP | 363.05 | PP |
| ENSG00000133606 | MKRN1 | Hs.490347 | 392.7 | PP | 350.15 | PP |
| ENSG00000100330 | MTMR3 | Hs.474536 | 204.7 | PP | 287 | PP |
| ENSG00000108389 | MTMR4 | Hs.514373 | 330.7 | PP | 407.2 | PP |
| ENSG00000158747 | NBL1 | Hs.439671 | 370.3 | PP | 360.6 | PA |
| ENSG00000125356 | NDUFA1 | Hs.534168 | 2121.1 | PP | 1598.7 | PP |
| ENSG00000149308 | NPAT | Hs.171061 | 171.1 | PP | 140.65 | PP |
| ENSG00000153234 | NR4A2 | Hs.165258 | 124.6 | PP | 242.85 | PP |
| ENSG00000147162 | OGT | Hs.405410 | 165.1 | PP | 183.6 | PP |
| ENSG00000141447 | OSBPL1A | Hs.370725 | 204.6 | PP | 211.75 | PP |
| ENSG00000091039 | OSBPL8 | Hs.430849 | 212 | PP | 251.65 | PP |
| ENSG00000090060 | PAPOLA | Hs.253726 | 307.4 | PP | 286 | PP |
| ENSG00000185619 | PCGF3 | Hs.144309 | 192.1 | PP | 237.55 | PP |
| ENSG00000025293 | PHF20 | Hs.517044 | 249.8 | PP | 173.65 | PP |
| ENSG00000171033 | PKIA | Hs.433700 | 88.6 | PP | 131.05 | PP |
| ENSG00000136040 | PLXNC1 | Hs.551522 | 354.6 | PP | 387.1 | PP |
| ENSG00000119414 | PPP6C | Hs.495128 | 297.5 | PP | 557.55 | PP |
| ENSG00000110851 | PRDM4 | Hs.506655 | 188.6 | PP | 183.55 | PP |
| ENSG00000171862 | PTEN | Hs.500466 | 353.7 | PP | 334.4 | PP |
| ENSG00000112531 | QKI | Hs.510324 | 284.1 | PP | 564.45 | PP |
| ENSG00000103769 | RAB11A | Hs.321541 | 1087.7 | PP | 1020.15 | PP |
| ENSG00000119396 | RAB14 | Hs.371563 | 140.5 | PP | 198.15 | PP |
| ENSG00000111737 | RAB35 | Hs.524788 | 394.6 | PP | 431.2 | PP |
| ENSG00000111540 | RAB5B | Hs.77690 | 234.1 | PP | 216.45 | PP |
| ENSG00000175582 | RAB6A | Hs.503222 | 371.4 | PP | 365.75 | PP |
| ENSG00000119318 | RAD23B | Hs.521640 | 156.2 | PP | 211.05 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000101265 | RASSF2 | Hs.379970 | 129.4 | PP | 203.9 | PP |
| ENSG00000023287 | RB1CC1 | Hs.196102 | 95.2 | PP | 147.45 | PP |
| ENSG00000102054 | RBBP7 | Hs.495755 | 942.6 | PP | 1356.35 | PP |
| ENSG00000103479 | RBL2 | Hs.513609 | 203.5 | PP | 188.25 | PP |
| ENSG00000127870 | RNF6 | Hs.136885 | 74.1 | PP | 122.9 | PP |
| ENSG00000043093 | RP42 | Hs.104613 | 236.5 | PP | 204.45 | PP |
| ENSG00000117676 | RPS6KA1 | Hs.149957 | 288.1 | PP | 176.7 | PA |
| ENSG00000100784 | RPS6KA5 | Hs.510225 | 152.1 | PP | 132.75 | PP |
| ENSG00000159216 | RUNX1 | Hs.149261 | 108.8 | PP | 211.2 | PP |
| ENSG00000198838 | RYR3 | Hs.369250 | 116.8 | PP | 131.8 | PP |
| ENSG00000079332 | SARA1 | Hs.499960 | 233.7 | PP | 442.85 | PP |
| ENSG00000140497 | SCAMP2 | Hs.458917 | 411.8 | PP | 495 | PP |
| ENSG00000075223 | SEMA3C | Hs.269109 | 95.2 | PP | 98.7 | PP |
| ENSG00000168385 | 2-Sep | Hs.335057 | 952.7 | PP | 1251.1 | PP |
| ENSG00000179833 | SERTAD2 | Hs.77293 | 498.4 | PP | 556.2 | PP |
| ENSG00000161547 | SFRS2 | Hs.73965 | 3723.5 | PP | 4390.4 | PP |
| ENSG00000162105 | SHANK2 | Hs.268726 | 137.1 | PP | 128.55 | PP |
| ENSG00000175387 | SMAD2 | Hs.12253 | 243.4 | PP | 321.4 | PP |
| ENSG00000163788 | SNRK | Hs.476052 | 320.6 | PP | 436.65 | PP |
| ENSG00000159140 | SON | Hs.517262 | 408.8 | PP | 467.15 | PP |
| ENSG00000196700 | SOX18 | Hs.8619 | 1264.6 | PP | 2674 | PP |
| ENSG00000172845 | SP3 | Hs.531587 | 137.8 | PP | 293.35 | PP |
| ENSG00000008294 | SPAG9 | Hs.463439 | 180.3 | PP | 209.9 | PP |
| ENSG00000112079 | STK38 | Hs.409578 | 365.8 | PP | 304.4 | PP |
| ENSG00000101945 | SUV39H1 | Hs.522639 | 267.8 | PP | 383.35 | PP |
| ENSG00000135090 | TAOK3 | — | 518.8 | PP | 394 | PP |
| ENSG00000120948 | TARDBP | Hs.300624 | 735 | PP | 992.75 | PP |
| ENSG00000106052 | TAX1BP1 | Hs.34576 | 550 | PP | 486.2 | PP |
| ENSG00000163659 | TIPARP | Hs.12813 | 567.1 | PP | 1548.4 | PP |
| ENSG00000183864 | TOB2 | Hs.474978 | 165.3 | PP | 196.4 | PP |
| ENSG00000166340 | TPP1 | Hs.523454 | 200.3 | PP | 229.1 | PP |
| ENSG00000117289 | TXNIP | Hs.533977 | 698.8 | PP | 1099.3 | PP |
| ENSG00000119048 | UBE2B | Hs.385986 | 335 | PP | 417.45 | PP |
| ENSG00000198833 | UBE2J1 | Hs.163776 | 342.7 | PP | 357.2 | PP |
| ENSG00000114062 | UBE3A | Hs.22543 | 456.9 | PP | 470.75 | PP |
| ENSG00000135655 | USP15 | Hs.434951 | 197.3 | PP | 160.35 | PP |
| ENSG00000173218 | VANGL1 | Hs.515130 | 73.8 | PP | 89.5 | PA |
| ENSG00000122958 | VPS26 | Hs.499925 | 508.7 | PP | 610 | PP |
| ENSG00000164924 | YWHAZ | Hs.492407 | 363.6 | PP | 520.8 | PP |
| ENSG00000066422 | ZBTB11 | Hs.301956 | 137.1 | PP | 100.65 | PP |
| ENSG00000186908 | ZDHHC17 | Hs.4014 | 123.5 | PP | 166.2 | PP |
| ENSG00000152518 | ZFP36L2 | Hs.503093 | 4553.7 | PP | 5205.6 | PP |
| ENSG00000015171 | ZMYND11 | Hs.292265 | 403.3 | PP | 784.1 | PP |
| ENSG00000114514 | ZNF148 | Hs.380334 | 211.6 | PP | 226.65 | PP |
| ENSG00000179456 | ZNF238 | Hs.69997 | 235.7 | PP | 241.8 | PP |
| ENSG00000169155 | ZNF297B | Hs.355581 | 115.9 | PP | 112.4 | PA |

16b

| | | | | | | |
|---|---|---|---|---|---|---|
| ENSG00000114770 | ABCC5 | Hs.368563 | 373.4 | PP | 322.1 | PM |
| ENSG00000161533 | ACOX1 | Hs.464137 | 46.2 | PP | 73.15 | PP |
| ENSG00000151726 | ACSL1 | Hs.406678 | 180.6 | PP | 193.15 | PP |
| ENSG00000138071 | ACTR2 | Hs.393201 | 343.5 | PP | 1110.1 | PP |
| ENSG00000023516 | AKAP11 | Hs.105105 | 88.5 | PP | 69.7 | PP |
| ENSG00000151320 | AKAP6 | Hs.509083 | 137.8 | PP | 117.05 | PP |
| ENSG00000117020 | AKT3 | Hs.498292 | 246.4 | PP | 259.35 | PP |
| ENSG00000107104 | ANKRD15 | Hs.493272 | 235.2 | PP | 131.45 | PP |
| ENSG00000142192 | APP | Hs.434980 | 154.6 | PP | 154.1 | PP |
| ENSG00000095139 | ARCN1 | Hs.33642 | 285 | PP | 317.05 | PP |
| ENSG00000168374 | ARF4 | Hs.148330 | 129 | PP | 157.5 | PP |
| ENSG00000134108 | ARL10C | Hs.250009 | 578.1 | PP | 591.95 | PP |
| ENSG00000110025 | ARL2 | Hs.502836 | 357.6 | PP | 283.6 | PA |
| ENSG00000147416 | ATP6V1B2 | Hs.295917 | 179.5 | PP | 183.85 | PP |
| ENSG00000128524 | ATP6V1F | Hs.78089 | 1146.8 | PP | 588.65 | PM |
| ENSG00000171791 | BCL2 | Hs.150749 | 151.5 | PP | 134.45 | PP |
| ENSG00000138756 | BMP2K | Hs.146551 | 239.6 | PP | 167.25 | PP |
| ENSG00000145741 | BTF3 | Hs.529798 | 6371.5 | PP | 4884.45 | PP |
| ENSG00000159388 | BTG2 | Hs.519162 | 588.2 | PP | 538.3 | PP |
| ENSG00000110075 | C11orf23 | Hs.503022 | 402.6 | PP | 319.5 | PA |
| ENSG00000168175 | C14orf32 | Hs.437831 | 559.3 | PP | 1440.6 | PP |
| ENSG00000031003 | C5orf5 | Hs.483473 | 256.3 | PP | 316.85 | PP |
| ENSG00000132424 | C6orf111 | Hs.520287 | 674.8 | PP | 743.75 | PP |
| ENSG00000135932 | CAB39 | Hs.471593 | 527.9 | PP | 637.3 | PP |
| ENSG00000135837 | CAP350 | Hs.413045 | 177.4 | PP | 220.9 | PP |
| ENSG00000198898 | CAPZA2 | Hs.446123 | 100.3 | PP | 305.35 | PA |
| ENSG00000067955 | CBFB | Hs.460988 | 719.1 | PP | 1032 | PP |
| ENSG00000118971 | CCND2 | Hs.376071 | 1114.1 | PP | 1244.6 | PP |
| ENSG00000082258 | CCNT2 | Hs.292754 | 114.4 | PP | 102.75 | PP |
| ENSG00000135535 | CD164 | Hs.520313 | 2274.9 | PP | 2265.25 | PP |
| ENSG00000004897 | CDC27 | Hs.463295 | 217.6 | PP | 220.6 | PP |
| ENSG00000070831 | CDC42 | Hs.467637 | 620.1 | PP | 437.75 | PP |
| ENSG00000170779 | CDCA4 | Hs.34045 | 204.6 | PP | 276.95 | PA |
| ENSG00000143126 | CELSR2 | Hs.57652 | 215.4 | PP | 148.65 | PP |
| ENSG00000153922 | CHD1 | Hs.519474 | 89.4 | PP | 202.95 | PP |
| ENSG00000177200 | CHD9 | Hs.530936 | 222.6 | PP | 263 | PP |
| ENSG00000110172 | CHORDC1 | Hs.22857 | 84.8 | PP | 156.6 | PP |
| ENSG00000111596 | CNOT2 | Hs.133350 | 431.4 | PP | 399.2 | PP |
| ENSG00000166200 | COPS2 | Hs.369614 | 521.2 | PP | 700.65 | PP |
| ENSG00000005339 | CREBBP | Hs.459759 | 287.2 | PP | 239.6 | PP |
| ENSG00000111269 | CREBL2 | Hs.13313 | 353.2 | PP | 216.25 | PP |
| ENSG00000099942 | CRKL | Hs.5613 | 126.4 | PP | 192.1 | PP |
| ENSG00000009307 | CSDE1 | Hs.69855 | 1494.1 | PP | 1637.9 | PP |
| ENSG00000036257 | CUL3 | Hs.372286 | 554.5 | PP | 473.25 | PP |
| ENSG00000166266 | CUL5 | Hs.440320 | 285.8 | PP | 246.8 | PP |
| ENSG00000121966 | CXCR4 | Hs.421986 | 532.4 | PP | 1919.4 | PP |
| ENSG00000165659 | DACH1 | Hs.129452 | 95.1 | PP | 144.8 | PP |
| ENSG00000124487 | DDX3X | Hs.380774 | 769.8 | PP | 1229 | PP |
| ENSG00000067048 | DDX3Y | Hs.99120 | 302.6 | PP | 309.6 | PA |
| ENSG00000158796 | DEDD | Hs.517342 | 203.3 | PP | 276 | PP |
| ENSG00000139726 | DENR | Hs.22393 | 903 | PP | 1016.2 | PP |
| ENSG00000082458 | DLG3 | Hs.522680 | 136.2 | PP | 89.45 | PP |
| ENSG00000077380 | DNCI2 | Hs.546250 | 424.1 | PP | 451.65 | PP |
| ENSG00000112242 | E2F3 | Hs.269408 | 295.6 | PP | 585.95 | PP |
| ENSG00000171570 | EGLN2 | Hs.515417 | 259 | PP | 194.45 | PM |
| ENSG00000092847 | EIF2C1 | Hs.22867 | 292.5 | PP | 296.95 | PP |
| ENSG00000104131 | EIF3S1 | Hs.404056 | 340.4 | PP | 365.05 | PP |
| ENSG00000107581 | EIF3S10 | Hs.523299 | 522.9 | PP | 586.55 | PP |
| ENSG00000156976 | EIF4A2 | Hs.478553 | 3569 | PP | 6526.55 | PP |
| ENSG00000063046 | EIF4B | Hs.512629 | 519.2 | PP | 743.85 | PP |
| ENSG00000151247 | EIF4E | Hs.249718 | 156.7 | PP | 224 | PP |
| ENSG00000110321 | EIF4G2 | Hs.183684 | 2137 | PP | 2701.9 | PP |
| ENSG00000158417 | EIF5B | Hs.158688 | 101.4 | PP | 113.8 | PP |
| ENSG00000143420 | ENSA | Hs.510087 | 977.6 | PP | 1356.35 | PP |
| ENSG00000108799 | EZH1 | Hs.194669 | 211.4 | PP | 194.45 | PP |
| ENSG00000139146 | FAM60A | Hs.505154 | 598.1 | PP | 788 | PP |
| ENSG00000088832 | FKBP1A | Hs.471933 | 373.2 | PP | 520.25 | PP |
| ENSG00000138757 | G3BP2 | Hs.303676 | 242.8 | PP | 428.55 | PP |
| ENSG00000141429 | GALNT1 | Hs.514806 | 220 | PP | 273.8 | PP |
| ENSG00000065135 | GNAI3 | Hs.73799 | 603.4 | PP | 763.55 | PP |
| ENSG00000110768 | GTF2H1 | Hs.523510 | 167.7 | PP | 191.7 | PP |
| ENSG00000112339 | HBS1L | Hs.378532 | 268.3 | PP | 299.85 | PP |
| ENSG00000172534 | HCFC1 | Hs.83634 | 146.2 | PP | 233.9 | PA |
| ENSG00000143321 | HDGF | Hs.506748 | 243.6 | PP | 858.8 | PP |
| ENSG00000181061 | HIG1 | Hs.7917 | 396.7 | PP | 564.55 | PP |
| ENSG00000078140 | HIP2 | Hs.50308 | 60.5 | PP | 68.4 | PP |
| ENSG00000122566 | HNRPA2B1 | Hs.487774 | 1802.2 | PP | 2268.3 | PP |
| ENSG00000170144 | HNRPA3 | Hs.516539 | 375.5 | PP | 558.6 | PP |
| ENSG00000126945 | HNRPH2 | Hs.278857 | 189.2 | PP | 158.45 | PP |
| ENSG00000104824 | HNRPL | Hs.446623 | 1060.2 | PP | 889.7 | PP |
| ENSG00000153807 | HOXA10 | Hs.110637 | 161.3 | PP | 200.7 | PA |
| ENSG00000120087 | HOXB7 | Hs.436181 | 110.1 | PP | 108.7 | PP |
| ENSG00000164164 | HSHIN1 | Hs.270851 | 303.1 | PP | 312.65 | PP |
| ENSG00000109971 | HSPA8 | Hs.180414 | 2288 | PP | 5615.25 | PP |
| ENSG00000171105 | INSR | Hs.465744 | 247.7 | PP | 292.45 | PP |
| ENSG00000143164 | IQWD1 | Hs.435741 | 168.5 | PP | 156.4 | PP |
| ENSG00000008083 | JARID2 | Hs.269059 | 1015 | PP | 894 | PP |
| ENSG00000170852 | KBTBD2 | Hs.372541 | 412.2 | PP | 381.65 | PP |
| ENSG00000176407 | KCMF1 | Hs.345694 | 751.2 | PP | 938.3 | PP |
| ENSG00000121774 | KHDRBS1 | Hs.445893 | 1363 | PP | 1395.2 | PP |
| ENSG00000131149 | KIAA0182 | Hs.461647 | 273.2 | PP | 316.15 | PP |
| ENSG00000126775 | KIAA0831 | Hs.414809 | 254.4 | PP | 217.45 | PP |
| ENSG00000136826 | KLF4 | Hs.376206 | 536.5 | PP | 1193.7 | PP |
| ENSG00000102753 | KPNA3 | Hs.527919 | 250.8 | PP | 391.35 | PP |
| ENSG00000197017 | LAT1-3TM | Hs.444600 | 269.6 | PP | 389.55 | PP |
| | /// BOLA2 | | | | | |
| | /// | | | | | |
| | LOC595101 | | | | | |
| ENSG00000168016 | LBA1 | Hs.517761 | 235.1 | PP | 174.9 | PP |
| ENSG00000120963 | LOC51123 | Hs.374485 | 636.6 | PP | 626.2 | PP |
| ENSG00000198799 | LRIG2 | Hs.448972 | 194.9 | PP | 178.65 | PP |
| ENSG00000011009 | LYPLA2 /// | Hs.534075 | 585.5 | PP | 486 | PP |
| | LYPLA2P1 | | | | | |
| | /// | | | | | |
| | LOC388499 | | | | | |
| ENSG00000127603 | MACF1 | Hs.472475 | 222.8 | PP | 248.35 | PP |
| ENSG00000169032 | MAP2K1 | Hs.145442 | 188.8 | PP | 162.95 | PP |
| ENSG00000047849 | MAP4 | Hs.517949 | 302.6 | PP | 276.5 | PP |
| ENSG00000135525 | MAP7 | Hs.486548 | 325.7 | PP | 390.1 | PP |
| ENSG00000101367 | MAPRE1 | Hs.472437 | 196.1 | PP | 206.85 | PM |
| ENSG00000152601 | MBNL1 | Hs.478000 | 294.4 | PP | 864.5 | PP |
| ENSG00000198408 | MGEA5 | Hs.500842 | 363.7 | PP | 363.05 | PP |
| ENSG00000051825 | MPHOSPH9 | Hs.507175 | 261.2 | PP | 160.5 | PP |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000136504 | MYST2 | Hs.21907 | 331.7 | PP | 192.25 | PP |
| ENSG00000102921 | N4BP1 | Hs.511839 | 682.3 | PP | 668.7 | PP |
| ENSG00000082641 | NFE2L1 | Hs.514284 | 735.5 | PP | 726.35 | PP |
| ENSG00000153234 | NR4A2 | Hs.165258 | 124.6 | PP | 242.85 | PP |
| ENSG00000180530 | NRIP1 | Hs.155017 | 842 | PP | 914.85 | PP |
| ENSG00000147162 | OGT | Hs.405410 | 165.1 | PP | 183.6 | PP |
| ENSG00000167770 | OTUB1 | Hs.473788 | 266 | PP | 155.35 | PA |
| ENSG00000142864 | PAI-RBP1 | Hs.530412 | 337.8 | PP | 662.45 | PP |
| ENSG00000145730 | PAM | Hs.369430 | 187.3 | PP | 178.45 | PP |
| ENSG00000168283 | PCGF4 | Hs.380403 | 254.7 | PP | 304.1 | PP |
| ENSG00000120265 | PCMT1 | Hs.279257 | 488.1 | PP | 649.4 | PP |
| ENSG00000081154 | PCNP | Hs.549185 | 281.6 | PP | 507.95 | PP |
| ENSG00000059758 | PCTK2 | Hs.506415 | 118.3 | PP | 220.9 | PP |
| ENSG00000150593 | PDCD4 | Hs.232543 | 174.3 | PP | 122.55 | PP |
| ENSG00000143870 | PDIA6 | Hs.212102 | 357.6 | PP | 852.3 | PP |
| ENSG00000130024 | PHF10 | Hs.435933 | 375.2 | PP | 371.15 | PP |
| ENSG00000043143 | PHF15 | Hs.483419 | 234 | PP | 320.3 | PP |
| ENSG00000025293 | PHF20 | Hs.517044 | 249.8 | PP | 173.65 | PP |
| ENSG00000033800 | PIAS1 | Hs.162458 | 102 | PP | 168.6 | PP |
| ENSG00000145675 | PIK3R1 | Hs.132225 | 249.1 | PP | 181 | PP |
| ENSG00000137193 | PIM1 | Hs.81170 | 1021.7 | PP | 801.85 | PP |
| ENSG00000118495 | PLAGL1 | Hs.444975 | 187.1 | PP | 357.8 | PP |
| ENSG00000052126 | PLEKHA5 | Hs.188614 | 312.4 | PP | 557.7 | PP |
| ENSG00000125686 | PPARBP | Hs.462956 | 187.8 | PP | 321.7 | PP |
| ENSG00000100614 | PPM1A | Hs.130036 | 197.3 | PP | 259.8 | PP |
| ENSG00000163806 | PPP1CB | Hs.468018 | 65.7 | PP | 135.05 | PP |
| ENSG00000112498 | PPP1R11 | Hs.82887 | 484.7 | PP | 463.3 | PP |
| ENSG00000078304 | PPP2R5C | Hs.368264 | 174.9 | PP | 165.25 | PP |
| ENSG00000107758 | PPP3CB | Hs.500067 | 318.9 | PP | 241.25 | PP |
| ENSG00000119414 | PPP6C | Hs.495128 | 297.5 | PP | 557.55 | PP |
| ENSG00000110851 | PRDM4 | Hs.506655 | 188.6 | PP | 183.55 | PP |
| ENSG00000122862 | PRG1 | Hs.1908 | 802.8 | PP | 1516.65 | PP |
| ENSG00000117569 | PTBP2 | Hs.269895 | 183 | PP | 160.15 | PP |
| ENSG00000134644 | PUM1 | Hs.281707 | 378.4 | PP | 422.3 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000138069 | RAB1A | Hs.310645 | 70.8 | PP | 109.75 | PM |
| ENSG00000119318 | RAD23B | Hs.521640 | 156.2 | PP | 211.05 | PP |
| ENSG00000010017 | RANBP9 | Hs.306242 | 381.3 | PP | 489.95 | PP |
| ENSG00000127314 | RAP1B | Hs.369920 | 1093.1 | PP | 1537.75 | PP |
| ENSG00000138698 | RAP1GDS1 | Hs.132858 | 169 | PP | 213.85 | PP |
| ENSG00000109756 | RAPGEF2 | Hs.113912 | 394.9 | PP | 551.95 | PP |
| ENSG00000077092 | RARB | Hs.436538 | 111.5 | PP | 156.85 | PP |
| ENSG00000197676 | RBM16 | Hs.227602 | 193.8 | PP | 274 | PP |
| ENSG00000004534 | RBM6 | Hs.188879 | 383.9 | PP | 251.5 | PP |
| ENSG00000067560 | RHOA | Hs.247077 | 1537.7 | PP | 2368.8 | PP |
| ENSG00000126858 | RHOT1 | Hs.462742 | 196.4 | PP | 304.85 | PP |
| ENSG00000176624 | RKHD2 | Hs.465144 | 210.8 | PP | 216 | PP |
| ENSG00000022840 | RNF10 | Hs.442798 | 327.5 | PP | 282.35 | PM |
| ENSG00000134758 | RNF138 | Hs.302408 | 186.9 | PP | 511.9 | PP |
| ENSG00000137075 | RNF38 | Hs.333503 | 322.6 | PP | 227.7 | PP |
| ENSG00000167971 | RNPS1 | Hs.355643 | 1024.8 | PP | 1501.95 | PP |
| ENSG00000144713 | RPL32 | Hs.265174 | 21137 | PP | 15449.05 | PP |
| ENSG00000130779 | RSN | Hs.524809 | 76.6 | PP | 58.05 | PP |
| ENSG00000137815 | RTF1 | Hs.511096 | 194.3 | PP | 256.85 | PP |
| ENSG00000115310 | RTN4 | Hs.429581 | 710.8 | PP | 1341.5 | PP |
| ENSG00000159216 | RUNX1 | Hs.149261 | 108.8 | PP | 211.2 | PP |
| ENSG00000163602 | RYBP | Hs.7910 | 398 | PP | 462.8 | PP |
| ENSG00000130066 | SAT | Hs.28491 | 972.4 | PP | 1889.05 | PP |
| ENSG00000151967 | SCHIP1 | Hs.134665 | 497 | PP | 417.95 | PP |
| ENSG00000010803 | SCMH1 | Hs.87464 | 251.2 | PP | 190.45 | PP |
| ENSG00000137575 | SDCBP | Hs.200804 | 934 | PP | 955.25 | PP |
| ENSG00000156642 | SDFR1 | Hs.187866 | 169.4 | PP | 193.05 | PP |
| ENSG00000092421 | SEMA6A | Hs.156967 | 140 | PP | 182.2 | PP |
| ENSG00000168385 | 2-Sep | Hs.335057 | 952.7 | PP | 1251.1 | PP |
| ENSG00000080546 | SESN1 | Hs.59554 | 235 | PP | 276.4 | PP |
| ENSG00000116754 | SFRS11 | Hs.479693 | 819.9 | PP | 1077.55 | PP |
| ENSG00000153914 | SFRS12 | Hs.519347 | 258 | PP | 229.6 | PP |
| ENSG00000161547 | SFRS2 | Hs.73965 | 3723.5 | PP | 4390.4 | PP |
| ENSG00000100650 | SFRS5 | Hs.166975 | 4459.7 | PP | 3466.15 | PP |
| ENSG00000162105 | SHANK2 | Hs.268726 | 137.1 | PP | 128.55 | PP |
| ENSG00000108061 | SHOC2 | Hs.104315 | 283.3 | PP | 262.55 | PP |
| ENSG00000196470 | SIAH1 | Hs.295923 | 216.3 | PP | 346.15 | PP |
| ENSG00000096717 | SIRT1 | Hs.369779 | 133.8 | PP | 124.9 | PP |
| ENSG00000059804 | SLC2A3 | Hs.419240 | 275.9 | PP | 351.5 | PP |
| ENSG00000080493 | SLC4A4 | Hs.5462 | 124.5 | PP | 268.4 | PP |
| ENSG00000112305 | SMAP1 | Hs.485717 | 205.2 | PP | 129.15 | PP |
| ENSG00000102038 | SMARCA1 | Hs.152292 | 101.1 | PP | 72.95 | PA |
| ENSG00000163788 | SNRK | Hs.476052 | 320.6 | PP | 436.65 | PP |
| ENSG00000120833 | SOCS2 | Hs.485572 | 715.8 | PP | 806.65 | PP |
| ENSG00000159140 | SON | Hs.517262 | 408.8 | PP | 467.15 | PP |
| ENSG00000137642 | SORL1 | Hs.368592 | 2264.1 | PP | 1084.65 | PP |
| ENSG00000147475 | SPFH2 | Hs.125849 | 125.1 | PP | 180.9 | PP |
| ENSG00000090054 | SPTLC1 | Hs.90458 | 251.2 | PP | 312.95 | PP |
| ENSG00000096063 | SRPK1 | Hs.443861 | 231.6 | PP | 200.5 | PP |
| ENSG00000124783 | SSR1 | Hs.114033 | 600.7 | PP | 686.65 | PP |
| ENSG00000167323 | STIM1 | Hs.501735 | 143.2 | PP | 99.7 | PP |
| ENSG00000166301 | STK19 | Hs.485102 | 527.6 | PP | 424 | PP |
| ENSG00000117632 | STMN1 | Hs.209983 | 546.6 | PP | 663.4 | PP |
| ENSG00000092201 | SUPT16H | Hs.213724 | 531.6 | PP | 508.3 | PP |
| ENSG00000178691 | SUZ12 | Hs.462732 | 602.2 | PP | 633.3 | PP |
| ENSG00000008282 | SYPL1 | Hs.80919 | 157.3 | PP | 190.55 | PP |
| ENSG00000135090 | TAOK3 | — | 518.8 | PP | 394 | PP |
| ENSG00000120948 | TARDBP | Hs.300624 | 735 | PP | 992.75 | PP |
| ENSG00000112592 | TBP | Hs.1100 | 218 | PP | 214.95 | PP |
| ENSG00000028839 | TBPL1 | Hs.486507 | 170.1 | PP | 187.95 | PP |
| ENSG00000165169 | TCTE1L | Hs.446392 | 323 | PP | 369.75 | PP |
| ENSG00000198586 | TLK1 | Hs.470586 | 150.2 | PP | 117.65 | PP |
| ENSG00000170348 | TMP21 | Hs.74137 | 293.5 | PP | 280.3 | PA |
| ENSG00000076554 | TPD52 | Hs.368433 | 144.2 | PP | 223.7 | PP |
| ENSG00000111669 | TPI1 | Hs.524219 | 1154.9 | PP | 1213.85 | PP |
| ENSG00000067167 | TRAM1 | Hs.491988 | 573.3 | PP | 894.4 | PP |
| ENSG00000054116 | TRAPPC3 | Hs.523131 | 245.5 | PP | 340.85 | PM |
| ENSG00000157514 | TSC22D3 | Hs.522074 | 1752.5 | PP | 2189.45 | PP |
| ENSG00000077721 | UBE2A | Hs.379466 | 299.1 | PP | 400.85 | PP |
| ENSG00000131508 | UBE2D2 | Hs.108332 | 400.1 | PP | 512.05 | PP |
| ENSG00000170142 | UBE2E1 | Hs.164853 | 318.4 | PP | 488.3 | PP |
| ENSG00000170035 | UBE2E3 | Hs.470804 | 311.5 | PP | 258.25 | PP |
| ENSG00000103275 | UBE2I | Hs.302903 | 452.9 | PP | 427.25 | PP |
| ENSG00000198833 | UBE2J1 | Hs.163776 | 342.7 | PP | 357.2 | PP |
| ENSG00000160714 | UBE2Q | Hs.516587 | 457.1 | PP | 377.95 | PP |
| ENSG00000124208 | UBE2V1 /// | Hs.420529 | 504.3 | PP | 320.6 | PP |
| | Kua-UEV | | | | | |
| ENSG00000130939 | UBE4B | Hs.386404 | 291.8 | PP | 387.95 | PP |
| ENSG00000152484 | USP12 | Hs.42400 | 170.4 | PP | 158.75 | PP |
| ENSG00000101557 | USP14 | Hs.464416 | 542.5 | PP | 836.8 | PP |
| ENSG00000135655 | USP15 | Hs.434951 | 197.3 | PP | 160.35 | PP |
| ENSG00000170832 | USP32 | Hs.132868 | 120.2 | PP | 118.35 | PP |
| ENSG00000049245 | VAMP3 | Hs.66708 | 730.1 | PP | 588.5 | PP |
| ENSG00000118640 | VAMP8 | Hs.534373 | 891.4 | PP | 1443.1 | PP |
| ENSG00000101558 | VAPA | Hs.165195 | 1051.8 | PP | 1198.45 | PP |
| ENSG00000108828 | VAT1 | Hs.514199 | 649.7 | PP | 585.4 | PP |
| ENSG00000167987 | VPS37C | Hs.523715 | 163.6 | PP | 123.15 | PA |
| ENSG00000143952 | VPS54 | Hs.48499 | 187.9 | PP | 151.5 | PP |
| ENSG00000100568 | VTI1B | Hs.144868 | 361.4 | PP | 272.7 | PP |
| ENSG00000084463 | WBP11 | Hs.524281 | 245.3 | PP | 325.4 | PP |
| ENSG00000085433 | WDR47 | Hs.546347 | 90.4 | PP | 116.6 | PP |
| ENSG00000157954 | WIPI-2 | Hs.122363 | 680.5 | PP | 572.7 | PP |
| ENSG00000109046 | WSB1 | Hs.446017 | 679.3 | PP | 880.75 | PP |
| ENSG00000115254 | XAB1 | Hs.18259 | 544.2 | PP | 566.2 | PP |
| ENSG00000083896 | YT521 | Hs.175955 | 318.2 | PP | 505.9 | PP |
| ENSG00000108953 | YWHAE | Hs.513851 | 328.6 | PP | 424.95 | PP |
| ENSG00000128245 | YWHAH | Hs.226755 | 195 | PP | 195.4 | PP |
| ENSG00000134308 | YWHAQ | Hs.74405 | 922.3 | PP | 2337.65 | PP |
| ENSG00000107372 | ZA20D2 | Hs.406096 | 967.2 | PP | 1262.9 | PP |
| ENSG00000086666 | ZA20D3 | Hs.306329 | 735.1 | PP | 517.75 | PP |
| ENSG00000186908 | ZDHHC17 | Hs.4014 | 123.5 | PP | 166.2 | PP |
| ENSG00000179456 | ZNF238 | Hs.69997 | 235.7 | PP | 241.8 | PP |
| ENSG00000149182 | ZNF289 | Hs.436204 | 600.4 | PP | 330.95 | PP |
| ENSG00000119725 | ZNF410 | Hs.270869 | 164.4 | PP | 257.1 | PP |
| ENSG00000036549 | ZZZ3 | Hs.480506 | 125 | PP | 189.7 | PP |

10a

| | | | | | | |
|---|---|---|---|---|---|---|
| ENSG00000165029 | ABCA1 | Hs.429294 | 175.1 | PP | 156.5 | PP |
| ENSG00000184009 | ACTG1 | Hs.514581 | 11731 | PP | 14874 | PP |
| ENSG00000172493 | AFF1 | Hs.480190 | 480.6 | PP | 776.35 | PP |
| ENSG00000062725 | APPBP2 | Hs.84084 | 106.4 | PP | 116.15 | PA |
| ENSG00000177479 | ARIH2 | Hs.241558 | 296.7 | PP | 352.5 | PP |
| ENSG00000171456 | ASXL1 | Hs.374043 | 275.6 | PP | 307.25 | PP |
| ENSG00000095739 | BAMBI | Hs.533336 | 207.8 | PP | 106.85 | PP |
| ENSG00000163444 | C1orf37 | Hs.497443 | 290.5 | PP | 291.25 | PP |
| ENSG00000136932 | C9orf156 | Hs.9196 | 133.4 | PP | 159.1 | PP |
| ENSG00000111596 | CNOT2 | Hs.133350 | 431.4 | PP | 399.2 | PP |
| ENSG00000005339 | CREBBP | Hs.459759 | 287.2 | PP | 239.6 | PP |
| ENSG00000167193 | CRK | Hs.461896 | 114.9 | PP | 153.9 | PP |
| ENSG00000060138 | CSDA | Hs.221889 | 969.1 | PP | 2074.95 | PP |
| ENSG00000048740 | CUGBP2 | Hs.309288 | 159.6 | PP | 334.5 | PP |
| ENSG00000036257 | CUL3 | Hs.372286 | 554.5 | PP | 473.25 | PP |
| ENSG00000071626 | DAZAP1 | Hs.222510 | 49.7 | PP | 130.7 | PA |
| ENSG00000124487 | DDX3X | Hs.380774 | 769.8 | PP | 1229 | PP |
| ENSG00000161202 | DVL3 | Hs.388116 | 134.8 | PP | 206.95 | PP |
| ENSG00000112242 | E2F3 | Hs.269408 | 295.6 | PP | 585.95 | PP |
| ENSG00000170522 | ELOVL6 | Hs.412939 | 96.5 | PP | 156.45 | PP |
| ENSG00000168137 | FLJ10707 | Hs.288164 | 452.5 | PP | 256.1 | PM |
| ENSG00000141429 | GALNT1 | Hs.514806 | 220 | PP | 273.8 | PP |
| ENSG00000107485 | GATA3 | Hs.524134 | 170 | PP | 187.35 | PP |
| ENSG00000159592 | GPBP1L1 | Hs.238432 | 275.7 | PP | 300.05 | PP |
| ENSG00000106070 | GRB10 | Hs.164060 | 550.7 | PP | 574.15 | PP |
| ENSG00000110768 | GTF2H1 | Hs.523510 | 167.7 | PP | 191.7 | PA |
| ENSG00000077809 | GTF2I /// | Hs.520459 | 325.7 | PP | 556 | PP |
| | GTF2IP1 | | | | | |
| ENSG00000103657 | HERC1 | Hs.210385 | 186.9 | PP | 236.05 | PP |
| ENSG00000010818 | HIVEP2 | Hs.510172 | 149.6 | PP | 157.9 | PP |
| ENSG00000138668 | HNRPD | Hs.480073 | 785.7 | PP | 1067.15 | PP |
| ENSG00000165119 | HNRPK | Hs.449171 | 761 | PP | 1269.35 | PP |
| ENSG00000126814 | KIAA1393 | Hs.380159 | 1228.6 | PP | 1731.35 | PP |
| ENSG00000136826 | KLF4 | Hs.376206 | 536.5 | PP | 1193.7 | PP |
| ENSG00000120963 | LOC51123 | Hs.374485 | 636.6 | PP | 626.2 | PP |
| ENSG00000124831 | LRRFIP1 | Hs.471779 | 429.8 | PP | 408.25 | PP |
| ENSG00000135341 | MAP3K7 | Hs.485968 | 180.7 | PP | 202.05 | PP |
| ENSG00000047849 | MAP4 | Hs.517949 | 302.6 | PP | 276.5 | PP |
| ENSG00000101367 | MAPRE1 | Hs.472437 | 196.1 | PP | 206.85 | PM |
| ENSG00000168906 | MAT2A | Hs.516157 | 1011.4 | PP | 738.6 | PP |
| ENSG00000134046 | MBD2 | Hs.25674 | 271.8 | PP | 193.25 | PP |
| ENSG00000125445 | MRPS7 | Hs.71787 | 381.8 | PP | 338.3 | PA |
| ENSG00000100330 | MTMR3 | Hs.474536 | 204.7 | PP | 287 | PP |
| ENSG00000198646 | NCOA6 | Hs.368971 | 334.7 | PP | 445.5 | PP |
| ENSG00000196498 | NCOR2 | Hs.137510 | 340.6 | PP | 374.05 | PP |
| ENSG00000147140 | NONO | Hs.533282 | 2434.4 | PP | 2382.1 | PP |
| ENSG00000125686 | PPARBP | Hs.462956 | 187.8 | PP | 321.7 | PP |
| ENSG00000078304 | PPP2R5C | Hs.368264 | 174.9 | PP | 165.25 | PP |
| ENSG00000171862 | PTEN | Hs.500466 | 353.7 | PP | 334.4 | PP |
| ENSG00000134644 | PUM1 | Hs.281707 | 378.4 | PP | 422.3 | PP |
| ENSG00000055917 | PUM2 | Hs.467824 | 238.6 | PP | 313.25 | PP |
| ENSG00000109756 | RAPGEF2 | Hs.113912 | 394.9 | PP | 551.95 | PP |
| ENSG00000023287 | RB1CC1 | Hs.196102 | 95.2 | PP | 147.45 | PP |
| ENSG00000137075 | RNF38 | Hs.333503 | 322.6 | PP | 227.7 | PP |
| ENSG00000163602 | RYBP | Hs.7910 | 398 | PP | 462.8 | PP |
| ENSG00000138760 | SCARB2 | Hs.349656 | 81.4 | PP | 116.15 | PP |
| ENSG00000136450 | SFRS1 | Hs.68714 | 1264.9 | PP | 1622.65 | PP |
| ENSG00000134294 | SLC38A2 | Hs.221847 | 634.3 | PP | 1026.55 | PP |
| ENSG00000112305 | SMAP1 | Hs.485717 | 205.2 | PP | 129.15 | PP |
| ENSG00000173473 | SMARCC1 | Hs.476179 | 487 | PP | 483.35 | PP |
| ENSG00000114520 | SNX4 | Hs.507243 | 125.1 | PP | 245.1 | PP |
| ENSG00000159140 | SON | Hs.517262 | 408.8 | PP | 467.15 | PP |
| ENSG00000008294 | SPAG9 | Hs.463439 | 180.3 | PP | 209.9 | PP |
| ENSG00000113140 | SPARC | Hs.111779 | 427 | PP | 408.4 | PP |
| ENSG00000102572 | STK24 | Hs.508514 | 958.4 | PP | 1363.75 | PP |
| ENSG00000143367 | TUFT1 | Hs.489922 | 173.6 | PP | 135.35 | PP |
| ENSG00000109332 | UBE2D3 | Hs.472031 | 471.2 | PP | 635.4 | PP |
| ENSG00000132388 | UBE2G1 | Hs.462035 | 272.5 | PP | 373.45 | PP |
| ENSG00000015171 | ZMYND11 | Hs.292265 | 403.3 | PP | 784.1 | PP |

16a

see 16b

181a

| Ensemble Gene | | | PBSC | |
|---|---|---|---|---|
| ID | Gene Symbol | Unigene | RFU | BM RFU |
| ENSG00000114770 | ABCC5 | Hs.368563 | 373.4 | 322.1 |
| ENSG00000151726 | ACSL1 | Hs.406678 | 180.6 | 193.15 |
| ENSG00000173020 | ADRBK1 | Hs.83636 | 706 | 549.3 |
| ENSG00000172493 | AFF1 | Hs.480190 | 480.6 | 776.35 |
| ENSG00000168448 | AGPAT1 | Hs.409230 | 228.5 | 163.65 |
| ENSG00000151320 | AKAP6 | Hs.509083 | 137.8 | 117.05 |
| ENSG00000117020 | AKT3 | Hs.498292 | 246.4 | 259.35 |
| ENSG00000107104 | ANKRD15 | Hs.493272 | 235.2 | 131.45 |
| ENSG00000084234 | APLP2 | Hs.370247 | 1126.7 | 668.55 |
| ENSG00000062725 | APPBP2 | Hs.84084 | 106.4 | 116.15 |
| ENSG00000134287 | ARF3 | Hs.119177 | 302.8 | 475.75 |
| ENSG00000177479 | ARIH2 | Hs.241558 | 296.7 | 352.5 |
| ENSG00000138175 | ARL3 | Hs.182215 | 285.9 | 225.1 |
| ENSG00000143153 | ATP1B1 | Hs.291196 | 132.4 | 245.55 |
| ENSG00000174437 | ATP2A2 | Hs.506759 | 203.5 | 255.4 |
| ENSG00000119866 | BCL11A | Hs.370549 | 363.1 | 387.8 |
| ENSG00000171791 | BCL2 | Hs.150749 | 151.5 | 134.45 |
| ENSG00000029363 | BCLAF1 | Hs.486542 | 659.8 | 1252.25 |
| ENSG00000134107 | BHLHB2 | Hs.171825 | 482.8 | 341.5 |
| ENSG00000181915 | C10orf22 | Hs.99821 | 226.3 | 203.45 |
| ENSG00000139746 | C13orf10 | Hs.131715 | 119.8 | 178.7 |
| ENSG00000163875 | C1orf149 | Hs.17118 | 322.9 | 274.45 |
| ENSG00000117616 | C1orf63 | Hs.259412 | 1202.1 | 1000.85 |
| ENSG00000031003 | C5orf5 | Hs.483473 | 256.3 | 316.85 |
| ENSG00000132424 | C6orf111 | Hs.520287 | 674.8 | 743.75 |
| ENSG00000112308 | C6orf62 | Hs.519930 | 330.6 | 509.45 |
| ENSG00000074410 | CA12 | Hs.210995 | 125.1 | 133.8 |
| ENSG00000135932 | CAB39 | Hs.471593 | 527.9 | 637.3 |
| ENSG00000168564 | CARF | Hs.32922 | 72.4 | 130 |
| ENSG00000142453 | CARM1 | Hs.371416 | 141.6 | 105.7 |
| ENSG00000110619 | CARS | Hs.274873 | 822.3 | 683.8 |
| ENSG00000110848 | CD69 | Hs.208854 | 1631.5 | 1082.1 |
| ENSG00000114331 | CENTB2 | Hs.478746 | 117.5 | 82.45 |
| ENSG00000163320 | CGGBP1 | Hs.444818 | 693.4 | 680 |
| ENSG00000177200 | CHD9 | Hs.530936 | 222.6 | 263 |
| ENSG00000111596 | CNOT2 | Hs.133350 | 431.4 | 399.2 |
| ENSG00000108848 | CROP | Hs.130293 | 2166.4 | 1637.55 |
| ENSG00000198561 | CTNND1 | Hs.166011 | 160.6 | 172.65 |
| ENSG00000149187 | CUGBP1 | Hs.530727 | 332.5 | 326.7 |
| ENSG00000048740 | CUGBP2 | Hs.309288 | 159.6 | 334.5 |
| ENSG00000036257 | CUL3 | Hs.372286 | 554.5 | 473.25 |
| ENSG00000121966 | CXCR4 | Hs.421986 | 532.4 | 1919.4 |
| ENSG00000183283 | DAZAP2 | Hs.369761 | 1267.2 | 976.7 |
| ENSG00000124487 | DDX3X | Hs.380774 | 769.8 | 1229 |
| ENSG00000067048 | DDX3Y | Hs.99120 | 302.6 | 309.6 |
| ENSG00000136986 | DERL1 | Hs.241576 | 223 | 240.65 |
| ENSG00000092964 | DPYSL2 | Hs.173381 | 505.1 | 495.1 |
| ENSG00000143507 | DUSP10 | Hs.497822 | 328.9 | 371.8 |
| ENSG00000139318 | DUSP6 | Hs.298654 | 166.6 | 316.85 |
| ENSG00000120738 | EGR1 | Hs.326035 | 128.6 | 290.15 |
| ENSG00000092847 | EIF2C1 | Hs.22867 | 292.5 | 296.95 |
| ENSG00000123908 | EIF2C2 | Hs.449415 | 150.2 | 115.85 |
| ENSG00000156976 | EIF4A2 | Hs.478553 | 3569 | 6526.55 |
| ENSG00000132507 | EIF5A | Hs.534314 | 1102.8 | 2858.65 |
| ENSG00000109381 | ELF2 | Hs.260903 | 150.8 | 152.4 |
| ENSG00000120616 | EPC1 | Hs.167805 | 148.9 | 88.7 |
| ENSG00000157554 | ERG | Hs.473819 | 369.4 | 298.05 |
| ENSG00000120705 | ETF1 | Hs.483494 | 253.8 | 461.55 |
| ENSG00000139146 | FAM60A | Hs.505154 | 598.1 | 788 |
| ENSG00000138081 | FBXO11 | Hs.553547 | 155.7 | 259.35 |
| ENSG00000143756 | FBXO28 | Hs.64691 | 106.1 | 70.5 |
| ENSG00000196504 | FNBP3 | Hs.298735 | 158.7 | 311.55 |
| ENSG00000170345 | FOS | Hs.25647 | 1254.3 | 1832.65 |
| ENSG00000114861 | FOXP1 | Hs.431498 | 322.7 | 410.3 |
| ENSG00000138757 | G3BP2 | Hs.303676 | 242.8 | 428.55 |
| ENSG00000165219 | GAPVD1 | Hs.495134 | 250 | 262.35 |
| ENSG00000165678 | GHITM | Hs.352656 | 765.3 | 729.1 |
| ENSG00000115419 | GLS | Hs.116448 | 217.6 | 242.2 |
| ENSG00000127955 | GNAI1 | Hs.134587 | 104.9 | 139.15 |
| ENSG00000106070 | GRB10 | Hs.164060 | 550.7 | 574.15 |
| ENSG00000103342 | GSPT1 | Hs.528780 | 356.7 | 369.6 |
| ENSG00000166503 | HDGFRP3 | Hs.513954 | 139.2 | 156 |
| ENSG00000112406 | HECA | Hs.197644 | 352.2 | 377.45 |
| ENSG00000163349 | HIPK1 | Hs.532363 | 347.7 | 488.5 |
| ENSG00000189403 | HMGB1 | Hs.434102 | 500.1 | 602.95 |
| ENSG00000164104 | HMGB2 | Hs.434953 | 855.9 | 2145.45 |
| ENSG00000177733 | HNRPA0 | Hs.96996 | 655 | 723.4 |
| ENSG00000169045 | HNRPH1 | Hs.202166 | 1986.4 | 2132.6 |
| ENSG00000096746 | HNRPH3 | Hs.499891 | 408.7 | 635.5 |
| ENSG00000165119 | HNRPK | Hs.449171 | 761 | 1269.35 |
| ENSG00000120075 | HOXB5 | Hs.149548 | 322 | 238.15 |
| ENSG00000173744 | HRB | Hs.443507 | 202.6 | 405.7 |
| ENSG00000164164 | HSHIN1 | Hs.270851 | 303.1 | 312.65 |
| ENSG00000044574 | HSPA5 | Hs.522394 | 1536.9 | 3296.1 |
| ENSG00000129351 | ILF3 | Hs.465885 | 191 | 302.25 |
| ENSG00000068383 | INPP5A | Hs.523360 | 302.3 | 279.95 |
| ENSG00000148384 | INPP5E | Hs.120998 | 206.1 | 242.45 |
| ENSG00000145703 | IQGAP2 | Hs.291030 | 537.8 | 457.9 |
| ENSG00000121774 | KHDRBS1 | Hs.445893 | 1363 | 1395.2 |
| ENSG00000131149 | KIAA0182 | Hs.461647 | 273.2 | 316.15 |
| ENSG00000145725 | KIAA0433 | Hs.212046 | 164.5 | 186.45 |
| ENSG00000067082 | KLF6 | Hs.285313 | 684.3 | 579.85 |
| ENSG00000114030 | KPNA1 | Hs.161008 | 121.8 | 91.95 |
| ENSG00000168016 | LBA1 | Hs.517761 | 235.1 | 174.9 |
| ENSG00000143815 | LBR | Hs.435166 | 1166 | 1807.35 |
| ENSG00000148943 | LIN7C | Hs.91393 | 87.3 | 121.85 |
| ENSG00000124831 | LRRFIP1 | Hs.471779 | 429.8 | 408.25 |
| ENSG00000121579 | MAK3 | Hs.269528 | 268.8 | 307 |
| ENSG00000166963 | MAP1A | Hs.194301 | 433.9 | 321.4 |
| ENSG00000055208 | MAP3K7IP2 | Hs.269775 | 219.1 | 142.8 |
| ENSG00000145495 | 6-Mar | Hs.432862 | 597.3 | 536.05 |
| ENSG00000175130 | MARCKSL1 | Hs.75061 | 314.8 | 265.6 |
| ENSG00000168906 | MAT2A | Hs.516157 | 1011.4 | 738.6 |
| ENSG00000164024 | METAP1 | Hs.480364 | 351.6 | 422.25 |
| ENSG00000168282 | MGAT2 | Hs.93338 | 130.6 | 123.5 |
| ENSG00000133606 | MKRN1 | Hs.490347 | 392.7 | 350.15 |
| ENSG00000171843 | MLLT3 | Hs.493585 | 405.3 | 334.5 |
| ENSG00000185787 | MORF4L1 | Hs.374503 | 3350.8 | 4457.05 |
| ENSG00000123562 | MORF4L2 | Hs.326387 | 2189.8 | 2626.1 |
| ENSG00000101928 | MOSPD1 | Hs.378505 | 121.1 | 171.3 |
| ENSG00000005810 | MYCBP2 | Hs.151411 | 187 | 643.55 |
| ENSG00000133026 | MYH10 | Hs.16355 | 375.1 | 362.5 |
| ENSG00000136504 | MYST2 | Hs.21907 | 331.7 | 192.25 |
| ENSG00000138386 | NAB1 | Hs.107474 | 187.9 | 203.9 |
| ENSG00000131507 | NDFIP1 | Hs.9788 | 112 | 90.45 |
| ENSG00000137420 | NOL7 | Hs.549161 | 1095.5 | 1222.05 |
| ENSG00000181163 | NPM1 | Hs.519452 | 8905.6 | 11897.7 |
| ENSG00000153234 | NR4A2 | Hs.165258 | 124.6 | 242.85 |
| ENSG00000180530 | NRIP1 | Hs.155017 | 842 | 914.85 |
| ENSG00000035681 | NSMAF | Hs.372000 | 517.1 | 517.75 |
| ENSG00000124789 | NUP153 | Hs.121088 | 216.4 | 291.8 |
| ENSG00000115758 | ODC1 | Hs.467701 | 843.3 | 1634.7 |
| ENSG00000147162 | OGT | Hs.405410 | 165.1 | 183.6 |
| ENSG00000182606 | OIP106 | Hs.535711 | 319.3 | 255.5 |
| ENSG00000091039 | OSBPL8 | Hs.430849 | 212 | 251.65 |
| ENSG00000100836 | PABPN1 | Hs.117176 | 758.2 | 790.85 |
| ENSG00000145730 | PAM | Hs.369430 | 187.3 | 178.45 |
| ENSG00000115421 | PAPOLG | Hs.387471 | 153.2 | 91.25 |
| ENSG00000056661 | PCGF2 | Hs.371617 | 231.6 | 160.8 |
| ENSG00000081154 | PCNP | Hs.549185 | 281.6 | 507.95 |
| ENSG00000059758 | PCTK2 | Hs.506415 | 118.3 | 220.9 |
| ENSG00000114209 | PDCD10 | Hs.478150 | 463.6 | 560.4 |
| ENSG00000150593 | PDCD4 | Hs.232543 | 174.3 | 122.55 |
| ENSG00000143870 | PDIA6 | Hs.212102 | 357.6 | 852.3 |
| ENSG00000197329 | PELI1 | Hs.7886 | 83.3 | 154.5 |
| ENSG00000132326 | PER2 | Hs.58756 | 162.6 | 308.65 |
| ENSG00000043143 | PHF15 | Hs.483419 | 234 | 320.3 |
| ENSG00000118482 | PHF3 | Hs.348921 | 260 | 268.9 |
| ENSG00000073921 | PICALM | Hs.163893 | 357.3 | 315.6 |
| ENSG00000104886 | PLEKHJ1 | Hs.501353 | 436 | 272.7 |
| ENSG00000102024 | PLS3 | Hs.496622 | 74.1 | 110.95 |
| ENSG00000136040 | PLXNC1 | Hs.551522 | 354.6 | 387.1 |
| ENSG00000112941 | POLS | Hs.481542 | 355.5 | 367.35 |
| ENSG00000138032 | PPM1B | Hs.416769 | 79.1 | 82.15 |
| ENSG00000163806 | PPP1CB | Hs.468018 | 65.7 | 135.05 |
| ENSG00000197746 | PSAP | Hs.523004 | 1015.2 | 647.25 |
| ENSG00000125818 | PSMF1 | Hs.471917 | 254.5 | 259.45 |
| ENSG00000117569 | PTBP2 | Hs.269895 | 183 | 160.15 |
| ENSG00000134644 | PUM1 | Hs.281707 | 378.4 | 422.3 |
| ENSG00000185129 | PURA | Hs.443121 | 149.5 | 224.95 |
| ENSG00000112531 | QKI | Hs.510324 | 284.1 | 564.45 |
| ENSG00000103769 | RAB11A | Hs.321541 | 1087.7 | 1020.15 |
| ENSG00000119396 | RAB14 | Hs.371563 | 140.5 | 198.15 |
| ENSG00000175582 | RAB6A | Hs.503222 | 371.4 | 365.75 |
| ENSG00000164754 | RAD21 | Hs.81848 | 281.3 | 414.9 |
| ENSG00000108175 | RAI17 | Hs.193118 | 574.3 | 778.8 |
| ENSG00000132341 | RAN | Hs.10842 | 1434.8 | 2597 |
| ENSG00000065150 | RANBP5 | Hs.513057 | 123.7 | 118.85 |
| ENSG00000010017 | RANBP9 | Hs.306242 | 381.3 | 489.95 |
| ENSG00000127314 | RAP1B | Hs.369920 | 1093.1 | 1537.75 |
| ENSG00000138698 | RAP1GDS1 | Hs.132858 | 169 | 213.85 |
| ENSG00000109756 | RAPGEF2 | Hs.113912 | 394.9 | 551.95 |
| ENSG00000102054 | RBBP7 | Hs.495755 | 942.6 | 1356.35 |
| ENSG00000086589 | RBM22 | Hs.202023 | 339.1 | 441.85 |
| ENSG00000119707 | RBM25 | Hs.531106 | 231.5 | 319.9 |
| ENSG00000168214 | RBPSUH | Hs.479396 | 79.3 | 100.35 |
| ENSG00000089902 | RCOR1 | Hs.510521 | 118.3 | 144.05 |
| ENSG00000176624 | RKHD2 | Hs.465144 | 210.8 | 216 |
| ENSG00000170633 | RNF34 | Hs.292804 | 424.6 | 385.65 |
| ENSG00000043093 | RP42 | Hs.104613 | 236.5 | 204.45 |
| ENSG00000130779 | RSN | Hs.524809 | 76.6 | 58.05 |
| ENSG00000137815 | RTF1 | Hs.511096 | 194.3 | 256.85 |
| ENSG00000159216 | RUNX1 | Hs.149261 | 108.8 | 211.2 |
| ENSG00000163602 | RYBP | Hs.7910 | 398 | 462.8 |
| ENSG00000079332 | SARA1 | Hs.499960 | 233.7 | 442.85 |
| ENSG00000151967 | SCHIP1 | Hs.134665 | 497 | 417.95 |
| ENSG00000156642 | SDFR1 | Hs.187866 | 169.4 | 193.05 |
| ENSG00000168758 | SEMA4C | Hs.516220 | 186.7 | 157.95 |
| ENSG00000179833 | SERTAD2 | Hs.77293 | 498.4 | 556.2 |
| ENSG00000096717 | SIRT1 | Hs.369779 | 133.8 | 124.9 |
| ENSG00000165646 | SLC18A2 | Hs.369009 | 271.3 | 225.6 |
| ENSG00000147454 | SLC25A37 | Hs.122514 | 103 | 86.95 |
| ENSG00000059804 | SLC2A3 | Hs.419240 | 275.9 | 351.5 |
| ENSG00000134294 | SLC38A2 | Hs.221847 | 634.3 | 1026.55 |
| ENSG00000112305 | SMAP1 | Hs.485717 | 205.2 | 129.15 |
| ENSG00000077147 | SMBP | Hs.500674 | 358.3 | 250.05 |
| ENSG00000159140 | SON | Hs.517262 | 408.8 | 467.15 |
| ENSG00000115904 | SOS1 | Hs.278733 | 144 | 169.4 |
| ENSG00000067066 | SP100 | Hs.369056 | 171.3 | 158.85 |
| ENSG00000091640 | SPAG7 | Hs.90436 | 900.7 | 517.9 |
| ENSG00000135250 | SRPK2 | Hs.285197 | 106.2 | 118.7 |
| ENSG00000198648 | STK39 | Hs.276271 | 129.8 | 111.35 |
| ENSG00000117632 | STMN1 | Hs.209983 | 546.6 | 663.4 |
| ENSG00000168952 | STXBP6 | Hs.508958 | 152.6 | 218.55 |
| ENSG00000008282 | SYPL1 | Hs.80919 | 157.3 | 190.55 |
| ENSG00000120948 | TARDBP | Hs.300624 | 735 | 992.75 |
| ENSG00000028839 | TBPL1 | Hs.486507 | 170.1 | 187.95 |
| ENSG00000113649 | TCERG1 | Hs.443465 | 196.5 | 257.25 |
| ENSG00000136205 | TENS1 | Hs.520814 | 443.4 | 221.15 |
| ENSG00000198176 | TFDP1 | Hs.79353 | 156.5 | 362.8 |
| ENSG00000198561 | TMX2 | Hs.279861 | 484.9 | 411 |
| ENSG00000166598 | TRA1 | Hs.192374 | 151 | 339.35 |
| ENSG00000165699 | TSC1 | Hs.370854 | 232.7 | 242.3 |
| ENSG00000119048 | UBE2B | Hs.385986 | 335 | 417.45 |
| ENSG00000160714 | UBE2Q | Hs.516587 | 457.1 | 377.95 |
| ENSG00000124208 | UBE2V1 /// Kua- | Hs.420529 | 504.3 | 320.6 |
| | UEV | | | |
| ENSG00000009335 | UBE3C | Hs.118351 | 67.5 | 175.9 |
| ENSG00000153560 | UBP1 | Hs.118041 | 280.2 | 408.6 |
| ENSG00000135655 | USP15 | Hs.434951 | 197.3 | 160.35 |
| ENSG00000077254 | USP33 | Hs.480597 | 228.7 | 299.75 |
| ENSG00000108828 | VAT1 | Hs.514199 | 649.7 | 585.4 |
| ENSG00000144560 | VGLL4 | Hs.373959 | 299.7 | 175.6 |
| ENSG00000109046 | WSB1 | Hs.446017 | 679.3 | 880.75 |
| ENSG00000083896 | YT521 | Hs.175955 | 318.2 | 505.9 |
| ENSG00000100811 | YY1 | Hs.388927 | 539.7 | 740.2 |
| ENSG00000107372 | ZA20D2 | Hs.406096 | 967.2 | 1262.9 |
| ENSG00000086666 | ZA20D3 | Hs.306329 | 735.1 | 517.75 |
| ENSG00000058673 | ZC3H11A | Hs.532399 | 665.7 | 989.4 |
| ENSG00000153786 | ZDHHC7 | Hs.461610 | 713.7 | 620.1 |
| ENSG00000152518 | ZFP36L2 | Hs.503093 | 4553.7 | 5205.6 |
| ENSG00000170949 | ZNF160 | Hs.467236 | 1056.4 | 1016.85 |
| ENSG00000179456 | ZNF238 | Hs.69997 | 235.7 | 241.8 |

| | | | | | | |
|---|---|---|---|---|---|---|
| Ensemble Gene | | | PBSC | PBSC | | BM |
| ID | Gene Symbol | Unigene | RFU | Flags | BM RFU | Flags |

193

| | | | | | | |
|---|---|---|---|---|---|---|
| ENSG00000161203 | AP2M1 | Hs.518460 | 687.4 | PP | 387.65 | PA |
| ENSG00000108561 | C1QBP | Hs.553487 | 302.1 | PP | 421.75 | PP |
| ENSG00000104327 | CALB1 | Hs.65425 | 79.6 | PP | 42.05 | PA |
| ENSG00000102974 | CTCF | Hs.368367 | 506.1 | PP | 535.55 | PP |
| ENSG00000075711 | DLG1 | Hs.292549 | 327.5 | PP | 426.8 | PP |
| ENSG00000086061 | DNAJA1 | Hs.445203 | 1073.8 | PP | 1437.15 | PP |
| ENSG00000120699 | EXOSC8 | Hs.294041 | 196.9 | PP | 283.25 | PP |
| ENSG00000179115 | FARSLA | Hs.23111 | 250.6 | PP | 414.1 | PP |
| ENSG00000138081 | FBXO11 | Hs.553547 | 155.7 | PP | 259.35 | PP |
| ENSG00000153487 | ING1 | Hs.46700 | 247.1 | PP | 176.1 | PA |
| ENSG00000068383 | INPP5A | Hs.523360 | 302.3 | PP | 279.95 | PP |
| ENSG00000135387 | M11S1 | Hs.471818 | 464.9 | PP | 451.45 | PP |
| ENSG00000125952 | MAX | Hs.285354 | 1178.7 | PP | 1480.1 | PP |
| ENSG00000082641 | NFE2L1 | Hs.514284 | 735.5 | PP | 726.35 | PP |
| ENSG00000116044 | NFE2L2 | Hs.155396 | 181.2 | PP | 280.9 | PP |
| ENSG00000180530 | NRIP1 | Hs.155017 | 842 | PP | 914.85 | PP |
| ENSG00000169564 | PCBP1 | Hs.2853 | 1587.4 | PP | 1289.35 | PP |
| ENSG00000056661 | PCGF2 | Hs.371617 | 231.6 | PP | 160.8 | PP |
| ENSG00000162734 | PEA15 | Hs.517216 | 180.3 | PP | 156.05 | PP |
| ENSG00000136040 | PLXNC1 | Hs.551522 | 354.6 | PP | 387.1 | PP |
| ENSG00000104762 | PPP2R2A | Hs.146339 | 331.7 | PP | 299.6 | PP |
| ENSG00000078304 | PPP2R5C | Hs.368264 | 174.9 | PP | 165.25 | PP |
| ENSG00000056687 | RGL2 | Hs.509622 | 340.5 | PP | 232.1 | PA |
| ENSG00000043093 | RP42 | Hs.104613 | 236.5 | PP | 204.45 | PP |
| ENSG00000159140 | SON | Hs.517262 | 408.8 | PP | 467.15 | PP |
| ENSG00000166301 | STK19 | Hs.485102 | 527.6 | PP | 424 | PA |
| ENSG00000117632 | STMN1 | Hs.209983 | 546.6 | PP | 663.4 | PP |
| ENSG00000124222 | STX16 | Hs.307913 | 474.2 | PP | 228.9 | PP |
| ENSG00000120948 | TARDBP | Hs.300624 | 735 | PP | 992.75 | PP |
| ENSG00000166326 | TRIM44 | Hs.192103 | 340.6 | PP | 375.85 | PP |
| ENSG00000119801 | YPEL5 | Hs.515890 | 1377.9 | PP | 1469.25 | PP |

24

| | | | | | | |
|---|---|---|---|---|---|---|
| ENSG00000148700 | ADD3 | Hs.501012 | 258.6 | PP | 299.05 | PP |
| ENSG00000168374 | ARF4 | Hs.148330 | 129 | PP | 157.5 | PP |
| ENSG00000143153 | ATP1B1 | Hs.291196 | 132.4 | PP | 245.55 | PP |
| ENSG00000174437 | ATP2A2 | Hs.506759 | 203.5 | PP | 255.4 | PP |
| ENSG00000119866 | BCL11A | Hs.370549 | 363.1 | PP | 387.8 | PP |
| ENSG00000095564 | BTAF1 | Hs.500526 | 220.4 | PP | 333.3 | PP |
| ENSG00000082153 | BZW1 | Hs.355983 | 536.1 | PP | 794.1 | PP |
| ENSG00000181915 | C10orf22 | Hs.99821 | 226.3 | PP | 203.45 | PP |
| ENSG00000179218 | CALR | Hs.515162 | 398.5 | PP | 1371.55 | PP |
| ENSG00000118816 | CCNI | Hs.518827 | 913.5 | PP | 1344.25 | PP |
| ENSG00000163468 | CCT3 | Hs.491494 | 1044.4 | PP | 1212.8 | PP |
| ENSG00000135535 | CD164 | Hs.520313 | 2274.9 | PP | 2265.25 | PP |
| ENSG00000111276 | CDKN1B | Hs.238990 | 333.5 | PP | 374.65 | PP |
| ENSG00000143126 | CELSR2 | Hs.57652 | 215.4 | PP | 148.65 | PP |
| ENSG00000109572 | CLCN3 | Hs.481186 | 244.9 | PP | 248.15 | PP |
| ENSG00000118260 | CREB1 | Hs.516646 | 122.9 | PP | 230.35 | PP |
| ENSG00000111269 | CREBL2 | Hs.13313 | 353.2 | PP | 216.25 | PP |
| ENSG00000048740 | CUGBP2 | Hs.309288 | 159.6 | PP | 334.5 | PP |
| ENSG00000165659 | DACH1 | Hs.129452 | 95.1 | PP | 144.8 | PP |
| ENSG00000132153 | DHX30 | Hs.517948 | 209.5 | PP | 193.5 | PP |
| ENSG00000082458 | DLG3 | Hs.522680 | 136.2 | PP | 89.45 | PP |
| ENSG00000120738 | EGR1 | Hs.326035 | 128.6 | PP | 290.15 | PP |
| ENSG00000139146 | FAM60A | Hs.505154 | 598.1 | PP | 788 | PP |
| ENSG00000105216 | FAM61A | Hs.407368 | 242.1 | PP | 291.3 | PP |
| ENSG00000118564 | FBXL5 | Hs.479208 | 410.6 | PP | 319.15 | PP |
| ENSG00000138081 | FBXO11 | Hs.553547 | 155.7 | PP | 259.35 | PA |
| ENSG00000068078 | FGFR3 | Hs.1420 | 185.4 | PP | 135.5 | PA |
| ENSG00000175606 | FLJ20533 | Hs.106650 | 420.6 | PP | 454.55 | PP |
| ENSG00000034713 | GABARAPL2 | Hs.461379 | 952 | PP | 625.75 | PP |
| ENSG00000188486 | H2AFX | Hs.477879 | 97.1 | PP | 383.1 | PA |
| ENSG00000091527 | H41 | Hs.518265 | 735.1 | PP | 1129.65 | PP |
| ENSG00000010818 | HIVEP2 | Hs.510172 | 149.6 | PP | 157.9 | PP |
| ENSG00000106004 | HOXA5 | Hs.37034 | 507.2 | PP | 499.3 | PP |
| ENSG00000074356 | HSA277841 | Hs.120963 | 222.9 | PP | 150.05 | PP |
| ENSG00000068745 | IHPK2 | Hs.476221 | 648.7 | PP | 414.15 | PP |
| ENSG00000105135 | ILVBL | Hs.78880 | 243.6 | PP | 297 | PP |
| ENSG00000186480 | INSIG1 | Hs.520819 | 196.9 | PP | 304.7 | PP |
| ENSG00000140575 | IQGAP1 | Hs.430551 | 113 | PP | 199.1 | PP |
| ENSG00000136240 | KDELR2 | Hs.520210 | 123.7 | PP | 161.05 | PM |
| ENSG00000088247 | KHSRP | Hs.91142 | 163.3 | PP | 288.9 | PP |
| ENSG00000102753 | KPNA3 | Hs.527919 | 250.8 | PP | 391.35 | PP |
| ENSG00000102897 | LOC57149 | Hs.185489 | 370.6 | PP | 303.95 | PP |
| ENSG00000077454 | LRCH4 | Hs.125742 | 1133.8 | PP | 712.75 | PP |
| ENSG00000011009 | LYPLA2 /// | Hs.534075 | 585.5 | PP | 486 | PP |
| | LYPLA2P1 /// | | | | | |
| | LOC388499 | | | | | |
| ENSG00000135387 | M11S1 | Hs.471818 | 464.9 | PP | 451.45 | PP |
| ENSG00000143033 | M96 | Hs.31016 | 147.6 | PP | 141.55 | PP |
| ENSG00000161021 | MAML1 | Hs.484349 | 331.3 | PP | 280.35 | PP |
| ENSG00000166963 | MAP1A | Hs.194301 | 433.9 | PP | 321.4 | PM |
| ENSG00000100030 | MAPK1 | Hs.431850 | 274.3 | PP | 145 | PP |
| ENSG00000175130 | MARCKSL1 | Hs.75061 | 314.8 | PP | 265.6 | PP |
| ENSG00000166986 | MARS | Hs.355867 | 460.7 | PP | 614.95 | PP |
| ENSG00000015479 | MATR3 | Hs.268939 | 252.4 | PP | 382.35 | PP |
| ENSG00000133895 | MEN1 | Hs.423348 | 113.9 | PP | 175.85 | PP |
| ENSG00000079277 | MKNK1 | Hs.371594 | 233.1 | PP | 239.5 | PP |
| ENSG00000143033 | MTF2 | Hs.31016 | 559 | PP | 738.65 | PP |
| ENSG00000118513 | MYB | Hs.531941 | 1984.2 | PP | 2558.15 | PP |
| ENSG00000005810 | MYCBP2 | Hs.151411 | 187 | PP | 643.55 | PP |
| ENSG00000133026 | MYH10 | Hs.16355 | 375.1 | PP | 362.5 | PP |
| ENSG00000115053 | NCL | Hs.79110 | 1203.2 | PP | 2603.05 | PP |
| ENSG00000166579 | NDEL1 | Hs.372123 | 218.3 | PP | 187.2 | PP |
| ENSG00000173848 | NET1 | Hs.25155 | 264.1 | PP | 310.45 | PP |
| ENSG00000116044 | NFE2L2 | Hs.155396 | 181.2 | PP | 280.9 | PP |
| ENSG00000158793 | NIT1 | Hs.146406 | 409.1 | PP | 328.05 | PP |
| ENSG00000149308 | NPAT | Hs.171061 | 171.1 | PP | 140.65 | PP |
| ENSG00000147162 | OGT | Hs.405410 | 165.1 | PP | 183.6 | PP |
| ENSG00000141447 | OSBPL1A | Hs.370725 | 204.6 | PP | 211.75 | PP |
| ENSG00000091039 | OSBPL8 | Hs.430849 | 212 | PP | 251.65 | PP |
| ENSG00000102225 | PCTK1 | Hs.496068 | 135 | PP | 88.45 | PA |
| ENSG00000059758 | PCTK2 | Hs.506415 | 118.3 | PP | 220.9 | PP |
| ENSG00000137193 | PIM1 | Hs.81170 | 1021.7 | PP | 801.85 | PP |
| ENSG00000102096 | PIM2 | Hs.496096 | 251.9 | PP | 288.3 | PM |
| ENSG00000152952 | PLOD2 | Hs.477866 | 92.1 | PP | 128.6 | PP |
| ENSG00000100034 | PPM1F | Hs.112728 | 179.2 | PP | 108.2 | PA |
| ENSG00000197746 | PSAP | Hs.523004 | 1015.2 | PP | 647.25 | PP |
| ENSG00000106588 | PSMA2 | Hs.333786 | 204.2 | PP | 240.35 | PP |
| ENSG00000117569 | PTBP2 | Hs.269895 | 183 | PP | 160.15 | PP |
| ENSG00000171522 | PTGER4 | Hs.199248 | 531.8 | PP | 514.8 | PP |
| ENSG00000185129 | PURA | Hs.443121 | 149.5 | PP | 224.95 | PP |
| ENSG00000112531 | QKI | Hs.510324 | 284.1 | PP | 564.45 | PP |
| ENSG00000111540 | RAB5B | Hs.77690 | 234.1 | PP | 216.45 | PP |
| ENSG00000116473 | RAP1A | Hs.190334 | 217.2 | PP | 367.75 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000145715 | RASA1 | Hs.553501 | 138.8 | PP | 103.6 | PP |
| ENSG00000056687 | RGL2 | Hs.509622 | 340.5 | PP | 232.1 | PA |
| ENSG00000123091 | RNF11 | Hs.309641 | 125.3 | PP | 161.5 | PP |
| ENSG00000134758 | RNF138 | Hs.302408 | 186.9 | PP | 511.9 | PP |
| ENSG00000043093 | RP42 | Hs.104613 | 236.5 | PP | 204.45 | PP |
| ENSG00000136715 | SAP130 | Hs.32995 | 229 | PP | 246.4 | PP |
| ENSG00000140497 | SCAMP2 | Hs.458917 | 411.8 | PP | 495 | PP |
| ENSG00000140612 | SEC11L1 | Hs.9534 | 1920.5 | PP | 2226.35 | PP |
| ENSG00000092421 | SEMA6A | Hs.156967 | 140 | PP | 182.2 | PP |
| ENSG00000080546 | SESN1 | Hs.59554 | 235 | PP | 276.4 | PP |
| ENSG00000119335 | SET | Hs.436687 | 2967 | PP | 4307.1 | PP |
| ENSG00000182199 | SHMT2 | Hs.75069 | 828.3 | PP | 865.1 | PP |
| ENSG00000173473 | SMARCC1 | Hs.476179 | 487 | PP | 483.35 | PP |
| ENSG00000184602 | SNN | Hs.459952 | 234.4 | PP | 239.3 | PP |
| ENSG00000100596 | SPTLC2 | Hs.435661 | 433.7 | PP | 462.25 | PP |
| ENSG00000120948 | TARDBP | Hs.300624 | 735 | PP | 992.75 | PP |
| ENSG00000006638 | TBXA2R | Hs.442530 | 258.9 | PP | 217.8 | PP |
| ENSG00000183864 | TOB2 | Hs.474978 | 165.3 | PP | 196.4 | PP |
| ENSG00000173726 | TOMM20 | Hs.533192 | 656.5 | PP | 888.45 | PP |
| ENSG00000198900 | TOP1 | Hs.472737 | 315.5 | PP | 468.35 | PP |
| ENSG00000077097 | TOP2B | Hs.475733 | 592.4 | PP | 806 | PP |
| ENSG00000111669 | TPI1 | Hs.524219 | 1154.9 | PP | 1213.85 | PP |
| ENSG00000140416 | TPM1 | Hs.133892 | 394.4 | PP | 661.4 | PP |
| ENSG00000157514 | TSC22D3 | Hs.522074 | 1752.5 | PP | 2189.45 | PP |
| ENSG00000131508 | UBE2D2 | Hs.108332 | 400.1 | PP | 512.05 | PP |
| ENSG00000114062 | UBE3A | Hs.22543 | 456.9 | PP | 470.75 | PP |
| ENSG00000118900 | UBN1 | Hs.440219 | 138.4 | PP | 302.15 | PP |
| ENSG00000141968 | VAV1 | Hs.116237 | 535.2 | PP | 272.75 | PA |
| ENSG00000100897 | WDR23 | Hs.525251 | 455.1 | PP | 382.25 | PP |
| ENSG00000082898 | XPO1 | Hs.370770 | 994.7 | PP | 1393.85 | PP |
| ENSG00000128245 | YWHAH | Hs.226755 | 195 | PP | 195.4 | PP |
| ENSG00000147130 | ZNF261 | Hs.522684 | 475.8 | PP | 232.55 | PP |

181b

| | | | | | | |
|---|---|---|---|---|---|---|
| ENSG00000168374 | ARF4 | Hs.148330 | 129 | PP | 157.5 | PP |
| ENSG00000143153 | ATP1B1 | Hs.291196 | 132.4 | PP | 245.55 | PP |
| ENSG00000174437 | ATP2A2 | Hs.506759 | 203.5 | PP | 255.4 | PP |
| ENSG00000119866 | BCL11A | Hs.370549 | 363.1 | PP | 387.8 | PP |
| ENSG00000134107 | BHLHB2 | Hs.171825 | 482.8 | PP | 341.5 | PP |
| ENSG00000168564 | CARF | Hs.32922 | 72.4 | PP | 130 | PP |
| ENSG00000113328 | CCNG1 | Hs.79101 | 1025 | PP | 737.8 | PP |
| ENSG00000141367 | CLTC | Hs.491351 | 1169 | PP | 1399.6 | PP |
| ENSG00000111596 | CNOT2 | Hs.133350 | 431.4 | PP | 399.2 | PP |
| ENSG00000108848 | CROP | Hs.130293 | 2166.4 | PP | 1637.55 | PP |
| ENSG00000149187 | CUGBP1 | Hs.530727 | 383 | PP | 596.3 | PP |
| ENSG00000048740 | CUGBP2 | Hs.309288 | 159.6 | PP | 334.5 | PP |
| ENSG00000172115 | CYCS | Hs.437060 | 791.3 | PP | 2063.1 | PP |
| ENSG00000168209 | DDIT4 | Hs.523012 | 736.8 | PP | 535.85 | PP |
| ENSG00000109606 | DHX15 | Hs.5683 | 1219.8 | PP | 1620.75 | PP |
| ENSG00000120738 | EGR1 | Hs.326035 | 128.6 | PP | 290.15 | PP |
| ENSG00000132507 | EIF5A | Hs.534314 | 1102.8 | PP | 2858.65 | PP |
| ENSG00000120616 | EPC1 | Hs.167805 | 148.9 | PP | 88.7 | PA |
| ENSG00000120705 | ETF1 | Hs.483494 | 253.8 | PP | 461.55 | PP |
| ENSG00000139146 | FAM60A | Hs.505154 | 598.1 | PP | 788 | PP |
| ENSG00000138081 | FBXO11 | Hs.553547 | 155.7 | PP | 259.35 | PP |
| ENSG00000106070 | GRB10 | Hs.164060 | 550.7 | PP | 574.15 | PP |
| ENSG00000103342 | GSPT1 | Hs.528780 | 356.7 | PP | 369.6 | PP |
| ENSG00000170144 | HNRPA3 | Hs.516539 | 375.5 | PP | 558.6 | PP |
| ENSG00000126945 | HNRPH2 | Hs.278857 | 189.2 | PP | 158.45 | PP |
| ENSG00000044574 | HSPA5 | Hs.522394 | 1536.9 | PP | 3296.1 | PA |
| ENSG00000142856 | ITGB3BP | Hs.166539 | 234.8 | PP | 372 | PP |
| ENSG00000078596 | ITM2A | Hs.17109 | 325.6 | PP | 367.7 | PP |
| ENSG00000121774 | KHDRBS1 | Hs.445893 | 1363 | PP | 1395.2 | PP |
| ENSG00000108424 | KPNB1 | Hs.532793 | 415.1 | PP | 709.85 | PP |
| ENSG00000168906 | MAT2A | Hs.516157 | 1011.4 | PP | 738.6 | PP |
| ENSG00000123562 | MORF4L2 | Hs.326387 | 2189.8 | PP | 2626.1 | PP |
| ENSG00000170873 | MTSS1 | Hs.336994 | 152.7 | PP | 194.6 | PP |
| ENSG00000133026 | MYH10 | Hs.16355 | 375.1 | PP | 362.5 | PP |
| ENSG00000136504 | MYST2 | Hs.21907 | 331.7 | PP | 192.25 | PP |
| ENSG00000164190 | NIPBL | Hs.481927 | 165.1 | PP | 145.15 | PP |
| ENSG00000124789 | NUP153 | Hs.121088 | 216.4 | PP | 291.8 | PP |
| ENSG00000046651 | OFD1 | Hs.6483 | 1022.7 | PP | 692.15 | PP |
| ENSG00000091039 | OSBPL8 | Hs.430849 | 212 | PP | 251.65 | PP |
| ENSG00000172239 | PAIP1 | Hs.482038 | 127.8 | PP | 157 | PP |
| ENSG00000157654 | PALM2- | Hs.259461 | 181.4 | PP | 163.75 | PP |
| | AKAP2 | | | | | |
| ENSG00000145730 | PAM | Hs.369430 | 187.3 | PP | 178.45 | PP |
| ENSG00000115421 | PAPOLG | Hs.387471 | 153.2 | PP | 91.25 | PP |
| ENSG00000059758 | PCTK2 | Hs.506415 | 118.3 | PP | 220.9 | PP |
| ENSG00000065485 | PDIA5 | Hs.477352 | 352.8 | PP | 418.95 | PP |
| ENSG00000138814 | PPP3CA | Hs.435512 | 122.5 | PP | 205 | PP |
| ENSG00000125818 | PSMF1 | Hs.471917 | 254.5 | PP | 259.45 | PP |
| ENSG00000121390 | PSPC1 | Hs.213198 | 142.5 | PP | 179.5 | PP |
| ENSG00000138430 | PTD004 | Hs.157351 | 848.2 | PP | 1052.6 | PP |
| ENSG00000134644 | PUM1 | Hs.281707 | 378.4 | PP | 422.3 | PP |
| ENSG00000185129 | PURA | Hs.443121 | 149.5 | PP | 224.95 | PP |
| ENSG00000010017 | RANBP9 | Hs.306242 | 381.3 | PP | 489.95 | PP |
| ENSG00000163602 | RYBP | Hs.7910 | 398 | PP | 462.8 | PP |
| ENSG00000168758 | SEMA4C | Hs.516220 | 186.7 | PP | 157.95 | PP |
| ENSG00000116560 | SFPQ | Hs.355934 | 306.9 | PP | 689 | PP |
| ENSG00000096717 | SIRT1 | Hs.369779 | 133.8 | PP | 124.9 | PP |
| ENSG00000095480 | SPFH1 | Hs.150087 | 646.4 | PP | 689.15 | PP |
| ENSG00000124783 | SSR1 | Hs.114033 | 600.7 | PP | 686.65 | PP |
| ENSG00000168610 | STAT3 | Hs.463059 | 401.7 | PP | 390.05 | PP |
| ENSG00000102572 | STK24 | Hs.508514 | 958.4 | PP | 1363.75 | PP |
| ENSG00000168952 | STXBP6 | Hs.508958 | 152.6 | PP | 218.55 | PP |
| ENSG00000028839 | TBPL1 | Hs.486507 | 170.1 | PP | 187.95 | PP |
| ENSG00000136205 | TENS1 | Hs.520814 | 443.4 | PP | 221.15 | PP |
| ENSG00000077097 | TOP2B | Hs.475733 | 592.4 | PP | 806 | PP |
| ENSG00000166598 | TRA1 | Hs.192374 | 151 | PP | 339.35 | PP |
| ENSG00000165699 | TSC1 | Hs.370854 | 232.7 | PP | 242.3 | PP |
| ENSG00000134215 | VAV3 | Hs.267659 | 615.4 | PP | 341.3 | PP |
| ENSG00000132716 | WDR42A | Hs.492236 | 195.9 | PP | 233.1 | PP |
| ENSG00000170949 | ZNF160 | Hs.467236 | 1056.4 | PP | 1016.85 | PP |
| ENSG00000179456 | ZNF238 | Hs.69997 | 235.7 | PP | 241.8 | PP |

let-7c

| | | | | | | |
|---|---|---|---|---|---|---|
| ENSG00000114770 | ABCC5 | Hs.368563 | 373.4 | PP | 322.1 | PM |
| ENSG00000184009 | ACTG1 | Hs.514581 | 11731 | PP | 14874 | PP |
| ENSG00000151320 | AKAP6 | Hs.509083 | 137.8 | PP | 117.05 | PP |
| ENSG00000123505 | AMD1 | Hs.159118 | 648.3 | PP | 1011.85 | PP |
| ENSG00000062725 | APPBP2 | Hs.84084 | 106.4 | PP | 116.15 | PA |
| ENSG00000175220 | ARHGAP1 | Hs.138860 | 594 | PP | 502.3 | PP |
| ENSG00000117713 | ARID1A | Hs.468972 | 477.6 | PP | 444.8 | PP |
| ENSG00000116539 | ASH1L | Hs.491060 | 244.1 | PP | 214.3 | PA |
| ENSG00000171456 | ASXL1 | Hs.374043 | 275.6 | PP | 307.25 | PP |
| ENSG00000082153 | BZW1 | Hs.355983 | 536.1 | PP | 794.1 | PP |
| ENSG00000136261 | BZW2 | Hs.487635 | 1167.4 | PP | 1664.45 | PP |
| ENSG00000104327 | CALB1 | Hs.65425 | 79.6 | PP | 42.05 | PA |
| ENSG00000131236 | CAP1 | Hs.370581 | 765.9 | PP | 691 | PP |
| ENSG00000112576 | CCND3 | Hs.534307 | 539.8 | PP | 402.15 | PP |
| ENSG00000111642 | CHD4 | Hs.162233 | 299.5 | PP | 431.1 | PP |
| ENSG00000100528 | CNIH | Hs.294603 | 252.6 | PP | 391.7 | PP |
| ENSG00000108848 | CROP | Hs.130293 | 2166.4 | PP | 1637.55 | PP |
| ENSG00000009307 | CSDE1 | Hs.69855 | 1494.1 | PP | 1637.9 | PP |
| ENSG00000101266 | CSNK2A1 | Hs.446484 | 170.4 | PP | 261.4 | PP |
| ENSG00000048740 | CUGBP2 | Hs.309288 | 159.6 | PP | 334.5 | PP |
| ENSG00000119689 | DLST /// | Hs.480230 | 187.3 | PP | 148.8 | PM |
| | DLSTP | | | | | |
| ENSG00000161202 | DVL3 | Hs.388116 | 134.8 | PP | 206.95 | PP |
| ENSG00000092847 | EIF2C1 | Hs.22867 | 292.5 | PP | 296.95 | PP |
| ENSG00000104131 | EIF3S1 | Hs.404056 | 340.4 | PP | 365.05 | PP |
| ENSG00000110321 | EIF4G2 | Hs.183684 | 2137 | PP | 2701.9 | PP |
| ENSG00000109381 | ELF2 | Hs.260903 | 150.8 | PP | 152.4 | PA |
| ENSG00000100442 | FKBP3 | Hs.509226 | 386.9 | PP | 438.15 | PP |
| ENSG00000141429 | GALNT1 | Hs.514806 | 220 | PP | 273.8 | PP |
| ENSG00000108588 | GK001 | Hs.202011 | 160.3 | PP | 198.45 | PP |
| ENSG00000174021 | GNG5 | Hs.513557 | 683.5 | PP | 869.7 | PP |
| ENSG00000103342 | GSPT1 | Hs.528780 | 356.7 | PP | 369.6 | PP |
| ENSG00000152795 | HNRPDL | Hs.527105 | 1931.8 | PP | 1802.05 | PP |
| ENSG00000078399 | HOXA9 | Hs.127428 | 739.2 | PP | 674 | PP |
| ENSG00000109083 | IFT20 | Hs.4187 | 322.8 | PP | 241.4 | PP |
| ENSG00000105135 | ILVBL | Hs.78880 | 243.6 | PP | 297 | PP |
| ENSG00000140575 | IQGAP1 | Hs.430551 | 113 | PP | 199.1 | PP |
| ENSG00000171988 | JMJD1C | Hs.413416 | 107 | PP | 198.85 | PP |
| ENSG00000100097 | LGALS1 | Hs.445351 | 454.5 | PP | 244.1 | PP |
| ENSG00000111252 | LNK | Hs.506784 | 230.1 | PP | 198.8 | PP |
| ENSG00000145012 | LPP | Hs.444362 | 205.6 | PP | 238.9 | PP |
| ENSG00000054219 | LY75 | Hs.153563 | 126.4 | PP | 175.65 | PP |
| ENSG00000166963 | MAP1A | Hs.194301 | 433.9 | PP | 321.4 | PM |
| ENSG00000075413 | MARK3 | Hs.35828 | 129.9 | PP | 75.05 | PP |
| ENSG00000147065 | MSN | Hs.87752 | 793.3 | PP | 791.75 | PP |
| ENSG00000100330 | MTMR3 | Hs.474536 | 204.7 | PP | 287 | PP |
| ENSG00000138386 | NAB1 | Hs.107474 | 187.9 | PP | 203.9 | PP |
| ENSG00000106459 | NRF1 | Hs.298069 | 157.5 | PP | 126.8 | PA |
| ENSG00000184588 | PDE4B | Hs.198072 | 234.1 | PP | 349.5 | PP |
| ENSG00000136040 | PLXNC1 | Hs.551522 | 354.6 | PP | 387.1 | PP |
| ENSG00000119396 | RAB14 | Hs.371563 | 140.5 | PP | 198.15 | PP |
| ENSG00000111540 | RAB5B | Hs.77690 | 234.1 | PP | 216.45 | PP |
| ENSG00000056687 | RGL2 | Hs.509622 | 340.5 | PP | 232.1 | PA |
| ENSG00000101782 | RIOK3 | Hs.445511 | 276.1 | PP | 311.7 | PP |
| ENSG00000137075 | RNF38 | Hs.333503 | 322.6 | PP | 227.7 | PP |
| ENSG00000151967 | SCHIP1 | Hs.134665 | 497 | PP | 417.95 | PP |
| ENSG00000155878 | SEC22L1 | Hs.534212 | 662.9 | PP | 588.95 | PP |
| ENSG00000168758 | SEMA4C | Hs.516220 | 186.7 | PP | 157.95 | PP |
| ENSG00000120742 | SERP1 | Hs.518326 | 285.7 | PP | 343.4 | PP |
| ENSG00000153914 | SFRS12 | Hs.519347 | 258 | PP | 229.6 | PP |
| ENSG00000102038 | SMARCA1 | Hs.152292 | 101.1 | PP | 72.95 | PA |
| ENSG00000124783 | SSR1 | Hs.114033 | 600.7 | PP | 686.65 | PP |
| ENSG00000168610 | STAT3 | Hs.463059 | 401.7 | PP | 390.05 | PP |
| ENSG00000119048 | UBE2B | Hs.385986 | 335 | PP | 417.45 | PP |
| ENSG00000109332 | UBE2D3 | Hs.472031 | 471.2 | PP | 635.4 | PP |
| ENSG00000135655 | USP15 | Hs.434951 | 197.3 | PP | 160.35 | PP |
| ENSG00000134215 | VAV3 | Hs.267659 | 615.4 | PP | 341.3 | PP |
| ENSG00000073905 | VDAC1 | Hs.202085 | 175.6 | PP | 289.2 | PP |
| ENSG00000122958 | VPS26 | Hs.499925 | 508.7 | PP | 610 | PP |
| ENSG00000166913 | YWHAB | Hs.279920 | 222.4 | PP | 288.75 | PP |
| ENSG00000107372 | ZA20D2 | Hs.406096 | 967.2 | PP | 1262.9 | PP |
| ENSG00000015171 | ZMYND11 | Hs.292265 | 403.3 | PP | 784.1 | PP |
| ENSG00000147130 | ZNF261 | Hs.522684 | 475.8 | PP | 232.55 | PP |

192-2_3

| | | | | | | |
|---|---|---|---|---|---|---|
| ENSG00000138071 | ACTR2 | Hs.393201 | 343.5 | PP | 1110.1 | PP |
| ENSG00000170017 | ALCAM | Hs.150693 | 528.9 | PP | 695.45 | PP |
| ENSG00000123268 | ATF1 | Hs.435267 | 95.1 | PP | 109.2 | PP |
| ENSG00000119866 | BCL11A | Hs.370549 | 363.1 | PP | 387.8 | PP |
| ENSG00000134153 | C15orf24 | Hs.160565 | 612.5 | PP | 706.2 | PP |
| ENSG00000082258 | CCNT2 | Hs.292754 | 114.4 | PP | 102.75 | PP |
| ENSG00000111269 | CREBL2 | Hs.13313 | 353.2 | PP | 216.25 | PP |
| ENSG00000137992 | DBT | Hs.270570 | 2078.5 | PP | 2456.8 | PP |
| ENSG00000124487 | DDX3X | Hs.380774 | 769.8 | PP | 1229 | PP |
| ENSG00000157540 | DYRK1A | Hs.368240 | 367.8 | PP | 751.2 | PP |
| ENSG00000124882 | EREG | Hs.115263 | 351.8 | PP | 535.15 | PP |
| ENSG00000153807 | HOXA10 | Hs.110637 | 161.3 | PP | 200.7 | PA |
| ENSG00000150093 | ITGB1 | Hs.429052 | 865.6 | PP | 593.85 | PP |
| ENSG00000119321 | KIAA0674 | Hs.522351 | 310 | PP | 302.4 | PP |
| ENSG00000127603 | MACF1 | Hs.472475 | 222.8 | PP | 248.35 | PP |
| ENSG00000175130 | MARCKSL1 | Hs.75061 | 314.8 | PP | 265.6 | PP |
| ENSG00000147065 | MSN | Hs.87752 | 793.3 | PP | 791.75 | PP |
| ENSG00000108389 | MTMR4 | Hs.514373 | 330.7 | PP | 407.2 | PP |
| ENSG00000138386 | NAB1 | Hs.107474 | 187.9 | PP | 203.9 | PP |
| ENSG00000137420 | NOL7 | Hs.549161 | 1095.5 | PP | 1222.05 | PP |
| ENSG00000180530 | NRIP1 | Hs.155017 | 842 | PP | 914.85 | PP |
| ENSG00000090621 | PABPC4 | Hs.169900 | 376 | PP | 622.5 | PP |
| ENSG00000114054 | PCCB | Hs.63788 | 270.6 | PP | 392.75 | PP |
| ENSG00000107758 | PPP3CB | Hs.500067 | 318.9 | PP | 241.25 | PP |
| ENSG00000117569 | PTBP2 | Hs.269895 | 183 | PP | 160.15 | PP |
| ENSG00000112531 | QKI | Hs.510324 | 284.1 | PP | 564.45 | PA |
| ENSG00000139687 | RB1 | Hs.408528 | 152.7 | PP | 153.35 | PP |
| ENSG00000137075 | RNF38 | Hs.333503 | 322.6 | PP | 227.7 | PP |
| ENSG00000159216 | RUNX1 | Hs.149261 | 108.8 | PP | 211.2 | PP |
| ENSG00000179833 | SERTAD2 | Hs.77293 | 498.4 | PP | 556.2 | PP |
| ENSG00000124193 | SFRS6 | Hs.6891 | 2001.1 | PP | 2200.75 | PP |
| ENSG00000147454 | SLC25A37 | Hs.122514 | 103 | PP | 86.95 | PA |
| ENSG00000124222 | STX16 | Hs.307913 | 474.2 | PP | 228.9 | PP |
| ENSG00000132718 | SYT11 | Hs.32984 | 106.3 | PP | 102.95 | PP |
| ENSG00000109332 | UBE2D3 | Hs.472031 | 471.2 | PP | 635.4 | PP |
| ENSG00000162607 | USP1 | Hs.35086 | 275.3 | PP | 614.05 | PP |

24-2

see 24

128a

| | | | | | | |
|---|---|---|---|---|---|---|
| ENSG00000138031 | ADCY3 | Hs.467898 | 208.6 | PP | 178.4 | PP |
| ENSG00000004455 | AK2 | Hs.470907 | 303.6 | PP | 498.6 | PP |
| ENSG00000123505 | AMD1 | Hs.159118 | 648.3 | PP | 1011.85 | PP |
| ENSG00000120868 | APAF1 | Hs.552567 | 65.9 | PP | 94.15 | PP |
| ENSG00000062725 | APPBP2 | Hs.84084 | 106.4 | PP | 116.15 | PA |
| ENSG00000129675 | ARHGEF6 | Hs.522795 | 1207.5 | PP | 815.1 | PP |
| ENSG00000054267 | ARID4B | Hs.533633 | 227 | PP | 252.45 | PP |
| ENSG00000134108 | ARL10C | Hs.250009 | 578.1 | PP | 591.95 | PP |
| ENSG00000114573 | ATP6V1A | Hs.477155 | 710.1 | PP | 1105.8 | PP |
| ENSG00000155096 | AZIN1 | Hs.459106 | 551.2 | PP | 619.2 | PP |
| ENSG00000158850 | B4GALT3 | Hs.321231 | 241.5 | PP | 227.15 | PP |
| ENSG00000119866 | BCL11A | Hs.370549 | 363.1 | PP | 387.8 | PP |
| ENSG00000133639 | BTG1 | Hs.255935 | 955.9 | PP | 1057.95 | PP |
| ENSG00000159388 | BTG2 | Hs.519162 | 588.2 | PP | 538.3 | PP |
| ENSG00000132823 | C20orf111 | Hs.75798 | 346 | PP | 259.1 | PP |
| ENSG00000132424 | C6orf111 | Hs.520287 | 674.8 | PP | 743.75 | PP |
| ENSG00000111913 | C6orf32 | Hs.484915 | 83.8 | PP | 62.8 | PP |
| ENSG00000136819 | C9orf78 | Hs.278429 | 172.9 | PP | 130.25 | PP |
| ENSG00000136925 | C9orf97 | Hs.494595 | 207.5 | PP | 180.4 | PP |
| ENSG00000074410 | CA12 | Hs.210995 | 125.1 | PP | 133.8 | PP |
| ENSG00000135932 | CAB39 | Hs.471593 | 527.9 | PP | 637.3 | PP |
| ENSG00000104327 | CALB1 | Hs.65425 | 79.6 | PP | 42.05 | PA |
| ENSG00000116489 | CAPZA1 | Hs.514934 | 766.7 | PP | 1165.8 | PP |
| ENSG00000108349 | CASC3 | Hs.350229 | 286.5 | PP | 317.4 | PP |
| ENSG00000067955 | CBFB | Hs.460988 | 719.1 | PP | 1032 | PP |
| ENSG00000108468 | CBX1 | Hs.77254 | 432 | PP | 415.85 | PA |
| ENSG00000113328 | CCNG1 | Hs.79101 | 1025 | PP | 737.8 | PP |
| ENSG00000163468 | CCT3 | Hs.491494 | 1044.4 | PP | 1212.8 | PP |
| ENSG00000174059 | CD34 | Hs.374990 | 830.4 | PP | 459.3 | PP |
| ENSG00000070831 | CDC42 | Hs.467637 | 620.1 | PP | 437.75 | PP |
| ENSG00000109572 | CLCN3 | Hs.481186 | 244.9 | PP | 248.15 | PP |
| ENSG00000113240 | CLK4 | Hs.406557 | 163.3 | PP | 314.55 | PP |
| ENSG00000141367 | CLTC | Hs.491351 | 1169 | PP | 1399.6 | PP |
| ENSG00000198791 | CNOT7 | Hs.170553 | 250.3 | PP | 508.5 | PP |
| ENSG00000110880 | CORO1C | Hs.330384 | 257.4 | PP | 237.9 | PP |
| ENSG00000111605 | CPSF6 | Hs.369606 | 402.6 | PP | 474.05 | PP |
| ENSG00000005339 | CREBBP | Hs.459759 | 287.2 | PP | 239.6 | PP |
| ENSG00000099942 | CRKL | Hs.5613 | 126.4 | PP | 192.1 | PP |
| ENSG00000102974 | CTCF | Hs.368367 | 506.1 | PP | 535.55 | PP |
| ENSG00000140181 | D15F37 /// | Hs.531509 | 242.3 | PP | 427.35 | PP |
| | LOC440248 | | | | | |
| ENSG00000183283 | DAZAP2 | Hs.369761 | 1267.2 | PP | 976.7 | PP |
| ENSG00000172795 | DCP2 | Hs.443875 | 204 | PP | 270.65 | PP |
| ENSG00000124487 | DDX3X | Hs.380774 | 769.8 | PP | 1229 | PP |
| ENSG00000108654 | DDX5 | Hs.279806 | 3052 | PP | 3508.95 | PP |
| ENSG00000082458 | DLG3 | Hs.522680 | 136.2 | PP | 89.45 | PP |
| ENSG00000004975 | DVL2 | Hs.118640 | 276.9 | PP | 232.55 | PP |
| ENSG00000112242 | E2F3 | Hs.269408 | 295.6 | PP | 585.95 | PP |
| ENSG00000104517 | EDD1 | Hs.492445 | 254.6 | PP | 227.15 | PP |
| ENSG00000092847 | EIF2C1 | Hs.22867 | 292.5 | PP | 296.95 | PP |
| ENSG00000123908 | EIF2C2 | Hs.449415 | 150.2 | PP | 115.85 | PP |
| ENSG00000156976 | EIF4A2 | Hs.478553 | 3569 | PP | 6526.55 | PP |
| ENSG00000100664 | EIF5 | Hs.433702 | 96.4 | PP | 55.8 | PA |
| ENSG00000132507 | EIF5A | Hs.534314 | 1102.8 | PP | 2858.65 | PP |
| ENSG00000155849 | ELMO1 | Hs.304578 | 367.9 | PP | 339.8 | PP |
| ENSG00000012660 | ELOVL5 | Hs.520189 | 681.6 | PP | 667.3 | PP |
| ENSG00000170522 | ELOVL6 | Hs.412939 | 96.5 | PP | 156.45 | PP |
| ENSG00000182944 | EWSR1 | Hs.374477 | 389.7 | PP | 457.25 | PP |
| ENSG00000120699 | EXOSC8 | Hs.294041 | 196.9 | PP | 283.25 | PP |
| ENSG00000106462 | EZH2 | Hs.444082 | 162.9 | PP | 216.25 | PP |
| ENSG00000124491 | F13A1 | Hs.335513 | 290.7 | PP | 299.8 | PP |
| ENSG00000153310 | FAM49B | Hs.492869 | 311.6 | PP | 329.6 | PP |
| ENSG00000134884 | FLJ10154 | Hs.508644 | 843.1 | PP | 1079.5 | PP |
| ENSG00000168137 | FLJ10707 | Hs.288164 | 452.5 | PP | 256.1 | PM |
| ENSG00000159202 | FLJ13855 | Hs.514297 | 539 | PP | 525.1 | PP |
| ENSG00000153561 | FLJ13910 | Hs.75277 | 194.1 | PP | 143.15 | PP |
| ENSG00000170345 | FOS | Hs.25647 | 1254.3 | PP | 1832.65 | PP |
| ENSG00000125740 | FOSB | Hs.75678 | 1751.3 | PP | 3599.55 | PP |
| ENSG00000150907 | FOXO1A | Hs.370666 | 253.6 | PP | 140.95 | PP |
| ENSG00000114861 | FOXP1 | Hs.431498 | 322.7 | PP | 410.3 | PP |
| ENSG00000114416 | FXR1 | Hs.478407 | 143.9 | PP | 155.75 | PP |
| ENSG00000109586 | GALNT7 | Hs.127407 | 97.2 | PP | 153.95 | PP |
| ENSG00000179348 | GATA2 | Hs.367725 | 1993.1 | PP | 1452.25 | PP |
| ENSG00000135677 | GNS | Hs.334534 | 261.1 | PP | 248.15 | PP |
| ENSG00000108433 | GOSR2 | Hs.463278 | 197.9 | PP | 119.7 | PP |
| ENSG00000112414 | GPR126 | Hs.318894 | 109.5 | PP | 231.55 | PP |
| ENSG00000103342 | GSPT1 | Hs.528780 | 356.7 | PP | 369.6 | PP |
| ENSG00000140307 | GTF2A2 | Hs.512934 | 276 | PP | 270.75 | PP |
| ENSG00000164032 | H2AFZ | Hs.119192 | 1477 | PP | 3477.65 | PP |
| ENSG00000122566 | HNRPA2B1 | Hs.487774 | 1802.2 | PP | 2268.3 | PP |
| ENSG00000125944 | HNRPR | Hs.373763 | 839.4 | PP | 980.1 | PP |
| ENSG00000153187 | HNRPU | Hs.166463 | 470.3 | PP | 887.6 | PP |
| ENSG00000105323 | HNRPUL1 | Hs.155218 | 406.9 | PP | 401.15 | PP |
| ENSG00000153807 | HOXA10 | Hs.110637 | 161.3 | PP | 200.7 | PA |
| ENSG00000078399 | HOXA9 | Hs.127428 | 739.2 | PP | 674 | PP |
| ENSG00000173744 | HRB | Hs.443507 | 202.6 | PP | 405.7 | PP |
| ENSG00000074356 | HSA277841 | Hs.120963 | 222.9 | PP | 150.05 | PP |
| ENSG00000144381 | HSPD1 | Hs.113684 | 2868.6 | PP | 5689.3 | PP |
| ENSG00000171105 | INSR | Hs.465744 | 247.7 | PP | 292.45 | PP |
| ENSG00000184916 | JAG2 | Hs.433445 | 261.3 | PP | 227.15 | PP |
| ENSG00000115548 | JMJD1A | Hs.531819 | 264 | PP | 207.05 | PP |
| ENSG00000171988 | JMJD1C | Hs.413416 | 107 | PP | 198.85 | PP |
| ENSG00000197226 | KIAA0676 | Hs.155829 | 362.1 | PP | 398.15 | PP |
| ENSG00000170759 | KIF5B | Hs.327736 | 346.6 | PP | 430.75 | PP |
| ENSG00000136826 | KLF4 | Hs.376206 | 536.5 | PP | 1193.7 | PP |
| ENSG00000102753 | KPNA3 | Hs.527919 | 250.8 | PP | 391.35 | PP |
| ENSG00000189067 | LITAF | Hs.459940 | 725.1 | PP | 513.5 | PP |
| ENSG00000124831 | LRRFIP1 | Hs.471779 | 429.8 | PP | 408.25 | PP |
| ENSG00000175324 | LSM1 | Hs.425311 | 378.1 | PP | 497.8 | PP |
| ENSG00000121579 | MAK3 | Hs.269528 | 268.8 | PP | 307 | PP |
| ENSG00000012983 | MAP4K5 | Hs.130491 | 66 | PP | 116 | PP |
| ENSG00000112062 | MAPK14 | Hs.485233 | 507.4 | PP | 486.3 | PP |
| ENSG00000082212 | ME2 | Hs.233119 | 471.1 | PP | 513.15 | PP |
| ENSG00000147649 | MTDH | Hs.377155 | 258.6 | PP | 329.8 | PP |
| ENSG00000108389 | MTMR4 | Hs.514373 | 330.7 | PP | 407.2 | PP |
| ENSG00000170873 | MTSS1 | Hs.336994 | 152.7 | PP | 194.6 | PP |
| ENSG00000136504 | MYST2 | Hs.21907 | 331.7 | PP | 192.25 | PP |
| ENSG00000138386 | NAB1 | Hs.107474 | 187.9 | PP | 203.9 | PP |
| ENSG00000164258 | NDUFS4 | Hs.528222 | 383.2 | PP | 290.65 | PP |
| ENSG00000069869 | NEDD4 | Hs.1565 | 186.1 | PP | 193.9 | PP |
| ENSG00000082641 | NFE2L1 | Hs.514284 | 735.5 | PP | 726.35 | PP |
| ENSG00000116044 | NFE2L2 | Hs.155396 | 181.2 | PP | 280.9 | PP |
| ENSG00000166681 | NGFRAP1 | Hs.448588 | 2654.4 | PP | 3641.6 | PP |
| ENSG00000164190 | NIPBL | Hs.481927 | 165.1 | PP | 145.15 | PP |
| ENSG00000180530 | NRIP1 | Hs.155017 | 842 | PP | 914.85 | PP |
| ENSG00000124789 | NUP153 | Hs.121088 | 216.4 | PP | 291.8 | PP |
| ENSG00000162231 | NXF1 | Hs.523739 | 543 | PP | 626 | PP |
| ENSG00000101888 | NXT2 | Hs.25010 | 135.9 | PP | 271.1 | PP |
| ENSG00000046651 | OFD1 | Hs.6483 | 1022.7 | PP | 692.15 | PP |
| ENSG00000172939 | OXSR1 | Hs.475970 | 211 | PP | 186.15 | PA |
| ENSG00000070756 | PABPC1 | Hs.387804 | 7565 | PP | 7921.7 | PP |
| ENSG00000151846 | PABPC3 | Hs.458280 | 4002.7 | PP | 3510.25 | PP |
| ENSG00000100836 | PABPN1 | Hs.117176 | 758.2 | PP | 790.85 | PP |
| ENSG00000157654 | PALM2- | Hs.259461 | 181.4 | PP | 163.75 | PP |
| | AKAP2 | | | | | |
| ENSG00000145730 | PAM | Hs.369430 | 187.3 | PP | 178.45 | PP |
| ENSG00000168283 | PCGF4 | Hs.380403 | 254.7 | PP | 304.1 | PP |
| ENSG00000184588 | PDE4B | Hs.198072 | 234.1 | PP | 349.5 | PP |
| ENSG00000065485 | PDIA5 | Hs.477352 | 352.8 | PP | 418.95 | PP |
| ENSG00000152556 | PFKM | Hs.75160 | 303.1 | PP | 332 | PP |
| ENSG00000167085 | PHB | Hs.514303 | 723.7 | PP | 649.4 | PP |
| ENSG00000145675 | PIK3R1 | Hs.132225 | 249.1 | PP | 181 | PP |
| ENSG00000171033 | PKIA | Hs.433700 | 88.6 | PP | 131.05 | PP |
| ENSG00000136040 | PLXNC1 | Hs.551522 | 354.6 | PP | 387.1 | PP |
| ENSG00000112941 | POLS | Hs.481542 | 355.5 | PP | 367.35 | PP |
| ENSG00000163806 | PPP1CB | Hs.468018 | 65.7 | PP | 135.05 | PP |
| ENSG00000163806 | PPP1CB | Hs.468018 | 140 | PP | 176 | PP |
| ENSG00000186298 | PPP1CC | Hs.79081 | 709.2 | PP | 1286.15 | PP |
| ENSG00000104762 | PPP2R2A | Hs.146339 | 331.7 | PP | 299.6 | PP |
| ENSG00000107758 | PPP3CB | Hs.500067 | 318.9 | PP | 241.25 | PP |
| ENSG00000166501 | PRKCB1 | Hs.460355 | 742.5 | PP | 535.05 | PP |
| ENSG00000129084 | PSMA1 | Hs.102798 | 1702 | PP | 1305.3 | PP |
| ENSG00000171522 | PTGER4 | Hs.199248 | 531.8 | PP | 514.8 | PP |
| ENSG00000112531 | QKI | Hs.510324 | 284.1 | PP | 564.45 | PP |
| ENSG00000103769 | RAB11A | Hs.321541 | 1087.7 | PP | 1020.15 | PP |
| ENSG00000080371 | RAB21 | Hs.524590 | 103.7 | PP | 212.9 | PP |
| ENSG00000111737 | RAB35 | Hs.524788 | 394.6 | PP | 431.2 | PP |
| ENSG00000167461 | RAB8A | Hs.5947 | 675.2 | PP | 756.75 | PP |
| ENSG00000127314 | RAP1B | Hs.369920 | 1093.1 | PP | 1537.75 | PP |
| ENSG00000109756 | RAPGEF2 | Hs.113912 | 394.9 | PP | 551.95 | PP |
| ENSG00000142599 | RERE | Hs.463041 | 480.2 | PP | 252.15 | PP |
| ENSG00000056687 | RGL2 | Hs.509622 | 340.5 | PP | 232.1 | PA |
| ENSG00000090104 | RGS1 | Hs.75256 | 90.4 | PP | 99.4 | PP |
| ENSG00000106615 | RHEB | Hs.283521 | 943.5 | PP | 1172.5 | PP |
| ENSG00000067560 | RHOA | Hs.247077 | 1537.7 | PP | 2368.8 | PP |
| ENSG00000126858 | RHOT1 | Hs.462742 | 196.4 | PP | 304.85 | PP |
| ENSG00000170881 | RNF139 | Hs.492751 | 392.9 | PP | 435.75 | PP |
| ENSG00000137075 | RNF38 | Hs.333503 | 322.6 | PP | 227.7 | PP |
| ENSG00000043093 | RP42 | Hs.104613 | 236.5 | PP | 204.45 | PP |
| ENSG00000163902 | RPN1 | Hs.518244 | 291.2 | PP | 302.5 | PA |
| ENSG00000100784 | RPS6KA5 | Hs.510225 | 152.1 | PP | 132.75 | PP |
| ENSG00000144580 | RQCD1 | Hs.148767 | 284.7 | PP | 361.25 | PP |
| ENSG00000115310 | RTN4 | Hs.429581 | 710.8 | PP | 1341.5 | PP |
| ENSG00000159216 | RUNX1 | Hs.149261 | 108.8 | PP | 211.2 | PP |
| ENSG00000163602 | RYBP | Hs.7910 | 398 | PP | 462.8 | PP |
| ENSG00000182568 | SATB1 | Hs.517717 | 829.9 | PP | 652.05 | PP |
| ENSG00000092421 | SEMA6A | Hs.156967 | 140 | PP | 182.2 | PP |
| ENSG00000179833 | SERTAD2 | Hs.77293 | 498.4 | PP | 556.2 | PP |
| ENSG00000119335 | SET | Hs.436687 | 2967 | PP | 4307.1 | PP |
| ENSG00000116560 | SFPQ | Hs.355934 | 306.9 | PP | 689 | PP |
| ENSG00000136450 | SFRS1 | Hs.68714 | 1264.9 | PP | 1622.65 | PP |
| ENSG00000153914 | SFRS12 | Hs.519347 | 258 | PP | 229.6 | PP |
| ENSG00000139218 | SFRS2IP | Hs.210367 | 224.1 | PP | 132.2 | PA |
| ENSG00000108061 | SHOC2 | Hs.104315 | 283.3 | PP | 262.55 | PP |
| ENSG00000096717 | SIRT1 | Hs.369779 | 133.8 | PP | 124.9 | PP |
| ENSG00000147454 | SLC25A37 | Hs.122514 | 103 | PP | 86.95 | PA |
| ENSG00000112305 | SMAP1 | Hs.485717 | 205.2 | PP | 129.15 | PP |
| ENSG00000080503 | SMARCA2 | Hs.298990 | 264.4 | PP | 201.3 | PP |
| ENSG00000084090 | STARD7 | Hs.469331 | 1166.2 | PP | 1442.7 | PP |
| ENSG00000102572 | STK24 | Hs.508514 | 958.4 | PP | 1363.75 | PP |
| ENSG00000198648 | STK39 | Hs.276271 | 129.8 | PP | 111.35 | PP |
| ENSG00000124222 | STX16 | Hs.307913 | 474.2 | PP | 228.9 | PP |
| ENSG00000116030 | SUMO1 | Hs.81424 | 240.9 | PP | 268.85 | PP |
| ENSG00000178691 | SUZ12 | Hs.462732 | 602.2 | PP | 633.3 | PP |
| ENSG00000008282 | SYPL1 | Hs.80919 | 157.3 | PP | 190.55 | PP |
| ENSG00000135090 | TAOK3 | — | 518.8 | PP | 394 | PP |
| ENSG00000177426 | TGIF | Hs.373550 | 116.7 | PP | 95.35 | PM |
| ENSG00000123066 | THRAP2 | Hs.159799 | 142.1 | PP | 396.2 | PP |
| ENSG00000116001 | TIA1 | Hs.516075 | 151.6 | PP | 145 | PP |
| ENSG00000101337 | TM9SF4 | Hs.529360 | 129.2 | PP | 121.3 | PP |
| ENSG00000034510 | TMSB10 | Hs.446574 | 8602.5 | PP | 6478.75 | PP |
| ENSG00000083312 | TNPO1 | Hs.482497 | 483 | PP | 750.85 | PP |
| ENSG00000064419 | TNPO3 | Hs.193613 | 392.6 | PP | 422.55 | PP |
| ENSG00000173334 | TRIB1 | Hs.444947 | 238.3 | PP | 300.85 | PP |
| ENSG00000116747 | TROVE2 | Hs.288178 | 480.7 | PP | 473.25 | PP |
| ENSG00000165699 | TSC1 | Hs.370854 | 232.7 | PP | 242.3 | PP |
| ENSG00000139921 | TXNDC | Hs.125221 | 200.8 | PP | 393.7 | PP |
| ENSG00000170035 | UBE2E3 | Hs.470804 | 311.5 | PP | 258.25 | PP |
| ENSG00000177889 | UBE2N | Hs.524630 | 415.5 | PP | 551.55 | PP |
| ENSG00000160714 | UBE2Q | Hs.516587 | 457.1 | PP | 377.95 | PP |
| ENSG00000124208 | UBE2V1/// | Hs.420529 | 504.3 | PP | 320.6 | PP |
| | Kua-UEV | | | | | |
| ENSG00000110344 | UBE4A | Hs.75275 | 271.3 | PP | 404.8 | PP |
| ENSG00000115464 | USP34 | Hs.468663 | 1075.5 | PP | 1012.75 | PP |
| ENSG00000124486 | USP9X | Hs.77578 | 85.3 | PP | 115.1 | PP |
| ENSG00000035403 | VCL | Hs.500101 | 332.1 | PP | 541.1 | PP |
| ENSG00000119541 | VPS4B | Hs.126550 | 305.9 | PP | 385.15 | PP |
| ENSG00000132716 | WDR42A | Hs.492236 | 195.9 | PP | 233.1 | PP |
| ENSG00000109046 | WSB1 | Hs.446017 | 679.3 | PP | 880.75 | PP |
| ENSG00000169180 | XPO6 | Hs.460468 | 395.3 | PP | 515.4 | PP |
| ENSG00000166913 | YWHAB | Hs.279920 | 222.4 | PP | 288.75 | PP |
| ENSG00000134308 | YWHAQ | Hs.74405 | 922.3 | PP | 2337.65 | PP |
| ENSG00000186908 | ZDHHC17 | Hs.4014 | 123.5 | PP | 166.2 | PP |
| ENSG00000185650 | ZFP36L1 | Hs.85155 | 94.5 | PP | 140.7 | PP |
| ENSG00000010244 | ZNF207 | Hs.500775 | 918.9 | PP | 742.3 | PP |
| ENSG00000179456 | ZNF238 | Hs.69997 | 235.7 | PP | 241.8 | PP |
| ENSG00000172466 | ZNF24 | Hs.514802 | 240.6 | PP | 213.65 | PP |
| ENSG00000036549 | ZZZ3 | Hs.480506 | 125 | PP | 189.7 | PP |

212

| | | | | | | |
|---|---|---|---|---|---|---|
| ENSG00000138031 | ADCY3 | Hs.467898 | 208.6 | PP | 178.4 | PP |
| ENSG00000140350 | ANP32A | Hs.458747 | 416 | PP | 331.55 | PP |
| ENSG00000062725 | APPBP2 | Hs.84084 | 106.4 | PP | 116.15 | PA |
| ENSG00000117713 | ARID1A | Hs.468972 | 477.6 | PP | 444.8 | PP |
| ENSG00000155096 | AZIN1 | Hs.459106 | 551.2 | PP | 619.2 | PP |
| ENSG00000133935 | C14orf1 | Hs.15106 | 292.6 | PP | 249.85 | PP |
| ENSG00000153113 | CAST | Hs.440961 | 301.7 | PP | 461.25 | PP |
| ENSG00000108468 | CBX1 | Hs.77254 | 432 | PP | 415.85 | PP |
| ENSG00000100528 | CNIH | Hs.294603 | 252.6 | PP | 391.7 | PP |
| ENSG00000160967 | CUTL1 | Hs.438974 | 333.3 | PP | 412.55 | PP |
| ENSG00000183283 | DAZAP2 | Hs.369761 | 1267.2 | PP | 976.7 | PP |
| ENSG00000156976 | EIF4A2 | Hs.478553 | 3569 | PP | 6526.55 | PP |
| ENSG00000104064 | GABPB2 | Hs.546852 | 129.3 | PP | 183.75 | PM |
| ENSG00000141429 | GALNT1 | Hs.514806 | 220 | PP | 273.8 | PP |
| ENSG00000106070 | GRB10 | Hs.164060 | 550.7 | PP | 574.15 | PP |
| ENSG00000164032 | H2AFZ | Hs.119192 | 1477 | PP | 3477.65 | PP |
| ENSG00000100281 | HMG2L1 | Hs.197086 | 215.2 | PP | 87.75 | PM |
| ENSG00000099783 | HNRPM | Hs.465808 | 968.5 | PP | 880.2 | PP |
| ENSG00000153187 | HNRPU | Hs.166463 | 470.3 | PP | 887.6 | PP |
| ENSG00000010295 | HOM-TES- | Hs.15243 | 407 | PP | 355.35 | PP |
| | 103 | | | | | |
| ENSG00000078399 | HOXA9 | Hs.127428 | 739.2 | PP | 674 | PP |
| ENSG00000108622 | ICAM2 | Hs.431460 | 319.9 | PP | 269.45 | PP |
| ENSG00000135387 | M11S1 | Hs.471818 | 464.9 | PP | 451.45 | PP |
| ENSG00000101367 | MAPRE1 | Hs.472437 | 196.1 | PP | 206.85 | PM |
| ENSG00000168906 | MAT2A | Hs.516157 | 1011.4 | PP | 738.6 | PP |
| ENSG00000152601 | MBNL1 | Hs.478000 | 294.4 | PP | 864.5 | PA |
| ENSG00000081189 | MEF2C | Hs.444409 | 426 | PP | 437.85 | PP |
| ENSG00000143995 | MEIS1 | Hs.526754 | 452.1 | PP | 262.65 | PP |
| ENSG00000108389 | MTMR4 | Hs.514373 | 330.7 | PP | 407.2 | PP |
| ENSG00000133026 | MYH10 | Hs.16355 | 375.1 | PP | 362.5 | PP |
| ENSG00000153234 | NR4A2 | Hs.165258 | 124.6 | PP | 242.85 | PP |
| ENSG00000145730 | PAM | Hs.369430 | 187.3 | PP | 178.45 | PP |
| ENSG00000136040 | PLXNC1 | Hs.551522 | 354.6 | PP | 387.1 | PP |
| ENSG00000100941 | PNN | Hs.409965 | 839.5 | PP | 617.3 | PP |
| ENSG00000078304 | PPP2R5C | Hs.368264 | 174.9 | PP | 165.25 | PP |
| ENSG00000127314 | RAP1B | Hs.369920 | 1093.1 | PP | 1537.75 | PP |
| ENSG00000181467 | RAP2B | Hs.98643 | 583.1 | PP | 665.95 | PP |
| ENSG00000125976 | RBM12 | — | 321.1 | PP | 313.35 | PP |
| ENSG00000137710 | RDX | Hs.263671 | 189.5 | PP | 184.9 | PP |
| ENSG00000137075 | RNF38 | Hs.333503 | 322.6 | PP | 227.7 | PP |
| ENSG00000137575 | SDCBP | Hs.200804 | 934 | PP | 955.25 | PP |
| ENSG00000122545 | 7-Sep | Hs.191346 | 815 | PP | 1146.5 | PP |
| ENSG00000136450 | SFRS1 | Hs.68714 | 1264.9 | PP | 1622.65 | PP |
| ENSG00000096717 | SIRT1 | Hs.369779 | 133.8 | PP | 124.9 | PP |
| ENSG00000117394 | SLC2A1 | Hs.473721 | 233.5 | PP | 290.75 | PP |
| ENSG00000124766 | SOX4 | Hs.357901 | 2351.7 | PP | 2500.65 | PP |
| ENSG00000091640 | SPAG7 | Hs.90436 | 900.7 | PP | 517.9 | PP |
| ENSG00000164056 | SPRY1 | Hs.436944 | 360.7 | PP | 268.15 | PP |
| ENSG00000157514 | TSC22D3 | Hs.522074 | 1752.5 | PP | 2189.45 | PP |
| ENSG00000163084 | TSN | Hs.75066 | 130.8 | PP | 178.35 | PP |
| ENSG00000116809 | ZBTB17 | Hs.433764 | 249.3 | PP | 146.75 | PP |
| ENSG00000114514 | ZNF148 | Hs.380334 | 211.6 | PP | 226.65 | PP |

32

| | | | | | | |
|---|---|---|---|---|---|---|
| ENSG00000138031 | ADCY3 | Hs.467898 | 208.6 | PP | 178.4 | PP |
| ENSG00000143401 | ANP32E | Hs.385913 | 169.2 | PP | 423.3 | PP |
| ENSG00000117713 | ARID1A | Hs.468972 | 477.6 | PP | 444.8 | PP |
| ENSG00000174437 | ATP2A2 | Hs.506759 | 203.5 | PP | 255.4 | PP |
| ENSG00000155096 | AZIN1 | Hs.459106 | 551.2 | PP | 619.2 | PP |
| ENSG00000159388 | BTG2 | Hs.519162 | 588.2 | PP | 538.3 | PP |
| ENSG00000112237 | CCNC | Hs.430646 | 273.3 | PP | 380.8 | PP |
| ENSG00000070831 | CDC42 | Hs.467637 | 620.1 | PP | 437.75 | PP |
| ENSG00000118260 | CREB1 | Hs.516646 | 122.9 | PP | 230.35 | PP |
| ENSG00000082458 | DLG3 | Hs.522680 | 136.2 | PP | 89.45 | PP |
| ENSG00000128590 | DNAJB9 | Hs.6790 | 211.5 | PP | 433.55 | PP |
| ENSG00000112242 | E2F3 | Hs.269408 | 295.6 | PP | 585.95 | PP |
| ENSG00000120738 | EGR1 | Hs.326035 | 128.6 | PP | 290.15 | PP |
| ENSG00000104131 | EIF3S1 | Hs.404056 | 340.4 | PP | 365.05 | PP |
| ENSG00000110321 | EIF4G2 | Hs.183684 | 2137 | PP | 2701.9 | PP |
| ENSG00000100664 | EIF5 | Hs.433702 | 96.4 | PP | 55.8 | PA |
| ENSG00000114416 | FXR1 | Hs.478407 | 143.9 | PP | 155.75 | PP |
| ENSG00000138757 | G3BP2 | Hs.303676 | 242.8 | PP | 428.55 | PP |
| ENSG00000106070 | GRB10 | Hs.164060 | 550.7 | PP | 574.15 | PP |
| ENSG00000172534 | HCFC1 | Hs.83634 | 146.2 | PP | 233.9 | PA |
| ENSG00000173744 | HRB | Hs.443507 | 202.6 | PP | 405.7 | PP |
| ENSG00000144381 | HSPD1 | Hs.113684 | 2868.6 | PP | 5689.3 | PP |
| ENSG00000143164 | IQWD1 | Hs.435741 | 168.5 | PP | 156.4 | PP |
| ENSG00000150093 | ITGB1 | Hs.429052 | 865.6 | PP | 593.85 | PP |
| ENSG00000117139 | JARID1B | Hs.443650 | 203.6 | PP | 353.6 | PP |
| ENSG00000136826 | KLF4 | Hs.376206 | 536.5 | PP | 1193.7 | PA |
| ENSG00000110514 | MADD | Hs.82548 | 423 | PP | 267.9 | PA |
| ENSG00000175130 | MARCKSL1 | Hs.75061 | 314.8 | PP | 265.6 | PP |
| ENSG00000180398 | MCFD2 | Hs.293689 | 289.4 | PP | 258.25 | PP |
| ENSG00000081189 | MEF2C | Hs.444409 | 426 | PP | 437.85 | PP |
| ENSG00000005810 | MYCBP2 | Hs.151411 | 187 | PP | 643.55 | PP |
| ENSG00000106459 | NRF1 | Hs.298069 | 157.5 | PP | 126.8 | PA |
| ENSG00000170515 | PA2G4 | Hs.478044 | 251.3 | PP | 523.75 | PP |
| ENSG00000070756 | PABPC1 | Hs.387804 | 7565 | PP | 7921.7 | PP |
| ENSG00000145730 | PAM | Hs.369430 | 187.3 | PP | 178.45 | PP |
| ENSG00000090060 | PAPOLA | Hs.253726 | 307.4 | PP | 286 | PP |
| ENSG00000185619 | PCGF3 | Hs.144309 | 192.1 | PP | 237.55 | PP |
| ENSG00000102225 | PCTK1 | Hs.496068 | 135 | PP | 88.45 | PA |
| ENSG00000025293 | PHF20 | Hs.517044 | 249.8 | PP | 173.65 | PP |
| ENSG00000112941 | POLS | Hs.481542 | 355.5 | PP | 367.35 | PP |
| ENSG00000180817 | PP | Hs.437403 | 894.8 | PP | 1860.7 | PP |
| ENSG00000100614 | PPM1A | Hs.130036 | 197.3 | PP | 259.8 | PP |
| ENSG00000163806 | PPP1CB | Hs.468018 | 65.7 | PP | 135.05 | PP |
| ENSG00000171522 | PTGER4 | Hs.199248 | 531.8 | PP | 514.8 | PP |
| ENSG00000048991 | R3HDM | Hs.412462 | 217.6 | PP | 376.75 | PP |
| ENSG00000127314 | RAP1B | Hs.369920 | 1093.1 | PP | 1537.75 | PP |
| ENSG00000067560 | RHOA | Hs.247077 | 1537.7 | PP | 2368.8 | PP |
| ENSG00000119729 | RHOQ | Hs.549125 | 187.1 | PP | 222.3 | PP |
| ENSG00000134758 | RNF138 | Hs.302408 | 186.9 | PP | 511.9 | PP |
| ENSG00000137075 | RNF38 | Hs.333503 | 322.6 | PP | 227.7 | PP |
| ENSG00000182568 | SATB1 | Hs.517717 | 829.9 | PP | 652.05 | PP |
| ENSG00000156642 | SDFR1 | Hs.187866 | 169.4 | PP | 193.05 | PP |
| ENSG00000116754 | SFRS11 | Hs.479693 | 819.9 | PP | 1077.55 | PP |
| ENSG00000153914 | SFRS12 | Hs.519347 | 258 | PP | 229.6 | PP |
| ENSG00000139218 | SFRS2IP | Hs.210367 | 224.1 | PP | 132.2 | PA |
| ENSG00000096717 | SIRT1 | Hs.369779 | 133.8 | PP | 124.9 | PP |
| ENSG00000184602 | SNN | Hs.459952 | 234.4 | PP | 239.3 | PP |
| ENSG00000159140 | SON | Hs.517262 | 408.8 | PP | 467.15 | PP |
| ENSG00000137642 | SORL1 | Hs.368592 | 2264.1 | PP | 1084.65 | PP |
| ENSG00000124766 | SOX4 | Hs.357901 | 2351.7 | PP | 2500.65 | PP |
| ENSG00000172845 | SP3 | Hs.531587 | 137.8 | PP | 293.35 | PP |
| ENSG00000178691 | SUZ12 | Hs.462732 | 602.2 | PP | 633.3 | PP |
| ENSG00000165169 | TCTE1L | Hs.446392 | 323 | PP | 369.75 | PP |
| ENSG00000116001 | TIA1 | Hs.516075 | 151.6 | PP | 145 | PP |
| ENSG00000183864 | TOB2 | Hs.474978 | 165.3 | PP | 196.4 | PP |
| ENSG00000154174 | TOMM70A | Hs.227253 | 585.1 | PP | 581.85 | PP |
| ENSG00000170142 | UBE2E1 | Hs.164853 | 318.4 | PP | 488.3 | PP |
| ENSG00000164828 | UNC84A | Hs.438072 | 146.4 | PP | 111.15 | PP |
| ENSG00000135655 | USP15 | Hs.434951 | 197.3 | PP | 160.35 | PP |
| ENSG00000143952 | VPS54 | Hs.48499 | 187.9 | PP | 151.5 | PP |
| ENSG00000179456 | ZNF238 | Hs.69997 | 235.7 | PP | 241.8 | PP |

100

| | | | | | | |
|---|---|---|---|---|---|---|
| ENSG00000123908 | EIF2C2 | Hs.449415 | 150.2 | PP | 115.85 | PP |
| ENSG00000022267 | FHL1 | Hs.435369 | 5240 | PP | 3151.75 | PP |
| ENSG00000153187 | HNRPU | Hs.166463 | 470.3 | PP | 887.6 | PP |
| ENSG00000152601 | MBNL1 | Hs.478000 | 294.4 | PP | 864.5 | PP |
| ENSG00000100330 | MTMR3 | Hs.474536 | 204.7 | PP | 287 | PP |
| ENSG00000082641 | NFE2L1 | Hs.514284 | 735.5 | PP | 726.35 | PP |
| ENSG00000136448 | NMT1 | Hs.532790 | 220.5 | PP | 219.45 | PP |
| ENSG00000147162 | OGT | Hs.405410 | 165.1 | PP | 183.6 | PP |
| ENSG00000136238 | RAC1 | Hs.413812 | 767.5 | PP | 1337.4 | PP |
| ENSG00000092421 | SEMA6A | Hs.156967 | 140 | PP | 182.2 | PP |

### Example 4

### MicroRNA-16 Inhibits Human Erythroid Differentiation

### Abstract

Previously, the inventors determined that microRNA-16 (miR-16) is expressed in normal human CD34⁺ hematopoietic stem-progenitor cells (HSPCs) and predicted by bio-informatics that miR-16 may regulate erythroid differentiation. Here the inventors report that ectopic miR-16 expression completely blocked hemin-induced erythroid differentiation of K562 human erythroleukemia cells. Likewise, enforced miR-16 expression in normal human CD34⁺ cells strongly inhibited erythroid differentiation, without affecting myeloid differentiation in either colony-forming or suspension culture assays. This study provides functional evidence verifying the inventors' bioinformatic prediction that miR-16 can selectively regulate human erythroid differentiation.

### Introduction

MicroRNAs (miRs) are ˜22 nucleotide RNAs that post-transcriptionally regulate mRNA molecules¹ and can thereby control cellular proliferation, differentiation and metabolism². The chromosome 13q14 locus of miR-15/16, the first microRNA described as a tumor suppressor gene, is deleted in many human chronic lymphoid leukemia (CLL) cases³, a finding replicated for the syntenic deletion in mice that develop CLL⁴. In normal B lymphoid cells, miR-16 down-regulates the level of BCL-2 protein, leading to increased cellular susceptibility to apoptosis⁵. In addition, the miR-16 family has been bio-informatically suggested to down-regulate cell cycle-associated mRNAs⁶. Recently, miR-16 has been implicated in Xenopus cellular differentiation via controlling Nodal signaling⁷. In mammalian cellular differentiation, miR-16 expression increases during terminal erythroid maturation⁸.

In a previous study, the inventors determined miR expression in normal human CD34⁺ HSPCs⁹ and combined this with HSPC mRNA expression data¹⁰ to bio-informatically predict possible roles that miRs play in hematopoiesis, with a particular focus on possible control points in stem to progenitor cell transitions. This analysis identified the possible involvement of miR-16 in either myeloid or erythroid differentiation. In the current study, the inventors doc Ument dynamic miR-16 expression during myeloid and erythroid differentiation, and show by functional assays that miR-16 expression markedly decreases erythropoiesis in the K562 cell line model and in normal primary human CD34⁺ HSPCs without affecting myelopoiesis.

### Materials and Methods

### Cell Culture

K562 (ATCC, Rockville, Md.) were cultured, and CD34⁺ cells from normal adult donor mobilized blood were obtained from the National Heart, Lung, and Blood Institute Program of Excellence in Gene Therapy, Hematopoietic Cell Processing Core (Fred Hutchison Cancer Center) under Institutional Review Board-approved protocols of both the Fred Hutchinson Cancer Center and the Johns Hopkins School of Medicine, and cultured as previously described^{9,10}.

Lentivirus Production and miR-16 Ectopic Expression

The 630 by genomic sequence surrounding hsa-miR-16a was inserted into dual promoter FUGW parental vector containing green fluorescent protein (GFP)^{11,12}. CD34⁺ cells were transduced with miR-16 or parental control lentivirus as previously described⁹.

### MicroRNA Isolation and Northern Blotting

Erythroid differentiation of K562 cells was induced with 50 ng/ml Hemin^{—}. Cells were harvested over a 7 day time course, and total RNA was isolated with TriZol (Invitrogen, Carlsbad, Calif.). MiR-16a expression was determined by miR-microarray as previously described⁹, and verified by Northern blotting.

### SILAC Proteomic Assay

Control and miR-16-transduced K562 cells were treated with hemin or vehicle control. At day 7 (d7), cellular protein was isolated from 2×10⁷ cells and assayed by Stable Isotope Labeling with Amino Acids in Cell Culture (SILAC) proteomics as described^{14,15}.

### Methycellulose Colony-Forming and Suspension Culture Assays

Lentivirally transduced CD34⁺ cells were fluorescence activated cell sorter (FACS)-sorted two days after transduction for GFP⁺cells using a FACS Vantage (BD Biosciences, San Jose, CA) flow cytometer. GFP⁺ cells were plated into colony-forming cell (CFC) and suspension culture assays as previously described⁹. CFC-GM and BFU-E colonies from CFC assays were enumerated 14 days after transduction. Cells from suspension cultures were immunostained 7 days after FACS sorting with CD33, CD34, CD71, CD235a and isotype control monoclonal antibodies (BD).

### Results and Discussion

### MiR-16 Decreased During K562 Cell Erythroid Differentiation

The inventors' previous miR expression and bio-informatic studies implicated miR-16 as a possible regulator of human myel- and/or erythropoiesis, because of its multiple predicted hematopoietic-expressed mRNA targets, including MEIS1, CREBBP, CD164, HOXA5, and AGTR2⁹. First, the inventors examined miR-16 expression during a time course of hemin-induced K562 model erythroid differentiation and HL-60 model differentiation to multiple terminal cell types. MiR-16 was expressed in both untreated K562 and HL-60 cells. During hemin-induced erythroid differentiation, mir-16 levels increased slightly at very early time points (1-4 hrs), then decreased to low levels at d1. (FIG. 9A) Expression remained low until d7, when the cells were fully differentiated and mir-16 returned to the level present in uninduced K562. In contrast, miR-16 levels did not change in HL60 cells treated with any of 4 different inducers of myeloid differentiation. (FIG. 9B) These expression kinetics indicated that miR-16 may selectively hold erythroid, but not myeloid, progenitor cells at an undifferentiated state. Up-regulation of miR-16 was previously observed in erythroid differentiation, and its expression correlated with the appearance of erythroid cell surface markers such as CD71, CD36, and CD235a⁸. The inventors' expression results confirm that miR-16 expression is higher in fully differentiated cells, and extends the prior study by revealing that mir-16 expression is highly dynamic during erythroid differentiation. Based on this dynamic expression during erythropoiesis, physiologic miR-16 reduction may allow cells to differentiate along the erythroid lineage.

To determine if sustained miR-16 can down-regulate erythropoiesis, the inventors transduced K562 cells with miR-16 and found that benzidine staining (of hemoglobin) was decreased by 78% (data not shown). Furthermore, SILAC proteomics showed that, compared to hemin-treated, control-transduced K562 cells, miR-16-transduced, hemin-treated K562 cells had greatly decreased levels of erythrocyte-associated proteins, including the globins. (FIG. 9C) Enforced expression of miR-16 did not change either cellular apoptosis (assayed by viaprobe/annexin V staining), cell cycling (assayed by propidium iodide staining), or cell growth (assayed by cell counts) (data not shown).

Thus, preventing the physiologic reduction in miR-16 by enforced expression blocked hemin-induced K562 erythroid differentiation.

### Mir-16 Suppressed Erythropoiesis of Normal CD34+ HSPC.

The inventors next hypothesized that enforced miR-16 expression would selectively inhibit erythropoiesis of primary human HSPCs. Indeed, miR-16-transduced CD34⁺ cells generated 30% fewer erythroid colonies but only 10% fewer myeloid colonies. (FIG. 10a) In addition, the erythroid colonies generated by miR-16-transduced CD34⁺ cells were small and diffuse compared to controls, while the myeloid colonies were normal in size and morphology (not shown). In suspension cultures, cells ectopically expressing miR-16 generated 3-fold fewer CD71⁺/CD235a⁺ relatively early erythroid cells and 4-fold fewer later CD71⁺⁺/CD235a⁺⁺ erythroid cells ¹⁶. (FIG. 10b) In contrast, there was a 30% increase in the frequency of CD33⁺ myeloid cells in the suspension cultures of miR-16-transduced cells, confirming the CFC data that myeloid differentiation was relatively insensitive to mir-16 expression. (FIG. 10b) Although miR-16 can regulate apoptosis in B lymphoid cells^{3,5} and has also been suggested as a cell cycle inhibitor⁶, the inventors found that apoptosis, cell cycling, and cell counts of mir-16-transduced CD34⁺ cells in suspension cultures were comparable to controls (data not shown).

The inventors' original study of miR expression in HSPCs identified miR-16 as a hematopoietic expressed (HE)-miR, and target annotation indicated it as a possible regulator of erythropoiesis⁹. The inventors also showed that several mRNAs predicted to be miR-16 targets are in fact translationally controlled in hematopoietic cells⁹. In the functional studies herein, the inventors have demonstrated that miR-16 can potently and selectively down-regulate erythropoiesis of both K562 erythroleukemia cells and normal primary CD34⁺ HSPCs.

### REFERENCES

1. Ambros V, Chen X. The regulation of genes and genomes by small RNAs. Development. 2007;134:1635-1641. 2. Bartel D P. MicroRNAs: genomics, biogenesis, mechanism, and function. Cell. 2004;116:281-297. 3. Calin G A, Dumitru C D, Shimizu M, et al. Frequent deletions and down-regulation of micro-RNA genes miR15 and miR16 at 13q14 in chronic lymphocytic leukemia. Proc Natl Acad Sci USA. 2002;99:15524-15529. 4. Raveche E S, Salerno E, Scaglione B J, et al. Abnormal microRNA-16 locus with synteny to human 13q14 linked to CLL in NZB mice. Blood. 2007;109:5079-5086. 5. Cimmino A, Calin G A, Fabbri M, et al. miR-15 and miR-16 induce apoptosis by targeting BCL2. Proc Natl Acad Sci USA. 2005;102:13944-13949. 6. Linsley P S, Schelter J, Burchard J, et al. Transcripts targeted by the microRNA-16 family cooperatively regulate cell cycle progression. Mol Cell Biol. 2007;27:2240-2252. 7. Martello G, Zacchigna L, Inui M, et al. MicroRNA control of Nodal signalling. Nature. 2007;449:183-188. 8. Choong M L, Yang H H, McNiece I. MicroRNA expression profiling during human cord blood-derived CD34 cell erythropoiesis. Exp Hematol. 2007;35:551-564. 9. Georgantas R W, 3rd, Hildreth R, Morisot S, et al. CD34+ hematopoietic stem-progenitor cell microRNA expression and function: a circuit diagram of differentiation control. Proc Natl Acad Sci USA. 2007;104:2750-2755. 10. Georgantas R W, 3rd, Tanadve V, Malehorn M, et al. Microarray and serial analysis of gene expression analyses identify known and novel transcripts overexpressed in hematopoietic stem cells. Cancer Res. 2004;64:4434-4441. 11. Yu X, Alder J K, Chun J H, et al. HES1 inhibits cycling of hematopoietic progenitor cells via DNA binding. Stem Cells. 2006;24:876-888. 12. Yu X, Zhan X, D'Costa J, et al. Lentiviral vectors with two independent internal promoters transfer high-level expression of multiple transgenes to human hematopoietic stem-progenitor cells. Mol Ther. 2003;7:827-838. 13. Xie P, Chan F S, Ip N Y, Leung M F. Induction of gp130 and LIF by differentiation inducers in human myeloid leukemia K562 cells. Leuk Res. 1999;23:1113-1119. 14. Amanchy R, Kalume D E, Iwahori A, Zhong J, Pandey A. Phosphoproteome analysis of HeLa cells using stable isotope labeling with amino acids in cell culture (SILAC). J Proteome Res. 2005;4:1661-1671. 15. Ong S E, Blagoev B, Kratchmarova I, et al. Stable isotope labeling by amino acids in cell culture, SILAC, as a simple and accurate approach to expression proteomics. Mol Cell Proteomics. 2002;1:376-386. 16. Loken M R, Shah V O, Dattilio K, Civin C I. Flow Cytometric Analysis of Human Bone Marrow: I. Normal Erythroid Development. Blood. 1987;69:255-263.

### Example 5

### miRNAs Expressed by All Stem Cells

Undifferentiated human embryonic stem cells (hESCs) expressed a small set of “ES-microRNAs”, and mir-155 was expressed in day 9 and 14 EBs: Recently, the ability of hESCs 110 to differentiate into hematopoietic lineage cells has been demonstrated in several laboratories, including our own. Differentiation of hESCs, via EBs or upon co-culture with the S17 or OP9 stromal cell line, can give rise to endothelial and hematopoietic cells including hematopoietic CFCs. Our recently reported human hESC/EB-derived hematopoietic model, using the NIH-approved H1 (WA01) hESC cell line, exhibits both waves (primitive and definitive) of human hematopoiesis. Differentiation into hematopoietic cells can be evaluated using cytological staining, monoclonal antibody (Mab)/flow cytometric evaluation for lineage markers, qRT-PCR for hematopoietic transcription factors, and CFC assays. Similar results have been obtained using H9 (WA09) hESCs (unpublished).

Profiling microRNA expression in hematopoietic cells developing from hESCs: Based on our extensive experience with hESC-derived hematopoiesis, we generated and immuno-purified undifferentiated hESCs and differentiating EB cells over a time course of hESC to EB differentiation^{90,111}, and microRNAs and mRNAs were assessed in these cells by microarrays and/or qRT-PCR and BioPlex assays. hESCs were transferred from “pluripotent” growth conditions to “EB conditions” where, without PMEFs or cytokines, EBs form and spontaneous differentiation occurs. In characterizing hem-endothelial development in this model system (Zambidis et al. (2005) Blood 106:860), we established that (a) expression of hematopoiesis-associated mRNAs/proteins begins at ˜1 week of EB culture, when these cells produce mainly erythroid progeny containing embryonic hemoglobins (suggesting “embryonic” or “primitive” hematopoiesis); and (b) characteristics of “adult” (or “definitive”) hematopoiesis, such as generation of increased numbers of mono-granulocytic progenitors plus erythroid progenitors producing cells containing fetal and adult hemoglobins, are present at 9-20+ days. Therefore, the following FACS-enriched hematopoietic cell-enriched subsets were harvested at the following critical time points (Zambidis et al. (2005) Blood 106:860):
(i) Undifferentiated hESCs (CD9⁺/TRA-160⁺ at EB day 0),
(ii) Embryonic (“primitive”) erythroid-enriched cells (CD34⁺/CD31⁻/CD13⁻ at EB day 6),
(iii) Embryonic plus some adult-type (“definitive”) erythroid-enriched cells (CD34⁺/CD31⁻/CD13⁻ at EB day 9), and
(iv) Embryonic and adult type erythroid-enriched cells (CD34⁺/CD31⁻/CD13⁻ at EB day 14).

We have obtained enough cells at each of these time points to perform multiple FACS-analyses and qRT-PCR assays (Zambidis et al. (2005) Blood 106:860). These experiments were done using H1 and then H9 hESC lines. Data from hESCs were entered into our TID, and the microRNAs expressed in both hESC lines were prioritized for comparisons to the subsets obtained from primary adult human and mouse hematopoietic tissues.

Informatic analyses using the TID: Once microRNA and mRNA expression data was obtained, we normalized and analyze the data, using 2 software tools developed by Dr. Georgantas (Georgantas et al. (2007) PNAS 104:2750 and Georgantas et al. (2004) Cancer Res. 64:4434), which can (a) identify changes in microRNA and mRNA expression between chosen sources of hematopoietic cells and stages of erythroid differentiation, and (b) predict, at each of these stages, the effect of individual microRNAs on erythropoiesis by predicting their effects on specific mRNA targets expressed by the same cells.

We found only 9 microRNAs that were expressed in both H1 and H9 hESCs during growth under undifferentiated (“pluripotent”) conditions. 8 of the 9 “ES-microRNAs” are also HE-microRNAs (1 of these 9 (mir-212) was just below the expression level threshold in CD34+ cells; Table 1). We hypothesize that these ES/HE-microRNAs may regulate general sternness properties shared by other types of stem cells. Note also that the ES-microRNAs do not include the mir-155 HE-microRNA or certain other HE-microRNAs predicted to control hematopoiesis: mir-16, -146, -181, or -128; this leads us to suspect that these 5 microRNAs may be more specific for the regulation of hematopoietic differentiation than of general sternness.

Neural and mesenchymal ste r n cells were also investigated. Human neural stem cells (NSC) and mesenchymal stem cells (MSC) were isolated as described in Science 284(5411):143-147 and Stem Cells 24(12)2851-2857. The results indicated that the same miRNAs expressed in human embryonic stem cells were also found to be expressed and forming a similar signature in neural and mesenchyman stem cells (see Table 6).

TABLE 6
MicroRNAs Expressed by All Stem Cells

| | CD34⁺ | | | | |
|---|---|---|---|---|---|
| | HSPCs | | | | |
| | PBSC | | | | |
| microRNA | RFU² | BM RFU | hESCs | NSC | MSC |
| hsa-let-7b | 163,441 | 202,826 | 87,674 | 134,000 | 175,374 |
| hsa-mir-17 | 169,601 | 839,361 | 87,521 | 2,612,500 | 585,167 |
| hsa-mir-92-1 | 4,115,379 | 5,793,765 | 478,045 | 4,290,750 | 3,146,564 |
| hsa-mir-92- | 3,477,367 | 4,760,875 | 638,159 | 4,810,875 | 3,475,815 |
| 2-p | | | | | |
| hsa-mir-130a | 186,958 | 420,271 | 95,444 | 488,850 | 388,921 |
| hsa-mir-193 | 104,803 | 481,103 | 131,861 | 116,025 | 256,445 |
| hsa-mir-197 | 182,695 | 231,027 | 84,921 | 167,700 | 145,645 |
| hsa-mir-212 | 81,476 | 80,414 | 95,013 | 172,350 | 154,554 |
| (SEQ ID | | | | | |
| NO: 34) | | | | | |
| hsa-mir-222 | 354,343 | 200,347 | 94,847 | 703,500 | 612,315 |

### Example 6

### miRNA-155 is Expressed in Early Hematopoietic Stem Cells

Using qRT-PCR, we assessed levels of mir-155 at 2 time points of EB differentiation of H1 hESCs90. We had previously shown that, in day 9 EBs, hematopoietic cells are mainly embryonic (“primitive”) whereas adult (“definitive”) hematopoietic cells are present by day 14. mir-155 was not detected in undifferentiated hESCs but was expressed at days 9 and 14 of EB differentiation, further circumstantially implicating mir-155 as a hematopoiesis-associated microRNA. Based on TID-predicted target mRNAs, mir-155 may control embryonic hematopoiesis. For example, mir-155 has been recently reported to down-regulate angiotensin receptor II (AGTRII); AGTRII is a new marker for, and may be mechanistically involved in, early embryonic hematopoiesis. Therefore, we have transduced H1 hESCs with our mir-155 lentivector. To date, we have observed no difference in the growth rate of mir-155-transduced vs control-transduced hESCs, under “pluripotent” conditions.

Thus, mir-155 allows hESCs to develop into early embryonic HSPCs, but inhibits further differentiation. These differentiation-arrested cells remain able to expand.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method for modulating the differentiation of a stem-progenitor cell (SPC), comprising contacting the stem-progenitor cell with one or more nucleic acid molecules, wherein each of the nucleic acid molecules comprises a nucleotide sequence that is at least about 90% identical to a sequence selected from the group consisting of SEQ ID NOs: 1-34 and the complements thereof.

2. The method of claim 1, wherein the differentiation of the stem-progenitor cell is inhibited, and and wherein each of the nucleic acid molecules comprises a nucleotide sequence that is at least about 90% identical to a sequence selected from the group consisting of SEQ ID NOs: 1-34.

3. The method of claim 1, wherein the differentiation of the stem-progenitor cell is stimulated, and and wherein each of the nucleic acid molecules comprises a nucleotide sequence that is at least about 90% identical to a sequence selected from the group consisting of the complements of SEQ ID NOs: 1-34.

4. The method of claim 1, wherein the stem-progenitor cell is a hematopoietic stem-progenitor cell and the nucleotide sequences are selected from the group consisting of the sequences of mir-128a, mir-181a, mir-146, mir-155, mir-24a, mir-17, mir-16, mir-103, mir-107, mir-223, mir-221 and mir-222 and the complements thereof.

5. The method of claim 4, wherein the hematopoietic stem-progenitor cell is CD34+.

6. The method of claim 4, wherein the differentiation of the hematopoietic stem-progenitor cell beyond the stage of multipotent progenitor cell (MPP) and/or before the stage of common lymphoic progenitor (CLP) or common myeloid progenitor (CMP) is inhibited, and the nucleotide sequences are selected from the group consisting of the sequences of mir-128a, mir-181a, mir-146, mir-155, mir-24a, and mir-17.

7-8. (canceled)

9. The method of claim 4, wherein the differentiation of the hematopoietic stem-progenitor cell beyond the stage of CMP and/or before the stage of B/Mac bi-potential is inhibited, and the nucleotide sequences are selected from the group consisting of the sequences of mir-103 and mir-107.

10. The method of claim 4, wherein the differentiation of the hematopoietic stem-progenitor cell beyond the stage of CMP and/or before the stage of megakaryocyte-erythrocyte progenitor (MEP) is inhibited, and the polynucleotide sequence is that of mir-16.

11. The method of claim 4, wherein the differentiation of the hematopoietic stem-progenitor cell beyond the stage of granulocyte-macrophage progenitor cell (GMP) and/or before the stage of granulocytes is inhibited, and the polynucleotide sequence is that of mir-223.

12. The method of claim 4, wherein the differentiation of the hematopoietic stem-progenitor cell beyond the stage of MEP and/or before the stage of erythrocyte is inhibited, and the nucleotide sequences are selected from the group consisting of the sequences of mir-221 and mir-222.

13. The method of claim 4, wherein the differentiation of the hematopoietic stem-progenitor cell towards the stage of CLP and CMP is stimulated, and the nucleotide sequences are selected from the group consisting of mir-128a, mir-181a, mir-146, mir-155, mir-24a, and mir-17.

14-15. (canceled)

16. The method of claim 4, wherein the differentiation of the hematopoietic stem-progenitor cell towards the stage of B/Mac bi-potential and GMP is stimulated, and the nucleotide sequences are selected from the group consisting of the sequences of mir-103 and mir-107.

17. The method of claim 4, wherein the differentiation of the hematopoietic stem-progenitor cell towards the stage of CMP and/or before the stage of MEP is stimulated, and the polynucleotide sequence is that of mir-16.

18. The method of claim 4, wherein the differentiation of the hematopoietic stem-progenitor cell towards the stage of granulocyte is stimulated, and the polynucleotide sequence is that of mir-223.

19. The method of claim 4, wherein the differentiation of the hematopoietic stem-progenitor cell towards the stage of erythrocyte is stimulated, and the polynucleotide sequences are selected from the group consisting of the sequences of mir-221 and mir-222.

20. The method of claim 1, wherein the stem-progenitor cell is a human embryonic stem cell (ESC), a neural stem cell or a mesenchymal stem cell, and the nucleotide sequences are selected from the group consisting of the sequences of let-7b, mir-17, mir-92-1, mir-92-2-p, mir-130a, mir-193, mir-197, mir-212 and mir-222 and the complements thereof.

21-22. (canceled)

23. The method of claim 1, wherein the contacting is performed in vitro.

24. (canceled)

25. The method of claim 4, wherein the cell is a peripheral blood stem-progenitor cell or a bone marrow cell.

26-40. (canceled)

41. An isolated stem-progenitor cell comprising a heterologous nucleic acid comprises a nucleotide sequence that is at least about 90% identical to a sequence selected from the group consisting of the SEQ ID NOs: 1-34 and the complements thereof.

42. (canceled)

43. The isolated stem-progenitor cell of claim 41, which is a hematopoietic cell.

44-54. (canceled)

55. A method of screening a candidate substance for an effect on hematopoietic cell differentiation, comprising providing a hematopoietic cell that expresses one or more nucleic acids of SEQ ID NOS: 1-34; contacting said hematopoietic cell with said candidate substance; and assessing the effect of said candidate substance on the expression or stability of one or more nucleic acids of SEQ ID NOS: 1-34.

56. (canceled)


SEQUENCE LISTING (120 sequences)

SEQ ID NO: 1 - RNA, 22 nt, Homo sapiens
ugagguaguagguugugugguu

SEQ ID NO: 2 - RNA, 23 nt, Homo sapiens
uacccuguagauccgaauuugug

SEQ ID NO: 3 - RNA, 22 nt, Homo sapiens
uagcagcacguaaauauuggcg

SEQ ID NO: 4 - RNA, 23 nt, Homo sapiens
caaagugcuuacagugcagguag

SEQ ID NO: 5 - RNA, 23 nt, Homo sapiens
uaaagugcuuauagugcagguag

SEQ ID NO: 6 - RNA, 21 nt, Homo sapiens
aucacauugccagggauuucc

SEQ ID NO: 7 - RNA, 21 nt, Homo sapiens
aucacauugccagggauuacc

SEQ ID NO: 8 - RNA, 22 nt, Homo sapiens
uggcucaguucagcaggaacag

SEQ ID NO: 9 - RNA, 22 nt, Homo sapiens
cauugcacuugucucggucuga

SEQ ID NO: 10 - RNA, 22 nt, Homo sapiens
uucaaguaauccaggauaggcu

SEQ ID NO: 11 - RNA, 21 nt, Homo sapiens
uucaaguaauucaggauaggu

SEQ ID NO: 12 - RNA, 22 nt, Homo sapiens
uguaaacauccuacacucagcu

SEQ ID NO: 13 - RNA, 22 nt, Homo sapiens
uguaaacauccccgacuggaag

SEQ ID NO: 14 - RNA, 22 nt, Homo sapiens
uauugcacuugucccggccugu

SEQ ID NO: 15 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 16 - RNA, 23 nt, Homo sapiens
caaagugcuguucgugcagguag

SEQ ID NO: 17 - RNA, 23 nt, Homo sapiens
agcagcauuguacagggcuauga

SEQ ID NO: 18 - RNA, 23 nt, Homo sapiens
agcagcauuguacagggcuauga

SEQ ID NO: 19 - RNA, 23 nt, Homo sapiens
aaaagugcuuacagugcagguag

SEQ ID NO: 20 - RNA, 23 nt, Homo sapiens
agcagcauuguacagggcuauca

SEQ ID NO: 21 - RNA, 21 nt, Homo sapiens
ucacagugaaccggucucuuu

SEQ ID NO: 22 - RNA, 22 nt, Homo sapiens
cagugcaauguuaaaagggcau

SEQ ID NO: 23 - RNA, 22 nt, Homo sapiens
ugagaacugaauuccauggguu

SEQ ID NO: 24 - RNA, 23 nt, Homo sapiens
uuaaugcuaaucgugauaggggu

SEQ ID NO: 25 - RNA, 23 nt, Homo sapiens
aacauucaacgcugucggugagu

SEQ ID NO: 26 - RNA, 23 nt, Homo sapiens
caacggaaucccaaaagcagcug

SEQ ID NO: 27 - RNA, 22 nt, Homo sapiens
ugggucuuugcgggcgagauga

SEQ ID NO: 28 - RNA, 22 nt, Homo sapiens
uucaccaccuucuccacccagc

SEQ ID NO: 29 - RNA, 23 nt, Homo sapiens
aacauucaacgcugucggugagu

SEQ ID NO: 30 - RNA, 23 nt, Homo sapiens
agcuacauugucugcuggguuuc

SEQ ID NO: 31 - RNA, 21 nt, Homo sapiens
agcuacaucuggcuacugggu

SEQ ID NO: 32 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 33 - RNA, 22 nt, Homo sapiens
ugucaguuugucaaauacccca

SEQ ID NO: 34 - RNA, 21 nt, Homo sapiens
uaacagucuccagucacggcc

SEQ ID NO: 35 - RNA, 22 nt, Homo sapiens
acugcagugaaggcacuuguag

SEQ ID NO: 36 - RNA, 22 nt, Homo sapiens
uauugcacucgucccggccucc

SEQ ID NO: 37 - RNA, 22 nt, Homo sapiens
ugagaacugaauuccauaggcu

SEQ ID NO: 38 - RNA, 22 nt, Homo sapiens
aacuggcccucaaagucccgcu

SEQ ID NO: 39 - RNA, 83 nt, Homo sapiens
cggggugagguaguagguugugugguuucagggcagugauguugccccucggaagauaacuauacaaccuacugccuucccug

SEQ ID NO: 40 - RNA, 110 nt, Homo sapiens
gaucugucugucuucuguauauacccuguagauccgaauuuguguaaggaauuuuguggucacaaauucguaucuaggggaauauguaguugacauaaacacuccgcucu

SEQ ID NO: 41 - RNA, 89 nt, Homo sapiens
gucagcagugccuuagcagcacguaaauauuggcguuaagauucuaaaauuaucuccaguauuaacugugcugcugaaguaagguugac

SEQ ID NO: 42 - RNA, 84 nt, Homo sapiens
gucagaauaaugucaaagugcuuacagugcagguagugauaugugcaucuacugcagugaaggcacuuguagcauuauggugac

SEQ ID NO: 43 - RNA, 84 nt, Homo sapiens; identical to SEQ ID NO: 42

SEQ ID NO: 44 - RNA, 71 nt, Homo sapiens
guagcacuaaagugcuuauagugcagguaguguuuaguuaucuacugcauuaugagcacuuaaaguacugc

SEQ ID NO: 45 - RNA, 73 nt, Homo sapiens
ggccggcugggguuccuggggaugggauuugcuuccugucacaaaucacauugccagggauuuccaaccgacc

SEQ ID NO: 46 - RNA, 97 nt, Homo sapiens
cucaggugcucuggcugcuuggguuccuggcaugcugauuugugacuuaagauuaaaaucacauugccagggauuaccacgcaaccacgaccuuggc

SEQ ID NO: 47 - RNA, 68 nt, Homo sapiens
cuccggugccuacugagcugauaucaguucucauuuuacacacuggcucaguucagcaggaacaggag

SEQ ID NO: 48 - RNA, 84 nt, Homo sapiens
ggccaguguugagaggcggagacuugggcaauugcuggacgcugcccugggcauugcacuugucucggucugacagugccggcc

SEQ ID NO: 49 - RNA, 77 nt, Homo sapiens
guggccucguucaaguaauccaggauaggcugugcaggucccaaugggccuauucuugguuacuugcacggggacgc

SEQ ID NO: 50 - RNA, 84 nt, Homo sapiens
ggcuguggcuggauucaaguaauccaggauaggcuguuuccaucugugaggccuauucuugauuacuuguuucuggaggcagcu

SEQ ID NO: 51 - RNA, 77 nt, Homo sapiens
ccgggacccaguucaaguaauucaggauagguugugugcuguccagccuguucuccauuacuuggcucggggaccgg

SEQ ID NO: 52 - RNA, 77 nt, Homo sapiens; identical to SEQ ID NO: 51

SEQ ID NO: 53 - RNA, 70 nt, Homo sapiens
guuguuguaaacauccccgacuggaagcuguaagacacagcuaagcuuucagucagauguuugcugcuac

SEQ ID NO: 54 - RNA, 78 nt, Homo sapiens
cuuucuacacagguugggaucgguugcaaugcuguguuucuguaugguauugcacuugucccggccuguugaguuugg

SEQ ID NO: 55 - RNA, 75 nt, Homo sapiens
ucaucccuggguggggauuuguugcauuacuuguguucuauauaaaguauugcacuugucccggccuguggaaga

SEQ ID NO: 56 - RNA, 80 nt, Homo sapiens
cugggggcuccaaagugcuguucgugcagguagugugauuacccaaccuacugcugagcuagcacuucccgagcccccgg

SEQ ID NO: 57 - RNA, 78 nt, Homo sapiens
uacugcccucggcuucuuuacagugcugccuuguugcauauggaucaagcagcauuguacagggcuaugaaggcauug

SEQ ID NO: 58 - RNA, 78 nt, Homo sapiens
uugugcuuucagcuucuuuacagugcugccuuguagcauucaggucaagcagcauuguacagggcuaugaaagaacca

SEQ ID NO: 59 - RNA, 81 nt, Homo sapiens
ccuuggccauguaaaagugcuuacagugcagguagcuuuuugagaucuacugcaauguaagcacuucuuacauuaccaugg

SEQ ID NO: 60 - RNA, 81 nt, Homo sapiens
cucucugcuuucagcuucuuuacaguguugccuuguggcauggaguucaagcagcauuguacagggcuaucaaagcacaga

SEQ ID NO: 61 - RNA, 82 nt, Homo sapiens
ugagcuguuggauucggggccguagcacugucugagagguuuacauuucucacagugaaccggucucuuuuucagcugcuuc

SEQ ID NO: 62 - RNA, 89 nt, Homo sapiens
ugcugcuggccagagcucuuuucacauugugcuacugucugcaccugucacuagcagugcaauguuaaaagggcauuggccguguagug

SEQ ID NO: 63 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 64 - RNA, 73 nt, Homo sapiens
ccuggcacugagaacugaauuccauaggcugugagcucuagcaaugcccuguggacucaguucuggugcccgg

SEQ ID NO: 65 - RNA, 65 nt, Homo sapiens
cuguuaaugcuaaucgugauagggguuuuugccuccaacugacuccuacauauuagcauuaacag

SEQ ID NO: 66 - RNA, 110 nt, Homo sapiens
ugaguuuugagguugcuucagugaacauucaacgcugucggugaguuuggaauuaaaaucaaaaccaucgaccguugauuguacccuauggcuaaccaucaucuacucca

SEQ ID NO: 67 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 68 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 69 - RNA, 88 nt, Homo sapiens
cgaggaugggagcugagggcugggucuuugcgggcgagaugagggugucggaucaacuggccuacaaagucccaguucucggcccccg

SEQ ID NO: 70 - RNA, 83 nt, Homo sapiens
guggucucagaaucgggguuuugagggcgagaugaguuuauguuuuauccaacuggcccucaaagucccgcuuuuggggucau

SEQ ID NO: 71 - RNA, 75 nt, Homo sapiens
ggcugugccggguagagagggcagugggagguaagagcucuucacccuucaccaccuucuccacccagcauggcc

SEQ ID NO: 72 - RNA, 110 nt, Homo sapiens
cggggcaccccgcccggacagcgcgccggcaccuuggcucuagacugcuuacugcccgggccgcccucaguaacagucuccagucacggccaccgacgccuggccccgcc

SEQ ID NO: 73 - RNA, 110 nt, Homo sapiens; identical to SEQ ID NO: 66

SEQ ID NO: 74 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 75 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 76 - DNA, 42 bp, Artificial Sequence
  Description of Artificial Sequence Synthetic primer
gcattggaattcagtcaacatgcctgccccaaacaaatatgc

SEQ ID NO: 77 - DNA, 42 bp, Artificial Sequence
  Description of Artificial Sequence Synthetic primer
ccgatacctcgaggttttacatgtaattccatagacaggggt

SEQ ID NO: 78 - DNA, 46 bp, Artificial Sequence
  Description of Artificial Sequence Synthetic primer
gcattgatcgatacagacccattgaattaataaggactggtgattc

SEQ ID NO: 79 - DNA, 47 bp, Artificial Sequence
  Description of Artificial Sequence Synthetic primer
cgatacctcgaggtcagtttggtacatgatacagattggttttgcag

SEQ ID NO: 80 - DNA, 42 bp, Artificial Sequence
  Description of Artificial Sequence Synthetic primer
gcattggaattccgcggcccccgcgcgcgtccccctgccggc

SEQ ID NO: 81 - DNA, 42 bp, Artificial Sequence
  Description of Artificial Sequence Synthetic primer
cgatacctcgagaaccattctcaaaaaaaagtttattaaaag

SEQ ID NO: 82 - DNA, 42 bp, Artificial Sequence
  Description of Artificial Sequence Synthetic primer
gcattggaattccacagatgtaaaagacttttttttatacga

SEQ ID NO: 83 - DNA, 42 bp, Artificial Sequence
  Description of Artificial Sequence Synthetic primer
cgatacctcgagaagtttaacatgtacttttattaacaactt

SEQ ID NO: 84 - DNA, 42 bp, Artificial Sequence
  Description of Artificial Sequence Synthetic primer
gcattggaattcggaacagatggacgtcgaggaccgggcact

SEQ ID NO: 85 - DNA, 42 bp, Artificial Sequence
  Description of Artificial Sequence Synthetic primer
cgatacctcgagacaaaaagtattttattattcttaacagta

SEQ ID NO: 86 - DNA, 42 bp, Artificial Sequence
  Description of Artificial Sequence Synthetic primer
gcattggaattcagccctgctcgatgctcacagggcccccag

SEQ ID NO: 87 - DNA, 42 bp, Artificial Sequence
  Description of Artificial Sequence Synthetic primer
cgatacctcgagaatgcatcaaacaactgtggccagtgaaag

SEQ ID NO: 88 - DNA, 42 bp, Artificial Sequence
  Description of Artificial Sequence Synthetic primer
gcattggaattcctccggactctaaacaggaacttgaatact

SEQ ID NO: 89 - DNA, 42 bp, Artificial Sequence
  Description of Artificial Sequence Synthetic primer
cgatacctcgagctcaaataaacttccccaaaggaagatatt

SEQ ID NO: 90 - DNA, 42 bp, Artificial Sequence
  Description of Artificial Sequence Synthetic primer
gcattggaattcgtatctgagcgtttaaagtactgagcagta

SEQ ID NO: 91 - DNA, 42 bp, Artificial Sequence
  Description of Artificial Sequence Synthetic primer
cgatacctcgaggttatagttacttcaagtaacacagcttgc

SEQ ID NO: 92 - DNA, 42 bp, Artificial Sequence
  Description of Artificial Sequence Synthetic primer
gcattgatcgattgccatttgggcttatttagaaaaaagggt

SEQ ID NO: 93 - DNA, 42 bp, Artificial Sequence
  Description of Artificial Sequence Synthetic primer
cgatacctcgagagaaaagaatcaatatattttatttggcaa

SEQ ID NO: 94 - DNA, 42 bp, Artificial Sequence
  Description of Artificial Sequence Synthetic primer
gcattggaattctgaatctccaggcgacgcggttttttcact

SEQ ID NO: 95 - DNA, 42 bp, Artificial Sequence
  Description of Artificial Sequence Synthetic primer
cgatacctcgaggcttcattccacagcttttattctataaga

SEQ ID NO: 96 - DNA, 42 bp, Artificial Sequence
  Description of Artificial Sequence Synthetic primer
gcattggaattcgcccgcccagaggagcccagcggcccaaga

SEQ ID NO: 97 - DNA, 42 bp, Artificial Sequence
  Description of Artificial Sequence Synthetic primer
cgatacctcgagtggaagtcaaataaatgtttcccccactcc

SEQ ID NO: 98 - DNA, 36 bp, Artificial Sequence
  Description of Artificial Sequence Synthetic primer
gcattggaattcgggatggagaaagggcagaggaag

SEQ ID NO: 99 - DNA, 46 bp, Artificial Sequence
  Description of Artificial Sequence Synthetic primer
cgatacctcgagtccatcctttagatacacacagaatgtaaaaggg

SEQ ID NO: 100 - DNA, 30 bp, Artificial Sequence
  Description of Artificial Sequence Synthetic primer
gcatgcgaattcaaccgggccgggcacagc

SEQ ID NO: 101 - DNA, 38 bp, Artificial Sequence
  Description of Artificial Sequence Synthetic primer
cgatgcctcgagtcttataaaaacgaaccaggtagccc

SEQ ID NO: 102 - DNA, 42 bp, Artificial Sequence
  Description of Artificial Sequence Synthetic primer
gcattggaattcgcccgagtccccgggcatctttggggaggt

SEQ ID NO: 103 - DNA, 42 bp, Artificial Sequence
  Description of Artificial Sequence Synthetic primer
cgatacctcgagcagtctgtcatttttattaagcctgcagag

SEQ ID NO: 104 - DNA, 42 bp, Artificial Sequence
  Description of Artificial Sequence Synthetic primer
gcattggaattcccttcatctagttaaccaatcgcaaagcaa

SEQ ID NO: 105 - DNA, 42 bp, Artificial Sequence
  Description of Artificial Sequence Synthetic primer
cgatacctcgaggacaaaaataatcttgtttttattttagat

SEQ ID NO: 106 - DNA, 41 bp, Artificial Sequence
  Description of Artificial Sequence Synthetic primer
gcattggaattcccagaaaagcattatggttttcagaacac

SEQ ID NO: 107 - DNA, 38 bp, Artificial Sequence
  Description of Artificial Sequence Synthetic primer
cgatacctcgagtgctacaaggcagtaagtacaccgtc

SEQ ID NO: 108 - DNA, 42 bp, Artificial Sequence
  Description of Artificial Sequence Synthetic primer
gcattggaattcggcgccaggcctggcccggctgggccccgc

SEQ ID NO: 109 - DNA, 46 bp, Artificial Sequence
  Description of Artificial Sequence Synthetic primer
gcattgatcgatatggtgtatgtacttgtcaaactgtttatttgcc

SEQ ID NO: 110 - DNA, 42 bp, Artificial Sequence
  Description of Artificial Sequence Synthetic primer
gcattggaattcccgccctggtggactcctcccgctggaggc

SEQ ID NO: 111 - DNA, 42 bp, Artificial Sequence
  Description of Artificial Sequence Synthetic primer
gcattgatcgatactatttgctttcagagcacaaaacaggtt

SEQ ID NO: 112 - DNA, 42 bp, Artificial Sequence
  Description of Artificial Sequence Synthetic primer
gcattggaattcatgtctcacatagagtatctccgaatgcag

SEQ ID NO: 113 - DNA, 42 bp, Artificial Sequence
  Description of Artificial Sequence Synthetic primer
cgatacctcgagcagtagaagggagacaactgtttatgtcca

SEQ ID NO: 114 - DNA, 42 bp, Artificial Sequence
  Description of Artificial Sequence Synthetic primer
gcattggaattcccgaggccgcccctcccacgccccccagcg

SEQ ID NO: 115 - DNA, 42 bp, Artificial Sequence
  Description of Artificial Sequence Synthetic primer
cgatacctcgagacagctgctgattgtttattgcacaagaaa

SEQ ID NO: 116 - DNA, 60 bp, Artificial Sequence
  Description of Artificial Sequence Synthetic primer
ttcctgttcttaagacgtgaacagtattaattaaagctgtaaccaaagcccaaagtggta

SEQ ID NO: 117 - DNA, 60 bp, Artificial Sequence
  Description of Artificial Sequence Synthetic primer
gactgtagaaaagggaactgacagtattaattaaagctgtgtaaagctagaaatgatccc

SEQ ID NO: 118 - DNA, 60 bp, Artificial Sequence
  Description of Artificial Sequence Synthetic primer
cgagttttgtttttcaaaatacagtattaattaaagctgtatttaagtggcatcttctct

SEQ ID NO: 119 - DNA, 42 bp, Artificial Sequence
  Description of Artificial Sequence Synthetic primer
cgatcggaattcaaatggactattttcctttcagatttacta

SEQ ID NO: 120 - DNA, 42 bp, Artificial Sequence
  Description of Artificial Sequence Synthetic primer
gctatggctagctaaataaccttactttcaaaacttagttta